(19) United States
(12) Patent Application Publication
(10) Pub. No.: US 2024/0271149 A1
(43) Pub. Date: Aug. 15, 2024

(54) PLANTS PRODUCING MODIFIED LEVELS OF MEDIUM CHAIN FATTY ACIDS

(71) Applicant: Commonwealth Scientific and Industrial Research Organisation, Acton (AU)

(72) Inventors: Kyle B. Reynolds, Acton Australian Capital Territory (AU); Thomas Vanhercke, Kaleen Australian Capital Territory (AU); Anna El Tahchy, Moncrieff, Australian Capital Territory (AU); Qing Liu, Giralang Australian Capital Territory (AU); Surinder S. Singh, Downer Australian Capital Territory (AU); James R. Petrie, Goulburn New South Wales (AU)

(21) Appl. No.: 18/413,699

(22) Filed: Jan. 16, 2024

Related U.S. Application Data
(63) Continuation of application No. 16/355,215, filed on Mar. 15, 2019, now Pat. No. 11,913,006.

(30) Foreign Application Priority Data
Mar. 16, 2018 (AU) 2018201932
Mar. 16, 2018 (CA) 2998211

(51) Int. Cl. C12N 15/82; C10G 3/00; C10L 1/02; C12N 9/10; C12N 9/16; C12N 15/52

(52) U.S. Cl. CPC C12N 15/8247; C10G 3/00; C10L 1/02; C10L 1/026; C12N 9/1029; C12N 9/16; C12N 15/52; C10L 2200/0476; C12Y 203/01158; C12Y 301/02014

(57) ABSTRACT

The present invention relates to methods of producing industrial products from plant lipids, particularly from vegetative parts of plants. In particular, the present invention provides oil products such as biofuel, and processes for producing these products, as well as plants having an increased level medium chain fatty acids such as lauric acid and myristic acid. In one particular embodiment, the present invention relates to combinations of modifications in a fatty acid thioesterase and one or more acyltransferases. In an embodiment, the present invention relates to a process for extracting lipids. In another embodiment, the lipid is converted to one or more hydrocarbon products in harvested plant vegetative parts to produce alkyl esters of the fatty acids which are suitable for use as a renewable biofuel.

This present application is a continuation of U.S. Ser. No. 16/355,215, filed Mar. 15, 2019, claiming priority from Australian Patent Application No 2018201932 filed on 16 Mar. 2018 and Canadian Patent Application No 2,998,211 filed on 16 Mar. 2018, the contents of each of which are incorporated herein by reference into this application.

### REFERENCE TO A SEQUENCE LISTING

This application incorporates-by-reference nucleotide and/or amino acid sequences which are present in the file named “240405_90904-A_SequenceListing_DH.xml”, which is 275,736 bytes in size, and which was created Apr. 5, 2024 in the IBM-PC machine format, having an operating system compatibility with MS-Windows, which is contained in the xml file filed Apr. 5, 2024 as part of this application.

### FIELD OF THE INVENTION

The present invention relates to methods of producing industrial products from plant lipids, particularly from vegetative parts of plants. In particular, the present invention provides oil products such as biofuel, and processes for producing these products, as well as plants having an increased level medium chain fatty acids such as lauric acid and myristic acid. In one particular embodiment, the present invention relates to combinations of modifications in a fatty acid thioesterase and one or more acyltransferases. In an embodiment, the present invention relates to a process for extracting lipids. In another embodiment, the lipid is converted to one or more hydrocarbon products in harvested plant vegetative parts to produce alkyl esters of the fatty acids which are suitable for use as a renewable biofuel.

### BACKGROUND OF THE INVENTION

Over recent years the global production of vegetable oils has experienced constant growth, with over 179 million metric tons (MMT) being produced in 2015 (OECD/FAO, 2015), with the four major oil production crops being oil palm, soybean, canola and sunflower. An important component of global oil consumption is medium-chain fatty acids (MCFA), here defined as fatty acids in the range of 6-14 carbons in length. As well as their application within the food industry MCFAs are an ideal source for biodiesel and also for a wide range of oleochemical feedstocks including pharmaceuticals, personal care products, lubricants and detergents (Arkcoll, 1988; Basiron and Weng, 2004). Currently, the predominant crop sources of MCFA-enriched oils are coconut palm and oil palm (both palm oil and palm kernel oil) (Arkcoll, 1988). The production of these crops is limited to tropical and subtropical climates. The development of new crops that can produce MCFA-enriched oils in temperate climates has been proposed (Dehesh, 2001; Eccleston et al., 1996; Reynolds et al., 2015; Tjellstrom et al., 2013; Voelker et al., 1992; Wiberg et al., 2000) as a way to meet the growing global demand for MCFA in oleochemical production, pharmaceutical applications, and personal care products.

Many studies have investigated the modification of seed oils to contain increased MCFA content, predominantly focused on the engineering of lauric acid (C12:0) (Eccleston and Ohlrogge, 1998; Knutzon et al., 1999; Voelker et al., 1992). In oilseeds the engineered pathway begins with the overexpression of a specialised thioesterase (FATB) that prematurely truncates the standard fatty acid elongation cycle within the plastid allowing export into the cytoplasm. The MCFA in the cytoplasm is available for incorporation into triacylglycerols (TAG) via the endogenous oilseed pathways which can occur via the acyl-CoA dependent reactions of the Kennedy pathway (glycerol-3-phosphate acyltransferase (GPAT), lysophosphatidic acid acyltransferase (LPAAT) and diacylglycerol acyltransferase (DGAT). Previous studies have investigated the incorporation of MCFA into seed oils following the coordinated over-expression of FATB and LPAAT, achieving up to 67% of laurate (C12:0) in seed oil (Knutzon et al., 1999). More recently, transcriptomic analyses have enabled the identification of new FATB and LPAAT genes from many Cuphea species, which have been used to both modify the fatty acid profiles and improve the incorporation of MCFA into the TAG, respectively, of transgenic Camelina sativa seeds (Kim et al., 2015a; Kim et al., 2015b).

Evidence, although, has found that endogenous TAG synthesis pathways in developing oilseeds are not ideal for incorporating MCFA into TAG (Wiberg et al., 1997; Wiberg et al., 2000), and that newly-synthesised MCFA becomes incorporated into membrane bound lipids, impeding lipid flux, agronomic performance and can even result in cell death through chlorosis (Bates et al., 2014; Voelker et al., 1996). Therefore it would seem that although MCFA can be produced in plant cells there is a poor pathway for incorporation into seed TAG. It has also been recognised that the accumulation of unusual fatty acids in PC appears to be a bottleneck for their enriched incorporation into TAG (Bates and Browse, 2011; Reynolds et al., 2015). In the example of engineering ricinoleic acid into oilseeds it has been demonstrated that the endogenous pathways need to be removed in conjunction with the ectopic expression of the specialised pathway counterpart (Adhikari et al., 2016; Bates and Browse, 2011; Burgal et al., 2008; Chen et al., 2016; van Erp et al., 2011; van Erp et al., 2015).

Recent work has demonstrated that engineering high oil levels in plant biomass is a realistic proposition (Vanhercke et al., 2014a; Vanhercke et al., 2013; Vanhercke et al., 2014b) with the accumulation of levels of TAG in Nicotiana tabacum leaves of up to 15% being attained by the coordinated transgenic expression of genes normally involved in oil production in seeds (Vanhercke et al., 2014a). Such approaches have uncovered a synergism involving an increase in the production of fatty acids in the plastid (WRINKLED1 (WRI1)), improving the assembly of fatty acids into leaf oils (DGAT) and slowing the catabolism of these oils (OLEOSIN, OLE1 (Winichayakul et al., 2013)); and sugar-dependent-1, SDP1 (Fan et al., 2014; Kelly et al., 2013a and b; Kim et al., 2014b; Vanhercke, 2014a). Although the production of TAG in biomass offers a new source of common vegetable oils, these new expression platforms could also be adapted to produce high levels of novel fatty acids, such as MCFA (Reynolds et al., 2015; Wood, 2014).

The inventors first steps in this direction involved the overexpression of thioesterases from Umbellularia californica, Cinnamomum camphora and Cocos nucifera which resulted in the production of MCFA in leaf tissues (Reynolds et al., 2015). However, these metabolic pathways also resulted in high levels of MCFA in PC resulting in severe chlorosis and cell death (Bates et al., 2014; Wiberg et al., 2000), similar to conclusions drawn from oilseed engineering. The incorporation of MCFA into the membrane lipids of vegetative tissues is therefore particularly problematic.

The inventors have improved the MCFA metabolic pathway by combining a series of gene ensembles with three different DGAT1 genes isolated from Elaeis guineensis (African oil palm). A functional GPAT9 from C. nucifera was identified that was included in the metabolic pathway for improving the incorporation of MCFA into seed oils. An improvement in MCFA utilisation was demonstrated in vegetative plant cells such as leaf cells, which resulted in more efficient sequestering of MCFA in TAG while also effectively limiting the accumulation of MCFA in membrane lipids.

### SUMMARY OF THE INVENTION

In a first aspect, the present invention provides a process for producing extracted plant lipid, comprising the steps of:
a) obtaining one or more plant parts comprising lipid, preferably vegetative plant parts, the lipid comprising a total fatty acid content which comprises fatty acids in an esterified form, the fatty acids comprising a level of total, or new, medium chain fatty acids (MCFA) that is at least 25% of the total fatty acid content on a weight basis, and
b) extracting lipid from the plant part(s),
   thereby producing the extracted plant lipid.

In an embodiment, the plant part comprises one or more exogenous polynucleotides which encode polypeptides having fatty acid thioesterase (TE) activity, and either glycerol-3-phosphate acyltransferase (GPAT) activity, preferably GPAT9 activity, or diacylglycerol acyltransferase (DGAT) activity, preferably DGAT1 activity, or both GPAT and DGAT,
wherein the exogenous polynucleotide is operably linked to a promoter which is capable of directing expression of the polynucleotide in a cell of the plant part.

In a further embodiment, the plant part further comprises one or more or all of:
i. an exogenous polynucleotide which encodes a second polypeptide having glycerol-3-phosphate acyltransferase (GPAT) activity, preferably GPAT9 activity, or diacylglycerol acyltransferase (DGAT) activity, preferably DGAT1 activity;
ii. an exogenous polynucleotide which encodes a third polypeptide having 1-acyl-glycerol-3-phosphate acyltransferase (LPAAT) activity;
iii. an exogenous polynucleotide which encodes a transcription factor polypeptide that increases the expression of one or more glycolytic and/or fatty acid biosynthetic genes in a cell of the plant part compared to a corresponding cell lacking the exogenous polynucleotide;
iv. an exogenous polynucleotide which encodes a polypeptide which increases the export of fatty acids out of plastids of a cell in the plant part when compared to a corresponding cell lacking the exogenous polynucleotide; and
v. an exogenous polynucleotide which encodes an oil body coating (OBC) polypeptide,
   wherein each exogenous polynucleotide is operably linked to a promoter which is capable of directing expression of the polynucleotide in a cell of the plant part.

In an embodiment, the OBC polypeptide is an oleosin, such as a polyoleosin or a caleosin, or a lipid droplet associated protein (LDAP).

In an embodiment, the transcription factor polypeptide is selected from the group consisting of Wrinkled 1 (WRI1), Leafy Cotyledon 1 (LEC1), LEC1-like, Leafy Cotyledon 2 (LEC2), BABY BOOM (BBM), FUS3, ABI3, ABI4, ABI5, Dof4 and Dof11, preferable WRI1, or the group consisting of MYB73, bZIP53, AGL15, MYB115, MYB118, TANMEI, WUS, GFR2al, GFR2a2 and PHR1.

In an embodiment, the polypeptide which increases the export of fatty acids out of plastids of the cell is a fatty acid thioesterase such as a FATA polypeptide or a FATB polypeptide, a fatty acid transporter such as an ABCA9 polypeptide or a long-chain acyl-CoA synthetase (LACS), preferably a FATB polypeptide.

In an embodiment, the fatty acid thioesterase is capable of hydrolysing a substrate which is an acyl carrier protein (ACP) esterified to a medium chain fatty acid and/or a C16:0, preferably wherein the MCFA is a C10, C12 and/or C14.

In an embodiment, the plant part further comprises one or more or all of:
i. a genetic modification which down-regulates endogenous production and/or activity of a polypeptide involved in the catabolism of triacylglycerols (TAG) in a cell of the plant part when compared to a corresponding cell lacking the genetic modification;
ii. a genetic modification which down-regulates endogenous production and/or activity of a polypeptide involved in importing fatty acids into plastids of a cell in the plant part when compared to a corresponding cell lacking the genetic modification; and
iii. a genetic modification which down-regulates endogenous production and/or activity of a polypeptide involved in diacylglycerol (DAG) production in the plastid when compared to a corresponding cell in the plant part lacking the genetic modification.

In an embodiment, the polypeptide involved in the catabolism of triacylglycerols (TAG) in the plant, or part thereof, is an SDP1 lipase, a Cgi58 polypeptide, an acyl-CoA oxidase such as ACX1 or ACX2, or a polypeptide involved in β-oxidation of fatty acids in the plant or part thereof such as a PXA1 peroxisomal ATP-binding cassette transporter, preferably an SDP1 lipase.

In an embodiment, the polypeptide involved in importing fatty acids into plastids of the cell is a fatty acid transporter, or subunit thereof, preferably a TGD polypeptide.

In an embodiment, the polypeptide involved in diacylglycerol (DAG) production in the plastid is a plastidial GPAT, a plastidial LPAAT or a plastidial PAP.

In another embodiment, the plant part further comprises one or both of:
i. an exogenous polynucleotide which encodes a second transcription factor polypeptide that increases the expression of one or more glycolytic and/or fatty acid biosynthetic genes in the cell; and
ii. a second genetic modification which down-regulates endogenous production and/or activity of a polypeptide involved in importing fatty acids into plastids of the cell when compared to a corresponding cell lacking the second genetic modification.

In a preferred embodiment, the presence of a) a genetic modification which down-regulates endogenous production and/or activity of a polypeptide involved in the catabolism of triacylglycerols (TAG) in a cell of the plant part when compared to a corresponding cell lacking the genetic modification, b) an exogenous polynucleotide which encodes a polypeptide which increases the export of fatty acids out of plastids of a cell in the plant part when compared to a corresponding cell lacking the exogenous polynucleotide, or c) an exogenous polynucleotide which encodes a second transcription factor polypeptide that increases the expression of one or more glycolytic and/or fatty acid biosynthetic genes in the cell, together with an exogenous polynucleotide which encodes a WRI1 polypeptide and an exogenous polynucleotide which encodes a polypeptide having DGAT1 activity, increases the total non-polar lipid content of the plant part, preferably a vegetative plant part such as a leaf or stem, relative to a corresponding plant part comprising the exogenous polynucleotides encoding the WRI1 and DGAT1 polypeptides but lacking each of the other exogenous polynucleotide and genetic modifications. Most preferably, at least the promoter that directs expression of the exogenous polynucleotide which encodes the transcription factor is a promoter other than a constitutive promoter. Alternatively for Sorghum or Zea mays, the promoter is preferably a constitutive promoter such as, for example a ubiquitin gene promoter.

In an embodiment, the addition of one or more of the exogenous polynucleotides or genetic modifications, preferably the exogenous polynucleotide encoding an OBC or a fatty acyl thioesterase or the genetic modification which down-regulates endogenous production and/or activity of a polypeptide involved in the catabolism of triacylglycerols (TAG) in the plant or part thereof, more preferably the exogenous polynucleotide which encodes a FATA thioesterase or an LDAP or which decreases expression of an endogenous TAG lipase such as a SDP1 TAG lipase in the plant or part thereof, results in a synergistic increase in the total non-polar lipid content of the plant or part thereof when added to the pair of transgenes WRI1 and DGAT, particularly before the plant flowers and even more particularly in the stems and/or roots of the plant.

In a preferred embodiment, the increase in the TAG content of a stem or root is at least 2-fold, more preferably at least 3-fold, relative to a corresponding plant part transformed with genes encoding WRI1 and DGAT1 but lacking the FATA thioesterase, LDAP and the genetic modification which down-regulates endogenous production and/or activity of a polypeptide involved in the catabolism of triacylglycerols (TAG) in the plant part. Most preferably, at least the promoter that directs expression of the exogenous polynucleotide which encodes the transcription factor is a promoter other than a constitutive promoter. Alternatively for Sorghum or Zea mays, the promoter is preferably a constitutive promoter such as, for example a ubiquitin gene promoter.

In an embodiment, each genetic modification is, independently, a mutation of an endogenous gene which partially or completely inactivates the gene, such as a point mutation, an insertion, or a deletion, or an exogenous polynucleotide encoding an RNA molecule which inhibits expression of the endogenous gene, wherein the exogenous polynucleotide is operably linked to a promoter which is capable of directing expression of the polynucleotide in the plant, or part thereof. The point mutation may be a premature stop codon, a splice-site mutation, a frame-shift mutation or an amino acid substitution mutation that reduces activity of the gene or the encoded polypeptide. The deletion may be of one or more nucleotides within a transcribed exon or promoter of the gene, or extend across or into more than one exon, or extend to deletion of the entire gene. Preferably the deletion is introduced by use of ZF, TALEN or CRISPR technologies. In an alternate embodiment, one or more or all of the genetic modifications is an exogenous polynucleotide encoding an RNA molecule which inhibits expression of the endogenous gene, wherein the exogenous polynucleotide is operably linked to a promoter which is capable of directing expression of the polynucleotide in the plant, or part thereof. Examples of exogenous polynucleotide which reduces expression of an endogenous gene are selected from the group consisting of an antisense polynucleotide, a sense polynucleotide, a microRNA, a polynucleotide which encodes a polypeptide which binds the endogenous enzyme, a double stranded RNA molecule and a processed RNA molecule derived therefrom. In an embodiment, the plant or part thereof comprises genetic modifications which are an introduced mutation in an endogenous gene and an exogenous polynucleotide encoding an RNA molecule which reduces expression of another endogenous gene. In an alternate embodiment, all of the genetic modifications that provide for the increased TTQ and or TAG levels are mutations of endogenous genes.

In an embodiment, the activity of PDCT or CPT in a cell in the plant part is increased relative to a wild-type plant part. Alternatively, the activity of PDCT or CPT is decreased, for example by mutation in the endogenous gene encoding the enzyme or by downregulation of the gene through an RNA molecule which reduces its expression.

In an embodiment, when present, the two transcription factors are WRI1 and LEC2, or WRI1 and LEC1.

In the above embodiments, the plant part preferably comprises an exogenous polynucleotide which encodes a DGAT and a genetic modification which down-regulates production of an endogenous SDP1 lipase. More preferably, the plant part does not comprise an exogenous polynucleotide encoding a PDAT, and/or is a plant part other than a Nicotiana benthamiana or part thereof, and/or the WRI1 is a WRI1 other than Arabidopsis thaliana WRI1 and/or is a plant part other than a Brassica napus or part thereof. In an embodiment, at least one of the exogenous polynucleotides in the plant part is expressed from a promoter which is not a constitutive promoter such as, for example, a promoter which is expressed preferentially in green tissues or stems of the plant or that is up-regulated after commencement of flowering or during senescence.

In an embodiment, the plant part comprises an increased level or activity of polypeptides which are:
i. a GPAT, a LPAAT, and a WRI1 polypeptide;
ii. a GPAT, a LPAAT, a DGAT and a WRI1 polypeptide;
iii. a GPAT9, a LPAAT, and a WRI1 polypeptide;
iv. a GPAT9, a LPAAT, a DGAT, and a WRI1 polypeptide;
v. a GPAT, a LPAAT, a DGAT1, and a WRI1 polypeptide;
vi. a GPAT9, a LPAAT, a DGAT1, and a WRI1 polypeptide;
vii. a GPAT, a LPAAT, a WRI1 polypeptide, and a fatty acid thioesterase, preferably a FATB polypeptide;
viii. a GPAT, a LPAAT, a DGAT, a WRI1 polypeptide, and a fatty acid thioesterase, preferably a FATB polypeptide;
ix. a GPAT9, a LPAAT, a WRI1 polypeptide, and a fatty acid thioesterase, preferably a FATB polypeptide;
x. a GPAT9, a LPAAT, a DGAT, a WRI1 polypeptide, and a fatty acid thioesterase, preferably a FATB polypeptide;
xi. a GPAT, a LPAAT, a DGAT1, a WRI1 polypeptide, and a fatty acid thioesterase, preferably a FATB polypeptide;
xii. a GPAT9, a LPAAT, a DGAT1, a WRI1 polypeptide, and a fatty acid thioesterase, preferably a FATB polypeptide;
xiii. a GPAT, a LPAAT, a WRI1 polypeptide, a fatty acid thioesterase, preferably a FATB polypeptide, and an OBC polypeptide such as an oleosin, preferably a caleosin or a LDAP;
xiv. a GPAT, a LPAAT, a DGAT, a WRI1 polypeptide, a fatty acid thioesterase, preferably a FATB polypeptide, and an OBC polypeptide such as an oleosin, preferably a caleosin or a LDAP;
xv. a GPAT9, a LPAAT, a WRI1 polypeptide, a fatty acid thioesterase, preferably a FATB polypeptide, and an OBC polypeptide such as an oleosin, preferably a caleosin or a LDAP;
xvi. a GPAT9, a LPAAT, a DGAT, a WRI1 polypeptide, a fatty acid thioesterase, preferably a FATB polypeptide, and an OBC polypeptide such as an oleosin, preferably a caleosin or a LDAP;
xvii. a GPAT, a LPAAT, a DGAT1, a WRI1 polypeptide, a fatty acid thioesterase, preferably a FATB polypeptide, and an OBC polypeptide such as an oleosin, preferably a caleosin or a LDAP;
xviii. a GPAT9, a LPAAT, a DGAT1, a WRI1 polypeptide, a fatty acid thioesterase, preferably a FATB polypeptide, and an OBC polypeptide such as an oleosin, preferably a caleosin or a LDAP;
xix. a GPAT, a LPAAT, a WRI1 polypeptide, a fatty acid thioesterase, preferably a FATB polypeptide, and a silencing RNA which reduces the expression of an endogenous gene which encodes a SDP1 lipase;
xx. a GPAT, a LPAAT, a DGAT, a WRI1 polypeptide, a fatty acid thioesterase, preferably a FATB polypeptide, and a silencing RNA which reduces the expression of an endogenous gene which encodes a SDP1 lipase;
xxi. a GPAT9, a LPAAT, a WRI1 polypeptide, a fatty acid thioesterase, preferably a FATB polypeptide, and a silencing RNA which reduces the expression of an endogenous gene which encodes a SDP1 lipase;
xxii. a GPAT9, a LPAAT, a DGAT, a WRI1 polypeptide, a fatty acid thioesterase, preferably a FATB polypeptide, and a silencing RNA which reduces the expression of an endogenous gene which encodes a SDP 1 lipase;
xxiii. a GPAT, a LPAAT, a DGAT1, a WRI1 polypeptide, a fatty acid thioesterase, preferably a FATB polypeptide, and a silencing RNA which reduces the expression of an endogenous gene which encodes a SDP1 lipase;
xxiv. a GPAT9, a LPAAT, a DGAT1, a WRI1 polypeptide, a fatty acid thioesterase, preferably a FATB polypeptide, and a silencing RNA which reduces the expression of an endogenous gene which encodes a SDP 1 lipase;
xxv. a GPAT, a LPAAT, a DGAT, a WRI1 polypeptide, a fatty acid thioesterase, preferably a FATB polypeptide, an OBC polypeptide such as an oleosin, preferably a caleosin or a LDAP, and a silencing RNA which reduces the expression of an endogenous gene which encodes a SDP1 lipase;
xxvi. a GPAT9, a LPAAT, a WRI1 polypeptide, a fatty acid thioesterase, preferably a FATB polypeptide, an OBC polypeptide such as an oleosin, preferably a caleosin or a LDAP, and a silencing RNA which reduces the expression of an endogenous gene which encodes a SDP1 lipase;
xxvii. a GPAT9, a LPAAT, a DGAT, a WRI1 polypeptide, a fatty acid thioesterase, preferably a FATB polypeptide, an OBC polypeptide such as an oleosin, preferably a caleosin or a LDAP, and a silencing RNA which reduces the expression of an endogenous gene which encodes a SDP1 lipase;
xxviii. a GPAT, a LPAAT, a DGAT1, a WRI1 polypeptide, a fatty acid thioesterase, preferably a FATB polypeptide, an OBC polypeptide such as an oleosin, preferably a caleosin, or a LDAP, and a silencing RNA which reduces the expression of an endogenous gene which encodes a SDP1 lipase; or a GPAT9, a LPAAT, a DGAT1, a WRI1 polypeptide, a fatty acid thioesterase, preferably a FATB polypeptide, an OBC polypeptide such as an oleosin, preferably a caleosin, or a LDAP, and a silencing RNA which reduces the expression of an endogenous gene which encodes a SDP1 lipase.

In an embodiment, the one or more or all of the polypeptides are encoded by one or more exogenous polynucleotides in the plant parts.

In a second aspect, the present invention provides a process for producing extracted plant lipid, comprising the steps of:
a) obtaining one or more plant parts comprising lipid, preferably vegetative plant parts, the lipid comprising a total fatty acid content which comprises fatty acids in an esterified form, the fatty acids comprising an increased level of medium chain fatty acids (MCFA) relative to a corresponding wild-type plant part, wherein the plant part comprises an increased level or activity of polypeptides which are:
i. a GPAT, a LPAAT, and a WRI1 polypeptide;
ii. a GPAT, a LPAAT, a DGAT and a WRI1 polypeptide;
iii. a GPAT9, a LPAAT, and a WRI1 polypeptide;
iv. a GPAT9, a LPAAT, a DGAT, and a WRI1 polypeptide;
v. a GPAT, a LPAAT, a DGAT1, and a WRI1 polypeptide;
vi. a GPAT9, a LPAAT, a DGAT1, and a WRI1 polypeptide;
vii. a GPAT, a LPAAT, a WRI1 polypeptide, and a fatty acid thioesterase, preferably a FATB polypeptide;
viii. a GPAT, a LPAAT, a DGAT, a WRI1 polypeptide, and a fatty acid thioesterase, preferably a FATB polypeptide;
ix. a GPAT9, a LPAAT, a WRI1 polypeptide, and a fatty acid thioesterase, preferably a FATB polypeptide;
x. a GPAT9, a LPAAT, a DGAT, a WRI1 polypeptide, and a fatty acid thioesterase, preferably a FATB polypeptide;
xi. a GPAT, a LPAAT, a DGAT1, a WRI1 polypeptide, and a fatty acid thioesterase, preferably a FATB polypeptide;
xii. a GPAT9, a LPAAT, a DGAT1, a WRI1 polypeptide, and a fatty acid thioesterase, preferably a FATB polypeptide;
xiii. a GPAT, a LPAAT, a WRI1 polypeptide, a fatty acid thioesterase, preferably a FATB polypeptide, and an OBC polypeptide such as an oleosin, preferably a caleosin or a LDAP;
xiv. a GPAT, a LPAAT, a DGAT, a WRI1 polypeptide, a fatty acid thioesterase, preferably a FATB polypeptide, and an OBC polypeptide such as an oleosin, preferably a caleosin or a LDAP;
xv. a GPAT9, a LPAAT, a WRI1 polypeptide, a fatty acid thioesterase, preferably a FATB polypeptide, and an OBC polypeptide such as an oleosin, preferably a caleosin or a LDAP; a GPAT9, a LPAAT, a DGAT, a WRI1 polypeptide, a fatty acid thioesterase, preferably a xvi. FATB polypeptide, and an OBC polypeptide such as an oleosin, preferably a caleosin or a LDAP;
xvii. a GPAT, a LPAAT, a DGAT1, a WRI1 polypeptide, a fatty acid thioesterase, preferably a FATB polypeptide, and an OBC polypeptide such as an oleosin, preferably a caleosin or a LDAP;
xviii. a GPAT9, a LPAAT, a DGAT1, a WRI1 polypeptide, a fatty acid thioesterase, preferably a FATB polypeptide, and an OBC polypeptide such as an oleosin, preferably a caleosin or a LDAP;
xix. a GPAT, a LPAAT, a WRI1 polypeptide, a fatty acid thioesterase, preferably a FATB polypeptide, and a silencing RNA which reduces the expression of an endogenous gene which encodes a SDP1 lipase;
xx. a GPAT, a LPAAT, a DGAT, a WRI1 polypeptide, a fatty acid thioesterase, preferably a FATB polypeptide, and a silencing RNA which reduces the expression of an endogenous gene which encodes a SDP1 lipase;
xxi. a GPAT9, a LPAAT, a WRI1 polypeptide, a fatty acid thioesterase, preferably a FATB polypeptide, and a silencing RNA which reduces the expression of an endogenous gene which encodes a SDP1 lipase;
xxii. a GPAT9, a LPAAT, a DGAT, a WRI1 polypeptide, a fatty acid thioesterase, preferably a FATB polypeptide, and a silencing RNA which reduces the expression of an endogenous gene which encodes a SDP1 lipase;
xxiii. a GPAT, a LPAAT, a DGAT1, a WRI1 polypeptide, a fatty acid thioesterase, preferably a FATB polypeptide, and a silencing RNA which reduces the expression of an endogenous gene which encodes a SDP 1 lipase;
xxiv. a GPAT9, a LPAAT, a DGAT1, a WRI1 polypeptide, a fatty acid thioesterase, preferably a FATB polypeptide, and a silencing RNA which reduces the expression of an endogenous gene which encodes a SDP 1 lipase;
xxv. a GPAT, a LPAAT, a DGAT, a WRI1 polypeptide, a fatty acid thioesterase, preferably a FATB polypeptide, an OBC polypeptide such as an oleosin, preferably a caleosin or a LDAP, and a silencing RNA which reduces the expression of an endogenous gene which encodes a SDP1 lipase;
xxvi. a GPAT9, a LPAAT, a WRI1 polypeptide, a fatty acid thioesterase, preferably a FATB polypeptide, an OBC polypeptide such as an oleosin, preferably a caleosin or a LDAP, and a silencing RNA which reduces the expression of an endogenous gene which encodes a SDP1 lipase;
xxvii. a GPAT9, a LPAAT, a DGAT, a WRI1 polypeptide, a fatty acid thioesterase, preferably a FATB polypeptide, an OBC polypeptide such as an oleosin, preferably a caleosin or a LDAP, and a silencing RNA which reduces the expression of an endogenous gene which encodes a SDP1 lipase;
xxviii. a GPAT, a LPAAT, a DGAT1, a WRI1 polypeptide, a fatty acid thioesterase, preferably a FATB polypeptide, an OBC polypeptide such as an oleosin, preferably a caleosin, or a LDAP, and a silencing RNA which reduces the expression of an endogenous gene which encodes a SDP1 lipase; or
xxix. a GPAT9, a LPAAT, a DGAT1, a WRI1 polypeptide, a fatty acid thioesterase, preferably a FATB polypeptide, an OBC polypeptide such as an oleosin, preferably a caleosin, or a LDAP, and a silencing RNA which reduces the expression of an endogenous gene which encodes a SDP1 lipase,
and
   b) extracting lipid from the plant part(s),
   thereby producing the extracted plant lipid.

In an embodiment, the one or more or all of the polypeptides are encoded by one or more exogenous polynucleotides in the plant parts.

In an embodiment, the level of total, or new, MCFA is increased relative to a corresponding wild-type plant part, preferably the level is at least 25% of the total fatty acid content on a weight basis.

In an embodiment of the first and second aspects, the one or more or all of the encoded GPAT, LPAAT and DGAT have a preference for utilising medium chain fatty acid substrates. GPAT, LPAAT and DGAT each use an acyl-CoA substrate, with a second substrate that is G3P, LPA or DAG, respectively.

In an embodiment of the first and second aspects, the extracted lipid has one or more or all of the following features:
i. the level of medium chain fatty acids in the total fatty acid content of the extracted lipid, and/or in the total fatty acid content of the TAG of the extracted lipid, is at least about 30%, at least about 35%, at least about 40%, at least about 50%, at least about 55%, or between about 25% and about 55%, between about 25% and about 50%, between about 30% and about 50%, between about 35% and about 50%, between about 25% and about 40%, or between about 30% and about 40%;
ii. the level of lauric acid (C12:0) in the total fatty acid content of the extracted lipid, and/or in the total fatty acid content of the TAG of the extracted lipid, is, or is increased by, at least about 15%, at least about 20%, at least about 25%, at least about 30%, at least about 35%, at least about 40%, at least about 50%, at least or about 55%, or between about 15% and about 55%, between about 20% and about 50%, between about 30% and about 50%, between about 35% and about 50%, between about 15% and about 25%, or between about 20% and about 30%;
iii. the level of myristic acid (C14:0) in the total fatty acid content of the extracted lipid, and/or in the total fatty acid content of the TAG of the extracted lipid, is, or is increased by, at least about 25%, at least about 30%, at least about 35%, at least about 40%, at least about 45%, or between about 25% and about 45%, between about 20% and about 50%, between about 30% and about 50%, between about 35% and about 50%, between about 30% and about 40%, between about 15% and about 25%, or between about 20% and about 30%;
iv. the level of palmitic acid (C16:0) in the total fatty acid content of the extracted lipid, and/or in the total fatty acid content of the TAG of the extracted lipid, is, or is increased by, between about 2% and about 18%, or between about 2% and about 16%, or between about 2% and about 15%, or between about 15% and about 50%;
v. the level of lauric acid (C12:0) in the total fatty acid content of the extracted lipid, and/or in the total fatty acid content of the TAG of the extracted lipid is, or is increased by, at least about 25%, at least about 30%, at least about 40%, at least about 45%, or at least about 50%, and the level of myristic acid (C14:0) in the total fatty acid content of the extracted lipid and/or in the total fatty acid content of the TAG of the extracted lipid is, or is increased by, at least about 1%, at least about 2%, at least about 5%, or at least about 10%, or between about 1% and about 10%, or between about 2% and 10%, or between about 2% and about 6%, or less than about 10%, or less than about 8% or less than about 5%, or less than about 2%;
vi. the level of myristic acid (C14:0) in the total fatty acid content of the extracted lipid, and/or in the total fatty acid content of the TAG of the extracted lipid is, or is increased by, at least about 20%, at least about 25%, at least about 30%, or at least about 40%, and the level of lauric acid (C12:0) in the total fatty acid content of the extracted lipid and/or in the total fatty acid content of the TAG of the extracted lipid is, or is increased by, at least about 1%, at least about 2%, at least about 5%, or at least about 10%, or between about 1% and about 10%, or between about 2% and about 10%, or between about 2% and about 6%, or less than about 10%, or less than about 8% or less than about 5%, or less than about 2%;
vii. the level of myristic acid (C14:0) in the total fatty acid content of the extracted lipid, and/or in the total fatty acid content of the TAG of the extracted lipid is, or is increased by, at least about 20%, at least about 25%, at least about 30%, and the level of palmitic acid (C16:0) in the total fatty acid content of the extracted lipid and/or in the total fatty acid content of the TAG of the extracted lipid is, or is increased by, at least about 2%, at least about 3%, at least about 4%, or at least about 5%.
viii. the ratio of lauric acid (C12:0):myristic acid (C14:0) in the fatty acid content of the extracted lipid, and/or in the total fatty acid content of the TAG of the extracted lipid, is increased, or is about 1:4, about 1:5, about 1:10, about 1:15, about 1:20, about 1:25, or about 4:1, about 5:1, about 10:1, about 15:1, about 20:1, about 30:1, about 40:1, or about 45:1;
ix. the ratio of lauric acid (C12:0):palmitic acid (C16:0) in the fatty acid content of the extracted lipid, and/or in the total fatty acid content of the TAG of the extracted lipid, is increased, or is about 1:2, about 1:3, about 1:4, about 1:5, about 1:10, about 1:15, about 10:1, about 20:1, about 30:1, about 40:1, or about 45:1;
x. the ratio of myristic acid (C14:0):palmitic acid (C16:0) in the fatty acid content of the extracted lipid, and/or in the total fatty acid content of the TAG of the extracted lipid, is increased, or is about 1:2, about 1:3, about 1:4, about 1:5, about 1:10, about 1:15, about 10:1, about 20:1, about 30:1, or about 40:1;
xi. the level of oleic acid in the total fatty acid content of the extracted lipid, and/or in the total fatty acid content of the TAG of the extracted lipid, is decreased, or is less than about 10%, less than about 8%, less than about 6%, less than about 5%, less than about 4%, less than about 3%, less than about 2%, less than about 1%;
xii. the level of linoleic acid (LA) in the total fatty acid content of the extracted lipid, and/or in the total fatty acid content of the TAG of the extracted lipid, is increased or decreased, or is less than about 20%, less than about 15%, less than about 10%, less than about 5%, less than about 4%, less than about 3%, less than about 2%, or less than about 1%;
xiii. the level of α-linolenic acid (ALA) in the total fatty acid content of the extracted lipid, or in the total fatty acid content of the TAG of the extracted lipid, is decreased or is less than about 50%, less than about 40%, less than about 30%, less than about 20%, less than about 10%, less than about 8%, less than about 6%, less than about 5%, less than about 2%, or less than about 1%;
xiv. the level of total unsaturated fatty acids in the total fatty acid content of the extracted lipid, and/or in the total fatty acid content of the TAG of the extracted lipid, is decreased, or is less than about 50%, less than about 40%, less than about 30%, less than about 20%, less than about 15%, less than about 10%, less than about 8%, less than about 6%, less than about 5%, less than about 2%, or less than about 1%;
xv. the level of total monounsaturated fatty acids in the total fatty acid content of the extracted lipid, and/or in the total fatty acid content of the TAG of the extracted lipid, is decreased, or is less than about 10%, less than about 5%, less than about 4%, less than about 3%, less than about 2%, or less than about 1%;
xvi. the level of total polyunsaturated fatty acids in the total fatty acid content of the extracted lipid, and/or in the total fatty acid content of the TAG of the extracted lipid, is less than about 50%, less than about 40%, less than about 30%, less than about 20%, less than about 15%, less than about 10%, less than about 8%, less than about 6%, less than about 5%, less than about 2%, or less than about 1%;
xvii. the triacylglycerol (TAG) content of the extracted lipid is at least about 80%, at least about 85%, at least about 90%, or least about 95%, and about 98%, or between about 95% and about 98%, by weight of the extracted lipid;
xviii. the TAG content of the extracted lipid comprises, or is increased in a level of, one or more or all of the TAG species 36:0, 38:0, 40:0 and 42:0;
xix. the extracted lipid comprises tri-laurin (tri-C12:0) and/or tri-myristin (tri-C14:0); and
xx. the phosphocholine (PC) content of the extracted lipid comprises one or both of the PC species 28:0 and 30:0,
   wherein any increase or decrease is relative to a corresponding wild-type plant part.

In an embodiment, the plant part comprises one or more of the features defined with respect to the first aspect.

In an embodiment of the first and second aspects, the plant part has one or more or all of the following features:
a) an increased soluble protein content relative to a corresponding wild-type plant part,
b) an increased nitrogen content in plant part relative to a corresponding wild-type plant part,
f) decreased carbon:nitrogen ratio relative to a corresponding wild-type plant part,
g) increased photosynthetic gene expression relative to a corresponding wild-type plant part,
h) increased photosynthetic capacity relative to a corresponding wild-type plant part,
i) decreased total dietary fibre (TDF) content relative to a corresponding wild-type plant part,
j) increased carbon content relative to a corresponding wild-type plant part, and
k) increased energy content relative to a corresponding wild-type plant part,
   wherein each exogenous polynucleotide is operably linked to a promoter which is capable of directing expression of the polynucleotide in the cell.

In an embodiment, the plant part, preferably a Sorghum sp. or Zea mays plant part, further comprises:
1) an increased TTQ relative to a corresponding wild-type plant part, wherein each exogenous polynucleotide is operably linked to a promoter which is capable of directing expression of the polynucleotide in the plant, or part thereof.

In an embodiment, the plant part is derived from an ancestor plant, for example, as described herein.

In an embodiment, the plant part has one or more or all of:
i) the plant part has an increased soluble protein relative to the corresponding wild-type plant part of at least about 10%, at least about 25%, at least about 50%, at least about 75%, at least about 100%, between about 10% and about 200%, between about 50% and about 150%, or between about 50% and about 125%,
ii) the plant part has an increased nitrogen content relative to the corresponding wild-type plant part of at least about 10%, at least about 25%, at least about 50%, at least about 75%, at least about 100%, between about 10% and about 200%, between about 50% and about 150% or between about 50% and about 125%,
iii) the plant part is a leaf which has an increased soluble protein content relative to a corresponding wild-type leaf of at least about 10%, at least about 25%, at least about 50%, at least about 75%, at least about 100%, between about 10% and about 200%, between about 50% and about 150%, or between about 50% and about 125%,
iv) the plant part is a leaf which has an increased nitrogen content relative to a corresponding wild-type leaf of at least about 10%, at least about 25%, at least about 50%, at least about 75%, at least about 100%, between about 10% and about 200%, between about 50% and about 150%, or between about 50% and about 125%,
v) the plant part has a decreased carbon:nitrogen content relative to the corresponding wild-type plant or part thereof of at least about 10%, at least about 25%, at least about 40%, between about 10% and about 50%, or between about 25% and about 50%,
vi) expression of one or more genes involved in photosynthesis is increased in the plant part relative to the corresponding wild-type plant part,
vii) the plant part has an increased carbon content relative to the corresponding wild-type plant part of at least about 10%, at least about 25%, at least about 50%, at least about 75%, at least about 100%, at least about 125%, at least about 150%, between about 10% and about 300%, between about 50% and about 250%, or between about 100% and about 200%,
viii) the plant part has an increased energy content in the plant part relative to the corresponding wild-type plant part of at least about 10%, at least about 25%, at least about 50%, at least about 75%, at least about 100%, at least about 125%, at least about 150%, at least about 200%, at least about 250%, between about 10% and about 400%, between about 50% and about 300%, or between about 200% and about 300%,
ix) the plant part has a decreased starch content relative to the corresponding wild-type plant part of at least about 2 fold, at least about 5 fold, at least about 10 fold, at least about 15 fold, at least about 20 fold, at least about 25 fold, between about 5 fold and about 35 fold, between about 10 fold and about 30 fold, or between about 20 fold and about 30 fold,
x) the plant part has a decreased TDF content relative to the corresponding wild-type plant part of at least about 10%, at least about 30%, at least about 50%, between about 10% and about 70%, or between about 30% and about 65%, and
xi) the plant part has a soluble sugar content relative to the corresponding wild-type plant part which is about 0.5 fold to 2 fold.

In an embodiment of the first and second aspects, plant part has one or more or all of;
i) the plant part comprises a total non-polar lipid content of at least about 8%, at least about 10%, at least about 11%, at least about 12%, at least about 15%, at least about 20%, at least about 25%, at least about 30%, at least about 35%, at least about 40%, at least about 45%, at least about 50%, at least about 55%, at least about 60%, at least about 65%, at least about 70%, between 8% and 75%, between 10% and 75%, between 11% and 75%, between about 15% and 75%, between about 20% and 75%, between about 30% and 75%, between about 40% and 75%, between about 50% and 75%, between about 60% and 75%, or between about 25% and 50% (w/w dry weight), preferably before flowering,
ii) the plant part is a vegetative part that comprises a TAG content of at least about 8%, at least about 10%, at least about 11%, at least about 12%, at least about 15%, at least about 20%, at least about 25%, at least about 30%, at least about 35%, at least about 40%, at least about 45%, at least about 50%, at least about 55%, at least about 60%, at least about 65%, at least about 70%, between 8% and 75%, between 10% and 75%, between 11% and 75%, between about 15% and 75%, between about 20% and 75%, between about 30% and 75%, between about 40% and 75%, between about 50% and 75%, between about 60% and 75%, or between about 25% and 50% (w/w dry weight), preferably before flowering,
iii) one or more or all of the promoters are selected from a tissue-specific promoter such as a leaf and/or stem specific promoter, a developmentally regulated promoter such as a senescence-specific promoter such as a SAG12 promoter, an inducible promoter, or a circadian-rhythm regulated promoter,
iv) the plant part is one member of a population or collection of at least about 1,500, at least about 3,000 or at least about 5,000 such plant parts, preferably vegetative plant parts.

In a further embodiment, the plant part is:
i) a 16:3 plant part, and which comprises one or more or all of the following:
   a) an exogenous polynucleotide which encodes a polypeptide which increases the export of fatty acids out of plastids of the plant when compared to a corresponding plant lacking the exogenous polynucleotide,
   b) a first genetic modification which down-regulates endogenous production and/or activity of a polypeptide involved in importing fatty acids into plastids of the plant when compared to a corresponding plant lacking the first genetic modification, and
   c) a second genetic modification which down-regulates endogenous production and/or activity of a polypeptide involved in diacylglycerol (DAG) production in the plastid when compared to a corresponding plant lacking the second genetic modification,
      wherein the exogenous polynucleotide is operably linked to a promoter which is capable of directing expression of the polynucleotide in the plant part, or
ii) a 18:3 plant part.

In an embodiment, the plant part has one or more or all of:
i) the plant part, preferably a vegetative plant part which has an increased synthesis of total fatty acids relative to a corresponding plant part lacking the exogenous polynucleotide(s) and/or genetic modification(s),
ii) the plant part, preferably a vegetative plant part which has an increased expression and/or activity of a fatty acyl acyltransferase which catalyses the synthesis of TAG, DAG or MAG, preferably TAG, relative to a corresponding plant part lacking the exogenous polynucleotide(s) and/or genetic modification(s),
iii) the plant part, preferably a vegetative plant part which has a decreased production of lysophosphatidic acid (LPA) from acyl-ACP and G3P in its plastids relative to a corresponding plant part lacking the exogenous polynucleotide(s) and/or genetic modification(s),
iv) the plant part, preferably a vegetative plant part which has an altered ratio of C16:3 to C18:3 fatty acids in its total fatty acid content and/or its galactolipid content relative to a corresponding part lacking the exogenous polynucleotide(s) and/or genetic modification(s), preferably a decreased ratio, v) one or more or all of the promoters are selected from promoter other than a constitutive promoter, preferably a tissue-specific promoter such as a leaf and/or stem specific promoter, a developmentally regulated promoter such as a senescense-specific promoter such as a SAG12 promoter, an inducible promoter, or a circadian-rhythm regulated promoter, preferably wherein at least one of the promoters operably linked to an exogenous polynucleotide which encodes a transcription factor polypeptide is a promoter other than a constitutive promoter,
vi) the plant part, preferably a vegetative plant part, comprises a total fatty acid content whose oleic acid level and/or palmitic acid level is increased by at least 2% relative to a corresponding plant, or part thereof, lacking the exogenous polynucleotide(s) and/or genetic modification(s), and/or whose α-linolenic acid (ALA) level and/or linoleic acid level is decreased by at least 2% relative to a corresponding plant part lacking the exogenous polynucleotide(s) and/or genetic modification(s),
vii) non-polar lipid in the plant part, preferably a vegetative plant part, comprises a modified level of total sterols, preferably free (non-esterified) sterols, steroyl esters, steroyl glycosides, relative to the non-polar lipid in a corresponding plant, or part thereof, lacking the exogenous polynucleotide(s) and/or genetic modification(s),
viii) non-polar lipid in the plant part comprises waxes and/or wax esters,
ix) the plant part comprises an exogenous polynucleotide encoding a silencing suppressor, wherein the exogenous polynucleotide is operably linked to a promoter which is capable of directing expression of the polynucleotide in the plant,
x) the level of one or more non-polar lipid(s) and/or the total non-polar lipid content of the plant or part thereof, preferably a vegetative plant part, is at least 2% greater on a weight basis than in a corresponding plant or part, respectively, which comprises exogenous polynucleotides encoding an Arabidposis thaliana WRI1 and an Arabidopsis thaliana DGAT1 (SEQ ID NO:1),
xi) a total polyunsaturated fatty acid (PUFA) content which is decreased relative to the total PUFA content of a corresponding plant lacking the exogenous polynucleotide(s) and/or genetic modification(s),
xii) if the plant part is a seed, the seed germinates at a rate substantially the same as for a corresponding wild-type seed or when sown in soil produces a plant whose seed germinate at a rate substantially the same as for corresponding wild-type seed, and
xiii) the plant is an algal plant such as from diatoms (bacillariophytes), green algae (chlorophytes), blue-green algae (cyanophytes), golden-brown algae (chrysophytes), haptophytes, brown algae or heterokont algae.

In an embodiment, the plant part comprises a first exogenous polynucleotide encoding a WRI1, a second exogenous polynucleotide encoding a DGAT or a PDAT, preferably a DGAT1, a third exogenous polynucleotide encoding an RNA which reduces expression of a gene encoding an SDP1 polypeptide, and a fourth exogenous polynucleotide encoding an oleosin. In preferred embodiments, the plant part has one or more or all of the following features:
i) a total lipid content of at least 8%, at least 10%, at least 12%, at least 14%, or at least 15.5% (% weight),
ii) at least a 3 fold, at least a 5 fold, at least a 7 fold, at least an 8 fold, or least a 10 fold, at higher total lipid content in the plant part relative to a corresponding the plant part lacking the exogenous polynucleotides and/or genetic modifications,
iii) a total TAG content of at least 5%, at least 6%, at least 6.5% or at least 7% (% weight of dry weight or seed weight),
iv) at least a 40 fold, at least a 50 fold, at least a 60 fold, or at least 70 fold, at least 100 fold, or at least a 120-fold higher total TAG content relative to a corresponding the plant part lacking the exogenous polynucleotides and/or genetic modifications,
v) palmitic acid comprises at least 20%, at least 25%, at least 30% or at least 33% (% weight) of the fatty acids in TAG,
vi) at least a 1.5 fold higher level of palmitic acid in TAG relative to a corresponding the plant part lacking the exogenous polynucleotides and/or genetic modifications,
vii) linoleic acid comprises at least 22%, at least 25%, at least 30% or at least 34% (% weight) of the fatty acids in TAG, and
viii) α-linolenic acid comprises less than 20%, less than 15%, less than 11% or less than 8% (% weight) of the fatty acids in TAG.
ix) at least a 5 fold, or at least an 8 fold, lower level of α-linolenic acid in TAG relative to a corresponding the plant or part thereof lacking the exogenous polynucleotides and/or genetic modifications, In the above embodiments, a preferred plant part is a leaf piece having a surface area of at least 1 cm² or a stem piece having a length of at least 1 cm.

In an embodiment of the above aspects, the plant part has been treated so it is no longer able to be propagated or give rise to a living plant, i.e. it is dead (for example a brown leaf or stem). For example, the plant part has been dried and/or ground. In another embodiment, the plant part is alive (for example, a green leaf or stem).

In an embodiment, the part is a seed, fruit, or a vegetative part such as an aerial plant part or a green part such as a leaf or stem.

In the above embodiments, it is preferred that the plant part is a vegetative plant part which is growing in soil or which was grown in soil and the plant part was subsequently harvested, and wherein the vegetative part comprises at least 8% TAG on a weight basis (% dry weight) such as for example between 8% and 75% or between 8% and 30%. More preferably, the TAG content is at least 10%, such as for example between 10% and 75% or between 10% and 30%. Preferably, these TAG levels are present in the vegetative parts prior to or at flowering of the plant or prior to seed setting stage of plant development. In these embodiments, it is preferred that the ratio of the TAG content in the leaves to the TAG content in the stems of the plant is between 1:1 and 10:1, and/or the ratio is increased relative to a corresponding vegetative part comprising the first and second exogenous polynucleotides and lacking the first genetic modification. Preferably, the vegetative plant part has an increased soluble protein content relative to the corresponding wild-type vegetative plant part of at least about 100%, or between about 50% and about 125%. Preferably, the vegetative plant part has an increased nitrogen content relative to the corresponding wild-type vegetative part of at least about 100%, or between about 50% and about 125%. Preferably, the vegetative plant part has an decreased carbon:nitrogen content relative to the corresponding wild-type vegetative part of at least about 40%, or between about 25% and about 50%. Preferably, the vegetative plant part has a decreased TDF content in the part or at least a part of the transgenic plant relative to the corresponding wild-type vegetative plant part of at least about 30%, or between about 30% and about 65%.

In an embodiment, the plant part, preferably a leaf, a grain, a stem, a root or an endosperm is from a monocotyledonous plant, which has a total fatty acid content or TAG content which is increased at least 5-fold on a weight basis when compared to a corresponding non-transgenic monocotyledonous plant. Alternatively, the transgenic monocotyledonous plant has endosperm comprising a TAG content which is at least 2.0%, preferably at least 3%, more preferably at least 4% or at least 5%, on a weight basis. In an embodiment, the endosperm has a TAG content of at least 2% which is increased at least 5-fold relative to a corresponding non-transgenic endosperm. Preferably, the plant is fully male and female fertile, its pollen is essentially 100% viable, and its grain has a germination rate which is between 70% and 100% relative to corresponding wild-type grain. In an embodiment, the transgenic plant is a progeny plant at least two generations derived from an initial transgenic wheat plant, and is preferably homozygous for the transgenes. In embodiments, the monocotyledonous plant, or part thereof, preferably a leaf, stem, grain or endosperm, is further characterised by one or more features as defined in the context of a plant or part thereof of the invention. In embodiments, the monocotyledonous plant, or part thereof, preferably a leaf, a grain, stem or an endosperm of the invention preferably has an increased level of monounsaturated fatty acids (MUFA) and/or a lower level of polyunsaturated fatty acids (PUFA) in both the total fatty acid content and in the TAG fraction of the total fatty acid content, such as for example an increased level of oleic acid and a reduced level of LA (18:2), when compared to a corresponding plant or part thereof lacking the genetic modifications and/or exogenous polynucleotide(s). Preferably, the linoleic acid (LA, 18:2) level in the total fatty acid content of the grain or endosperm of the monocotyledonous plant is reduced by at least 5% and/or the level of oleic acid in the total fatty acid content is increased by at least 5% relative to a corresponding wild-type plant or part thereof, preferably at least 10% or more preferably at least 15%, when compared to a corresponding plant or part thereof lacking the genetic modifications and/or exogenous polynucleotide(s).

In an embodiment of the first and second aspects, the extracted lipid is in the form of an oil, wherein at least about 90%, or least about 95%, at least about 98%, or between about 95% and about 98%, by weight of the oil is the lipid.

In an embodiment of the first and second aspects, the plant part is a vegetative plant part such as a plant leaf or stem, or the plant part is a seed or a fruit.

In an embodiment of the first and second aspects the plant part is from a species selected from a group consisting of a Acrocomia aculeata (macauba palm), Arabidopsis thaliana, Aracinis hypogaea (peanut), Astrocaryum murumuru (murumuru), Astrocaryum vulgare (tucumã), Attalea geraensis (Indaiá-rateiro), Attalea humilis (American oil palm), Attalea oleifera (andaiá), Attalea phalerata (uricuri), Attalea speciosa (babassu), Avena sativa (oats), Beta vulgaris (sugar beet), Brassica sp. such as Brassica carinata, Brassica juncea, Brassica napobrassica, Brassica napus (canola), Camelina sativa (false flax), Cannabis sativa (hemp), Carthamus tinctorius (safflower), Caryocar brasiliense (pequi), Cocos nucifera (Coconut), Crambe abyssinica (Abyssinian kale), Cucumis melo (melon), Elaeis guineensis (African palm), Glycine max (soybean), Gossypium hirsutum (cotton), Helianthus sp. such as Helianthus annuus (sunflower), Hordeum vulgare (barley), Jatropha curcas (physic nut), Joannesia princeps (arara nut-tree), Lemna sp. (duckweed) such as Lemna aequinoctialis, Lemna disperma, Lemna ecuadoriensis, Lemna gibba (swollen duckweed), Lemna japonica, Lemna minor, Lemna minuta, Lemna obscura, Lemna paucicostata, Lemna perpusilla, Lemna tenera, Lemna trisulca, Lemna turionifera, Lemna valdiviana, Lemna yungensis, Licania rigida (oiticica), Linum usitatissimum (flax), Lupinus angustifolius (lupin), Mauritia flexuosa (buriti palm), Maximiliana maripa (inaja palm), Miscanthus sp. such as Miscanthus x giganteus and Miscanthus sinensis, Nicotiana sp. (tabacco) such as Nicotiana tabacum or Nicotiana benthamiana, Oenocarpus bacaba (bacaba-do-azeite), Oenocarpus bataua (patauã), Oenocarpus distichus (bacaba-de-leque), Oryza sp. (rice) such as Oryza sativa and Oryza glaberrima, Panicum virgatum (switchgrass), Paraqueiba paraensis (mari), Persea amencana (avocado), Pongamia pinnata (Indian beech), Populus trichocarpa, Ricinus communis (castor), Saccharum sp. (sugarcane), Sesamum indicum (sesame), Solanum tuberosum (potato), Sorghum sp. such as Sorghum bicolor, Sorghum vulgare, Theobroma grandifor um (cupuassu), Trifolium sp., Trithrinax brasiliensis (Brazilian needle palm), Triticum sp. (wheat) such as Triticum aestivum and Zea mays (corn). For example, the plant part is from a monocotyledonous plant, preferably a plant from the family Poaceae, more preferably a Sorghum sp., a Zea mays, Miscanthus sp. such as Miscanthus x giganteus and Miscanthus sinensis, and/or a Panicum virgatum (switchgrass) plant.

In an embodiment of the first and second aspects, the one or more or all of the promoters are expressed at a higher level in a vegetative plant part relative to seed of a plant.

In another aspect, the present invention provides extracted plant lipid produced by the process of both the first and second aspects, preferably comprising plant leaf lipid.

In another aspect, the present invention provides extracted plant lipid, comprising fatty acids in an esterified form, wherein the level of medium chain fatty acids in the total fatty acid content of the lipid in the vegetative plant part is at least about 25%. In an embodiment, the lipid has one or more of the features defined above in relation to the first or second aspects.

In another aspect, the present invention provides a cell comprising an increased level or activity of polypeptides which are:
i. a GPAT, a LPAAT, and a WRI1 polypeptide;
   ii. a GPAT, a LPAAT, a DGAT and a WRI1 polypeptide;
   iii. a GPAT9, a LPAAT, and a WRI1 polypeptide;
   iv. a GPAT9, a LPAAT, a DGAT, and a WRI1 polypeptide;
   v. a GPAT, a LPAAT, a DGAT1, and a WRI1 polypeptide;
   vi. a GPAT9, a LPAAT, a DGAT1, and a WRI1 polypeptide;
   vii. a GPAT, a LPAAT, a WRI1 polypeptide, and a fatty acid thioesterase, preferably a FATB polypeptide;
   viii. a GPAT, a LPAAT, a DGAT, a WRI1 polypeptide, and a fatty acid thioesterase, preferably a FATB polypeptide;
   ix. a GPAT9, a LPAAT, a WRI1 polypeptide, and a fatty acid thioesterase, preferably a FATB polypeptide;
   x. a GPAT9, a LPAAT, a DGAT, a WRI1 polypeptide, and a fatty acid thioesterase, preferably a FATB polypeptide;
   xi. a GPAT, a LPAAT, a DGAT1, a WRI1 polypeptide, and a fatty acid thioesterase, preferably a FATB polypeptide;
   xii. a GPAT9, a LPAAT, a DGAT1, a WRI1 polypeptide, and a fatty acid thioesterase, preferably a FATB polypeptide;
   xiii. a GPAT, a LPAAT, a WRI1 polypeptide, a fatty acid thioesterase, preferably a FATB polypeptide, and an OBC polypeptide such as an oleosin, preferably a caleosin or a LDAP;
   xiv. a GPAT, a LPAAT, a DGAT, a WRI1 polypeptide, a fatty acid thioesterase, preferably a FATB polypeptide, and an OBC polypeptide such as an oleosin, preferably a caleosin or a LDAP;
   xv. a GPAT9, a LPAAT, a WRI1 polypeptide, a fatty acid thioesterase, preferably a FATB polypeptide, and an OBC polypeptide such as an oleosin, preferably a caleosin or a LDAP; a GPAT9, a LPAAT, a DGAT, a WRI1 polypeptide, a fatty acid thioesterase, preferably a xvi. FATB polypeptide, and an OBC polypeptide such as an oleosin, preferably a caleosin or a LDAP;
   xvii. a GPAT, a LPAAT, a DGAT1, a WRI1 polypeptide, a fatty acid thioesterase, preferably a FATB polypeptide, and an OBC polypeptide such as an oleosin, preferably a caleosin or a LDAP;
   xviii. a GPAT9, a LPAAT, a DGAT1, a WRI1 polypeptide, a fatty acid thioesterase, preferably a FATB polypeptide, and an OBC polypeptide such as an oleosin, preferably a caleosin or a LDAP;
   xix. a GPAT, a LPAAT, a WRI1 polypeptide, a fatty acid thioesterase, preferably a FATB polypeptide, and a silencing RNA which reduces the expression of an endogenous gene which encodes a SDP1 lipase;
   xx. a GPAT, a LPAAT, a DGAT, a WRI1 polypeptide, a fatty acid thioesterase, preferably a FATB polypeptide, and a silencing RNA which reduces the expression of an endogenous gene which encodes a SDP1 lipase;
   xxi. a GPAT9, a LPAAT, a WRI1 polypeptide, a fatty acid thioesterase, preferably a FATB polypeptide, and a silencing RNA which reduces the expression of an endogenous gene which encodes a SDP1 lipase;
   xxii. a GPAT9, a LPAAT, a DGAT, a WRI1 polypeptide, a fatty acid thioesterase, preferably a FATB polypeptide, and a silencing RNA which reduces the expression of an endogenous gene which encodes a SDP1 lipase;
   xxiii. a GPAT, a LPAAT, a DGAT1, a WRI1 polypeptide, a fatty acid thioesterase, preferably a FATB polypeptide, and a silencing RNA which reduces the expression of an endogenous gene which encodes a SDP1 lipase;
   xxiv. a GPAT9, a LPAAT, a DGAT1, a WRI1 polypeptide, a fatty acid thioesterase, preferably a FATB polypeptide, and a silencing RNA which reduces the expression of an endogenous gene which encodes a SDP1 lipase;
   xxv. a GPAT, a LPAAT, a DGAT, a WRI1 polypeptide, a fatty acid thioesterase, preferably a FATB polypeptide, an OBC polypeptide such as an oleosin, preferably a caleosin or a LDAP, and a silencing RNA which reduces the expression of an endogenous gene which encodes a SDP1 lipase;
   xxvi. a GPAT9, a LPAAT, a WRI1 polypeptide, a fatty acid thioesterase, preferably a FATB polypeptide, an OBC polypeptide such as an oleosin, preferably a caleosin or a LDAP, and a silencing RNA which reduces the expression of an endogenous gene which encodes a SDP1 lipase;
   xxvii. a GPAT9, a LPAAT, a DGAT, a WRI1 polypeptide, a fatty acid thioesterase, preferably a FATB polypeptide, an OBC polypeptide such as an oleosin, preferably a caleosin or a LDAP, and a silencing RNA which reduces the expression of an endogenous gene which encodes a SDP1 lipase;
   xxviii. a GPAT, a LPAAT, a DGAT1, a WRI1 polypeptide, a fatty acid thioesterase, preferably a FATB polypeptide, an OBC polypeptide such as an oleosin, preferably a caleosin, or a LDAP, and a silencing RNA which reduces the expression of an endogenous gene which encodes a SDP1 lipase; or
   xxix. a GPAT9, a LPAAT, a DGAT1, a WRI1 polypeptide, a fatty acid thioesterase, preferably a FATB polypeptide, an OBC polypeptide such as an oleosin, preferably a caleosin, or a LDAP, and a silencing RNA which reduces the expression of an endogenous gene which encodes a SDP1 lipase.

In an embodiment, the one or more or all of the polypeptides are encoded by one or more exogenous polynucleotides in the plant parts.

In an embodiment, the level of total, or new, MCFA is increased relative to a corresponding wild-type plant part, preferably at least 25% of the total fatty acid content on a weight basis.

In an embodiment, the one or more or all of the encoded GPAT, LPAAT and/or DGAT have a preference for utilising medium chain fatty acid substrates.

In an embodiment, the cell further comprises one or more or all of:
i. a genetic modification which down-regulates endogenous production and/or activity of a polypeptide involved in the catabolism of triacylglycerols (TAG) in the cell when compared to a corresponding cell lacking the genetic modification;
ii. a genetic modification which down-regulates endogenous production and/or activity of a polypeptide involved in importing fatty acids into plastids of the cell when compared to a corresponding cell lacking the genetic modification; and
iii. a genetic modification which down-regulates endogenous production and/or activity of a polypeptide involved in diacylglycerol (DAG) production in the plastid when compared to a corresponding cell lacking the genetic modification.

In an embodiment, the genetic modification is a mutation of an endogenous gene which partially or completely inactivates the gene, such as a point mutation, an insertion, or a deletion, or the genetic modification is an exogenous polynucleotide encoding an RNA molecule which inhibits expression of the endogenous gene, wherein the exogenous polynucleotide is operably linked to a promoter which is capable of directing expression of the polynucleotide in the cell.

In an embodiment, the one or more or all of the promoters are expressed at a higher level in a vegetative plant part relative to seed of a plant.

In an embodiment, the cell has one or more or all of the following features:
i. the level of medium chain fatty acids in the total fatty acid content of the cell, and/or in the total fatty acid content of the TAG of the cell, is at least about 30%, at least about 35%, at least about 40%, at least about 50%, at least about 55%, or between about 25% and about 55%, between about 25% and about 50%, between about 30% and about 50%, between about 35% and about 50%, between about 25% and about 40%, or between about 30% and about 40%;
ii. the level of lauric acid (C12:0) in the total fatty acid content of the cell, and/or in the total fatty acid content of the TAG of the cell, is, or is increased by, at least about 15%, at least about 20%, at least about 25%, at least about 30%, at least about 35%, at least about 40%, at least about 50%, at least or about 55%, or between about 15% and about 55%, between about 20% and about 50%, between about 30% and about 50%, between about 35% and about 50%, between about 15% and about 25%, or between about 20% and about 30%;
iii the level of myristic acid (C14:0) in the total fatty acid content of the cell, and/or in the total fatty acid content of the TAG of the cell, is, or is increased by, at least about 25%, at least about 30%, at least about 35%, at least about 40%, at least about 45%, or between about 25% and about 45%, between about 20% and about 50%, between about 30% and about 50%, between about 35% and about 50%, between about 30% and about 40%, between about 15% and about 25%, or between about 20% and about 30%;
iv. the level of palmitic acid (C16:0) in the total fatty acid content of the cell, and/or in the total fatty acid content of the TAG of the cell, is, or is increased by, between about 2% and about 18%, or between about 2% and about 16%, or between about 2% and about 15%, or between about 15% and about 50%;
v. the level of lauric acid (C12:0) in the total fatty acid content of the cell, and/or in the total fatty acid content of the TAG of the cell is, or is increased by, at least about 25%, at least about 30%, at least about 40%, at least about 45%, or at least about 50%, and the level of myristic acid (C14:0) in the total fatty acid content of the cell and/or in the total fatty acid content of the TAG of the cell is, or is increased by, at least about 1%, at least about 2%, at least about 5%, or at least about 10%, or between about 1% and about 10%, or between about 2% and 10%, or between about 2% and about 6%, or less than about 10%, or less than about 8% or less than about 5%, or less than about 2%;
vi. the level of myristic acid (C14:0) in the total fatty acid content of the cell, and/or in the total fatty acid content of the TAG of the cell is, or is increased by, at least about 20%, at least about 25%, at least about 30%, or at least about 40%, and the level of lauric acid (C12:0) in the total fatty acid content of the cell and/or in the total fatty acid content of the TAG of the cell is, or is increased by, at least about 1%, at least about 2%, at least about 5%, or at least about 10%, or between about 1% and about 10%, or between about 2% and about 10%, or between about 2% and about 6%, or less than about 10%, or less than about 8% or less than about 5%, or less than about 2%;
vii. the level of myristic acid (C14:0) in the total fatty acid content of the cell, and/or in the total fatty acid content of the TAG of the cell is, or is increased by, at least about 20%, at least about 25%, at least about 30%, and the level of palmitic acid (C16:0) in the total fatty acid content of the cell and/or in the total fatty acid content of the TAG of the cell is, or is increased by, at least about 2%, at least about 3%, at least about 4%, or at least about 5%.
viii. the ratio of lauric acid (C12:0): myristic acid (C14:0) in the fatty acid content of the cell, and/or in the total fatty acid content of the TAG of the cell, is increased, or is about 1:4, about 1:5, about 1:10, about 1:15, about 1:20, about 1:25, or about 4:1, about 5:1, about 10:1, about 15:1, about 20:1, about 30:1, about 40:1, or about 45:1;
ix. the ratio of lauric acid (C12:0):palmitic acid (C16:0) in the fatty acid content of the cell, and/or in the total fatty acid content of the TAG of the cell, is increased, or is about 1:2, about 1:3, about 1:4, about 1:5, about 1:10, about 1:15, about 10:1, about 20:1, about 30:1, about 40:1, or about 45:1;
x. the ratio of myristic acid (C14:0):palmitic acid (C16:0) in the fatty acid content of the cell, and/or in the total fatty acid content of the TAG of the cell, is increased, or is about 1:2, about 1:3, about 1:4, about 1:5, about 1:10, about 1:15, about 10:1, about 20:1, about 30:1, or about 40:1;
xi. the level of oleic acid in the total fatty acid content of the cell, and/or in the total fatty acid content of the TAG of the cell, is decreased, or is less than about 10%, less than about 8%, less than about 6%, less than about 5%, less than about 4%, less than about 3%, less than about 2%, less than about 1%;
xii. the level of linoleic acid (LA) in the total fatty acid content of the cell, and/or in the total fatty acid content of the TAG of the cell, is increased or decreased, or is less than about 20%, less than about 15%, less than about 10%, less than about 5%, less than about 4%, less than about 3%, less than about 2%, or less than about 1%;
xiii. the level of α-linolenic acid (ALA) in the total fatty acid content of the cell, or in the total fatty acid content of the TAG of the cell, is decreased or is less than about 50%, less than about 40%, less than about 30%, less than about 20%, less than about 10%, less than about 8%, less than about 6%, less than about 5%, less than about 2%, or less than about 1%;
xiv. the level of total unsaturated fatty acids in the total fatty acid content of the cell, and/or in the total fatty acid content of the TAG of the cell, is decreased, or is less than about 50%, less than about 40%, less than about 30%, less than about 20%, less than about 15%, less than about 10%, less than about 8%, less than about 6%, less than about 5%, less than about 2%, or less than about 1%;
xv. the level of total monounsaturated fatty acids in the total fatty acid content of the cell, and/or in the total fatty acid content of the TAG of the cell, is decreased, or is less than about 10%, less than about 5%, less than about 4%, less than about 3%, less than about 2%, or less than about 1%;
xvi. the level of total polyunsaturated fatty acids in the total fatty acid content of the cell, and/or in the total fatty acid content of the TAG of the cell, is less than about 50%, less than about 40%, less than about 30%, less than about 20%, less than about 15%, less than about 10%, less than about 8%, less than about 6%, less than about 5%, less than about 2%, or less than about 1%;
xvii. the triacylglycerol (TAG) content of the cell is at least about 80%, at least about 85%, at least about 90%, or least about 95%, and about 98%, or between about 95% and about 98%, by weight of the cell;
xviii. the TAG content of the cell comprises, or is increased in a level of, one or more or all of the TAG species 36:0, 38:0, 40:0 and 42:0;
xix. the cell comprises tri-laurin (tri-C12:0) and/or tri-myristin (tri-C14:0);
xx. the phosphocholine (PC) content of the cell comprises one or both of the PC species 28:0 and 30:0,
xxi. the cell has a reduced level of medium chain fatty acids, preferably C14:0, in membrane lipids relative to a corresponding cell;
xxii. the cell has less chlorosis relative to a corresponding cell which comprises the exogenous polynucleotide encoding the thioesterase but lacks the exogenous polynucleotide encoding the DGAT; and
xxiii. the cell is in a vegetative plant part and the part has an alleviated chlorosis phenotype relative to a corresponding vegetative plant part,
   wherein any increase or decrease is relative to a corresponding wild-type cell.

In another aspect, the present invention provides a plant or a part thereof comprising the cell of the invention, or which is transgenic for one or more exogenous polynucleotides defined above.

In an embodiment, before the plant flowers, a vegetative part of the plant comprises a total non-polar lipid content of at least about 8%, at least about 10%, about 11%, between 8% and 15%, or between 9% and 12% (w/w dry weight).

In an embodiment, the plant is a monocotyledonous plant, or part thereof preferably a leaf, a grain, a stem, a root or an endosperm, which has a total fatty acid content or TAG content which is increased at least 5-fold on a weight basis when compared to a corresponding non-transgenic monocotyledonous plant, or part thereof. Alternatively, the transgenic monocotyledonous plant has endosperm comprising a TAG content which is at least 2.0%, preferably at least 3%, more preferably at least 4% or at least 5%, on a weight basis, or part of the plant, preferably a leaf, a stem, a root, a grain or an endosperm. In an embodiment, the endosperm has a TAG content of at least 2% which is increased at least 5-fold relative to a corresponding non-transgenic endosperm. Preferably, the plant is fully male and female fertile, its pollen is essentially 100% viable, and its grain has a germination rate which is between 70% and 100% relative to corresponding wild-type grain. In an embodiment, the transgenic plant is a progeny plant at least two generations derived from an initial transgenic wheat plant, and is preferably homozygous for the transgenes. In embodiments, the monocotyledonous plant, or part thereof, preferably a leaf, stem, grain or endosperm, is further characterised by one or more features as defined in the context of a plant or part thereof of the invention. In embodiments, the monocotyledonous plant, or part thereof preferably a leaf, a grain, stem or an endosperm of the invention preferably has an increased level of monounsaturated fatty acids (MUFA) and/or a lower level of polyunsaturated fatty acids (PUFA) in both the total fatty acid content and in the TAG fraction of the total fatty acid content, such as for example an increased level of oleic acid and a reduced level of LA (18:2), when compared to a corresponding plant or part thereof lacking the genetic modifications and/or exogenous polynucleotide(s). Preferably, the linoleic acid (LA, 18:2) level in the total fatty acid content of the grain or endosperm of the monocotyledonous plant is reduced by at least 5% and/or the level of oleic acid in the total fatty acid content is increased by at least 5% relative to a corresponding wild-type plant or part thereof, preferably at least 10% or more preferably at least 15%, when compared to a corresponding plant or part thereof lacking the genetic modifications and/or exogenous polynucleotide(s).

In an embodiment, the plant, or part thereof, is a member of a population or collection of at least about 1,500, at least about 3,000 or at least about 5,000 such plants or parts.

In an embodiment, the TFA content, the TAG content, the total non-polar lipid content, or the one or more non-polar lipids, and/or the level of the oleic acid or a PUFA in the plant or part thereof is determinable by analysis by using gas chromatography of fatty acid methyl esters obtained from the plant or vegetative part thereof.

In a further embodiment, wherein the plant part is a leaf and the total non-polar lipid content of the leaf is determinable by analysis using Nuclear Magnetic Resonance (NMR).

In each of the above embodiments, it is preferred that the plant is a transgenic progeny plant at least two generations derived from an initial transgenic plant, and is preferably homozygous for the transgenes.

In an embodiment, the plant or the part thereof is phenotypically normal, in that it is not significantly reduced in its ability to grow and reproduce when compared to an unmodified plant or part thereof. In an embodiment, the biomass, growth rate, germination rate, storage organ size, seed size and/or the number of viable seeds produced is not less than 70%, not less than 80% or not less than 90% of that of a corresponding wild-type plant when grown under identical conditions. In an embodiment, the plant is male and female fertile to the same extent as a corresponding wild-type plant and its pollen (if produced) is as viable as the pollen of the corresponding wild-type plant, preferably at least about 75%, or at least about 90%, or close to 100% viable. In an embodiment, the plant produces seed which has a germination rate of at least about 75% or at least about 90% relative to the germination rate of corresponding seed of a wild-type plant, where the plant species produces seed. In an embodiment, the plant of the invention has a plant height which is at least about 75%, or at least about 90% relative to the height of the corresponding wild-type plant grown under the same conditions. A combination of each of these features is envisaged. In an alternative embodiment, the plant of the invention has a plant height which is between 60% and 90% relative to the height of the corresponding wild-type plant grown under the same conditions. In an embodiment, the plant or part thereof of the invention, preferably a plant leaf, does not exhibit increased necrosis, i.e. the extent of necrosis, if present, is the same as that exhibited by a corresponding wild-type plant or part thereof grown under the same conditions and at the same stage of plant development. This feature applies in particular to the plant or part thereof comprising an exogenous polynucleotide which encodes a fatty acid thioesterase such as a FATB thioesterase.

In another aspect, the present invention provides a population of at least about 1,500, at least about 3,000 or at least about 5,000 plants, each being a plant of the invention, growing in a field.

In an embodiment, the exogenous polynucleotides are inserted at the same chromosomal location in the genome of each of the plants, preferably in the nuclear genome of each of the plants.

In another aspect, the present invention provides a population of at least about 1000 plants, each being a plant according to the invention, growing in a field, or a collection of at least about 1000 plant parts, each being a plant part according to the invention, wherein the plant parts have been harvested from plants growing in a field.

Also provided is a storage bin comprising a collection of plants or plant parts of the invention. In another aspect, the present invention provides an extract of a plant or a part thereof of the invention. The extract preferably has a different fatty acid composition relative to a corresponding wild-type extract.

In an embodiment, the extract is lacking at least 50% or at least 90% of the chlorophyll and/or soluble sugars of the plant or part thereof.

In a further aspect, the present invention provides a process for selecting a plant or a part thereof with a desired phenotype, the process comprising
i) obtaining a plurality of candidate plants, or parts thereof, which each comprise
   a) a first exogenous polynucleotide which encodes a transcription factor polypeptide that increases the expression of one or more glycolytic and/or fatty acid biosynthetic genes in a plant or part thereof, and
   b) a second exogenous polynucleotide which encodes a polypeptide involved in the biosynthesis of one or more non-polar lipids, wherein each exogenous polynucleotide is operably linked to a promoter which is capable of directing expression of the polynucleotide in the plant, or part thereof,
ii) analysing lipid in the plurality of parts, or at least a part of each plant in the plurality of candidate plants, from step i),
iii) analysing the plurality of parts, or at least a part of each plant in the plurality of candidate plants, from step i) for one or more or all of;
   a) soluble protein content,
   b) nitrogen content,
   c) carbon:nitrogen ratio,
   d) photosynthetic gene expression,
   e) photosynthetic capacity,
   f) total dietary fibre (TDF) content,
   g) carbon content, and
   h) energy content, and
iv) selecting a plant or part thereof which comprises an increased triacylglycerol (TAG) content in the part or at least a part of the plant relative to a corresponding wild-type plant or part thereof and a desired phenotype selected from one or more or all of the following;
   A) an increased soluble protein content in the part or at least a part of the plant relative to a corresponding wild-type plant or part thereof,
   B) an increased nitrogen content in the part or at least a part of the plant relative to a corresponding wild-type plant or part thereof,
   C) decreased carbon:nitrogen ratio in the part or at least a part of the plant relative to a corresponding wild-type plant or part thereof,
   D) increased photosynthetic gene expression in the part or at least a part of the plant relative to a corresponding wild-type plant or part thereof,
   E) increased photosynthetic capacity in the part or at least a part of the plant relative to a corresponding wild-type plant or part thereof,
   F) decreased total dietary fibre (TDF) content in the part or at least a part of the plant relative to a corresponding wild-type plant or part thereof,
   G) increased carbon content in the part or at least a part of the plant relative to a corresponding wild-type plant or part thereof, and
   H) increased energy content in the part or at least a part of the plant relative to a corresponding wild-type plant or part thereof.

In an embodiment, the process further comprises a step of obtaining seed or a progeny plant from the transgenic plant, wherein the seed or progeny plant comprises the exogenous polynucleotides.

In an embodiment, the increased triacylglycerol (TAG) content is determined by analysing one or more of the total fatty acid content, TAG content, fatty acid composition, by any means, which might or might not involve first extracting the lipid.

In yet another embodiment, the selected plant or part thereof has one or more of the features as defined herein.

In another aspect, the present invention provides seed of, or obtained from, a plant according to the invention.

In another aspect, the present invention provides a process for obtaining a cell according to the invention, the process comprising the steps of:
i) introducing into a cell at least one exogenous polynucleotide and/or at least one genetic modification as defined above to produce a cell as defined above,
ii) expressing the exogenous polynucleotide(s) in the cell or a progeny cell thereof,
iii) analysing the lipid content of the cell or progeny cell, and
iv) selecting a cell according to the invention.

In another aspect, the present invention provides a method of producing a plant which has integrated into its genome a set of exogenous polynucleotides and/or genetic modifications as defined above, the method comprising the steps of:
i) crossing two parental plants, wherein one plant comprises at least one of the exogenous polynucleotides and/or at least one genetic modifications as defined in any one of claims 24 to 31; and the other plant comprises at least one of the exogenous polynucleotides and/or at least one genetic modifications as defined in any one of claims 24 to 31, and wherein between them the two parental plants comprise a set of exogenous polynucleotides and/or genetic modifications as defined in any one of claims 24 to 31,
ii) screening one or more progeny plants from the cross for the presence or absence of the set of exogenous polynucleotides and/or genetic modifications as defined in any one of claims 24 to 31, and
iii) selecting a progeny plant which comprise the set of exogenous polynucleotides and/or genetic modifications as defined in any one of claims 24 to 31, thereby producing the plant.

In another aspect, the present invention provides a process for producing an industrial product, the process comprising the steps of:
i) obtaining a cell of the invention, a plant or a part thereof of the invention, or seed the invention, and
ii) either
   a) converting at least some of the lipid in the cell, plant or part thereof, or seed, of step i) to the industrial product by applying heat, chemical, or enzymatic means, or any combination thereof, to the lipid in situ in the cell, or plant or vegetative part thereof, or seed, or
   b) physically processing the cell, plant or part thereof, or seed, of step i), and subsequently or simultaneously converting at least some of the lipid in the processed cell, plant or part thereof, or seed, to the industrial product by applying heat, chemical, or enzymatic means, or any combination thereof, to the lipid in the processed cell, plant or part thereof, or seed, and
iii) recovering the industrial product, thereby producing the industrial product.

In an embodiment, the step of physically processing the the cell, plant or part thereof, or seed, of step i), comprises one or more of rolling, pressing, crushing or grinding the plant or part thereof, or seed.

In an embodiment, the invention further comprises steps of:
(a) extracting at least some of the non-polar lipid content of the cell, or plant or part thereof, or seed, as non-polar lipid, and
(b) recovering the extracted non-polar lipid, wherein steps (a) and (b) are performed prior to the step of converting at least some of the lipid in the cell, plant or part thereof, or seed, to the industrial product.

In another aspect, the present invention provides a process for producing extracted lipid, the process comprising the steps of:
i) obtaining a plant cell of the invention, or a plant or a part thereof of the invention, or seed of the invention,
ii) extracting lipid from the cell, or plant or part thereof, or seed, and
iii) recovering the extracted lipid, thereby producing the extracted lipid.

In an embodiment, a process of extraction comprises one or more of drying, rolling, pressing crushing or grinding the plant or part thereof, or seed, and/or purifying the extracted lipid or seedoil.

In an embodiment, the process uses an organic solvent in extraction process to extract the oil.

In an embodiment, the process comprises recovering the extracted lipid by collecting it in a container and/or one or more of degumming, deodorising, decolourising, drying, fractionating the extracted lipid, removing wax esters from the extracted lipid, or analysing the fatty acid composition of the extracted lipid.

In an embodiment, the volume of the extracted lipid or oil is at least 1 litre.

In a further embodiment, one or more or all of the following features apply:
(i) the extracted lipid or oil comprises triacylglycerols, wherein the triacylglycerols comprise at least 90%, preferably at least 95% or at least 96%, of the extracted lipid or oil,
(ii) the extracted lipid or oil comprises free sterols, steroyl esters, steroyl glycosides, waxes or wax esters, or any combination thereof, and
(iii) the total sterol content and/or composition in the extracted lipid or oil is significantly different to the sterol content and/or composition in the extracted lipid or oil produced from a corresponding plant or part thereof, or seed.

In an embodiment, the process further comprises converting the extracted lipid to an industrial product.

In an embodiment, the industrial product is a hydrocarbon product such as fatty acid esters, preferably fatty acid methyl esters and/or a fatty acid ethyl esters, an alkane such as methane, ethane or a longer-chain alkane, a mixture of longer chain alkanes, an alkene, a biofuel, carbon monoxide and/or hydrogen gas, a bioalcohol such as ethanol, propanol, or butanol, biochar, or a combination of carbon monoxide, hydrogen and biochar.

In a further embodiment, the plant part is an aerial plant part or a green plant part, preferably a vegetative plant part such as a plant leaf or stem.

In yet a further embodiment, the process further comprises a step of harvesting the plant or part thereof such as a vegetative plant part, tuber or beet, or seed, preferably with a mechanical harvester. In another embodiment, the level of a lipid in the plant or part thereof, or seed and/or in the extracted lipid or oil is determinable by analysis by using gas chromatography of fatty acid methyl esters prepared from the extracted lipid or oil.

In yet another embodiment, the process further comprises harvesting the part from a plant.

In an embodiment, the plant part is a vegetative plant part which comprises a total non-polar lipid content of at least about 18%, at least about 20%, at least about 25%, at least about 30%, at least about 35%, at least about 40%, at least about 45%, at least about 50%, at least about 55%, at least about 60%, at least about 65%, at least about 70%, between 18% and 75%, between about 20% and 75%, between about 30% and 75%, between about 40% and 75%, between about 50% and 75%, between about 60% and 75%, or between about 25% and 50% (w/w dry weight).

In a further embodiment, the plant part is a vegetative plant part which comprises a total TAG content of at least about 18%, at least about 20%, at least about 25%, at least about 30%, at least about 35%, at least about 40%, at least about 45%, at least about 50%, at least about 55%, at least about 60%, at least about 65%, at least about 70%, between 18% and 75%, between about 20% and 75%, between about 30% and 75%, between about 40% and 75%, between about 50% and 75%, between about 60% and 75%, or between about 25% and 50% (w/w dry weight).

In another embodiment, the plant part is a vegetative plant part which comprises a total non-polar lipid content of at least about 11%, at least about 12%, at least about 15%, at least about 20%, at least about 25%, at least about 30%, at least about 35%, at least about 40%, at least about 45%, at least about 50%, at least about 55%, at least about 60%, at least about 65%, at least about 70%, between 8% and 75%, between 10% and 75%, between 11% and 75%, between about 15% and 75%, between about 20% and 75%, between about 30% and 75%, between about 40% and 75%, between about 50% and 75%, between about 60% and 75%, or between about 25% and 50% (w/w dry weight), and wherein the vegetative plant part is from a 16:3 plant.

In yet another embodiment, the plant part is a vegetative plant part which comprises a total TAG content of at least about 11%, at least about 12%, at least about 15%, at least about 20%, at least about 25%, at least about 30%, at least about 35%, at least about 40%, at least about 45%, at least about 50%, at least about 55%, at least about 60%, at least about 65%, at least about 70%, between 8% and 75%, between 10% and 75%, between 11% and 75%, between about 15% and 75%, between about 20% and 75%, between about 30% and 75%, between about 40% and 75%, between about 50% and 75%, between about 60% and 75%, or between about 25% and 50% (w/w dry weight), and wherein the vegetative plant part is from a 16:3 plant.

In an embodiment, the vegetative plant parts have a TAG/TFA Quotient (TTQ) of between 0.01 and 0.6. In an embodiment, the vegetative plant parts have a TTQ of between 0.01 and 0.55, or between 0.01 and 0.5, or about 0.1, or about 0.2 or about 0.3, or about 0.4 or about 0.5. Preferably, the TTQ is between 0.60 and 0.84, which corresponds to a TAG:TFA ratio of between 1.5:1 and 5:1, or between 0.84 and 0.95 which corresponds to a TAG:TFA ratio of between 5:1 and 20:1.

In an embodiment, the vegetative plant parts comprise an average TFA content of about 6%, or about 8%, or about 9% or about 10% (w/w dry weight).

In an embodiment, the TFA content of the vegetative plant parts comprises a palmitic acid content which is increased by at least 2% or at least 3% relative to the palmitic acid content of a corresponding wild-type vegetative plant part.

In an embodiment, the TFA content of the vegetative plant parts comprises a «-linoleic acid (ALA) content which is decreased by at least 2% or at least 3% relative to the ALA content of a corresponding wild-type vegetative plant part.

In an embodiment, one or more or all of the following features apply:
(i) the vegetative plant parts are leaves and/or stems or parts thereof which comprise one or more of an increased carbon content, an increased energy content, an increased soluble protein content, a reduced starch content, a reduced total dietary fibre (TDF) content and an increased nitrogen content, each on a weight basis relative to a corresponding wild-type leaf or stem or parts thereof from a wild-type Sorghum sp. or Zea mays plant at the same stage of growth,
(ii) the TFA content of the vegetative plant parts is at least about 6%, at least about 7%, at least about 8%, at least about 9%, at least about 10%, at least about 11%, at least about 12%, at least about 15%, at least about 20%, at least about 25%, at least about 30%, at least about 35%, at least about 40%, at least about 45%, at least about 50%, at least about 55%, at least about 60%, at least about 65%, at least about 70%, between about 6% and about 20%, between 8% and 75%, between 10% and 75%, between 11% and 75%, between about 15% and 75%, between about 20% and 75%, between about 30% and 75%, between about 40% and 75%, between about 50% and 75%, between about 60% and 75%, or between about 25% and 50% (w/w dry weight) TFA,
(iii) the fatty acids esterified in the form of TAG in the vegetative plant parts is at least about 1%, at least about 2%, at least about 3%, at least about 4%, at least about 5%, at least about 6%, at least about 7%, at least about 8%, at least about 9%, at least about 10%, at least about 11%, at least about 12%, at least about 15%, at least about 20%, at least about 25%, at least about 30%, at least about 35%, at least about 40%, at least about 45%, at least about 50%, at least about 55%, at least about 60%, at least about 65%, at least about 70%, between about 6% and about 20%, between 8% and 75%, between 10% and 75%, between 11% and 75%, between about 15% and 75%, between about 20% and 75%, between about 30% and 75%, between about 40% and 75%, between about 50% and 75%, between about 60% and 75%, or between about 25% and 50% (w/w dry weight),
(iv) the vegetative plant parts comprise an increased content of a WRI1 polypeptide, an increased content of a DGAT polypeptide, and a decreased content of a SDP1 polypeptide, each relative to a corresponding wild-type vegetative plant part,
(v) the vegetative plant parts comprise an increased content of a WRI1 polypeptide, an increased content of a DGAT polypeptide, and an increased content of a LEC2 polypeptide, each relative to a corresponding wild-type vegetative plant part,
(vi) the vegetative plant parts comprise an increased content of a PDAT or DGAT polypeptide, a decreased content of a TGD polypeptide, and a decreased content of a SDP1 polypeptide, each relative to a corresponding wild-type vegetative plant part, and
(vii) the vegetative plant parts comprise a decreased content of a TAG lipase such as a SDP1 TAG lipase, a decreased content of a TGD polypeptide such as a TGD5 polypeptide, and optionally a decreased content of a TST polypeptide such as a TST1 polypeptide, each decrease being relative to a corresponding wild-type vegetative plant part.

In an embodiment, one or more or all of the following features apply:
(i) the vegetative plant parts are harvested from the plant between the time of first flowering of the plant and first maturity of seed,
(ii) the Sorghum sp. plant is a Sorghum bicolor plant,
(iii) the vegetative plant parts include leaves and/or stems or parts thereof,
(iv) the vegetative plant parts comprise an average total fatty acid content of about 8% or about 10% (w/w dry weight),

In another aspect, the present invention provides a process for producing seed, the process comprising:
i) growing a plant according to the invention, and
ii) harvesting seed from the plant.

In an embodiment, the above process comprises growing a population of at least about 1,500, at least about 3,000 or at least about 5,000 plants, each being a plant of the invention, and harvesting seed from the population of plants.

In another aspect, the present invention provides recovered or extracted lipid obtainable from a cell according to the invention, a plant or a part thereof of the invention, seed of the invention, or obtainable by the process of the invention.

In another aspect, the present invention provides an industrial product produced by the process according to the invention, which is a hydrocarbon product such as fatty acid esters, preferably fatty acid methyl esters and/or a fatty acid ethyl esters, an alkane such as methane, ethane or a longer-chain alkane, a mixture of longer chain alkanes, an alkene, a biofuel, carbon monoxide and/or hydrogen gas, a bioalcohol such as ethanol, propanol, or butanol, biochar, or a combination of carbon monoxide, hydrogen and biochar. In an embodiment the industrial product comprises MCFA, preferably an increased level of MCFA relative to a corresponding industrial product produced from a wild-type plant or part thereof.

In a further aspect, the present invention provides for the use of a transgenic plant or part thereof of the invention, seed of the invention, extract of the invention or the recovered or extracted lipid or soluble protein of the invention for the manufacture of an industrial product.

Examples of industrial products of the invention include, but are not limited to, a hydrocarbon product such as fatty acid esters, preferably fatty acid methyl esters and/or a fatty acid ethyl esters, an alkane such as methane, ethane or a longer-chain alkane, a mixture of longer chain alkanes, an alkene, a biofuel, carbon monoxide and/or hydrogen gas, a bioalcohol such as ethanol, propanol, or butanol, biochar, or a combination of carbon monoxide, hydrogen and biochar.

In another aspect, the present invention provides use of a cell according to the invention, a plant or part thereof of the invention, seed of the invention, or the lipid of the invention, for the manufacture of an industrial product.

In another aspect, the present invention provides a process for producing fuel, the process comprising:
i) reacting the lipid of the invention with an alcohol, optionally, in the presence of a catalyst, to produce alkyl esters, and
ii) optionally, blending the alkyl esters with petroleum based fuel.

In another aspect, the present invention provides a process for producing a synthetic diesel fuel, the process comprising:
i) converting the lipid in a cell of the invention, or a plant or a part thereof of the invention, or seed of the invention, to a bio-oil by a process comprising pyrolysis or hydrothermal processing or to a syngas by gasification, and
ii) converting the bio-oil to synthetic diesel fuel by a process comprising fractionation, preferably selecting hydrocarbon compounds which condense between about 150° C. to about 200° C. or between about 200° C. to about 300° C., or converting the syngas to a biofuel using a metal catalyst or a microbial catalyst.

In another aspect, the present invention provides a process for producing a biofuel, the process comprising converting the lipid in a cell of the invention, a plant or a part thereof of the invention, or seed of the invention, to bio-oil by pyrolysis, a bioalcohol by fermentation, or a biogas by gasification or anaerobic digestion.

In another aspect, the present invention provides a process for producing a feedstuff, the process comprising admixing a plant cell of the invention, a plant or a part thereof of the invention, seed of the invention, or the lipid of any one of claims the invention, or an extract or portion thereof, with at least one other food ingredient.

In another aspect, the present invention provides feedstuffs, cosmetics or chemicals comprising a plant cell of the invention, a plant or a part thereof of the invention, seed of the invention, or the lipid of the invention, or an extract or portion thereof.

In an embodiment, the feedstuff is silage, pellets or hay.

In another aspect, the present invention provides a process for feeding an animal, the process comprising providing to the animal a plant or a part thereof of the invention, seed of the invention, or the lipid of the invention.

In an embodiment, the animal ingests an increased amount of MCFA, nitrogen, protein, carbon and/or energy potential relative to when the animal ingests the same amount on a dry weight basis of a corresponding wild-type plant or part thereof, seed or extract or feedstuff produced from the corresponding wild-type plant or part thereof.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. A representation of lipid synthesis in eukaryotic cells, showing export of some of the fatty acids synthesized in the plastids to the Endoplasmic Reticulum (ER) via the Plastid Associated Membrane (PLAM), and import of some of the fatty acids into the plastid from the ER for eukaryotic galactolipid synthesis. Abbreviations:
Acetyl-CoA and Malonyl-CoA: acetyl-coenzyme A and malonyl-coenzymeA;
ACCase: Acetyl-CoA carboxylase;
FAS: fatty acid synthase complex;
16:0-ACP, 18:0-ACP and 18:1-ACP: C16:O-acyl carrier protein (ACP), C18:O-acyl carrier protein, C18:1-acyl carrier protein;
KAS II: ketoacyl-ACP synthase II (EC 2.3.1.41);
PLPAAT: plastidial LPAAT;
PGPAT: plastidial GPAT;
PAP: PA phosphorylase (EC 3.1.3.4);
G3P: glycerol-3-phosphate;
LPA: lysophosphatidic acid;
PA: phosphatidic acid;
DAG: diacylglycerol;
TAG: triacylglycerol;
Acyl-CoA and Acyl-PC: acyl-coenzyme A and acyl-phosphatidylcholine;
PC: phosphatidylcholine;
GPAT: glycerol-3-phosphate acyltransferase;
LPAAT: lysophosphatidic acid acyltransferase (EC 2.3.1.51);
LPCAT: acyl-CoA:lysophosphatidylcholine acyltransferase; or synonyms 1-acylglycerophosphocholine O-acyltransferase; acyl-CoA: 1-acyl-sn-glycero-3-phosphocholine O-acyltransferase (EC 2.3.1.23);
CPT: CDP-choline:diacylglycerol cholinephosphotransferase; or synonyms 1-alkyl-2-acetylglycerol cholinephosphotransferase; alkylacylglycerol cholinephosphotransferase; cholinephosphotransferase; phosphorylcholine-glyceride transferase (EC 2.7.8.2);
PDCT: phosphatidylcholine:diacylglycerol cholinephosphotransferase;
PLC: phospholipase C (EC 3.1.4.3);
PLD: Phospholipase D; choline phosphatase; lecithinase D; lipophosphodiesterase II (EC 3.1.4.4);
PDAT: phospholipid:diacylglycerol acyltransferase; or synonym phospholipid: 1,2-diacyl-sn-glycerol O-acyltransferase (EC 2.3.1.158);
FAD2: fatty acid A12-desaturase; FAD3, fatty acid 415-desaturase;
UDP-Gal: Uridine diphosphate galactose;
MGDS: monogalactosyldiacylglycerol synthase;
MGDG: monogalactosyldiacylglycerol; DGDG: digalactosyldiacylglycerol
FAD6, 7, 8: plastidial fatty acid Δ12-desaturase, plastidial ω3-desaturase, plastidial ω3-desaturase induced at low temperature, respectively.

FIG. 2. Schematic diagram of vector pOIL 122. Abbreviations: TER Agrtu-Nos, Agrobacterium tumefaciens nopaline synthase terminator; NPTII, neomycin phosphotransferase protein coding region; PRO CaMV35S-Ex2, Cauliflower Mosaic Virus 35S promoter with double enhancer region; Arath-DGAT1, Arabidopsis thaliana DGAT1 acyltransferase protein coding region; PRO Arath-Rubisco SSU, A. thaliana Rubisco small subunit promoter; Arath-FATA2, A. thaliana FATA2 thioesterase protein coding region; Arath-WRI, A. thaliana WRI1 transcription factor protein coding region; TER Glyma-Lectin, Glycine max lectin terminator; enTCUP2 promoter, Nicotiana tabacum cryptic constitutive promoter; attB1 and attB2, Gateway recombination sites; NB SDP1 fragment, Nicotiana benthamiana SDP1 region targeted for hpRNAi silencing; OCS terminator, A. tumefaciens octopine synthase terminator. Backbone features outside the T-DNA region are derived from pORE04 (Coutu et al., 2007).

FIG. 3. TAG levels (% leaf dry weight) in N. benthamiana leaf tissue, infiltrated with genes encoding different WRI1 polypeptides either with (right hand bars) or without (left hand bars) co-expression of DGAT1 (n=3). All samples were infiltrated with the P19 construct as well.

FIG. 4. Phylogenetic tree of LDAP polypeptides (Example 6).

FIG. 5. Schematic representation of the genetic construct pJP3506 including the T-DNA region between the left and right borders. TAG, triacylglycerol; FFA, free fatty acids; DAG, diacylglycerol; Sesin-Oleosin, Sesame indicum oleosin protein coding region.

FIG. 6. Triacylglycerol accumulation upon the expression of AtCaleosin 3 and SiOleosin L.

FIG. 7. Total fatty acid methyl ester (FAME) profiles (weight %) illustrating the effect of WRI1+DGAT1-mediated high oil background on MCFA production in Nicotiana benthamiana leaf (n=4). Highest MCFA production was observed after the addition of Arath-WRI1.

FIG. 8. TAG content in leaf samples of transformed tobacco plants at seed-setting stage of growth, transformed with the T-DNA from pOIL049, lines #23c and #32b. The controls (parent) samples were from plants transformed with the T-DNA from pJP3502. The upper line shows 18:2 percentage in the TAG and the lower line shows the 18:3 (ALA) percentage in the fatty acid content.

FIG. 9. Leaf total FAME profiles (weight %) elucidating the effect of WRI1 on MCFA accumulation (n=4). Addition of Arath-WRI1 greatly increased the production of the relevant fatty acid (C12:0, C14:0 or C16:0) relative to the previous addition of Cocnu-LPAAT alone.

FIG. 10. TFA levels (% weight), TAG levels, levels of MCFA (C16:0 and C14:0, % of total fatty acids) in TFA and MCFA in TAG (% of total fatty acid content in TAG) in plant cells after expression of combinations of three oil palm DGATs with FATB, LPAAT and WRI1. Numbers 1-10 are as listed in the text (Example 10).

FIG. 11. Phylogenetic relationship of glycerol-3-phosphate acyltransferase (GPAT) genes from various species including the Arabidopsis thaliana (AtGPAT9) and Cocos nucifera (CnGPAT9) genes used in this study. The plant GPAT9 cluster is shaded in grey. BrGPAT3=Brassica rapa glycerol-3-phosphate acyltransferase 3-like (Accession: XM_009105753); BnGPAT3=Brassica napus glycerol-3-phosphate acyltransferase 3-like (Accession: XM_013896062); CsGPAT3=Camelina sativa glycerol-3-phosphate acyltransferase 3 (Accession: XM_010458322); AtGPAT9=A. thaliana glycerol-3-phosphate acyltransferase 9 (Accession: NM_125455); ThGPAT3=Tarenaya hassleriana glycerol-3-phosphate acyltransferase 3-like (Accession: XM_010549847); RcGPAT3=Ricinus communis glycerol-3-phosphate acyltransferase 3 (Accession: NM_001323761); JcGPAT3=Jatropha curcas glycerol-3-phosphate acyltransferase 3 (Accession: NM_001308751); EgGPAT3=Elaeis guineensis glycerol-3-phosphate acyltransferase 3-like (Accession: XM_010913693); CnGPAT9=C. nucifera GPAT9 (Accession: KX235871); Mouse GPAT=Mus musculus 1-acylglycerol-3-phosphate O-acyltransferase 9 (Accession: NM_172715); LrGPAT=Lilium regale GPAT (Accession: JX524740); LpGPAT=Lilium pensylvanicum GPAT (Accession: JX524741); LlGPAT=Lilium longiflorum GPAT (Accession: JX524738); EgGPAT mRMA=E. guineensis mRNA for acylation enzyme (Accession: AJ272082); ChGPAT=Corylus heterophylla GPAT (Accession: JF428134); JcGPAT=J. curcas glycerol-3-phosphate acyltransferase, chloroplastic (Accession: NM_001305998); BnGPAT=B. napus glycerol-3-phosphate acyltransferase gene (Accession: KM243174); PsGPAT=Pisum sativum chloroplast mRNA for acyl-ACP:sn-glycerol-3-phosphate-acyltransferase (Accession: X59041); SlGPAT=Solanum lycopersicum glycerol-3-phosphate acyltransferase (Accession: NM_001306067); AtGPAT3=A. thaliana putative sn-glycerol-3-phosphate 2-O-acyltransferase (Accession: NM_116426); AtGPAT2=A. thaliana glycerol-3-phosphate sn-2-acyltransferase 2 (Accession: NM_100120); AtGPAT1=A. thaliana sn-glycerol-3-phosphate 2-O-acyltransferase (Accession: NM_100531); AtGPAT7=A. thaliana glycerol-3-phosphate acyltransferase 7 (Accession: NM_120691); AtGPAT5=A. thaliana glycerol-3-phosphate acyltransferase 5 (Accession: NM_111976); QSGPAT=Quercus suber glycerol-3-phosphate acyltransferase (Accession: JN819185); EgGPAT5=E. guineensis glycerol-3-phosphate acyltransferase 5 (Accession: XM_010923983); EgGPAT6=E. guineensis glycerol-3-phosphate 2-O-acyltransferase 6 (Accession: XM_010924793); AtGPAT6=A. thaliana bifunctional sn-glycerol-3-phosphate 2-O-acyltransferase/phosphatase (Accession: NM_129367); AtGPAT8=A. thaliana bifunctional sn-glycerol-3-phosphate 2-O-acyltransferase/phosphatase (Accession: NM_116264); AtGPAT4=A. thaliana glycerol-3-phosphate sn-2-acyltransferase (Accession: NM_100043); GhGPAT=Gossypium hirsutum probable glycerol-3-phosphate acyltransferase 3 (Accession: XM 016838669); EgGPAT4=E. guineensis glycerol-3-phosphate 2-O-acyltransferase 4-like (Accession: XM_010942191).

FIG. 12. Testing the effect of GPAT9 genes from Arabidopsis thaliana (AtGPAT9) and from Cocos nucifera (CnGPAT9) expression on TAG content, determined by transient Nicotiana benthamiana leaf expression (n=4).

FIG. 13. Fatty acid composition analysis of triacylglycerol (TAG), determined by the analysis of fatty acid methyl esters (FAME) via gas chromatography-flame ionisation detection (GC-FID) (n=3). Each thioesterase was infiltrated in combination with CnGPAT9 alone, CnGPAT9+CnLPAAT or CnGPAT9+CnLPAAT+EgDGAT1, with all treatments including expression of AtWRI1 (Arabidopsis thaliana WRINKLED1). CcTE=Cinnamomum camphora thioesterase; CnTE2=Cocos nucifera thioesterase; UcTE=Umbellularia californica thioesterase; CnGPAT9=C. nucifera glycerol-3-phosphate acyltransferase 9; CnLPAAT=C. nucifera lysophosphatidic acid acyltransferase; EgDGAT1=Elaeis guineensis diacylglycerol acyltransferase.

### KEY TO THE SEQUENCE LISTING

SEQ ID NO:1 Arabidopsis thaliana DGAT1 polypeptide (CAB44774.1)
SEQ ID NO:2 YFP tripeptide-conserved DGAT2 and/or MGAT1/2 sequence motif
SEQ ID NO:3 HPHG tetrapeptide-conserved DGAT2 and/or MGAT1/2 sequence motif
SEQ ID NO:4 EPHS tetrapeptide-conserved plant DGAT2 sequence motif
SEQ ID NO:5 RXGFX(K/R)XAXXXGXXX(L/V)VPXXXFG(E/Q)-long conserved sequence motif of DGAT2 which is part of the putative glycerol phospholipid domain
SEQ ID NO:6 FLXLXXXN-conserved sequence motif of mouse DGAT2 and MGAT1/2 which is a putative neutral lipid binding domain
SEQ ID NO:7 Conserved GPAT amino acid sequence GDL VICPEGTTCREP
SEQ ID NO:8 Conserved GPAT/phosphatase amino acid sequence (Motif I)
SEQ ID NO:9 Conserved GPAT/phosphatase amino acid sequence (Motif III)
SEQ ID NO:10 Sorbi-WRL1
SEQ ID NO:11 Lupan-WRL1
SEQ ID NO:12 Ricco-WRL1
SEQ ID NO:13 Lupin angustifolius WRI1 polypeptide
SEQ ID NO:14 WRI1 motif (R G V T/S R H R W T G R)
SEQ ID NO:15 WRI1 motif (F/Y E A H L W D K)
SEQ ID NO:16 WRI1 motif (D L A A L K Y W G)
SEQ ID NO:17 WRI1 motif (S X G F S/A R G X)
SEQ ID NO:18 WRI1 motif (H H H/Q N G R/K W E A R I G R/K V)
SEQ ID NO:19 WRI1 motif (Q E E A A A X Y D)
SEQ ID NO:20 pJP3502 TDNA (inserted into genome) sequence
SEQ ID NO:21 pJP3507 vector sequence
SEQ ID NO:22 Linker sequence
SEQ ID NO:23 Partial Nicotiana benthamiana CGI-58 sequence selected for hpRNAi silencing (pTV46)
SEQ ID NO:24 Partial N. tabacum AGPase sequence selected for hpRNAi silencing (pTV35)
SEQ ID NO:25 GXSXG lipase motif
SEQ ID NO:26 HX(4)D acyltransferase motif
SEQ ID NO:27 VX(3)HGF probable lipid binding motif
SEQ ID NO:28 Arabidopsis thaliana BBM polypeptide (NP_197245.2)
SEQ ID NO:29 Inducible Aspergillus niger alcA promoter
SEQ ID NO:30 AlcR inducer that activates the AlcA promotor in the presence of ethanol
SEQ ID NO:31 Arabidopsis thaliana LEC1; (AAC39488)
SEQ ID NO:32 Zea mays LEC1 (AAK95562)
SEQ ID NO:33 Arabidopsis thaliana LEC1-like (AAN15924)
SEQ ID NO:34 Arabidopsis thaliana FUS3 (AAC35247)
SEQ ID NO:35 Brassica napus FUS3
SEQ ID NO:36 Medicago truncatula FUS3
SEQ ID NO:37 Arabidopsis thaliana SDP1 cDNA sequence, Accession No. NM_120486, 3275 nt
SEQ ID NO:38 Sorghum bicolor SDP1 cDNA XM 002458486; 2724nt
SEQ ID NO:39 Nicotiana benthamiana SDP1 cDNA, Nbv5tr6404201SEQ ID NO:40 Nicotiana benthamiana SDP1 cDNA region targeted for hpRNAi silencing
SEQ ID NO:41 Promoter of Arabidopsis thaliana SDP 1 gene, 1.5 kb
SEQ ID NO:42 Nucleotide sequence of the complement of the pSSU-Oleosin gene in the T-DNA of pJP3502. In order (complementary sequences): Glycine max Lectin terminator 348nt, 3′ exon 255nt, UBQ10 intron 304nt, 5′ exon 213nt, SSU promoter 1751nt
SEQ ID NO:43 Arabidopsis thaliana FATA1
SEQ ID NO:44 Arabidopsis thaliana FATA2
SEQ ID NO:45 Arabidopsis thaliana FATB
SEQ ID NO:46 Arabidopsis thaliana WRI3
SEQ ID NO:47 Arabidopsis thaliana WRI4
SEQ ID NO:48 Avena sativa WRI1
SEQ ID NO:49 Sorghum bicolor WRI1
SEQ ID NO:50 Zea mays WRI1
SEQ ID NO:51 Triadica sebifera WRI1
SEQ ID NO:52 S. tuberosum Patatin B33 promoter sequence
SEQ ID NO:53 Z. mays SEE1 promoter region (1970nt from Accession number AJ494982)
SEQ ID NO:54 A. littoralis AlSAP promoter sequence, Accession No DQ885219
SEQ ID NO:55 A. rhizogenes ArRolC promoter sequence, Accession No. DQ160187
SEQ ID NO:56 Elaeis guineensis (oil palm) DGAT1
SEQ ID NO:57 G. max MYB73, Accession No. ABH02868
SEQ ID NO:58 A. thaliana bZIP53, Accession No. AAM14360
SEQ ID NO:59 A. thaliana AGL15, Accession No NP 196883
SEQ ID NO:60 A. thaliana MYB118, Accession No. AAS58517
SEQ ID NO:61 A. thaliana MYB115, Accession No. AAS10103
SEQ ID NO:62 A. thaliana TANMEI, Accession No. BAE44475
SEQ ID NO:63 A. thaliana WUS, Accession No. NP 565429
SEQ ID NO:64 B. napus GFR2al, Accession No. AFB74090
SEQ ID NO:65 B. napus GFR2a2, Accession No. AFB74089
SEQ ID NO:66 A. thaliana PHR 1, Accession No. AAN72198
SEQ ID NO:67 Sapium sebiferum LDAP-1 nucleotide sequence
SEQ ID NO:68 Sapium sebiferum LDAP-1 amino acid sequence
SEQ ID NO:69 Sapium sebiferum LDAP-2 nucleotide sequence
SEQ ID NO:70 Sapium sebiferum LDAP-2 amino acid sequence
SEQ ID NO:71 Sapium sebiferum LDAP-3 nucleotide sequence
SEQ ID NO:72 Sapium sebiferum LDAP-3 amino acid sequence
SEQ ID NO:73 S. bicolor SDP1 (accession number XM_002463620)
SEQ ID NO:74 T. aestivum SDP1 nucleotide sequence (Accession number AK334547)
SEQ ID NO:75 S. bicolor SDP1 hpRNAi fragment.
SEQ ID NO's 76 to 81 Oligonucleotide primer sequence
SEQ ID NO:82 Saccharum hybrid DIRIGENT (DIR16) promoter sequence
SEQ ID NO:83 Saccharum hybrid O-Methyl transferase (OMT) promoter sequence
SEQ ID NO:84 Sequence of the A1 promoter allele of the Saccharum hybrid R1MYB1 gene
SEQ ID NO:85 Saccharum hybrid Loading Stem Gene 5 (LSG5) promoter sequence
SEQ ID NO:86 Amino acid sequence of Sesamum indicum oleosinL polypeptide (Accession No. AF091840)
SEQ ID NO:87 Amino acid sequence of Cinnamomum camphora 14:0-ACP thioesterase (Accession No. Q39473.1)
SEQ ID NO:88 Amino acid sequence of Cocos nucifera acyl-ACP thioesterase FatB1 (Accession No. AEM72519.1)
SEQ ID NO:89 Amino acid sequence of Cocos nucifera acyl-ACP thioesterase FatB2 (Accession No. AEM72520.1)
SEQ ID NO:90 Amino acid sequence of Cocos nucifera acyl-ACP thioesterase FatB3 (Accession No. AEM72521.1)
SEQ ID NO:91 Amino acid sequence of Cuphea lanceolata acyl-(ACP) thioesterase type B (Accession No. CAB60830.1)
SEQ ID NO:92 Amino acid sequence of Cuphea viscosissima FatB1 (Accession No. AEM72522.1) SEQ ID NO:93 Amino acid sequence of and Umbellularia californica 12:0-ACP thioesterase (Accession No. Q41635.1)
SEQ ID NO:94 Amino acid sequence of C. nucifera LPAAT (Accession No. Q42670.1)
SEQ ID NO:95 Amino acid sequence of A. thaliana plastidial LPAAT1 (Accession No. AEE85783.1)
SEQ ID NO:96 Codon optimised nucleotide sequence of Elaeis guineensis DGAT1
SEQ ID NO:97 Amino acid sequence of Cocos nucifera GPAT9
SEQ ID NO:98 Amino acid sequence of Arabidopsis thaliana GPAT9
SEQ ID NO:99 Amino acid sequence of Elaeis guineensis GPAT9
SEQ ID NO:100 Amino acid sequence of Phoenix dactylifera GPAT9
SEQ ID NO:101 Amino acid sequence of Musa acuminata GPAT9
SEQ ID NO:102 Amino acid sequence of Ananas comosus GPAT9
SEQ ID NO:103 Amino acid sequence of Asparagus officinalis GPAT9
SEQ ID NO:104 Amino acid sequence of Oryza brachyantha GPAT9
SEQ ID NO:105 Amino acid sequence of Oryza sativa GPAT9
SEQ ID NO:106 Amino acid sequence of Nelumbo nucifera GPAT9
SEQ ID NO:107 Amino acid sequence of Vitis vinifera GPAT9
SEQ ID NO:108 Amino acid sequence of Nicotiana tomentosiformis GPAT9
SEQ ID NO:109 Amino acid sequence of Jatropha curcas GPAT9
SEQ ID NO:110 Amino acid sequence of Glycine max GPAT9
SEQ ID NO:111 Amino acid sequence of Sesamum indicum GPAT9
SEQ ID NO:112 Amino acid sequence of Brachypodium distachyon GPAT9
SEQ ID NO:113 Amino acid sequence of Setaria italica GPAT9
SEQ ID NO:114 Amino acid sequence of Cicer arietimum GPAT9
SEQ ID NO:115 Amino acid sequence of Zea mays GPAT9
SEQ ID NO:116 Amino acid sequence of Gossypium hirsutum GPAT9
SEQ ID NO:117 Amino acid sequence of Eucalyptus grandis GPAT9
SEQ ID NO:118 Amino acid sequence of Cucumis sativus GPAT9
SEQ ID NO:119 Amino acid sequence of Gossypium arboreum GPAT9
SEQ ID NO:120 Nucleotide sequence of Cocos nucifera GPAT9
SEQ ID NO:121 Nucleotide sequence of Arabidopsis thaliana GPAT9
SEQ ID NO:122 Nucleotide sequence of Elaeis guineensis GPAT9
SEQ ID NO:123 Nucleotide sequence of Phoenix dactylifera GPAT9
SEQ ID NO:124 Nucleotide sequence of Musa acuminata GPAT9
SEQ ID NO:125 Nucleotide sequence of Ananas comosus GPAT9
SEQ ID NO:126 Nucleotide sequence of Asparagus officinalis GPAT9
SEQ ID NO:127 Nucleotide sequence of Oryza brachyantha GPAT9
SEQ ID NO:128 Nucleotide sequence of Oryza sativa GPAT9
SEQ ID NO:129 Nucleotide sequence of Nelumbo nucifera GPAT9
SEQ ID NO:130 Nucleotide sequence of Vitis vinifera GPAT9
SEQ ID NO:131 Nucleotide sequence of Nicotiana tomentosiformis GPAT9
SEQ ID NO:132 Nucleotide sequence of Jatropha curcas GPAT9
SEQ ID NO:133 Nucleotide sequence of Glycine max GPAT9
SEQ ID NO:134 Nucleotide sequence of Sesamum indicum GPAT9
SEQ ID NO:135 Nucleotide sequence of Brachypodium distachyon GPAT9
SEQ ID NO:136 Nucleotide sequence of Setaria italica GPAT9
SEQ ID NO:137 Nucleotide sequence of Cicer arietinum GPAT9
SEQ ID NO:138 Nucleotide sequence of Zea mays GPAT9
SEQ ID NO:139 Nucleotide sequence of Gossypium hirsutum GPAT9
SEQ ID NO:140 Nucleotide sequence of Eucalyptus grandis GPAT9
SEQ ID NO:141 Nucleotide sequence of Cucumis sativus GPAT9
SEQ ID NO:142 Nucleotide sequence of Gossypium arboreum GPAT9
SEQ ID NO:143 Amino acid sequence of F. guineensis NF-YB1
SEQ ID NO:144 Amino acid sequence of E. guineensis ZFP1
SEQ ID NO:145 Amino acid sequence of A. thaliana NF-YB2
SEQ ID NO:146 Amino acid sequence of A. thaliana NF-YB3
SEQ ID NO:147 Amino acid sequence of A. thaliana ZFP2
SEQ ID NO:148 Amino acid sequence of E. guineensis ABI5
SEQ ID NO:149 Amino acid sequence of E. guineensis NF-YC2
SEQ ID NO:150 Amino acid sequence of E. guineensis NF-YA3
SEQ ID NO:151 Amino acid sequence of G. max DOF4
SEQ ID NO:152 Amino acid sequence of G. max ZF351

### DETAILED DESCRIPTION OF THE INVENTION

### General Techniques

Unless specifically defined otherwise, all technical and scientific terms used herein shall be taken to have the same meaning as commonly understood by one of ordinary skill in the art (e.g., in cell culture, molecular genetics, plant biology, cell biology, protein chemistry, lipid and fatty acid chemistry, animal nutrition, biofeul production, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

### Selected Definitions

The term “exogenous” in the context of a polynucleotide or polypeptide refers to the polynucleotide or polypeptide when present in a cell or a plant or part thereof which does not naturally comprise the polynucleotide or polypeptide. Such a cell is referred to herein as a “recombinant cell” or a “transgenic cell” and a plant comprising the cell as a “transgenic plant”. In an embodiment, the exogenous polynucleotide or polypeptide is from a different genus to the cell of the plant or part thereof comprising the exogenous polynucleotide or polypeptide. In another embodiment, the exogenous polynucleotide or polypeptide is from a different species. In one embodiment, the exogenous polynucleotide or polypeptide expressed in the plant cell is from a different species or genus. The exogenous polynucleotide or polypeptide may be non-naturally occurring, such as for example, a synthetic DNA molecule which has been produced by recombinant DNA methods. The DNA molecule may, preferably, include a protein coding region which has been codon-optimised for expression in the plant cell, thereby producing a polypeptide which has the same amino acid sequence as a naturally occurring polypeptide, even though the nucleotide sequence of the protein coding region is non-naturally occurring. The exogenous polynucleotide may encode, or the exogenous polypeptide may be, for example: a diacylglycerol acyltransferase (DGAT) such as a DGAT1 or a DGAT2, a Wrinkled 1 (WRI1) transcription factor, on OBC such as an Oleosin or preferably an LDAP, a fatty acid thioesterase such as a FATA or FATB polypeptide, or a silencing suppressor polypeptide. In an embodiment, a cell of the invention is a recombinant cell.

As used herein, the term “triacylglycerol (TAG) content” or variations thereof refers to the amount of TAG in the cell, plant or part thereof. TAG content can be calculated using techniques known in the art such as the sum of glycerol and fatty acyl moieties using a relation: % TAG by weight=100×((41×total mol FAME/3)+(total g FAME−(15×total mol FAME)))/g, where 41 and 15 are molecular weights of glycerol moiety and methyl group, respectively (where FAME is fatty acid methyl esters) (see Examples such as Example 1).

As used herein, the term “total fatty acid (TFA) content” or variations thereof refers to the total amount of fatty acids in the cell, plant or part thereof on a weight basis, as a percentage of the weight of the cell, plant or part thereof. Unless otherwise specified, the weight of the cell, plant or part thereof is the dry weight of the cell, plant or part thereof. TFA content is measured as described in Example 1 herein. The method involves conversion of the fatty acids in the sample to FAME and measurement of the amount of FAME by GC, using addition of a known amount of a distinctive fatty acid standard such as C17:0 as a quantitation standard in the GC. TFA therefore represents the weight of just the fatty acids, not the weight of the fatty acids and their linked moieties in the plant lipid.

As used herein, the“TAG/TFA Quotient” or “TTQ” parameter is calculated as the level of TAG (%) divided by the level of TFA (%), each as a percentage of the dry weight of the plant material. For example, a TAG level of 6% comprised in a TFA level of 10% yields a TTQ of 0.6. The TAG and TFA levels are measured as described herein. It is understood that, in this context, the TFA level refers to the weight of the total fatty acid content and the TAG level refers to the weight of TAG, including the glycerol moiety of TAG.

As used herein, the term “soluble protein content” or variations thereof refers to the amount of soluble protein in the plant or part thereof. Soluble protein content can be calculated using techniques known in the art. For instance, fresh tissue can be ground, chlorophyll and soluble sugars extracted by heating to 80° C. in 50-80% (v/v) ethanol in 2.5 mM HEPES buffer at pH 7.5, centrifugation, washing pellet in distilled water, resuspending the pellet 0.1 M NaOH and heating to 95° C. for 30 min, and then the Bradford assay (Bradford, 1976) is used determined soluble protein content. Alternatively, fresh tissue can be ground in buffer containing 100 mM Tris-HCl pH 8.0 and 10 mM MgCl₂.

As used herein, the term “nitrogen content” or variations thereof refers to the amount of nitrogen in the plant or part thereof. Nitrogen content can be calculated using techniques known in the art. For example, freeze-dried tissue can be analysed using a Europa 20-20 isotope ratio mass spectrometer with an ANCA preparation system, comprising a combustion and reduction tube operating at 1000° C. and 600° C., respectively, to determine nitrogen content.

As used herein, the term “carbon content” or variations thereof refers to the amount of carbon in the plant or part thereof. Carbon content can be calculated using techniques known in the art. For example, organic carbon levels can be determined using the method described by Shaw (1959), or as described in Example 1 of WO 2016/004473.

As used herein, the term “carbon:nitrogen ratio” or variations thereof refers to the relative amount of carbon in the cell, plant or part thereof when compared to the amount of nitrogen in the cell, plant or part thereof. Carbon and nitrogen contents can be calculated as described above and represented as a ratio.

As used herein, the term “photosynthetic gene expression” or variations thereof refers to one or more genes expressing proteins involved in photosynthetic pathways in the plant or part thereof. Examples of photosynthetic genes which may be upregulated in plants or parts thereof of the invention include, but are not limited to, one or more of the genes listed in Table 10 of WO 2016/004473.

As used herein, the term “photosynthetic capacity” or variations thereof refers to the ability of the plant or part thereof to photosynthesize (convert light energy to chemical energy). Photosynthetic capacity (Aₘₐₓ) is a measure of the maximum rate at which leaves are able to fix carbon during photosynthesis. It is typically measured as the amount of carbon dioxide that is fixed per metre squared per second, for example as μmol m⁻² sec⁻¹. Photosynthetic capacity can be calculated using techniques known in the art.

As used herein, the term “total dietary fibre (TDF) content” or variations thereof refers to the amount of fiber (including soluble and insoluble fibre) in the cell, plant or part thereof. As the skilled person would understand, dietary fiber includes non-starch polysaccharides such as arabinoxylans, cellulose, and many other plant components such as resistant starch, resistant dextrins, inulin, lignin, chitins, pectins, β-glucans, and oligosaccharides. TDF can be calculated using techniques known in the art. For example, using the Prosky method (Prosky et al. 1985), the McCleary method (McCleary et al., 2007) or the rapid integrated total dietary fiber method (McCleary et al., 2015).

As used herein, the term “energy content” or variations thereof refers to the amount of food energy in the plant or part thereof. More specifically, the amount of chemical energy that animals (including humans) derive from their food. Energy content can be calculated using techniques known in the art. For example, energy content can be determined based on heats of combustion in a bomb calorimeter and corrections that take into consideration the efficiency of digestion and absorption and the production of urea and other substances in the urine. As another example, energy content can be calculated as described in Example 1 of WO 2016/004473.

As used herein, the term “extracted lipid” refers to a composition extracted from a cell, plant or part thereof of the invention, such as a transgenic cell, plant or part thereof of the invention, which comprises at least 60% (w/w) lipid.

As used herein, the term “non-polar lipid” refers to fatty acids and derivatives thereof which are soluble in organic solvents but insoluble in water. The fatty acids may be free fatty acids and/or in an esterified form. Examples of esterified forms of non-polar lipid include, but are not limited to, triacylglycerol (TAG), diacylyglycerol (DAG), monoacylglycerol (MAG). Non-polar lipids also include sterols, sterol esters and wax esters. Non-polar lipids are also known as “neutral lipids”. Non-polar lipid is typically a liquid at room temperature. In an embodiment, at least 50%, more preferably at least 70%, more preferably at least 80%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99% of the fatty acids in non-polar lipid of the invention are present as TAG. The non-polar lipid may be further purified or treated, for example by hydrolysis with a strong base to release the free fatty acid, or by fractionation, distillation, or the like. Non-polar lipid may be present in or obtained from plant parts such as seed, leaves, tubers, beets or fruit. Non-polar lipid of the invention may form part of “seedoil” if it is obtained from seed.

The free and esterified sterol (for example, sitosterol, campesterol, stigmasterol, brassicasterol, 45-avenasterol, sitostanol, campestanol, and cholesterol) concentrations in the extracted lipid may be as described in Phillips et al. (2002). Sterols in plant oils are present as free alcohols, esters with fatty acids (esterified sterols), glycosides and acylated glycosides of sterols. Sterol concentrations in naturally occurring vegetable oils (seedoils) ranges up to a maximum of about 1100 mg/100 g. Hydrogenated palm oil has one of the lowest concentrations of naturally occurring vegetable oils at about 60 mg/100 g. The recovered or extracted seedoils of the invention preferably have between about 100 and about 1000 mg total sterol/100 g of oil. For use as food or feed, it is preferred that sterols are present primarily as free or esterified forms rather than glycosylated forms. In the seedoils of the present invention, preferably at least 50% of the sterols in the oils are present as esterified sterols, except for soybean seedoil which has about 25% of the sterols esterified. The canola seedoil and rapeseed oil of the invention preferably have between about 500 and about 800 mg total sterol/100 g, with sitosterol the main sterol and campesterol the next most abundant. The corn seedoil of the invention preferably has between about 600 and about 800 mg total sterol/100 g, with sitosterol the main sterol. The soybean seedoil of the invention preferably has between about 150 and about 350 mg total sterol/100 g, with sitosterol the main sterol and stigmasterol the next most abundant, and with more free sterol than esterified sterol. The cottonseed oil of the invention preferably has between about 200 and about 350 mg total sterol/100 g, with sitosterol the main sterol. The coconut oil and palm oil of the invention preferably have between about 50 and about 100 mg total sterol/100 g, with sitosterol the main sterol. The safflower seedoil of the invention preferably has between about 150 and about 250 mg total sterol/100 g, with sitosterol the main sterol. The peanut seedoil of the invention preferably has between about 100 and about 200 mg total sterol/100 g, with sitosterol the main sterol. The sesame seedoil of the invention preferably has between about 400 and about 600 mg total sterol/100 g, with sitosterol the main sterol. The sunflower seedoil of the invention preferably has between about 200 and 400 mg total sterol/100 g, with sitosterol the main sterol. Oils obtained from vegetative plant parts according to the invention preferably have less than 200 mg total sterol/100 g, more preferably less than 100 mg total sterol/100 g, and most preferably less than 50 mg total sterols/100 g, with the majority of the sterols being free sterols. In an embodiment, the lipid or oil is from a vegetative plant part which comprises one or more or all of sitosterol, campesterol, stigmasterol and cholesterol. In an embodiment, the lipid or oil is from a vegetative plant part and has more galactosylglycerides than phosphoglycerides. In an embodiment, the lipid or oil is from a seed and has more phosphoglycerides than galactosylglycerides. Further guidance regarding sterols and other lipids components of plant cells can be found in Gunstone et al. (2007) The Lipid Handbook, Third Edition, CRC Press.

As used herein, the term “vegetative oil” refers to a composition obtained from vegetative parts of a plant which comprises at least 60% (w/w) lipid, or obtainable from the vegetative parts if the oil is still present in the vegetative part. That is, vegetative oil of the invention includes oil which is present in the vegetative plant part, as well as oil which has been extracted from the vegetative part (extracted oil). The vegetative oil is preferably extracted vegetative oil. Vegetative oil is typically a liquid at room temperature. The fatty acids are typically in an esterified form such as for example, TAG, DAG, acyl-CoA, galactolipid or phospholipid. The fatty acids may be free fatty acids and/or in an esterified form. In an embodiment, at least 50%, more preferably at least 70%, more preferably at least 80%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99% of the fatty acids in vegetative oil of the invention can be found as TAG. In an embodiment, vegetative oil of the invention is “substantially purified” or “purified” oil that has been separated from one or more other lipids, nucleic acids, polypeptides, or other contaminating molecules with which it is associated in the vegetative plant part or in a crude extract. It is preferred that the substantially purified vegetative oil is at least 60% free, more preferably at least 75% free, and more preferably, at least 90% free from other components with which it is associated in the vegetative plant part or extract. Vegetative oil of the invention may further comprise non-fatty acid molecules such as, but not limited to, sterols. In an embodiment, the vegetative oil is canola oil (Brassica sp. such as Brassica carinata, Brassica juncea, Brassica napobrassica, Brassica napus) mustard oil (Brassica juncea), other Brassica oil (e.g., Brassica napobrassica, Brassica camelina), sunflower oil (Helianthus sp. such as Helianthus annuus), linseed oil (Linum usitatissimum), soybean oil (Glycine max), safflower oil (Carthamus tinctorius), corn oil (Zea mays), tobacco oil (Nicotiana sp. such as Nicotiana tabacum or Nicotiana benthamiana), peanut oil (Arachis hypogaea), palm oil (Elaeis guineensis), cotton oil (Gossypium hirsutum), coconut oil (Cocos nucifera), avocado oil (Persea americana), olive oil (Olea europaea), cashew oil (Anacardium occidentale), macadamia oil (Macadamia intergrifolia), almond oil (Prunus amygdalus), oat oil (Avena sativa), rice oil (Oryza sp. such as Oryza sativa and Oryza glaberrima), Arabidopsis oil (Arabidopsis thaliana), Aracinis hypogaea (peanut), Beta vulgaris oil (sugar beet), Camelina sativa oil (false flax), Crambe abyssinica oil (Abyssinian kale), Cucumis melo oil (melon), Hordeum vulgare oil (barley), Jatropha curcas oil (physic nut), Joannesia princeps oil (arara nut-tree), Licania rigida oil (oiticica), Lupinus angustifolius oil (lupin), Miscanthus sp. oil such as Miscanthus x giganteus oil and Miscanthus sinensis oil, Panicum virgatum (switchgrass) oil, Pongamia pinnata oil (Indian beech), Populus trichocarpa oil, Ricinus communis oil (castor), Saccharum sp. oil (sugarcane), Sesamum indicum oil (sesame), Solanum tuberosum oil (potato), Sorghum sp. oil such as Sorghum bicolor oil, Sorghum vulgare oil, Theobroma grandiforum oil (cupuassu), Trifolium sp. oil, and Triticum sp. oil (wheat) such as Triticum aestivum. oil Vegetative oil may be extracted from vegetative plant parts by any method known in the art, such as for extracting seedoils. This typically involves extraction with nonpolar solvents such as diethyl ether, petroleum ether, chloroform/methanol or butanol mixtures, generally associated with first crushing of the seeds.

Lipids associated with the starch or other polysaccharides may be extracted with water-saturated butanol. The seedoil may be “de-gummed” by methods known in the art to remove polar lipids such as phospholipids or treated in other ways to remove contaminants or improve purity, stability, or colour. The TAGs and other esters in the vegetative oil may be hydrolysed to release free fatty acids, or the oil hydrogenated, treated chemically, or enzymatically as known in the art. As used herein, the term “seedoil” has an analogous meaning except that it refers to a lipid composition obtained from seeds of plants of the invention.

As used herein, the term “fatty acid” refers to a carboxylic acid with an aliphatic tail of at least 6 carbon atoms in length, either saturated or unsaturated. Preferred fatty acids have a carbon-carbon bonded chain of at least 12 carbons in length, more preferably fatty acids having have a carbon-carbon bonded chain of 12 and/or 14 carbons in length. Most naturally occurring fatty acids have an even number of carbon atoms because their biosynthesis involves acetate which has two carbon atoms. The fatty acids may be in a free state (non-esterified) or in an esterified form such as part of a TAG, DAG, MAG, acyl-CoA (thio-ester) bound, acyl-ACP bound, or other covalently bound form. When covalently bound in an esterified form, the fatty acid is referred to herein as an “acyl” group. The fatty acid may be esterified as a phospholipid such as a phosphatidylcholine (PC), phosphatidylethanolamine (PE), phosphatidylserine (PS), phosphatidylglycerol (PG), phosphatidylinositol (PI), or diphosphatidylglycerol. Saturated fatty acids do not contain any double bonds or other functional groups along the chain. The term “saturated” refers to hydrogen, in that all carbons (apart from the carboxylic acid [—COOH] group) contain as many hydrogens as possible. In other words, the omega (w) end contains 3 hydrogens (CH₃—) and each carbon within the chain contains 2 hydrogens (—CH₂—). Unsaturated fatty acids are of similar form to saturated fatty acids, except that one or more alkene functional groups exist along the chain, with each alkene substituting a singly-bonded “—CH₂—CH₂—” part of the chain with a doubly-bonded “—CH═CH—” portion (that is, a carbon double bonded to another carbon). The two next carbon atoms in the chain that are bound to either side of the double bond can occur in a cis or trans configuration.

As used herein, a fatty acid with a “medium chain length”, also referred to as “MCFA”, comprises an acyl chain of 6 to 14 carbons. The acyl chain may be modified (for example it may comprise one or more double bonds, a hydroxyl group, an expoxy group, etc) or preferably is a saturated MCFA. This terms at least includes one or more or all of caproic acid (C6:0), caprylic acid (C8:0), capric acid (C10:0), lauric acid (C12:0), and myristic acid (C14:0). In an embodiment, the medium chain length fatty acids are lauric acid and/or myristic acid, or capric, lauric and myristic.

As used herein, “new medium chain fatty acids” or “new medium chain fatty acid content” or the like refers to the difference between the total MCFA content of the extracted lipid, oil, recombinant cell, plant or plant part, or seed, of the invention as the context determines, expressed as a percentage of the total fatty acid content, and the total MCFA content of a corresponding wild-type extracted lipid, oil, recombinant cell, plant or plant part, or seed, obtained from a wild-type plant. That is, the new MCFA refers to the increased MCFA of the product of the invention relative to the corresponding wild-type product. These new medium chain fatty acids are the fatty acids that are produced in the cells, plants and plant parts, or seeds, of the invention by the expression of the genetic constructs (exogenous polynucleotides) introduced into the cells, and include (if present) lauric acid and/or myristic acid. Exemplary total medium chain fatty acid contents and new medium chain fatty acid contents are determined by conversion of fatty acids in a sample to FAME and analysis by GC, as described in Example 1.

As used herein, “new medium chain fatty acids in the total fatty acid content of the TAG of the extracted lipid” or the like refers to the difference of the total MCFA content esterified in the form of triacylglycerols in the extracted lipid, oil, recombinant cell, plant or plant part, or seed, as the context determines, expressed as a percentage of the total fatty acid content esterified in TAG, and the total MCFA content esterified in the form of triacylglycerols in a corresponding wild-type extracted lipid, oil, recombinant cell, plant or plant part, or seed, obtained from a wild-type plant.

As used herein, the terms “monounsaturated fatty acid” or “MUFA” refer to a fatty acid which comprises at least 12 carbon atoms in its carbon chain and only one alkene group (carbon-carbon double bond), which may be in an esterified or non-esterified (free) form. As used herein, the terms “polyunsaturated fatty acid” or “PUFA” refer to a fatty acid which comprises at least 12 carbon atoms in its carbon chain and at least two alkene groups (carbon-carbon double bonds), which may be in an esterified or non-esterified form.

“Monoacylglyceride” or “MAG” is glyceride in which the glycerol is esterified with one fatty acid. As used herein, MAG comprises a hydroxyl group at an sn-1/3 (also referred to herein as sn-1 MAG or 1-MAG or 1/3-MAG) or sn-2 position (also referred to herein as 2-MAG), and therefore MAG does not include phosphorylated molecules such as PA or PC. MAG is thus a component of neutral lipids in a plant or part thereof.

“Diacylglyceride” or “DAG” is glyceride in which the glycerol is esterified with two fatty acids which may be the same or, preferably, different. As used herein, DAG comprises a hydroxyl group at a sn-1,3 or sn-2 position, and therefore DAG does not include phosphorylated molecules such as PA or PC. DAG is thus a component of neutral lipids in a plant or part thereof. In the Kennedy pathway of DAG synthesis (FIG. 1), the precursor sn-glycerol-3-phosphate (G3P) is esterified to two acyl groups, each coming from a fatty acid coenzyme A ester, in a first reaction catalysed by a glycerol-3-phosphate acyltransferase (GPAT) at position sn-1 to form LysoPA, followed by a second acylation at position sn-2 catalysed by a lysophosphatidic acid acyltransferase (LPAAT) to form phosphatidic acid (PA). This intermediate is then de-phosphorylated by PAP to form DAG. DAG may also be formed from TAG by removal of an acyl group by a lipase, or from PC essentially by removal of a choline headgroup by any of the enzymes PDCT, PLC or PLD (FIG. 1).

“Triacylglyceride” or “TAG” is a glyceride in which the glycerol is esterified with three fatty acids which may be the same (e.g. as in tri-olein) or, more commonly, different. In the Kennedy pathway of TAG synthesis, DAG is formed as described above, and then a third acyl group is esterified to the glycerol backbone by the activity of DGAT. Alternative pathways for formation of TAG include one catalysed by the enzyme PDAT (FIG. 1) and the MGAT pathway described herein.

As used herein, the term “wild-type” or variations thereof refers to a cell, plant or part thereof such as a cell, vegetative plant part, seed, tuber or beet, that has not been genetically modified, such as cells, plants or parts thereof that do not comprise the one or more exogenous polynucleotides, according to this invention.

The term “corresponding” refers to a cell, plant or part thereof such as a cell, vegetative plant part, seed, tuber or beet, that has the same or similar genetic background as a cell, plant or part thereof such as a vegetative plant part, seed, tuber or beet of the invention but which has not been modified as described herein (for example, a vegetative plant part or seed which lacks the defined exogenous polynucleotide(s)). In a preferred embodiment, the corresponding plant or part thereof such as a vegetative plant part is at the same developmental stage as the plant or part thereof such as a vegetative plant part of the invention. For example, if the plant is a flowering plant, then preferably the corresponding plant is also flowering. A corresponding cell, plant or part thereof such as a vegetative plant part, can be used as a control to compare levels of nucleic acid or protein expression, or the extent and nature of trait modification, for example MCFA and/or TAG content, with the cell, plant or part thereof such as a vegetative plant part of the invention which is modified as described herein. A person skilled in the art is readily able to determine an appropriate “corresponding” cell, plant or part thereof such as a vegetative plant part for such a comparison.

As used herein, “compared with” or “relative to” refers to comparing levels of, for example, MCFA or triacylglycerol (TAG) content, one or more or all of soluble protein content, nitrogen content, carbon:nitrogen ratio, photosynthetic gene expression, photosynthetic capacity, total dietary fibre (TDF) content, carbon content, and energy content, or non-polar lipid content or composition, total non-polar lipid content, total fatty acid content or other parameter of the cell, plant or part thereof comprising the one or more exogenous polynucleotides, genetic modifications or exogenous polypeptides with a cell, plant or part thereof such as a vegetative plant part lacking the one or more exogenous polynucelotides, genetic modifications or polypeptides.

As used herein, “synergism”, “synergistic”, “acting synergistically” and related terms are each a comparative term that means that the effect of a combination of elements present in a plant or part thereof of the invention, for example a combination of elements A and B, is greater than the sum of the effects of the elements separately in corresponding plants or parts thereof, for example the sum of the effect of A and the effect of B. Where more than two elements are present in the plant or part thereof, for example elements A, B and C, it means that the effect of the combination of all of the elements is greater than the sum of the effects of the individual effects of the elements. In a preferred embodiment, it means that the effect of the combination of elements A, B and C is greater than the sum of the effect of elements A and B combined and the effect of element C. In such a case, it can be said that element C acts synergistically with elements A and B. As would be understood, the effects are measured in corresponding cells, plants or parts thereof, for example grown under the same conditions and at the same stage of biological development.

As used herein, “germinate at a rate substantially the same as for a corresponding wild-type plant” or similar phrases refers to seed of a plant of the invention being relatively able to germinate when compared to seed of a wild-type plant lacking the defined exogenous polynucleotide(s) and genetic modifications. Germination may be measured in vitro on tissue culture medium or in soil as occurs in the field. In one embodiment, the number of seeds which germinate, for instance when grown under optimal greenhouse conditions for the plant species, is at least 75%, more preferably at least 90%, when compared to corresponding wild-type seed. In another embodiment, the seeds which germinate, for instance when grown under optimal glasshouse conditions for the plant species, produce seedlings which grow at a rate which, on average, is at least 75%, more preferably at least 90%, when compared to corresponding wild-type plants. This is referred to as “seedling vigour”. In an embodiment, the rate of initial root growth and shoot growth of seedlings of the invention is essentially the same compared to a corresponding wild-type seedling grown under the same conditions. In an embodiment, the leaf biomass (dry weight) of the plants of the invention is at least 80%, preferably at least 90%, of the leaf biomass relative to a corresponding wild-type plant grown under the same conditions, preferably in the field. In an embodiment, the height of the plants of the invention is at least 70%, preferably at least 80%, more preferably at least 90%, of the plant height relative to a corresponding wild-type plant grown under the same conditions, preferably in the field and preferably at maturity.

As used herein, the term “an exogenous polynucleotide which down-regulates the production and/or activity of an endogenous polypeptide” or variations thereof, refers to a polynucleotide that encodes an RNA molecule, herein termed a “silencing RNA molecule” or variations thereof (for example, encoding an amiRNA or hpRNAi), that down-regulates the production and/or activity, or itself down-regulates the production and/or activity (for example, is an amiRNA or hpRNA which can be delivered directly to, for example, the plant or part thereof) of an endogenous polypeptide. This includes where the initial RNA transcript produced by expression of the exogenous polynucleotide is processed in the cell to form the actual silencing RNA molecule. The endogenous polypeptides whose production or activity are downregulated include, for example, SDP1 TAG lipase, plastidial GPAT, plastidial LPAAT, TGD polypeptide such as TGD5, TST such as TST1 or TST2, AGPase, PDCT, CPT or Δ12 fatty acid desturase (FAD2), or a combination of two or more thereof. Typically, the RNA molecule decreases the expression of an endogenous gene encoding the polypeptide. The extent of down-regulation is typically less than 100%, for example the production or activity is reduced by between 25% and 95% relative to the wild-type. The optimal level of remaining production or activity can be routinely determined.

As used herein, the term “on a weight basis” refers to the weight of a substance (for example, TAG, DAG, fatty acid, protein, nitrogen, carbon) as a percentage of the weight of the composition comprising the substance (for example, seed, leaf dry weight). For example, if a transgenic seed has 25 μg total fatty acid per 120 μg seed weight; the percentage of total fatty acid on a weight basis is 20.8%.

As used herein, the term “on a relative basis” refers to a parameter such as the amount of a substance in a composition comprising the substance in comparison with the parameter for a corresponding composition, as a percentage. For example, a reduction from 3 units to 2 units is a reduction of 33% on a relative basis.

As used herein, “plastids” are organelles in plants, including algae, which are the site of manufacture of carbon-based compounds from photosynthesis including sugars, starch and fatty acids. Plastids include chloroplasts which contain chlorophyll and carry out photosynthesis, etioplasts which are the predecessors of chloroplasts, as well as specialised plastids such as chromoplasts which are coloured plastids for synthesis and storage of pigments, gerontoplasts which control the dismantling of the photosynthetic apparatus during senescence, amyloplasts for starch synthesis and storage, elaioplasts for storage of lipids, and proteinoplasts for storing and modifying proteins.

As used herein, the term “biofuel” refers to any type of fuel, typically as used to power machinery such as automobiles, planes, boats, trucks or petroleum powered motors, whose energy is derived from biological carbon fixation. Biofuels include fuels derived from biomass conversion, as well as solid biomass, liquid fuels and biogases. Examples of biofuels include bioalcohols, biodiesel, synthetic diesel, vegetable oil, bioethers, biogas, syngas, solid biofuels, algae-derived fuel, biohydrogen, biomethanol, 2,5-Dimethylfuran (DMF), biodimethyl ether (bioDME), Fischer-Tropsch diesel, biohydrogen diesel, mixed alcohols and wood diesel.

As used herein, the term “bioalcohol” refers to biologically produced alcohols, for example, ethanol, propanol and butanol. Bioalcohols are produced by the action of microorganisms and/or enzymes through the fermentation of sugars, hemicellulose or cellulose.

As used herein, the term “biodiesel” refers to a composition comprising fatty acid methyl- or ethyl-esters derived from lipids by transesterification, the lipids being from living cells not fossil fuels.

As used herein, the term “synthetic diesel” refers to a form of diesel fuel which is derived from renewable feedstock rather than the fossil feedstock used in most diesel fuels.

As used herein, the term “vegetable oil” includes a pure plant oil (or straight vegetable oil) or a waste vegetable oil (by product of other industries), including oil produced in either a vegetative plant part or in seed. Vegetable oil includes vegetative oil and seedoil, as defined herein.

As used herein, the term “biogas” refers to methane or a flammable mixture of methane and other gases produced by anaerobic digestion of organic material by anaerobes.

As used herein, the term “syngas” refers to a gas mixture that contains varying amounts of carbon monoxide and hydrogen and possibly other hydrocarbons, produced by partial combustion of biomass. Syngas may be converted into methanol in the presence of catalyst (usually copper-based), with subsequent methanol dehydration in the presence of a different catalyst (for example, silica-alumina).

As used herein, the term “biochar” refers to charcoal made from biomass, for example, by pyrolysis of the biomass.

As used herein, the term “feedstock” refers to a material, for example, biomass or a conversion product thereof (for example, syngas) when used to produce a product, for example, a biofuel such as biodiesel or a synthetic diesel.

As used herein, the term “industrial product” refers to a hydrocarbon product which is predominantly made of carbon and hydrogen such as, for example, fatty acid methyl- and/or ethyl-esters or alkanes such as methane, mixtures of longer chain alkanes which are typically liquids at ambient temperatures, a biofuel, carbon monoxide and/or hydrogen, or a bioalcohol such as ethanol, propanol, or butanol, or biochar. The term “industrial product” is intended to include intermediary products that can be converted to other industrial products, for example, syngas is itself considered to be an industrial product which can be used to synthesize a hydrocarbon product which is also considered to be an industrial product. The term industrial product as used herein includes both pure forms of the above compounds, or more commonly a mixture of various compounds and components, for example the hydrocarbon product may contain a range of carbon chain lengths, as well understood in the art.

As used herein, “progeny” means the immediate and all subsequent generations of offspring produced from a parent, for example a second, third or later generation offspring.

As used herein, the term “ancestor” refers to any earlier generation of the plant comprising the first and second exogenous polynucleotides. The ancestor may be the parent plant, grandparent plant, great grandparent plant and so on.

As used herein, the term “selecting a plant” means actively selecting the plant on the basis that it has the desired phenotype, such as increased MCFA when compared to the corresponding wild-type plant.

As used herein, phrases such as “comprise a TFA content of about 5% (w/w dry weight)”, or “comprise a total TAG content of about 6% (w/w dry weight)”, or similarly structured phrases, mean that more than the defined level may be present. For instance, the phrase “comprise a TFA content of about 5% (w/w dry weight)” can be used interchangeably with “comprises at least about 5% TFA (w/w dry weight)”. Extending this example further, a vegetative plant part which comprise a TFA content of about 5% (w/w dry weight) may have a 6%, or 7.5% or higher TFA content.

As used herein, unless the context indicates otherwise, the term “increased content” when used in reference to a polypeptide, or similar phrases including reference to specific polypeptide, refers to either an exogenous polypeptide or an endogenous polypeptide. For example, a vegetative plant part of the invention may comprise an increased content of a WRI1 polypeptide, am increased GPAT9 content, an increased LPAAT content, an increased content of a DGAT polypeptide, and a decreased content of a SDP1 polypeptide, each relative to a corresponding wild-type vegetative plant part, wherein each of the WRI1 and DGAT polypeptides is independently either an exogenous polypeptide or an endogenous polypeptide. As another example, a vegetative plant part of the invention may comprise an increased content of a WRI1 polypeptide, an increased content of a DGAT polypeptide, and an increased content of a LEC2 polypeptide, each relative to a corresponding wild-type vegetative plant part, wherein each of the WRI1, DGAT and LEC2 polypeptides is independently either an exogenous polypeptide or an endogenous polypeptide. As a further example, a vegetative plant part of the invention may comprise an increased content of a PDAT or DGAT polypeptide, a decreased content of a TGD polypeptide, and a decreased content of a SDP1 polypeptide, each relative to a corresponding wild-type vegetative plant part wherein the PDAT or DGAT is either an exogenous polypeptide or an endogenous polypeptide, and so on. An exogenous polypeptide may be the result of expression of a transgene encoding the polypeptide in the cell or plant or part thereof of the invention. The endogenous polypeptide may be the result of increased expression of an endogenous gene, such as inducing overexpression and/or providing increased levels of a transcription factor(s) for the gene.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “and/or”, e.g., “X and/or Y” shall be understood to mean either “X and Y” or “X or Y” and shall be taken to provide explicit support for both meanings or for either meaning. As used herein, the term about, unless stated to the contrary, refers to +/−10%, more preferably +/−5%, more preferably +/−2%, more preferably +/−1%, even more preferably +/−0.5%, of the designated value.

Production of Plants with Modified Traits

The present invention is based on the finding that plant traits, such as MFCA content and TAG content, in plants or parts thereof can be increased by a combination of two or more modifications selected from those designated herein as: (A). Push, (B). Pull, (C). Protect, (D). Package, (E). Plastidial Export, (F). Plastidial Import and (G). Prokaryotic Pathway.

Plants or parts thereof such as a vegetative plant parts of the invention therefore have a number of combinations of exogenous polynucleotides and/or genetic modifications each of which provide for one of the modifications. These exogenous polynucleotides and/or genetic modifications include:
(A) an exogenous polynucleotide which encodes a transcription factor polypeptide that increases the expression of one or more glycolytic and/or fatty acid biosynthetic genes in the plant or part thereof such as a vegetative plant part, providing the “Push” modification,
(B) an exogenous polynucleotide which encodes a polypeptide involved in the biosynthesis of one or more non-polar lipids in the plant or part thereof such as a vegetative plant part, providing the “Pull” modification,
(C) a genetic modification which down-regulates endogenous production and/or activity of a polypeptide involved in the catabolism of triacylglycerols (TAG) in the plant or part thereof such as a vegetative plant part when compared to a corresponding plant or part thereof such as a vegetative plant part lacking the genetic modification, providing the “Protect” modification,
(D) an exogenous polynucleotide which encodes an oil body coating (OBC) polypeptide such as a lipid droplet associated polypeptide (LDAP), providing the “Package” modification,
(E) an exogenous polynucleotide which encodes a polypeptide which increases the export of fatty acids out of plastids of the plant or part thereof such as a vegetative plant part, when compared to a corresponding plant or part thereof such as a vegetative plant part lacking the exogenous polynucleotide, providing the “Plastidial Export” modification,
(F) a genetic modification which down-regulates endogenous production and/or activity of a polypeptide involved in importing fatty acids into plastids of the plant or part thereof such as a vegetative plant part when compared to a corresponding plant or part thereof such as a vegetative plant part lacking the genetic modification, providing the “Plastidial Import” modification, and
G) a genetic modification which down-regulates endogenous production and/or activity of a polypeptide involved in diacylglycerol (DAG) production in the plastid of the plant or part thereof such as a vegetative plant part when compared to a corresponding plant or part thereof such as a vegetative plant part lacking the genetic modification, providing the “prokaryotic Pathway” modification.

Preferred combinations (also referred to herein as sets) of exogenous polynucleotides and/or genetic modifications of the invention are;
1) A, B and optionally one of C, D, E, F or G;
2) A, C and optionally one of D, E, F or G;
3) A, D and optionally one of E, F or G;
4) A, E and optionally F or G;
5) A, F and optionally G;
6) A and G;
7) A, B, C and optionally one of D, E, F or G;
8) A, B, D and optionally one of E, F or G;
9) A, B, E and optionally F or G;
10) A, B, F and optionally G;
11) A, B, C, D and optionally one of E, F or G;
12) A, B, C, E and optionally F or G;
13) A, B, C, F and optionally G;
14) A, B, D, E and optionally F or G;
15) A, B, D, F and optionally G;
16) A, B, E, F and optionally G;
17) A, C, D and optionally one of E, F or G;
18) A, C, E and optionally F or G;
19) A, C, F and optionally G;
20) A, C, D, E and optionally F or G;
21) A, C, D, F and optionally G;
22) A, C, E, F and optionally a fifth modification G;
23) A, D, E and optionally F or G;
24) A, D, F and optionally G;
25) A, D, E, F and optionally G;
26) A, E, F and optionally G;
27) Six of A, B, C, D, E, F and G omitting one of A, B, C, D, E, F or G, and
28) Any one of 1-26 above where there are two or more exogenous polynucleotides encoding two or more different transcription factor polypeptides that increases the expression of one or more glycolytic and/or fatty acid biosynthetic genes in the plant or part thereof, for example one exogenous polynucleotide encoding WRI1 and another exogenous polynucleotide encoding LEC2.

In each of the above preferred combinations there may be at least two different exogenous polynucleotides which encode at least two different transcription factor polypeptides that increases the expression of one or more glycolytic and/or fatty acid biosynthetic genes in the plant or part thereof such as a vegetative plant part.

These modifications are described more fully as follows:

A. The “Push” modification is characterised by an increased synthesis of total fatty acids in the plastids of the plant or part thereof. In an embodiment, this occurs by the increased expression and/or activity of a transcription factor which regulates fatty acid synthesis in the plastids. In one embodiment, this can be achieved by expressing in a transgenic plant or part thereof an exogenous polynucleotide which encodes a transcription factor polypeptide that increases the expression of one or more glycolytic and/or fatty acid biosynthetic genes in the plant or part thereof. In an embodiment, the increased fatty acid synthesis is not caused by the provision to the plant or part thereof of an altered ACCase whose activity is less inhibited by fatty acids, relative to the endogenous ACCase in the plant or part thereof. In an embodiment, the plant or part thereof comprises an exogenous polynucleotide which encodes the transcription factor, preferably under the control of a promoter other than a constitutive promoter. The transcription factor may be selected from the group consisting of WRI1, LEC1, LEC1-like, LEC2, BBM, FUS3, ABI3, ABI4, ABI5, Dof4, Dof11 or the group consisting of MYB73, bZIP53, AGL15, MYB115, MYB118, TANMEI, WUS, GFR2al, GFR2a2 and PHR1, and is preferably WRI1, LEC1 or LEC2, or WRI1 alone. In a further embodiment, the increased synthesis of total fatty acids is relative to a corresponding wild-type plant or part thereof. In an embodiment, there are two or more exogenous polynucleotides encoding two or more different transcription factor polypeptides. The “Push” modification may also be achieved by increased expression of polypeptides which modulate activity of WRI1, such as MED15 or 14-3-3 polypeptides.

B. The “Pull” modification is characterised by increased expression and/or activity in the plant or part thereof of a fatty acyl acyltransferase which catalyses the synthesis of TAG, DAG or MAG in the plant or part thereof, such as a DGAT, PDAT, LPAAT, GPAT or MGAT, preferably a DGAT or a PDAT. In one embodiment, this can be achieved by expressing in a transgenic plant or part thereof an exogenous polynucleotide which encodes a polypeptide involved in the biosynthesis of one or more non-polar lipids. In an embodiment, the acyltransferase is a membrane-bound acyltransferase that uses an acyl-CoA substrate as the acyl donor in the case of DGAT, LPAAT, GPAT or MGAT, or an acyl group from PC as the acyl donor in the case of PDAT. The Pull modification can be relative to a corresponding wild-type plant or part thereof or, preferably, relative to a corresponding plant or part thereof which has the Push modification. In an embodiment, the plant or part thereof comprises an exogenous polynucleotide which encodes the fatty acyl acyltransferase. The “Pull” modification can also be achieved by increased expression of a PDCT, CPT or phospholipase C or D polypeptide which increases the production of DAG from PC.

In a preferred embodiment, the cell comprises an exogenous polynucleotide(s) encoding one or more or all of a GPAT, LPAAT and/or DGAT which have a preference for utilising medium chain fatty acid substrates, particularly for lauric acid and/or myristic acid. Such GPAT, LPAAT and/or DGAT having a preference for utilising medium chain fatty acid substrates include those described herein, as well as those which can be isolated from plants which naturally produce high levels of medium chain fatty acids, such as but not limited to, Elaeis guineensis, Cocus nucifera, Attalea dubia, Orbignya phalerata, Astrocaryum murumuru, Bactris gasipaes, Pycnanthus angolensis, Cuphea wrightii, Attalea colenda, Laurus nobilis, Umbellularia californica, Qualea grandiflora and Actinodaphne hookeri. The skilled person would appreciate that the sequences provided herein which readily be used to screen sequence databases to identify orthologous genes and proteins from the above species.

C. The “Protect” modification is characterised by a reduction in the catabolism of triacylglycerols (TAG) in the plant or part thereof. In an embodiment, this can be achieved through a genetic modification in the plant or part thereof which down-regulates endogenous production and/or activity of a polypeptide involved in the catabolism of triacylglycerols (TAG) in the plant or part thereof when compared to a corresponding plant or part thereof lacking the genetic modification. In an embodiment, the plant or part thereof has a reduced expression and/or activity of an endogenous TAG lipase in the plant or part thereof, preferably an SDP1 lipase, a Cgi58 polypeptide, an acyl-CoA oxidase such as the ACX1 or ACX2, or a polypeptide involved in β-oxidation of fatty acids in the plant or part thereof such as a PXA1 peroxisomal ATP-binding cassette transporter. This may occur by expression in the plant or part thereof of an exogenous polynucleotide which encodes an RNA molecule which reduces the expression of, for example, an endogenous gene encoding the TAG lipase such as the SDP1 lipase, acyl-CoA oxidase or the polypeptide involved in β-oxidation of fatty acids in the plant or part thereof, or by a mutation in an endogenous gene encoding, for example, the TAG lipase, acyl-CoA oxidase or polypeptide involved in β-oxidation of fatty acids. In an embodiment, the reduced expression and/or activity is relative to a corresponding wild-type plant or part thereof or relative to a corresponding plant or part thereof which has the Push modification.

D. The “Package” modification is characterised by an increased expression and/or accumulation of an oil body coating (OBC) polypeptide. In an embodiment, this can be achieved by expressing in a transgenic plant or part thereof an exogenous polynucleotide which encodes an oil body coating (OBC) polypeptide. The OBC polypeptide may be an oleosin, such as for example a polyoleosin, a caoleosin or a steroleosin, or preferably an LDAP. In an embodiment, the level of oleosin that is accumulated in the plant or part thereof is at least 2-fold higher relative to the corresponding plant or part thereof comprising the oleosin gene from the T-DNA of pJP3502. In an embodiment, the increased expression or accumulation of the OBC polypeptide is not caused solely by the Push modification. In an embodiment, the expression and/or accumulation is relative to a corresponding wild-type plant or part thereof or, preferably, relative to a corresponding plant or part thereof which has the Push modification.

E. The “Plastidial Export” modification is characterised by an increased rate of export of total fatty acids out of the plastids of the plant or part thereof. In one embodiment, this can be achieved by expressing in a plant or part thereof an exogenous polynucleotide which encodes a polypeptide which increases the export of fatty acids out of plastids of the plant or part thereof when compared to a corresponding plant or part thereof lacking the exogenous polynucleotide. In an embodiment, this occurs by the increased expression and/or activity of a fatty acid thioesterase (TE), a fatty acid transporter polypeptide such as an ABCA9 polypeptide, or a long-chain acyl-CoA synthetase (LACS). In an embodiment, the plant or part thereof comprises an exogenous polynucleotide which encodes the TE, fatty acid transporter polypeptide or LACS. The TE may be a FATB polypeptide or preferably a FATA polypeptide. In an embodiment, the TE is preferably a TE which has a preference for hydrolysing MCFA, or MCFA and C16:0 substrates. In an embodiment, the Plastidial Export modification is relative to a corresponding wild-type plant or part thereof or, preferably, relative to a corresponding plant or part thereof which has the Push modification.

F The “Plastidial Import” modification is characterised by a reduced rate of import of fatty acids into the plastids of the plant or part thereof from outside of the plastids. In an embodiment, this can be achieved through a genetic modification in the plant or part thereof which down-regulates endogenous production and/or activity of a polypeptide involved in importing fatty acids into plastids of the plant or part thereof when compared to a corresponding plant or part thereof lacking the genetic modification. For example, this may occur by expression in the plant or part thereof of an exogenous polynucleotide which encodes an RNA molecule which reduces the expression of an endogenous gene encoding an transporter polypeptide such as a TGD polypeptide, for example a TGD1, TGD2, TGD3, TGD4 or preferably a TGD5 polypeptide, or by a mutation in an endogenous gene encoding the TGD polypeptide. In an embodiment, the reduced rate of import is relative to a corresponding wild-type plant or part thereof or relative to a corresponding plant or part thereof which has the Push modification.

G. The “Prokaryotic Pathway” modification is characterised by a decreased amount of DAG or rate of production of DAG in the plastids of the plant or part thereof. In an embodiment, this can be achieved through a genetic modification in the plant or part thereof which down-regulates endogenous production and/or activity of a polypeptide involved in diacylglycerol (DAG) production in the plastid when compared to a corresponding plant or part thereof lacking the genetic modification. In an embodiment, the decreased amount or rate of production of DAG occurs by a decreased production of LPA from acyl-ACP and G3P in the plastids. The decreased amount or rate of production of DAG may occur by expression in the plant or part thereof of an exogenous polynucleotide which encodes an RNA molecule which reduces the expression of an endogenous gene encoding a plastidial GPAT, plastidial LPAAT or a plastidial PAP, preferably a plastidial GPAT, or by a mutation in an endogenous gene encoding the plastidial polypeptide. In an embodiment, the decreased amount or rate of production of DAG is relative to a corresponding wild-type plant or part thereof or, preferably, relative to a corresponding plant or part thereof which has the Push modification.

The Push modification is highly desirable in the invention, and the Pull modification is preferred. The Protect and Package modifications may be complementary i.e. one of the two may be sufficient. The plant or part thereof may comprise one, two or all three of the Plastidial Export, Plastidial Import and Prokaryotic Pathway modifications. In an embodiment, at least one of the exogenous polynucleotides in the plant or part thereof, preferably at least the exogenous polynucleotide encoding the transcription factor which regulates fatty acid synthesis in the plastids, is expressed under the control of (H) a promoter other than a constitutive promoter such as, for example, a developmentally related promoter, a promoter that is preferentially active in photosynthetic cells, a tissue-specific promoter, a promoter which has been modified by reducing its expression level relative to a corresponding native promoter, or is preferably a senesence-specific promoter. More preferably, at least the exogenous polynucleotide encoding the transcription factor which regulates fatty acid synthesis in the plastids is expressed under the control of a promoter other than a constitutive promoter and the exogenous polynucleotide which encodes an RNA molecule which down-regulates endogenous production and/or activity of a polypeptide involved in the catabolism of triacylglycerols is also expressed under the control of a promoter other than a constitutive promoter, which promoters may be the same or different. Alternatively in monocotyledonous plants, the exogenous polynucleotide encoding the transcription factor which regulates fatty acid synthesis in the plastids is expressed under the control of a constitutive promoter such as, for example, a ubiquitin gene promoter or an actin gene promoter.

Plants produce some, but not all, of their membrane lipids such as MGDG in plastids by the so-called prokaryotic pathway (FIG. 1). In plants, there is also a eukaryotic pathway for synthesis of galactolipids and glycerolipids which synthesizes FA first of all in the plastid and then assembles the FA into glycerolipids in the ER. MGDG synthesised by the eukaryotic pathway contains C18:3 (ALA) fatty acid esterified at the sn-2 position of MGDG. The DAG backbone including the ALA for the MGDG synthesis by this pathway is assembled in the ER and then imported into the plastid. In contrast, the MGDG synthesized by the prokaryotic pathway contains C16:3 fatty acid esterified at the sn-2 position of MGDG. The ratio of the contribution of the prokaryotic pathway relative to the eukaryotic pathway in producing MGDG (16:3) vs MGDG (18:3) is a characteristic and distinctive feature of different plant species (Mongrand et al. 1998). This distinctive fatty acid composition of MGDG allows all higher plants (angiosperms) to be classified as either so-called 16:3 or 18:3 plants. 16:3 species, exemplified by Arabidopsis and Brassica napus, generally have both of the prokaryotic and eukaryotic pathways of MGDG synthesis operating, whereas the 18:3 species exemplified by Sorghum bicolor, Zea mays, Nicotiana tabacum, Pisum sativum and Glycine max generally have only (or almost entirely) the eukaryotic pathway of MGDG synthesis, providing little or no C16:3 fatty acid accumulation in the vegetative tissues.

As used herein, a “16:3 plant” or “16:3 species” is one which has more than 2% C16:3 fatty acid in the total fatty acid content of its photosynthetic tissues. As used herein, a “18:3 plant” or “18:3 species” is one which has less than 2% C16:3 fatty acid in the total fatty acid content of its photosynthetic tissues. As described herein, a plant can be converted from being a 16:3 plant to an 18:3 plant by suitable genetic modifications. The proportion of flux between the prokaryote and eukaryote pathways is not conserved across different plant species or tissues. In 16:3 species up to 40% of flux in leaves occurs via the prokaryotic pathway (Browse et al., 1986), while in 18:3 species, such as pea and soybean, about 90% of FAs which are synthesized in the plastid are exported out of the plastid to the ER to supply the source of FA for the eukaryotic pathway (Ohlrogge and Browse, 1995; Somerville et al., 2000).

Therefore different amounts of 18:3 and 16:3 fatty acids are found within the glycolipids of different plant species. This is used to distinguish between 18:3 plants whose fatty acids with 3 double bonds are almost entirely C18 fatty acids and the 16:3 plants that contain both C16- and C18-fatty acids having 3 double bonds. In chloroplasts of 18:3 plants, enzymic activities catalyzing the conversion of phosphatidate to diacylglycerol and of diacylglycerol to monogalactosyl diacylglycerol (MGD) are significantly less active than in 16:3 chloroplasts. In leaves of 18:3 plants, chloroplasts synthesize stearoyl-ACP2 in the stroma, introduce the first double bond into the saturated hydrocarbon chain, and then hydrolyze the thioester by thioesterases (FIG. 1). Released oleate is exported across chloroplast envelopes into membranes of the cell, probably the endoplasmic reticulum, where it is incorporated into PC. PC-linked oleoyl groups are desaturated in these membranes and subsequently move back into the chloroplast. The MGD-linked acyl groups are substrates for the introduction of the third double bond to yield MGD with two linolenoyl residues. This galactolipid is characteristic of 18:3 plants such as Asteraceae and Fabaceae, for example. In photosynthetically active cells of 16:3 plants which are represented, for example, by members of Apiaceae and Brassicaceae, two pathways operate in parallel to provide thylakoids with MGD.

In one embodiment, the plant or part thereof such as a vegetative plant part of the invention produces higher levels of non-polar lipids such as TAG, or MFCA content, preferably both, than a corresponding plant or part thereof such as a vegetative plant part which lacks the genetic modifications or exogenous polynucleotides. In one example, plants of the invention produce seeds, leaves, or have leaf portions of at least 1 cm² in surface area, stems and/or tubers having an increased non-polar lipid content such as TAG or MCFA content, preferably both, when compared to corresponding seeds, leaves, leaf portions of at least 1 cm² in surface area, stems or tubers.

Preferably, the plant or part thereof such as a vegetative plant part of the invention is transformed with one or more exogenous polynucleotides such as chimeric DNAs. In the case of multiple chimeric DNAs, these are preferably covalently linked on one DNA molecule such as, for example, a single T-DNA molecule, and preferably integrated at a single locus in the host cell genome, preferably the host nuclear genome. Alternatively, the chimeric DNAs are on two or more DNA molecules which may be unlinked in the host genome, or the DNA molecule(s) is not integrated into the host genome, such as occurs in transient expression experiments. The plant or part thereof such as a vegetative plant part is preferably homozygous for the one DNA molecule inserted into its genome.

### Transcription Factors

Various transcription factors are involved in plant cells in the synthesis of fatty acids and lipids incorporating the fatty acids such as TAG, and therefore can be manipulated for the Push modification. A preferred transcription factor is WRI1. As used herein, the term “Wrinkled 1” or “WRI1” or “WRL1” refers to a transcription factor of the AP2/ERWEBP class which regulates the expression of several enzymes involved in glycolysis and de novo fatty acid biosynthesis. WRI1 has two plant-specific (AP2/EREB) DNA-binding domains. WRI1 in at least Arabidopsis also regulates the breakdown of sucrose via glycolysis thereby regulating the supply of precursors for fatty acid biosynthesis. In other words, it controls the carbon flow from the photosynthate to storage lipids. wril mutants in at least Arabidopsis have a wrinkled seed phenotype, due to a defect in the incorporation of sucrose and glucose into TAGs.

Examples of genes which are transcribed by WRI1 include, but are not limited to, one or more, preferably all, of genes encoding pyruvate kinase (At5 g52920, At3 g22960), pyruvate dehydrogenase (PDH) E1alpha subunit (At1 g01090), acetyl-CoA carboxylase (ACCase), BCCP2 subunit (At5 g15530), enoyl-ACP reductase (At2 g05990; EAR), phosphoglycerate mutase (At1 g22170), cytosolic fructokinase, and cytosolic phosphoglycerate mutase, sucrose synthase (SuSy) (see, for example, Liu et al., 2010; Baud et al., 2007; Ruuska et al., 2002).

WRI1 contains the conserved domain AP2 (cd00018). AP2 is a DNA-binding domain found in transcription regulators in plants such as APETALA2 and EREBP (ethylene responsive element binding protein). In EREBPs the domain specifically binds to the 11 bp GCC box of the ethylene response element (ERE), a promotor element essential for ethylene responsiveness. EREBPs and the C-repeat binding factor CBF1, which is involved in stress response, contain a single copy of the AP2 domain. APETALA2-like proteins, which play a role in plant development contain two copies.

Other sequence motifs which may be found in WRI1 and its functional homologs include:

SEQ ID NO: 14
1. R G V T/S R H R W T G R.
SEQ ID NO: 15
2. F/Y E A H L W D K.
SEQ ID NO: 16
3. D L A A L K Y W G.
SEQ ID NO: 17
4. S X G F S/A R G X.
SEQ ID NO: 18
5. H H H/Q N G R/K W E A R I G R/K V.
SEQ ID NO: 19
6. Q E E A A A X Y D.

As used herein, the term “Wrinkled 1” or “WRI1” also includes “Wrinkled 1-like” or “WRI1-like” proteins. Examples of WRI1 proteins include Accession Nos: A8MS57 (Arabidopsis thaliana), Q6X5Y6, (Arabidopsis thaliana), XP_002876251.1 (Arabidopsis lyrata subsp. Lyrata), ABD16282.1 (Brassica napus), ADO16346.1 (Brassica napus), XP_003530370.1 (Glycine max), AEO22131.1 (Jatropha curcas), XP_002525305.1 (Ricinus communis), XP_002316459.1 (Populus trichocarpa), CBI29147.3 (Vitis vinifera), XP_003578997.1 (Brachypodium distachyon), BAJ86627.1 (Hordeum vulgare subsp. vulgare), EAY79792.1 (Oryza sativa), XP_002450194.1 (Sorghum bicolor), ACG32367.1 (Zea mays), XP_003561189.1 (Brachypodium distachyon), ABL85061.1 (Brachypodium sylvaticum), BAD68417.1 (Oryza sativa), XP_002437819.1 (Sorghum bicolor), XP 002441444.1 (Sorghum bicolor), XP_003530686.1 (Glycine max), XP_003553203.1 (Glycine max), XP_002315794.1 (Populus trichocarpa), XP_002270149.1 (Vitis vinifera), XP_003533548.1 (Glycine max), XP_003551723.1 (Glycine max), XP_003621117.1 (Medicago truncatula), XP_002323836.1 (Populus trichocarpa), XP_002517474.1 (Ricinus communis), CAN79925.1 (Vitis vinifera), XP_003572236.1 (Brachypodium distachyon), BAD10030.1 (Oryza sativa), XP_002444429.1 (Sorghum bicolor), NP_001170359.1 (Zea mays), XP_002889265.1 (Arabidopsis lyrata subsp. lyrata), AAF68121.1 (Arabidopsis thaliana), NP_178088.2 (Arabidopsis thaliana), XP 002890145.1 (Arabidopsis lyrata subsp. lyrata), BAJ33872.1 (Thellungiella halophila), NP_563990.1 (Arabidopsis thaliana), XP_003530350.1 (Glycine max), XP_003578142.1 (Brachypodium distachyon), EAZ09147.1 (Oryza sativa), XP_002460236.1 (Sorghum bicolor), NP_001146338.1 (Zea mays), XP_003519167.1 (Glycine max), XP_003550676.1 (Glycine max), XP 003610261.1 (Medicago truncatula), XP_003524030.1 (Glycine max), XP_003525949.1 (Glycine max), XP_002325111.1 (Populus trichocarpa), CBI36586.3 (Vitis vinifera), XP 002273046.2 (Vitis vinifera), XP_002303866.1 (Populus trichocarpa), and CBI25261.3 (Vitis vinifera). Further examples include Sorbi-WRL1 (SEQ ID NO:10), Lupan-WRL1 (SEQ ID NO:11), Ricco-WRL1 (SEQ ID NO:12), and Lupin angustifolius WRI1 (SEQ ID NO:13). A preferred WRI1 is a maize WRI1 or a sorghum WRI1. In an embodiment, an exogenous polynucleotide of the invention which encodes a WRI1 which comprises one or more of the following:
i) nucleotides encoding a polypeptide comprising amino acids whose sequence is set forth in any of the above mentioned accessions, or a biologically active fragment thereof, or a polypeptide whose amino acid sequence is at least 30% identical to that set forth in any of the above mentioned accessions,
ii) nucleotides whose sequence is at least 30% identical to i), and
iii) a polynucleotide which hybridizes to one or both of i) or ii) under stringent conditions.

More recently, a subset of WRI1-like transcription factors have been re-classified as WRI2, WRI3 or WRI4 transcription factors, which are characterised by preferential expression in stems and/or roots of plants rather than in developing seeds (To et al., 2012). Despite their re-classification, these are included in the definition of “WRI1” herein. Preferred WRI-like transcription factors are those which can complement the function of a wril mutation in a plant, particularly the function in developing seed of the plant such as in an A. thaliana wril mutant. The function of a WRI1-like polypeptide can also be assayed in the N. benthamiana transient assays as described herein.

The WRI1 transcription factor may be endogenous to the plant or cell, or exogenous to the plant or cell, for example expressed from an exogenous polynucleotide. The WRI1 transcription factor may be a naturally occurring WRI1 polypeptide or a variant thereof, provided it retains transcription factor activity. The level or activity of an endogenous WRI1 polypeptide may also be increased by increased expression of a MED15 polypeptide (Kim et al., 2016), for example polypeptides whose amino acid sequences are provided in Accession No: NM_101446.4 or NM_001321633.1, or of a 14-3-3 polypeptide (Ma et al., 2016), for example Accession Nos: AY079350, AY079350, XM_002445734.1, XM_002445734.1, NM_001203346, NM_001203346, XM_002445734.1, or XM_002445734.1. MED15 polypeptide is thought to assist in directing WRI1 to its target promoters and expression of WRI1 expression itself, while 14-3-3 polypeptides are thought to interact with WRI1 polypeptide to increase the WRI1 effect.

As used herein, a “LEAFY COTYLEDON” or “LEC” polypeptide means a transcription factor which is a LEC1, LEC1-like, LEC2, ABI3 or FUS3 transcription factor which exhibits broad control on seed maturation and fatty acid synthesis. LEC2, FUS3 and ABI3 are related polypeptides that each contain a B3 DNA-binding domain of 120 amino acids (Yamasaki et al., 2004) that is only found in plant proteins. They can be distinguished by phylogenetic analysis to determine relatedness in amino acid sequence to the members of the A. thaliana polypeptides having the Accession Nos as follows: LEC2, Accession No. AAL12004.1; FUS3 (also known as FUSCA3), Accession No. AAC35247. LEC1 belongs to a different class of polypeptides and is homologous to a HAP3 polypeptide of the CBF binding factor class (Lee et al., 2003). The LEC1, LEC2 and FUS3 genes are required in early embryogenesis to maintain embryonic cell fate and to specify cotyledon identity and in later in initiation and maintenance of embryo maturation (Santos-Mendoza et al., 2008). They also induce expression of genes encoding seed storage proteins by binding to RY motifs present in the promoters, and oleosin genes. They can also be distinguished by their expression patterns in seed development or by their ability to complement the corresponding mutation in A. thaliana.

As used herein, the term “Leafy Cotyledon 1” or “LEC1” refers to a NF-YB-type transcription factor which participates in zygotic development and in somatic embryogenesis. The endogenous gene is expressed specifically in seed in both the embryo and endosperm. LEC1 activates the gene encoding WRI1 as well as a large class of fatty acid synthesis genes. Ectopic expression of LEC2 also causes rapid activation of auxin-responsive genes and may cause formation of somatic embryos. Examples of LEC1 polypeptides include proteins from Arabidopsis thaliana (AAC39488, SEQ ID NO:31), Medicago truncatula (AFK49653) and Brassica napus (ADF81045), A. lyrata (XP_002862657), R. communis (XP_002522740), G. max (XP_006582823), A. hypogaea (ADC33213), Z. mays (AAK95562, SEQ ID NO:32). In an embodiment, an exogenous polynucleotide of the invention which encodes a LEC1 which comprises one or more of the following:
i) nucleotides encoding a polypeptide comprising amino acids whose sequence is set forth in any of the above mentioned accessions, or a biologically active fragment thereof, or a polypeptide whose amino acid sequence is at least 30% identical to that set forth in any of the above mentioned accessions,
ii) nucleotides whose sequence is at least 30% identical to i), and
iii) a polynucleotide which hybridizes to one or both of i) or ii) under stringent conditions. LEC1-like (LIL) is closely related to LEC1 but has a different pattern of gene expression, being expressed earlier during embryogenesis (Kwong et al., 2003). Examples of LEC1-like polypeptides include proteins from Arabidopsis thaliana (AAN15924, SEQ ID NO:33), Brassica napus (AHI94922), and Phaseolus coccineus LEC1-like (AAN01148).

As used herein, the term “Leafy Cotyledon 2” or “LEC2” refers to a B3 domain transcription factor which participates in zygotic development and in somatic embryogenesis and which activates expression of a gene encoding WRI1. Its ectopic expression facilitates the embryogenesis from vegetative plant tissues (Alemanno et al., 2008). Examples of LEC2 polypeptides include proteins from Arabidopsis thaliana (Accession No. NP_564304.1), Medicago truncatula (Accession No. CAA42938.1) and Brassica napus (Accession No. ADO16343.1). In an embodiment, an exogenous polynucleotide of the invention which encodes a LEC2 which comprises one or more of the following:
i) nucleotides encoding a polypeptide comprising amino acids whose sequence is set forth in any of the above mentioned accessions, or a biologically active fragment thereof, or a polypeptide whose amino acid sequence is at least 30% identical to that set forth in any of the above mentioned accessions,
ii) nucleotides whose sequence is at least 30% identical to i), and
iii) a polynucleotide which hybridizes to one or both of i) or ii) under stringent conditions.

As used herein, the term “FUS3” refers to a B3 domain transcription factor which participates in zygotic development and in somatic embryogenesis and is detected mainly in the protodermal tissue of the embryo (Gazzarrini et al., 2004). Examples of FUS3 polypeptides include proteins from Arabidopsis thaliana (AAC35247, SEQ ID NO:34), Brassica napus (XP_006293066.1, SEQ ID NO:35) and Medicago truncatula (XP_003624470, SEQ ID NO:36). Over-expression of any of LEC1, LIL, LEC2, FUS3 and ABI3 from an exogenous polynucleotide is preferably controlled by a developmentally regulated promoter such as a senescence specific promoter, an inducible promoter, or a promoter which has been engineered for providing a reduced level of expression relative to a native promoter, particularly in plants other than Arabidopsis thaliana and B. napus cv. Westar, in order to avoid developmental abnormalities in plant development that are commonly associated with over-expression of these transcription factors (Mu et al., 2008). In an embodiment, an exogenous polynucleotide of the invention which encodes a FUS3 which comprises one or more of the following:
i) nucleotides encoding a polypeptide comprising amino acids whose sequence is set forth in any of the above mentioned accessions, or a biologically active fragment thereof, or a polypeptide whose amino acid sequence is at least 30% identical to that set forth in any of the above mentioned accessions,
ii) nucleotides whose sequence is at least 30% identical to i), and
iii) a polynucleotide which hybridizes to one or both of i) or ii) under stringent conditions.

As used herein, the term “BABY BOOM” or “BBM” refers an AP2/ERF transcription factor that induces regeneration under culture conditions that normally do not support regeneration in wild-type plants. Ectopic expression of Brassica napus BBM (BnBBM) genes in B. napus and Arabidopsis induces spontaneous somatic embryogenesis and organogenesis from seedlings grown on hormone-free basal medium (Boutilier et al., 2002). In tobacco, ectopic BBM expression is sufficient to induce adventitious shoot and root regeneration on basal medium, but exogenous cytokinin is required for somatic embryo (SE) formation (Srinivasan et al., 2007). Examples of BBM polypeptides include proteins from Arabidopsis thaliana (Accession No. NP_197245.2, SEQ ID NO:28), maize (U.S. Pat. No. 7,579,529), Sorghum bicolor (Accession No. XP_002458927) and Medicago truncatula (Accession No. AAW82334.1). In an embodiment, an exogenous polynucleotide of the invention which encodes a BBM which comprises one or more of the following:
i) nucleotides encoding a polypeptide comprising amino acids whose sequence is set forth in any of the above mentioned accessions, or a biologically active fragment thereof, or a polypeptide whose amino acid sequence is at least 30% identical to that set forth in any of the above mentioned accessions,
ii) nucleotides whose sequence is at least 30% identical to i), and
iii) a polynucleotide which hybridizes to one or both of i) or ii) under stringent conditions.

An ABI3 polypeptide (A. thaliana Accession No. NP_189108) is related to the maize VP1 protein, is expressed at low levels in vegetative tissues and affects plastid development. An ABI4 polypeptide (A. thaliana Accession NP_181551) belongs to a family of transcription factors that contain a plant-specific AP2 domain (Finkelstein et al., 1998) and acts downstream of ABI3. ABI5 (A. thaliana Accession No. NP_565840) is a transcription factor of the bZIP family which affects ABA sensitivity and controls the expression of some LEA genes in seeds. It binds to an ABA-responsive element.

Each of the following transcription factors was selected on the basis that they functioned in embryogenesis in plants. Accession numbers are provided in Table 8. Homologs of each can be readily identified in many other plant species and tested as described in Example 4.

MYB73 is a transcription factor that has been identified in soybean, involved in stress responses.

bZIP53 is a transcription factor in the bZIP protein family, identified in Arabidopsis.

AGL15 (Agamous-like 15) is a MADS box transcription factor which is natively expressed during embryogenesis. AGL15 is also natively expressed in leaf primordia, shoot apical meristems and young floral buds, suggesting that AGL15 may also have a function during post-germinative development. AGL15 has a role in embryogenesis and gibberellic acid catabolism. It targets B3 domain transcription factors that are key regulators of embryogenesis.

MYB115 and MYB118 are transcription factors in the MYB family from Arabidopsis involved in embryogenesis.

TANMEI also known as EMB2757 encodes a WD repeat protein required for embryo development in Arabidopsis.

WUS, also known as Wuschel, is a homeobox gene that controls the stem cell pool in embryos. It is expressed in the stem cell organizing center of meristems and is required to keep the stem cells in an undifferentiated state. The transcription factor binds to a TAAT element core motif.

GFR2a1 and GFR2a2 are transcription factors at least from soybean.

### Fatty Acyl Acyltransferases

As used herein, the term “fatty acyl acyltransferase” refers to a protein which is capable of transferring an acyl group from acyl-CoA, PC or acyl-ACP, preferably acyl-CoA or PC, onto a substrate to form TAG, DAG or MAG. These acyltransferases include DGAT, PDAT, MGAT, GPAT and LPAAT.

As used herein, the term “diacylglycerol acyltransferase” (DGAT) refers to a protein which transfers a fatty acyl group from acyl-CoA to a DAG substrate to produce TAG. Thus, the term “diacylglycerol acyltransferase activity” refers to the transfer of an acyl group from acyl-CoA to DAG to produce TAG. A DGAT may also have MGAT function but predominantly functions as a DGAT, i.e., it has greater catalytic activity as a DGAT than as a MGAT when the enzyme activity is expressed in units of nmoles product/min/mg protein (see for example, Yen et al., 2005). The activity of DGAT may be rate-limiting in TAG synthesis in seeds (Ichihara et al., 1988). DGAT uses an acyl-CoA substrate as the acyl donor and transfers it to the sn-3 position of DAG to form TAG. The enzyme functions in its native state in the endoplasmic reticulum (ER) of the cell.

There are three known types of DGAT, referred to as DGAT1, DGAT2 and DGAT3, respectively. DGAT1 polypeptides are membrane proteins that typically have 10 transmembrane domains, DGAT2 polypeptides are also membrane proteins but typically have 2 transmembrane domains, whilst DGAT3 polypeptides typically have none and are thought to be soluble in the cytoplasm, not integrated into membranes. Plant DGAT1 polypeptides typically have about 510-550 amino acid residues while DGAT2 polypeptides typically have about 310-330 residues. DGAT1 is the main enzyme responsible for producing TAG from DAG in most developing plant seeds, whereas DGAT2s from plant species such as tung tree (Vernicia fordii) and castor bean (Ricinus communis) that produce high amounts of unusual fatty acids appear to have important roles in the accumulation of the unusual fatty acids in TAG. Over-expression of AtDGAT1 in tobacco leaves resulted in a 6-7 fold increased TAG content (Bouvier-Nave et al., 2000).

Examples of DGAT1 polypeptides include DGAT1 proteins from Aspergillus fumigatus (XP_755172.1), Arabidopsis thaliana (CAB44774.1; SEQ ID NO:1), Ricinus communis (AAR11479.1), Vernicia fordii (ABC94472.1), Vernonia galamensis (ABV21945.1 and ABV21946.1), Euonymus alatus (AAV31083.1), Caenorhabditis elegans (AAF82410.1), Rattus norvegicus (NP_445889.1), Homo sapiens (NP_036211.2), as well as variants and/or mutants thereof. In an embodiment, an exogenous polynucleotide of the invention which encodes a DGAT1 which comprises one or more of the following:
i) nucleotides encoding a polypeptide comprising amino acids whose sequence is set forth in any of the above mentioned accessions, or a biologically active fragment thereof, or a polypeptide whose amino acid sequence is at least 30% identical to that set forth in any of the above mentioned accessions,
ii) nucleotides whose sequence is at least 30% identical to i), and
iii) a polynucleotide which hybridizes to one or both of i) or ii) under stringent conditions.

Examples of DGAT2 polypeptides include proteins encoded by DGAT2 genes from Arabidopsis thaliana (NP_566952.1), Ricinus communis (AAY16324.1), Vernicia fordii (ABC94474.1), Mortierella ramanniana (AAK84179.1), Homo sapiens (Q96PD7.2) (Q58HT5.1), Bos taurus (Q70VZ8.1), Mus musculus (AAK84175.1), as well as variants and/or mutants thereof. DGAT1 and DGAT2 amino acid sequences show little homology. Expression in leaves of an exogenous DGAT2 was twice as effective as a DGAT1 in increasing oil content (TAG). Further, A. thaliana DGAT2 had a greater preference for linoleoyl-CoA and linolenoyl-CoA as acyl donors relative to oleoyl-CoA, compared to DGAT1. This substrate preference can be used to distinguish the two DGAT classes in addition to their amino acid sequences. In an embodiment, an exogenous polynucleotide of the invention which encodes a DGAT2 which comprises one or more of the following:
i) nucleotides encoding a polypeptide comprising amino acids whose sequence is set forth in any of the above mentioned accessions, or a biologically active fragment thereof, or a polypeptide whose amino acid sequence is at least 30% identical to that set forth in any of the above mentioned accessions,
ii) nucleotides whose sequence is at least 30% identical to i), and
iii) a polynucleotide which hybridizes to one or both of i) or ii) under stringent conditions.

Examples of DGAT3 polypeptides include proteins encoded by DGAT3 genes from peanut (Arachis hypogaea, Saha, et al., 2006), as well as variants and/or mutants thereof. A DGAT has little or no detectable MGAT activity, for example, less than 300 pmol/min/mg protein, preferably less than 200 pmol/min/mg protein, more preferably less than 100 pmol/min/mg protein.

In a particularly preferred embodiment, the DGAT has a preference for medium chain fatty acids. For instance, the DGAT comprises one or more of the following:
i) nucleotides encoding a polypeptide comprising amino acids whose sequence is set forth in
SEQ ID NO:56, or a biologically active fragment thereof, or a polypeptide whose amino acid sequence is at least 30% identical to SEQ ID NO:56,
ii) nucleotides whose sequence is at least 30% identical to i), and
iii) a polynucleotide which hybridizes to one or both of i) or ii) under stringent conditions.

As used herein, the term “phospholipid:diacylglycerol acyltransferase” (PDAT; EC 2.3.1.158) or its synonym “phospholipid: 1,2-diacyl-sn-glycerol O-acyltransferase” means an acyltransferase that transfers an acyl group from a phospholipid, typically PC, to the sn-3 position of DAG to form TAG. This reaction is different to DGAT and uses phospholipids as the acyl-donors. Increased expression of PDAT such as PDAT1, which may be exogenous or endogenous to the cell or plant of the invention, increases the production of TAG from PC. There are several forms of PDAT in plant cells including PDAT1, PDAT2 or PDAT3 (Ghosal et al., 2007). Sequences of exemplary PDAT coding regions and polypeptides are provided in Accession Nos: XM_002462417.1, (Sorghum), NM_001147943 (Zea mays), (Dahlqvist et al., 2000; Fan et al., 2013a and b; Fan et al., 2014) although any PDAT encoding gene can be used. The PDAT may be exogenous or endogenous to the plant or part thereof.

As used herein, the term “monoacylglycerol acyltransferase” or “MGAT” refers to a protein which transfers a fatty acyl group from acyl-CoA to a MAG substrate, for example sn-2 MAG, to produce DAG. Thus, the term “monoacylglycerol acyltransferase activity” at least refers to the transfer of an acyl group from acyl-CoA to MAG to produce DAG. The term “MGAT” as used herein includes enzymes that act on sn-1/3 MAG and/or sn-2 MAG substrates to form sn-1,3 DAG and/or sn-1,2/2,3-DAG, respectively. In a preferred embodiment, the MGAT has a preference for sn-2 MAG substrate relative to sn-1 MAG, or substantially uses only sn-2 MAG as substrate. As used herein, MGAT does not include enzymes which transfer an acyl group preferentially to LysoPA relative to MAG, such enzymes are known as LPAATs. That is, a MGAT preferentially uses non-phosphorylated monoacyl substrates, even though they may also have low catalytic activity on LysoPA. A preferred MGAT does not have detectable activity in acylating LysoPA. A MGAT may also have DGAT function but predominantly functions as a MGAT, i.e., it has greater catalytic activity as a MGAT than as a DGAT when the enzyme activity is expressed in units of nmoles product/min/mg protein (also see Yen et al., 2002). There are three known classes of MGAT, referred to as, MGAT1, MGAT2 and MGAT3, respectively. Examples of MGAT1, MGAT2 and MGAT3 polypeptides are described in WO2013/096993.

As used herein, an “MGAT pathway” refers to an anabolic pathway, different to the Kennedy pathway for the formation of TAG, in which DAG is formed by the acylation of either sn-1 MAG or preferably sn-2 MAG, catalysed by MGAT. The DAG may subsequently be used to form TAG or other lipids. WO2012/000026 demonstrated firstly that plant leaf tissue can synthesise MAG from G-3-P such that the MAG is accessible to an exogenous MGAT expressed in the leaf tissue, secondly MGAT from various sources can function in plant tissues, requiring a successful interaction with other plant factors involved in lipid synthesis and thirdly the DAG produced by the exogenous MGAT activity is accessible to a plant DGAT, or an exogenous DGAT, to produce TAG. MGAT and DGAT activity can be assayed by introducing constructs encoding the enzymes (or candidate enzymes) into Saccharomyces cerevisiae strain H1246 and demonstrating TAG accumulation.

Some of the motifs that have been shown to be important for catalytic activity in some DGAT2s are also conserved in MGAT acyltransferases. Of particular interest is a putative neutral lipid-binding domain with the concensus sequence FLXLXXXN (SEQ ID NO:6) where each X is independently any amino acid other than proline, and N is any nonpolar amino acid, located within the N-terminal transmembrane region followed by a putative glycerol/phospholipid acyltransferase domain. The FLXLXXXN motif (SEQ ID NO:6) is found in the mouse DGAT2 (amino acids 81-88) and MGAT1/2 but not in yeast or plant DGAT2s. It is important for activity of the mouse DGAT2. Other DGAT2 and/or MGAT1/2 sequence motifs include:
1. A highly conserved YFP tripeptide (SEQ ID NO:2) in most DGAT2 polypeptides and also in MGAT1 and MGAT2, for example, present as amino acids 139-141 in mouse DGAT2. Mutating this motif within the yeast DGAT2 with non-conservative substitutions rendered the enzyme non-functional.
2. HPHG tetrapeptide (SEQ ID NO:3), highly conserved in MGATs as well as in DGAT2 sequences from animals and fungi, for example, present as amino acids 161-164 in mouse DGAT2, and important for catalytic activity at least in yeast and mouse DGAT2. Plant DGAT2 acyltransferases have a EPHS (SEQ ID NO:4) conserved sequence instead, so conservative changes to the first and fourth amino acids can be tolerated.
3. A longer conserved motif which is part of the putative glycerol phospholipid domain. An example of this motif is
RXGFX(K/R)XAXXXGXXX(L/V)VPXXXFG(E/Q) (SEQ ID NO:5), which is present as amino acids 304-327 in mouse DGAT2. This motif is less conserved in amino acid sequence than the others, as would be expected from its length, but homologs can be recognised by motif searching. The spacing may vary between the more conserved amino acids, i.e., there may be additional X amino acids within the motif, or less X amino acids compared to the sequence above.

One important component in glycerolipid synthesis from fatty acids esterified to ACP or CoA is the enzyme sn-glycerol-3-phosphate acyltransferase (GPAT), which is another of the polypeptides involved in the biosynthesis of non-polar lipids. This enzyme is involved in different metabolic pathways and physiological functions. It catalyses the following reaction: G3P+fatty acyl-ACP or -CoA→LPA+free-ACP or -CoA. The GPAT-catalyzed reaction occurs in three distinct plant subcellular compartments: plastid, endoplasmic reticulum (ER) and mitochondria. These reactions are catalyzed by three different types of GPAT enzymes, a soluble form localized in plastidial stroma which uses acyl-ACP as its natural acyl substrate (PGPAT in FIG. 1), and two membrane-bound forms localized in the ER and mitochondria which use acyl-CoA and acyl-ACP as natural acyl donors, respectively (Chen et al., 2011).

As used herein, the term “glycerol-3-phosphate acyltransferase” (GPAT; EC 2.3.1.15) and its synonym “glycerol-3-phosphate O-acyltransferase” refer to a protein which acylates glycerol-3-phosphate (G-3-P) to form LysoPA and/or MAG, the latter product forming if the GPAT also has phosphatase activity on LysoPA. The acyl group that is transferred is from acyl-CoA if the GPAT is an ER-type GPAT (an “acyl-CoA:sn-glycerol-3-phosphate 1-O-acyltransferase” also referred to as “microsomal GPAT”) or from acyl-ACP if the GPAT is a plastidial-type GPAT (PGPAT). Thus, the term “glycerol-3-phosphate acyltransferase activity” refers to the acylation of G-3-P to form LysoPA and/or MAG. The term “GPAT” encompasses enzymes that acylate G-3-P to form sn-1 LPA and/or sn-2 LPA, preferably sn-2 LPA. Preferably, the GPAT which may be over-expressed in the Pull modification is a membrane bound GPAT that functions in the ER of the cell, more preferably a GPAT9, and the plastidial GPAT that is down-regulated in the Prokaryotic Pathway modification is a soluble GPAT (“plastidial GPAT”). In a preferred embodiment, the GPAT has phosphatase activity. In a most preferred embodiment, the GPAT is a sn-2 GPAT having phosphatase activity which produces sn-2 MAG.

As used herein, the term “sn-1 glycerol-3-phosphate acyltransferase” (sn-1 GPAT) refers to a protein which acylates sn-glycerol-3-phosphate (G-3-P) to preferentially form 1-acyl-sn-glycerol-3-phosphate (sn-1 LPA). Thus, the term “sn-1 glycerol-3-phosphate acyltransferase activity” refers to the acylation of sn-glycerol-3-phosphate to form 1-acyl-sn-glycerol-3-phosphate (sn-1 LPA).

As used herein, the term “sn-2 glycerol-3-phosphate acyltransferase” (sn-2 GPAT) refers to a protein which acylates sn-glycerol-3-phosphate (G-3-P) to preferentially form 2-acyl-sn-glycerol-3-phosphate (sn-2 LPA). Thus, the term “sn-2 glycerol-3-phosphate acyltransferase activity” refers to the acylation of sn-glycerol-3-phosphate to form 2-acyl-sn-glycerol-3-phosphate (sn-2 LPA).

The GPAT family is large and all known members contain two conserved domains, a plsC acyltransferase domain (PF01553) and a HAD-like hydrolase (PF12710) superfamily domain and variants thereof. In addition to this, at least in Arabidopsis thaliana, GPATs in the subclasses GPAT4-GPAT8 all contain a N-terminal region homologous to a phosphoserine phosphatase domain (PF00702), and GPATs which produce MAG as a product can be identified by the presence of such a homologous region. Some GPATs expressed endogenously in leaf tissue comprise the conserved amino acid sequence GDLVICPEGTTCREP (SEQ ID NO:7). GPAT4 and GPAT6 both contain conserved residues that are known to be critical to phosphatase activity, specifically conserved amino acids in Motif I (DXDX[T/V][L/V]; SEQ ID NO:8) and Motif III (K-[G/S][D/S]XXX[D/N]; SEQ ID NO:9) located at the N-terminus (Yang et al., 2010).

Homologues of Arabidopsis GPAT4 (Accession No. NP_171667.1) and GPAT6 (NP_181346.1) include AAF02784.1 (Arabidopsis thaliana), AAL32544.1 (Arabidopsis thaliana), AAP03413.1 (Oryza sativa), ABK25381.1 (Picea sitchensis), ACN34546.1 (Zea Mays), BAF00762.1 (Arabidopsis thaliana), BAH00933.1 (Oryza sativa), EAY84189.1 (Oryza sativa), EAY98245.1 (Oryza sativa), EAZ21484.1 (Oryza sativa), EEC71826.1 (Oryza sativa), EEC76137.1 (Oryza sativa), EEE59882.1 (Oryza sativa), EFJ08963.1 (Selaginella moellendorffii), EFJ11200.1 (Selaginella moellendorffii), NP_001044839.1 (Oryza sativa), NP_001045668.1 (Oryza sativa), NP_001147442.1 (Zea mays), NP_001149307.1 (Zea mays), NP_001168351.1 (Zea mays), AFH02724.1 (Brassica napus) NP_191950.2 (Arabidopsis thaliana), XP_001765001.1 (Physcomitrella patens), XP_001769671.1 (Physcomitrella patens), (Vitis vinifera), XP_002275348.1 (Vitis vinifera), XP_002276032.1 (Vitis vinifera), XP_002279091.1 (Vitis vinifera), XP_002309124.1 (Populus trichocarpa), XP_002309276.1 (Populus trichocarpa), XP_002322752.1 (Populus trichocarpa), XP_002323563.1 (Populus trichocarpa), XP_002439887.1 (Sorghum bicolor), XP_002458786.1 (Sorghum bicolor), XP_002463916.1 (Sorghum bicolor), XP_002464630.1 (Sorghum bicolor), XP 002511873.1 (Ricinus communis), XP_002517438.1 (Ricinus communis), XP_002520171.1 (Ricinus communis), ACT32032.1 (Vernicia fordii), NP_001051189.1 (Oryza sativa), AFH02725.1 (Brassica napus), XP_002320138.1 (Populus trichocarpa), XP_002451377.1 (Sorghum bicolor), XP_002531350.1 (Ricinus communis), and XP_002889361.1 (Arabidopsis lyrata).

In an embodiment, an exogenous polynucleotide of the invention which encodes a GPAT which comprises one or more of the following:
i) nucleotides encoding a polypeptide comprising amino acids whose sequence is set forth in any of the above mentioned accessions, or a biologically active fragment thereof, or a polypeptide whose amino acid sequence is at least 30% identical to that set forth in any of the above mentioned accessions,
ii) nucleotides whose sequence is at least 30% identical to i), and
iii) a polynucleotide which hybridizes to one or both of i) or ii) under stringent conditions.

In a particularly preferred embodiment, the GPAT, preferably a GPAT9, has a preference for utilising medium chain fatty acid substrates. For instance, the GPAT9 comprises one or more of the following:
i) nucleotides encoding a polypeptide comprising amino acids whose sequence is set forth in any of SEQ ID NO:97 to 119, preferably SEQ ID NO:97, or a biologically active fragment thereof, or a polypeptide whose amino acid sequence is at least 30% identical to any one of SEQ ID NO:97 to 119, preferably at least 30% identical to SEQ ID NO:97,
ii) nucleotides whose sequence is at least 30% identical to i), and
iii) a polynucleotide which hybridizes to one or both of i) or ii) under stringent conditions.

The soluble plastidial GPATs (PGPAT, also known as ATS1 in Arabidopsis thaliana) have been purified and genes encoding them cloned from several plant species such as pea (Pisum sativum, Accession number: P30706.1), spinach (Spinacia oleracea, Accession number: Q43869.1), squash (Cucurbita moschate, Accession number: P10349.1), cucumber (Cucumis sativus, Accession number: Q39639.1) and Arabidopsis thaliana (Accession number: Q43307.2). The soluble plastidial GPAT is the first committed step for what is known as the prokaryotic pathway of glycerolipid synthesis and is operative only in the plastid (FIG. 1). The so-called prokaryotic pathway is located exclusively in plant plastids and assembles DAG for the synthesis of galactolipids (MGDG and DGMG) which contain C16:3 fatty acids esterified at the sn-2 position of the glycerol backbone.

Conserved motifs and/or residues can be used as a sequence-based diagnostic for the identification of GPAT enzymes. Alternatively, a more stringent function-based assay could be utilised. Such an assay involves, for example, feeding labelled glycerol-3-phosphate to cells or microsomes and quantifying the levels of labelled products by thin-layer chromatography or a similar technique. GPAT activity results in the production of labelled LPA whilst GPAT/phosphatase activity results in the production of labelled MAG.

As used herein, the term “lysophosphatidic acid acyltransferase” (LPAAT; EC 2.3.1.51) and its synonyms “1-acyl-glycerol-3-phosphate acyltransferase”, “acyl-CoA: 1-acyl-sn-glycerol-3-phosphate 2-O-acyltransferase” and “1-acylglycerol-3-phosphate O-acyltransferase” refer to a protein which acylates lysophosphatidic acid (LPA) to form phosphatidic acid (PA). The acyl group that is transferred is from acyl-CoA if the LPAAT is an ER-type LPAAT or from acyl-ACP if the LPAAT is a plastidial-type LPAAT (PLPAAT). Thus, the term “lysophosphatidic acid acyltransferase activity” refers to the acylation of LPA to form PA.

In a particularly preferred embodiment, the LPAAT has a preference for medium chain fatty acids. For instance, the LPAAT comprises one or more of the following:
i) nucleotides encoding a polypeptide comprising amino acids whose sequence is set forth in SEQ ID NO:94, or a biologically active fragment thereof, or a polypeptide whose amino acid sequence is at least 30% identical to SEQ ID NO:94,
ii) nucleotides whose sequence is at least 30% identical to i), and
iii) a polynucleotide which hybridizes to one or both of i) or ii) under stringent conditions.

### Oil Body Coating Polypeptides

TAGs are accumulated in plant tissues as subcellular spherical lipid droplets (LDs, also called oil bodies or lipid bodies) of approximately 0.5-2 μm in diameter. In seeds, each LD has a matrix of TAGs surrounded by a layer of phospholipids (PLs) and structural proteins termed oleosins (Chapman and Ohlrogge, 2012; Hsieh and Huang, 2004; Murphy, 2012). The small size of LDs provides a large surface area per unit TAG, which would facilitate lipase binding and lipolysis during germination (Huang and Huang, 2016). Recent proteomics and homology based studies have led to the identification of several new protein components involved in the formation, maintenance, and/or turnover of LDs (Pyc et al., 2017).

Regarding protein structural organization, oleosin comprises an N-terminal domain, a central hydrophobic domain, and a C-terminal domain (Hsiao and Tzen, 2011). Oleosin-H is distinguished from the other isoform oleosin-L by an extra 18-residue segment in its C-terminal domain (Tai et al., 2002). Ubiquitin is a highly-conserved regulatory protein that attaches to lysine ε-amino groups of target proteins by its C-terminal glycine residue (Hsiao and Tzen, 2011). Protein ubiquitination is integral to many biological pathways such as proteasomal degradation, stress responses, hormone biosynthesis and signaling, morphogenesis, chromatin structure, self-incompatibility, and battling pathogens (Sorokin et al., 2009). Some studies suggested that oleosin might be involved in storage lipid degradation after germination (Poxleitner et al., 2006). It has been noticed that protein ubiquitination is involved not only in the ubiquitin/26S proteasome pathway, but also in various biological functions possibly associated with different ubiquitin linkages (Weissman, 2001). Ectopic expression of several LD proteins, such as the plant oleosins and SEIPINs as well as the human perilipins, was shown to modulate LD morphology and accumulation in yeast (S. cerevisiae) (Cai et al., 2015). Lipid reserves are metabolized via the successive events of lipolysis, fatty acid (FA) transport to glyoxysomes, activation of acyl-CoA derivatives, β-oxidation, glyoxylate cycle, partial tricarboxylic acid cycle, and gluconeogenesis (Deruyffelaere et al., 2015).

In an embodiment, the oil body coating polypeptide is non-allergenic, or not known to be allergenic, such as to humans.

As used herein, the term “Oleosin” refers to an amphipathic protein present in the membrane of oil bodies in the storage tissues of seeds (see, for example, Huang, 1996; Tai et al., 2002; Lin et al., 2005; Capuano et al., 2007; Lui et al., 2009; Shimada and Hara-Nishimura, 2010) and artificially produced variants (see for example WO2011/053169 and WO2011/127118).

Oleosins are of low Mᵣ (15-26,000), corresponding to about 140-230 amino acid residues, which allows them to become tightly packed on the surface of oil bodies. Within each seed species, there are usually two or more oleosins of different Mᵣ. Each oleosin molecule contains a relatively hydrophilic, variable N-terminal domain (for example, about 48 amino acid residues), a central totally hydrophobic domain (for example, of about 70-80 amino acid residues) which is particularly rich in aliphatic amino acids such as alanine, glycine, leucine, isoleucine and valine, and an amphipathic «-helical domain of about 30-40 amino acid residues at or near the C-terminus. The central hydrophobic domain typically contains a proline knot motif of about 12 residues at its center. Generally, the central stretch of hydrophobic residues is inserted into the lipid core and the amphiphatic N-terminal and/or amphiphatic C-terminal are located at the surface of the oil bodies, with positively charged residues embedded in a phospholipid monolayer and the negatively charged ones exposed to the exterior.

As used herein, the term “Oleosin” encompasses polyoleosins which have multiple oleosin polypeptides fused together in a head-to-tail fashion as a single polypeptide (WO2007/045019), for example 2×, 4× or 6× oleosin peptides, and caleosins which bind calcium and which are a minor protein component of the proteins that coat oil bodies in seeds (Froissard et al., 2009), and steroleosins which bind sterols (WO2011/053169). However, generally a large proportion (at least 80%) of the oleosins of oil bodies will not be caleosins and/or steroleosins. The term “oleosin” also encompasses oleosin polypeptides which have been modified artificially, such oleosins which have one or more amino acid residues of the native oleosins artificially replaced with cysteine residues, as described in WO2011/053169. Typically, 4-8 residues are substituted artificially, preferably 6 residues, but as many as between 2 and 14 residues can be substituted. Preferably, both of the amphipathic N-terminal and C-terminal domains comprise cysteine substitutions. The modification increases the cross-linking ability of the oleosins and increases the thermal stability and/or the stability of the proteins against degradation by proteases.

A substantial number of oleosin protein sequences, and nucleotide sequences encoding therefor, are known from a large number of different plant species. Examples include, but are not limited to, oleosins from sesame, Arabidposis, canola, corn, rice, peanut, castor, soybean, flax, grape, cabbage, cotton, sunflower, sorghum and barley. Examples of oleosins (with their Accession Nos) include Brassica napus oleosin (CAA57545.1), Brassica napus oleosin S1-1 (ACG69504.1), Brassica napus oleosin S2-1 (ACG69503.1), Brassica napus oleosin S3-1 (ACG69513.1), Brassica napus oleosin S4-1 (ACG69507.1), Brassica napus oleosin S5-1 (ACG69511.1), Arachis hypogaea oleosin 1 (AAZ20276.1), Arachis hypogaea oleosin 2 (AAU21500.1), Arachis hypogaea oleosin 3 (AAU21501.1), Arachis hypogaea oleosin 5 (ABC96763.1), Ricinus communis oleosin 1 (EEF40948.1), Ricinus communis oleosin 2 (EEF51616.1), Glycine max oleosin isoform a (P29530.2), Glycine max oleosin isoform b (P29531.1), Linum usitatissimum oleosin low molecular weight isoform (ABB01622.1), Linum usitatissimum oleosin high molecular weight isoform (ABB01624.1), Helianthus annuus oleosin (CAA44224.1), Zea mays oleosin (NP_001105338.1), Brassica napus steroleosin (ABM30178.1), Brassica napus steroleosin SLO1-1 (ACG69522.1), Brassica napus steroleosin SLO2-1 (ACG69525.1), Sesamum indicum steroleosin (AAL13315.1), Sesame indicum OleosinL (Tai et al., 2002; Accession number AF091840; SEQ ID NO:86), Ficus pumila var. awkeotsang oleosin L-isoform (Accession No. ABQ57397.1), Cucumis sativus oleosinL (Accession No. XP_004146901.1), Linum usitatissimum oleosinL (Accession No. ABB01618.1), Glycine max oleosinL (Accession No. XP_003556321.2), Ananas comosus oleosinL (Accession No. OAY72596.1), Setaria italica oleosinL (Accession No. XP_004956407.1), Fragaria vesca subsp. vesca oleosinL (Accession No. XP_004307777.1), Brassica napus oleosinL (Accession No. CDY03377.1), Solanum lycopersicum oleosinL (Accession No. XP_004240765.1), Sesame indicum OleosinH1 (Tai et al., 2002; Accession number AF302807), Vanilla planifolia leaf OleosinU1 (Huang and Huang, 2016; Accession number SRX648194), Persea americana mesocarp OleosinM lipid droplet associated protein (Huang and Huang, 2016; Accession number SRX627420), Arachis hypogaea Oleosin 3 (Parthibane et al., 2012a and b; Accession number AY722696), A. thaliana Caleosin3 (Shen et al., 2014; Laibach et al., 2015; Accession number AK317039), A. thaliana steroleosin (Accession number AT081653), Zea mays steroleosin (NP_001152614.1), Brassica napus caleosin CLO-1 (ACG69529.1), Brassica napus caleosin CLO-3 (ACG69527.1), Sesamum indicum caleosin (AAF13743.1), Zea mays caleosin (NP_001151906.1), Glycine max caleosin (AAB71227). Other lipid encapsulation polypeptides that are functionally equivalent are plastoglobulins and MLDP polypeptides (WO2011/127118). In an embodiment, an exogenous polynucleotide of the invention which encodes a oleosin (such as an OleosinL) or steroleosin which comprises one or more of the following:
i) nucleotides encoding a polypeptide comprising amino acids whose sequence is set forth in any of the above mentioned accessions, or a biologically active fragment thereof, or a polypeptide whose amino acid sequence is at least 30% identical to that set forth in any of the above mentioned accessions,
ii) nucleotides whose sequence is at least 30% identical to i), and
iii) a polynucleotide which hybridizes to one or both of i) or ii) under stringent conditions.

In an embodiment, the oleosin is oleosinL or an ortholog thereof. OleosinL lacks the about 18 amino acid H-form insertion towards the C-terminus of other oleosins (see, for example, Tai et al., 2002). Thus, OleosinL's can readily be distinguished from other oleosins based on protein alignment.

As used herein, a “lipid droplet associated protein” or “LDAP” means a polypeptide which is associated with lipid droplets in plants in tissues or organs other than seeds, anthers and pollen, such as fruit tissues including pericarp and mesocarp. LDAPs may be associated with oil bodies in seeds, anthers or pollen as well as in the tissues or organs other than seeds, anthers and pollen. They are distinct from oleosins which are polypeptides associated with the surface of lipid droplets in seed tissues, anthers and pollen. LDAPs as used herein include LDAP polypeptides that are produced naturally in plant tissues as well as amino acid sequence variants that are produced artificially. The function of such variants can be tested as exemplified in Example 6.

Horn et al. (2013) identified two LDAP genes which are expressed in avocado pericarp. The encoded avocado LDAP1 and LDAP2 polypeptides were 62% identical in amino acid sequence and had homology to polypeptide encoded by Arabidopsis At3 g05500 and a rubber tree SRPP-like protein. Gidda et al. (2013) identified three LDAP genes that were expressed in oil palm (Elaeis guineensis) mesocarp but not in kernels and concluded that LDAP genes were plant specific and conserved amongst all plant species. LDAP polypeptides may contain additional domains (Gidda et al., (2013).

Genes encoding LDAPs are generally up-regulated in non-seed tissues with abundant lipid and can be identified thereby, but are thought to be expressed in all non-seed cells that produce oil including for transient storage. Horn et al. (2013) shows a phylogenetic tree of SRPP-like proteins in plants. Exemplary LDAP polypeptides are described in Example 6 and Example 9 herein, such as Rhodococcus opacus TadA lipid droplet associated protein (MacEachran et al., 2010; Accession number HM625859), Nannochloropsis oceanica LSDP oil body protein (Vieler et al., 2012; Accession number JQ268559) and Trichoderma reesei HFBI hydrophobin (Linder et al., 2005; Accession number Z68124). Homologs of LDAPs in other plant species can be readily identified by those skilled in the art. In an embodiment, an exogenous polynucleotide of the invention which encodes an LDAP which comprises one or more of the following:
i) nucleotides encoding a polypeptide comprising amino acids whose sequence is set forth in any of the above mentioned accessions, or a biologically active fragment thereof, or a polypeptide whose amino acid sequence is at least 30% identical to that set forth in any of the above mentioned accessions,
ii) nucleotides whose sequence is at least 30% identical to i), and
iii) a polynucleotide which hybridizes to one or both of i) or ii) under stringent conditions.

As used herein, the term a “polypeptide involved in starch biosynthesis” refers to any polypeptide, the downregulation of which in a plant cell below normal (wild-type) levels results in a reduction in the level of starch synthesis and a decrease in the levels of starch. This reduces the flow of carbon from sugars into starch. An example of such a polypeptide is AGPase.

As used herein, the term “ADP-glucose phosphorylase” or “AGPase” refers to an enzyme which regulates starch biosynthesis, catalysing conversion of glucose-1-phosphate and ATP to ADP-glucose which serves as the building block for starch polymers. The active form of the AGPase enzyme consists of 2 large and 2 small subunits.

The AGPase enzyme in plants exists primarily as a tetramer which consists of 2 large and 2 small subunits. Although these subunits differ in their catalytic and regulatory roles depending on the species (Kuhn et al., 2009), in plants the small subunit generally displays catalytic activity. The molecular weight of the small subunit is approximately 50-55 kDa. Sequences of exemplary AGPase small subunit polypeptides are provided in Accession Nos: XM_002462095.1 (Sorghum) and XM_008666513.1 (Zea mays) (Sanjaya et al., 2011; Zale et al., 2016). The molecular weight of the large subunit is approximately 55-60 kDa. The plant enzyme is strongly activated by 3-phosphoglycerate (PGA), a product of carbon dioxide fixation; in the absence of PGA, the enzyme exhibits only about 3% of its activity. Plant AGPase is also strongly inhibited by inorganic phosphate (Pi). In contrast, bacterial and algal AGPase exist as homotetramers of 50 kDa. The algal enzyme, like its plant counterpart, is activated by PGA and inhibited by Pi, whereas the bacterial enzyme is activated by fructose-1,6-bisphosphate (FBP) and inhibited by AMP and Pi.

### TAG Lipases and Beta-Oxidation

As used herein, the term “polypeptide involved in the degradation of lipid and/or which reduces lipid content” refers to any polypeptide which catabolises lipid, the downregulation of which in a plant cell below normal (wild-type) levels results an increase in the level of oil, such as fatty acids and/or TAGs, in a cell of a transgenic plant or part thereof such as a vegetative part, tuber, beet or a seed. Examples of such polypeptides include, but are not limited to, lipases, or a lipase such as a CGi58 (Comparative Gene identifier-58-Like) polypeptide, a SUGAR-DEPENDENT1 (SDP1) triacylglycerol lipase (see, for example, Kelly et al., 2011) and a lipase described in WO 2009/027335.

As used herein, the term “TAG lipase” (EC.3.1.1.3) refers to a protein which hydrolyzes TAG into one or more fatty acids and any one of DAG, MAG or glycerol. Thus, the term “TAG lipase activity” refers to the hydrolysis of TAG into glycerol and fatty acids.

As used herein, the term “CGi58” refers to a soluble acyl-CoA-dependent lysophosphatidic acid acyltransferase encoded by the At4 g24160 gene in Arabidopsis thaliana and its homologs in other plants and “Ict1p” in yeast and its homologs. The plant gene such as that from Arabidopsis gene locus At4 g24160 is expressed as two alternative transcripts: a longer full-length isoform (At4 g24160.1) and a smaller isoform (At4 g24160.2) missing a portion of the 3′ end (see James et al., 2010; Ghosh et al., 2009; US 201000221400). Both mRNAs code for a protein that is homologous to the human CGI-58 protein and other orthologous members of this a/B hydrolase family (ABHD). In an embodiment, the CGI58 (At4 g24160) protein contains three motifs that are conserved across plant species: a GXSXG lipase motif (SEQ ID NO:25), a HX(4)D acyltransferase motif (SEQ ID NO:26), and VX(3)HGF, a probable lipid binding motif (SEQ ID NO:27). The human CGI-58 protein has lysophosphatidic acid acyltransferase (LPAAT) activity but not lipase activity. In contrast, the plant and yeast proteins possess a canonical lipase sequence motif GXSXG (SEQ ID NO:25), that is absent from vertebrate (humans, mice, and zebrafish) proteins, and have lipase and phospholipase activity (Ghosh et al., 2009). Although the plant and yeast CGI58 proteins appear to possess detectable amounts of TAG lipase and phospholipase A activities in addition to LPAAT activity, the human protein does not.

Disruption of the homologous CGI-58 gene in Arabidopsis thaliana results in the accumulation of neutral lipid droplets in mature leaves. Mass spectroscopy of isolated lipid droplets from cgi-58 loss-of-function mutants showed they contain triacylglycerols with common leaf-specific fatty acids. Leaves of mature cgi-58 plants exhibit a marked increase in absolute triacylglycerol levels, more than 10-fold higher than in wild-type plants. Lipid levels in the oil-storing seeds of cgi-58 loss-of-function plants were unchanged, and unlike mutations in β-oxidation, the cgi-58 seeds germinated and grew normally, requiring no rescue with sucrose (James et al., 2010).

Examples of nucleotides encoding CGi58 polypeptides include those from Arabidopsis thaliana (NM_118548.1 encoding NP_194147.2), Brachypodium distachyon (XP_003578450.1), Glycine max (XM_003523590.1 encoding XP_003523638.1), Zea mays (NM_001155541.1 encoding NP_001149013.1), Sorghum bicolor (XM_002460493.1 encoding XP_002460538.1), Ricinus communis (XM_002510439.1 encoding XP_002510485.1), Medicago truncatula (XM_003603685.1 encoding XP_003603733.1), and Oryza sativa (encoding EAZ09782.1). In an embodiment, a genetic modification of the invention down-regulates endogenous production of CGi58, wherein CGi58 is encoded by one or more of the following:
i) nucleotides comprising a sequence set forth a the above mentioned accessions,
ii) nucleotides whose sequence is at least 30% identical to i), and
iii) a polynucleotide which hybridizes to one or both of i) or ii) under stringent conditions.

Other lipases which have lipase activity on TAG include SUGAR-DEPENDENT1 triacylglycerol lipase (SDP1, see for example Eastmond et al., 2006; Kelly et al., 2011) and SDP1-like polypeptides found in plant species as well as yeast (TGL4 polypeptide) and animal cells, which are involved in storage TAG breakdown. The SDP1 and SDP1-like polypeptides appear to be responsible for initiating TAG breakdown in seeds following germination (Eastmond et al., 2006). Plants that are mutant in SDP1, in the absence of exogenous WRI1 and DGAT1, exhibit increased levels of PUFA in their TAG. As used herein, “SDP1 polypeptides” include SDP1 polypeptides, SDP1-like polypeptides and their homologs in plant species. SDP1 and SDP1-like polypeptides in plants are 800-910 amino acid residues in length and have a patatin-like acylhydrolase domain that can associate with oil body surfaces and hydrolyse TAG in preference to DAG or MAG. SDP1 is thought to have a preference for hydrolysing the acyl group at the sn-2 position of TAG. Arabidopsis contains at least three genes encoding SDP1 lipases, namely SDP1 (Accession No. NP_196024, nucleotide sequence SEQ ID NO:37 and homologs in other species), SDP1L. (Accession No. NM_202720 and homologs in other species, Kelly et al., 2011) and ATGLL (At1 g33270) (Eastmond et al, 2006). Of particular interest for reducing gene activity are SDP1 genes which are expressed in vegetative tissues in plants, such as in leaves, stems and roots. Levels of non-polar lipids in vegetative plant parts can therefore be increased by reducing the activity of SDP1 polypeptides in the plant parts, for example by either mutation of an endogenous gene encoding a SDP1 polypeptide or introduction of an exogenous gene which encodes a silencing RNA molecule which reduces the expression of an endogenous SDP 1 gene. Such a reduction is of particular benefit in tuber crops such as sugarbeet and potato, and in “high sucrose” plants such as sweet sorghum, sugarcane and sugarbeet.

Genes encoding SDP1 homologues (including SDP1-like homologues) in a plant species of choice can be identified readily by homology to known SDP1 gene sequences. Known SDP1 nucleotide or amino acid sequences include Accession Nos.: in Brassica napus, GN078290, GN078281, GN078283; Capsella rubella, XP_006287072; Theobroma cacao, XP_007028574.1; Populus trichocarpa, XP_002308909; Prunus persica, XP_007203312; Prunus mume, XP 008240737; Malus domestica, XP_008373034; Ricinus communis, XP_002530081; Medicago truncatula, XP_003591425; Solanum lycopersicum, XP_004249208; Phaseolus vulgaris, XP_007162133; Glycine max, XP_003554141; Solanum tuberosum, XP_006351284; Glycine max, XP 003521151; Cicer arietinum, XP 004493431; Cucumis sativus, XP_004142709; Cucumis melo, XP 008457586; Jatropha curcas, KDP26217; Vitis vinifera, CBI30074; Oryza sativa, Japonica Group BAB61223; Oryza sativa, Indica Group EAY75912; Oryza sativa, Japonica Group NP_001044325; Sorghum bicolor, XP_002458531 (SEQ ID NO:38); Brachypodium distachyon, XP 003567139; Zea mays, AFW85009; Hordeum vulgare, BAK03290; Aegilops tauschii, EMT32802; Sorghum bicolor, XP_002463665; Zea mays, NP_001168677; Hordeum vulgare, BAK01155; Aegilops tauschii, EMT02623; Triticum urartu, EMS67257; Physcomitrella patens, XP 001758169. Preferred SDP1 sequences for use in genetic constructs for inhibiting expression of the endogenous genes are from cDNAs corresponding to the genes which are expressed most highly in the plant cells, vegetative plant parts or the seeds, whichever is to be modified. Nucleotide sequences which are highly conserved between cDNAs corresponding to all of the SDP 1 genes in a plant species are preferred if it is desired to reduce the activity of all members of a gene family in that species. In an embodiment, a genetic modification of the invention down-regulates endogenous production of SDP1, wherein SDP1 is encoded by one or more of the following:
i) nucleotides comprising a sequence set forth a the above mentioned accessions,
ii) nucleotides whose sequence is at least 30% identical to i), and
iii) a polynucleotide which hybridizes to one or both of i) or ii) under stringent conditions.

As shown in the Examples, reduction of the expression and/or activity of SDP1 TAG lipase in plant leaves greatly increased the TAG content, both in terms of the amount of TAG that accumulated and the earlier timing of accumulation during plant development, in the context of co-expression of the transcription factor WRI1 and a fatty acyl acyltransferase. In particular, the increase was observed in plants prior to flowering, and was up to about 70% on a weight basis (% dry weight) at the onset of senescence. The increase was relative to the TAG levels observed in corresponding plant leaves transformed with exogenous polynucleotides encoding the WRI1 and fatty acyl acyltransferase but lacking the modification that reduced SDP1 expression and/or activity.

Reducing the expression of other TAG catabolism genes in plant parts can also increase TAG content, such as the ACX genes encoding acyl-CoA oxidases such as the Acx1 (At4 g16760 and homologs in other plant species) or Acx2 (At5 g65110 and homologs in other plant species) genes. Another polypeptide involved in lipid catabolism is PXA1 which is a peroxisomal ATP-binding cassette transporter that is requires for fatty acid import for β-oxidation (Zolman et al. 2001).

Export of Fatty Acids from Plastids

As used herein, the term “polypeptide which increases the export of fatty acids out of plastids of the cell” refers to any polypeptide which aids in fatty acids being transferred from within plastids of plant cells in a plant or part thereof to outside the plastid, which may be any other part of the cell such as for example the endoplasmic reticulum (ER). Examples of such polypeptides include, but are not limited to, a C16 or C18 fatty acid thioesterase such as a FATA polypeptide or a FATB polypeptide, a C6 to C14 fatty acid thioesterase (which is also a FATB polypeptide), a fatty acid transporter such as an ABCA9 polypeptide or a long-chain acyl-CoA synthetase (LACS).

As used herein, the term “fatty acid thioesterase” or “FAT” or “acyl-ACP thioesterase” refers to an enzyme which catalyses the hydrolysis of the thioester bond between an acyl moiety and acyl carrier protein (ACP) in acyl-ACP and the release of a free fatty acid. Such enzymes typically function in the plastids of an organism which is synthesizing de novo fatty acids. As used herein, the term “C16 or C18 fatty acid thioesterase” refers to an enzyme which catalyses the hydrolysis of the thioester bond between a C16 and/or C18 acyl moiety and ACP in acyl-ACP and the release of free C16 or C18 fatty acid in the plastid. The free fatty acid is then re-esterified to CoA in the plastid envelope as it is transported out of the plastid. The substrate specificity of the fatty acid thioesterase (FAT) enzyme in the plastid is involved in determining the spectrum of chain length and degree of saturation of the fatty acids exported from the plastid. FAT enzymes can be classified into two classes based on their substrate specificity and nucleotide sequences, FATA and FATB (EC 3.1.2.14) (Jones et al., 1995). FATA polypeptides prefer oleoyl-ACP as substrate, while FATB polypeptides show higher activity towards saturated acyl-ACPs of different chain lengths such as acting on palmitoyl-ACP to produce free palmitic acid. Examples of FATA polypeptides useful for the invention include, but are not limited to, those from Arabidopsis thaliana (NP_189147), Arachis hypogaea (GU324446), Helianthus annuus (AAL79361), Carthamus tinctorius (AAA33020), Morus notabilis (XP_010104178.1), Brassica napus (CDX77369.1), Ricinus communis (XP_002532744.1) and Camelina sativa (AFQ60946.1). Examples of FATB polypeptides useful for the invention include, but are not limited to, those from Zea mays (AIL28766), Brassica napus (ABH11710), Helianthus annuus (AAX19387), Arabidopsis thaliana (AEE28300), Umbellularia californica (AAC49001), Arachis hypogaea (AFR54500), Ricinus communis (EEF47013) and Brachypodium sylvaticum (ABL85052.1). In an embodiment, an exogenous polynucleotide of the invention which encodes a thioesterase which comprises one or more of the following:
i) nucleotides encoding a polypeptide comprising amino acids whose sequence is set forth in any of the above mentioned accessions, or a biologically active fragment thereof, or a polypeptide whose amino acid sequence is at least 30% identical to that set forth in any of the above mentioned accessions,
ii) nucleotides whose sequence is at least 30% identical to i), and
iii) a polynucleotide which hybridizes to one or both of i) or ii) under stringent conditions.

A subclass of FATB polypeptides are fatty acid thioesterases which have hydrolysis activity on a C6C14 saturated acyl moiety linked by a thioester bond to ACP. Such enzymes are also referred to as medium chain fatty acid (MCFA) thioesterases or MC-FAT enzymes. Such enzymes may also have thioesterase activity on C16-ACP, indeed they may have greater thioesterase activity on a C16 acyl-ACP substrate than on a MCFA-ACP substrate, nevertheless they are considered herein to be an MCFA thioesterase if they produce at least 0.5% MCFA in the total fatty acid content when expressed exogenously in a plant cell. Examples of MCFA thioesterases are given in Example 10 herein. In a particularly preferred embodiment, the thioesterase has a preference for hydrolysing medium chain fatty acid substartes. For instance, the thioesterease comprises one or more of the following:
i) nucleotides encoding a polypeptide comprising amino acids whose sequence is set forth in any one of SEQ ID NOs 87 to 93, or a biologically active fragment thereof, or a polypeptide whose amino acid sequence is at least 30% identical to any one or more of both of SEQ ID NOs 87 to 93,
ii) nucleotides whose sequence is at least 30% identical to i), and
iii) a polynucleotide which hybridizes to one or both of i) or ii) under stringent conditions.

More particularly preferred embodiment, the thioesterease is a C12:0-ACP thioestersae which comprises one or more of the following:
i) nucleotides encoding a polypeptide comprising amino acids whose sequence is set forth in
SEQ ID NO:93, or a biologically active fragment thereof, or a polypeptide whose amino acid sequence is at least 30% identical to SEQ ID NO:93,
ii) nucleotides whose sequence is at least 30% identical to i), and
iii) a polynucleotide which hybridizes to one or both of i) or ii) under stringent conditions.

As used herein, the term “fatty acid transporter” relates to a polypeptide present in the plastid membrane which is involved in actively transferring fatty acids from a plastid to outside the plastid. Examples of ABCA9 (ABC transporter A family member 9) polypeptides useful for the invention include, but are not limited to, those from Arabidopsis thaliana (Q9FLT5), Capsella rubella (XP_006279962.1), Arabis alpine (KFK27923.1), Camelina sativa (XP_010457652.1), Brassica napus (CDY23040.1) and Brassica rapa (XP_009136512.1).

As used herein, the term “acyl-CoA synthetase” or “ACS” (EC 6.2.1.3) means a polypeptide which is a member of a ligase family that catalyzes the formation of fatty acyl-CoA by a two-step process proceeding through an adenylated intermediate, using a non-esterified fatty acid, CoA and ATP as substrates to produce an acyl-CoA ester, AMP and pyrophosphate as products. As used herein, the term “long-chain acyl-CoA synthetase” (LACS) is an ACS that has activity on at least a C18 free fatty acid substrate although it may have broader activity on any of C14-C20 free fatty acids. The endogenous plastidial LACS enzymes are localised in the outer membrane of the plastid and function with fatty acid thioesterase for the export of fatty acids from the plastid (Schnurr et al., 2002). In Arabidopsis, there are at least nine identified LACS genes (Shockey et al., 2002). Preferred LACS polypeptides are of the LACS9 subclass, which in Arabidopsis is the major plastidial LACS. Examples of LACS polypeptides useful for the invention include, but are not limited to, those from Arabidopsis thaliana (Q9CAP8), Camelina sativa (XP_010416710.1), Capsella rubella (XP_006301059.1), Brassica napus (CDX79212.1), Brassica rapa (XP_009104618.1), Gossypium raimondii (XP_012450538.1) and Vitis Vinifera (XP_002285853.1). Homologs of the above mentioned polypeptides in other species can readily be identified by those skilled in the art.

### Polypeptides Involved in Diacylglycerol (DAG) Production

Levels of non-polar lipids in, for example, vegetative plant parts can also be increased by reducing the activity of polypeptides involved in diacylglycerol (DAG) production in the plastid in the plant parts, for example by either mutation of an endogenous gene encoding such a polypeptide or introduction of an exogenous gene which encodes a silencing RNA molecule which reduces the expression of a target gene involved in diacylglycerol (DAG) production in the plastid.

As used herein, the term “polypeptide involved in diacylglycerol (DAG) production in the plastid” refers to any polypeptide in the plastid of plant cells in a plant or part thereof that is directly involved in the synthesis of diacylglycerol. Examples of such polypeptides include, but are not limited to, a plastidial GPAT, a plastidial LPAAT or a plastidial PAP.

GPATs are described elsewhere in the present document. Examples of plastidial GPAT polypeptides which can be targeted for down-regulation in the invention include, but are not limited to, those from Arabidopsis thaliana (BAA00575), Capsella rubella (XP_006306544.1), Camelina sativa (010499766.1), Brassica napus (CDY43010.1), Brassica rapa (XP_009145198.1), Helianthus annuus (ADV16382.1) and Citrus unshiu (BAB79529.1). Homologs in other species can readily be identified by those skilled in the art.

LPAATs are described elsewhere in the present document. As the skilled person would appreciate, plastidial LPAATs to be targeted for down-regulation for reducing DAG synthesis in the plastid are not endogenous LPAATs which function outside of the plastid such as those in the ER, for example being useful for producing TAG comprising medium chain length fatty acids. Examples of plastidial LPAAT polypeptides which can be targeted for down-regulation in the invention include, but are not limited to, those from Brassica napus (ABQ42862), Brassica rapa (XP_009137939.1), Arabidopsis thaliana (NP_194787.2), Camelina sativa (XP_010432969.1), Glycine max (XP_006592638.1) and Solanum tuberosum (XP_006343651.1). Homologs in other species of the above mentioned polypeptides can readily be identified by those skilled in the art.

As used herein, the term “phosphatidic acid phosphatase” (PAP) (EC 3.1.3.4) refers to a protein which hydrolyses the phosphate group on 3-sn-phosphatidate to produce 1,2-diacyl-sn-glycerol (DAG) and phosphate. Examples of plastidial PAP polypeptides which can be targeted for down-regulation in the invention include, but are not limited to, those from Arabidopsis thaliana (Q6NLA5), Capsella rubella (XP_006288605.1), Camelina sativa (XP_010452170.1), Brassica napus (CDY10405.1), Brassica rapa (XP_009122733.1), Glycine max (XP_003542504.1) and Solanum tuberosum (XP_006361792.1). Homologs in other species of the above mentioned polypeptides can readily be identified by those skilled in the art.

Another enzyme that results in DAG production, but in the ER rather than the plastid, is PDCT. As used herein, the term “phosphatidylcholine:diacylglycerol cholinephosphotransferase” (PDCT; EC 2.7.8.2) means an cholinephosphotransferase that transfers a phospho-choline headgroup from a phospholipid, typically PC, to produce DAG, or the reverse reaction to produce PC from DAG. Thus, the two substrates of the forward reaction are cytidine monophosphate (CMP) and phosphatidylcholine and the two products are CDP-choline and DAG. PDCT belongs to the phosphatidic acid phosphatase-related protein family and typically possesses lipid phosphatase/phosphotransferase (LPT) domains. In Arabidopsis thaliana, PDCT is encoded by the ROD1 (At3 g15820) and ROD2 (At3 g15830) genes (Lu et al., 2009). Homologous genes are readily identified in other plant species (Guan et al., 2015). Sequences of exemplary PDCT coding regions and polypeptides are provided in, Accession Nos XM 002437214 and EU973573.1), although any PDCT encoding gene can be used. In an embodiment, the PDCT is other than A. thaliana PDCT (Lu et al., 2009). Increased expression of PDCT, which may be exogenous or endogenous to the cell or plant of the invention and which is preferably expressed from an exogenous polynucleotide, increases the flow of esterified acyl groups from PC to DAG and thereby increases the TTQ in the total fatty acid content and the level of TAG in vegetative plant parts or cells of the invention. Alternatively, decreasing the level of PDCT activity in the cell or plant by mutation in the gene or by a silencing RNA molecule reduces the production of PC from DAG, the reverse PDCT reaction.

Import of Fatty Acids into Plastids

Levels of non-polar lipids in vegetative plant parts can also be increased by reducing the activity of TGD polypeptides in the plant parts, for example by either mutation of an endogenous gene encoding a TGD polypeptide or introduction of an exogenous gene which encodes a silencing RNA molecule which reduces the expression of an endogenous TGD gene. As used herein, a “Trigalactosyldiacylglycerol (TGD) polypeptide” is one which is involved in the ER to chloroplast lipid trafficking (Xu et al., 2010; Fan et al., 2015) and involved in forming a protein complex which has permease function for lipids. Four such polypeptides are known to form or be associated with a TGD permease, namely TGD-1 (Accession No. At1 g19800 and homologs in other species), TGD-2 (Accession No At2 g20320 and homologs in other species), TGD-3 (Accession No. NM-105215 and homologs in other species) and TGD-4 (At3 g06960 and homologs in other species) (US 20120237949). TGD5 is also involved in ER to choroplast lipid trafficking, and down-regulation of TGD5 is associated with increased oil production (US2015/337017; Fan et al., 2015). Sequences of exemplary TGD5 polypeptides are provided in Accession Nos XM_002442154 and EU972796.1). TGD-1, -2 and -3 polypeptides are thought to be components of an ATP-Binding Cassette (ABC) transporter associated with the inner envelope membrane of the chloroplast. TGD-2 and TGD-4 polypeptides bind to phosphatidic acid whereas TGD-3 polypeptide functions as an ATPase in the chloroplast stroma. As used herein, an “endogenous TGD gene” is a gene which encodes a TGD polypeptide in a plant. Mutations in TGD-1 gene in A. thaliana caused accumulation of triacylglycerols, oligogalactolipids and phosphatidic acid (PA) (Xu et al., 2005). Mutations in TGD genes or SDP1 genes, or indeed in any desired gene in a plant, can be introduced in a site-specific manner by artificial zinc finger nuclease (ZFN), TAL effector (TALEN) or CRISPR technologies (using a Cas9 type nuclease) as known in the art. Preferred exogenous genes encoding silencing RNAs are those encoding a double-stranded RNA molecule such as a hairpin RNA or an artificial microRNA precursor.

### Sucrose Metabolism

The TAG levels and/or the TTQ of the total fatty content in the cells, plants and plant parts of the invention can also be increased by modifying sucrose metabolism, particularly in the stems of plants such as sugarcane, Sorghum and Zea mays. In an embodiment, this is achieved by increasing expression of a sucrose metabolism polypeptide such as invertase or sucrose synthase, or of a sucrose transport polypeptide such as SUS1, SUS4, SUT2, SUT4, or SWEET. The effect of these polypeptides is to increase the supply of sucrose and its monosaccharide components in the cytosol of the cells and/or to decrease the transfer and/or storage of sucrose in the vacuoles of the cells, particularly in stem cells. Sequences of examples of these polypeptides are provided in SEQ ID NOs:274-292 of WO 2016/004473. Invertase such as bCIN, INV2 or INV3 acts to convert sucrose into hexoses which can be exported from the vacuoles into the cytoplasm (Mckinley et al., 2016). Increased expression of SUS1 or SUS4 breaks down cytosolic sucrose into hexoses for glycolysis and de novo fatty acid synthesis rather than transfer of the sucrose into vacuoles, such as in stem parenchyma cells (Mckinley et al., 2016). Increased expression of sugar transport polypeptides such as tonoplast sucrose exporter, for example SUT2 or SUT4, or SWEET polypeptide releases vacuolar sucrose for cytosolic glycolysis and increases de novo fatty acid biosynthesis (Bihmidine et al., 2016; Qazi et al., 2012; Schneider et al., 2012; Hedrich et al., 2015; Klemens et al., 2013).

The TAG levels and/or the TTQ of the total fatty content in the cells, plants and plant parts of the invention can also be increased by reducing the level of TST polypeptides such as TST1 or TST2, particularly in the stems of plants such as sugarcane, Sorghum and Zea mays. TST polypeptide can be decreased by mutation of the endogenous genes encoding the polypeptide, or by introduction of an exogenous polynucleotide that encodes a silencing RNA molecule. Sequences of exemplary TST cDNAs and polypeptides are provided as SEQ ID NOs:266-273 of WO 2016/004473.

### Fatty Acid Modifying Enzymes

As used herein, the term “FAD2” refers to a membrane bound delta-12 fatty acid desturase that desaturates oleic acid (C18:1 ^{Δ9}) to produce linoleic acid (C18:2^{Δ9,12}).

As used herein, the term “epoxygenase” or “fatty acid epoxygenase” refers to an enzyme that introduces an epoxy group into a fatty acid resulting in the production of an epoxy fatty acid. In preferred embodiment, the epoxy group is introduced at the 12th carbon on a fatty acid chain, in which case the epoxygenase is a Δ12-epoxygenase, especially of a C16 or C18 fatty acid chain. The epoxygenase may be a Δ9-epoxygenase, a Δ15 epoxygenase, or act at a different position in the acyl chain as known in the art. The epoxygenase may be of the P450 class. Preferred epoxygenases are of the mono-oxygenase class as described in WO98/46762. Numerous epoxygenases or presumed epoxygenases have been cloned and are known in the art. Further examples of expoxygenases include proteins comprising an amino acid sequence provided in SEQ ID NO:21 of WO 2009/129582, polypeptides encoded by genes from Crepis paleastina (CAA76156, Lee et al., 1998), Stokesia laevis (AAR23815) (monooxygenase type), Euphorbia lagascae (AAL62063) (P450 type), human CYP2J2 (arachidonic acid epoxygenase, U37143); human CYPIA1 (arachidonic acid epoxygenase, K03191), as well as variants and/or mutants thereof.

As used herein, the term, “hydroxylase” or “fatty acid hydroxylase” refers to an enzyme that introduces a hydroxyl group into a fatty acid resulting in the production of a hydroxylated fatty acid. In a preferred embodiment, the hydroxyl group is introduced at the 2nd, 12th and/or 17th carbon on a C18 fatty acid chain. Preferably, the hydroxyl group is introduced at the 12th carbon, in which case the hydroxylase is a Δ12-hydroxylase. In another preferred embodiment, the hydroxyl group is introduced at the 15th carbon on a C16 fatty acid chain. Hydroxylases may also have enzyme activity as a fatty acid desaturase. Examples of genes encoding Δ12-hydroxylases include those from Ricinus communis (AAC9010, van de Loo 1995); Physaria lindheimeri, (ABQ01458, Dauk et al., 2007); Lesquerella fendleri, (AAC32755, Broun et al., 1998); Daucus carota, (AAK30206); fatty acid hydroxylases which hydroxylate the terminus of fatty acids, for example: A, thaliana CYP86A1 (P48422, fatty acid ω-hydroxylase); Vicia sativa CYP94A1 (P98188, fatty acid ω-hydroxylase); mouse CYP2E1 (X62595, lauric acid ω-1 hydroxylase); rat CYP4A1 (M57718, fatty acid ω-hydroxylase), as well as variants and/or mutants thereof.

As used herein, the term “conjugase” or “fatty acid conjugase” refers to an enzyme capable of forming a conjugated bond in the acyl chain of a fatty acid. Examples of conjugases include those encoded by genes from Calendula officinalis (AF343064, Qiu et al., 2001); Vernicia fordii (AAN87574, Dyer et al., 2002); Punica granatum (AY178446, Iwabuchi et al., 2003) and Trichosanthes kirilowii (AY178444, Iwabuchi et al., 2003); as well as variants and/or mutants thereof.

As used herein, the term “acetylenase” or “fatty acid acetylenase” refers to an enzyme that introduces a triple bond into a fatty acid resulting in the production of an acetylenic fatty acid. In a preferred embodiment, the triple bond is introduced at the 2nd, 6th, 12th and/or 17th carbon on a C18 fatty acid chain. Examples acetylenases include those from Helianthus annuus (AA038032, ABC59684), as well as as variants and/or mutants thereof.

Examples of such fatty acid modifying genes include proteins according to the following Accession Numbers which are grouped by putative function, and homologues from other species: Δ12-acetylenases ABC00769, CAA76158, AAO38036, AAO38032; 412 conjugases AAG42259, AAG42260, AAN87574; 412-desaturases P46313, ABS18716, AAS57577, AAL61825, AAF04093, AAF04094; 412 epoxygenases XP_001840127, CAA76156, AAR23815; 412-hydroxylases ACF37070, AAC32755, ABQ01458, AAC49010; and Δ12P450 enzymes such as AF406732.

### Silencing Suppressors

In an embodiment, a transgenic plant or part thereof of the invention may comprise a silencing suppressor.

As used herein, a “silencing suppressor” enhances transgene expression in a plant or part thereof of the invention. For example, the presence of the silencing suppressor results in higher levels of a polypeptide(s) produced an exogenous polynucleotide(s) in a plant or part thereof of the invention when compared to a corresponding plant or part thereof lacking the silencing suppressor. In an embodiment, the silencing suppressor preferentially binds a dsRNA molecule which is 21 base pairs in length relative to a dsRNA molecule of a different length. This is a feature of at least the p19 type of silencing suppressor, namely for p19 and its functional orthologs. In another embodiment, the silencing suppressor preferentially binds to a double-stranded RNA molecule which has overhanging 5′ ends relative to a corresponding double-stranded RNA molecule having blunt ends. This is a feature of the V2 type of silencing suppressor, namely for V2 and its functional orthologs. In an embodiment, the dsRNA molecule, or a processed RNA product thereof, comprises at least 19 consecutive nucleotides, preferably whose length is 19-24 nucleotides with 19-24 consecutive basepairs in the case of a double-stranded hairpin RNA molecule or processed RNA product, more preferably consisting of 20, 21, 22, 23 or 24 nucleotides in length, and preferably comprising a methylated nucleotide, which is at least 95% identical to the complement of the region of the target RNA, and wherein the region of the target RNA is i) within a 5′ untranslated region of the target RNA, ii) within a 5′ half of the target RNA, iii) within a protein-encoding open-reading frame of the target RNA, iv) within a 3′ half of the target RNA, or v) within a 3′ untranslated region of the target RNA.

Further details regarding silencing suppressors are well known in the art and described in WO 2013/096992 and WO 2013/096993.

### Polynucleotides

The terms “polynucleotide”, and “nucleic acid” are used interchangeably. They refer to a polymeric form of nucleotides of any length, either deoxyribonucleotides or ribonucleotides, or analogs thereof. A polynucleotide of the invention may be of genomic, cDNA, semisynthetic, or synthetic origin, double-stranded or single-stranded and by virtue of its origin or manipulation: (1) is not associated with all or a portion of a polynucleotide with which it is associated in nature, (2) is linked to a polynucleotide other than that to which it is linked in nature, or (3) does not occur in nature. The following are non-limiting examples of polynucleotides: coding or non-coding regions of a gene or gene fragment, exons, introns, messenger RNA (mRNA), transfer RNA (tRNA), ribosomal RNA (rRNA), ribozymes, cDNA, recombinant polynucleotides, plasmids, vectors, isolated DNA of any sequence, isolated RNA of any sequence, chimeric DNA of any sequence, nucleic acid probes, and primers. For in vitro use, a polynucleotide may comprise modified nucleotides such as by conjugation with a labeling component.

As used herein, an “isolated polynucleotide” refers to a polynucleotide which has been separated from the polynucleotide sequences with which it is associated or linked in its native state, or a non-naturally occurring polynucleotide.

As used herein, the term “gene” is to be taken in its broadest context and includes the deoxyribonucleotide sequences comprising the transcribed region and, if translated, the protein coding region, of a structural gene and including sequences located adjacent to the coding region on both the 5′ and 3′ ends for a distance of at least about 2 kb on either end and which are involved in expression of the gene. In this regard, the gene includes control signals such as promoters, enhancers, termination and/or polyadenylation signals that are naturally associated with a given gene, or heterologous control signals, in which case, the gene is referred to as a “chimeric gene”. The sequences which are located 5′ of the protein coding region and which are present on the mRNA are referred to as 5′ non-translated sequences. The sequences which are located 3′ or downstream of the protein coding region and which are present on the mRNA are referred to as 3′ non-translated sequences. The term “gene” encompasses both cDNA and genomic forms of a gene. A genomic form or clone of a gene contains the coding region which may be interrupted with non-coding sequences termed “introns”, “intervening regions”, or “intervening sequences.” Introns are segments of a gene which are transcribed into nuclear RNA (nRNA). Introns may contain regulatory elements such as enhancers. Introns are removed or “spliced out” from the nuclear or primary transcript; introns are therefore absent in the mRNA transcript. A gene which contains at least one intron may be subject to variable splicing, resulting in alternative mRNAs from a single transcribed gene and therefore polypeptide variants. A gene in its native state, or a chimeric gene may lack introns. The mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above.

As used herein, “chimeric DNA” refers to any DNA molecule that is not naturally found in nature; also referred to herein as a “DNA construct” or “genetic construct”. Typically, a chimeric DNA comprises regulatory and transcribed or protein coding sequences that are not naturally found together in nature. Accordingly, chimeric DNA may comprise regulatory sequences and coding sequences that are derived from different sources, or regulatory sequences and coding sequences derived from the same source, but arranged in a manner different than that found in nature. The open reading frame may or may not be linked to its natural upstream and downstream regulatory elements. The open reading frame may be incorporated into, for example, the plant genome, in a non-natural location, or in a replicon or vector where it is not naturally found such as a bacterial plasmid or a viral vector. The term “chimeric DNA” is not limited to DNA molecules which are replicable in a host, but includes DNA capable of being ligated into a replicon by, for example, specific adaptor sequences.

A “transgene” is a gene that has been introduced into the genome by a transformation procedure. The term includes a gene in a progeny plant or part thereof such as a vegetative plant part which was introducing into the genome of a progenitor cell thereof. Such progeny cells etc may be at least a 3^{rd} or 4^{th} generation progeny from the progenitor cell which was the primary transformed cell, or of the progenitor transgenic plant (referred to herein as a TO plant). Progeny may be produced by sexual reproduction or vegetatively such as, for example, from tubers in potatoes or ratoons in sugarcane. The term “genetically modified”, “genetic modification” and variations thereof, is a broader term that includes introducing a gene into a cell by transformation or transduction, mutating a gene in a cell and genetically altering or modulating the regulation of a gene in a cell, or the progeny of any cell modified as described above.

A “genomic region” as used herein refers to a position within the genome where a transgene, or group of transgenes (also referred to herein as a cluster), have been inserted into a cell, or predecessor thereof. Such regions only comprise nucleotides that have been incorporated by the intervention of man such as by methods described herein.

A “recombinant polynucleotide” of the invention refers to a nucleic acid molecule which has been constructed or modified by artificial recombinant methods. The recombinant polynucleotide may be present in a cell of a plant or part thereof in an altered amount or expressed at an altered rate (e.g., in the case of mRNA) compared to its native state. In one embodiment, the polynucleotide is introduced into a cell that does not naturally comprise the polynucleotide. Typically an exogenous DNA is used as a template for transcription of mRNA which is then translated into a continuous sequence of amino acid residues coding for a polypeptide of the invention within the transformed cell. In another embodiment, the polynucleotide is endogenous to the plant or part thereof and its expression is altered by recombinant means, for example, an exogenous control sequence is introduced upstream of an endogenous gene of interest to enable the transformed plant or part thereof to express the polypeptide encoded by the gene, or a deletion is created in a gene of interest by ZFN, Talen or CRISPR methods.

A recombinant polynucleotide of the invention includes polynucleotides which have not been separated from other components of the cell-based or cell-free expression system, in which it is present, and polynucleotides produced in said cell-based or cell-free systems which are subsequently purified away from at least some other components. The polynucleotide can be a contiguous stretch of nucleotides or comprise two or more contiguous stretches of nucleotides from different sources (naturally occurring and/or synthetic) joined to form a single polynucleotide. Typically, such chimeric polynucleotides comprise at least an open reading frame encoding a polypeptide of the invention operably linked to a promoter suitable of driving transcription of the open reading frame in a cell of interest.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

A polynucleotide of, or useful for, the present invention may selectively hybridise, under stringent conditions, to a polynucleotide defined herein. As used herein, stringent conditions are those that: (1) employ during hybridisation a denaturing agent such as formamide, for example, 50% (v/v) formamide with 0.1% (w/v) bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (2) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS, and/or (3) employ low ionic strength and high temperature for washing, for example, 0.015 M NaCl/0.0015 M sodium citrate/0.1% SDS at 50° C.

Polynucleotides of the invention may possess, when compared to naturally occurring molecules, one or more mutations which are deletions, insertions, or substitutions of nucleotide residues. Polynucleotides which have mutations relative to a reference sequence can be either naturally occurring (that is to say, isolated from a natural source) or synthetic (for example, by performing site-directed mutagenesis or DNA shuffling on the nucleic acid as described above).

### Polynucleotides for Reducing Expression of Genes

### RNA Interference

RNA interference (RNAi) is particularly useful for specifically reducing the expression of a gene, which results in reduced production of a particular protein if the gene encodes a protein. Although not wishing to be limited by theory, Waterhouse et al. (1998) have provided a model for the mechanism by which dsRNA (duplex RNA) can be used to reduce protein production. This technology relies on the presence of dsRNA molecules that contain a sequence that is essentially identical to the mRNA of the gene of interest or part thereof. Conveniently, the dsRNA can be produced from a single promoter in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules is well within the capacity of a person skilled in the art, particularly considering Waterhouse et al. (1998), Smith et al. (2000), WO 99/32619, WO 99/53050, WO 99/49029, and WO 01/34815.

In one example, a DNA is introduced that directs the synthesis of an at least partly double stranded RNA product(s) with homology to the target gene to be inactivated such as, for example, a SDP1, TGD, plastidial GPAT, plastidial LPAAT, plastidial PAP, AGPase gene. The DNA therefore comprises both sense and antisense sequences that, when transcribed into RNA, can hybridize to form the double stranded RNA region. In one embodiment of the invention, the sense and antisense sequences are separated by a spacer region that comprises an intron which, when transcribed into RNA, is spliced out. This arrangement has been shown to result in a higher efficiency of gene silencing (Smith et al., 2000). The double stranded region may comprise one or two RNA molecules, transcribed from either one DNA region or two. The presence of the double stranded molecule is thought to trigger a response from an endogenous system that destroys both the double stranded RNA and also the homologous RNA transcript from the target gene, efficiently reducing or eliminating the activity of the target gene.

The length of the sense and antisense sequences that hybridize should each be at least 19 contiguous nucleotides, preferably at least 50 contiguous nucleotides, more preferably at least 100 or at least 200 contiguous nucleotides. Generally, a sequence of 100-1000 nucleotides corresponding to a region of the target gene mRNA is used. The full-length sequence corresponding to the entire gene transcript may be used. The degree of identity of the sense sequence to the targeted transcript (and therefore also the identity of the antisense sequence to the complement of the target transcript) should be at least 85%, at least 90%, or 95-100%. The RNA molecule may of course comprise unrelated sequences which may function to stabilize the molecule. The RNA molecule may be expressed under the control of a RNA polymerase II or RNA polymerase III promoter. Examples of the latter include tRNA or snRNA promoters.

Preferred small interfering RNA (“siRNA”) molecules comprise a nucleotide sequence that is identical to about 19-25 contiguous nucleotides of the target mRNA. Preferably, the siRNA sequence commences with the dinucleotide AA, comprises a GC-content of about 30-70% (preferably, 30-60%, more preferably 40-60% and more preferably about 45%-55%), and does not have a high percentage identity to any nucleotide sequence other than the target in the genome of the organism in which it is to be introduced, for example, as determined by standard BLAST search.

microRNA

MicroRNAs (abbreviated miRNAs) are generally 19-25 nucleotides (commonly about 20-24 nucleotides in plants) non-coding RNA molecules that are derived from larger precursors that form imperfect stem-loop structures. miRNAs bind to complementary sequences on target messenger RNA transcripts (mRNAs), usually resulting in translational repression or target degradation and gene silencing. Artificial miRNAs (amiRNAs) can be designed based on natural miRNAs for reducing the expression of any gene of interest, as well known in the art.

In plant cells, miRNA precursor molecules are believed to be largely processed in the nucleus. The pri-miRNA (containing one or more local double-stranded or “hairpin” regions as well as the usual 5′ “cap” and polyadenylated tail of an mRNA) is processed to a shorter miRNA precursor molecule that also includes a stem-loop or fold-back structure and is termed the “pre-miRNA”. In plants, the pre-miRNAs are cleaved by distinct DICER-like (DCL) enzymes, yielding miRNA:miRNA*duplexes. Prior to transport out of the nucleus, these duplexes are methylated.

In the cytoplasm, the miRNA strand from the miRNA:miRNA duplex is selectively incorporated into an active RNA-induced silencing complex (RISC) for target recognition. The RISC-complexes contain a particular subset of Argonaute proteins that exert sequence-specific gene repression (see, for example, Millar and Waterhouse, 2005; Pasquinelli et al., 2005; Almeida and Allshire, 2005).

### Cosuppression

Genes can suppress the expression of related endogenous genes and/or transgenes already present in the genome, a phenomenon termed homology-dependent gene silencing. Most of the instances of homologydependent gene silencing fall into two classes—those that function at the level of transcription of the transgene, and those that operate post-transcriptionally.

Post-transcriptional homology-dependent gene silencing (i.e., cosuppression) describes the loss of expression of a transgene and related endogenous or viral genes in transgenic plants. Cosuppression often, but not always, occurs when transgene transcripts are abundant, and it is generally thought to be triggered at the level of mRNA processing, localization, and/or degradation. Several models exist to explain how cosuppression works (see in Taylor, 1997).

Cosuppression involves introducing an extra copy of a gene or a fragment thereof into a plant in the sense orientation with respect to a promoter for its expression. The size of the sense fragment, its correspondence to target gene regions, and its degree of sequence identity to the target gene can be determined by those skilled in the art. In some instances, the additional copy of the gene sequence interferes with the expression of the target plant gene. Reference is made to WO 97/20936 and EP 0465572 for methods of implementing co-suppression approaches.

### Recombinant Vectors

One embodiment of the present invention includes a recombinant vector, which comprises at least one polynucleotide defined herein and is capable of delivering the polynucleotide into a host cell. Recombinant vectors include expression vectors. Recombinant vectors contain heterologous polynucleotide sequences, that is, polynucleotide sequences that are not naturally found adjacent to a polynucleotide defined herein, that preferably, are derived from a different species. The vector can be either RNA or DNA, and typically is a viral vector, derived from a virus, or a plasmid. Plasmid vectors typically include additional nucleic acid sequences that provide for easy selection, amplification, and transformation of the expression cassette in prokaryotic cells, e.g., pUC-derived vectors, pGEM-derived vectors or binary vectors containing one or more T-DNA regions. Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of prokaryotic and eukaryotic (especially plant) cells.

“Operably linked” as used herein, refers to a functional relationship between two or more nucleic acid (e.g., DNA) segments. Typically, it refers to the functional relationship of a transcriptional regulatory element (promoter) to a transcribed sequence. For example, a promoter is operably linked to a coding sequence of a polynucleotide defined herein, if it stimulates or modulates the transcription of the coding sequence in an appropriate cell. Generally, promoter transcriptional regulatory elements that are operably linked to a transcribed sequence are physically contiguous to the transcribed sequence, i.e., they are cis-acting. However, some transcriptional regulatory elements such as enhancers need not be physically contiguous or located in close proximity to the coding sequences whose transcription they enhance.

When there are multiple promoters present, each promoter may independently be the same or different.

Recombinant vectors may also contain one or more signal peptide sequences to enable an expressed polypeptide defined herein to be retained in the endoplasmic reticulum (ER) in the cell, or transfer into a plastid, and/or contain fusion sequences which lead to the expression of nucleic acid molecules as fusion proteins. Examples of suitable signal segments include any signal segment capable of directing the secretion or localisation of a polypeptide defined herein.

To facilitate identification of transformants, the recombinant vector desirably comprises a selectable or screenable marker gene. By “marker gene” is meant a gene that imparts a distinct phenotype to cells expressing the marker gene and thus, allows such transformed cells to be distinguished from cells that do not have the marker. A selectable marker gene confers a trait for which one can “select” based on resistance to a selective agent (e.g., a herbicide, antibiotic). A screenable marker gene (or reporter gene) confers a trait that one can identify through observation or testing, that is, by “screening” (e.g., β-glucuronidase, luciferase, GFP or other enzyme activity not present in untransformed cells). Exemplary selectable markers for selection of plant transformants include, but are not limited to, a hyg gene which encodes hygromycin B resistance; a neomycin phosphotransferase (nptII) gene conferring resistance to kanamycin, paromomycin; a glutathione-S-transferase gene from rat liver conferring resistance to glutathione derived herbicides as for example, described in EP 256223; a glutamine synthetase gene conferring, upon overexpression, resistance to glutamine synthetase inhibitors such as phosphinothricin as for example, described in WO 87/05327; an acetyltransferase gene from Streptomyces viridochromogenes conferring resistance to the selective agent phosphinothricin as for example, described in EP 275957; a gene encoding a 5-enolshikimate-3-phosphate synthase (EPSPS) conferring tolerance to N-phosphonomethylglycine as for example, described by Hinchee et al. (1988); a bar gene conferring resistance against bialaphos as for example, described in WO91/02071; a nitrilase gene such as b×n from Klebsiella ozaende which confers resistance to bromoxynil (Stalker et al., 1988); a dihydrofolate reductase (DHFR) gene conferring resistance to methotrexate (Thillet et al., 1988); a mutant acetolactate synthase gene (ALS) which confers resistance to imidazolinone, sulfonylurea, or other ALS-inhibiting chemicals (EP 154,204); a mutated anthranilate synthase gene that confers resistance to 5-methyl tryptophan; or a dalapon dehalogenase gene that confers resistance to the herbicide.

Preferably, the recombinant vector is stably incorporated into the genome of the cell such as the plant cell. Accordingly, the recombinant vector may comprise appropriate elements which allow the vector to be incorporated into the genome, or into a chromosome of the cell.

### Expression Vector

As used herein, an “expression vector” is a DNA vector that is capable of transforming a host cell and of effecting expression of one or more specified polynucleotides. Expression vectors of the present invention contain regulatory sequences such as transcription control sequences, translation control sequences, origins of replication, and other regulatory sequences that are compatible with the host cell and that control the expression of polynucleotides of the present invention. In particular, expression vectors of the present invention include transcription control sequences. Transcription control sequences are sequences which control the initiation, elongation, and termination of transcription. Particularly important transcription control sequences are those which control transcription initiation such as promoter, enhancer, operator and repressor sequences. The choice of the regulatory sequences used depends on the target organism such as a plant and/or target organ or tissue of interest. Such regulatory sequences may be obtained from any eukaryotic organism such as plants or plant viruses, or may be chemically synthesized. A number of vectors suitable for stable transfection of plant cells or for the establishment of transgenic plants have been described in for example, Pouwels et al., Cloning Vectors: A Laboratory Manual, 1985, supp. 1987, Weissbach and Weissbach, Methods for Plant Molecular Biology, Academic Press, 1989, and Gelvin et al., Plant Molecular Biology Manual, Kluwer Academic Publishers, 1990. Typically, plant expression vectors include for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, a transcription termination site, and/or a polyadenylation signal.

A number of constitutive promoters that are active in plant cells have been described. Suitable promoters for constitutive expression in plants include, but are not limited to, the cauliflower mosaic virus (CaMV) 35S promoter, the Figwort mosaic virus (FMV) 35S, the light-inducible promoter from the small subunit (SSU) of the ribulose-1,5-bis-phosphate carboxylase, the rice cytosolic triosephosphate isomerase promoter, the adenine phosphoribosyltransferase promoter of Arabidopsis, the rice actin 1 gene promoter, the mannopine synthase and octopine synthase promoters, the Adh promoter, the sucrose synthase promoter, the R gene complex promoter, and the chlorophyll α/β binding protein gene promoter. These promoters have been used to create DNA vectors that have been expressed in plants, see for example, WO 84/02913. All of these promoters have been used to create various types of plant-expressible recombinant DNA vectors.

For the purpose of expression in source tissues of the plant such as the leaf, seed, root or stem, it is preferred that the promoters utilized in the present invention have relatively high expression in these specific tissues. For this purpose, one may choose from a number of promoters for genes with tissue- or cell-specific, or -enhanced expression. Examples of such promoters reported in the literature include, the chloroplast glutamine synthetase GS2 promoter from pea, the chloroplast fructose-1,6-biphosphatase promoter from wheat, the nuclear photosynthetic ST-LSI promoter from potato, the serine/threonine kinase promoter and the glucoamylase (CHS) promoter from Arabidopsis thaliana. Also reported to be active in photosynthetically active tissues are the ribulose-1,5-bisphosphate carboxylase promoter from eastern larch (Larix laricina), the promoter for the Cab gene, Cab6, from pine, the promoter for the Cab-1 gene from wheat, the promoter for the Cab-1 gene from spinach, the promoter for the Cab IR gene from rice, the pyruvate, orthophosphate dikinase (PPDK) promoter from Zea mays, the promoter for the tobacco Lhcb1*2 gene, the Arabidopsis thaliana Suc2 sucrose-H30 symporter promoter, and the promoter for the thylakoid membrane protein genes from spinach (PsaD, PsaF, PsaE, PC, FNR, AtpC, AtpD, Cab, RbcS). Other promoters for the chlorophyll α/β-binding proteins may also be utilized in the present invention such as the promoters for LhcB gene and PsbP gene from white mustard (Sinapis alba).

A variety of plant gene promoters that are regulated in response to environmental, hormonal, chemical, and/or developmental signals, also can be used for expression of RNA-binding protein genes in plant cells, including promoters regulated by (1) heat, (2) light (e.g., pea RbcS-3A promoter, maize RbcS promoter), (3) hormones such as abscisic acid, (4) wounding (e.g., WunI), or (5) chemicals such as methyl jasmonate, salicylic acid, steroid hormones, alcohol, Safeners (WO 97/06269), or it may also be advantageous to employ (6) organ-specific promoters.

As used herein, the term “plant storage organ specific promoter” refers to a promoter that preferentially, when compared to other plant tissues, directs gene transcription in a storage organ of a plant. For the purpose of expression in sink tissues of the plant such as the tuber of the potato plant, the fruit of tomato, or the seed of soybean, canola, cotton, Zea mays, wheat, rice, and barley, it is preferred that the promoters utilized in the present invention have relatively high expression in these specific tissues. The promoter for β-conglycinin or other seed-specific promoters such as the napin, zein, linin and phaseolin promoters, can be used. Root specific promoters may also be used. An example of such a promoter is the promoter for the acid chitinase gene. Expression in root tissue could also be accomplished by utilizing the root specific subdomains of the CaMV 35S promoter that have been identified.

In a particularly preferred embodiment, the promoter directs expression in tissues and organs in which lipid biosynthesis takes place. Such promoters may act in seed development at a suitable time for modifying lipid composition in seeds. Preferred promoters for seed-specific expression include: 1) promoters from genes encoding enzymes involved in lipid biosynthesis and accumulation in seeds such as desaturases and elongases, 2) promoters from genes encoding seed storage proteins, and 3) promoters from genes encoding enzymes involved in carbohydrate biosynthesis and accumulation in seeds. Seed specific promoters which are suitable are, the oilseed rape napin gene promoter (U.S. Pat. No. 5,608,152), the Vicia faba USP promoter (Baumlein et al., 1991), the Arabidopsis oleosin promoter (WO 98/45461), the Phaseolus vulgaris phaseolin promoter (U.S. Pat. No. 5,504,200), the Brassica Bce4 promoter (WO 91/13980), or the legumin B4 promoter (Baumlein et al., 1992), and promoters which lead to the seed-specific expression in monocots such as maize, barley, wheat, rye, rice and the like. Notable promoters which are suitable are the barley lpt2 or lpt1 gene promoter (WO 95/15389 and WO 95/23230), or the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene, the rye secalin gene). Other promoters include those described by Broun et al. (1998), Potenza et al. (2004), US 20070192902 and US 20030159173. In an embodiment, the seed specific promoter is preferentially expressed in defined parts of the seed such as the cotyledon(s) or the endosperm. Examples of cotyledon specific promoters include, but are not limited to, the FP1 promoter (Ellerstrom et al., 1996), the pea legumin promoter (Perrin et al., 2000), and the bean phytohemagglutnin promoter (Perrin et al., 2000). Examples of endosperm specific promoters include, but are not limited to, the maize zein-1 promoter (Chikwamba et al., 2003), the rice glutelin-1 promoter (Yang et al., 2003), the barley D-hordein promoter (Horvath et al., 2000) and wheat HMW glutenin promoters (Alvarez et al., 2000). In a further embodiment, the seed specific promoter is not expressed, or is only expressed at a low level, in the embryo and/or after the seed germinates.

In another embodiment, the plant storage organ specific promoter is a fruit specific promoter. Examples include, but are not limited to, the tomato polygalacturonase, E8 and Pds promoters, as well as the apple ACC oxidase promoter (for review, see Potenza et al., 2004). In a preferred embodiment, the promoter preferentially directs expression in the edible parts of the fruit, for example the pith of the fruit, relative to the skin of the fruit or the seeds within the fruit.

In an embodiment, the inducible promoter is the Aspergillus nidulans alc system. Examples of inducible expression systems which can be used instead of the Aspergillus nidulans alc system are described in a review by Padidam (2003) and Corrado and Karali (2009). In another embodiment, the inducible promoter is a safener inducible promoter such as, for example, the maize In2-1 or In2-2 promoter (Hershey and Stoner, 1991), the safener inducible promoter is the maize GST-27 promoter (Jepson et al., 1994), or the soybean GH2/4 promoter (Ulmasov et al., 1995).

In another embodiment, the inducible promoter is a senescence inducible promoter such as, for example, senescence-inducible promoter SAG (senescence associated gene) 12 and SAG 13 from Arabidopsis (Gan, 1995; Gan and Amasino, 1995) and LSC54 from Brassica napus (Buchanan-Wollaston, 1994). Such promoters show increased expression at about the onset of senescence of plant tissues, in particular the leaves.

For expression in vegetative tissue leaf-specific promoters, such as the ribulose biphosphate carboxylase (RBCS) promoters, can be used. For example, the tomato RBCS1, RBCS2 and RBCS3A genes are expressed in leaves and light grown seedlings (Meier et al., 1997). A ribulose bisphosphate carboxylase promoters expressed almost exclusively in mesophyll cells in leaf blades and leaf sheaths at high levels, described by Matsuoka et al. (1994), can be used. Another leaf-specific promoter is the light harvesting chlorophyll a/b binding protein gene promoter (see, Shiina et al., 1997). The Arabidopsis thaliana myb-related gene promoter (Atmyb5) described by Li et al. (1996), is leaf-specific. The Atmyb5 promoter is expressed in developing leaf trichomes, stipules, and epidermal cells on the margins of young rosette and cauline leaves, and in immature seeds. A leaf promoter identified in maize by Busk et al. (1997), can also be used.

In some instances, for example when LEC2 or BBM is recombinantly expressed, it may be desirable that the transgene is not expressed at high levels. An example of a promoter which can be used in such circumstances is a truncated napin A promoter which retains the seed-specific expression pattern but with a reduced expression level (Tan et al., 2011).

The 5′ non-translated leader sequence can be derived from the promoter selected to express the heterologous gene sequence of the polynucleotide of the present invention, or may be heterologous with respect to the coding region of the enzyme to be produced, and can be specifically modified if desired so as to increase translation of mRNA. For a review of optimizing expression of transgenes, see Koziel et al. (1996). The 5′ non-translated regions can also be obtained from plant viral RNAs (Tobacco mosaic virus, Tobacco etch virus, Maize dwarf mosaic virus, Alfalfa mosaic virus, among others) from suitable eukaryotic genes, plant genes (wheat and maize chlorophyll a/b binding protein gene leader), or from a synthetic gene sequence. The present invention is not limited to constructs wherein the non-translated region is derived from the 5′ non-translated sequence that accompanies the promoter sequence. The leader sequence could also be derived from an unrelated promoter or coding sequence. Leader sequences useful in context of the present invention comprise the maize Hsp70 leader (U.S. Pat. Nos. 5,362,865 and 5,859,347), and the TMV omega element.

The termination of transcription is accomplished by a 3′ non-translated DNA sequence operably linked in the expression vector to the polynucleotide of interest. The 3′ non-translated region of a recombinant DNA molecule contains a polyadenylation signal that functions in plants to cause the addition of adenylate nucleotides to the 3′ end of the RNA. The 3′ non-translated region can be obtained from various genes that are expressed in plant cells. The nopaline synthase 3′ untranslated region, the 3′ untranslated region from pea small subunit Rubisco gene, the 3′ untranslated region from soybean 7S seed storage protein gene are commonly used in this capacity. The 3′ transcribed, non-translated regions containing the polyadenylate signal of Agrobacterium tumor-inducing (Ti) plasmid genes are also suitable.

Recombinant DNA technologies can be used to improve expression of a transformed polynucleotide by manipulating, for example, the efficiency with which the resultant transcripts are translated by codon optimisation according to the host cell species or the deletion of sequences that destabilize transcripts, and the efficiency of post-translational modifications.

### Transfer Nucleic Acids

Transfer nucleic acids can be used to deliver an exogenous polynucleotide to a cell and comprise one, preferably two, border sequences and one or more polynucleotides of interest. The transfer nucleic acid may or may not encode a selectable marker. Preferably, the transfer nucleic acid forms part of a binary vector in a bacterium, where the binary vector further comprises elements which allow replication of the vector in the bacterium, selection, or maintenance of bacterial cells containing the binary vector. Upon transfer to a eukaryotic cell, the transfer nucleic acid component of the binary vector is capable of integration into the genome of the eukaryotic cell or, for transient expression experiments, merely of expression in the cell.

As used herein, the term “extrachromosomal transfer nucleic acid” refers to a nucleic acid molecule that is capable of being transferred from a bacterium such as Agrobacterium sp., to a plant cell such as a plant leaf cell. An extrachromosomal transfer nucleic acid is a genetic element that is well-known as an element capable of being transferred, with the subsequent integration of a nucleotide sequence contained within its borders into the genome of the recipient cell. In this respect, a transfer nucleic acid is flanked, typically, by two “border” sequences, although in some instances a single border at one end can be used and the second end of the transferred nucleic acid is generated randomly in the transfer process. A polynucleotide of interest is typically positioned between the left border-like sequence and the right border-like sequence of a transfer nucleic acid. The polynucleotide contained within the transfer nucleic acid may be operably linked to a variety of different promoter and terminator regulatory elements that facilitate its expression, that is, transcription and/or translation of the polynucleotide. Transfer DNAs (T-DNAs) from Agrobacterium sp. such as Agrobacterium tumefaciens or Agrobacterium rhizogenes, and man made variants/mutants thereof are probably the best characterized examples of transfer nucleic acids. An example is P-DNA (“plant-DNA”) which comprises T-DNA border-like sequences from plants.

As used herein, “T-DNA” refers to a T-DNA of an Agrobacterium tumefaciens Ti plasmid or from an Agrobacterium rhizogenes Ri plasmid, or variants thereof which function for transfer of DNA into plant cells. The T-DNA may comprise an entire T-DNA including both right and left border sequences, but need only comprise the minimal sequences required in cis for transfer, that is, the right T-DNA border sequence. The T-DNAs of the invention have inserted into them, anywhere between the right and left border sequences (if present), the polynucleotide of interest. The sequences encoding factors required in trans for transfer of the T-DNA into a plant cell such as vir genes, may be inserted into the T-DNA, or may be present on the same replicon as the T-DNA, or preferably are in trans on a compatible replicon in the Agrobacterium host. Such “binary vector systems” are well known in the art. As used herein, “P-DNA” refers to a transfer nucleic acid isolated from a plant genome, or man made variants/mutants thereof, and comprises at each end, or at only one end, a T-DNA border-like sequence.

As used herein, a “border” sequence of a transfer nucleic acid can be isolated from a selected organism such as a plant or bacterium, or be a man made variant/mutant thereof. The border sequence promotes and facilitates the transfer of the polynucleotide to which it is linked and may facilitate its integration in the recipient cell genome. In an embodiment, a border-sequence is between 10-80 bp in length. Border sequences from T-DNA from Agrobacterium sp. are well known in the art and include those described in Lacroix et al. (2008).

Whilst traditionally only Agrobacterium sp. have been used to transfer genes to plants cells, there are now a large number of systems which have been identified/developed which act in a similar manner to Agrobacterium sp. Several non-Agrobacterium species have recently been genetically modified to be competent for gene transfer (Chung et al., 2006; Broothaerts et al., 2005). These include Rhizobium sp. NGR234, Sinorhizobium meliloti and Mezorhizobium loti.

As used herein, the terms “transfection”, “transformation” and variations thereof are generally used interchangeably. “Transfected” or “transformed” cells may have been manipulated to introduce the polynucleotide(s) of interest, or may be progeny cells derived therefrom.

### Plants

The invention also provides a plant or part thereof comprising two or more exogenous polynucleotides and/or genetic modifications as described herein. The term “plant” when used as a noun refers to whole plants, whilst the term “part thereof” refers to plant organs (e.g., leaves, stems, roots, flowers, fruit), single cells (e.g., pollen), seed, seed parts such as an embryo, endosperm, scutellum or seed coat, plant tissue such as vascular tissue, plant cells and progeny of the same. As used herein, plant parts comprise plant cells.

As used herein, the terms “in a plant” and “in the plant” in the context of a modification to the plant means that the modification has occurred in at least one part of the plant, including where the modification has occurred throughout the plant, and does not exclude where the modification occurs in only one or more but not all parts of the plant. For example, a tissue-specific promoter is said to be expressed “in a plant”, even though it might be expressed only in certain parts of the plant. Analogously, “a transcription factor polypeptide that increases the expression of one or more glycolytic and/or fatty acid biosynthetic genes in the plant” means that the increased expression occurs in at least a part of the plant.

As used herein, the term “plant” is used in it broadest sense, including any organism in the Kingdom Plantae. It also includes red and brown algae as well as green algae. It includes, but is not limited to, any species of flowering plant, grass, crop or cereal (e.g., oilseed, maize, soybean), fodder or forage, fruit or vegetable plant, herb plant, woody plant or tree. It is not meant to limit a plant to any particular structure. It also refers to a unicellular plant (e.g., microalga). The term “part thereof” in reference to a plant refers to a plant cell and progeny of same, a plurality of plant cells, a structure that is present at any stage of a plant's development, or a plant tissue. Such structures include, but are not limited to, leaves, stems, flowers, fruits, nuts, roots, seed, seed coat, embryos. The term “plant tissue” includes differentiated and undifferentiated tissues of plants including those present in leaves, stems, flowers, fruits, nuts, roots, seed, for example, embryonic tissue, endosperm, dermal tissue (e.g., epidermis, periderm), vascular tissue (e.g., xylem, phloem), or ground tissue (comprising parenchyma, collenchyma, and/or sclerenchyma cells), as well as cells in culture (e.g., single cells, protoplasts, callus, embryos, etc.). Plant tissue may be in planta, in organ culture, tissue culture, or cell culture.

As used herein, the term “vegetative tissue” or “vegetative plant part” is any plant tissue, organ or part other than organs for sexual reproduction of plants. The organs for sexual reproduction of plants are specifically seed bearing organs, flowers, pollen, fruits and seeds. Vegetative tissues and parts include at least plant leaves, stems (including bolts and tillers but excluding the heads), tubers and roots, but excludes flowers, pollen, seed including the seed coat, embryo and endosperm, fruit including mesocarp tissue, seed-bearing pods and seed-bearing heads. In one embodiment, the vegetative part of the plant is an aerial plant part. In another or further embodiment, the vegetative plant part is a green part such as a leaf or stem.

A “transgenic plant” or variations thereof refers to a plant that contains a transgene not found in a wild-type plant of the same species, variety or cultivar. Transgenic plants as defined in the context of the present invention include plants and their progeny which have been genetically modified using recombinant techniques to cause production of at least one polypeptide defined herein in the desired plant or part thereof. Transgenic plant parts has a corresponding meaning. The plant and plant parts of the invention may comprise genetic modifications, for example gene mutations, and be considered as “non-transgenic” provided they lack transgenes.

The terms “seed” and “grain” are used interchangeably herein. “Grain” refers to mature grain such as harvested grain or grain which is still on a plant but ready for harvesting, but can also refer to grain after imbibition or germination, according to the context. Mature grain commonly has a moisture content of less than about 18%. In a preferred embodiment, the moisture content of the grain is at a level which is generally regarded as safe for storage, preferably between 5% and 15%, between 6% and 8%, between 8% and 10%, or between 10% and 15%. “Developing seed” as used herein refers to a seed prior to maturity, typically found in the reproductive structures of the plant after fertilisation or anthesis, but can also refer to such seeds prior to maturity which are isolated from a plant. Mature seed commonly has a moisture content of less than about 12%.

As used herein, the term “plant storage organ” refers to a part of a plant specialized to store energy in the form of for example, proteins, carbohydrates, lipid. Examples of plant storage organs are seed, fruit, tuberous roots, and tubers. A preferred plant storage organ of the invention is seed.

As used herein, the term “phenotypically normal” refers to a genetically modified plant or part thereof, for example a plant such as a tragsenic plant, or a storage organ such as a seed, tuber or fruit of the invention not having a significantly reduced ability to grow and reproduce when compared to an unmodified plant or part thereof. Preferably, the biomass, growth rate, germination rate, storage organ size, seed size and/or the number of viable seeds produced is not less than 90% of that of a plant lacking said genetic modifications or exogenous polynucleotides when grown under identical conditions. This term does not encompass features of the plant which may be different to the wild-type plant but which do not effect the usefulness of the plant for commercial purposes such as, for example, a ballerina phenotype of seedling leaves. In an embodiment, the genetically modified plant or part thereof which is phenotypically normal comprises a recombinant polynucleotide encoding a silencing suppressor operably linked to a plant storage organ specific promoter and has an ability to grow or reproduce which is essentially the same as a corresponding plant or part thereof not comprising said polynucleotide.

Plants go through a series of growing stages from sowing of a seed, germination and emergence of a seedling, through to flowering, seed setting, physiological maturity and ultimately senescence. These stages are well known and readily defined, for example for Sorghum plants as follows. Taking the day the seedling first emerges above the soil as day 0, the vegetative stage of growth is defined herein as from 10 days to initiation of flowering at about 60-70 days, and physiogical maturity is reached at about 100 days, depending on the environmental conditions. The vegetative stage includes the boot leaf stage from about 45 days until the first flowering. The boot leaf is the last leaf formed on the plant, from which the panicle (head) emerges. The “boot leaf stage” is defined as from when the boot leaf has fully emerged to initiation of flowering.

As used herein, the term “commencement of flowering” or “initiation of flowering” with respect to a plant refers to the time that the first flower on the plant opens, or the time of onset of anthesis.

As used herein, the term “seed set” with respect to a seed-bearing plant refers to the time when the first seed of the plant reaches maturity. This is typically observable by the colour of the seed or its moisture content, well known in the art.

As used herein, the term “mature” as it relates to a plant leaf means that it has reached full size but has not begun to show signs of ageing or death such as yellowing and/or sensensce. The skilled person can readily determine whether a leaf of a particular plant can be considered as mature.

As used herein, the term “senescence” with respect to a whole plant refers to the final stage of plant development which follows the completion of growth, usually after the plant reaches maximum aerial biomass or height. Senescence begins when the plant aerial biomass reaches its maximum and begins to decline in amount and generally ends with death of most of the plant tissues. It is during this stage that the plant mobilizes and recycles cellular components from leaves and other parts which accumulated during growth to other parts to complete its reproductive development. Senescence is a complex, regulated process which involves new or increased gene expression of some genes. Often, some plant parts are senescing while other parts of the same plant continue to grow. Therefore, with respect to a plant leaf or other green organ, the term “senescence” as used herein refers to the time when the amount of chlorophyll in the leaf or organ begins to decrease. Senescence is typically associated with dessication of the leaf or organ, mostly in the last stage of senescence. Senescence is usually observable by the change in colour of the leaf from green towards yellow and eventually to brown when fully dessicated. It is believed that cellular senescence underlies plant and organ senescence.

Plants provided by or contemplated for use in the practice of the present invention include both monocotyledons and dicotyledons. In preferred embodiments, the plants of the present invention are crop plants (for example, cereals and pulses, maize, wheat, potatoes, rice, sorghum, millet, cassava, barley) or legumes such as soybean, beans or peas. The plants may be grown for production of edible roots, tubers, leaves, stems, flowers or fruit. The plants may be vegetable plants whose vegetative parts are used as food. The plants of the invention may be: Acrocomia aculeata (macauba palm), Arabidopsis thaliana, Aracinis hypogaea (peanut), Astrocaryum murumuru (murumuru), Astrocaryum vulgare (tucumã), Attalea geraensis (Indaiá-rateiro), Attalea humilis (American oil palm), Attalea oleifera (andaiá), Attalea phalerata (uricuri), Attalea speciosa (babassu), Avena sativa (oats), Beta vulgaris (sugar beet), Brassica sp. such as Brassica carinata, Brassica juncea, Brassica napobrassica, Brassica napus (canola), Camelina sativa (false flax), Cannabis sativa (hemp), Carthamus tinctorius (safflower), Caryocar brasiliense (pequi), Cocos nucifera (Coconut), Crambe abyssinica (Abyssinian kale), Cucumis melo (melon), Elaeis guineensis (African palm), Glycine max (soybean), Gossypium hirsutum (cotton), Helianthus sp. such as Helianthus annuus (sunflower), Hordeum vulgare (barley), Jatropha curcas (physic nut), Joannesia princeps (arara nut-tree), Lemna sp. (duckweed) such as Lemna aequinoctialis, Lemna disperma, Lemna ecuadoriensis, Lemna gibba (swollen duckweed), Lemna japonica, Lemna minor, Lemna minuta, Lemna obscura, Lemna paucicostata, Lemna perpusilla, Lemna tenera, Lemna trisulca, Lemna turionifera, Lemna valdiviana, Lemna yungensis, Licania rigida (oiticica), Linum usitatissimum (flax), Lupinus angustifolius (lupin), Mauritia flexuosa (buriti palm), Maximiliana maripa (inaja palm), Miscanthus sp. such as Miscanthus x giganteus and Miscanthus sinensis, Nicotiana sp. (tabacco) such as Nicotiana tabacum or Nicotiana benthamiana, Oenocarpus bacaba (bacaba-do-azeite), Oenocarpus bataua (patauã), Oenocarpus distichus (bacaba-de-leque), Oryza sp. (rice) such as Oryza sativa and Oryza glaberrima, Panicum virgatum (switchgrass), Paraqueiba paraensis (mari), Persea amencana (avocado), Pongamia pinnata (Indian beech), Populus trichocarpa, Ricinus communis (castor), Saccharum sp. (sugarcane), Sesamum indicum (sesame), Solanum tuberosum (potato), Sorghum sp. such as Sorghum bicolor, Sorghum vulgare, Theobroma grandiforum (cupuassu), Trifolium sp., Trithrinax brasiliensis (Brazilian needle palm), Triticum sp. (wheat) such as Triticum aestivum, Zea mays (corn), alfalfa (Medicago sativa), rye (Secale cerale), sweet potato (Lopmoea batatus), cassava (Manihot esculenta), coffee (Cofea spp.), pineapple (Anana comosus), citrus tree (Citrus spp.), cocoa (Theobroma cacao), tea (Camellia senensis), banana (Musa spp.), avocado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia) and almond (Prunus amygdalus).

In an embodiment, the plant is not a Nicotiana sp.

Other preferred plants include C4 grasses such as, in addition to those mentioned above, Andropogon gerardi, Bouteloua curtipendula, B. gracilis, Buchloe dactyloides, Schizachyrium scoparium, Sorghastrum nutans, Sporobolus cryptandrus; C3 grasses such as Elymus canadensis, the legumes Lespedeza capitata and Petalostemum villosum, the forb Aster azureus; and woody plants such as Quercus ellipsoidalis and Q. macrocarpa. Other preferred plants include C3 grasses.

In a preferred embodiment, the plant is an angiosperm.

In an embodiment, the plant is an oilseed plant, preferably an oilseed crop plant. As used herein, an “oilseed plant” is a plant species used for the commercial production of lipid from the seeds of the plant. The oilseed plant may be, for example, oil-seed rape (such as canola), maize, sunflower, safflower, soybean, sorghum, flax (linseed) or sugar beet. Furthermore, the oilseed plant may be other Brassicas, cotton, peanut, poppy, rutabaga, mustard, castor bean, sesame, safflower, Jatropha curcas or nut producing plants. The plant may produce high levels of lipid in its fruit such as olive, oil palm or coconut. Horticultural plants to which the present invention may be applied are lettuce, endive, or vegetable Brassicas including cabbage, broccoli, or cauliflower. The present invention may be applied in tobacco, cucurbits, carrot, strawberry, tomato, or pepper.

In a preferred embodiment, the plant is a member of the family Fabaceae (or Leguminosae) such as alfalfa, clover, peas, lucerne, beans, lentils, lupins, mesquite, carob, soybeans, and peanuts, or a member of the family Poaceae such as corn, sorghum, wheat, barley and oats. In a particularly preferred embodiment, the plant is alfalfa, clover, corn or sorghum, each of which are particularly useful for forage or fodder for animals.

In a preferred embodiment, the transgenic plant is homozygous for each and every gene that has been introduced (transgene) so that its progeny do not segregate for the desired phenotype. The transgenic plant may also be heterozygous for the introduced transgene(s), preferably uniformly heterozygous for the transgene such as for example, in F1 progeny which have been grown from hybrid seed. Such plants may provide advantages such as hybrid vigour, well known in the art.

### Transformation of Plants

Transgenic plants can be produced using techniques known in the art, such as those generally described in Slater et al., Plant Biotechnology—The Genetic Manipulation of Plants, Oxford University Press (2003), and Christou and Klee, Handbook of Plant Biotechnology, John Wiley and Sons (2004).

As used herein, the terms “stably transforming”, “stably transformed” and variations thereof refer to the integration of the polynucleotide into the genome of the cell such that they are transferred to progeny cells during cell division without the need for positively selecting for their presence. Stable transformants, or progeny thereof, can be identified by any means known in the art such as Southern blots on chromosomal DNA, or in situ hybridization of genomic DNA, enabling their selection.

Agrobacterium-mediated transfer is a widely applicable system for introducing genes into plant cells because DNA can be introduced into cells in whole plant tissues, plant organs, or explants in tissue culture, for either transient expression, or for stable integration of the DNA in the plant cell genome. For example, floral-dip (in planta) methods may be used. The use of Agrobacterium-mediated plant integrating vectors to introduce DNA into plant cells is well known in the art. The region of DNA to be transferred is defined by the border sequences, and the intervening DNA (T-DNA) is usually inserted into the plant genome. It is the method of choice because of the facile and defined nature of the gene transfer.

Acceleration methods that may be used include for example, microprojectile bombardment and the like. One example of a method for delivering transforming nucleic acid molecules to plant cells is microprojectile bombardment. This method has been reviewed by Yang et al., Particle Bombardment Technology for Gene Transfer, Oxford Press, Oxford, England (1994). Non-biological particles (microprojectiles) that may be coated with nucleic acids and delivered into cells, for example of immature embryos, by a propelling force. Exemplary particles include those comprised of tungsten, gold, platinum, and the like.

In another method, plastids can be stably transformed. Methods disclosed for plastid transformation in higher plants include particle gun delivery of DNA containing a selectable marker and targeting of the DNA to the plastid genome through homologous recombination (U.S. Pat. Nos. 5,451,513, 5,545,818, 5,877,402, 5,932,479, and WO 99/05265). Other methods of cell transformation can also be used and include but are not limited to the introduction of DNA into plants by direct DNA transfer into pollen, by direct injection of DNA into reproductive organs of a plant, or by direct injection of DNA into the cells of immature embryos followed by the rehydration of desiccated embryos.

The regeneration, development, and cultivation of plants from single plant protoplast transformants or from various transformed explants is well known in the art (Weissbach et al., In: Methods for Plant Molecular Biology, Academic Press, San Diego, Calif., (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polynucleotide is cultivated using methods well known to one skilled in the art.

To confirm the presence of the transgenes in transgenic cells and plants, a polymerase chain reaction (PCR) amplification or Southern blot analysis can be performed using methods known to those skilled in the art. Expression products of the transgenes can be detected in any of a variety of ways, depending upon the nature of the product, and include Northern blot hybridisation, Western blot and enzyme assay. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics. Preferably, the vegetative plant parts are harvested at a time when the yield of non-polar lipids are at their highest. In one embodiment, the vegetative plant parts are harvested about at the time of flowering, or after flowering has initiated. Preferably, the plant parts are harvested at about the time senescence begins, usually indicated by yellowing and drying of leaves.

Transgenic plants formed using Agrobacterium or other transformation methods typically contain a single genetic locus on one chromosome. Such transgenic plants can be referred to as being hemizygous for the added gene(s). More preferred is a transgenic plant that is homozygous for the added gene(s), that is, a transgenic plant that contains two added genes, one gene at the same locus on each chromosome of a chromosome pair. A homozygous transgenic plant can be obtained by self-fertilizing a hemizygous transgenic plant, germinating some of the seed produced and analyzing the resulting plants for the gene of interest.

It is also to be understood that two different transgenic plants that contain two independently segregating exogenous genes or loci can also be crossed (mated) to produce offspring that contain both sets of genes or loci. Selfing of appropriate F1 progeny can produce plants that are homozygous for both of the exogenous genes or loci. Back-crossing to a parental plant and out-crossing with a non-transgenic plant are also contemplated, as is vegetative propagation. Similarly, a transgenic plant can be crossed with a second plant comprising a genetic modification such as a mutant gene and progeny containing both of the transgene and the genetic modification identified. Descriptions of other breeding methods that are commonly used for different traits and crops can be found in Fehr, In: Breeding Methods for Cultivar Development, Wilcox J. ed., American Society of Agronomy, Madison Wis. (1987).

### Tilling

In one embodiment, TILLING (Targeting Induced Local Lesions IN Genomes) can be used to produce plants in which endogenous genes comprise a mutation, for example genes encoding an SDP1 or TGD polypeptide, TST, a plastidial GPAT, plastidial LPAAT, phosphatidic acid phosphatase (PAP), or a combination of two or more thereof. In a first step, introduced mutations such as novel single base pair changes are induced in a population of plants by treating seeds (or pollen) with a chemical mutagen, and then advancing plants to a generation where mutations will be stably inherited. DNA is extracted, and seeds are stored from all members of the population to create a resource that can be accessed repeatedly over time. For a TILLING assay, heteroduplex methods using specific endonucleases can be used to detect single nucleotide polymorphisms (SNPs). Alternatively, Next Generation Sequencing of DNA from pools of mutagenised plants can be used to identify mutants in the gene of choice. Typically, a mutation frequency of one mutant per 1000 plants in the mutagenised population is achieved. Using this approach, many thousands of plants can be screened to identify any individual with a single base change as well as small insertions or deletions (1-30 bp) in any gene or specific region of the genome. TILLING is further described in Slade and Knauf (2005), and Henikoff et al. (2004).

In addition to allowing efficient detection of mutations, high-throughput TILLING technology is ideal for the detection of natural polymorphisms. Therefore, interrogating an unknown homologous DNA by heteroduplexing to a known sequence reveals the number and position of polymorphic sites. Both nucleotide changes and small insertions and deletions are identified, including at least some repeat number polymorphisms. This has been called Ecotilling (Comai et al., 2004).

### Genome Editing Using Site-Specific Nucleases

Genome editing uses engineered nucleases such as RNA guided DNA endonucleases or nucleases composed of sequence specific DNA binding domains fused to a non-specific DNA cleavage module. These engineered nucleases enable efficient and precise genetic modifications by inducing targeted DNA double stranded breaks that stimulate the cell's endogenous cellular DNA repair mechanisms to repair the induced break. Such mechanisms include, for example, error prone non-homologous end joining (NHEJ) and homology directed repair (HDR).

In the presence of donor plasmid with extended homology arms, HDR can lead to the introduction of single or multiple transgenes to correct or replace existing genes. In the absence of donor plasmid, NHEJ-mediated repair yields small insertion or deletion mutations of the target that cause gene disruption.

Engineered nucleases useful in the methods of the present invention include zinc finger nucleases (ZFNs), transcription activator-like (TAL) effector nucleases (TALEN) and CRISPR/Cas9 type nucleases, and related nucleases.

Typically nuclease encoded genes are delivered into cells by plasmid DNA, viral vectors or in vitro transcribed mRNA.

A zinc finger nuclease (ZFN) comprises a DNA-binding domain and a DNA-cleavage domain, wherein the DNA binding domain is comprised of at least one zinc finger and is operatively linked to a DNA-cleavage domain. The zinc finger DNA-binding domain is at the N-terminus of the protein and the DNA-cleavage domain is located at the C-terminus of said protein.

A ZFN must have at least one zinc finger. In a preferred embodiment, a ZFN would have at least three zinc fingers in order to have sufficient specificity to be useful for targeted genetic recombination in a host cell or organism. Typically, a ZFN having more than three zinc fingers would have progressively greater specificity with each additional zinc finger.

The zinc finger domain can be derived from any class or type of zinc finger. In a particular embodiment, the zinc finger domain comprises the Cis₂His₂ type of zinc finger that is very generally represented, for example, by the zinc finger transcription factors TFIIIA or Sp1. In a preferred embodiment, the zinc finger domain comprises three Cis₂His₂ type zinc fingers. The DNA recognition and/or the binding specificity of a ZFN can be altered in order to accomplish targeted genetic recombination at any chosen site in cellular DNA. Such modification can be accomplished using known molecular biology and/or chemical synthesis techniques. (see, for example, Bibikova et al., 2002).

The ZEN DNA-cleavage domain is derived from a class of non-specific DNA cleavage domains, for example the DNA-cleavage domain of a Type II restriction enzyme such as FokI (Kim et al., 1996). Other useful endonucleases may include, for example, HhaI, HindIII, Nod, BbvCI, EcoRI, Bg/I, and A/I.

A transcription activator-like (TAL) effector nuclease (TALEN) comprises a TAL effector DNA binding domain and an endonuclease domain.

TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Thus, target sites can be predicted for TAL effectors, and TAL effectors can be engineered and generated for the purpose of binding to particular nucleotide sequences.

Fused to the TAL effector-encoding nucleic acid sequences are sequences encoding a nuclease or a portion of a nuclease, typically a nonspecific cleavage domain from a type II restriction endonuclease such as FokI (Kim et al., 1996). Other useful endonucleases may include, for example, HhaI, HindIII, Nod, BbvCI, EcoRI, BglI, and AlwI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

A sequence-specific TALEN can recognize a particular sequence within a preselected target nucleotide sequence present in a cell. Thus, in some embodiments, a target nucleotide sequence can be scanned for nuclease recognition sites, and a particular nuclease can be selected based on the target sequence. In other cases, a TALEN can be engineered to target a particular cellular sequence.

### Genome Editing Using Programmable RNA-Guided DNA Endonucleases

Distinct from the site-specific nucleases described above, the clustered regulatory interspaced short palindromic repeats (CRISPR)/Cas system provides an alternative to ZFNs and TALENs for inducing targeted genetic alterations, via RNA-guided DNA cleavage.

CRISPR systems rely on CRISPR RNA (crRNA) and transactivating chimeric RNA (tracrRNA) for sequence-specific cleavage of DNA. Three types of CRISPR/Cas systems exist: in type II systems, Cas9 serves as an RNA-guided DNA endonuclease that cleaves DNA upon crRNA-tracrRNA target recognition. CRISPR RNA base pairs with tracrRNA to form a two-RNA structure that guides the Cas9 endonuclease to complementary DNA sites for cleavage.

The CRISPR system can be portable to plant cells by co-delivery of plasmids expressing the Cas endonuclease and the necessary crRNA components. The Cas endonuclease may be converted into a nickase to provide additional control over the mechanism of DNA repair (Cong et al., 2013).

CRISPRs are typically short partially palindromic sequences of 24-40 bp containing inner and terminal inverted repeats of up to 11 bp. Although isolated elements have been detected, they are generally arranged in clusters (up to about 20 or more per genome) of repeated units spaced by unique intervening 20-58 bp sequences. CRISPRs are generally homogenous within a given genome with most of them being identical. However, there are examples of heterogeneity in, for example, the Archaea (Mojica et al., 2000).

### Feedstuffs

The present invention includes compositions which can be used as feedstuffs. For purposes of the present invention, “feedstuffs” include any food or preparation for animal (including human) consumption and which serves to nourish or build up tissues or supply energy, and/or to maintain, restore or support adequate nutritional status or metabolic function. Feedstuffs of the invention include nutritional compositions for babies and/or young children.

As used herein, the term “animal” refers to any eukaryotic organism capable of ingesting plant derived material. In an embodiment, the animal is a ruminant animal (cattle, sheep, goats etc). Alternatively, the animal is a non-ruminant animal. In one embodiment, the animal is a mammal. In an embodiment, the animal is a human. In an embodiment, the animal is a livestock animal such, but not limited to, as cattle, goats, sheep, pigs, horses, poultry such as chickens and the like. In an embodiment, the cattle are diary cattle or beef cattle. In another embodiment, the animal is a fish, for instance fish bred using aquaculture including, but not limited to, salmon, trout, carp, bass, bream, turbot, sole, milkfish, grey mullet, grouper, flounder, sea bass, cod, haddock, Japanese flounder, catfish, char, whitefish, sturgeon, tench, roach, pike, pike-perch, yellowtail, tilapia, eel or tropical fish (such as the fresh, brackish, and salt water tropical fish). The animal may be a crustacean such as, but not limited to, krill, clams, shrimp (including prawns), crab, and lobster.

Feedstuffs of the invention may comprise for example, a plant or part thereof such as a vegetative plant part of the invention along with a suitable carrier(s). The term “carrier” is used in its broadest sense to encompass any component which may or may not have nutritional value. As the person skilled in the art will appreciate, the carrier must be suitable for use (or used in a sufficiently low concentration) in a feedstuff, such that it does not have deleterious effect on an organism which consumes the feedstuff. Feedstuffs may comprise plant parts which have been harvested and subsequently processed or treated, for example, by chopping, cutting, drying, pressing or pelleting the plant parts, into a form that is suitable for consumption by the animal, or altered by processes such as drying or fermentation to produce hay or silage.

The feedstuff of the present invention comprises a lipid and/or protein produced directly or indirectly by use of the methods, plants or parts thereof disclosed herein. The composition may either be in a solid or liquid form. Additionally, the composition may include edible macronutrients, vitamins, and/or minerals in amounts desired for a particular use. The amounts of these ingredients will vary depending on whether the composition is intended for use with normal individuals or for use with individuals having specialized needs such as individuals suffering from metabolic disorders and the like.

Examples of suitable carriers with nutritional value include, but are not limited to, macronutrients such as edible fats, carbohydrates and proteins. Examples of such edible fats include, but are not limited to, coconut oil, borage oil, fungal oil, black current oil, soy oil, and mono- and di-glycerides. Examples of such carbohydrates include, but are not limited to, glucose, edible lactose, and hydrolyzed starch. Additionally, examples of proteins which may be utilized in the nutritional composition of the invention include, but are not limited to, soy proteins, electrodialysed whey, electrodialysed skim milk, milk whey, or the hydrolysates of these proteins.

With respect to vitamins and minerals, the following may be added to the feedstuff compositions of the present invention, calcium, phosphorus, potassium, sodium, chloride, magnesium, manganese, iron, copper, zinc, selenium, iodine, and vitamins A, E, D, C, and the B complex. Other such vitamins and minerals may also be added.

A feedstuff composition of the present invention may also be added to food even when supplementation of the diet is not required. For example, the composition may be added to food of any type, including, but not limited to, margarine, butter, cheeses, milk, yogurt, chocolate, candy, snacks, salad oils, cooking oils, cooking fats, meats, fish and beverages.

Additionally, material produced in accordance with the present invention may also be used as animal food supplements to alter an animal's tissue or milk fatty acid composition to one more desirable for human or animal consumption, or to reduce methane production in ruminant animals. Furthermore, feedstuffs of the invention can be used in aquaculture to increase the levels of fatty acids and nutrition in fish for human or animal consumption.

Preferred feedstuffs of the invention are the plants, seed and other plant parts such as leaves, fruits and stems which may be used directly as food or feed for humans or other animals. For example, animals may graze directly on such plants grown in the field, or be fed more measured amounts in controlled feeding. The invention includes the use of such plants and plant parts as feed for increasing the polyunsaturated fatty acid levels in humans and other animals.

For consumption by non-human animals the feedstuff may be in any suitable form for such as, but not limited to, silage, hay or pasture growing in a field. In an embodiment, the feedstuff for non-human consumption is a leguminous plant, or part thereof, which is a member of the family Fabaceae family (or Leguminosae) such as alfalfa, clover, peas, lucerne, beans, lentils, lupins, mesquite, carob, soybeans, and peanuts.

In embodiment, the animal is in a feedlot and/or a shed.

In an embodiment, the plant or fraction thereof comprises at least about 5%, at least about 10%, at least about 50%, at least about 75%, at least about 90% or all of the feedstuff.

### Silage

As used herein, “silage” is a relatively high-moisture fodder which has been produced and stored in a process called ensilage and which is typically fed to cattle, sheep or other ruminants. During the storage time, carbohydrates, lipids and proteins in the plant material ferment, producing organic acids, or are broken down oxidatively, or both. The plant material upon harvest and the post-fermentation plant materials are both included in silage as the term is used herein. Silage is typically made from grass crops such as maize, sorghum, oats or other cereals, or from mixed pasture grasses and legumes such as alfalfa or clover, using the green, above-ground parts of the plants. Silage is made either by placing cut vegetation (usually the whole above-ground plant biomass which can include reproductive tissues) in a pit or silo or other means for storage, and compressing it down so as to leave as little air as possible with the plant material. Oxygen is excluded to some extent by covering it with a plastic sheet or by wrapping the plant material tightly within plastic film (baling) to reduce air inflow. Silage is made from plant material with a suitable moisture content, generally about 50% to 60% of the fresh weight, depending on the means of storage and the degree of compression used and the amount of water that will be lost in storage, but not exceeding 75%. For sorghum and corn, harvest begins when the whole-plant moisture is at a suitable level, ideally a few days before it is ripe. For pasture-type crops, the plants are mowed and allowed to wilt for a day or so until the moisture content drops to a suitable level. Ideally the crop is mowed when in full flower and deposited in the pit or silo on the day of its cutting. At harvesting, or after, the plant material is shredded or chopped by the harvester into pieces typically about 1-5 cm long. The plant material may be placed in large heaps on the ground and compressed to reduce the amount of air, then covered with plastic, or into a silo. Alternatively, the plant material may be baled in plastic wrapping to exclude air, which typically requires a lower moisture content of about 30-40%, but still too damp to be stored as dry hay.

The cut or chopped, stored plant material undergoes mostly anaerobic fermentation, which starts about 48 hours after the pit or silo is filled. The fermentation process converts sugars and other carbohydrates such as hemicellulose to organic acids, mostly acetic, propionic, lactic and butyric acids. Fermentation starts after the trapped oxygen is consumed and is essentially complete after about two weeks of storage, or may continue for longer periods. When the plant material is closely packed, the supply of oxygen is limited and the fermentation results in the decomposition of the carbohydrates, some lipids and proteins in the material into the organic acids. This product is named sour silage. If, on the other hand, the fodder is more loosely packed, the main reaction is oxidation which proceeds more rapidly and the temperature rises. If the mass is compressed when the temperature is 60-75C, the reaction ceases and sweet silage results. Fermentation may be aided by inoculation with specific microorganisms such as lactic acid bacteria to speed fermentation or improve the resulting silage, e.g. with Lactobacillus plantarum.

Bulk silage is commonly fed to dairy cattle, while baled silage tends to be used for beef cattle, sheep and horses. The advantages of silage as animal feed are several. During fermentation, the silage bacteria act on the cellulose and other carbohydrates in the forage to produce the organic fatty acids, thereby lowering the pH. This inhibits competing bacteria that might cause spoilage and the organic acids thereby act as natural preservatives, improve digestibility and palatability. This preservative action is particularly important during winter in temperate regions, when green forage is unavailable.

Silage can be produced using techniques known in the art such as those described in CN 101940272 CN 103461658 CN 101946853, CN 101946853, CN 104381743, U.S. Pat. Nos. 3,875,304 and 6,224,916. Pellets for animal feed can be produced using techniques known in the art such as those described in U.S. Pat. Nos. 3,035,920, 3,573,924 and 5,871,802.

### Plant Biomass

An increase in the total lipid content of plant biomass equates to greater energy content, making its use as a feed or forage or in the production of biofuel more economical.

The main components of naturally occurring plant biomass are carbohydrates (approximately 75%, dry weight) and lignin (approximately 25%), which can vary with plant type. The carbohydrates are mainly cellulose or hemicellulose fibers, which impart strength to the plant structure, and lignin, which holds the fibers together. Plant biomass typically has a low energy density as a result of both its physical form and moisture content. This also makes it inconvenient and inefficient for storage and transport without some kind of pre-processing. There are a range of processes available to convert it into a more convenient form including: 1) physical pre-processing (for example, grinding) or 2) conversion by thermal (for example, combustion, gasification, pyrolysis) or chemical (for example, anaerobic digestion, fermentation, composting, transesterification) processes. In this way, the biomass is converted into what can be described as a biomass fuel.

### Combustion

Combustion is the process by which flammable materials are allowed to burn in the presence of air or oxygen with the release of heat. The basic process is oxidation. Combustion is the simplest method by which biomass can be used for energy, and has been used to provide heat. This heat can itself be used in a number of ways: 1) space heating, 2) water (or other fluid) heating for central or district heating or process heat, 3) steam raising for electricity generation or motive force. When the flammable fuel material is a form of biomass the oxidation is of predominantly the carbon (C) and hydrogen (H) in the cellulose, hemicellulose, lignin, and other molecules present to form carbon dioxide (CO₂) and water (H2O). The plants of the invention provide improved fuel for combustion by virtue of the increased lipid content.

### Gasification

Gasification is a partial oxidation process whereby a carbon source such as plant biomass, is broken down into carbon monoxide (CO) and hydrogen (H₂), plus carbon dioxide (CO₂) and possibly hydrocarbon molecules such as methane (CH₄). If the gasification takes place at a relatively low temperature, such as 700° C. to 1000° C., the product gas will have a relatively high level of hydrocarbons compared to high temperature gasification. As a result it may be used directly, to be burned for heat or electricity generation via a steam turbine or, with suitable gas clean up, to run an internal combustion engine for electricity generation. The combustion chamber for a simple boiler may be close coupled with the gasifier, or the producer gas may be cleaned of longer chain hydrocarbons (tars), transported, stored and burned remotely. A gasification system may be closely integrated with a combined cycle gas turbine for electricity generation (IGCC—integrated gasification combined cycle). Higher temperature gasification (1200° C. to 1600° C.) leads to few hydrocarbons in the product gas, and a higher proportion of CO and H2. This is known as synthesis gas (syngas or biosyngas) as it can be used to synthesize longer chain hydrocarbons using techniques such as Fischer-Tropsch (FT) synthesis. If the ratio of H2 to CO is correct (2:1) FT synthesis can be used to convert syngas into high quality synthetic diesel biofuel which is compatible with conventional fossil diesel and diesel engines.

### Pyrolysis

As used herein, the term “pyrolysis” means a process that uses slow heating in the absence of oxygen to produce gaseous, oil and char products from biomass. Pyrolysis is a thermal or thermo-chemical conversion of lipid-based, particularly triglyceride-based, materials. The products of pyrolysis include gas, liquid and a sold char, with the proportions of each depending upon the parameters of the process. Lower temperatures (around 400° C.) tend to produce more solid char (slow pyrolysis), whereas somewhat higher temperatures (around 500° C.) produce a much higher proportion of liquid (bio-oil), provided the vapour residence time is kept down to around Is or less. Temperatures of about 275° C. to about 375° C. can be used to produce liquid bio-oil having a higher proportion of longer chain hydrocarbons. Pyrolysis involves direct thermal cracking of the lipids or a combination of thermal and catalytic cracking. At temperatures of about 400-500° C., cracking occurs, producing short chain hydrocarbons such as alkanes, alkenes, alkadienes, aromatics, olefins and carboxylic acid, as well as carbon monoxide and carbon dioxide.

Four main catalyst types can be used including transition metal catalysts, molecular sieve type catalysts, activated alumina and sodium carbonate (Maher and Bressler, 2007). Examples are given in U.S. Pat. No. 4,102,938. Alumina (Al₂O₃) activated by acid is an effective catalyst (U.S. Pat. No. 5,233,109). Molecular sieve catalysts are porous, highly crystalline structures that exhibit size selectivity, so that molecules of only certain sizes can pass through. These include zeolite catalysts such as ZSM-5 or HZSM-5 which are crystalline materials comprising AlO₄ and SiO₄ and other silica-alumina catalysts. The activity and selectivity of these catalysts depends on the acidity, pore size and pore shape, and typically operate at 300-500° C. Transition metal catalysts are described for example in U.S. Pat. No. 4,992,605. Sodium carbonate catalyst has been used in the pyrolysis of oils (Dandik and Aksoy, 1998).

As used herein, “hydrothermal processing”, “HTP”, also referred to as “thermal depolymerisation” is a form of pyrolysis which reacts the plant-derived matter, specifically the carbon-containing material in the plant-derived matter, with hydrogen to produce a bio-oil product comprised predominantly of paraffinic hydrocarbons along with other gases and solids. A significant advantage of HTP is that the vegetative plant material does not need to be dried before forming the composition for the conversion reaction, although the vegetative plant material can be dried beforehand to aid in transport or storage of the biomass. The biomass can be used directly as harvested from the field. The reactor is any vessel which can withstand the high temperature and pressure used and is resistant to corrosion. The solvent used in the HTP includes water or is entirely water, or may include some hydrocarbon compounds in the form of an oil. Generally, the solvent in HTP lacks added alcohols. The conversion reaction may occur in an oxidative, reductive or inert environment. “Oxidative” as used herein means in the presence of air, “reductive” means in the presence of a reducing agent, typically hydrogen gas or methane, for example 10-15% H2 with the remainder of the gas being N2, and “inert” means in the presence of an inert gas such as nitrogen or argon. The conversion reaction is preferably carried out under reductive conditions. The carbon-containing materials that are converted include cellulose, hemi-cellulose, lignin and proteins as well as lipids. The process uses a conversion temperature of between 270° C. and 400° C. and a pressure of between 70 and 350 bar, typically 300° C. to 350° C. and a pressure between 100-170bar. As a result of the process, organic vapours, pyrolysis gases and charcoal are produced. The organic vapours are condensed to produce the bio-oil. Recovery of the bio-oil may be achieved by cooling the reactor and reducing the pressure to atmospheric pressure, which allows bio-oil (organic) and water phases to develop and the bio-oil to be removed from the reactor.

The yield of the recovered bio-oil is calculated as a percentage of the dry weight of the input biomass on a dry weight basis. It is calculated according to the formula: weight of bio-oil x 100/dry weight of the vegetative plant parts. The weight of the bio-oil does not include the weight of any water or solids which may be present in a bio-oil mixture, which are readily removed by filtration or other known methods.

The bio-oil may then be separated into fractions by fractional distillation, with or without additional refining processes. Typically, the fractions that condense at these temperatures are termed: about 370° C., fuel oil; about 300° C., diesel oil; about 200° C., kerosene; about 150° C., gasoline (petrol). Heavier fractions may be cracked into lighter, more desirable fractions, well known in the art. Diesel fuel typically is comprised of C13-C22 hydrocarbon compounds.

### Transesterification

“Transesterification” as used herein is the conversion of lipids, principally triacylglycerols, into fatty acid methyl esters or ethyl esters by reaction with short chain alcohols such as methanol or ethanol, in the presence of a catalyst such as alkali or acid. Methanol is used more commonly due to low cost and availability, but ethanol, propanol or butanol or mixtures of the alcohols can also be used. The catalysts may be homogeneous catalysts, heterogeneous catalysts or enzymatic catalysts. Homogeneous catalysts include ferric sulphate followed by KOH. Heterogeneous catalysts include CaO, K₃PO₄, and WO₃/ZrO₂. Enzymatic catalysts include Novozyme 435 produced from Candida antarctica.

Transesterification can be carried out on extracted oil, or preferably directly in situ in the vegetative plant material. The vegetative plant parts may be dried and milled prior to being used to prepare the composition for the conversion reaction, but does not need to be. The advantage of direct conversion to fatty acid esters, preferably FAME, is that the conversion can use lower temperatures and pressures and still provide good yields of the product, for example, comprising at least 50% FAME by weight. The yield of recovered bio-oil by transesterification is calculated as for the HTP process.

### Production of Non-Polar Lipids

Techniques that are routinely practiced in the art can be used to extract, process, purify and analyze the lipids such as the TAG produced by plants or parts thereof of the instant invention. Such techniques are described and explained throughout the literature in sources such as, Fereidoon Shahidi, Current Protocols in Food Analytical Chemistry, John Wiley & Sons, Inc. (2001) D1.1.1-D1.1.11, and Perez-Vich et al. (1998).

Production of Oil from Vegetative Plant Parts or Seed

Typically, vegetative plant parts or plant seeds are cooked, pressed, and/or extracted to produce crude vegetative oil or seedoil, which is then degummed, refined, bleached, and deodorized. Generally, techniques for crushing seed are known in the art. For example, oilseeds can be tempered by spraying them with water to raise the moisture content to, for example, 8.5%, and flaked using a smooth roller with a gap setting of 0.23 to 0.27 mm. Depending on the type of seed, water may not be added prior to crushing. Application of heat deactivates enzymes, facilitates further cell rupturing, coalesces the lipid droplets, and agglomerates protein particles, all of which facilitate the extraction process. Vegetative plant parts can be similarly treated, depending on the moisture content.

In an embodiment, the majority of the vegetative oil or seedoil is released by passage through a screw press. Cakes (vegetative plant meal, seedmeal) expelled from the screw press may then be solvent extracted for example, with hexane, using a heat traced column, or not be solvent treated, in which case it may be more suitable as animal feed. Alternatively, crude vegetative oil or seedoil produced by the pressing operation can be passed through a settling tank with a slotted wire drainage top to remove the solids that are expressed with the vegetative oil or seedoil during the pressing operation. The clarified vegetative oil or seedoil can be passed through a plate and frame filter to remove any remaining fine solid particles. Once the solvent is stripped from the crude oil, the pressed and extracted portions are combined and subjected to normal lipid processing procedures (i.e., degumming, caustic refining, bleaching, and deodorization).

Extraction of the lipid from vegetative plant parts of the invention uses analogous methods to those known in the art for seedoil extraction. One way is physical extraction, which often does not use solvent extraction. Expeller pressed extraction is a common type, as are the screw press and ram press extraction methods. Mechanical extraction is typically less efficient than solvent extraction where an organic solvent (e.g., hexane) is mixed with at least the plant biomass, preferably after the biomass is dried and ground. The solvent dissolves the lipid in the biomass, which solution is then separated from the biomass by mechanical action (e.g., with the pressing processes above). This separation step can also be performed by filtration (e.g., with a filter press or similar device) or centrifugation etc. The organic solvent can then be separated from the non-polar lipid (e.g., by distillation). This second separation step yields non-polar lipid from the plant and can yield a re-usable solvent if one employs conventional vapor recovery. In an embodiment, the oil and/or protein content of the plant part or seed is analysed by near-infrared reflectance spectroscopy as described in Hom et al. (2007) prior to extraction.

If the vegetative plant parts are not to be used immediately to extract the lipid it is preferably processed to ensure the lipid content is retained as much as possible (see, for example, Christie, 1993), such as by drying the vegetative plant parts.

### Degumming

Degumming is an early step in the refining of oils and its primary purpose is the removal of most of the phospholipids from the oil, which may be present as approximately 1-2% of the total extracted lipid. Addition of ˜2% of water, typically containing phosphoric acid, at 70-80° C. to the crude oil results in the separation of most of the phospholipids accompanied by trace metals and pigments. The insoluble material that is removed is mainly a mixture of phospholipids and triacylglycerols and is also known as lecithin. Degumming can be performed by addition of concentrated phosphoric acid to the crude oil to convert non-hydratable phosphatides to a hydratable form, and to chelate minor metals that are present. Gum is separated from the oil by centrifugation. The oil can be refined by addition of a sufficient amount of a sodium hydroxide solution to titrate all of the fatty acids and removing the soaps thus formed.

### Alkali Refining

Alkali refining is one of the refining processes for treating crude oil, sometimes also referred to as neutralization. It usually follows degumming and precedes bleaching. Following degumming, the oil can treated by the addition of a sufficient amount of an alkali solution to titrate all of the fatty acids and phosphoric acids, and removing the soaps thus formed. Suitable alkaline materials include sodium hydroxide, potassium hydroxide, sodium carbonate, lithium hydroxide, calcium hydroxide, calcium carbonate and ammonium hydroxide. This process is typically carried out at room temperature and removes the free fatty acid fraction. Soap is removed by centrifugation or by extraction into a solvent for the soap, and the neutralised oil is washed with water. If required, any excess alkali in the oil may be neutralized with a suitable acid such as hydrochloric acid or sulphuric acid.

### Bleaching

Bleaching is a refining process in which oils are heated at 90-120° C. for 10-30 minutes in the presence of a bleaching earth (0.2-2.0%) and in the absence of oxygen by operating with nitrogen or steam or in a vacuum. This step in oil processing is designed to remove unwanted pigments (carotenoids, chlorophyll, gossypol etc), and the process also removes oxidation products, trace metals, sulphur compounds and traces of soap.

### Deodorization

Deodorization is a treatment of oils and fats at a high temperature (200-260° C.) and low pressure (0.1-1 mm Hg). This is typically achieved by introducing steam into the oil at a rate of about 0.1 ml/minute/100 ml of oil. Deodorization can be performed by heating the oil to 260° C. under vacuum, and slowly introducing steam into the oil at a rate of about 0.1 ml/minute/100 ml of oil. After about 30 minutes of sparging, the oil is allowed to cool under vacuum. The oil is typically transferred to a glass container and flushed with argon before being stored under refrigeration. If the amount of oil is limited, the oil can be placed under vacuum for example, in a Parr reactor and heated to 260° C. for the same length of time that it would have been deodorized. This treatment improves the colour of the oil and removes a majority of the volatile substances or odorous compounds including any remaining free fatty acids, monoacylglycerols and oxidation products.

### Winterization

Winterization is a process sometimes used in commercial production of oils for the separation of oils and fats into solid (stearin) and liquid (olein) fractions by crystallization at sub-ambient temperatures. It was applied originally to cottonseed oil to produce a solid-free product. It is typically used to decrease the saturated fatty acid content of oils.

### Algae

Algae can produce 10 to 100 times as much mass as terrestrial plants in a year and can be cultured in open-ponds (such as raceway-type ponds and lakes) or in photobioreactors. The most common oil-producing algae can generally include the diatoms (bacillariophytes), green algae (chlorophytes), blue-green algae (cyanophytes), and golden-brown algae (chrysophytes). In addition a fifth group known as haptophytes may be used. Groups include brown algae and heterokonts. Specific non-limiting examples algae include the Classes: Chlorophyceae, Eustigmatophyceae, Prymnesiophyceae, Bacillariophyceae. Bacillariophytes capable of oil production include the genera Amphipleura, Amphora, Chaetoceros, Cyclotella, Cymbella, Fragilaria, Hantzschia, Navicula, Nitzschia, Phaeodactylum, and Thalassiosira. Specific non-limiting examples of chlorophytes capable of oil production include Ankistrodesmus, Botryococcus, Chlorella, Chlorococcum, Dunaliella, Monoraphidium, Oocystis, Scenedesmus, and Tetraselmis. In one aspect, the chlorophytes can be Chlorella or Dunaliella. Specific non-limiting examples of cyanophytes capable of oil production include Oscillatoria and Synechococcus. A specific example of chrysophytes capable of oil production includes Boekelovia. Specific non-limiting examples of haptophytes include Isochysis and Pleurochysis.

Specific algae useful in the present invention include, for example, Chlamydomonas sp. such as Chlamydomonas reinhardtii, Dunaliella sp. such as Dunaliella salina, Dunaliella tertiolecta, D. acidophila, D. Lateralis. D. martima. D. parva, D. polmorpha, D. primolecta, D. pseudosalina, D. quartolecta. D. viridis, Haematococcus sp., Chlorella sp. such as Chlorella vulgaris, Chlorella sorokiniana or Chlorella protothecoides, Thraustochytrium sp., Schizochytrium sp., Volvox sp, Nannochloropsis sp., Botryococcus braunii which can contain over 60 wt % lipid, Phaeodactylum tricornutum, Thalassiosira pseudonana, Isochrysis sp., Pavlova sp., Chlorococcum sp, Ellipsoidion sp., Neochloris sp., Scenedesmus sp.

Algae of the invention can be harvested using microscreens, by centrifugation, by flocculation (using for example, chitosan, alum and ferric chloride) and by froth flotation. Interrupting the carbon dioxide supply can cause algae to flocculate on its own, which is called “autoflocculation”. In froth flotation, the cultivator aerates the water into a froth, and then skims the algae from the top. Ultrasound and other harvesting methods are currently under development.

Lipid may be extracted from the algae by mechanical crushing. When algal mass is dried it retains its lipid content, which can then be “pressed” out with an oil press. Osmotic shock may also be used to release cellular components such as lipid from algae, and ultrasonic extraction can accelerate extraction processes. Chemical solvents (for example, hexane, benzene, petroleum ether) are often used in the extraction of lipids from algae. Enzymatic extraction using enzymes to degrade the cell walls may also be used to extract lipids from algae. Supercritical CO₂ can also be used as a solvent. In this method, CO₂ is liquefied under pressure and heated to the point that it becomes supercritical (having properties of both a liquid and a gas), allowing it to act as a solvent.

### Uses of Plant Lipids

The lipids produced by the methods described have a variety of uses. In some embodiments, the lipids are used as food oils. In other embodiments, the lipids are refined and used as lubricants or for other industrial uses such as the synthesis of plastics. In some preferred embodiments, the lipids are refined to produce biodiesel. Biodiesel can be made from oils derived from the plants, algae and fungi of the invention. Use of plant triacylglycerols for the production of biofuel is reviewed in Durrett et al. (2008). The resulting fuel is commonly referred to as biodiesel and has a dynamic viscosity range from 1.9 to 6.0 mm²s⁻¹ (ASTM D6751). Bioalcohol may produced from the fermentation of sugars or the biomass other than the lipid left over after lipid extraction. General methods for the production of biofuel can be found in, for example, Maher and Bressler (2007), Greenwell et al. (2010), Karmakar et al. (2010), Alonso et al. (2010), Liu et al. (2010). Gong and Jiang (2011), Endalew et al. (2011) and Semwal et al. (2011).

The present invention provides methods for increasing oil content in vegetative tissues. Plants of the present invention have increased energy content of leaves and/or stems such that the whole above-ground plant parts may be harvested and used to produce biofuel. Furthermore, the level of oleic acid is increased significantly while the polyunsaturated fatty acid alpha linolenic acid (ALA) was reduced. The plants, algae and fungi of the present invention thereby reduce the production costs of biofuel.

### Biodiesel

The production of biodiesel, or alkyl esters, is well known. There are three basic routes to ester production from lipids: 1) Base catalysed transesterification of the lipid with alcohol; 2) Direct acid catalysed esterification of the lipid with methanol; and 3) Conversion of the lipid to fatty acids, and then to alkyl esters with acid catalysis. Any method for preparing fatty acid alkyl esters and glyceryl ethers (in which one, two or three of the hydroxy groups on glycerol are etherified) can be used. For example, fatty acids can be prepared, for example, by hydrolyzing or saponifying TAG with acid or base catalysts, respectively, or using an enzyme such as a lipase or an esterase. Fatty acid alkyl esters can be prepared by reacting a fatty acid with an alcohol in the presence of an acid catalyst. Fatty acid alkyl esters can also be prepared by reacting TAG with an alcohol in the presence of an acid or base catalyst. Glycerol ethers can be prepared, for example, by reacting glycerol with an alkyl halide in the presence of base, or with an olefin or alcohol in the presence of an acid catalyst. The alkyl esters can be directly blended with diesel fuel, or washed with water or other aqueous solutions to remove various impurities, including the catalysts, before blending.

### Aviation Fuel

For improved performance of biofuels, thermal and catalytic chemical bond-breaking (cracking) technologies have been developed that enable converting bio-oils into bio-based alternatives to petroleum-derived diesel fuel and other fuels, such as jet fuel.

The use of medium chain fatty acid source, such produced by a cell of the invention, a plant or part thereof of the invention, a seed of of the invention, or a transgenic version of any one thereof, precludes the need for high-energy fatty acid chain cracking to achieve the shorter molecules needed for jet fuels and other fuels with low-temperature flow requirements. This method comprises cleaving one or more medium chain fatty acid groups from the glycerides to form glycerol and one or more free fatty acids. In addition, the method comprises separating the one or more medium chain fatty acids from the glycerol, and decarboxylating the one or more medium chain fatty acids to form one or more hydrocarbons for the production of the jet fuel.

### Compositions

The present invention also encompasses compositions, particularly pharmaceutical compositions, comprising one or more plants, plant parts, lipids, proteins, nitrogen containing molecules, or carbon containing molecules, produced using the methods of the invention.

A pharmaceutical composition may additionally comprise an active ingredient and a standard, well-known, non-toxic pharmaceutically-acceptable carrier, adjuvant or vehicle such as phosphate-buffered saline, water, ethanol, polyols, vegetable oils, a wetting agent, or an emulsion such as a water/oil emulsion. The composition may be in either a liquid or solid form. For example, the composition may be in the form of a tablet, capsule, ingestible liquid, powder, topical ointment or cream. Proper fluidity can be maintained for example, by the maintenance of the required particle size in the case of dispersions and by the use of surfactants. It may also be desirable to include isotonic agents for example, sugars, sodium chloride, and the like. Besides such inert diluents, the composition can also include adjuvants such as wetting agents, emulsifying and suspending agents, sweetening agents, flavoring agents and perfuming agents.

A typical dosage of a particular fatty acid is from 0.1 mg to 20 g, taken from one to five times per day (up to 100 g daily) and is preferably in the range of from about 10 mg to about 1, 2, 5, or 10 g daily (taken in one or multiple doses). As known in the art, a minimum of about 300 mg/day of fatty acid, especially polyunsaturated fatty acid, is desirable. However, it will be appreciated that any amount of fatty acid will be beneficial to the subject.

Possible routes of administration of the pharmaceutical compositions of the present invention include for example, enteral and parenteral. For example, a liquid preparation may be administered orally. Additionally, a homogenous mixture can be completely dispersed in water, admixed under sterile conditions with physiologically acceptable diluents, preservatives, buffers or propellants to form a spray or inhalant.

The dosage of the composition to be administered to the subject may be determined by one of ordinary skill in the art and depends upon various factors such as weight, age, overall health, past history, immune status, etc., of the subject.

Additionally, the compositions of the present invention may be utilized for cosmetic purposes. The compositions may be added to pre-existing cosmetic compositions, such that a mixture is formed, or a fatty acid produced according to the invention may be used as the sole “active” ingredient in a cosmetic composition.

### Polypeptides

The terms “polypeptide” and “protein” are generally used interchangeably herein.

A polypeptide or class of polypeptides may be defined by the extent of identity (% identity) of its amino acid sequence to a reference amino acid sequence, or by having a greater % identity to one reference amino acid sequence than to another. The % identity of a polypeptide to a reference amino acid sequence is typically determined by GAP analysis (Needleman and Wunsch, 1970; GCG program) with parameters of a gap creation penalty=5, and a gap extension penalty=0.3. The query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns two sequences over their entire length, and the extent of identity is determined over the full length of the reference sequence. The polypeptide or class of polypeptides may have the same enzymatic activity as, or a different activity than, or lack the activity of, the reference polypeptide. Preferably, the polypeptide has an enzymatic activity of at least 10% of the activity of the reference polypeptide.

As used herein a “biologically active fragment” is a portion of a polypeptide of the invention which maintains a defined activity of a full-length reference polypeptide for example, DGAT activity. Biologically active fragments as used herein exclude the full-length polypeptide. Biologically active fragments can be any size portion as long as they maintain the defined activity. Preferably, the biologically active fragment maintains at least 10% of the activity of the full length polypeptide.

With regard to a defined polypeptide or enzyme, it will be appreciated that % identity figures higher than those provided herein will encompass preferred embodiments. Thus, where applicable, in light of the minimum % identity figures, it is preferred that the polypeptide/enzyme comprises an amino acid sequence which is at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of the polypeptides defined herein can be prepared by introducing appropriate nucleotide changes into a nucleic acid defined herein, or by in vitro synthesis of the desired polypeptide. Such mutants include for example, deletions, insertions, or substitutions of residues within the amino acid sequence. A combination of deletions, insertions and substitutions can be made to arrive at the final construct, provided that the final polypeptide product possesses the desired characteristics.

Mutant (altered) polypeptides can be prepared using any technique known in the art, for example, using directed evolution or rathional design strategies (see below). Products derived from mutated/altered DNA can readily be screened using techniques described herein to determine if they possess transcription factor, fatty acid acyltransferase or OBC activities.

In designing amino acid sequence mutants, the location of the mutation site and the nature of the mutation will depend on characteristic(s) to be modified. The sites for mutation can be modified individually or in series for example, by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide removed and a different residue inserted in its place. The sites of greatest interest for substitutional mutagenesis to inactivate enzymes include sites identified as the active site(s). Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1 under the heading of “exemplary substitutions”.

TABLE 1
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

In a preferred embodiment a mutant/variant polypeptide has only, or not more than, one or two or three or four conservative amino acid changes when compared to a naturally occurring polypeptide. Details of conservative amino acid changes are provided in Table 1. As the skilled person would be aware, such minor changes can reasonably be predicted not to alter the activity of the polypeptide when expressed in a transgenic plant or part thereof. Mutants with desired activity may be engineered using standard procedures in the art such as by performing random mutagenesis, targeted mutagenesis, or saturation mutagenesis on known genes of interest, or by subjecting different genes to DNA shuffling.

### Examples

### Example 1. General Materials and Methods

### Expression of Genes in Plant Cells in a Transient Expression System

Genes were expressed in plant cells using a transient expression system essentially as described by Voinnet et al. (2003) and Wood et al. (2009). Binary vectors containing the coding region to be expressed by a strong constitutive e35S promoter containing a duplicated enhancer region were introduced into Agrobacterium tumefaciens strain AGL1. A chimeric binary vector, 35S:p19, for expression of the p19 viral silencing suppressor was separately introduced into AGL1, as described in WO2010/057246. A chimeric binary vector, 35S:V2, for expression of the V2 viral silencing suppressor was separately introduced into AGL1. The recombinant cells were grown to stationary phase at 28° C. in LB broth supplemented with 50 mg/L kanamycin and 50 mg/L rifampicin. The bacteria were then pelleted by centrifugation at 5000 g for 5 min at room temperature before being resuspended to OD600=1.0 in an infiltration buffer containing 10 mM MES pH 5.7, 10 mM MgCl₂ and 100 uM acetosyringone. The cells were then incubated at 28° C. with shaking for 3 hours after which the OD600 was measured and a volume of each culture, including the viral suppressor construct 35S:p19 or 35S:V2, required to reach a final concentration of OD600=0.125 added to a fresh tube. The final volume was made up with the above buffer. Leaves were then infiltrated with the culture mixture and the plants were typically grown for a further three to five days after infiltration before leaf discs were recovered for either purified cell lysate preparation or total lipid isolation.

Transformation of Sorghum bicolor L.

### Plant Material

Sorghum plants of the inbred cultivar TX-430 (Miller, 1984) were grown in a plant growth chamber (Conviron, PGC-20 flex) at 28±1° C. “day” temperature and 20±1° C. “night” temperature, with a 16 hr photoperiod at a light intensity during the “day” of 900-1000 LUx. Panicles were covered with white translucent paper bags before flowering. Immature embryos were harvested from panicles 12-15 days after anthesis. Panicles were washed several times with water and developing seeds that were uniform in size were isolated and surface-sterilized using 20% commercial bleach mixed with 0.1% Tween-20 for 15-20 min. They were then washed with sterile distilled water 3 times each for 20 min, and blotted dry in a laminar flow hood. Immature embryos (IEs) ranging from 1.4 to 2.5 mm in length were aseptically isolated in the laminar flow hood and used as the starting tissue for preparation of green regenerative tissue.

### Base Cultivation Media

Media used for plant transformation were based on MS (Murashige and Skoog, 1962), supplied by PhytoTechnology Laboratories (M519). The pH of the media was adjusted to 5.8 before sterilization at 121° C. for 15 min. Heat sensitive plant growth regulators and other additives such as Geneticin (G418, Sigma) used as a selection agent, were filter sterilized (0.2 μm) and added to the media after sterilization when the media had cooled to about 55° C. The optimized culture medium composition for the different stages of plant transformation from callus induction to plant regeneration from green tissue induced from immature embryos is presented in Table 2.

### Cultivation Methods and Materials

The isolated IEs ranging from 1.4 to 2.5 mm in length were placed onto callus induction media-osmotic medium (CIM-osmotic medium, Table 2) with their scutellum facing upward. The CIM base medium was modified to improve callus quality and induction frequency from immature embryos, as well as callus regeneration media, by including α-Lipoic acid (1 to 5 mg/l), Melatonin (5 to 10 mg/l) and 2-Aminoidan-2-phosphonic acid HCl (1 to 2 mg/l) unless otherwise stated. For the development of green tissue, immature embryos were incubated under fluorescent light of approximately 45-50 μmol s⁻¹ m⁻² (16 h/day) in a tissue culture room at 24±2° C. After three days of culture, the root and shoot poles of the immature embryos were aseptically separated and re-inoculated on to the same CIM and maintained under the same conditions as described above. They were subcultured every two weeks onto the same CIM for 6 weeks and evaluated for callus quality, callus induction efficiency and transformation efficiency.

TABLE 2
Media used in DEC tissue induction and transformation of sorghum

| Name of the | | | |
|---|---|---|---|
| medium | Composition | Culture duration | |
| CIM- | MS medium powder with vitamins, 4.33 g/l; 2,4-D, 1 mg/l; BAP, | 3-4 hrs before | |
| Osmotic | 0.5 mg/l; L-proline, 0.7 g/l; L-Lipoic acid, 1 mg/l; peptone, 0.82 | bombardment; | |
| Medium | g/l; Myo-inositol, 150 mg/l; Copper sulfate, 0.8 mg/l; Manitol, | o/n post | |
| | 36.4 g/l; Sorbitol, 36.4 g/l; Agar, 8.5 g/l, pH 5.8 | bombardment | |
| CIM-pre | MS medium powder with vitamins, 4.33 g/l; 2,4-D, 1 mg/l; BAP, | 3-4 | days |
| selection | 0.5 mg/l; L-proline, 0.7 g/l; L-Lipoic acid, 1 mg/l; peptone, 0.82 | | |
| medium | g/l; Myo-inosito, l 150 mg/l; Copper sulfate, 0.8 mg/l; Maltose, 30 | | |
| | g/l; L-cysteine, 50 mg/l; Ascorbic acid, 15 mg/l; Agar, 9 g/l, pH 5.8 | | |
| CIM-callus | MS medium powder with vitamins, 4.33 g/l; 2,4-D, 1 mg/l; BAP, | 4 | weeks |
| induction | 0.5 mg/l; L-proline, 0.7 g/l; L-Lipoic acid, 1 mg/l; peptone, 0.82 | | |
| medium/G25 | g/l; Myo-inositol, 150 mg/l; Copper sulfate, 0.8 mg/l; Maltose, 30 | | |
| | g/l; Geneticin, 25 mg/l; Agar, 9 g/l, pH 5.8 | | |
| SIM-shoot | MS medium powder with vitamins, 4.33 g/l; BAP, 1.0 mg/l; 2,4- | 2 | weeks |
| induction | D, 0.5 mg/l; L-proline, 0.7 g/l; L-Lipoic acid, 1 mg/l; peptone, 0.82 | | |
| medium/G25 | g/l; Myo-inositol, 150 mg/l; Copper sulfate, 0.8 mg/l; Maltose, 30 | | |
| | g/l; Geneticin, 25 mg/l; Agar, 9 g/l, pH 5.8 | | |
| SRM-shoot | MS medium powder with vitamins, 4.33 g/l; BAP, 1.0 mg/l; TDZ, | 2 | weeks |
| regeneration | 0.5 mg/l; L-proline, 0.7 g/l; L-Lipoic acid, 1 mg/l; peptone, 0.82 | | |
| medium/G25 | g/l; Myo-inositol, 150 mg/l; Copper sulfate, 0.8 mg/l; Maltose, 30 | | |
| | g/l; Geneticin, 25 mg/l; Agar, 9 g/l, pH 5.8 | | |
| SOG-shoot | MS medium powder with vitamins, 2.2 g/l; L-proline, 0.7 g/l; L- | 2 | weeks |
| out growth | Lipoic acid, 1 mg/l; peptone, 0.82 g/l; Myo-inositol, 150 mg/l; | | |
| medium/G30 | Copper sulfate, 0.8 mg/l; Sucrose, 15 g/l; Geneticin, 30 mg/l; | | |
| | Agar, 9 g/l, pH 5.8 | | |
| RIM-root | MS medium powder with vitamins, 4.33 g/l; L-proline, 0.7 g/l; L- | 4 | weeks |
| induction | Lipoic acid, 1 mg/l; peptone, 0.82 g/l; Myo-inositol, 150 mg/l; | | |
| medium/G15 | Copper sulfate, 0.8 mg/l; sucrose, 15 g/l; IAA, 1 mg/l; IBA, 1 mg/l; | | |
| | NAA, 1 mg/l; PVP, 2 g/l; Geneticin, 15 mg/l; Agar 9 g/l, pH 5.8 | | |

Callus initiated from IEs in the first 3-4 weeks on CIM were mostly embryogenic and slowly differentiated into embryogenic callus with nodular structures which were coloured from pale to darker green. Embryogenic calli with green nodular structures were selected and maintained on the same medium (CIM) by subculturing every 2 weeks for up to 6 months or more, for use as explants for transformation. This type of tissue is termed herein as “differentiating embryogenic callus” tissue or “DEC” tissue, since this tissue forms nodular structures of differentiating cells which maintain embryogenic and organogenic potential, even though the tissues were really a mixture of callus cells, cells forming nodular structures and granular structures, and intermediate cells which the inventors understood were on the developmental pathway somewhere between callus (which is undifferentiated cells) and the nodular structures. Sometimes, the tissues included early stage (globular) somatic embryos.

### Particle-Bombardment of Green Regenerative DEC Tissues

Plasmids containing a selectable marker gene encoding the neomycin phosphotransferase II (NptII) providing resistance to the antibiotic Geneticin, under the control of the pUbi promoter and terminated by the nos 3′ region, were made or obtained for experiments to achieve stable transformation or for co-bombardment with other plasmids. Plasmid DNAs were isolated using a Zymopure™ Maxiprep kit (USA) according to the manufacturer's instructions. As a control vector for transformation, a genetic vector was obtained which contained uidA (GUS) and bar genes designed for expression in plant cells. The uidA gene was under the regulatory control of a maize polyubiquitin promoter (pUbi) and an Agrobacterium tumefaciens octopine synthase polyadenylation/terminator (ocs 3′) sequence. The sequence between the promoter and the protein coding region included the 5′ UTR and first intron of the Ubi gene. The uidA reporter gene also contained, within its protein coding region, an intron from a castor bean catalase gene which prevented translation of functional GUS protein in Agrobacterium, thereby reducing the background GUS gene expression in inoculated plant tissues. Therefore, any GUS expression would be due to expression of the uidA gene in the plant cells. The bar gene was also under the regulatory control of a pUbi promoter and terminated with an Agrobacterium nopaline synthase 3′ regulatory sequence (nos 3′). The uidA/bar vector was initially used in experiments to detect transient gene expression in the sorghum DEC tissues.

Uniform healthy, green regenerative DEC tissues (4-5 mm in size), produced using methods described above and having been cultured for 6 weeks to 6 months from initiation, were used for microprojectile-mediated transformation (bombardment) with the plasmids. Approximately 15 uniform green DEC tissues (each 4-5 mm) were placed at the centre of a petri dish (90 mm diameter) containing CIM-osmotic medium (Table 2) and incubated in the dark for about 4 hrs prior to bombardment. Bombardment was performed with a PDS-1000 He device (Biorad, Hercules, CA) as described by Liu et al. (2014). Post bombardment, the tissues were kept on the same osmotic medium overnight and transferred to pre-selection medium the next morning

Green DEC tissues bombarded with the genetic vector plasmid having a selectable marker encoding NptII were transferred to CIM-PS medium for 3-4 days before any selection, with addition to the medium of two compounds as antioxidants, L-cysteine (50 mg/l) and ascorbic acid (15 mg/l) (Table 2). Without the addition of these antioxidants in pre-selection medium, many of the bombarded tissues turned brown, some quite dark brown in colour, and many lost any ability to grow further. After 3-4 days on pre-selection medium, some of the bombarded tissues were subjected to GUS staining and viewed under a microscope to count the distinctive blue (GUS positive) spots, to check that genes had been transferred and could be expressed. The inclusion of the two antioxidants in the pre-selection medium improved the efficiency of the transformation as shown by the transient expression of the GUS gene.

Selection and Regeneration of Transgenic Plants with Optimised Conditions

Following bombardment and 3-4 days culture on pre-selection medium without selective agent (Geneticin), the bombarded tissues had increased in size from 4-5 mm to about 6-7 mm. These tissues were transferred to selective medium CIM/G25 containing 25 mg/l Geneticin (Table 2) and cultured for a further 4 weeks. When possible, the bombarded tissues were split into 2-6 pieces each, increasing the recovery of independent transformants. All of the tissues were cultured on the media as described in Table 2 and maintained in order to regenerate putative transgenic plants.

Plants were regenerated efficiently upon growth on these media. Each bombarded tissue and the shoots obtained from it were subcultured and maintained separately for calculation of the transformation efficiency. Positive transformation was confirmed by PCR on plant genomic DNA isolated from shoot samples, showing the presence of the selectable marker gene. The number of transformants was calculated per input DEC tissue. Transformation efficiencies of about 50% were obtained, expressed as independent transformants per input bombarded tissue.

### Agrobacterium-Mediated Transformation of Green Regenerative DEC Tissues

Uniform healthy, green regenerative DEC tissues (4-5 mm in size) produced using methods described in the foregoing examples and which have been cultured for 6 weeks to 6 months from initiation, are used for Agrobacterium-mediated transformation.

Genetic vectors having T-DNA regions containing the genes for transformation were designed and made for transformation of green regenerative DEC tissues using Agrobacterium-mediated transformation. A control binary vector contained uidA (GUS) and bar genes designed for expression in plant cells. The uidA gene was under the regulatory control of a maize polyubiquitin promoter (pUbi) and an Agrobacterium tumefaciens octopine synthase polyadenylation/terminator (ocs 3′) sequence. The sequence between the promoter and the protein coding region included the 5′ UTR and first intron of the Ubi gene. The uidA reporter gene also contained, within its protein coding region, an intron from a castor bean catalase gene which prevented translation of functional GUS protein in Agrobacterium, thereby reducing the background GUS gene expression in inoculated plant tissues. Therefore, any GUS expression was due to expression of the uidA gene in the plant cells. The bar gene was also under the regulatory control of a pUbi promoter and terminated with an Agrobacterium nopaline synthase 3′ regulatory sequence (nos 3′).

A suitable Agrobacterium tumefaciens strain was obtained e.g., AGL1 as described in Lazo et al. (1991) and the genetic vector is introduced into the Agrobacterium tumefaciens strain by heat shock method.

Agrobacterium cultures harboring the genetic construct are grown in suitable medium e.g., LB medium, and under appropriate conditions to produce an Agrobacterium inoculum, after which time the uniform healthy, green regenerative DEC tissues are infected with Agrobacterium inoculum. The infected DEC tissues are blotted on sterile filter paper to remove excess Agrobacterium and transferred to co-cultivation medium, optionally supplemented with antioxidants, and incubated in the dark at approximately 22-24° C. for 2-4 days. Following incubation, the DEC tissues are treated with an appropriate agent to kill the Agrobacterium, washed in sterile water, transferred to an appropriate medium and allowed to grow. After 4-6 weeks, shoots are excised and cultured on shoot elongation medium, after which time putative transgenic shoots are then detected using appropriate assays.

Brassica napus Transformation

Brassica napus seeds were sterilized using chlorine gas as described by Kereszt et al. (2007) and germinated on tissue culture medium. Cotyledonary petioles with 2-4 mm stalk were isolated as described by Belide et al. (2013) and used as explants. A. tumefaciens AGL1 (Lazo et al., 1991) cultures containing the binary vector were prepared and cotyledonary petioles inoculated with the cultures as described by Belide et al. (2013). Infected cotyledonary petioles were cultured on MS medium supplemented with 1 mg/L TDZ+0.1 mg/L NAA+3 mg/L AgNO₃+250 mg/L cefotaxime, 50 mg/L timentin and 25 mg/L kanamycin and cultured for 4 weeks at 24° C. with 16 hr/8 hr light-dark photoperiod with a biweekly subculture on to the same medium. Explants with green callus were transferred to shoot initiation medium (MS+1 mg/L kinetin+3 mg/L AgNO₃+250 mg/L cefotaxime+50 mg/L timentin+25 mg/L kanamycin) and cultured for another 2-3 weeks. Small shoots (˜1 cm) were isolated from the resistant callus and transferred to shoot elongation medium (MS medium with 0.1 mg/L gibberelic acid+3 mg/L AgNO₃+250 mg/L cefotaxime+25 mg/L kanamycin) and cultured for another two weeks. Healthy shoots with one or two leaves were selected and transferred to rooting media (1/2 MS with 1 mg/L NAA+20 mg/L ADS+3 mg/L AgNO₃+250 mg/L cefotaxime) and cultured for 2-3 weeks. DNA was isolated from small leaves of resistant shoots using the plant DNA isolation kit (Bioline, Alexandria, NSW, Australia) according to the manufacturer's protocol. Presence of T-DNA sequences was tested by PCR ampl. on genomic DNA. Positive, transgenic shoots with roots were transferred to pots with seedling raising mix and grown in a glasshouse at 24° C. daytime/16° C. night-time (stnd. conditions).

### Purified Leaf Lysate—Enzyme Assays

Nicotiana benthamiana leaf tissues previously infiltrated as described above were ground in a solution containing 0.1 M potassium phosphate buffer (pH 7.2) and 0.33 M sucrose using a glass homogenizer. Leaf homogenate was centrifuged at 20,000 g for 45 minutes at 4° C. after which each supernatant was collected. Protein content in each supernatant was measured according to Bradford (1976) using a Wallac1420 multi-label counter and a Bio-Rad Protein Assay dye reagent (Bio-Rad Laboratories, Hercules, CA USA). Acyltransferase assays used 100 μg protein according to Cao et al. (2007) with some modifications. The reaction medium contained 100 mM Tris-HCl (pH 7.0), 5 mM MgCl₂, 1 mg/mL BSA (fatty acid-free), 200 mM sucrose, 40 mM cold oleoyl-CoA, 16.4 uM sn-2 monooleoylglycerol[¹⁴C] (55 mCi/mmol, American Radiochemicals, Saint Louis, MO USA) or 6.0 uM [¹⁴C]glycerol-3-phosphate (G-3-P) disodium salt (150 mCi/mmol, American Radiochemicals). The assays were carried out for 7.5, 15, or 30 minutes.

### Lipid Analysis

### Analysis of Oil Content in Seeds

When seed oil content or total fatty acid composition was to be determined in small seeds such as Arabidopsis seeds, fatty acids in the seeds were directly methylated without crushing of seeds. Seeds were dried in a desiccator for 24 hours and approximately 4 mg of seed was transferred to a 2 ml glass vial containing a Teflon-lined screw cap. 0.05 mg triheptadecanoin (TAG with three C17:0 fatty acids) dissolved in 0.1 ml toluene was added to the vial as internal standard. Seed fatty acids were methylated by adding 0.7 ml of IN methanolic HCl (Supelco) to the vial containing seed material. Crushing of the seeds was not necessary for complete methylation with small seeds such as Arabidopsis seeds. The mixture was vortexed briefly and incubated at 80° C. for 2 hours. After cooling the mixtures to room temperature, 0.3 ml of 0.9% NaCl (w/v) and 0.1 ml hexane was added to the vial and mixed well for 10 minutes in a Heidolph Vibramax 110. The FAME were collected into 0.3 ml glass insert and analysed by GC with flame ionization detector (FID) as described below.

The peak area of individual FAME were first corrected on the basis of the peak area responses of a known amount of the same FAMEs present in a commercial standard GLC-411 (NU-CHEK PREP, INC., USA). GLC-411 contains equal amounts of 31 fatty acids (% by weight), ranging from C8:0 to C22:6. In case of fatty acids which were not present in the standard, the peak area responses of the most similar FAME was taken. For example, the peak area response of FAMEs of 16:1d9 was used for 16:1d7 and the FAME response of C22:6 was used for C22:5. The corrected areas were used to calculate the mass of each FAME in the sample by comparison to the internal standard mass. Oil is stored mainly in the form of TAG and its weight was calculated based on FAME weight. Total moles of glycerol was determined by calculating moles of each FAME and dividing total moles of FAMEs by three. TAG content was calculated as the sum of glycerol and fatty acyl moieties using a relation: % oil by weight=100×((41×total mol FAME/3)+(total g FAME−(15×total mol FAME)))/g seed, where 41 and 15 are molecular weights of glycerol moiety and methyl group, respectively.

### Analysis of Fatty Acid Content in Larger Seeds

To determine fatty acid composition in single seeds that were larger, such as canola and Camelina seeds, or Sorghum or corn seeds, direct methylation of fatty acids in the seed was performed as for Arabidopsis seeds except with breaking of the seed coats. This method extracted sufficient oil from the seed to allow fatty acid composition analysis. To determine the fatty acid composition of total extracted lipid from seeds, seeds were crushed and lipids extracted with CHCl₃/MeOH. Aliquots of the extracted lipid were methylated and analysed by GC. Pooled seed-total lipid content (seed oil content) of canola was determined by two extractions of lipid using CHCl₃/MeOH from a known weight of desiccated seeds after crushing, followed by methylation of aliquots of the lipids together with the 17:0 fatty acids as internal standard. In the case of larger seeds such as Camelina, the lipid from a known amount of seeds was methylated together with known amount of 17:0 fatty acids as for the Arabidopsis oil analysis and FAME were analysed by GC. For TAG quantitation, TAG was fractionated from the extracted lipid using TLC and directly methylated in silica using 17:0 TAG as an internal standard. The methods are described as follows.

After harvest at plant maturity, seeds were desiccated by storing the seeds for 24 hours at room temperature in a desiccator containing silica gel as desiccant. Moisture content of the seeds was typically 6-8%. Total lipids were extracted from known weights of the desiccated seeds by crushing the seeds using a mixture of chloroform and methanol (2/1 v/v) in an eppendorf tube using a Reicht tissue lyser (22 frequency/seconds for 3 minutes) and a metal ball. One volume of 0.1M KCl was added and the mixture shaken for 10 minutes. The lower non-polar phase was collected after centrifuging the mixture for 5 minutes at 3000 rpm. The remaining upper (aqueous) phase was washed with 2 volumes of chloroform by mixing for 10 minutes. The second non-polar phase was also collected and pooled with the first. The solvent was evaporated from the lipids in the extract under nitrogen flow and the total dried lipid was dissolved in a known volume of chloroform.

To measure the amount of lipid in the extracted material, a known amount of 17:0-TAG was added as internal standard and the lipids from the known amount of seeds incubated in 1 N methanolic-HCl (Supelco) for 2 hours at 80° C. FAME thus made were extracted in hexane and analysed by GC. Individual FAME were quantified on the basis of the amount of 17:0 TAG-FAME. Individual FAME weights, after subtraction of weights of the esterified methyl groups from FAME, were converted into moles by dividing by molecular weights of individual FAME. Total moles of all FAME were divided by three to calculate moles of TAG and therefore glycerol. Then, moles of TAG were converted in to weight of TAG. Finally, the percentage oil content on a seed weight basis was calculated using seed weights, assuming that all of the extracted lipid was TAG or equivalent to TAG for the purpose of calculating oil content. This method was based on Li et al. (2006). Seeds other than Camelina or canola seeds that are of a similar size can also be analysed by this method.

Canola and other seed oil content can be measured by nuclear magnetic resonance techniques (Rossell and Pritchard, 1991) by a pulsed wave NMS 100 Minispec (Bruker Pty Ltd Scientific Instruments, Germany). The NMR method can simultaneously measured moisture content. Seed oil content can also be measured by near infrared reflectance (NIR) spectroscopy such as using a NIRSystems Model 5000 monochromator. Moisture content can also be measured on a sample from a batch of seeds by drying the seeds in the sample for 18 hours at about 100° C., according to Li et al. (2006).

Analysis of Lipids from Leaf Lysate Assays

Lipids from the lysate assays were extracted using chloroform:methanol: 0.1 M KCl (2:1:1) and recovered. The different lipid classes in the samples were separated on Silica gel 60 thin layer chromatography (TLC) plates (MERCK, Dermstadt, Germany) impregnated with 10% boric acid. The solvent system used to fractionate TAG from the lipid extract was chloroform/acetone (90/10 v/v). Individual lipid classes were visualized by exposing the plates to iodine vapour and identified by running parallel authentic standards on the same TLC plate. The plates were exposed to phosphor imaging screens overnight and analysed by a Fujifilm FLA-5000 phosphorimager before liquid scintillation counting for DPM quantification.

Total Lipid Isolation and Fractionation of Lipids from Vegetative Tissues

Fatty acid composition of total lipid in leaf and other vegetative tissue samples was determined by direct methylation of the fatty acids in freeze-dried samples. For total lipid quantitation, fatty acids in a known weight of freeze-dried samples, with 17:0 FFA, were directly methylated. To determine total TAG levels in leaf samples, TAG was fractionated by TLC from extracted total lipids, and methylated in the presence of 17:0 TAG internal standard, because of the presence of substantial amounts of polar lipids in leaves. This was done as follows. Tissues including leaf samples were freeze-dried, weighed (dry weight) and total lipids extracted as described by Bligh and Dyer (1959) or by using chloroform:methanol: 0.1 M KCl (CMK; 2:1:1) as a solvent. Total lipids were extracted from N. benthamiana leaf samples, after freeze dying, by adding 900 μL of a chloroform/methanol (2/1 v/v) mixture per 1 cm diameter leaf sample. 0.8 μg DAGE was added per 0.5 mg dry leaf weight as internal standard when TLC-FID analysis was to be performed. Samples were homogenized using an IKA ultra-turrax tissue lyser after which 500 μL 0.1 M KCl was added. Samples were vortexed, centrifuged for 5 min and the lower phase was collected. The remaining upper phase was extracted a second time by adding 600 μL chloroform, vortexing and centrifuging for 5 min. The lower phase was recovered and pooled into the previous collection. Lipids were dried under a nitrogen flow and resuspended in 2 μL chloroform per mg leaf dry weight. Total lipids of N. tabacum leaves or leaf samples were extracted as above with some modifications. If 4 or 6 leaf discs (each approx 1 cm² surface area) were combined, 1.6 ml of CMK solvent was used, whereas if 3 or less leaf discs were combined, 1.2 ml CMK was used. Freeze dried leaf tissues were homogenized in an eppendorf tube containing a metallic ball using a Reicht tissue lyser (Qiagen) for 3 minutes at 20 frequency/sec.

### Separation of Neutral Lipids Via TLC and Transmethylation

Known volumes of total leaf extracts such as, for example, 30 μL were loaded on a TLC silica gel 60 plate (1×20 cm) (Merck KGaA, Germany). The neutral lipids were fractionated into the different types and separated from polar lipids via TLC in an equilibrated development tank containing a hexane/DEE/acetic acid (70/30/1 v/v/v/) solvent system. The TAG bands were visualised by primuline spraying, marked under UV, scraped from the TLC plate, transferred to 2 mL GC vials and dried with N₂. 750 μL of IN methanolic-HCl (Supelco analytical, USA) was added to each vial together with a known amount of C17:0 TAG as an internal standard, depending on the amount of TAG in each sample. Typically, 30 μg of the internal standard was added for low TAG samples whilst up to 200 μg of internal standard was used in the case of high TAG samples.

Lipid samples for fatty acid composition analysis by GC were transmethylated by incubating the mixtures at 80° C. for 2 hours in the presence of the methanolic-HCl. After cooling samples to room temperature, the reaction was stopped by adding 350 μl H2O. Fatty acyl methyl esters (FAME) were extracted from the mixture by adding 350 μl hexane, vortexing and centrifugation at 1700 rpm for 5 min. The upper hexane phase was collected and transferred into GC vials with 300 μl conical inserts. After evaporation, the samples were resuspended in 30 μl hexane. One μl was injected into the GC.

The amount of individual and total fatty acids (TFA) present in the lipid fractions was quantified by GC by determining the area under each peak and calculated by comparison with the peak area for the known amount of internal standard. TAG content in leaf was calculated as the sum of glycerol and fatty acyl moieties in the TAG fraction using a relation: % TAG by weigh=100×((41×total mol FAME/3)+(total g FAME−(15×total mol FAME)))/g leaf dry weight, where 41 and 15 are molecular weights of glycerol moiety and methyl group, respectively.

### Capillary Gas-Liquid Chromatography (GC)

FAME were analysed by GC using an Agilent Technologies 7890A GC (Palo Alto, California, USA) equipped with an SGE BPX70 (70% cyanopropyl polysilphenylene-siloxane) column (30 m×0.25 mm i.d., 0.25 μm film thickness), an FID, a split/splitless injector and an Agilent Technologies 7693 Series auto sampler and injector. Helium was used as the carrier gas. Samples were injected in split mode (50:1 ratio) at an oven temperature of 150° C. After injection, the oven temperature was held at 150° C. for 1 min, then raised to 210° C. at 3° C. min⁻¹ and finally to 240° C. at 50° C. min⁻¹. Peaks were quantified with Agilent Technologies ChemStation software (Rev B.04.03 (16), Palo Alto, California, USA) based on the response of the known amount of the external standard GLC-411 (Nucheck) and C17:0-Me internal standard.

### Quantification of TAG Via Iatroscan

One μL of lipid extract was loaded on one Chromarod-SII for TLC-FID Iatroscan™ (Mitsubishi Chemical Medience Corporation—Japan). The Chromarod rack was then transferred into an equilibrated developing tank containing 70 mL of a hexane/CHCl₃/2-propanol/formic acid (85/10.716/0.567/0.0567 v/v/v/v) solvent system. After 30 min of incubation, the Chromarod rack was dried for 3 min at 100° C. and immediately scanned on an Iatroscan MK-6s TLC-FID analyser (Mitsubishi Chemical Medience Corporation—Japan). Peak areas of DAGE internal standard and TAG were integrated using SIC-480II integration software (Version: 7.0-E SIC System instruments Co., LTD—Japan).

TAG quantification was carried out in two steps. First, DAGE was scanned in all samples to correct the extraction yields after which concentrated TAG samples were selected and diluted. Next, TAG was quantified in diluted samples with a second scan according to the external calibration using glyceryl trilinoleate as external standard (Sigma-Aldrich).

### Quantification of TAG in Leaf Samples by GC

The peak area of individual FAME were first corrected on the basis of the peak area responses of known amounts of the same FAMEs present in a commercial standard GLC-411 (NU-CHEK PREP, Inc., USA). The corrected areas were used to calculate the mass of each FAME in the sample by comparison to the internal standard. Since oil is stored primarily in the form of TAG, the amount of oil was calculated based on the amount of FAME in each sample. Total moles of glycerol were determined by calculating the number of moles of FAMEs and dividing total moles of FAMEs by three. The amount of TAG was calculated as the sum of glycerol and fatty acyl moieties using the formula: % oil by weight=100×((41×total mol FAME/3)+(total g FAME−(15×total mol FAME)))/g leaf dry weight, where 41 and 15 were the molecular weights of glycerol moiety and methyl group, respectively.

### Total Lipid Extraction and Fatty Acid Profile Analysis

Total lipids were extracted from freeze-dried N. benthamiana leaves. During the extraction of total lipids, TAG 51:0 (tri-C17:0) was added as the internal standard for the quantification of both the TAG and total fatty acid (TFA) contents. Freeze dried leaf tissue was ground to powder in a microcentrifuge tube containing a metallic ball using Reicht tissue lyser (Qiagen) for 3 min. at 20 frequency/s. Chloroform:methanol (2:1, v/v) was added and mixed for a further 3 min. on the tissue lyser before the addition of 1:3 (v/v) of 0.1 M KCl. The sample was then mixed for a further 3 min. before centrifugation (5 min. at 14,000 g), after which the lower lipid phase was collected. The remaining phase was washed once with chloroform, and the lower phase extracted and pooled with the earlier extract. Lipid phase solvent was then evaporated completely using N2 gas flow and the lipids resuspended in 5 μL chloroform per mg of original dry leaf weight.

Fatty acid methyl esters (FAMEs) of total lipids (equivalent to 10 mg dry weight) were produced by incubating extracted lipid in 1 N methanolic-HCl (Supelco, Bellefonte, PA) at 80° C. for 3 hours. FAMEs were analyzed by an Agilent 7890A gas chromatograph coupled with flame ionisation detector (GC-FID, Agilent Technologies, Palo Alto, CA), on a BPX70 column (30 m, 0.25 mm inner diameter, 0.25 μm film thickness, SGE) essentially as described previously (Zhou et al., 2011), except the column temperature program. The column temperature was programmed as an initial temperature at 100° C. holding for 3 min, ramping to 240° C. at a rate of 7° C./min and holding for 1 min. NuChek GLC-426 was used as the external reference standard. Peaks were integrated with Agilent Technologies ChemStation software (Rev B.04.03 (16)).

### TLC Analysis

From the total lipid extracts (equivalent to 10 mg dry weight of plant tissue), TAG and polar lipids were fractionated by TLC (Silica gel 60, MERCK) using hexane:diethylether:acetic acid (70:30:1 v/v/v) and visualized by spraying Primuline (Sigma, 5 mg/100 ml acetone:water (80:20 v/v)) and exposing plate under UV. TLC analysis was primarily used for the identification of fatty acid composition of TAG and phospholipids from lipid extraction samples. This also enabled the determination of the total TAG content for each sample. The TAG and phospholipid fractions were scraped from the TLC plates and methylated according to the FAME preparation protocol described previously.

### LC-MS Analysis

Lipids extracted from 1 mg dry leaf weight were dissolved and diluted to 1 mg/ml in mL butanol:methanol (1:1, v/v) and analyzed by liquid chromatography-mass spectrometry (LC-MS), based on previously described methods (Petrie et al., 2012). Briefly, lipids were chromatographically separated using a Waters BEH C8 (100 mm×2.1 mm, 2.7 μm) fitted to an Agilent 1290 series LC and 6490 triple quadrupole LC-MS with Jet Stream ionisation with a binary gradient flow rate of 0.2 mL/min. The mobile phases were: A. H₂O:acetonitrile (10:90, v/v) with 10 mM ammonium formate and 0.2% acetic acid; B. H₂O:acetonitrile:isopropanol (5:15:80, v/v) with 10 mM ammonium formate and 0.2% acetic acid. For the phosphatidylcholine (PC) and lysophosphatidylcholine (LPC) species hydrogen adducts were quantified by the characteristic 184 m z phosphatidyl head group ion under positive ionisation mode. The ammonium adducts of monogalactosyl diacylglycerol (MGDG), digalactosyl diacylglycerol (DGDG), diacylglycerol (DAG) and TAG lipid species were analyzed by the neutral loss of singular fatty acids C12 to C18. Multiple reaction monitoring (MRM) lists were based on the following major fatty acids: 12:0, 14:0, 16:0, 16:3, 18:0, 18:1, 18:2, 18:3, using a collision energy of 28 V for all lipid classes except for DAG where a collision energy of 14 V was used. Individual MRM TAG was identified based on ammoniated precursor ion and production from neutral loss.

### Example 2. Modifying Traits in Vegetative Parts of Monocotyledonous Plants

Chimeric DNA constructs were designed to increase oil content in monocotyledonous plants, for example the C4 plant S. bicolor (sorghum), by expressing a combination of genes encoding WRI1, Z. mays LEC1 (Accession number AAK95562; SEQ ID NO:32), DGAT and Oleosin in the transgenic plants. Several pairs of constructs for biolistic co-transformation were designed and produced by restriction enzyme-ligation cloning, as follows.

The genetic construct pOIL 136 was a binary vector containing three monocot expression cassettes, namely a selectable marker gene encoding phosphinothricin acetyltransferase (PAT) for plant selection, a second cassette for expressing DGAT and a third for expressing Oleosin. pJP136 was first produced by amplifying an Actin-1 gene promoter from Oryza sativa (McElroy et al., 1990) and inserting it as a blunt-(Val fragment into pORE04 (Coutu et al., 2007) to produce pOIL094. pOIL095 was then produced by inserting a version of the Sesamum indicum Oleosin L gene which had been codon optimised for monocot expression into pOIL094 at the KpnI site. pOIL093 was produced by cloning a monocot (Triticum aestivum) codon optimised version of the Umbelopsis ramanniana DGAT2a gene (Lardizabal et al., 2008) as a SmaI-KpnI fragment into a vector already containing a Zea mays Ubiquitin gene promoter. pOIL 134 was then produced by cloning the NotI DGAT2a expression cassette from pOIL093 into pOIL095 at the NotI sites. pOIL141 was produced by inserting the selectable marker gene coding for PAT as a BamHI-SacI fragment into a vector containing the Z. mays Ubiquitin-1 promoter. Finally, pOIL 136 was produced by cloning the Z. mays Ubiquitin:: PAT expression cassette as a blunt-AscI fragment into the ZraI-AscI of pOIL096. The genetic construct pOIL 136 therefore contained the following expression cassettes: promoter O. sativa Actin::S. indicum Oleosin, promoter Z. mays Ubiquitin:: (J. ramanniana DGAT2a and promoter Z. mays Ubiquitin::PAT.

A similar vector pOIL197, containing NPTII instead of PAT was constructed by subcloning of the Z. mays Ubiquitin:NPTII cassette from pUKN (Liu and Godwin, 2012) as a HindIII-SmaI fragment into the AscI (blunted) and HindIII sites of pJP3343. The resulting vector, pOIL196, was then digested with HindIII (blunted) and AgeI. The resulting 3358 bp fragment was cloned into the ZraI-AgeI sites of pOIL 134, yielding pOIL 197.

A set of constructs containing genes encoding the Z. mays WRI1 (ZmWRI) or the LEC1 (ZmLEC1) transcription factors under the control of different promoters were designed and produced for biolistic co-transformation in combination with pOIL 136 or pOIL 197 to test the effect of promoter strength and cell specificity on the function of WRI1 or LEC1, or both if combined, when expressed in vegetative tissues of a C4 plant such as sorghum. This separate set of constructs did not contain a selectable marker gene, except for pOIL333 which contained NPTII as selectable marker. The different promoters tested were as follows. The Z. mays Ubiquitin gene promoter (pZmUbi) was a strong constitutive monocot promoter while the enhanced CaMV 35S promoter (e35S) having a duplicated enhancer region was reported to result in lower transgene expression levels (reviewed in Girijashankar and Swathisree, 2009). Whilst the Z. mays phosphoenolpyruvate carboxylase (pZmPEPC) gene promoter was active in leaf mesophyl cells (Matsuoka and Minami, 1989), the site of photosynthesis in C4 plant species, the Z. mays Rubisco small subunit (pZmSSU) gene promoter was specific for the bundle sheath cell layer (Nomura et al., 2000; Lebrun et al., 1987), the cells where carbon fixation takes place in C4 plants.

The expression of the Z. mays gene encoding the SEE1 cysteine protease (Accession number AJ494982) was identified as similar to that of the A. thaliana SAG12 senescence-specific promoter during plant development. Therefore a 1970 bp promoter from the SEE1 gene (SEQ ID NO:53) was also selected to drive expression of the genes encoding the Z. mays WRI1 and LEC1 transcription factors. Further, the promoter from the gene encoding Aeluropus littoralis zinc finger protein A1SAP (Ben Saad et al., 2011; Accession number DQ885219; SEQ ID NO:54), the promoter from the gene encoding the Saccharum hybrid DIRIGENT (DIR16) (Damaj et al., 2010; Accession number GU062718; SEQ ID NO:82), the promoter from the gene encoding the Saccharum hybrid O-Methyl transferase (OMT) (Damaj et al., 2010; Accession number GU062719; SEQ ID NO:83), the A1 promoter allel from the gene encoding the Saccharum hybrid R1MYB1 (Mudge et al., 2013; Accession number JX514703.1; SEQ ID NO:84), the promoter from the gene encoding the Saccharum hybrid Loading Stem Gene 5 (LSG5) (Moyle and Birch, 2013; Accession number JX514698.1; SEQ ID NO:85) and the promoter from the sucrose-responsive ArRolC gene from A. rhizogenes (Yokoyama et al., 1994; Accession number DQ160187; SEQ ID NO:55) were also selected for expression of ZmWRI1 expression in stem tissue. Therefore, each of these promoters was individually joined upstream of the ZmWRI1 or ZmLEC1 coding regions, as follows.

An intermediate vector, pOIL100, was first produced by cloning the Z. mays WRI1 coding sequence and a Glycine max lectin gene transcription terminator/polyadenylation region, flanked by AscI-NcoI sites, into the same sites in the binary vector pJP3343. The WRI1 coding sequence was codon optimized using T. aestivum codon preferences. The different versions of the constructs for WRI1 expression were based on pOIL 100 and were produced by cloning the various promoters into pOIL 100. pOIL 101 was produced by cloning a XhoI-Sa/I fragment containing the e35S promoter with duplicated enhancer region into the XhoI site of pOIL100. pOIL102 was produced by cloning a HindIII-AvrII fragment containing the Z. mays Ubiquitin gene promoter (Christensen et al., 1992) into the HindIII-XbaI sites of pOIL100. pOIL 103 was produced by cloning a HindIII-NcoI fragment containing a Z. mays PEPC gene promoter (Matsuoka and Minami, 1989) into the HindIII-NcoI sites of pOIL 100. pOIL104 was produced by cloning a HindIII-AvrII fragment containing a Z. mays SSU gene promoter into the HindIII-AvrII sites of pOIL100.

A synthetic fragment containing the Z. mays SEE1 promoter region flanked by HindIII-XhoI unique sites was synthesized. This fragment was cloned upstream of the Z. mays WRI1 protein coding region using the HindIII-XhoI sites in pOIL 100. The resulting vector was designated pOIL329. A synthetic fragment containing the A. littoralis AlSAP promoter region flanked by XhoI-XbaI unique sites was synthesized. This fragment was cloned upstream of the Z. mays WRI1 coding region using the XbaI-XhoI sites in pOIL 100. The resulting vector was designated pOIL330. A synthetic fragment containing the A. rhizogenes ArRolC promoter region flanked by PspOMI-XhoI unique sites was synthesized. This fragment was cloned upstream of the Z. mays WRI1 coding region using the PspOMI-XhoI sites in pOIL 100. The resulting vector was designated pOIL335. Finally, a binary vector (pOIL333) containing the Z. mays SEE1::ZmLEC1 expression cassette was obtained in three steps. First, a 35S::GUS expression vector was constructed by amplifying the GUS coding region with flanking primers containing AvrII and KpnI sites. The resulting fragment was subsequently cloned into the SpeI-KpnI sites of pJP3343. The resulting vector was designated pTV111. Next, the 35S promoter region of pTV111 was replaced by the Z. mays SEE1 promoter. To this end, the Z. mays SEE1 sequence was amplified using flanking primers containing HindIII and XhoI unique sites. The resulting fragment was cut with the respective restriction enzymes and subcloned into the SalI-HindIII sites of pTV111. The resulting vector was designated pOIL332. Next the ZmLEC1 coding sequence was amplified using flanking primers containing NotI and EcoRV sites. This resulting fragment was subcloned into the respective sites of pOIL332, yielding pOIL333.

A 2673 bp synthetic fragment containing the Saccharum DIR16 promoter region flanked by HindIII-XbaI sites was synthesized. This fragment was cloned upstream of the Z. mays WRI1 protein coding region using the HindIII-XbaI sites in pOIL 100. The resulting vector was designated pOIL337. A 2947 bp synthetic fragment containing the Saccharum OMT promoter region flanked by XhoI-XbaI sites was synthesized. This fragment was cloned upstream of the Z. mays WRI1 protein coding region using the XhoI-XbaI sites in pOIL100. The resulting vector was designated pOIL339. A 1181 bp synthetic fragment containing the Saccharum R1MYB1 promoter region flanked by HindIII-XhoI sites was synthesized. This fragment was cloned upstream of the Z. mays WRI1 protein coding region using the HindIII-XhoI sites in pOIL 100. The resulting vector was designated pOIL341. A 4482 bp synthetic fragment containing the Saccharum LSG5 promoter region flanked by XbaIII-SmaI sites was synthesized. This fragment was cloned as an XbaIII-SmaI fragment upstream of the Z. mays WRI1 protein coding region using the StuI-NheI sites in pOIL100. The resulting vector was designated pOIL343.

Two putative S. bicolor SDP1 genes were identified by a BLASTn search using an A. thaliana SDP 1 nucleotide sequence (Accession number NM_120486; SEQ ID NO:37) as query. The Accession numbers of the two S. bicolor SDP1 homologs are XM_002458486 (SEQ ID NO:38) and XM 002463620 (SEQ ID NO:73). A 7991 bp synthetic fragment was synthesized and contained the following genetic components in order: a matrix association region (MAR), a Z. mays promoter, a TMV 5′ UTR sequence, a 2198 bp hairpin RNA encoding region (SEQ ID NO:75) directed against both S. bicolor SDP1 genes, an OCS gene polyadenylation/transcription terminator, an O. sativa Actin-1 gene promoter, TMV 5′ UTR sequence, and a NOS gene polyadenylation/transcription terminator. The hairpin RNA encoding region contained a Pdk intron (Wesley et al., 2001) and a Cat intron, the second in reverse orientation. The entire fragment was synthesized and inserted into an E. coli expression vector. The resulting vector was designated pOIL385.

Whole plasmid DNA was prepared from pOIL101, pOIL 102, pOIL 103, pOIL104, pOIL197, pOIL 136, pOIL329 and pOIL385 for biolistic transformation. pOIL197 DNA was then mixed with DNA from either pOIL101, pOIL 102, pOIL 103, pOIL 104, pOIL329 or pOIL385 and introduced by biolistics into S. bicolor (TX430) differentiating embryonic calli (DEC) cells to produced transformed plants as described in Example 1. Alternatively, constructs for expression of the same combinations of genes are introduced separately or co-transformed by Agrobacterium-mediated methods (Gurel et al., 2009; Wu et al., 2014) into DEC tissues.

Between 9 and 47 transgenic plants were regenerated and selected by antibiotic resistance for the pairs of constructs including pOIL197 with each of pOIL101 (p35SSWRI1); pOIL102 (pZmUbi::WRI1), pOIL103 (pZmPEPC::WRI1), pOIL104 (pSSU::WRI1) and pOIL329 (pSEE1::WRI1). Transformations were also carried out with pOIL 197 or pOIL 102 alone, and for the transformation vector without an insert (empty vector control). The presence of the introduced transgenes in plants that were resistant to the selective agent was demonstrated by PCR. The copy number of each transgene was also determined by digital droplet PCR (ddPCR).

Total leaf lipids were quantified in a first subset of transgenic S. bicolor plants prior to their transfer from MS medium to soil. This preliminary screening suggested slightly elevated total lipid levels in leaf tissue of some events at this very early stage. The line with the highest total lipid content, pOIL 136 (2), was further analyzed by thin layer chromatography (TLC) to determine the effect of transgene expression on TAG accumulation. Leaf tissue of this particular line was sampled at vegetative stage following transfer to soil in the glasshouse. When compared to the wildtype and empty vector negative controls, pOIL136 (2) exhibited increased TAG levels in leaf tissue after TLC separation and iodine staining. Subsequent quantification revealed 10-fold increased TAG in the transgenic line compared to the negative controls. The TAG profile was dominated by the polyunsaturated fatty acids linoleic and α-linolenic acid. The presence or absence of all three transgenes was determined by digital PCR analysis. Of note, up to 30% mortality rate was observed for plantlets at rooting stage during tissue culture following transformation with the pOIL 103 and pOIL 197 combination due to unknown reasons.

Confirmed transgenic plants were transferred to soil in pots in the glasshouse and leaves were sampled from primary transformants at vegetative stage of growth (i.e. prior to the appearance of the boot leaf), at the boot leaf stage (defined as when the boot leaf has fully emerged, the boot leaf is the last leaf formed on the plant and from which the panicle (head) emerges) and at the mature seed-setting stage. Total fatty acid (TFA) and triacylglycerol (TAG) contents (% leaf dry weight) were quantified by TLC-GC as described in Example 1.

TFA levels in wild-type and empty vector negative controls decreased during plant development and were in the range 0.05-2.9% (weight/dry weight). The highest TFA levels were detected prior to the appearance of the boot leaf (termed the vegetative stage of growth) and were below 3%. TAG levels in the same plants were consistently low in the range 0-0.2% during the entire plant life cycle. Both the TFA content and the TAG content had fatty acid compositions of predominantly C16:0, C18:2^{Δ9,12}(LA) and C18:3^{Δ9,12,15}(ALA). In particular, ALA was present at >70% of the TFA content, reflecting use of this fatty acid in wild-type plastid membranes. ALA also was the predominant fatty acid in the small amount of TAG present in the wild-type leaves.

27 confirmed transgenic plants which had been transformed with pOIL197 or pOIL136, comprising both pZmUbi:DGAT and pZmUbi:Oleosin genes in addition to the selectable marker genes, were analysed at the vegetative, boot leaf and mature seed setting stages. Some data are presented in Table 5. Generally, the pOIL 197 and pOIL 136 primary transformants displayed increased TFA and TAG accumulation compared to the negative control lines, but only to about triple for the TFA level compared to the controls. The highest TFA levels were detected at the vegetative stage of growth. Similar to the wild-type and negative control lines, TFA levels decreased as the plants grew and developed. Maximum TFA levels at vegetative, boot leaf and mature seed setting stages equalled 4.3%, 3.3% and 2.2%, respectively. The highest TAG levels detected varied between 0.8 and 1.4% depending on the age of the plant at the time of sampling (Table 3), so were increased up to 7-fold relative to the very low levels in the wild-type leaves. The TFA composition remained largely unchanged at the different stages and was dominated by ALA. The TAG composition displayed a higher degree of variation between the different transgenic lines. Compared to the fatty acid composition of the TFA content, the level of LA (18:2^{Δ9,12}) was consistently increased in TAG throughout all plant stages investigated.

Nine primary regenerated plants made by transformation with the single vector pOIL 102 (pZmUbi:WRI1) were generated by co-bombardment of pOIL 102 and pUKN, containing the NPTII selectable marker gene. Table 4 shows some of the data for TFA and TAG contents and fatty acid compositions were measured at the three growth stages. When compared to the plants transformed with the constructs encoding DGAT2 and Oleosin (pOIL 197 or pOIL136), TFA and TAG levels in the pOIL 102 transgenic events were generally lower. Indeed, levels of TFA and TAG were similar to the levels in the wild-type and negative control plants at vegetative stage. Maximum TFA levels at vegetative, boot leaf and mature seed setting stages were 2.6%, 2.5% and 2.0%, respectively (Table 4). Maximum TAG levels observed were 0.2%, 0.4% and 0.9% at vegetative, boot leaf and mature seed setting stages, respectively.

Thirty-seven primary regenerated plants were obtained after co-bombardment with both pOIL 197 (pZmUbi:DGAT and pZmUbi:Oleosin) and pOIL102 (pZmUbi:WRI1). Four of the regenerated events were found to be non-transgenic. In addition, 2 plants did not contain pOIL 102 while 2 other plants did not contain the DGAT2 transgene. All of the transgenic plants were analysed for TFA and TAG contents and fatty acid composition at the three growth stages, as above. Representative data are presented in Table 5. Some of the plants exhibited greatly increased TFA and TAG levels compared to the plants transformed with single vectors pOIL 197, pOIL 136 or pOIL102. The maximum TFA levels at vegetative, boot leaf and mature seed setting stages in the pOIL 102+pOIL197 transformed plants equalled 7.2%, 6.4% and 8.7% (w/dry weight), respectively. Importantly, the maximum observed TAG levels increased during plant development from 2.7% (vegetative stage) to 3.5% (boot leaf stage) and 6.1% (mature seed setting stage). Compared with the data obtained for the separate transformations with the DGAT and WRI1 transgenes, this exemplified the synergism for co-expressing DGAT and WRI1 transgenes to increase non-polar lipid accumulation in vegetative plant tissues. High levels of TAG and TFA were in most cases associated with a substantial reduction in the C18:3^{Δ9,12,15} content, which was reduced by about 50% in the lines with the highest levels of TAG.

Forty-seven primary transformants were obtained following transformation with both pOIL 197 (pZmUbi:DGAT and pZmUbi:Oleosin) and pOIL 103 (pZmPEPC:WRI1). Copy number analysis by ddPCR revealed one non-transgenic plant and 3 plants that did not contain DGAT2 and/or OLEOSIN transgenes. All events were subsequently analysed for TFA and TAG contents and fatty acid composition during the three stages of plant development. Some plants with this gene combination exhibited the highest TFA and TAG levels detected in this experimental series. TFA levels were observed at vegetative, boot leaf and mature seed setting stages in the pOIL103+pOIL 197 population at 8.3%, 8.3% and 9.7%, respectively. Maximum TAG levels observed at vegetative, boot leaf and mature seed setting stages were at 2.3%, 6.6% and 7.6%, respectively. Of note, the highest TAG (6.6%) and TFA (8.3%) levels amongst all transgenic lines were detected in event TX-03-31 at mature seed setting stage. While C18:3^{Δ9,12,15} typically dominated the TFA fraction other than TAG, the TAG in this population of transgenic plants displayed a high degree of variability in fatty acid composition. Of note, some plants exhibited increases in levels of palmitic acid (C16:0) and/or linoleic acid (LA, C18:2^{Δ9,12}) at the expense of ALA. Indeed, the ALA level in both TFA and TAG contents was reduced below 40% in some plants as a percentage of the total fatty acid content, while less than 30% in other selected events. The ALA level in TAG was even less than 20% in some selected plants, as a percentage of the total fatty acid content.

Due to the use of biolistic transformation in this experiment, many of the transgenic sorghum plants contained high transgene copy numbers as determined by digital PCR. In addition, varying degrees of male and female sterility were observed amongst the transgenic lines, likely a result of the multiple transgene insertions. The inventors therefore did not pursue homozygosity of the transgenes in subsequent generations but rather performed a detailed analysis on vegetative progeny plants obtained from selected primary transformants. To this end, tillers were propagated allowing for triplicate analyses of TAG and TFA levels. Furthermore, the analyses focused on the boot leaf stage of growth as this was a distinct and easily identified time point during development that allowed for good comparison between the different transgenic lines, grown under the same environmental conditions. Plants containing the higher levels of TFA and TAG were propagated by separating tillers and transplanting them into soil in new pots. The tillers produced new roots and continued to grow.

Quantitation of the total lipid content in triplicate leaves from established tillers confirmed elevated TAG and TFA contents in several independent lines co-transformed with either pOIL 102+pOIL197 or pOIL 103+pOIL197. The highest levels were observed in progeny plants of line 03-31, confirming the earlier results. Leaves of this line contained on average 6.9% TFA and 4.6% TAG (% DW) at boot leaf stage. This corresponded to an 89.4-fold increase in TAG content compared to wild-type control leaves at the same developmental stage.

Twenty primary regenerated plants were obtained following transformation with both pOIL 197 (pZmUbi:DGAT and pZmUbi:Oleosin) and pOIL 104 (pSSU:WRI1). Five plants were found to be non-transgenic and four other plants had only the gene(s) from one of the genetic constructs. All plants were analysed for TFA and TAG contents and fatty acid composition. Leaves of primary transformants containing both pOIL 197 and pOIL 104 T-DNA regions, sampled at vegetative, mature and seed setting stages of growth contained up to 4.1%, 5.9% and 5.89% TFA, respectively. Surprisingly, the highest TFA levels detected in this population were accompanied by a relatively low TAG content. TAG levels in pOIL104+pOIL197 transgenic plants at vegetative, boot leaf and seed setting stages reached only to 0.7%, 2.8% and 3.4%. Increased TAG levels were typically associated with a reduction in C18:3^{Δ9,12,15} and an increase in both palmitic acid and LA.

Forty-three primary regenerated plants were obtained following transformation with both pOIL 197 (pZmUbi:DGAT and pZmUbi:Oleosin) and pOIL101 (p35S:WRI1). One plant was non-transgenic, another lacked the WRI1 transgene and another lacked the DGAT1 transgene. All plants were analysed for TFA and TAG contents and fatty acid composition at boot leaf stage. Leaves of primary transformants containing both pOIL197 and pOIL104 T-DNA regions contained up to 4% TFA while TAG levels were low with a maximum of 1.4%. Increased TAG levels were associated with a reduction in C18:3^{Δ9,12,15} as a percentage of the total fatty acid content.

Twenty primary transformants were obtained following transformation with both pOIL197 (pZmUbi:DGAT and pZmUbi:Oleosin) and pOIL329 (pSEE1:WRI1). All plants were confirmed to be transgenic by ddPCR. TFA and TAG levels in leaves of 10 plants at vegetative growth stage were increased up to 3.6% and 0.3%, respectively. Maximum TFA and TAG levels at boot leaf stage equalled 3.8% and 1.5%, respectively. The low TFA and TAG levels were likely the result of the senescence-specific expression patterns of the SEE1 promoter used to drive WRI1 transgene expression. Increased TAG levels were typically associated with a reduction in C18:3^{Δ9,12,15} as a percentage of the total fatty acid content.

Thirty-six primary regenerated plants were obtained following transformation with both pOIL 197 (pZmUbi:DGAT and pZmUbi:Oleosin) and pOIL385 (SDP1hpRNAi). Two plants lacked pOIL 197 and another two lacked pOIL385. The highest TFA level detected in transgenic leaves at the vegetative growth stage was 4.2%. TAG levels in this particular event at the same growth stage was only 1.0%. TFA and TAG levels in leaves sampled at boot leaf stage were increased up to 3.9% and 1.6%, respectively. The lower TFA and TAG levels could be due to the absence of a WRI1 transgene in this transgenic population. No changes in TAG or TFA fatty acid composition were detected relative to the wild-type plants.

Transgene expression levels were determined in propagated tillers of selected lines by RT-PCR. In the majority of transgenic lines, the DGAT2a transgene was typically expressed at a higher levels than the WRI1 transgene. Oleosin L gene expression was either low or not detected. Total lipid and TAG contents at the boot leaf stage were used to calculate correlation coefficients with gene expression. Both WRI1 and DGAT2a gene expression showed a significant positive correlation with TAG levels amongst pOIL 102+pOIL197 and pOIL103+pOIL197 transgenic populations. Significant, albeit slightly weaker, correlation was observed for TFA content and WRI1 or DGAT2a expression. Oleosin L expression was not correlated with either TAG or TFA accumulation in transgenic leaf tissues. It was observed that plant TX-03-31 which had a relatively high TTQ had the highest level of expression of DGAT amongst the tested plants. It was concluded that high levels of DGAT expression were beneficial for increasing the TAG level and also the TTQ.

The most surprising and unexpected observation made in these experiments was the relatively high level of TFA accompanied by the low levels of TAG in most of the transformed sorghum plants, except in a few exceptional plants such as plant TX-03-31. That is, although fatty acid synthesis and accumulation were significantly increased, much of the fatty acid was appearing as TFA but not as TAG. This observation was the opposite of what had been seen with the WRI1+DGAT transgenic plants for Nicotiana including tobacco. To quantitate this in the sorghum plants, the quotient of the TAG to TFA level was calculated for all of the above mentioned transgenic sorghum populations (Tables 3-6). The TAG/TFA Quotient (TTQ) parameter was calculated as the level of TAG (%) divided by the level of TFA (%), each as a percentage of the dry weight of the plant material (leaf in this case). It was observed that for many of the sorghum lines, the TTQ was in the range of 0.01 to 0.6, i.e. less than 60% of the TFA was present as TAG. Addition of one or more further genetic modifications to the combination of WRI1 and DGAT genes such as, for example, which provide for a reduction in the expression of endogenous SDP1, TGD or TST genes, or an increase in the levels of one or more of PDAT, PDCT or CPT polypeptides increases the TTQ to above 0.6 for a larger proportion of the plant lines. In particular, reduction in TAG lipase in combination with at least WRI1 and DGAT increases the TTQ to up to 0.95.

Due to the large difference in absolute TFA and TAG levels in many transgenic lines, the inventors selected two pOIL 102+pOIL 197 events (02-10, 02-19) and two pOIL 103+pOIL 197 events (03-31 and 03-48) for quantitation of the major neutral and polar lipid classes, to determine the type of lipid other than TAG in which the high level of fatty acids was present. The types of lipid were separated by TLC and quantitated. The propagated tillers were smaller compared to tillers obtained from wild-type controls plants grown under the same conditions with the exception of line 03-48. Quantitation by GC-FID of TAG and TFA levels in triplicate leaves confirmed increases in both lipid fractions. Maximum average TAG levels in triplicate leaves (% DW) of lines 02-19 and 03-31 sampled at boot leaf stage were 2.8% and 5.2%, respectively. For all of the transgenic lines, linoleic acid was increased at the expense of α-linolenic acid. However, differences were observed in the levels of palmitic acid and oleic acid. Lines 02-10 and 02-19 contained increased proportions of oleic acid, whereas palmitic acid was elevated in the TFA and TAG fractions of 03-31 and 03-48 leaves. Lipid quantitation in leaf and stem tissues at seed setting stage revealed considerable leaf-to-leaf variation. Lower TFA and TAG contents were observed in older leaves of wild-type and transgenic propagated tillers. The TFA and TAG levels in the flag leaf of line 03-31 at seed setting equalled 9.9% and 8.4% on a DW basis, respectively. Transgenic stem tissues contained up to about 3% total lipids on a dry weight basis compared to 0.3% in wild-type stems.

Total lipid extracts from the wild-type and transgenic leaves sampled at boot leaf stage were subjected to LC-MS to analyse different neutral and polar lipid classes in more detail. Plants of all four transgenic lines exhibited elevated TAG, amounting to a 100-fold increase in line 03-31 compared to the wild-type control leaves. Small increases in levels of PC were detected in plants of the 03-31 and 03-48 transgenic lines while levels of the plastidial galactolipids MGDG and DGDG were variable, increased in some, decreased in other plants. Both LPC and DAG constituted minor lipid classes. TAG molecular species in plants of lines 03-31 and 03-48 were enriched in palmitic acid and linoleic acid. Major TAG species included TAG (50:2) and TAG (50:3) which contained two palmityl groups and TAG (52:4) and TAG (52:5) which contained palmitoyl and linoloyl groups. In contrast, plants of lines 02-10 and 02-19 exhibited distinctly different TAG profiles. Leaf tissues of both lines preferentially accumulated TAG comprising one or more linolyl chains such as TAG (52:3-5) and TAG (54:4-8). The distinct differences in TAG profiles between the two transgenic populations were consistent with earlier GC-FID results.

Changes in TAG compositions were also reflected in the precursor DAG. Dominant DAG (34:2) and DAG (34:3) molecular species in plants 03-31 and 03-48 were enriched in palmitic acid while both 02-10 and 02-19 plants had DAG molecules containing two C18 acyl chains (DAG 36:2-6). Abundant eukaryotic galactolipid species such as MGDG (36:6) and DGDG (36:6) were either reduced or not significantly affected. Two prokaryotic galactolipid species, MGDG (34:3) and DGDG (34:2) were increased slightly in plants 03-31 and 03-48. The dominant prokaryotic DGDG species (34:3) was either unchanged or reduced in transgenic leaves. PC molecular species containing palmitic or linoleic acid including PC (34:1-2) and PC (36:4) were elevated, particularly in lines 03-31 and 03-48. Di-palmitoyl PC (32:0) was increased in line 03-31, reflecting the higher levels of palmitic acid as detected by GC-FID.

Taken together, these results indicated an increased flux of acyl chains into TAG from PC in the transgenic lines whilst galactolipid biosynthesis mainly occurred via the eukaryotic pathway. These data also led the inventors to understand that reduction of TGD activity or increases in PDCT and/or CPT in the plants in addition to the present transgenes would likely enhance the TFA and TAG levels.

### TAG Accumulation Affects Starch and Amino Acid Content

Transitory starch levels in transgenic leaves of lines 03-31 and 03-48 were reduced 7.4- and 15.3-fold on average, respectively. In contrast, starch levels in leaves of 02-10 and 02-19 plants were not significantly affected. Sucrose constituted the dominant leaf soluble sugar in all plants. Sucrose levels were 2-fold lower in line 03-48 while similar to the wild-type control in line 02-19. Raffinose was reduced by 19.6-fold in line 03-48 while monosaccharides such as glucose, fructose and galactose displayed smaller reductions.

A metabolite quantitation by GC-MS identified 36 compounds that were significantly different in leaves of wild-type and transgenic plants. Twenty metabolites were detected at higher levels in TAG-accumulating leaves, including multiple amino acids, urea and the citric acid cycle (TCA) intermediate, α-ketoglutarate. Several dicarboxylic acids, sugar alcohols, fructose, xylose and shikimate were amongst the metabolites that were less abundant in transgenic leaves. Principle component analysis revealed clear separations of both transgenic events and the wild-type control.

### Sorghum Leaves Accumulate TAG as Cytosolic Lipid Droplets

To examine transgenic leaves microscopically to see whether the increased TAG was accumulated in oil droplets, flag leaves of re-established side tillers from transgenic S. bicolor plants were harvested at the beginning of flowering and kept on ice until sections were prepared for imaging. Fresh, thin hand sections were stained for 10 min with a solution of 50 mM PIPES pH7 supplemented with 2 μg/ml of BODIPY 505-515 (4,4-Difluoro-1,3,5,7-Tetramethyl-4-Bora-3a,4a-Diaza-s-Indacene, ThermoFisher Scientific). They were then rinsed in a solution of PIPES pH7 and imaged right away. Control sections were placed directly in PIPES pH7 for 10 min before being mounted on slides and imaged.

All samples were imaged with a confocal laser scanning microscope (Leica TCS SP8) equipped with a white light laser and a 40× water immersion objective ([NA]=1.1), and controlled by the LAS X software (Leica Microsystems). Imaging was done in a sequential manner: BODIPY was excited at 505 nm and its emission was collected at 520-540 nm, while in a separate track, chloroplasts were excited at 633 nm and their auto-fluorescence was collected at 650-690 nm. Maximum projections were generated with the LAS X software. Confocal imaging settings were optimized to distinguish cell types in which oil accumulated by minimizing chloroplast auto-fluorescence in the bundle sheath cells as opposed to the surrounding mesophyll cells.

Leaf cross sections of line 03-10 revealed an abundance of small lipid droplets that preferentially accumulated in the cytosol of mesophyll cells. The unequal distribution likely reflected the tissue specificity of the PEPC promoter used to generate this particular transgenic line. Some lipid accumulation was also visible in the bundle sheath cells of transgenic lines and the wild-type control. Line 02-10 contained an intermediate number of lipid droplets, confirming previous LC-MS and GC-FID TAG quantitation results. Transmission electron micrographs showed densely packed small lipid droplets in the cytosol of mesophyll cells in line 03-31. Mesophyll cells of the wild-type control plants were largely devoid of cytosolic oil droplets.

The chimeric DNA constructs for Agrobacterium-mediated transformation are used to transform Zea mays (corn) as described by Gould et al. (1991). Briefly, shoot apex explants are co-cultivated with transgenic Agrobacterium for two days before being transferred onto a MS salt media containing kanamycin and carbenicillin. After several rounds of sub-culture, transformed shoots and roots spontaneously form and are transplanted to soil. The constructs are similarly used to transform Hordeum vulgare (barley) and Avena sativa (oats) using transformation methods known for these species. Briefly, for barley, the Agrobacterium cultures are used to transform cells in immature embryos of barley (cv. Golden Promise) according to published methods (Tingay et al., 1997; Bartlett et al., 2008) with some modifications in that embryos between 1.5 and 2.5 mm in length are isolated from immature caryopses and the embryonic axes removed. Resulting explants are co-cultivated for 2-3 days with the transgenic Agrobacterium and then cultured in the dark for 4-6 weeks on media containing timentin and hygromycin to generate embryogenic callus then moved to transition media in low light conditions for two weeks. Calli are then transferred to regeneration media to allow for the regeneration of shoots and roots before transfer of the regenerated plantlets to soil. Transformed plants are obtained and grown to maturity in the glasshouse.

TABLE 3
TFA and TAG levels, fatty acid composition and TTQ in sorghum leaves
transformed with pOIL197 or pOIL136 (pZmUbi:DGAT; pZmUbi:Oleosin)
during the boot leaf stage of growth. The lines are listed in order of
increasing TFA levels.

| Line | TAG or TFA | C16:0 | C18:0 | C18:1 | C18:2 | C18:3n3 | Other | TFA | TAG | TTQ |
|---|---|---|---|---|---|---|---|---|---|---|
| TX-197-14 | TFA | 12.7 | 5.2 | 2.0 | 14.4 | 57.7 | 8.1 | 1.2 | | |
| TX-197-14 | TAG | 8.8 | 7.1 | 3.1 | 22.7 | 54.7 | 3.6 | | 0.3 | 0.266 |
| TX-197-15 | TFA | 14.5 | 5.0 | 2.3 | 14.7 | 55.8 | 7.7 | 1.2 | | |
| TX-197-15 | TAG | 12.7 | 7.1 | 3.2 | 21.0 | 51.7 | 4.3 | | 0.3 | 0.262 |
| TX-197-19 | TFA | 13.1 | 3.2 | 2.0 | 14.3 | 60.9 | 6.4 | 1.2 | | |
| TX-197-19 | TAG | 10.6 | 4.3 | 3.4 | 24.4 | 54.0 | 3.2 | | 0.2 | 0.203 |
| TX-136-03 | TFA | 14.1 | 1.8 | 1.7 | 12.6 | 65.0 | 4.8 | 1.2 | | |
| TX-136-03 | TAG | 14.5 | 4.3 | 4.5 | 32.9 | 42.2 | 1.6 | | 0.1 | 0.045 |
| TX-197-08 | TFA | 14.4 | 3.5 | 1.3 | 14.2 | 62.2 | 4.4 | 1.2 | | |
| TX-197-08 | TAG | 13.7 | 5.2 | 2.7 | 22.4 | 50.5 | 5.5 | | 0.3 | 0.211 |
| TX-197-11 | TFA | 14.1 | 3.8 | 2.0 | 15.0 | 57.0 | 8.2 | 1.3 | | |
| TX-197-11 | TAG | 10.3 | 4.8 | 3.0 | 22.8 | 55.9 | 3.1 | | 0.3 | 0.267 |
| TX-136-24 | TFA | 15.5 | 2.2 | 2.2 | 16.9 | 58.1 | 5.2 | 1.3 | | |
| TX-136-24 | TAG | 14.7 | 3.3 | 4.0 | 32.4 | 42.9 | 2.7 | | 0.2 | 0.164 |
| TX-136-02 | TFA | 12.3 | 1.5 | 1.4 | 14.7 | 65.7 | 4.4 | 1.5 | | |
| TX-136-02 | TAG | 13.9 | 2.7 | 3.0 | 28.7 | 46.6 | 5.1 | | 0.7 | 0.444 |
| TX-197-30 | TFA | 13.1 | 2.3 | 1.3 | 9.3 | 65.1 | 8.8 | 2.0 | | |
| TX-197-30 | TAG | 10.0 | 3.0 | 2.2 | 15.0 | 65.3 | 4.5 | | 0.4 | 0.223 |
| TX-197-46 | TFA | 13.2 | 2.5 | 0.8 | 7.9 | 71.2 | 4.5 | 2.0 | | |
| TX-197-46 | TAG | 17.3 | 18.6 | 3.2 | 14.7 | 42.5 | 3.7 | | 0.1 | 0.033 |
| TX-197-45 | TFA | 13.6 | 2.7 | 0.6 | 6.7 | 71.7 | 4.5 | 2.1 | | |
| TX-197-45 | TAG | 22.7 | 17.7 | 4.4 | 12.9 | 38.6 | 3.6 | | 0.1 | 0.030 |
| TX-197-39 | TFA | 12.6 | 3.6 | 1.1 | 9.0 | 66.2 | 7.4 | 2.1 | | |
| TX-197-39 | TAG | 9.5 | 4.0 | 1.6 | 12.8 | 66.7 | 5.5 | | 0.6 | 0.291 |
| TX-197-22 | TFA | 13.6 | 2.0 | 0.8 | 7.3 | 71.3 | 4.9 | 2.1 | | |
| TX-197-22 | TAG | 13.8 | 3.3 | 1.8 | 14.2 | 64.6 | 2.3 | | 0.1 | 0.056 |
| TX-197-34 | TFA | 12.0 | 3.2 | 1.2 | 9.6 | 67.9 | 5.9 | 2.2 | | |
| TX-197-34 | TAG | 9.1 | 4.6 | 2.3 | 18.4 | 63.2 | 2.3 | | 0.4 | 0.190 |
| TX-197-50 | TFA | 13.0 | 2.5 | 1.1 | 9.1 | 66.8 | 7.5 | 2.5 | | |
| TX-197-50 | TAG | 11.4 | 4.6 | 2.1 | 15.3 | 59.8 | 6.9 | | 0.5 | 0.183 |
| TX-197-43 | TFA | 12.4 | 2.3 | 0.7 | 8.0 | 71.9 | 4.7 | 2.5 | | |
| TX-197-43 | TAG | 11.0 | 4.4 | 1.8 | 15.7 | 62.3 | 4.8 | | 0.2 | 0.065 |
| TX-197-32 | TFA | 12.5 | 2.1 | 1.1 | 9.0 | 70.0 | 5.3 | 2.5 | | |
| TX-197-32 | TAG | 12.8 | 3.7 | 2.1 | 16.1 | 60.3 | 5.0 | | 0.6 | 0.220 |
| TX-197-33 | TFA | 12.1 | 2.7 | 0.7 | 7.9 | 71.0 | 5.6 | 2.5 | | |
| TX-197-33 | TAG | 11.1 | 4.8 | 1.4 | 15.4 | 62.4 | 4.9 | | 0.3 | 0.130 |
| TX-197-41 | TFA | 12.8 | 1.9 | 0.7 | 8.1 | 72.8 | 3.7 | 2.6 | | |
| TX-197-41 | TAG | 15.1 | 5.9 | 2.4 | 16.7 | 53.7 | 6.3 | | 0.2 | 0.065 |
| TX-197-36 | TFA | 12.2 | 2.0 | 0.8 | 7.7 | 71.6 | 5.6 | 2.6 | | |
| TX-197-36 | TAG | 11.4 | 3.4 | 1.6 | 13.9 | 65.6 | 4.1 | | 0.4 | 0.158 |
| TX-197-42 | TFA | 12.4 | 2.1 | 0.8 | 8.2 | 70.3 | 6.3 | 2.7 | | |
| TX-197-42 | TAG | 12.4 | 5.4 | 2.3 | 17.8 | 57.1 | 5.0 | | 0.2 | 0.060 |
| TX-197-51 | TFA | 13.6 | 2.1 | 1.0 | 9.9 | 66.8 | 6.6 | 2.7 | | |
| TX-197-51 | TAG | 13.1 | 4.6 | 3.0 | 18.8 | 53.4 | 7.0 | | 0.5 | 0.175 |
| TX-197-49 | TFA | 15.2 | 2.9 | 1.0 | 9.3 | 65.3 | 6.3 | 2.7 | | |
| TX-197-49 | TAG | 17.3 | 5.0 | 2.0 | 16.7 | 52.7 | 6.3 | | 0.5 | 0.192 |
| TX-197-48 | TFA | 13.0 | 2.3 | 1.0 | 8.8 | 68.5 | 6.4 | 2.8 | | |
| TX-197-48 | TAG | 13.0 | 4.7 | 2.2 | 16.1 | 58.0 | 6.0 | | 0.4 | 0.144 |
| TX-197-38 | TFA | 12.2 | 2.0 | 1.0 | 7.7 | 72.1 | 5.0 | 2.9 | | |
| TX-197-38 | TAG | 11.2 | 3.4 | 2.2 | 14.9 | 63.8 | 4.5 | | 0.5 | 0.160 |
| TX-197-35 | TFA | 12.8 | 1.8 | 0.9 | 8.5 | 69.4 | 6.6 | 2.9 | | |
| TX-197-35 | TAG | 12.7 | 2.9 | 1.7 | 14.5 | 63.3 | 4.9 | | 0.7 | 0.227 |
| TX-197-40 | TFA | 12.7 | 1.9 | 0.7 | 7.7 | 73.9 | 3.1 | 2.9 | | |
| TX-197-40 | TAG | 16.3 | 4.7 | 3.3 | 20.8 | 52.4 | 2.6 | | 0.1 | 0.031 |
| TX-197-47 | TFA | 13.9 | 2.4 | 0.6 | 6.9 | 72.2 | 3.9 | 2.9 | | |
| TX-197-47 | TAG | 24.6 | 19.8 | 5.2 | 10.7 | 34.8 | 4.9 | | 0.0 | 0.017 |
| TX-136-01 | TFA | 11.6 | 1.4 | 1.3 | 14.1 | 67.2 | 4.3 | 3.3 | | |
| TX-136-01 | TAG | 14.6 | 2.9 | 3.0 | 29.5 | 44.1 | 5.9 | | 0.7 | 0.199 |
| TX-197-44 | TFA | 13.5 | 2.1 | 1.4 | 14.7 | 63.1 | 5.1 | 3.4 | | |
| TX-197-44 | TAG | 14.4 | 4.3 | 3.1 | 25.0 | 45.0 | 8.2 | | 0.8 | 0.245 |
| TX-136-25 | TFA | 13.6 | 2.2 | 0.7 | 10.8 | 67.4 | 5.2 | 3.4 | | |
| TX-136-25 | TAG | 16.6 | 4.2 | 1.4 | 20.1 | 51.5 | 6.1 | | 1.0 | 0.286 |
| TX-197-28 | TFA | 11.5 | 1.3 | 0.4 | 7.8 | 75.3 | 3.6 | 3.4 | | |
| TX-197-28 | TAG | 17.4 | 4.5 | 1.6 | 19.5 | 50.2 | 6.9 | | 0.1 | 0.035 |
| TX-197-37 | TFA | 12.6 | 3.4 | 6.3 | 17.4 | 54.1 | 6.2 | 4.5 | | |
| TX-197-37 | TAG | 13.4 | 5.0 | 10.1 | 27.4 | 40.2 | 3.9 | | 1.9 | 0.426 |

TABLE 4
TFA and TAG levels, fatty acid composition and TTQ in sorghum leaves
transformed with pOIL102 (pZmUbi:WRI1) during the boot leaf stage of growth.

| Line | TAG or TFA | C16:0 | C18:0 | C18:1 | C18:2 | C18:3n3 | Other | TFA | TAG | TTQ |
|---|---|---|---|---|---|---|---|---|---|---|
| TX-102-8 | TFA | 16.9 | 4.2 | 2.3 | 12.3 | 57.7 | 6.5 | 0.9 | | |
| TX-102-8 | TAG | 14.5 | 6.2 | 13.5 | 25.7 | 36.8 | 3.4 | | 0.2 | 0.243 |
| TX-102-4 | TFA | 17.1 | 4.2 | 2.0 | 12.5 | 57.5 | 6.7 | 0.9 | | |
| TX-102-4 | TAG | 10.5 | 4.4 | 3.0 | 20.0 | 59.6 | 2.6 | | 0.2 | 0.182 |
| TX-102-1 | TFA | 16.6 | 4.3 | 3.9 | 15.4 | 50.7 | 9.1 | 1.1 | | |
| TX-102-1 | TAG | 10.7 | 4.4 | 5.3 | 21.9 | 54.1 | 3.6 | | 0.3 | 0.273 |
| TX-102-5 | TFA | 16.7 | 4.1 | 1.7 | 11.6 | 60.2 | 5.8 | 1.1 | | |
| TX-102-5 | TAG | 11.7 | 5.5 | 2.8 | 21.4 | 56.1 | 2.5 | | 0.1 | 0.118 |
| TX-102-6 | TFA | 17.8 | 3.8 | 15.9 | 17.0 | 38.8 | 6.6 | 1.5 | | |
| TX-102-6 | TAG | 19.6 | 7.0 | 29.4 | 25.4 | 13.9 | 4.7 | | 0.4 | 0.267 |
| TX-102-2 | TFA | 15.0 | 1.9 | 1.7 | 19.1 | 56.5 | 5.9 | 1.7 | | |
| TX-102-2 | TAG | 10.6 | 1.9 | 2.7 | 30.2 | 51.2 | 3.4 | | 0.4 | 0.258 |
| TX-102-7 | TFA | 15.0 | 3.1 | 7.0 | 13.9 | 56.1 | 4.9 | 2.4 | | |
| TX-102-7 | TAG | 16.1 | 6.5 | 20.5 | 28.0 | 24.4 | 4.5 | | 0.3 | 0.111 |
| TX-102-3 | TFA | 14.4 | 3.5 | 9.5 | 13.4 | 50.9 | 8.2 | 2.5 | | |
| TX-102-3 | TAG | 16.9 | 6.7 | 23.9 | 24.7 | 22.5 | 5.2 | | 0.4 | 0.150 |

TABLE 5
TFA and TAG levels, fatty acid composition and TTQ in sorghum leaves transformed with
pOIL102 (pZmUbi:WRI1) and pOIL197 (pZmUbi:DGAT and pZmUbi:Oleosin) during the
boot leaf stage of growth. The lines are listed in order of increasing TFA levels.

| Line | TAG or TFA | C16:0 | C18:0 | C18:1 | C18:2 | C18:3n3 | Other | TFA | TAG | TTQ |
|---|---|---|---|---|---|---|---|---|---|---|
| TX-02-27 | TFA | 17.3 | 3.8 | 1.4 | 10.1 | 60.1 | 7.2 | 1.0 | | |
| TX-02-27 | TAG | 11.9 | 4.4 | 2.1 | 19.4 | 61.2 | 0.8 | | 0.2 | 0.164 |
| TX-02-21 | TFA | 15.9 | 2.3 | 2.0 | 19.3 | 53.3 | 7.3 | 1.2 | | |
| TX-02-21 | TAG | 12.6 | 3.7 | 2.7 | 27.0 | 51.0 | 3.0 | | 0.4 | 0.318 |
| TX-02-01 | TFA | 15.2 | 4.2 | 5.1 | 14.7 | 53.2 | 7.5 | 1.3 | | |
| TX-02-01 | TAG | 11.7 | 5.6 | 9.3 | 26.1 | 42.9 | 4.5 | | 0.3 | 0.199 |
| TX-02-12 | TFA | 15.3 | 3.2 | 2.0 | 13.6 | 58.9 | 6.9 | 1.3 | | |
| TX-02-12 | TAG | 13.7 | 4.2 | 3.6 | 25.1 | 50.4 | 2.9 | | 0.1 | 0.111 |
| TX-02-33 | TFA | 15.9 | 4.3 | 1.0 | 10.1 | 59.7 | 9.1 | 1.4 | | |
| TX-02-33 | TAG | 14.3 | 5.4 | 2.7 | 18.9 | 54.7 | 4.0 | | 0.1 | 0.107 |
| TX-02-13 | TFA | 15.4 | 5.1 | 11.4 | 19.4 | 39.1 | 9.5 | 1.4 | | |
| TX-02-13 | TAG | 12.9 | 6.5 | 20.3 | 25.2 | 28.6 | 6.4 | | 0.5 | 0.389 |
| TX-02-36 | TFA | 16.2 | 3.4 | 1.8 | 12.3 | 58.5 | 7.8 | 1.4 | | |
| TX-02-36 | TAG | 15.4 | 5.8 | 3.3 | 21.5 | 48.9 | 5.1 | | 0.3 | 0.209 |
| TX-02-37 | TFA | 13.3 | 3.5 | 1.3 | 9.9 | 65.3 | 6.7 | 1.4 | | |
| TX-02-37 | TAG | 9.6 | 3.6 | 3.8 | 20.4 | 60.6 | 2.1 | | 0.2 | 0.137 |
| TX-02-18 | TFA | 14.6 | 3.0 | 1.4 | 9.8 | 65.5 | 5.7 | 1.4 | | |
| TX-02-18 | TAG | 12.5 | 5.6 | 4.3 | 20.6 | 54.8 | 2.3 | | 0.1 | 0.077 |
| TX-02-34 | TFA | 16.6 | 2.2 | 2.2 | 17.6 | 54.7 | 6.7 | 1.4 | | |
| TX-02-34 | TAG | 14.1 | 2.8 | 4.1 | 30.3 | 44.7 | 4.1 | | 0.3 | 0.231 |
| TX-02-31 | TFA | 13.3 | 3.1 | 1.8 | 10.1 | 64.7 | 7.0 | 1.5 | | |
| TX-02-31 | TAG | 5.4 | 1.8 | 3.2 | 17.8 | 71.1 | 0.7 | | 0.3 | 0.171 |
| TX-02-29 | TFA | 13.2 | 3.2 | 1.1 | 8.2 | 68.6 | 5.6 | 1.6 | | |
| TX-02-29 | TAG | 10.5 | 4.7 | 2.9 | 18.1 | 62.0 | 1.8 | | 0.1 | 0.082 |
| TX-02-35 | TFA | 17.8 | 3.4 | 6.5 | 14.0 | 50.3 | 8.0 | 1.6 | | |
| TX-02-35 | TAG | 18.8 | 5.3 | 19.1 | 28.4 | 22.4 | 6.1 | | 0.2 | 0.108 |
| TX-02-09 | TFA | 14.0 | 3.3 | 0.9 | 9.9 | 66.0 | 6.0 | 1.6 | | |
| TX-02-09 | TAG | 11.2 | 4.7 | 1.9 | 19.6 | 58.7 | 3.9 | | 0.1 | 0.036 |
| TX-02-24 | TFA | 12.9 | 3.5 | 0.6 | 7.9 | 67.3 | 7.7 | 1.8 | | |
| TX-02-24 | TAG | 10.7 | 3.5 | 1.6 | 11.8 | 69.0 | 3.4 | | 0.1 | 0.044 |
| TX-02-126 | TFA | 13.8 | 2.7 | 1.1 | 9.9 | 66.4 | 6.0 | 1.8 | | |
| TX-02-126 | TAG | 12.8 | 4.3 | 2.1 | 17.0 | 58.6 | 5.2 | | 0.5 | 0.247 |
| TX-02-23 | TFA | 13.6 | 2.7 | 0.7 | 8.9 | 68.3 | 5.8 | 1.9 | | |
| TX-02-23 | TAG | 10.0 | 3.3 | 2.2 | 18.2 | 63.9 | 2.4 | | 0.1 | 0.047 |
| TX-02-07 | TFA | 17.5 | 2.3 | 10.9 | 17.5 | 44.5 | 7.3 | 1.9 | | |
| TX-02-07 | TAG | 21.0 | 3.9 | 24.5 | 27.4 | 15.2 | 8.0 | | 0.4 | 0.225 |
| TX-02-28 | TFA | 12.8 | 2.9 | 0.5 | 7.7 | 68.4 | 7.8 | 2.0 | | |
| TX-02-28 | TAG | 13.0 | 5.5 | 1.2 | 11.1 | 64.3 | 4.8 | | 0.1 | 0.063 |
| TX-02-04 | TFA | 13.6 | 2.9 | 1.2 | 12.1 | 65.3 | 4.9 | 2.1 | | |
| TX-02-04 | TAG | 12.0 | 4.4 | 2.4 | 21.6 | 55.9 | 3.6 | | 0.4 | 0.206 |
| TX-02-25 | TFA | 12.2 | 2.8 | 0.5 | 9.4 | 68.8 | 6.3 | 2.5 | | |
| TX-02-25 | TAG | 10.3 | 4.2 | 1.0 | 15.4 | 62.5 | 6.6 | | 0.4 | 0.159 |
| TX-02-05 | TFA | 13.6 | 3.6 | 3.2 | 14.7 | 59.8 | 5.1 | 2.5 | | |
| TX-02-05 | TAG | 12.2 | 5.5 | 7.0 | 26.8 | 43.4 | 5.1 | | 0.6 | 0.220 |
| TX-02-14 | TFA | 15.9 | 5.7 | 30.9 | 12.7 | 26.0 | 8.9 | 2.8 | | |
| TX-02-14 | TAG | 17.9 | 8.5 | 42.6 | 14.9 | 7.8 | 8.4 | | 1.4 | 0.514 |
| TX-02-131 | TFA | 12.6 | 1.4 | 0.6 | 8.3 | 73.1 | 3.9 | 2.9 | | |
| TX-02-131 | TAG | 16.0 | 3.9 | 1.9 | 18.0 | 53.9 | 6.3 | | 0.2 | 0.061 |
| TX-02-129 | TFA | 12.1 | 1.6 | 1.0 | 10.4 | 70.5 | 4.3 | 2.9 | | |
| TX-02-129 | TAG | 12.8 | 3.6 | 2.5 | 22.0 | 53.6 | 5.5 | | 0.3 | 0.106 |
| TX-02-08 | TFA | 17.6 | 2.6 | 5.6 | 17.2 | 51.2 | 5.8 | 3.0 | | |
| TX-02-08 | TAG | 24.4 | 5.9 | 15.8 | 29.3 | 15.8 | 8.8 | | 0.6 | 0.183 |
| TX-02-02 | TFA | 17.9 | 3.1 | 7.2 | 15.5 | 49.6 | 6.7 | 3.1 | | |
| TX-02-02 | TAG | 23.7 | 6.5 | 17.7 | 22.8 | 19.6 | 9.7 | | 0.6 | 0.194 |
| TX-02-11 | TFA | 25.1 | 4.1 | 9.0 | 16.3 | 36.3 | 9.1 | 3.2 | | |
| TX-02-11 | TAG | 33.3 | 6.6 | 13.9 | 20.9 | 16.0 | 9.3 | | 1.1 | 0.341 |
| TX-02-127 | TFA | 11.4 | 1.6 | 0.3 | 8.9 | 75.4 | 2.4 | 3.5 | | |
| TX-02-127 | TAG | 21.0 | 5.8 | 1.4 | 20.6 | 47.4 | 3.9 | | 0.1 | 0.016 |
| TX-02-30 | TFA | 16.4 | 3.1 | 3.7 | 17.1 | 53.8 | 5.9 | 4.0 | | |
| TX-02-30 | TAG | 21.3 | 5.0 | 7.6 | 27.1 | 30.5 | 8.5 | | 0.9 | 0.236 |
| TX-02-19 | TFA | 13.5 | 2.7 | 25.4 | 22.6 | 30.8 | 5.0 | 4.2 | | |
| TX-02-19 | TAG | 14.0 | 3.3 | 34.3 | 27.0 | 16.6 | 4.8 | | 2.3 | 0.548 |
| TX-02-06 | TFA | 24.0 | 4.8 | 14.3 | 19.6 | 29.7 | 7.7 | 4.8 | | |
| TX-02-06 | TAG | 29.7 | 6.9 | 19.2 | 23.0 | 13.4 | 7.7 | | 2.7 | 0.555 |
| TX-02-10 | TFA | 22.0 | 3.3 | 10.3 | 22.7 | 33.7 | 7.9 | 6.3 | | |
| TX-02-10 | TAG | 24.8 | 4.1 | 12.9 | 27.0 | 22.4 | 8.8 | | 3.5 | 0.551 |
| TX-02-38 | TFA | 24.8 | 4.4 | 13.9 | 24.5 | 23.7 | 8.7 | 6.4 | | |
| TX-02-38 | TAG | 21.5 | 5.3 | 8.6 | 25.2 | 39.3 | 0.0 | | 2.5 | 0.392 |

TABLE 6
TFA and TAG levels, fatty acid composition and TTQ in pOIL103 +
pOIL197 primary transformants at boot leaf stage.

| Line | TFA or TAG | C16:0 | C18:0 | C18:1 | C18:2 | C18:3n3 | Other | TFA | TAG | TTQ |
|---|---|---|---|---|---|---|---|---|---|---|
| TX-03-20 | TFA | 12.2 | 2.6 | 1.7 | 10.3 | 67.5 | 5.7 | 2.1 | | |
| TX-03-20 | TAG | 9.4 | 3.6 | 3.3 | 18.1 | 63.0 | 2.5 | | 0.4 | 0.217 |
| TX-03-54 | TFA | 13.6 | 3.5 | 3.0 | 12.1 | 61.5 | 6.4 | 2.1 | | |
| TX-03-54 | TAG | 14.1 | 6.9 | 7.0 | 22.5 | 43.5 | 6.0 | | 0.4 | 0.207 |
| TX-03-61 | TFA | 23.9 | 3.1 | 1.7 | 19.0 | 43.9 | 8.3 | 2.2 | | |
| TX-03-61 | TAG | 31.4 | 6.6 | 3.4 | 28.3 | 19.6 | 10.8 | | 0.4 | 0.159 |
| TX-03-02 | TFA | 14.9 | 3.0 | 2.8 | 12.1 | 60.6 | 6.6 | 2.2 | | |
| TX-03-02 | TAG | 14.8 | 5.5 | 5.6 | 20.6 | 46.7 | 6.8 | | 0.5 | 0.222 |
| TX-03-53 | TFA | 18.5 | 3.7 | 8.9 | 15.4 | 43.1 | 10.4 | 2.3 | | |
| TX-03-53 | TAG | 20.1 | 6.8 | 16.7 | 24.5 | 23.3 | 8.6 | | 0.6 | 0.275 |
| TX-03-01 | TFA | 13.4 | 3.0 | 3.0 | 12.5 | 61.8 | 6.4 | 2.3 | | |
| TX-03-01 | TAG | 13.9 | 5.5 | 7.5 | 23.0 | 42.6 | 7.4 | | 0.4 | 0.164 |
| TX-03-47 | TFA | 12.8 | 2.1 | 1.6 | 7.5 | 70.7 | 5.3 | 2.4 | | |
| TX-03-47 | TAG | 14.8 | 5.1 | 5.0 | 19.3 | 52.1 | 3.7 | | 0.1 | 0.050 |
| TX-03-07 | TFA | 18.4 | 2.8 | 7.6 | 15.6 | 47.1 | 8.5 | 2.5 | | |
| TX-03-07 | TAG | 25.8 | 6.4 | 18.7 | 25.5 | 15.2 | 8.5 | | 0.3 | 0.127 |
| TX-03-05 | TFA | 21.4 | 2.3 | 1.4 | 9.7 | 59.1 | 6.1 | 2.6 | | |
| TX-03-05 | TAG | 36.4 | 5.6 | 3.9 | 17.1 | 28.4 | 8.6 | | 0.4 | 0.168 |
| TX-03-49 | TFA | 18.1 | 3.7 | 8.2 | 13.2 | 52.0 | 4.9 | 2.6 | | |
| TX-03-49 | TAG | 24.1 | 8.2 | 18.3 | 20.9 | 18.8 | 9.7 | | 0.5 | 0.212 |
| TX-03-34 | TFA | 19.0 | 2.7 | 6.0 | 15.4 | 50.6 | 6.4 | 2.6 | | |
| TX-03-34 | TAG | 24.8 | 10.5 | 10.9 | 23.9 | 20.6 | 9.3 | | 0.8 | 0.287 |
| TX-03-32 | TFA | 18.2 | 2.2 | 1.6 | 12.4 | 60.2 | 5.4 | 2.8 | | |
| TX-03-32 | TAG | 20.8 | 14.6 | 3.2 | 21.4 | 31.5 | 8.5 | | 0.6 | 0.204 |
| TX-03-04 | TFA | 18.8 | 3.1 | 5.8 | 13.4 | 50.3 | 8.6 | 2.9 | | |
| TX-03-04 | TAG | 26.7 | 7.5 | 14.6 | 23.1 | 19.0 | 9.1 | | 0.3 | 0.118 |
| TX-03-23 | TFA | 18.9 | 1.7 | 1.0 | 7.9 | 63.2 | 7.3 | 2.9 | | |
| TX-03-23 | TAG | 25.0 | 4.6 | 2.5 | 18.1 | 39.6 | 10.2 | | 0.2 | 0.070 |
| TX-03-25 | TFA | 14.5 | 1.8 | 0.4 | 6.4 | 73.5 | 3.4 | 3.0 | | |
| TX-03-25 | TAG | 20.3 | 5.1 | 1.0 | 12.3 | 53.6 | 7.7 | | 0.3 | 0.110 |
| TX-03-18 | TFA | 21.1 | 2.9 | 1.2 | 17.8 | 46.3 | 10.7 | 3.0 | | |
| TX-03-18 | TAG | 22.6 | 5.9 | 4.5 | 31.1 | 22.6 | 13.3 | | 0.4 | 0.143 |
| TX-03-50 | TFA | 16.5 | 2.6 | 6.1 | 12.9 | 53.9 | 8.0 | 3.0 | | |
| TX-03-50 | TAG | 20.2 | 19.9 | 12.9 | 19.6 | 20.6 | 6.8 | | 0.7 | 0.217 |
| TX-03-60 | TFA | 20.2 | 2.9 | 0.8 | 14.1 | 55.7 | 6.2 | 3.1 | | |
| TX-03-60 | TAG | 30.5 | 6.2 | 1.6 | 21.6 | 30.2 | 9.9 | | 0.6 | 0.202 |
| TX-03-21 | TFA | 12.3 | 1.7 | 0.5 | 6.8 | 74.4 | 4.4 | 3.2 | | |
| TX-03-21 | TAG | 16.1 | 4.7 | 1.6 | 13.1 | 57.0 | 7.5 | | 0.2 | 0.067 |
| TX-03-40 | TFA | 17.1 | 1.4 | 0.4 | 8.0 | 68.2 | 4.9 | 3.2 | | |
| TX-03-40 | TAG | 34.5 | 4.4 | 0.9 | 14.5 | 39.8 | 5.9 | | 0.4 | 0.112 |
| TX-03-62 | TFA | 25.3 | 2.9 | 1.7 | 14.7 | 47.9 | 7.6 | 3.3 | | |
| TX-03-62 | TAG | 40.3 | 5.6 | 3.5 | 22.3 | 18.7 | 9.5 | | 0.6 | 0.171 |
| TX-03-36 | TFA | 19.5 | 2.0 | 2.0 | 11.4 | 58.3 | 6.8 | 3.5 | | |
| TX-03-36 | TAG | 31.2 | 4.0 | 4.4 | 20.0 | 29.4 | 11.0 | | 0.6 | 0.160 |
| TX-03-63 | TFA | 25.4 | 3.6 | 2.6 | 18.2 | 42.0 | 8.2 | 3.5 | | |
| TX-03-63 | TAG | 33.1 | 6.1 | 3.8 | 24.9 | 21.6 | 10.4 | | 1.4 | 0.383 |
| TX-03-45 | TFA | 16.4 | 1.4 | 0.5 | 8.1 | 69.1 | 4.5 | 3.5 | | |
| TX-03-45 | TAG | 30.8 | 4.6 | 1.4 | 16.2 | 40.7 | 6.3 | | 0.2 | 0.058 |
| TX-03-17 | TFA | 14.2 | 1.8 | 0.8 | 6.9 | 71.2 | 5.2 | 3.6 | | |
| TX-03-17 | TAG | 18.7 | 4.5 | 2.2 | 13.5 | 52.8 | 8.3 | | 0.4 | 0.120 |
| TX-03-57 | TFA | 18.7 | 3.4 | 1.5 | 13.8 | 55.8 | 6.8 | 3.6 | | |
| TX-03-57 | TAG | 23.4 | 6.3 | 3.0 | 21.0 | 36.2 | 10.1 | | 1.2 | 0.330 |
| TX-03-11 | TFA | 29.1 | 6.4 | 2.1 | 22.4 | 33.0 | 7.1 | 3.6 | | |
| TX-03-11 | TAG | 30.6 | 8.5 | 2.8 | 27.0 | 19.7 | 11.4 | | 1.9 | 0.510 |
| TX-03-48 | TFA | 27.1 | 3.7 | 3.7 | 20.6 | 37.2 | 7.6 | 3.7 | | |
| TX-03-48 | TAG | 31.2 | 5.0 | 5.5 | 27.1 | 23.0 | 8.1 | | 2.1 | 0.569 |
| TX-03-29 | TFA | 20.1 | 2.3 | 1.7 | 13.4 | 55.5 | 7.1 | 3.7 | | |
| TX-03-29 | TAG | 33.0 | 5.0 | 4.1 | 24.3 | 26.4 | 7.2 | | 0.4 | 0.104 |
| TX-03-26 | TFA | 15.3 | 1.6 | 0.4 | 5.9 | 71.3 | 5.5 | 3.9 | | |
| TX-03-26 | TAG | 25.2 | 4.6 | 1.7 | 13.3 | 49.7 | 5.5 | | 0.3 | 0.074 |
| TX-03-10 | TFA | 28.6 | 6.8 | 2.1 | 21.8 | 33.0 | 7.7 | 3.9 | | |
| TX-03-10 | TAG | 31.0 | 8.5 | 2.9 | 26.7 | 18.6 | 12.2 | | 1.9 | 0.491 |
| TX-03-58 | TFA | 16.3 | 2.6 | 1.3 | 14.5 | 60.3 | 5.0 | 4.1 | | |
| TX-03-58 | TAG | 20.4 | 5.2 | 2.8 | 24.3 | 39.2 | 8.2 | | 1.1 | 0.278 |
| TX-03-08 | TFA | 19.8 | 2.0 | 0.7 | 6.6 | 64.9 | 5.9 | 4.1 | | |
| TX-03-08 | TAG | 34.8 | 5.2 | 2.7 | 14.3 | 34.5 | 8.5 | | 0.2 | 0.051 |
| TX-03-33 | TFA | 27.4 | 2.4 | 1.5 | 16.3 | 46.0 | 6.4 | 4.2 | | |
| TX-03-33 | TAG | 39.2 | 5.4 | 2.3 | 21.9 | 20.8 | 10.5 | | 1.6 | 0.386 |
| TX-03-22 | TFA | 19.8 | 2.8 | 3.1 | 11.8 | 53.4 | 9.1 | 4.2 | | |
| TX-03-22 | TAG | 28.4 | 5.3 | 5.4 | 19.4 | 38.3 | 3.2 | | 1.2 | 0.287 |
| TX-03-41 | TFA | 18.1 | 2.6 | 3.1 | 11.1 | 58.0 | 7.1 | 4.8 | | |
| TX-03-41 | TAG | 27.8 | 6.0 | 6.8 | 19.3 | 34.9 | 5.3 | | 0.7 | 0.139 |
| TX-03-46 | TFA | 24.6 | 2.0 | 0.6 | 7.9 | 57.4 | 7.4 | 4.9 | | |
| TX-03-46 | TAG | 44.7 | 4.2 | 1.3 | 13.4 | 31.4 | 5.0 | | 1.1 | 0.220 |
| TX-03-28 | TFA | 28.5 | 2.1 | 1.3 | 23.4 | 33.7 | 11.0 | 6.2 | | |
| TX-03-28 | TAG | 36.0 | 2.9 | 3.1 | 29.6 | 18.5 | 10.0 | | 3.7 | 0.596 |
| TX-03-31 | TFA | 33.4 | 2.9 | 4.3 | 28.6 | 25.5 | 5.5 | 8.3 | | |
| TX-03-31 | TAG | 38.0 | 3.6 | 4.9 | 30.6 | 14.8 | 8.1 | | 6.6 | 0.789 |

### Example 3. Increasing Expression of Thioesterase in Plant Cells

De novo fatty acid synthesis takes place in the plastids of eukaryotic cells where the fatty acids are synthesized while bound to acyl carrier protein as acyl-ACP conjugates. Following chain elongation to C16:0 and C18:0 acyl groups and then desaturation to C18:1 while linked to ACP, the fatty acids are cleaved from the ACP by thioesterases and enter the eukaryotic pathway by export from the plastids and transport to the ER where they participate in membrane and storage lipid biogenesis. In chloroplasts, the export process has two steps: firstly, acyl chains are released as free fatty acids by the enzymatic activity of acyl-ACP thioesterases (fatty acyl thioesterase; FAT), secondly by reaction with CoA to form acyl-CoA esters which is catalysed by long chain acyl-CoA synthetases (LACS). A. thaliana contains 3 fatty acyl thioesterases which can be distinguished based on their acyl chain specificity. FATA1 and FATA2 preferentially hydrolyze unsaturated acyl-ACPs while saturated acyl-ACP chains are typically cleaved by FATB.

To explore the effect upon total fatty acid content, TAG content, and fatty acid composition of the co-expression of a thioesterase and genes encoding the WRI1 and/or DGAT polypeptides, chimeric genes were made for each of the three A. thaliana thioesterases by insertion of the coding regions into the pJP3343 binary expression vector for transient expression in N. benthamiana leaf cells from the 35S promoter. Protein coding regions for the A. thaliana FATAL (Accession No. NP_189147.1, SEQ ID NO:43) and FATA2 (Accession No. NP_193041.1, SEQ ID NO:44) thioesterases were amplified from silique cDNA using primers containing EcoRI and PstI sites and subsequently cloned into pJP3343 using the same restriction sites. The resulting expression vectors were designated pOIL079 and pOIL080, respectively. The protein coding region of the A. thaliana FATB gene (Accession No. NP_172327.1, SEQ ID NO:45) was amplified using primers containing NotI and SacI flanking sites and cloned into the corresponding restriction sites of pJP3343, resulting in pOIL081. Constructs pOIL079, pOIL080 and pOIL081 are infiltrated into N. benthamiana leaf tissue, either individually or in combination with constructs containing the genes for the A. thaliana WRI1 transcription factor (AtWRI1) (pJP3414) and/or DGAT1 acyltransferase (AtDGAT1) (pJP3352). For comparison, chimeric genes encoding the Cocos nucifera FatB1 (CnFATB1) (pJP3630), C. nucifera FatB2 (CnFATB2) (pJP3629) were introduced into N. benthamiana leaf tissue in parallel with the Arabidopsis thioesterases, to compare the effect of the FatB polypeptides having MCFA specificity to the Arabidopsis thioesterases which do not have MCFA specificity. All of the infiltrations included a chimeric gene for expression of the p19 silencing suppressor as described in Example 1. The negative control infiltrated only the p19 T-DNA.

A synergistic effect was observed between thioesterase expression and WRI1 and/or DGAT over-expression on TAG levels in N. benthamiana leaves. Expression of the thioesterase genes without the WRI1 or DGAT genes significantly increased TAG levels above the low level in the negative control (p19 alone). For example, expression of the coconut FATB2 thioesterase resulted in an 8.2-fold increase in TAG levels in the leaves compared to the negative control. Co-expression of the A. thaliana WRI1 transcription factor with each of the thioesterases further increased TAG levels compared to the AtWRI1 control. Co-expression of each of the coconut thioesterases CnFATB1 and CnFATB2 with WRI1 resulted in higher TAG levels than each of the three A. thaliana thioesterases with WRI1. Interestingly, the converse was observed when the A. thaliana DGAT1 acyltransferase was co-expressed in combination with a thioesterase and WRI1. This suggested a better match in acyl-chain specificity of the A. thaliana thioesterases and the A. thaliana DGAT1 acyltransferase, resulting in a greater flux of acyl-chains from the acyl-ACP into TAG. The non-MCFA thioesterases were also considerably more effective in elevating the percentage of oleic acid in the total fatty acid content in the leaves. Co-expression of the AtWRI1, AtDGAT1 and AtFATA2 resulted in the greatest level of TAG in the leaves, providing a level which was 1.6-fold greater than when AtWRI1 and AtDGAT1 were co-expressed without the thioesterase. In another experiment, transient overexpression of FATA2 in combination with WRI1 and DGAT1 led to a 2.5-fold increase in TAG level relative to a p19+WRI1+DGAT1 control, which represented a 50-fold increase in TAG levels relative to p19 alone. Addition of FATA1 increased TAG levels 2-fold compared to p19+WRI1+DGAT1, a 40-fold increase compared to p19 alone. Addition of FATB increased TAG levels by 1.6-fold relative to p19+WRI1+DGAT1, a 32-fold increase relative to p19 control.

Co-expression of thioesterase FATA or FATB together with WRI1 and DGAT1 resulted in modified leaf fatty acid composition relative to WRI1 and DGAT1 without thioesterase. Addition of FATAL increased the percentages of C16:0 and C18:0 at the expense of saturated fatty acids. Addition of FATA2 also increased the proportion of C18:0 but did not have as great an effect on C16:0. In contrast, addition of FATB increased C16:0 but not C18:0 levels. In each case, addition of FATA1, FATA2 and FATB reduced C18:1 levels. Notably, the C16:0 percentage increased from 28.4% in p19+WRI1+DGAT1 without thioesterase to 43.8% with the addition of FATA1, to 34.4% with the addition of FATA2 and to 46.3% with the addition of FATB.

These experiments confirmed the synergistic increase in oil synthesis and accumulation when both WRI1 and DGAT were co-expressed as well as showing the further synergistic increase obtained by adding a thioesterase to the combination.

### Effect of Transient Thioesterase Expression in a High Oil Background

The three A. thaliana thioesterase genes were also tested by transient expression in leaves of N. benthamiana plants (transgenic line AT001) which were transgenic for and stably expressing WRI1, DGAT1 and OLEOSIN genes (El Tahchy et al., 2017). Thirty plants from homozygous, T2 generation, transgenic AT001 seeds were grown in a randomised design alongside wild-type (WT) controls. At a vegetative stage of growth, 53 days after sowing (DAS), the transgenic leaves contained about 8.7% (DW) TAG compared to about 0.03% (DW) TAG in the wild-type plants. After further growth of the transgenic plants, TAG levels increased from about 11.2% to about 21.3% (DW) during flowering stages. They continued to increase, reaching about 31.4% (DW) TAG at maturity (late seed development stage). As the plants senesced, the TAG level in at least some plants decreased to about 19.6% DW.

The genes encoding the thioesterases were introduced into leaves of young plants (49 DAS) when the leaves typically had about 3.1% (DW) TAG, and sampled 5 days after infiltration with the Agrobacterium strains. Leaf samples were harvested and analyzed for TAG content. FATA2 overexpression in AT001 N. benthamiana leaves significantly increased TAG to 4.4% (DW) compared to the p19 control (3.1% TAG). FATA1 increased TAG content to 3.9% (DW). FATB transient expression did not appear to increase TAG accumulation in this experiment.

Samples were also used in radiolabel feeding assays with [¹⁴C]-acetate. [¹⁴C]-acetate was added in a 10 minute pulse to leaf discs of AT001 leaves, infiltrated previously with genes encoding p19 and one of FATA1, FATA2 and FATB. This pulse was followed by a 20 minute chase. Lipid extracts were prepared at each time point followed by separation of labelled lipid classes on TLC. Quantitation of the labelled reaction products showed increases in the rate of TAG production in the AT001 leaves transiently expressing FATA1 (602 DPM), FATA2 (762 DPM) and FATB (559 DPM) compared to the p19 control (283 DPM).

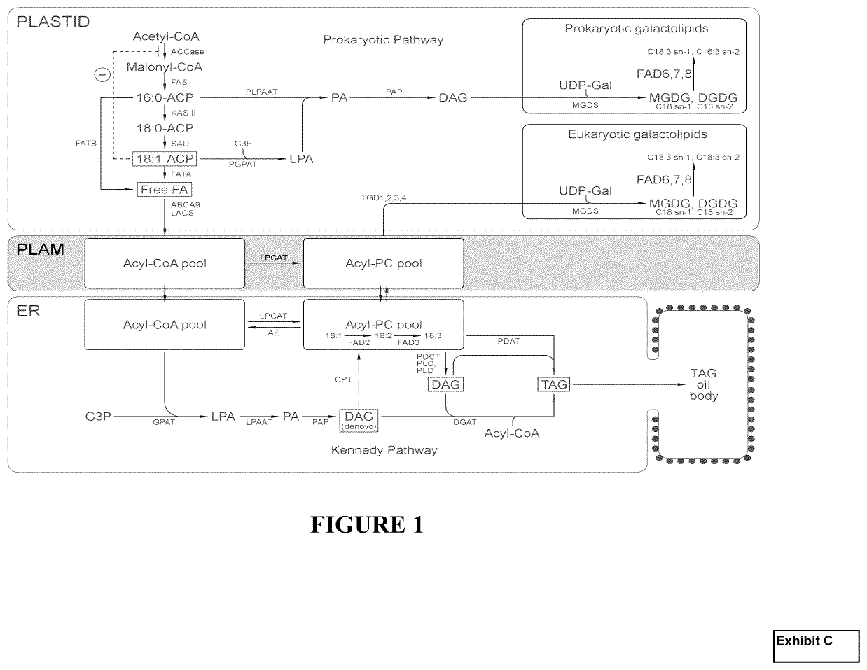

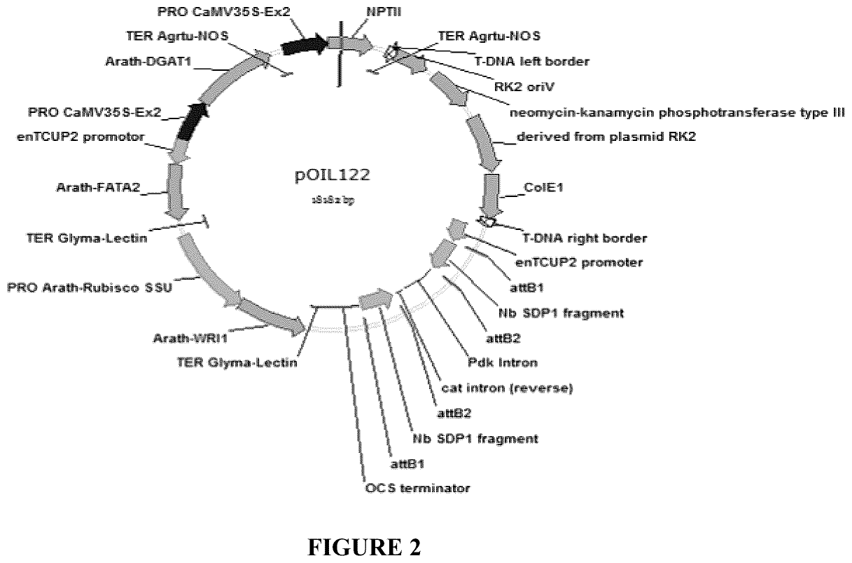

Three different binary expression vectors were constructed to test the effect of co-expression of genes encoding WRI1, DGAT1 and FATA on TAG levels and fatty acid composition in stably transformed N. tabacum leaves. The vector pOIL 121 contained an SSU::AtWRI1 gene for expression of AtWRI1 from the SSU promoter, a 35S::AIDGAT1 gene for expression of AtDGAT from the 35S promoter, and an enTCUP2::AtFATA2 gene for expression of AtFATA2 from the enTCUP2 promoter which is a constitutive promoter. These genetic constructs were derived from pOIL38 by first digesting the DNA with NotI to remove the gene coding for the S. indicum oleosin. The protein coding region of the A. thaliana FATA2 gene was amplified and flanked with NotI sites using pOIL80 DNA as template. This fragment was then inserted into the NotI site of pOIL38. pOIL121 then served as a parent vector for pOIL 122 which contained an additional enTCUP2::SDP1 hairpin RNA cassette for RNAi-mediated silencing of the endogenous SDP1 gene in the transgenic plants. To do this, the entire N. benthamiana SDP1 hairpin cassette was isolated from pOIL51 (Vanhercke et al., 2017) as an SfoI-SmaI fragment and cloned into the SfoI site of pOIL 121, producing pOIL 122 (FIG. 2). A third vector, pOIL 123, containing the SSU::WRI1 and 35S::DGAT1 genes and the enTCUP2::SDP1 hairpin RNA gene was obtained in a similar way by cloning the enTCUP2::SDP1 hairpin RNA cassette as a SfoI-SmaI fragment into the SfoI site of pOIL36.

In summary, the vectors contained the gene combinations:
pOIL 121: SSU::AtWRI1, 35S::AtDGAT1, enTCUP2::AtFATA2.
pOIL 122: SSU::AtWRI1, 35S::AtDGAT1, enTCUP2::AtFATA2, enTCUP2::SDP1 hairpin.
pOIL 123: SSU::AtWRI1, 35S::AtDGAT1, enTCUP2::SDP1 hairpin.

The three constructs were each used to produce transformed N. tabacum plants (cultivar Wi38) by Agrobacterium-mediated transformation. Co-expression of the A. thaliana FATA2 thioesterase or silencing of the endogenous SDP1 TAG lipase in combination with AtWRI1 and AtDGAT1 expression each resulted in further elevated TAG levels compared to expression of AtWRI1 and AtDGAT1 in the absence of both of the thioesterase gene and the SDP1-silencing gene. The greatest TAG yields were obtained using pOIL 122 by the combined action of all four chimeric genes. In absence of SDP1, pOIL121 lines yielded 13.3% TAG which was included increased palmitate (16:0) levels (36%) and reduced ALA (18:303) levels (7%).

It is noted that N. benthamiana is an 18:3 plant. The same constructs pOIL079, pOIL080 and pOIL081 are used to transform A. thaliana, a 16:3 plant.

The inventors conceived of the model that increasing plastidial fatty acid export such as by increased fatty acyl thioesterase activity reduces acyl-ACP accumulation in the plastids, thereby increasing fatty acid biosynthesis as a result of reduced feedback inhibition on the acetyl-CoA carboxylase (ACCase) (Andre et al., 2012; Moreno-Perez et al., 2012). Thioesterase over-expression increases export of acyl chains from the plastids into the ER, thereby providing an efficient link between so-called ‘Push’ and ‘Pull’ metabolic engineering strategies.

### Example 4. The Effect of Different Transcription Factor Polypeptides on Plant Traits

Previously reported experiments with WRI1 and DGAT (Vanhercke et al., 2013) used a synthetic gene encoding A. thaliana AtWRI1 (Accession No. AAP80382.1) and a synthetic gene encoding AtDGAT1, also from A. thaliana (Accession No. AAF19262; SEQ ID NO:1). To compare other WRI polypeptides with AtWRI1 for their ability to combine with DGAT to increase oil content, other WRI coding sequences were identified and used to generate constructs for expression in N. benthamiana leaves. Nucleotide sequences encoding the A. thaliana WRI3 (Accession No. AAM91814.1, SEQ ID NO:46) and WRI4 (Accession No. NP_178088.2, SEQ ID NO:47) transcription factors (To et al., 2012) were synthesized and inserted as EcoRI fragments into pJP3343 under the control of the 35S promoter. The resulting binary expression vectors were designated pOIL027 and pOIL028, respectively. The coding sequence for the oat (Avena sativa) WRI1 (AsWRI1, SEQ ID NO:48) was PCR amplified from a vector provided by Prof. Sten Stymne (Swedish University of Agricultural Sciences) using flanking primers containing additional EcoRI sites. The amplified fragment was inserted into pJP3343 resulting in pOIL055. A WRI1 candidate sequence from S. bicolor (Accession No. XP_002450194.1, SEQ ID NO:49) was identified by a BLASTp search on the NCBI server using the Zea mays WRI1 amino acid sequence (Accession No. NP_001137064.1, SEQ ID NO:50) as query. The protein coding region of the S. bicolor WRI1 gene (SbWRI1) was synthesized and inserted as an EcoRI fragment into pJP3343, yielding pOIL056. A gene candidate encoding a WRI1 was identified from the Chinese tallow (Triadica sebifera; TsWRI1, SEQ ID NO:51) transcriptome (Uday et al., submitted). The protein coding region was synthesized and inserted as an EcoRI fragment into pJP3343 resulting in pOIL070. The pJP3414 and pJP3352 binary vectors containing the coding sequences for expression of the A. thaliana WRI1 and DGAT1 polypeptides were as described by Vanhercke et al. (2013).

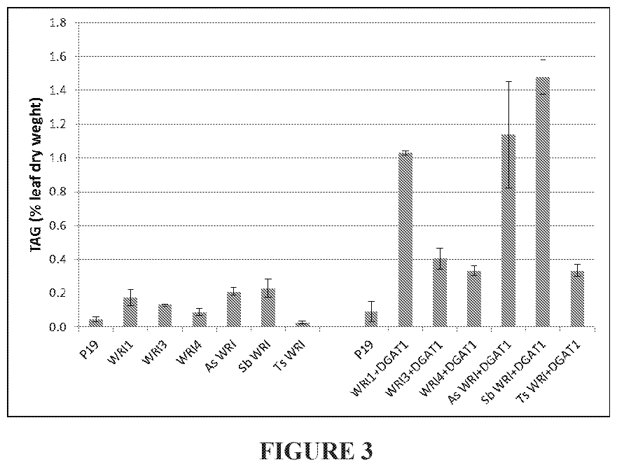

Plasmids containing the various WRI coding sequences were introduced into N. benthamiana leaf tissue for transient expression using a gene encoding the p19 viral suppressor protein in all inoculations as described in Example 1. The genes encoding the WRI polypeptides were either tested alone or in combination with the DGAT1 acyltransferase gene, the latter to provide greater TAG biosynthesis and accumulation. The positive control in this experiment was the combination of the genes encoding A. thaliana WRI1 transcription factor and AtDGAT1. All infiltrations were done in triplicate using three different plants and TAG levels were analyzed as described in Example 1. Expression of most of the individual WRI polypeptides in the absence of exogenously added DGAT1 resulted in increased, yet still low, TAG levels (<0.23% on dry weight basis) in infiltrated leaf spots, compared to the control which had only the p19 construct (FIG. 3). The exception was TsWRI1 which, by itself, did not appear to increase TAG levels significantly. In addition, differences in TAG levels produced by expression of the different WRI transcription factors on their own were not great. Both AsWRI1 and SbWRI1 yielded TAG levels similar to AtWRI1 on its own. Analysis of the TAG fatty acid composition revealed only minor changes except for increased C18:149 levels from expression of AtWRI3 in the infiltrated leaf tissues (Table 7).

In contrast, differences in0 TAG yields from expression of the different WRI polypeptides were more pronounced upon co-expression with the AtDGAT1 acyltransferase. This again demonstrated the synergistic effect of WRI1 and DGAT co-expression on TAG biosynthesis in infiltrated N. benthamiana leaf tissue, as reported by Vanhercke et al. (2013). Intermediate TAG levels were observed upon co-expression of DGAT1 with AtWRI3, AtWRI4 and TsWRI1 expressing vectors while levels obtained with the AsWRI1 and AtWRI1 were significantly lower. In a result that could not have been predicted beforehand, the highest TAG yields were obtained with co-expression of DGAT with SbWRI1, even though the assay was done in dicotyledonous cells. TAG fatty acid composition analysis revealed increased levels of C18:1^{Δ9} and decreased levels of C18:3^{Δ9,12,15} (ALA) in the case of SbWRI1, AsWRI1 and the AtWRI1 positive control. Unlike AtWRI1, however, expression of AsWRI1 and SbWRI1 both displayed increased C16:0 levels compared to the p19 negative control. Interestingly, AtWRI3 infiltrated leaf samples exhibited a distinct TAG profile with C18:1.19 being enriched while C16:0 and ALA were only slightly affected.

This experiment showed that the S. bicolor WRI1 transcription factor, SbWRI1, was superior to AtWRI1 when co-expressed with DGAT to increase TAG levels in vegetative plant parts. The inventors also concluded that a transcription factor, for example a WRI1, from a monocotyledonous plant could function well in a dicotyledonous plant cell, indeed might even have superior activity compared to a corresponding transcription factor from a dicotyledonous plant. Likewise, a transcription factor from a dicotyledonous plant could function well in a monocotyledonous plant cell.

TABLE 7
TAG fatty acid composition in N. benthamiana leaf samples infiltrated with different chimeric genes for expression
of WRI (n = 3). All samples were also infiltrated with the P19 construct. The TAG samples also contained
0.1-0.4% C14:0; 0.5-1.2% C16:3 and; 0.1-0.7% C18:1Δ11.

| Infiltrated | | | | | | | | | | |
|---|---|---|---|---|---|---|---|---|---|---|
| genes | C16:0 | C16:1 | C18:0 | C18:1 | C18:2 | C18:3n3 | C20:0 | C20:1 | C22:0 | C24:0 |
| Control (P19) | 33.6 ± 4.7 | 0.5 ± 0.4 | 8.9 ± 2.2 | 4.7 ± 0.6 | 16.9 ± 1.0 | 32.2 ± 7.8 | 1.1 ± 0.2 | 0.8 ± 1.5 | 0.0 | 0.0 |
| WRI1 | 35.5 ± 3.4 | 0.7 ± 0.2 | 5.2 ± 0.8 | 5.4 ± 1.3 | 17.1 ± 1.0 | 33.1 ± 2.7 | 0.8 ± 0.1 | 0.5 ± 0.6 | 0.3 ± 0.0 | 0.0 |
| WRI3 | 27.3 ± 1.6 | 0.9 ± 0.2 | 4.8 ± 0.3 | 10.2 ± 1.5 | 16.1 ± 1.0 | 37.8 ± 1.2 | 0.8 ± 0.1 | 0.6 ± 0.7 | 0.1 ± 0.2 | 0.0 |
| WRI4 | 30.1 ± 0.4 | 1.0 ± 0.4 | 5.2 ± 0.8 | 4.6 ± 0.6 | 17.2 ± 0.4 | 38.1 ± 1.6 | 0.8 ± 0.1 | 1.3 ± 1.3 | 0.0 | 0.0 |
| AsWRI | 35.7 ± 3.0 | 1.7 ± 0.4 | 5.3 ± 0.7 | 6.5 ± 0.3 | 15.4 ± 0.4 | 31.6 ± 1.6 | 0.8 ± 0.1 | 0.4 ± 0.7 | 0.3 ± 0.1 | 0.0 |
| SbWRI | 37.4 ± 0.8 | 1.9 ± 0.3 | 4.8 ± 0.3 | 7.0 ± 1.2 | 15.2 ± 0.3 | 30.8 ± 0.3 | 0.8 ± 0.1 | 0.4 ± 0.6 | 0.3 ± 0.0 | 0.0 |
| TsWRI | 34.5 ± 4.8 | 0.0 | 9.4 ± 8.2 | 5.9 ± 1.7 | 16.0 ± 0.7 | 29.3 ± 12.4 | 0.0 | n.d. | 0.0 | 0.0 |
| Control (P19) | 31.0 ± 2.1 | 0.9 ± 0.1 | 8.7 ± 1.3 | 8.0 ± 2.3 | 24.9 ± 1.5 | 22.1 ± 4.7 | 2.0 ± 0.1 | 0.0 | 0.6 ± 0.6 | 0.2 ± 0.4 |
| WRI1 + DGAT | 27.7 ± 0.1 | 0.3 ± 0.0 | 7.0 ± 0.1 | 17.2 ± 0.7 | 27.9 ± 0.9 | 14.7 ± 0.3 | 2.4 ± 0.2 | 0.3 ± 0.0 | 1.1 ± 0.1 | 0.8 ± 0.2 |
| WRI3 + DGAT | 30.0 ± 0.8 | 0.6 ± 0.1 | 5.9 ± 0.4 | 13.9 ± 2.9 | 21.5 ± 1.1 | 21.3 ± 0.8 | 2.8 ± 0.1 | 0.2 ± 0.0 | 1.8 ± 0.1 | 1.0 ± 0.2 |
| WRI4 + DGAT | 27.0 ± 0.5 | 0.2 ± 0.1 | 8.5 ± 0.2 | 5.8 ± 0.7 | 23.9 ± 0.8 | 25.2 ± 1.3 | 3.5 ± 0.1 | 0.2 ± 0.0 | 2.1 ± 0.2 | 1.7 ± 0.2 |
| AsWRI + DGAT | 33.8 ± 0.5 | 1.1 ± 0.1 | 5.5 ± 0.9 | 12.2 ± 1.6 | 26.0 ± 1.9 | 16.3 ± 1.3 | 2.2 ± 0.2 | 0.2 ± 0.0 | 1.2 ± 0.1 | 0.8 ± 0.1 |
| SbWRI + DGAT | 34.6 ± 0.5 | 1.3 ± 0.1 | 5.6 ± 0.4 | 13.9 ± 1.6 | 23.6 ± 1.3 | 15.8 ± 0.6 | 2.2 ± 0.1 | 0.2 ± 0.0 | 1.2 ± 0.1 | 0.9 ± 0.1 |
| TsWRI + DGAT | 25.4 ± 0.5 | 0.2 ± 0.0 | 9.4 ± 0.1 | 7.7 ± 1.0 | 27.0 ± 1.3 | 22.1 ± 2.4 | 3.6 ± 0.2 | 0.2 ± 0.0 | 1.8 ± 0.2 | 1.3 ± 0.2 |

### Use of Other Transcription Factors

Genetic constructs were prepared for expression of each of 24 different transcription factors in plant cells to test their ability to function for increasing TAG levels in combination with other genes involved in TAG biosynthesis and accumulation. These transcription factors were candidates as alternatives for WRI1 or for addition to combinations including one or more of WRI1, LEC1 and LEC2 transcription factors for use in plant cells, particularly in vegetative plant parts. Their selection was largely based on their reported involvement in embryogenesis (reviewed in Baud and Lepiniec (2010), and Ikeda et al. (2006)), similar to LEC2, or plant storage lipid metabolism. Experiments were therefore carried out to assay their function, using the N. benthamiana expression system (Example 1), as follows.

Nucleotide sequences of the protein coding regions of the following transcription factors were codon optimized for expression in N. benthamiana and N. tabacum, synthesized and subcloned as NotI-SacI fragments into the respective sites of pJP3343: A. thaliana FUS3 (pOIL 164) (Luerssen et al., 1998; Accession number AAC35247; SEQ ID NO:34), A. thaliana LEC1L (pOIL165) (Kwong et al. 2003; Accession number AAN15924; SEQ ID NO:33), A. thaliana LEC1 (pOIL 166) (Lotan et al., 1998; Accession number AAC39488; SEQ ID NO:31), G. max MYB73 (pOIL167) (Liu et al., 2014; Accession number ABH02868; SEQ ID NO:57), A. thaliana bZIP53 (pOIL 168) (Alonso et al., 2009; Accession number AAM14360; SEQ ID NO:58), A. thaliana AGL15 (pOIL169) (Zheng et al., 2009; Accession number NP_196883; SEQ ID NO:59), A. thaliana MYB118 (Accession number AAS58517; pOIL170; SEQ ID NO:60), MYB115 (Wang et al., 2002; Accession number AAS10103; pOIL171; SEQ ID NO:61), A. thaliana TANMEI (pOIL 172) (Yamagishi et al., 2005; Accession number BAE44475; SEQ ID NO:62), A. thaliana WUS (pOIL173) (Laux et al., 1996; Accession number NP_565429; SEQ ID NO:63), A. thaliana BBM (pOIL 174) (Boutilier et al., 2002; Accession number AAM33893, SEQ ID NO:64), B. napus GFR2a1 (Accession number AFB74090; pOIL 177; SEQ ID NO:64), GFR2a2 (Accession number AFB74089; pOIL 178; SEQ ID NO:65) (Liu et al. (2012)), E. guineensis NF-YB1 (pOIL405) (Geurin et al., 2016; Accession number XM_010907896; SEQ ID NO:143, E. guineensis ZFP1 (pOIL406) (Geurin et al., 2016; Accession number XM_010930940; SEQ ID NO:144), A. thaliana NF-YB2 (pOIL407) (Geurin et al., 2016; Accession number NM_124138; SEQ ID NO:145), A. thaliana NF-YB3 (pOIL408) (Geurin et al., 2016; Accession number NM_117534; SEQ ID NO:146), A. thaliana ZFP2 (pOIL409) (Geurin et al, 2016; Accession number NM_125133; SEQ ID NO:147), E. guineensis ABI5 (pOIL410) (Yeap et al., 2017; Accession number XM_010909282; SEQ ID NO:148), E. guineensis NF-YC2 (pOIL411) (Yeap et al., 2017; Accession number XM_010911913; SEQ ID NO:149), and E. guineensis NF-YA3 (pOILΔ12) (Yeap et al., 2017; Accession number XM_010941630; SEQ ID NO:150). In addition, a codon optimized version of the A. thaliana PHR 1 transcription factor involved in adaptation to high light phosphate starvation conditions was similarly subcloned into pJP3343 (pOIL189) (Nilsson et al (2012); Accession number AAN72198; SEQ ID NO:221). The sequence coding for the G. max DOF4 (Wang et al., 2007; Accession number DQ857254; SEQ ID NO:151) was codon optimized for expression in N. benthamiana and N. tabacum, synthesized as a NotI-SpeI fragment and subcloned into pJP3343. The resulting vector was designated pOIL379. Finally, the gene coding for the G. max ZF351 transcription factor (Li et al., 2017; Accession number XM_003526219; SEQ ID NO:152) was synthesized as a NotI-EcoRI fragment and cloned into pJP3343, resulting in pOIL420. These transcription factors are summarised in Table 8.

As a screening assay to determine the function of these transcription factors, the genetic constructs and a gene encoding DGAT1 were co-infiltrated into N. benthamiana leaf cells as described in Example 1, either with or without a gene encoding WRI1. Total lipid content and fatty acid composition of the leaf cells were analysed 5 days post-infiltration. Among the various embryogenic transcription factors tested, only overexpression of FUS3 resulted in significantly increased TAG levels in N. benthamiana leaf tissue when compared to DGAT and DGAT1+WRI1 control infiltrations (Table 9).

TABLE 8
Additional transcription factors and the
genetic constructs for their expression

| | | | Length | |
|---|---|---|---|---|
| | Transcription | | (amino | Accession |
| Plasmid | factor | Species | acid) | number |
| pOIL164 | FUS3 | A. thaliana | 312 | AAC35247 |
| pOIL165 | LEC1L | A. thaliana | 234 | AAN15924 |
| pOIL166 | LEC1 | A. thaliana | 208 | AAC39488 |
| pOIL167 | MYB73 | G. max | 74 | ABH02868 |
| pOIL168 | bZIP53 | A. thaliana | 146 | AAM14360 |
| pOIL169 | AGL15 | A. thaliana | 268 | NP_196883 |
| pOIL170 | MYB118 | A. thaliana | 437 | AAS58517 |
| pOIL171 | MYB115 | A. thaliana | 359 | AAS10103 |
| pOIL172 | TANMEI | A. thaliana | 386 | BAE44475 |
| pOIL173 | WUS | A. thaliana | 292 | NP_565429 |
| pOIL174 | BBM | A. thaliana | 584 | AAM33803 |
| pOIL177 | GFR2a1 | B. napus | 453 | AFB74090 |
| pOIL178 | GFR2a2 | B. napus | 461 | AFB74089 |
| pOIL189 | PHR1 | A. thaliana | 409 | AAN72198 |
| pOIL379 | DOF4 | G. max | 300 | DQ857254 |
| pOIL405 | NF-YB1 | E. guineensis | 215 | XM_010907896 |
| pOIL406 | ZFP1 | E. guineensis | 142 | XM_010930940 |
| pOIL407 | NF-YB2 | A. thaliana | 190 | NM_124138 |
| pOIL408 | NF-YB3 | A. thaliana | 161 | NM_117534 |
| pOIL409 | ZFP2 | A. thaliana | 150 | NM_125133 |
| pOIL410 | ABI5 | E. guineensis | 398 | XM_010909282 |
| pOIL411 | NF-YC2 | E. guineensis | 272 | XM_010911913 |
| pOIL412 | NF-YA3 | E. guineensis | 352 | XM_010941630 |
| pOIL420 | ZF351 | G. max | 351 | 003526219 |

TABLE 9
TAG level (% leaf dry weight) and fatty acid profile of infiltrated N. benthamiana leaves.

| | C16:0 | C16:1 | C18:0 | C18:1 | C18:2 | C18:3 | TAG |
|---|---|---|---|---|---|---|---|
| P19 | 27.1 ± 1.5 | 0.3 ± 0.1 | 9.6 ± 1.7 | 4.4 ± 1.2 | 22.4 ± 4.0 | 30.5 ± 0.9 | 0.0 |
| P19 + DGAT1 | 26.3 ± 1.0 | 0.1 ± 0.0 | 10.7 ± 0.6 | 3.7 ± 0.7 | 26.1 ± 1.6 | 26.4 ± 1.4 | 0.2 ± 0.0 |
| P19 + DGAT1 + FUS3 | 24.1 ± 1.0 | 0.1 ± 0.0 | 6.3 ± 0.4 | 5.2 ± 1.6 | 27.9 ± 1.8 | 30.0 ± 1.8 | 0.6 ± 0.1 |
| P19 + DGAT1 + LEC1L | 26.0 ± 1.4 | 0.1 ± 0.0 | 10.3 ± 0.8 | 3.9 ± 1.0 | 26.6 ± 2.1 | 26.4 ± 0.7 | 0.2 ± 0.0 |
| P19 | 30.3 ± 0.7 | 0.0 | 12.4 ± 0.7 | 6.8 ± 0.9 | 22.9 ± 0.2 | 26.0 ± 0.9 | 0.0 |
| P19 + DGAT1 | 25.8 ± 1.1 | 0.0 | 10.1 ± 0.4 | 4.4 ± 0.9 | 26.1 ± 1.3 | 26.2 ± 1.4 | 0.2 ± 0.0 |
| P19 + DGAT1 + WRI1 | 22.7 ± 0.9 | 0.0 | 10.1 ± 0.4 | 14.9 ± 0.5 | 27.9 ± 1.3 | 18.5 ± 0.8 | 0.3 ± 0.1 |
| P19 + DGAT1 + FUS3 | 23.9 ± 0.7 | 0.2 ± 0.1 | 7.6 ± 0.4 | 5.3 ± 0.7 | 29.1 ± 0.8 | 26.8 ± 0.7 | 0.4 ± 0.1 |
| P19 + DGAT1 + LEC1 | 24.9 ± 0.4 | 0.1 ± 0.2 | 11.1 ± 0.2 | 4.0 ± 0.1 | 25.9 ± 0.5 | 26.1 ± 0.6 | 0.1 ± 0.0 |
| P19 + DGAT1 + MYB73 | 25.8 ± 0.3 | 0.0 | 10.9 ± 0.7 | 4.3 ± 1.0 | 26.2 ± 0.8 | 25.2 ± 1.8 | 0.1 ± 0.0 |
| P19 | 34.2 ± 4.9 | 0.0 | 10.6 ± 3.1 | 8.3 ± 4.1 | 19.5 ± 1.4 | 23.2 ± 0.8 | 0.1 ± 0.1 |
| P19 + DGAT1 | 27.7 ± 0.1 | 0.3 ± 0.1 | 9.9 ± 1.1 | 4.2 ± 0.3 | 26.4 ± 1.8 | 22.5 ± 0.4 | 0.2 ± 0.1 |
| P19 + DGAT1 + WRI1 | 24.8 ± 1.0 | 0.2 ± 0.0 | 8.8 ± 1.0 | 14.7 ± 0.6 | 27.6 ± 1.0 | 17.2 ± 0.3 | 0.4 ± 0.1 |
| P19 + DGAT1 + bZIP53 | 29.3 ± 0.8 | 0.1 ± 0.2 | 8.7 ± 0.4 | 2.9 ± 0.3 | 22.0 ± 0.5 | 25.9 ± 0.5 | 0.1 ± 0.1 |
| P19 + DGAT1 + AGL15 | 29.2 ± 1.4 | 0.2 ± 0.0 | 4.9 ± 0.9 | 7.0 ± 1.9 | 19.8 ± 0.8 | 30.0 ± 1.3 | 0.3 ± 0.1 |
| P19 + DGAT1 + MYB118 | 31.6 ± 1.7 | 0.2 ± 0.1 | 5.8 ± 1.2 | 4.8 ± 0.8 | 20.7 ± 0.3 | 28.2 ± 1.6 | 0.2 ± 0.1 |
| P19 | 27.4 ± 1.2 | 0.0 | 6.9 ± 1.0 | 4.8 ± 2.6 | 20.0 ± 1.5 | 39.0 ± 4.1 | 0.1 ± 0.0 |
| P19 + DGAT1 | 26.0 ± 1.1 | 0.0 | 8.0 ± 0.6 | 4.2 ± 1.6 | 22.3 ± 2.4 | 33.9 ± 4.3 | 0.2 ± 0.0 |
| P19 + DGAT1 + WRI1 | 23.4 ± 0.8 | 0.1 ± 0.1 | 8.5 ± 0.6 | 17.0 ± 2.4 | 23.3 ± 1.8 | 23.3 ± 4.3 | 0.5 ± 0.1 |
| P19 + DGAT1 + MYB115 | 26.3 ± 0.4 | 0.1 ± 0.1 | 6.6 ± 0.3 | 2.8 ± 0.4 | 22.5 ± 1.8 | 35.7 ± 2.9 | 0.2 ± 0.0 |
| P19 + DGAT1 + TANMEI | 25.6 ± 0.9 | 0.1 ± 0.2 | 8.5 ± 1.2 | 2.6 ± 0.5 | 21.9 ± 2.0 | 35.3 ± 3.8 | 0.2 ± 0.0 |
| P19 + DGAT1 + WUS | 24.3 ± 0.9 | 0.1 ± 0.1 | 5.5 ± 0.6 | 1.7 ± 0.2 | 16.8 ± 1.6 | 47.9 ± 3.3 | 0.2 ± 0.0 |
| P19 | 30.5 ± 1.3 | 0.0 | 8.1 ± 0.9 | 8.2 ± 6.0 | 21.8 ± 1.2 | 28.3 ± 7.3 | 0.1 ± 0.1 |
| P19 + DGAT1 + WRI1 | 25.9 ± 1.7 | 0.2 ± 0.0 | 8.3 ± 0.7 | 19.9 ± 2.8 | 24.5 ± 1.1 | 16.0 ± 0.6 | 0.8 ± 0.1 |
| P19 + DGAT1 + WRI1 + BBM | 27.7 ± 0.7 | 0.2 ± 0.0 | 6.7 ± 0.2 | 21.2 ± 0.7 | 19.8 ± 0.5 | 18.5 ± 0.6 | 0.5 ± 0.1 |
| P19 + DGAT1 + WRI1 + GFR2a1 | 29.2 ± 1.3 | 0.4 ± 0.0 | 6.1 ± 0.1 | 12.9 ± 1.5 | 24.3 ± 0.4 | 20.9 ± 0.5 | 0.4 ± 0.1 |
| P19 + DGAT1 + WRI1 + GFR2a2 | 29.9 ± 2.4 | 0.4 ± 0.1 | 5.5 ± 0.6 | 13.5 ± 2.7 | 23.0 ± 0.5 | 21.3 ± 1.2 | 0.5 ± 0.1 |
| P19 + DGAT1 + WRI1 + PHR1 | 26.2 ± 0.3 | 0.2 ± 0.0 | 4.9 ± 0.0 | 7.6 ± 0.2 | 19.2 ± 0.3 | 36.0 ± 0.7 | 0.3 ± 0.0 |
| P19 | 32.0 ± 1.9 | 1.6 ± 2.7 | 11.1 ± 2.7 | 5.5 ± 2.2 | 23.3 ± 1.1 | 25.4 ± 3.3 | 0.0 |
| P19 + DGAT1 + WRI1 | 27.5 ± 1.2 | 0.7 ± 0.8 | 6.8 ± 0.4 | 16.6 ± 2.1 | 26.7 ± 0.8 | 16.5 ± 0.3 | 1.2 ± 0.2 |
| P19 + DGAT1 + WRI1 + FUS3 | 23.6 ± 1.1 | 2.1 ± 3.5 | 6.5 ± 0.5 | 13.3 ± 0.9 | 32.1 ± 2.6 | 15.6 ± 1.5 | 1.6 ± 0.1 |
| P19 + GFP | 35.8 ± 1.8 | 0.0 ± 0.0 | 8.5 ± 0.8 | 2.0 ± 1.3 | 19.7 ± 1.2 | 32.1 ± 2.2 | 0.03 ± 0.0 |
| P19 + GFP + DGAT1 + WRI1 | 24.6 ± 1.4 | 0.2 ± 0.0 | 10.3 ± 0.5 | 22.7 ± 2.7 | 23.0 ± 1.7 | 14.0 ± 0.6 | 0.99 ± 0.2 |
| P19 + GFP + DGAT1 + NF-YB2 | 27.6 ± 0.6 | 0.1 ± 0.0 | 10.2 ± 0.2 | 3.0 ± 0.2 | 24.1 ± 1.1 | 27.1 ± 1.2 | 0.25 ± 0.0 |
| P19 + GFP + DGAT1 + NF-YB3 | 27.4 ± 0.5 | 0.1 ± 0.0 | 10.8 ± 0.5 | 3.1 ± 1.0 | 24.6 ± 0.9 | 26.0 ± 0.7 | 0.27 ± 0.1 |
| P19 + GFP + DGAT1 + NF-YA3 | 28.9 ± 0.8 | 0.2 ± 0.0 | 8.3 ± 0.4 | 3.6 ± 0.5 | 22.7 ± 1.0 | 29.2 ± 0.9 | 0.17 ± 0.0 |
| P19 + GFP | 38.3 ± 1.3 | 0.0 ± 0.0 | 11.1 ± 1.2 | 2.9 ± 1.4 | 21.3 ± 1.0 | 26.4 ± 3.8 | 0.0 ± 0.0 |
| P19 + GFP + DGAT1 + WRI1 | 29.8 ± 1.1 | 0.3 ± 0.0 | 7.6 ± 1.7 | 18.3 ± 0.6 | 23.9 ± 1.4 | 15.0 ± 0.7 | 1.1 ± 0.5 |
| P19 + GFP + DGAT1 + DOF4 | 32.5 ± 0.5 | 0.0 ± 0.0 | 5.1 ± 0.7 | 3.6 ± 0.2 | 20.5 ± 0.9 | 32.6 ± 1.2 | 0.2 ± 0.1 |
| P19 + GFP + DGAT1 + NF-YB1 | 27.9 ± 0.7 | 0.0 ± 0.0 | 10.8 ± 0.5 | 2.9 ± 0.5 | 27.0 ± 1.3 | 23.7 ± 1.4 | 0.3 ± 0.1 |
| P19 + GFP + DGAT1 + ZFP1 | 25.4 ± 1.4 | 0.1 ± 0.2 | 4.1 ± 0.3 | 5.2 ± 1.2 | 22.8 ± 0.8 | 36.2 ± 0.8 | 0.3 ± 0.1 |
| P19 + GFP | 37.7 ± 1.7 | 0.0 ± 0.0 | 11.5 ± 1.5 | 2.6 ± 2.3 | 22.2 ± 1.9 | 24.1 ± 4.7 | 0.0 ± 0.0 |
| P19 + GFP + DGAT1 + WRI1 | 28.0 ± 2.1 | 0.2 ± 0.0 | 9.3 ± 1.0 | 17.2 ± 3.1 | 27.3 ± 1.2 | 13.0 ± 0.3 | 0.8 ± 0.3 |
| P19 + GFP + DGAT1 + ZF351 | 30.8 ± 0.5 | 0.2 ± 0.1 | 9.5 ± 0.7 | 2.6 ± 1.5 | 25.4 ± 1.1 | 25.4 ± 2.1 | 0.2 ± 0.0 |
| P19 | 18.9 ± 2.9 | 0.4 ± 0.3 | 5.6 ± 1.7 | 6.1 ± 4.8 | 18.3 ± 1.7 | 45.8 ± 9.5 | 0.4 ± 0.1 |
| P19 + DGAT1 + WRI1 | 21.4 ± 2.3 | 0.2 ± 0.0 | 9.9 ± 0.8 | 19.4 ± 0.9 | 20.5 ± 0.9 | 23.8 ± 2.7 | 1.7 ± 0.6 |
| P19 + DGAT1 + WRI1 + ZFP2 | 23.1 ± 1.2 | 0.3 ± 0.1 | 5.3 ± 0.5 | 9.3 ± 1.7 | 16.2 ± 0.7 | 40.5 ± 4.1 | 1.0 ± 0.4 |
| P19 + DGAT1 + WRI1 + ABI5 | 21.4 ± 1.1 | 0.2 ± 0.0 | 8.4 ± 0.7 | 11.4 ± 1.3 | 23.2 ± 1.4 | 29.9 ± 2.9 | 1.2 ± 0.4 |
| P19 + DGAT1 + WRI1 + NF- | 20.5 ± 0.7 | 0.2 ± 0.1 | 9.6 ± 0.4 | 18.1 ± 0.6 | 21.2 ± 0.6 | 25.4 ± 1.5 | 1.6 ± 0.4 |
| YC2 | | | | | | | |

For stable transformation of plants using genes encoding the alternative transcription factors, the following binary constructs are made. The genes for expression of the transcription factors use either the SSU promoter or the SAG12 promoter. Over-expression of embryogenic transcription factors such as LEC1 and LEC2 has been shown to induce a variety of pleotropic effects, undesirable in the present context, including somatic embryogenesis (Feeney et al. (2012); Santos-Mendoza et al. (2005); Stone et al. (2008); Stone et al. (2001); Shen et al. (2010)). To minimize possible negative impact on plant development and biomass yield, tissue or developmental-stage specific promoters are preferred over constitutive promoters to drive the ectopic expression of master regulators of embryogenesis.

### Example 5. Stem-Specific Expression of a Gene Encoding a Transcription Factor

Leaves of N. tabacum plants expressing transgenes encoding WRI1, DGAT and Oleosin contain about 16% TAG at seed setting stage of development. However, the TAG levels were much lower in stems (1%) and roots (1.4%) of the plants (Vanhercke et al., 2014a and b). The inventors considered whether the lower TAG levels in stems and roots were due to poor promoter activity of the Rubisco SSU promoter used to express the gene encoding WRI1 in the transgenic plants. The DGAT transgene in the T-DNA of pJP3502 was expressed by the CaMV35S promoter which is expressed more strongly in stems and roots and therefore was unlikely to be the limiting factor for TAG accumulation in stems and roots.

In an attempt to increase TAG biosynthesis in stem tissue, a construct was designed in which the gene encoding WRI1 was placed under the control of an A. thaliana SDP1 promoter. A 3.156 kb synthetic DNA fragment was synthesized comprising 1.5 kb of the A. thaliana SDP1 promoter (SEQ ID NO:41) (Kelly et al., 2013a and b), followed by the coding region for the A. thaliana WRI1 polypeptide and the (3. max lectin terminator/polyadenylation region. This fragment was inserted between the Sac and NotI sites of pJP3303. The resulting vector was designated pOIL050, which was then used to transform cells from the N. tabacum plants homozygous for the T-DNA from pJP3502 by Agrobacterium-mediated transformation. Transgenic plants were selected for hygromycin resistance and a total of 86 independent transgenic plants were grown to maturity in the glasshouse. Samples were taken from transgenic leaf and stem tissue at seed setting stage and contain increased TAG levels compared to the N. tabacum parental plants transformed with pJP3502.

### Example 6. Effect of Oil Body Protein Expression on Plant Traits

N. tabacum plants transformed with the T-DNA of pJP3502 and expressing transgenes encoding A. thaliana WRI1, DGAT1 and S. indicum Oleosin had increased TAG levels in vegetative tissues. As shown in Example 2 above, when the endogenous gene encoding SDP1 TAG lipase was silenced in those plants, the leaf TAG levels further increased, which indicated to the inventors that substantial TAG turnover was occurring in the plants that retained SDP 1 activity. Therefore, the level of expression of the transgenes in the plants was determined. While Northern hybridisation blotting confirmed strong WRI1 and DGAT1 expression and some oleosin mRNA expression, expression analysis by digital PCR and qRT-PCR detected only very low levels of oleosin transcripts. The expression analysis revealed that the gene encoding the Oleosin was poorly expressed compared to the WRI1 and DGAT1 transgenes. From these experiments, the inventors concluded that the oil bodies in the leaf tissue were not completely protected from TAG breakdown because of inadequate production of Oleosin protein when encoded by the T-DNA in pJP3502. To improve stable accumulation of TAG throughout plant development, several pJP3502 modifications were designed in which the Oleosin gene was substituted. These modified constructs were as follows.
1. pJP3502 contains a gene (SEQ ID NO:42 provides the sequence of its complement) encoding the S. indicum oleosin which was poorly expressed. That gene has an internal UBQ10 intron which might be reducing the expression level. To test this, a 502 bp synthetic DNA fragment containing the S. indicum oleosin gene and lacking the internal UBQ10 intron was synthesized and inserted into pJP3502 as a NotI fragment, to substitute the oleosin gene containing the intron in pJP3502. The resultant plasmid was designated pOIL040.
2. The Rubisco small subunit (SSU) promoter driving expression of the oleosin gene in pJP3502 was replaced by the constitutive enTCUP2 promoter. To this end, a 2321 bp fragment containing the enTCUP2 promoter, Oleosin protein coding region, G. max lectin terminator/polyadenylation region and the first 643 bp of the downstream SSU promoter driving wril expression was synthesized and subcloned into the AscI and SpeI sites of pJP3502 resulting in pOIL038.
3. A similar strategy was followed for the expression of an engineered version of the S. indicum oleosin gene containing 6 introduced cysteine residues (03-3) under the control of the enTCUP2 promoter (Winichayakul et al., 2013). A 2298 bp fragment containing the enTCUP2 promoter, Oleosin 03-3 protein coding region, G. max lectin terminator/polyadenylation region and the first 643 bp of the downstream SSU promoter driving wril expression was synthesized and subcloned into the AscI and SpeI sites of pJP3502 resulting in pOIL037.
4. The NotI sites flanking the S. indicum oleosin gene in pJP3502 were used to exchange the protein coding region for one encoding peanut Oleosin3 (Accession No. AAU21501.1) (Parthibane et al., 2012a and b). A 528 bp fragment containing the oleosin3 gene, flanked by NotI sites, was synthesized and subcloned into the respective site of pJP3502. The resulting vector was designated pOIL041.
5. Similarly, a 1077 bp NotI flanked fragment containing the gene coding for the A. thaliana steroleosin (Arab-1) (Accession No. AAM10215.1) (Jolivet et al., 2014) was synthesized and subcloned into the NotI site of pJP3502, resulting in pOIL043.
6. The Nannochloropsis oceanic lipid droplet surface protein (LDSP) (Accession No. AFB75402.1) (Vieler et al., 2012) was synthesized as a 504 bp NotI-flanked fragment and subcloned into the NotI site of pJP3502, yielding pOIL044.
7. Finally, the A. thaliana caleosin (CLO3) (Accession No. 022788.1) (Shimada et al., 2014) was synthesized as a 612 bp NotI flanked fragment and subcloned into pJP3502, resulting in pOIL042.

Each of these constructs was introduced into N. benthamiana leaf cells as described in Example 1. Transient expression of both pJP3502 and pOIL040 in N. benthamiana leaf tissue resulted in elevated TAG levels and similar changes in the TAG fatty acid profile but pOIL040 increased the TAG level more (1.3% compared to 0.9%). Each of the constructs pOIL037, pOIL038, pOIL041, pOIL042 and pOIL043 were used to stably transform N. tabacum plants (cultivar W38) by Agrobacterium-mediated methods. Transgenic plants were selected on the basis of kanamycin resistance and are grown to maturity in the glasshouse. Samples are taken from transgenic leaf tissue at different stages during plant development and contain increased TAG levels compared to wild-type N. tabacum and N. tabacum plants transformed with pJP3502.

Cloning and Characterisation of LDAP Polypeptides from Sapium sebifera

Oleosins are not highly expressed in non-seed oil accumulating plant tissues such as the mesocarp of olive, oil palm, and avocado (Murphy, 2012). Instead, lipid droplet associated proteins (LDAP) have been identified in these tissues that may play a similar role to that of oleosin in seed tissues (Horn et al., 2013). The inventors therefore considered it possible that oleosin might not be the optimal packaging protein to protect the accumulated oil from TAG lipase or other cytosolic enzyme activities in vegetative tissues of plants. LDAP polypeptides were therefore identified and evaluated for enhancement of TAG accumulation, as follows.

The fruit of Chinese tallow tree, Sapium sebifera, a member of the family Euphorbiaceae, was of particular interest to the inventors as it contains an oil-rich tissue outside of the seed. A recent study (Divi et al, submitted for publication) indicated that this oleoginous tissue, called a tallow layer, might be derived from the mesocarp of its fruit. Therefore, the inventors queried the transcriptome of S. sebifera for LDAP sequences. A comparative analysis of expressed genes in the fruit coat and seed tissues revealed a group of three previously unidentified LDAP genes which were highly expressed in the tallow layer.

Nucleotide sequences encoding the three LDAPs were obtained by RT-PCR using RNAs derived from tallow tissue using three pairs of primers. The primer sequences were based on the DNA sequences flanking the entire coding region of each of the three genes. The primer sequences were: for LDAP1,5′-TTTTAACGATATCCGCTAAAGG-3′ (SEQ ID NO:76) and 5′-AATGAATGAACAAGAATTAAGTC-3′ (SEQ ID NO:77) AT-3′; LDAP2,5′-CTTTTCTCACACCGTATCTCCG-3′ (SEQ ID NO:78) and 5′-AGCATGATATA CTTGTCGAGAAAGC-3′ (SEQ ID NO:79); LDAP3,5′-GCGACAGTGTAGCGTTTT-3′ (SEQ ID NO:80) and 5′-ATACATAAAATGAAAACTATTGTGC-3′ (SEQ ID NO:81).

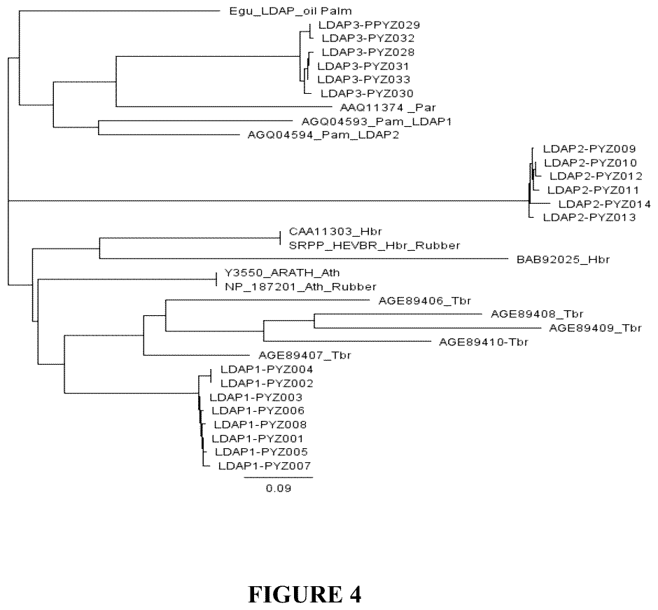

Analysis of the S. sebifera transcriptome revealed multiple orthologs for each of the LDAP genes, including eight LDAP1, six LDAP2, and six LDAP3 genes, with less than 10% sequence divergence within each gene family. The putative peptide sequences were aligned and a phylogenetic tree was constructed using Genious software (FIG. 4), together with LDAPs homologs from other plant species, including two from avocado (Pam), one from oil palm, one from Parthenium argentatum (Par), two from Arabidopsis(Ath), five from Taraxacum brevicorniculatum (Tbr), three from Hevea brasiliensis (Hbr), as presented in FIG. 4. The phylogenetic tree was revealed that the SsLDAP3 shared greater amino acid sequence identity to the LDAP1 and LDAP2 polypeptides from avocado and the LDAP from oil palm, while the SsLDAP1 and SsLDAP2 polypeptides were more divergent.

### Genetic Constructs for Over-Expression of LDAP

In order to test the function of the LDAPs from S. sebifera, expression vectors were made to express each of these polypeptides under the control of the 35S promoter in leaf cells. The full length SsLDAP cDNA sequences were inserted into the pDONR207 destination vector by recombination reactions, replacing the CcdB and Cm(R) regions of the destination vector with the SsLDAP cDNA fragments. Following confirmation by restriction digestion analysis and DNA sequencing, the constructs were introduced into Agrobacterium tumefaciens strain AGL1 and used for both transient expression in N. benthamiana leaf cells and stable transformation of N. tabacum.

The expression of each of the three SsLDAP genes under the transcriptional control of the 35S promoter in N. benthamiana leaves in combination with the expression of 35S::AtDGAT1 and 35S::AtWRI1 yielded substantially higher levels of TAG accumulation relative to the cells infiltrated with the 35S::AtDGAT1 and 35S::AtWRI1 genes without the LDAP construct. The TAG level was increased about 2-fold above the TAG level in the control cells. A significant increase in the level of α-linolenic acid (ALA) and a reduced level of saturated fatty acids was observed in the cells receiving the combination of genes, relative to the control cells.

Co-Localisation of YFP-Fused LDAP Polypeptides with Lipid Droplets in Leaf Cells

In order to characterise SsLDAPs in vivo and observe their dynamic behaviour, expression constructs were made for expression of fusion polypeptides consisting of the LDAP polypeptides fused to yellow fluorescent protein (YFP). For each fusion polypeptide, the YFP was fused in-frame to the C-terminus of the SsLDAP. The full open reading frame of each of the three LDAP genes without a stop codon, at its 3′ end, was fused to the YFP sequence and the chimeric genes inserted into pDONR207. Following confirmation of the resultant constructs by restriction digestion and DNA sequencing, the constructs were introduced into A. tumefaciens strain AGL1 and used for both transient expression in N. benthamiana leaf cells and stable transformation of N. tabacum. Three days following infiltration of the leaf cells with the LDAP-YFP constructs, leaf discs from the infiltrated zones were stained with Nile Red, which positively stained lipid droplets, and observed under a confocal microscope to detect both the red stain (lipid droplets) and fluorescence from the YFP polypeptide. Co-localisation of LDAP-YFP with the lipid droplets was observed, indicating that the LDAP associated with the lipid droplets in the leaf cells.

### Example 7. Modifying Traits in Monocotyledonous Plants—Expression in Leaves and Stems

A series of binary expression vectors was designed for Agrobacterium-mediated transformation of sorghum (S. bicolor) and wheat (Triticum aestivum) to increase the oil content in vegetative tissues. The starting vectors for the constructions were pOIL093-095, pOIL134 and pOIL 100-104 (see Example 5 of WO 2016/004473). Firstly, a DNA fragment encoding the Z. mays WRI1 polypeptide was amplified by PCR using pOIL 104 as a template and primers containing KpnI restriction sites. This fragment was subcloned downstream of the constitutive Oryza sativa Actin1 promoter of pOIL095, using the KpnI site. The resulting vector was designated pOIL 154. The DNA fragment encoding the Umbelopsis ramanniana DGAT2a under the control of the Z. mays ubiquitin promoter (pZmUbi) was isolated from pOIL 134 as a NotI fragment and inserted into the NotI site of pOIL 154, resulting in pOIL155. An expression cassette consisting of the PAT coding region under the control of the pZmUbi promoter and flanked at the 3′ end by the A. tumefaciens NOS terminator/polyadenylation region was constructed by amplifying the PAT coding region using pJP3416 as a template. Primers were designed to incorporate BamHI and SacI restriction sites at the 5′ and 3′ ends, respectively. After BamHI+SacI double digestion, the PAT fragment was cloned into the respective sites of pZLUbi1casNK. The resulting intermediate was designated pOIL 141. Next, the PAT selectable marker cassette was introduced into the pOIL 155 backbone. To this end, pOIL141 was first cut with NotI, blunted with Klenow fragment of DNA polymerase I and subsequently digested with AscI. This 2622 bp fragment was then subcloned into the ZraI-AscI sites of pOIL 155, resulting in pOIL 156. Finally, the Actin1 promoter driving WRI1 expression in pOIL 156 was exchanged for the Z. mays Rubisco small subunit promoter (pZmSSU) resulting in pOIL 157. This vector was obtained by PCR amplification of the Z. mays SSU promoter using pOIL 104 as a template and flanking primers containing AsiSI and PmlI restriction sites. The resulting amplicon was then cut with SpeI+MluI and subcloned into the respective sites of pOIL 156.

These vectors therefore contained the following expression cassettes:

pOIL 156: promoter O. sativa Actin1::Z. mays WRI1, promoter Z. mays Ubiquitin::U. rammaniana DGAT2a and promoter Z. mays Ubiquitin: PAT

pOIL 157: promoter Z. mays SSU::Z. mays WRI1, promoter Z. mays Ubiquitin::U. rammaniana DGAT2a and Z. mays Ubiquitin:: PAT.

A second series of binary expression vectors containing the Z. mays SEE1 senescence promoter (Robson et al., 2004, see Example 5 of WO 2016/004473), Z. mays LEC1 transcription factor (Shen et al., 2010) and a S. bicolor SDP1 hpRNAi fragment were constructed as follows. First, a matrix attachment region (MAR) was introduced into pORE04 by AatII+SnaBI digest of pDCOT and subcloning into the AatII+EcoRV sites of pORE04. The resulting intermediate vector was designated pOIL158. Next, the PAT selectable marker gene under the control of the Z. mays Ubiquitin promoter was subcloned into pOIL 158. To this end, pOIL141 was first digested with NotI, treated with Klenow fragment of DNA polymerase I and finally digested with AscI. The resulting fragment was inserted into the AscI+ZraI sites of pOIL158, resulting in pOIL159. The original RK2 oriV origin of replication in pOIL159 was exchanged for the RiA4 origin by SwaI+SpeI restriction digestion of pJP3416, followed by subcloning into the SwaI+AvrII sites of pOIL 159. The resulting vector was designated pOIL160. A 10.019 kb ‘Monocot senescence part1’ fragment containing the following expression cassettes was synthesized: O. sativa Actin1::A. thaliana DGAT1, codon optimized for Z. mays expression, Z. mays SEE1::Z. mays WRI1, Z. mays SEE1: Z. mays LEC1. This fragment was subcloned as a SpeI-EcoRV fragment into the SpeI-StuI sites of pOIL 160, resulting in pOIL 161. A second 7.967 kb ‘Monocot senescence part2’ fragment was synthesized and contains the following elements: MAR, Z. mays Ubiquitin:: hpRNAi fragment targeted against S. bicolor/T. aestivum SDP1, empty cassette under the control of the O. sativa Actin1 promoter. The sequences of two S. bicolor SDP1 TAG lipases (Accession Nos. XM 002463620; SEQ ID NO:73 and XM_002458486; SEQ ID NO:38) and one T. aestivum SDP1 sequence (Accession No. AK334547) (SEQ ID NO:74) were obtained by a BLAST search with the A. thaliana SDP1 sequence (Accession No. NM_120486). A synthetic hairpin construct (SEQ ID NO:75) was designed including four fragments (67 bp, 90 bp, 50 bp, 59 bp) of the S. bicolor XM_002458486 sequence that showed highest degree of identity with the T. aestivum SDP1 sequence. In addition, a 278 bp fragment originating from the S. bicolor XM_002463620 SDP1 lipase was included to increase silencing efficiency against both S. bicolor SDP1 sequences. The ‘Monocot senescence part2’ fragment is subcloned as a BsiWI-EcoRV fragment into the BsiWI-FspI sites of pOIL 161. The resulting vector is designated pOIL 162.

The genetic constructs pOIL 156 pOIL 157, pOIL 161 and pOIL 162 are used to transform S. bicolor and T. aestivum using Agrobacterium-mediated transformation. Transgenic plants are selected for hygromycin resistance and contain elevated levels of TAG and TFA in vegetative tissues compared to untransformed control plants. Such plants are useful for providing feed for animals as hay or silage, as well as producing grain, or may be used to extract oil.

Further genetic constructs are made for expression of combinations of polypeptides in leaves and stems of monocotyledonous plants, including the C4-photosynthesis plants S. bicolor and Z. mays. Several constructs are made containing genes for expression of WRI1, DGAT and oleosin, with each gene under the control of a constitutive promoter such as a maize Ubiquitin gene promoter or a rice actin gene promoter, and containing an NPTII gene as selectable marker gene. In one particular construct, the WRI1 is sorghum WRI1. In another, the oleosin is SiOleosinL (see Example 9). In other particular constructs, the oleosin gene is replaced with a gene encoding either LDAP2 or LDAP3 from S. sebifera (Example 6). These constructs are used as the “core constructs” for transformation of S. bicolor and Z. mays and are deployed on their own or in combination with genetic constructs for expression of a hairpin RNA targeting one or more SDP1 genes in sorghum or maize (see above), a construct encoding Lec2 under the control of a SEE1 promoter (senescence specific), or both. Another construct is made comprising three genes, namely for expression of a hairpin RNA targeting the endogenous TGD5 gene to reduce its expression, a FatA fatty acyl thioesterase and a PDAT, which is used to increase the level of TAG and/or the TTQ parameter for plants transformed with this construct.

### Example 8. Extraction of Oil

Extraction of Lipid from Leaves

Transgenic tobacco leaves which had been transformed with the T-DNA from pJP3502 were harvested from plants grown in a glasshouse during the summer months. The leaves were dried and then ground to 1-3 mm sized pieces prior to extraction. The ground material was subject to soxhlet (refluxing) extraction over 24 hours with selected solvents, as described below. 5 g of dried tobacco leaf material and 250 ml of solvent was used in each extraction experiment.

### Hexane Solvent Extraction

Hexane is commonly used as a solvent commercially for oil extraction from pressed oil seeds such as canola, extracting neutral (non-polar) lipids, and was therefore tried first. The extracted lipid mass was 1.47 g from 5 g of leaf material, a lipid recovery of 29% by weight. 1H NMR analysis of the hexane extracted lipid in DMSO was preformed. The analysis showed typical signals for long chain triglyceride fatty acids, with no aromatic products being present. The lipid was then subjected to GCMS for identification of major components. Direct GCMS analysis of the hexane extracted lipid proved to be difficult as the boiling point was too high and the material decomposed in the GCMS. In such situations, a common analysis technique is to first make methyl esters of the fatty acids, which was done as follows: 18 mg lipid extract was dissolved in 1 mL toluene, 3 mL of dry 3N methanolic HCL was added and stirred overnight at 60° C. 5 mL of 5% NaCl and 5 mL of hexane were added to the cooled vial and shaken. The organic layer was removed and the extraction was repeated with another 5 mL of hexane. The combined organic fractions were neutralized with 8 mL of 2% KHCO3, separated and dried with Na2SO4. Solvent was evaporated under a stream of N2 and then made up to a concentration of 1 mg/mL in hexane for GCMS analysis. Main fatty acids present were 16:0 (palmitic, 38.9%) and 18:1 (oleic, 31.3%) (Table 10).

TABLE 10
Fatty acid composition in transgenic tobacco leaves

| | FA | | | | | | |
|---|---|---|---|---|---|---|---|
| | 16:0 | 16:1 | 18:0 | 18:1 | 18:2 | 20:0 | 22:0 |
| % wt | 38.9 | 4.6 | 6.4 | 31.3 | 2.5 | 1.5 | 0.6 |

### Acetone Solvent Extraction

Acetone was used as an extraction solvent because its solvent properties should extract almost all lipid from the leaves, i.e. both non-polar and polar lipids. The acetone extracted oil looked similar to the hexane extracted lipid. The extracted lipid mass was 1.59 g from 5 g of tobacco leaf, i.e. 31.8% by weight. 1H NMR analysis of the lipid in DMSO was performed. Signals typical of long chain triglyceride fatty acids were observed, with no signal for aromatic products.

### Hot Water Solvent Extraction

Hot water was attempted as an extraction solvent to see if it was suitable to obtain oil from the tobacco leaves. The water extracted material was gel like in appearance and gelled when cooled. The extracted mass was 1.9 g, or 38% by weight. This material was like a thick gel and was likely to have included polar compounds from the leaves such as sugars and other carbohydrates. The 1H NMR analysis of the material in DMSO was preformed. The analysis showed typical signals for long chain triglyceride fatty acids, with no aromatic products being extracted. The left over solid material was extracted with hexane, yielding 20% of lipid by weight, indicating that the water extraction had not efficiently extracted non-polar lipids.

### Ethanol Solvent Extraction

Ethanol was used as an extraction solvent to see if it was suitable to obtain oil from the tobacco leaves. The ethanol extracted lipid was similar in appearance to both the water- and hexane-extracted lipid, being yellow-red in colour, had a gel-like appearance and gelled when cooled. The extracted lipid mass was 1.88 g from 5 g tobacco, or 37.6% by weight. The ethanol solvent would also have extracted some of the polar compounds in the tobacco leaves.

### Ether Solvent Extraction

Diethyl ether was attempted as an extraction solvent since it was thought that it might extract less impurities than other solvents. The extraction yielded 1.4 g, or 28% by weight. The ether extracted lipid was similar to the hexane extracted material in appearance, was yellowish in colour, and it did appeared a little cleaner than the hexane extract. While the diethyl ether extraction appeared to have given the cleanest oil, the NMR analysis showed a mixture of more organic compounds.

### Example 9. Expression of Oil Body Proteins in Plant Vegetative Tissues

A protein coding region encoding a Rhodococcus opacus TadA lipid droplet associated protein (MacEachran et al. 2010; Accession number HM625859), codon optimized for expression in dicotyledonous plants such as Nicotiana benthamiana, was synthesized as a NotI-SpeI DNA fragment. The fragment was inserted downstream of the 35S promoter in pJP3343 using the NotI-SpeI sites. The resultant plasmid was designated pOIL380. A protein coding region encoding a Sesame indicum OleosinL lipid droplet associated protein (Tai et al. 2002; Accession number AF091840; SEQ ID NO:86) was synthesized as a NotI-SacI DNA fragment and inserted downstream of the 35S promoter in pJP3343 using the same sites. The resultant plasmid was designated pOIL382. A protein coding region encoding a Sesame indicum OleosinH1 lipid droplet associated protein (Tai et al., 2002; Accession number AF302807) was synthesized as a NotI-SacI DNA fragment and cloned downstream of the 35S promoter in pJP3343 using the same sites. The resultant plasmid was designated pOIL383. A variant of the protein coding region encoding S. indicum OleosinH1 having three amino acid substitutions to remove ubiquitination sites (K130R, K143R, K145R) (Hsiao and Tzen, 2011) was generated by targeted mutagenesis. The coding region was inserted downstream of the 35S promoter in pJP3343 as a NotI-SacI fragment. The resultant plasmid was designated pOIL384. A protein coding region encoding a Vanilla planifolia leaf OleosinU1 lipid droplet associated protein (Huang and Huang, 2016; Accession number SRX648194) was codon optimized for expression in N. benthamiana, synthesized as a SpeI-EcoRI DNA fragment and inserted downstream of the 35S promoter in pJP3343 using the same sites. The resultant plasmid was designated pOIL386. A protein coding region encoding a Persea americana mesocarp OleosinM lipid droplet associated protein (Huang and Huang 2016; Accession number SRX627420) was codon optimized for expression in N. benthamiana, synthesized as a SpeI-EcoRI DNA fragment and inserted downstream of the 35S promoter in pJP3343 using the same restriction sites. The resultant plasmid was designated pOIL387. A protein coding region encoding an Arachis hypogaea Oleosin 3 lipid droplet associated protein (Parthibane et al., 2012a; Accession number AY722696) was codon optimized for expression in N. benthamiana, flanked by NotI sites and inserted into the binary expression vector pJP3502. The resulting plasmid, pOIL041, was digested with NotI and the resultant 520 bp DNA fragment was inserted downstream of the 35S promoter of pJP3343. The resultant plasmid was designated pOIL 190. Similarly, the protein coding region for the A. thaliana Caleosin3 lipid droplet associated protein (Shen et al., 2014; Laibach et al., 2015; Accession number AK317039) was codon optimized for expression in N. benthamiana, flanked by NotI sites and inserted into pJP3502. The resulting plasmid, pOIL042, was digested with NotI and the resulting 604 bp DNA fragment was inserted downstream of the 35S promoter of pJP3343. The resultant plasmid was designated pOIL 191. A protein coding region encoding an A. thaliana steroleosin lipid droplet associated protein (Accession number AT081653) was codon optimized for expression in N. benthamiana, flanked by NotI sites and inserted into pJP3502. The resultant plasmid, pOIL043, was digested with NotI and the resultant 1069 bp DNA fragment was inserted downstream of the 35S promoter of pJP3343. The resultant plasmid was designated pOIL 192. A protein coding region encoding a Nannochloropsis oceanica LSDP oil body protein (Vieler et al., 2012; Accession number JQ268559) was codon optimized for expression in N. benthamiana, flanked by NotI sites and inserted into the pJP3502 binary expression vector. The resultant plasmid, pOIL044, was digested with NotI and the 496 bp DNA fragment was inserted downstream of the 35S promoter of pJP3343. The resultant plasmid was designated pOIL 193. A protein coding region encoding a Trichoderma reesei HFBI hydrophobin (Linder et al., 2005; Accession number Z68124) was codon optimized for expression in N. benthamiana, flanked by NotI sites and inserted into pJP3502. The resultant plasmid, pOIL045, was digested with NotI and the 313 bp DNA fragment was inserted downstream of the 35S promoter of pJP3343. The resultant plasmid was designated pOIL194. An ER-targeted variant of the Trichoderma reesei HFBI hydrophobin was created by amending the KDEL ER retention peptide to the C-terminus (Gutierrew et al., 2013). This variant was codon optimized for expression in N. benthamiana and cloned as a NotI fragment into pJP3502, resulting in pOIL046. Subsequently, pOIL046 was digested with NotI and the 325 bp fragment was inserted into pJP3343. The resulting vector was designated pOIL 195.

Each of the genetic constructs encoding the lipid droplet associated polypeptides were introduced into N. benthamiana leaves in combination with genetic constructs encoding WRI1, DGAT1 and p19 as described in Example 1 with some minor modifications. Agrobacterium tumefaciens cultures containing the gene coding for the p19 silencing suppressor protein and the chimeric genes of interest were mixed such that the final OD600 of each culture was equal to 0.125 prior to infiltration. Samples being compared were located on the same leaf. After infiltration, N. benthamiana plants were grown for a further five days before leaf discs were harvested, pooled across three leaves from the same plant, freeze-dried, weighed and stored at −80° C.. Total lipids were extracted from freeze-dried tissues using chloroform:methanol:0.1 M KCl (2:1:1 v/v/v) and aliquots loaded on a thin layer chromatography (TLC) plate and developed in hexane:diethyl ether:acetic acid (70:30:1, v/v/v). TAG was recovered, converted to FAME in the presence of a known amount of triheptadecanoin (Nu-Chek PREP, Inc. USA) as internal standard for lipid quantitation, and analyzed by GC-FID.

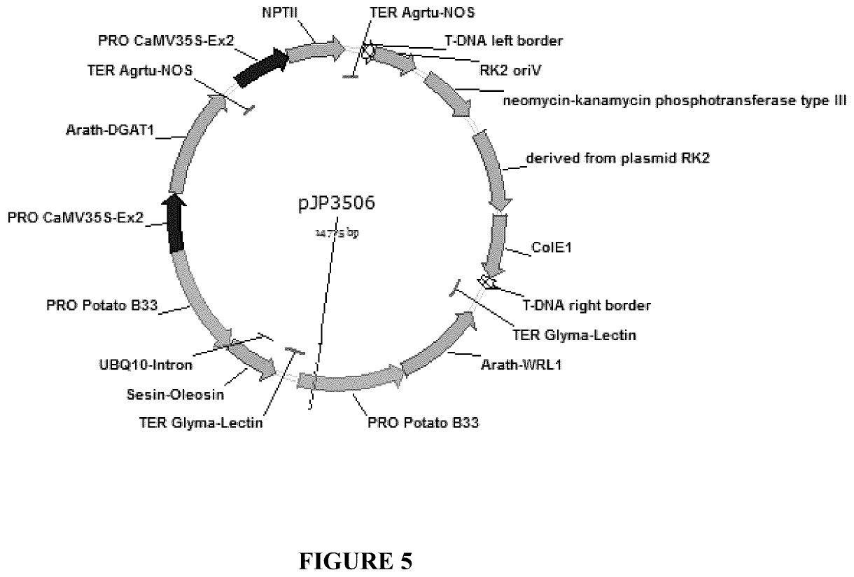

The assays showed a range of TAG levels compared to the WRI1+DGAT1 control. Some constructs encoding lipid droplet associated polypeptides increased the TAG level relative to the control in some assays whereas others did not. A consistent and statistically significant increase in TAG content was observed when the construct expressing SiOleosinL (pOIL382) was introduced (FIG. 5); this construct was superior to all the others tested in these assays. In one experiment, the increase was 2.27-fold compared to p19+WRI+DGAT and 121.7-fold compared to the p19 control. An increase in the levels of C18:2 and C18:1 (about 22% increased) and a decrease in C16:0 (about 23% decreased) was also observed in the TAG for this construct, relative to the p19+WRI1+DGAT1 control (FIG. 5). Microscopic analyses to visualize lipid droplets in the leaf cells expressing SiOleosinL showed a decrease in lipid droplet size and an increase in abundance compared to the control.

The lipid droplets in leaf cells transiently expressing the genes encoding SiOleosinL together with p19+WRI1+DGAT1 were examined by microscopy. N. benthamiana treated leaf discs were collected 4 days after infiltration. Each leaf sample was prepared, stained and imaged within 30-45 minutes, to ensure the samples were imaged fresh. More specifically, immediately after collection, the abaxial epidermis was peeled off in 50 mM PIPES pH7. One half of each disc was stained for 10 minutes in 2 μg/ml BODIPY505/515 in 50 mM PIPES pH7, followed by 2-3 washes in 50 mM PIPES pH7. During this time, the other disc half was kept in 50 mM PIPES pH7. Leaf tissue was mounted in 50 mM PIPES pH7 and imaged immediately, using a Leica SP8 Laser-Scanning Confocal Microscope, a 20× objective (NA=0.75), and the LAS X software. Lipid droplets and chloroplasts were imaged by exciting the leaf discs with a 505 nm laser. BODIPY 505/515 signal was collected between 510 and 540 nm, while chloroplast signal was collected between 650 nm and 690 nm. Unstained half discs were imaged to determine tissue auto-fluorescence.

Microscopy of cells in the leaf discs having the introduced SiOleosinL showed an accumulation of smaller lipid droplets compared to the discs having the p19+WRI1+DGAT1 without SiOleosinL. In contrast, leaf cells expressing genes encoding the p19+WRI1+DGAT1+SiOleosinH combination showed larger lipid droplets which looked about the same as those observed in leaves expressing p19+WRI1+DGAT1 without an oleosin. Finally, when genes encoding both SiOleosinH and SiOleosinL were co-expressed with p19+WRI1+DGAT1, the lipid droplets were smaller and looked similar to those observed in leaves expressing p19+WRI1+DGAT1+SiOleosinL. Interestingly, expression of the vanilla leaf oleosin (pOIL386) resulted in a different pattern in which lipid droplets appeared compacted in a smear form.

Further assays were carried out using radiolabelled [¹⁴C]-acetate to measure the rate of TAG synthesis for the different gene combinations including each of the lipid droplet associated polypeptides. The [¹⁴C]-acetate was infiltrated into the same leaf tissues at 3 days post-infiltration of the genetic constructs i.e. after the genes had been expressed for three days. Leaf discs were sampled after 5 min, 10 min and 3 hr after addition of the radiolabel, and total lipids in the tissues were extracted and fractionated by TLC. The amount of radioactivity in different lipid types was quantitated using a Fujifilm FLA-5000 phosphorimager or using a Beckman-Coulter LS 6500 Multipurpose Scintillation Counter.

These assays demonstrated an increase in TAG synthesis rates in the leaves expressing SiOleosinL (pOIL382) as well as an increase in PC and PA synthesis rates over the three hours in leaves expressing SiOleosinL. SiOleosinL expression increased TAG accumulation already at 15 minutes (789 dpm) compared to p19 (198 dpm). In N. benthamiana leaf cells expressing genes encoding the p19+WRI1+DGAT1 combination, TAG accumulated rapidly, reaching 3865 dpm after 5 min of [¹⁴C]-acetate incorporation compared to 293 dpm in the p19 control. This accumulation reached a maximum at 10 minutes after [¹⁴C]-acetate addition (4519 dpm). However, the radiolabel in TAG quickly reduced thereafter to reach 1013 dpm at 15 minutes, indicating TAG catabolism when the gene encoding SiOleosinL was added, the TAG was stabilised, indicating protection (i.e. TAG packaging) in the leaf cells. TAG rapidly accumulated at 5 minutes of infiltration (2855 dpm) and the level remained the same at 10 and 15 min after [¹⁴C]-acetate addition. At the 15 min timepoint, TAG accumulation was equivalent to 2690 dpm for the p19+WRI1+DGAT1+SiOleosinL combination compared to 1013 dpm for the p19+WRI1+DGAT1 combination.

TAG degradation was not correlated with free fatty acid (FFA) levels, presumably because of further catabolism or of incorporation into lipids other that TAG. In order to study TAG degradation and chase the resulting derivatives, [¹⁴C]-acetate incorporation into TAG and its stability at 3 hr post-addition was studied. This experiment showed an increase in [¹⁴C] in PC (2579 dpm) and PA (1270 dpm) in leaf cells expressing the SiOleosinL construct compared to 1495 dpm PC and 899 PA in both p19 and p19+WRI1+DGAT1 controls.

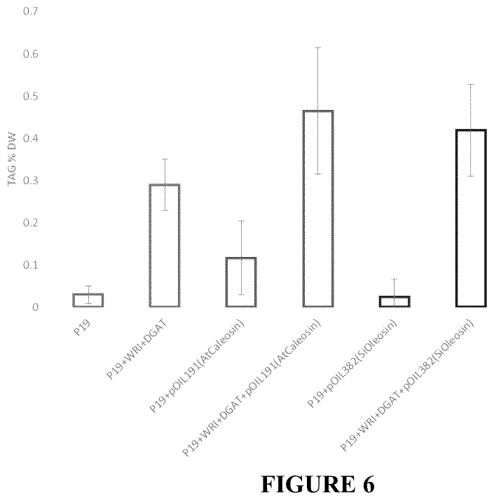

In another experiment, pOIL191 (AtCaleosin 3) was transiently expressed in N. benthamiana leaves. The expression of this gene increased TAG content by 3.6 fold (FIG. 6) compared to p19 control. The expression of At Caleosin3 with WRI1 and DGAT1 resulted in a further increase in TAG content by up to 15.3 fold compared p19 control, and up to 1.6 fold compared to WRI1 and DGAT1 control. TAG yields are comparable with SiOleosin co-expression with WRI1 and DGAT1.

### Example 10. Medium-Chain Fatty Acid Production in Vegetative Plant Cells

Eccleston et al. (1996) studied the accumulation of C12:0 and C14:0 fatty acids in both seeds and leaves of transgenic Brassica napus plants transformed with a constitutively expressed gene encoding California Bay Laurel 12:0-ACP thioesterase (Umbellularia californica). That study reported that substantial levels of C12:0 accumulated in mature B. napus seeds but only very low levels of C12:0 were observed in leaf tissue, despite high levels of 12:0-ACP thioesterase expression and activity. The same results were obtained when the gene was transformed into A. thaliana (Voelker et al., 1992). That research was extended by the co-expression of the Cocos nucifera LPAAT and Umbellularia californica thioesterase which resulted in an increased accumulation of total C12:0 as well as an increased fraction of trilaurin in the seeds of B. napus (Knutzon et al., 1999). The prior art therefore indicated that medium chain fatty acids (MCFA) synthesis in vegetative plant cells was problematic.

To test the effect of introducing thioesterases having specificity for MCFA in combination with other genes described herein, chimeric DNAs for expressing several different thioesterases were synthesized and introduced into plant cells either singly or in combinations. The protein coding regions for thioesterases from organisms known to produce MCFAs (Jing et al., 2011) were synthesised and inserted as EcoRI fragments into the binary vector pJP3343 which contained a 35S-promoter expression cassette (Vanhercke et al., 2013). The thioesterases were: Cinnamomum camphora 14:0-ACP thioesterase (referred to as Cinca-TE) (Yuan et al., 1995; Accession No. Q39473.1; SEQ ID NO:43), Cocos nucifera acyl-ACP thioesterase FatB1 (Cocnu-TE1; Accession No. AEM72519.1; SEQ ID NO:88), Cocos nucifera acyl-ACP thioesterase FatB2 (Cocnu-TE2; Accession No. AEM72520.1; SEQ ID NO:89), Cocos nucifera acyl-ACP thioesterase FatB3 (Cocnu-TE3; Accession No. AEM72521.1; SEQ ID NO:90), Cuphea lanceolata acyl-(ACP) thioesterase type B (Cupla-TE) (Topfer et al., 1995; Accession No. CAB60830.1; SEQ ID NO: 91), Cuphea viscosissima FatB1 (Cupvi-TE; Accession No. AEM72522.1; SEQ ID NO:92) and Umbellularia californica 12:0-ACP thioesterase (Umbca-TE) (Voelker et al., 1992; Accession No. Q41635.1; SEQ ID NO:93). These thioesterases were all in the FATB class and had specificity for MCFA. The protein coding regions for C. nucifera LPAAT (Cocnu-LPAAT, MCFA type) (Knutzon et al., 1995; Accession No. Q42670.1; SEQ ID NO:94) and A. thaliana plastidial LPAAT1 (Arath-PLPAAT; Accession No. AEE85783.1; SEQ ID NO:95), were also cloned. Cocnu-LPAAT had previously been shown to increase MCFA incorporation on the sn-2 position of TAG in seeds (Knutzon et al., 1995) whilst A. thaliana plastidial LPAAT (Arath-PLPAAT) (Kim et al., 2014) was used as a control LPAAT to determine the effect of any MCFA specificity that the Cocnu-LPAAT might have. The former LPAAT uses acyl-CoA as one substrate and operates in the ER in its native context, whereas the latter PLPAAT uses acyl-ACP as substrate and works in the plastid.

The thioesterase genes were introduced into Nicotiana benthamiana leaves by Agrobacterium-mediated infiltration as described in Example 1 along with the gene for co-expression of the p19 silencing suppressor and either the Cocnu-LPAAT or Arath-PLPAAT to determine whether MCFA could be produced in N. benthamiana leaf tissue. Infiltrated leaf zones were harvested and freeze-dried five days after infiltration with the Agrobacterium mixtures, after which the total fatty acid content and composition were determined by GC as described in Example 1 (Table 11). For the data shown in Table 11, errors are the standard deviation of triplicate infiltrations. The infiltrated zones of control leaves contained only trace (<0.1%) or zero levels of fatty acids C12:0 and C14:0 whereas C16:0 was present at 14.9%±0.6 of the TFA in the total leaf lipids. C12:0 levels were only increased significantly by expression of the Cocnu-TE3 (1.2%±0.1) and Umbca-TE (1.6%±0.1). Expression of each of the tested thioesterases resulted in the accumulation of C14:0 in the N. benthamiana leaves, with Cinca-TE giving the highest level of 11.3%±1.0. Similarly, expression of each of the thioesterases with the exception of Umbca-TE resulted in increased C16:0 levels. The highest level of C16:0 accumulation (35.4%±4.7) was observed with expression of Cocnu-TE1. Substantial necrosis of the infiltrated zones was observed in the leaves when the FATB genes were expressed alone, which appeared to correlate with the level of MCFA production. The inventors considered that the necrosis was probably due to levels of free fatty acids (FFA) greater than optimum, and also due to the extensive accumulation of MCFA in phospholipid lipid pools rather than in TAG.

TABLE 11
Total leaf fatty acid composition (% total leaf fatty acid) of selected fatty acids in
Nicotiana benthamiana leaves infiltrated with various thioesterases (TE) and LPAATs.
Results are grouped by the co-infiltrated gene (single genes (other than p19 present
in all samples), Arath-LPAAT + various TE, Cocnu-LPAAT + various TE). ‘Control’
denotes uninfiltrated N. benthamiana leaf whereas ‘p19 only’ contains the
silencing suppressor gene alone. 16:3 is 16:3^{Δ7,10,13}; 18:3 is 18:3^{Δ9,12,15}.
Gene identities are defined in the text.

| | | 12:0 | 14:0 | 16:0 | 16:3 | 18:3 |
|---|---|---|---|---|---|---|
| | Control | 0.2 ± 0 | 0.1 ± 0 | 14.0 ± 0.2 | 8.1 ± 0.1 | 57.2 ± 0 |
| | p19 only | 0.2 ± 0 | 0.1 ± 0 | 14.9 ± 0.6 | 7.0 ± 0.8 | 53.1 ± 0.7 |
| Single-gene tests | Cinca-TE | 0.4 ± 0 | 11.3 ± 1.0 | 21.9 ± 0.7 | 5.0 ± 0.2 | 38.5 ± 1.0 |
| | Cocnu-TE1 | 0.2 ± 0 | 6.3 ± 0.6 | 35.4 ± 4.7 | 4.2 ± 1.4 | 29.9 ± 5.5 |
| | Cocnu-TE2 | 0.2 ± 0 | 7.1 ± 0.3 | 31.9 ± 2.2 | 4.7 ± 0.5 | 32.9 ± 2.8 |
| | Cocnu-TE3 | 1.2 ± 0.1 | 7.2 ± 1.3 | 19.6 ± 1.6 | 5.7 ± 0.5 | 44.8 ± 2.9 |
| | Cupla-TE | 0.2 ± 0 | 1.1 ± 0.2 | 21.8 ± 2.9 | 6.0 ± 0.6 | 48.2 ± 3.1 |
| | Cupvi-TE | 0.2 ± 0 | 0.6 ± 0.1 | 17.3 ± 1.3 | 6.4 ± 0.4 | 52.9 ± 2.1 |
| | Umbca-TE | 1.6 ± 0.1 | 1.1 ± 0.2 | 14.4 ± 0.8 | 6.5 ± 0.3 | 52.7 ± 0.1 |
| | Arath- | 0.2 ± 0 | 0.4 ± 0.5 | 17.4 ± 1.0 | 6.2 ± 0.3 | 51.4 ± 1.3 |
| | LPAAT | | | | | |
| | Cocnu- | 0.1 ± 0.1 | 0.1 ± 0 | 15.1 ± 1.5 | 6.7 ± 0.5 | 52.2 ± 4.2 |
| | LPAAT | | | | | |
| +Arath-LPAAT | Cinca-TE | 0.2 ± 0 | 7.8 ± 0.1 | 24.6 ± 0.4 | 5.3 ± 0.2 | 39.2 ± 1.5 |
| | Cocnu-TE1 | 0.2 ± 0 | 4.6 ± 1.3 | 35.3 ± 1.4 | 4.4 ± 0.7 | 32.7 ± 2.0 |
| | Cocnu-TE2 | 0.2 ± 0 | 6.1 ± 0.4 | 32.5 ± 1.8 | 4.7 ± 0.1 | 34.1 ± 0.6 |
| | Cocnu-TE3 | 0.9 ± 0.2 | 8.5 ± 0.4 | 21.4 ± 1.9 | 5.6 ± 0.2 | 41.7 ± 0.6 |
| | Cupla-TE | 0.2 ± 0 | 1.0 ± 0.1 | 23.4 ± 2.7 | 5.9 ± 0.5 | 47.3 ± 1.2 |
| | Cupvi-TE | 0.2 ± 0 | 0.6 ± 0 | 19.0 ± 0.2 | 6.3 ± 0.1 | 51.4 ± 1.0 |
| | Umbca-TE | 1.2 ± 0.2 | 1.1 ± 0.1 | 15.4 ± 0.2 | 6.5 ± 0.2 | 52.3 ± 1.3 |
| +Cocnu-LPAAT | Cinca-TE | 0.7 ± 0.2 | 14.9 ± 1.6 | 23.0 ± 3.7 | 4.8 ± 1.4 | 35.4 ± 3.3 |
| | Cocnu-TE1 | 0.2 ± 0 | 5.4 ± 0.9 | 40.2 ± 2.8 | 3.3 ± 0 | 27.8 ± 1.1 |
| | Cocnu-TE2 | 0.2 ± 0 | 6.6 ± 1.0 | 38.3 ± 1.1 | 3.7 ± 0.2 | 28.2 ± 1.1 |
| | Cocnu-TE3 | 2.0 ± 0.3 | 10.9 ± 1.0 | 24.4 ± 1.8 | 4.9 ± 0.5 | 37.7 ± 0.9 |
| | Cupla-TE | 0.5 ± 0.1 | 1.6 ± 0.3 | 22.2 ± 0.6 | 6.0 ± 0.3 | 46.9 ± 2.0 |
| | Cupvi-TE | 0.5 ± 0 | 1.1 ± 0 | 19.6 ± 0.8 | 6.0 ± 0.2 | 49.8 ± 0.3 |
| | Umbca-TE | 3.3 ± 0.5 | 1.2 ± 0.1 | 13.9 ± 0.4 | 6.4 ± 0.2 | 51.3 ± 1.7 |

Co-infiltration of the chimeric gene for expressing Arath-PLPAAT with the thioesterases tended to reduce the accumulation of both C12:0 and C14:0 compared to the absence of the LPAAT, whilst slightly increasing the accumulation of C16:0. In contrast, co-infiltration of the genes for expressing Cocnu-LPAAT or Umbca-TE increased the accumulation of C12:0 to 3.3%±0.5 whilst C14:0 was found to accumulate to 14.9%±1.6 in the Cinca-TE+Cocnu-LPAAT sample. The highest C16:0 levels were observed after co-expression of Cocnu-TE1 and Cocnu-LPAAT (40.2%±2.8). Addition of an LPAAT to each inoculated zone decreased the degree of necrosis of the leaf tissue. Surprisingly, both C8:0 and C10:0 fatty acids were also produced in the plant cells in the transient expression studies. The accumulation of C8:0 and C10:0 was not observed when the thioesterase was expressed alone. However, when thioesterase expression was combined with the co-expression of CuphoFatB with CnLPAAT and AtWRI1, C8:0 was found to be present at a concentration of 0.27±0.09% of the total fatty acid content in the plant cells. Similarly, when CuplaFatB was co-expressed with CnLPAAT and AtWRI1, C10:0 was found to be present at 0.54±0.16% of the total fatty acid content.

These results indicated that the previously-reported acyl specificities of the thioesterases, observed from seed expression, were essentially maintained in N. benthamiana leaves and that this expression system was a valid system for testing acyl specificity. The addition of the plastidial A. thaliana PLPAAT did not increase the accumulation of MCFAs although it did result in slightly increased accumulation of C16:0 in A. thaliana cells. In contrast, the C. nucifera LPAAT increased the accumulation of C12:0, C14:0 and C16:0 in N. benthamiana leaves, which fatty acids are found in C. nucifera oil (Laureles et al., 2002). This indicated that the native N. benthamiana LPAAT was either not highly expressed in leaf tissue or did not have high activity on C12:0, C14:0 and C16:0 substrates.

### Medium-Chain Fatty Acid Production in Vegetative Plant Cells Accumulating High Levels of TAG

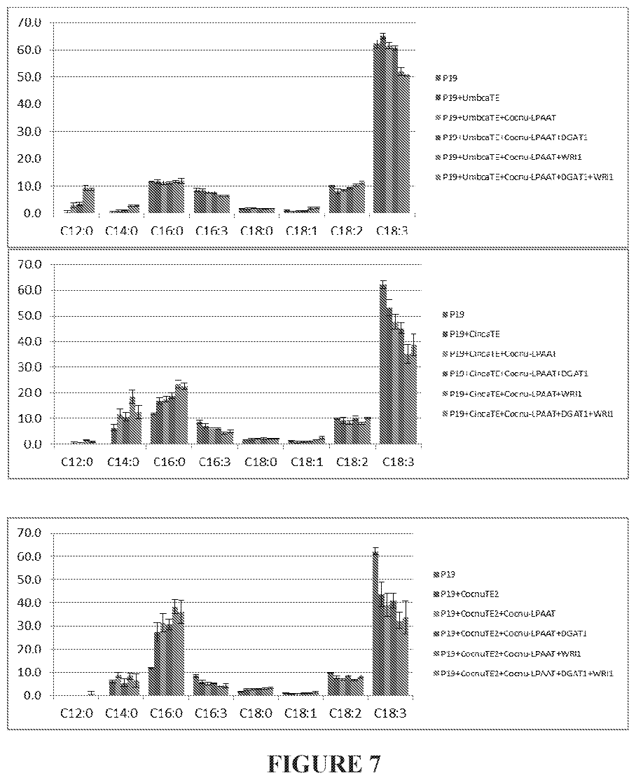

The inventors previously obtained the production of 15% TAG in N. tabacum leaves by the coordinate expression of chimeric genes encoding A. thaliana WRI1, A. thaliana DGAT1 and S. indicum Oleosin (Vanhercke et al., 2014a and b). To test whether the accumulation of MCFA that was observed after expression of thioesterases in combination with an LPAAT would also occur or be increased in plant cells producing high levels of TAG (Vanhercke et al., 2013), these genes were co-expressed. The best performing C12:0, C14:0 and C16:0 thioesterase/LPAAT combinations (Cocnu-LPAAT plus Umbca-TE, Cinca-TE and Cocnu-TE2 thioesterases, respectively) were infiltrated with and without the Arath-WRI1+DGAT combinations previously described (Vanhercke et al., 2013). The data are shown in FIG. 7.

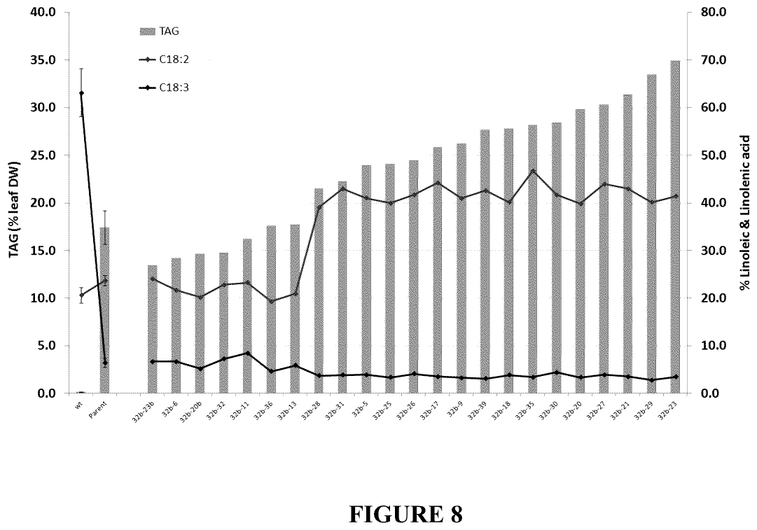

The accumulation of the relevant MCFA (C12:0 for Umbca-TE, C14:0 for Cinca-TE and C16:0 for Cocnu-TE2) was consistently and substantially increased most by the addition of Arath-WRI1 to the combinations: C12:0 comprised 9.5%±0.9 of total leaf fatty acids in the Umbca-TE+Cocnu-LPAAT+Arath-WRI1 samples, the C14:0 level was 18.5%±2.6 in the Cinca-TE+Cocnu-LPAAT+Arath-WRI1 samples and the C16:0 level was 38.3%±3.0 in the Cocnu-TE2+Cocnu-LPAAT+Arath-WRI1 samples. Thioesterase plus Arath-WRI1 infiltrations were found to have a significantly greater effect on C12:0 in the presence of Umbca-TE, C14:0 in the presence of Cinca-TE and C16:0 in the presence of Cocnu-TE2 relative to infiltration with thioesterase plus Cocnu-LPAAT in the absence of WRI1 (FIG. 8). The addition of the Cocnu-LPAAT to the thioesterase plus Arath-WRI1 mixtures did have an effect on the fatty acid composition with relatively small increases in C12:0 and C14:0 observed in the Umbca-TE and Cinca-TE sets and a small decrease in C16:0 in the Cocnu-TE2 set. The maximum levels observed were: 8.8%±1.1 of C12:0 in total leaf fatty acids observed in the Umbca-TE+Arath-WRI1+Cocnu-LPAAT samples, 14.1%±3.5 of C14:0 in the Cinca-TE+Arath-WRI¹⁴Cocnu-LPAAT samples and 48.6%±3.7 of C16:0 in the Cocnu-TE2+Arath-WRI1 sample.

Interestingly, the only thioesterase in which the Arath-WRI1 did not increase MCFA accumulation as much was the Cocnu-TE2, although it still increased significantly. The addition of this gene alone resulted in the increased accumulation of C16:0 from 16.0%±0.4 to 37.3%±0.6 whereas the further addition of Arath-WRI1 only increased this to 48.6%±1.7. This may have been due to the C12:0 and C14:0 intermediates being relatively transient during plastidial fatty acid synthesis compared to C16:0.

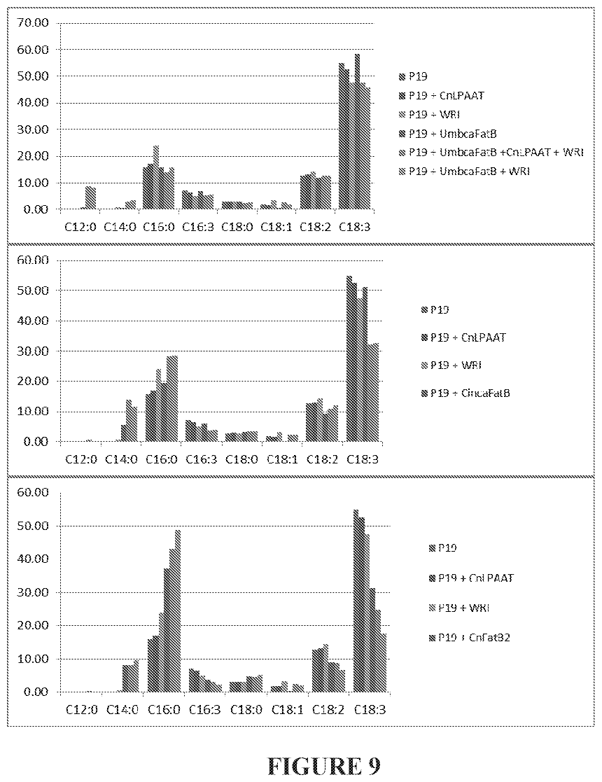

Other effects that were noted included the increase in C16:0 and C18:119 and decrease in C18:3^{Δ9,12,15} levels in the presence of Arath-WRI1. The further addition of the Cinca-TE and Cocnu-TE2 decreased C18:3^{Δ9,12,15} levels further still. In contrast, the extra C12:0 produced following the addition of Arath-WRI1 to Umbca-TE appeared to come at the cost of C16:0 rather than additional C18:3^{Δ9,12,15} (FIG. 9).

A subset of samples were also analysed by LC-MS to gain a better understanding of MCFA accumulation. The plastidial galactolipids monogalactosyl diacylglycerol (MGDG) and digalactosyl diacylglycerol (DGDG) contained only low levels of C12:0 and C14:0 and reduced levels of C16:0 relative to the p19 control infiltration. The major C12:0-containing MGDG species in the Umbca-TE samples was 30:3 indicating that one C18:3 and one C12:0 were co-located on the monogalactosyl backbone. The other main C12:0-containing MGDG species was 28:0, indicating that the second fatty acid was C16:0. The major C14:0-containing MGDG species in the Cinca-TE samples were 28:0 and 30:0, indicating that a significant proportion of the C14:0 in MGDG was either di-C14:0 or with C16:0. The C12:0-containing and C14:0-containing MGDG species were not detected in the p19 control sample. In contrast, C16:0-containing MGDG species tended to be reduced in the Cocnu-TE2 samples. The major MGDG species in the wildtype samples (C16:3-containing 34:6, C18:3-containing 34:6, and C18:3-containing 36:6) all tended to be reduced by the expression of the transgenes. This reduction was greatest in the presence of the WRI+DGAT combination.

Only trace levels of C12:0-containing DGDG species were observed in the Umbca-TE samples. The major C14:0-containing species observed in the Cinca-TE samples were 28:0 and 30:0, both of which were absent in the control. These species were also observed at elevated levels in the Cocnu-TE2 samples but only at trace levels in the Umbca-TE samples. The major DGDG species in the wildtype samples (C16:0-containing 34:3, C18:3-containing 34:3, and C18:3-containing 36:6) all tended to be reduced by the expression of the transgenes. This reduction was greatest in the presence of WRI.

Similarly, TAG species were generally increased considerably in all the samples containing WRI+DGAT as previously described (Vanhercke et al., 2013). C12:0 species were found to be dominant in the high TAG Umbca-TE sample, C14:0 in the high TAG Cinca-TE sample and C16:0 in the high TAG Cocnu-TE2 sample. LC-MS analysis of the TAG fraction showed that the C12:0-containing 36:0 was found to be the dominant TAG species, twice the level of TAG species containing C18:3, in all Umbca-TE samples containing the WRI transcription factor. Similarly, C14:0-containing 42:0 was the dominant TAG species in the Cinca-TE samples co-transformed with either LPAAT, DGAT, WRI or WRI+DGAT, although the response was considerably higher in the case of the samples containing WRI. Several C16:0-containing TAG species were significantly elevated in both the high TAG Cinca-TE (e.g. 44:0 and 50:3) and Cocnu-TE2 (e.g. 46:0, 48:0, 50:2 and 50:3) samples. Again, the greatest C16:0 increases were observed in the presence of WRI.

### Stable Transformation for Production of MCFA in Vegetative Tissues.

A series of genetic constructs were made in a binary vector in order to stably transform plants such as tobacco with combinations of genes for production of MCFA in vegetative tissues, to identify optimal combinations of genes. These constructs included a gene for expression of WRI1 under the control of either the SSU promoter (see Example 3, pOIL 121) or the senescence-specific SAG12 promoter, a gene encoding an oil palm DGAT (below), a gene encoding the coconut LPAAT (CocnuLPAAT, see above) under the control of an enTCUP promoter and several genes expressing a variety of fatty acyl thioesterases (FATB) expressed from either a 35S promoter or a SAG12 promoter. These are described below.

Cloning of a Gene Encoding Elaeis guineensis (Oil Palm) DGAT

In order to firstly test different DGAT enzymes, including representative DGAT1, DGAT2 and DGAT3 enzymes, candidate oil palm DGAT sequences were identified from the published transcriptome (Dussert et al., 2013) and codon optimised for expression in Nicotiana tabacum. The protein coding regions were then each cloned individually into binary expression vectors under the control of the 35S promoter for testing in transient N. benthamiana leaf assays as described in Example 1. The gene combinations tested were as follows:
1 P19 (negative control)
2 P19+CnLPAAT+WRI1
3 P19+CnLPAAT+AtWRI1+AtDGAT1
4 P19+CnLPAAT+AtWRI1+EgDGAT1
5 P19+CnLPAAT+AtWRI1+EgDGAT2
6 P19+CnLPAAT+AtWRI1+EgDGAT3
7 P19+CincaFatB
8 P19+CincaFatB+CnLPAAT+WRI1
9 P19+CincaFatB+CnLPAAT+AtWRI1+AtDGAT1
10 P19+CincaFatB+CnLPAAT+AtWRI1+EgDGAT1
11 P19+CincaFatB+CnLPAAT+AtWRI1+EgDGAT2
12 P19+CincaFatB+CnLPAAT+AtWRI1+EgDGAT3

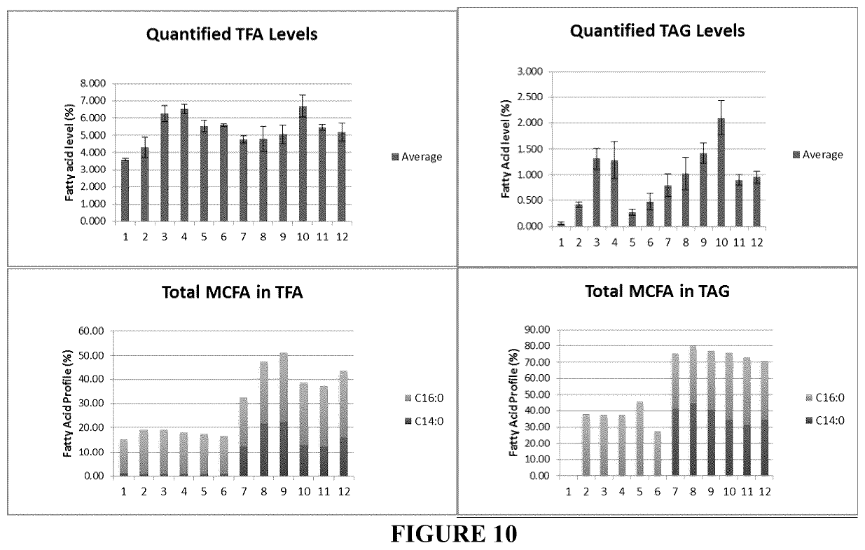

The results for the TFA and TAG levels, and the levels of total MCFA in the TFA or the TAG contents, are shown in FIG. 10. Compared to AtDGAT1, the expression of EgDGAT1 led to greater accumulation of total fatty acids and increased TAG levels. The total MCFA content in the total fatty acid content was reduced with the expression of EgDGAT1 relative to AtDGAT1, but the levels of MCFA present in TAG remained about the same (FIG. 10).

### Preparation of Genetic Constructs

Genetic constructs for stable transformation (Table 12) were assembled through the sequential insertion of gene cassettes through the use of compatible restriction enzyme sites. The four gene constructs (Table 12) each contained a gene encoding the oil palm DGAT1 (EgDGAT1) expressed from the 35S promoter, a gene encoding the C. nucifera LPAAT (CnLPAAT) expressed from the constitutive enTCUP2 promoter, and a gene encoding AtWRI1 expressed from either the SSU promoter or the SAG12 promoter in addition to one of a series of genes encoding FATB enzymes.

The five gene constructs also contained a gene for expression of a hairpin RNA for reducing expression of an endogenous gene encoding acyl-activating enzyme (AAE). The hairpin was constructed based on sequence similarity with the identified AAE15 from Arabidopsis lyrata (EFH44575.1) and the N. benthamiana genome. AAE has been shown to be involved in the reactivation of MCFA, and hence further elongation. It was considered that silencing of AAE might increase MCFA accumulation. The hairpin cassette was constructed in the vector pKANNIBAL and then subcloned into the expression vector pWBVec2 with the expression of the hairpin being driven by the 35S promoter.

TABLE 12
Summary of assembled genetic constructs.

| | Construct | Gene Combination |
|---|---|---|
| Single Gene | pKR1 | 35S::UmbcaFATB |
| Constructs | pKR2 | 35S::CincaFATB |
| | pKR3 | 35S::CocnuFATB2 |
| | pOIL115 | SAG12::CincaFATB |
| | pOIL116 | SAG12::UmbcaFATB |
| | pOIL117 | SAG12::CocnuFATB2 |
| Construction | pOIL300 | 35S::EgDGAT1 |
| Components | pOIL301 | enTCUP::CnLPAAT inFATBrmediaFATB construct |
| | pOIL302 | 35S::EgDGAT1 + enTCUP::CnLPAAT |
| | pOIL303 | 35S::EgDGAT1 + enTCUP::CnLPAAT + SSU:AtWRI1 |
| | pOIL304 | 35S::EgDGAT1 + enTCUP::CnLPAAT + SAG12:AtWRI1 |
| Four Gene | pOIL305 | 35S::EgDGAT1 + enTCUP::CnLPAAT + SSU:AtWRI1 + |
| Constructs | | 35S::UmbcaFATB |
| | pOIL306 | 35S::EgDGAT1 + enTCUP::CnLPAAT + SSU:AtWRI1 + |
| | | 35S::CincaFATB |
| | pOIL307 | 35S::EgDGAT1 + enTCUP::CnLPAAT + SSU:AtWRI1 + |
| | | 35S::CocnuFATB2 |
| | pOIL308 | 35S::EgDGAT1 + enTCUP::CnLPAAT + SSU:AtWRI1 + |
| | | SAG12::UmbcaFATB |
| | pOIL309 | 35S::EgDGAT1 + enTCUP::CnLPAAT + SSU:AtWRI1 + |
| | | SAG12::CincaFATB |
| | pOIL310 | 35S::EgDGAT1 + enTCUP::CnLPAAT + SSU:AtWRI1 + |
| | | SAG12::CocnuFATB2 |
| | pOIL311 | 35S::EgDGAT1 + enTCUP::CnLPAAT + SAG12:AtWRI1 + |
| | | 35S::UmbcaFATB |
| | pOIL312 | 35S::EgDGAT1 + enTCUP::CnLPAAT + SAG12:AtWRI1 + |
| | | 35S::CincaFATB |
| | pOIL313 | 35S::EgDGAT1 + enTCUP::CnLPAAT + SAG12:AtWRI1 + |
| | | 35S::CocnuFATB2 |
| | pOIL314 | 35S::EgDGAT1 + enTCUP::CnLPAAT + SAG12:AtWRI1 + |
| | | SAG12::UmbcaFATB |
| | pOIL315 | 35S::EgDGAT1 + enTCUP::CnLPAAT + SAG12:AtWRI1 + |
| | | SAG12::CincaFATB |
| | pOIL316 | 35S::EgDGAT1 + enTCUP::CnLPAAT + SAG12:AtWRI1 + |
| | | SAG12::CocnuFATB2 |
| Five Gene | pOIL317 | 35S::EgDGAT1 + enTCUP::CnLPAAT + SSU:AtWRI1 + |
| Constructs | | 35S::UmbcaFATB + 35S::hpNbAAE |
| | pOIL318 | 35S::EgDGAT1 + enTCUP::CnLPAAT + SSU:AtWRI1 + |
| | | 35S::CincaFATB + 35S::hpNbAAE |
| | pOIL319 | 35S::EgDGAT1 + enTCUP::CnLPAAT + SSU:AtWRI1 + |
| | | 35S::CocnuFATB2 + 35S::hpNbAAE |
| | pOIL320 | 35S::EgDGAT1 + enTCUP::CnLPAAT + SSU:AtWRI1 + |
| | | SAG12::UmbcaFATB + 35S::hpNbAAE |
| | pOIL321 | 35S::EgDGAT1 + enTCUP::CnLPAAT + SSU:AtWRI1 + |
| | | SAG12::CincaFATB + 35S::hpNbAAE |
| | pOIL322 | 35S::EgDGAT1 + enTCUP::CnLPAAT + SSU:AtWRI1 + |
| | | SAG12::CocnuFATB2 + 35S::hpNbAAE |
| | pOIL323 | 35S::EgDGAT1 + enTCUP::CnLPAAT + SAG12:AtWRI1 + |
| | | 35S::UmbcaFATB + 35S::hpNbAAE |
| | pOIL324 | 35S::EgDGAT1 + enTCUP::CnLPAAT + SAG12:AtWRI1 + |
| | | 35S::CincaFATB + 35S::hpNbAAE |
| | pOIL325 | 35S::EgDGAT1 + enTCUP::CnLPAAT + SAG12:AtWRI1 + |
| | | 35S::CocnuFATB2 + 35S::hpNbAAE |
| | pOIL326 | 35S::EgDGAT1 + enTCUP::CnLPAAT + SAG12:AtWRI1 + |
| | | SAG12::UmbcaFATB + 35S::hpNbAAE |
| | pOIL327 | 35S::EgDGAT1 + enTCUP::CnLPAAT + SAG12:AtWRI1 + |
| | | SAG12::CincaFATB + 35S::hpNbAAE |
| | pOIL328 | 35S::EgDGAT1 + enTCUP::CnLPAAT + SAG12:AtWRI1 + |
| | | SAG12::CocnuFATB2 + 35S::hpNbAAE |

These genetic constructs were used to produce transformed tobacco plants of cultivars Wisconsin 38 and a high oil line transformed with the T-DNA from pJP3502. It was observed that plants transformed with the single gene FATB constructs expressed from the 35S promoter were significantly smaller than those transformed with the corresponding FATB construct expressed from the SAG12 promoter or from the four gene constructs. The smaller plant size was considered to be caused by a buildup of MCFA which was not incorporated efficiently into TAG.

### Discussion

The present study found that C12:0 production in leaf cells was only about 1.6% of the total fatty acid content after expression of Umbca-TE alone (Table 11). The addition of a gene for expression of Arath-WRI had a much stronger effect on C12:0 and C14:0 accumulation in leaf tissue than the addition of the coconut LPAAT (FIGS. 7 and 9). This indicated that WRI1 in combination with the thioesterase greatly increased MCFA accumulation in leaf cells, acting synergistically. Importantly, much of the C12:0, C14:0 and C16:0 was found to accumulate in the leaves in TAG, which lipid does not accumulate at substantial levels in wild-type leaves. These experiments showed that the cells in the vegetative parts of plants could be modified to produce MCFA, particularly C12:0 and C14:0 in TAG at high levels. C16:0 levels were also increased substantially.

### Example 11: Gene Selection and Vector Construction

Fatty acyl thioesterases were identified from Cinnamomum camphora 14:0-ACP thioesterase (referred to as ‘CcTE’, Accession No. Q39473.1, (Yuan et al., 1995)), Umbellularia californica 12:0-ACP thioesterase (UcTE, Accession No. Q41635.1, (Voelker et al., 1992)), and Cocos nucifera acyl-ACP thioesterase FatB2 (CnTE2, Accession No. AEM72520.1, (Jing et al., 2011)). A C. nucifera LPAAT (CnLPAAT, Accession No. Q42670.1, (Knutzon et al., 1995)) was also identified. Coding regions were synthesized using codon optimised nucleotide sequences for expression in Nicotiana plant cells. Expression vectors encoding WRI1 and DGAT were produced as previously described by Vanhercke et al. (2013).

Three DGAT candidate sequences were identified in the transcriptome of African oil palm (Elaeis guineensis) (Dussert et al., 2013) and selected to be tested in their utilisation of MCFA for the assembly of leaf lipids. The DGATs from oil palm were selected based on the fatty acid compositions of palm oil and palm kernel oil (Edem, 2002), being high in MCFA content.

A gene encoding glycerol-3-phosphate acyltransferase 9 (GPAT9) from C. nucifera (coconut, (nGPAT9) was identified from a transcriptome. A genetic construct to express this enzyme was made from RNA isolated from developing coconut endosperm, as described below.

Each gene was cloned into the EcoRI site of the binary vector pJP3343 which contained a constitutive 35S promoter with duplicated enhancer region (Vanhercke et al., 2013) for expression in plant cells. Agrobacterium tumefaciens strain AGL1 was transformed with each of the constructs.

### Example 12: Increasing Medium Chain Fatty Acid Production in Vegetative Plant Cells

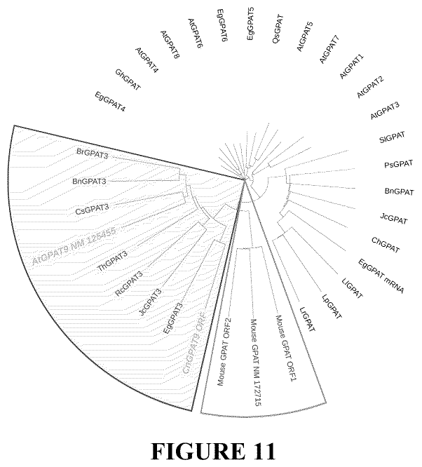

GPAT9 has recently been identified as functioning in Arabidopsis thaliana seed to transfer acyl groups from acyl-CoA to the sn-1 position of glycerol-3-phosphate (G3P) (Shockey et al., 2016; Singer et al., 2016). The inventors hypothesized that a GPAT9 from coconut might assist in increasing the MCFA content of transgenic oils produced in vegetative plant cells. A GPAT9 gene from coconut was identified by searching an assembled coconut endosperm transcriptome using the Arabidopsis thaliana GPAT9 nucleotide sequence (AtGPAT9) (Shockey et al., 2016) as the BLAST query. A candidate for GPAT9 from coconut was identified, namely NCBI Accession number KX235871. High fidelity PCR was used to amplify the full length CnGPAT9 cDNA sequence from coconut. Following isolation and sequencing of the full length transcript of interest, the open reading frame for the predicted CnGPAT9 was identified. The predicted amino acid sequence was aligned with the sequence of AtGPAT9, revealing that the sequences were 78% identical. Sequence alignment with other annotated GPAT nucleotide sequences showed that the identified CnGPAT9 nucleotide sequence clustered with other GPAT9 sequences (FIG. 11).

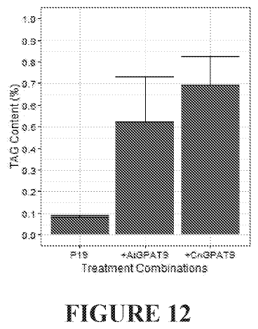

A nucleotide sequence encoding the candidate CnGPAT9 was synthesized and inserted into pJP3343 in order to test its enzymatic function using the transient N. benthamiana infiltration assay as described in Example 1, in particular to test its ability to increase TAG content. AtGPAT9 was used as a positive control. Total lipids were extracted from infiltrated leaf zones and analysed to determine the effect of the GPAT9s on TAG content (FIG. 12). From comparison with the samples where p19 alone was infiltrated, which provided a TAG level of about 0.1%, expression of either AtGPAT9 or CnGPAT9 provided significant increases in the TAG content in the leaf, to 0.5±0.2% and 0.7±0.1% on a dry weight basis, respectively. There was no significant difference in the TAG levels between the two GPAT9s. It was concluded from these data and the phylogeny (FIG. 11) that the isolated CnGPAT9 sequence from coconut encoded a functional GPAT9.

### Example 13: DGAT1 Promotes Production of MCFA-Enriched Oils

It has been previously demonstrated that MCFA-containing oils could be produced in the leaves of N. benthamiana (Reynolds et al., 2015). However, chlorosis of the leaves was observed with some gene combinations when MCFA accumulated in membrane lipids such as PC. The inventors wanted to test whether the introduction of a DGAT capable of esterifying MCFA into TAG might increase the MCFA content and perhaps reduce the chlorosis phenotype.

Gene candidates that might be involved in lipid synthesis pathways were identified in the Elaeis guineensis (African oil palm) transcriptome (Dussert et al., 2013) as described above. The fatty acid profile of the oils from oil palm (palm oil and palm kernel oil) (Edem, 2002) suggested that some DGATs from oil palm might exhibit preference for MCFA substrates. Sequences for three candidate DGAT1 cDNAs were identified from the E. guineensis transcriptome. Alignment of the predicted amino acid sequences after translation of the cDNAs revealed that the isoforms designated EgDGAT1.2 and EgDGAT1.3 lacked highly conserved C- and N-terminal motifs (Cao, 2011) which are responsible for the catalytic and regulatory activities of DGAT1, respectively (Liu et al., 2012; Xu et al., 2008), suggesting these isoforms would be non-functional. The third candidate EgDGAT1.1 had these conserved motifs and was further tested.

A genetic construct with codon optimization for expressing EgDGAT1.1 in N. tabacum was synthesized and infiltrated into N. benthamiana in combination with genetic constructs to express Arabidopsis thaliana WRI1 and CnLPAAT. The infiltrations were either with or without a gene for co-expression of a thioesterase from Cinnamomum camphora (CcTE), to measure levels of both TAG production and the incorporation of MCFA into TAG. Five days after infiltration, a strong chlorosis phenotype was observed to be associated with several gene combinations, correlated in particular with the presence of CcTE. Surprisingly, the chlorosis phenotype was alleviated by the addition of the gene encoding EgDGAT1.1 (hereinafter referred to as EgDGAT1) more so than with AtDGAT1. It was hypothesized that the alleviation of the negative chlorosis phenotype was due to the increased capacity of EgDGAT1 to sequester MCFA into TAG relative to AtDGAT1.

Total lipids were extracted and analysed in order to better understand the relationship between chlorosis and the particular gene combinations. The total fatty acid profile revealed that in the absence of CcTE, the TFA content was similar in the presence of either AtDGAT1 or EgDGAT1. In the presence of CcTE, the TFA content was significantly greater for treatments including EgDGAT1 relative to AtDGAT1. The same correlation was observed for TAG content. Although the TAG content was similar for the AtWRI1+AtDGAT1 and AtWRI1+EgDGAT1.1 samples, the TAG content was significantly increased for samples expressing CcTE and EgDGAT1, compared to samples expressing AtDGAT1. These results suggested that following CcTE expression, in the presence of AtDGAT1, fatty acid synthesis was inhibited due to inefficient assembly of the MCFA into glycerolipids. Conversely, there appeared to be no inhibition of fatty acid synthesis following the addition of EgDGAT highlighted by increases in both the TFA and TAG content, implying improved incorporation efficiency for MCFAs.

The fatty acid composition of the phospholipid fraction in the infiltrated leaf zones was also analysed. Total phospholipids were fractionated by TLC and prepared for analysis by the preparation of FAME. Analysis of the fatty acid composition of the phospholipids revealed a significant reduction in the accumulation of MCFA, particularly C14:0 and C16:0, following the expression of the EgDGAT1 construct, compared to AtDGAT1. This suggested that the reduced accumulation of MCFA into membrane lipids assisted in reducing the chlorosis phenotype.

### Example 14: Reconfiguration of Kennedy Pathway for Efficient MCFA Accumulation

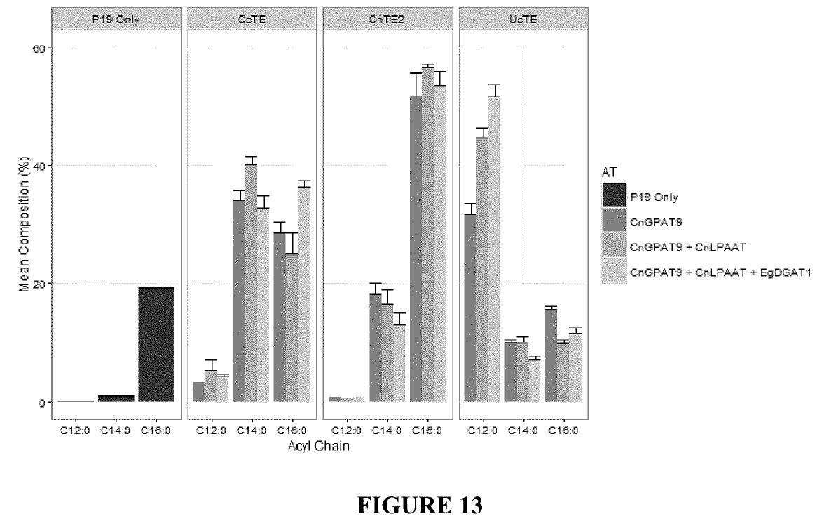

Following confirmation of CnGPAT9 activity, its capability to use various MCFA acyl-CoAs as substrates for TAG assembly was tested. This was done in the context of the Kennedy pathway components LPAAT and DGAT1, as well as WRI1 to increase the level of fatty acid synthesis. The fatty acid composition of TAG and the TAG content were determined by GC-FID (FIG. 13, Tables 13-15). When combined with co-expression of UcTE, the sequential addition of each acyltransferase resulted in both significantly increased total TAG content, and a significantly increased accumulation of laurate (C12:0) in the TAG as a percentage of the total fatty acid content of the TAG. C12:0 levels were up to 51.6±2.0% in the presence of the combined expression of UcTE+AtWRI1+CnGPAT9+CnLPAAT+EgDGAT1, at a total TAG content in the leaf tissue of 2.4±0.7%. It was also observed that this combination was associated with a reduction of the chlorosis phenotype, thought by the inventors to be a result of efficient sequestering of laurate into TAG, i.e. less inclusion in membrane lipids such as PC. Similar results were observed with the co-expression of CcTE. C14:0 accumulated to 40.3±1.2% in the presence of the combination of CcTE+AtWRI1+CnGPAT9+CnLPAAT. There was an increase in the TAG content but not significantly compared to CcTE+CnGPAT9. The greatest TAG production was achieved following the further addition of the EgDGAT1, with a total TAG content of 2.8±0.2%. The fatty acid composition of TAG was altered following the additional combination with EgDGAT1, with a significant reduction in C14:0 and a significant increase in C16:0 content, each as a percentage of the total fatty acid content of the TAG. This shift in profile suggested that EgDGAT1 exhibited a stronger substrate preference for C16:0 compared to C14:0. Consistent with the observations with UcTE, a significant improvement in the chlorotic phenotype was observed following the addition of EgDGAT1. When CnTE2 was used, the sequential addition of the acyltransferases did not result in any significant differences in either the fatty acid profile of TAG, or the total TAG content. This may have been due to the native acyltransferases' ability to efficiently utilise the increased flux of C16:0 acyl-CoA associated with the activity of CnTE2.

Further investigations into the effects of the sequential addition of acyltransferases on the utilization of acyl-CoAs for the assembly of MCFA-enriched glycerolipids was performed using QQQ-LCMS as described in Example 1, to reveal any differences in MCFA assembly and distribution. The integrated analysis including DAG, PC and TAG revealed much information about the assembly process of lipids in the leaf cells. When CnGPAT9 was expressed with UcTE+AtWRI1, it was observed that CnGPAT9 used C12:0 substrate for assembly, based on the presence of PC 30:3 (C12:0 plus C18:3). It was reasoned that the sn-2 position of the PC was most likely occupied by C18:3, due to either the esterification of C12:0 to the sn-1 position via CnGPAT9 or from the absence of CnLPAAT. The presence of some TAG 42:3 suggested that the native DGATs exhibited some capability of utilising C12:0 for TAG assembly (12:0/18:3/12:0). With the addition of CnLPAAT, a significant amount of PC 24:0 (di-C12:0) was produced, indicating that C12.0 was efficiently esterified to both the su-1 and sn-2 positions of the G3P backbone. However, without a strong substrate preference for C12:0, most of the produced laurate remains sequestered in membrane lipids. However, further addition of EgDGAT1 increased laurate accumulation. This shift involved the reduction of MCFAs accumulating in PC and increased production of MCFA-enriched TAG. Most notable was the shift from PC 24:0 (without EgDGAT1) to the accumulation of TAG 36:0 (tri-C12:0) (with EgDGAT1), highlighting that laurate was being efficiently incorporated into all three position of the G3P backbone in the presence of EgDGAT1. Significant increases were also observed for other MCFA-enriched TAG species including TAG 38:0, TAG 40:0 and TAG 42.0. These results confirmed that the expression of an appropriate DGAT1 was effective for the efficient incorporation of the unusual fatty acids of interest (in this instance, C12:0 and other MCFA) into TAG. These results highlighted that the expression of the EgDGAT1 in the enzyme combination effectively relieved the accumulation of MCFA in PC and promoted efficient production of MCFA-enriched TAG in plant leaf lipids.

A similar pattern was also observed in the case study involving combinations including CcTE. When CnGPAT9 was combined with CcTE+AtWRI1, it was observed that CnGPAT9 utilised C14:0 substrate, based on the accumulation of PC 28:0 (di-C14:0) and PC 30:0 (C14:0 plus C16:0). It appeared that the native LPAAT genes were somewhat capable of utilising C14:0-CoA as substrate based on the presence of PC 28:0, indicating that C14:0 was being esterified at both the sn-1 and sn-2 positions of the PC. Similarly, the native DGATs also appeared capable of utilising C14:0-CoA for TAG assembly, based on the production of TAG 42:0 (tri-C14:0). However, the subsequent addition of CnLPAAT to the system increased utilisation of C14:0 acyl-CoA, evident from the significantly increased abundance of PC 28:0, which indicated an increased efficiency of esterification to the sn-2 position of PC. This increased accumulation of MCFA was also correlated with a more severe chlorosis phenotype then compared to the CnGPAT9 alone, most likely attributed to the increased accumulation in the membrane lipids. The further addition of the EgDGAT1 to the combination resulted in almost complete absence of MCFA from PC. This was associated with an increased production of MCFA-enriched TAG species, particularly TAG 40:0, TAG 42:0, TAG 44:0 and TAG 46:0, all of which include the incorporation of C14:0.

When CnGPAT9 was combined with CnTE2+AtWRI1, it was observed that CnGPAT9 also utilised C16:0-CoA as substrate, based on the accumulation of PC 32:0 (di-C16:0). Based on the fatty acid profile of N. benthamiana leaves, it was expected that the native LPAATs and DGATs would exhibit substrate preference for the incorporation of C16:0 into glycerolipids. evidenced from the increased production of C16:0-enriched TAG species, through simply over-expressing a thioesterase with C16:0 specificity. Although the subsequent additions of the CnLPAAT and EgDGAT1 did not appear to significantly affect the overall TAG composition, there was a significant reduction in the total MCFA accumulation in PC lipids. Importantly, the addition of the EgDGAT1 to CnTE2 was associated with a reduction in the degree of leaf chlorosis, although not as complete as in the presence of the other TEs.

It was concluded that a GPAT9 like CnGPAT9 having a preference for MCFA substrates was an important factor in contributing towards both MCFA accumulation and increasing the total production of TAG in plant leaves. In the absence of a DGAT having substrate preference for MCFA, the low abundance of MCFA-containing DAG species suggested that DAG containing the MCFA was efficiently converted to PC through the activities of either PDCT or CPT (Bates and Browse, 2011; Bates and Browse, 2012; Bates et al., 2012). The addition of EgDGAT1 changed the metabolic flux of the system, pushing MCFA towards TAG accumulation via the Kennedy pathway, and thus away from incorporation of the MCFA into membrane lipids through reducing conversion of DAG to PC.

TABLE 13
Total leaf fatty acid composition of TAG (% total TAG) of C6:0, C8:0 and C10:0 fatty
acids in Nicotiana benthamiana leaves infiltrated with various constructs.

| | | | |
|---|---|---|---|
| Genotype | C6:0 | C8:0 | C10:0 |

REPLICATE 1

| | | | |
|---|---|---|---|
| P19 | 0.000 | 0.000 | 4.317 |
| P19 + CincaTE + CnGPAT9 + AtWRI | 0.000 | 0.000 | 0.000 |
| P19 + CuplaTE + CnGPAT9 + AtWRI | 0.000 | 0.000 | 5.687 |
| P19 + UmbcaTE + CnGPAT9 + AtWRI | 0.000 | 0.000 | 0.000 |
| P19 + CocnuTE2 + CnGPAT9 + AtWRI | 0.000 | 0.000 | 0.000 |
| P19 + CincaTE + CnGPAT9 + AtWRI + CnLPAAT | 0.000 | 0.000 | 0.000 |
| P19 + CuplaTE + CnGPAT9 + AtWRI + CnLPAAT | 0.000 | 0.000 | 0.947 |
| P19 + UmbcaTE + CnGPAT9 + AtWRI + CnLPAAT | 0.000 | 0.000 | 0.000 |
| P19 + CocnuTE2 + CnGPAT9 + AtWRI + CnLPAAT | 0.000 | 0.000 | 0.000 |
| P19 + CincaTE + CnGPAT9 + AtWRI + CnLPAAT + EgDGAT | 0.000 | 0.000 | 0.000 |
| P19 + CuplaTE + CnGPAT9 + AtWRI + CnLPAAT + EgDGAT | 0.000 | 0.000 | 1.533 |
| P19 + UmbcaTE + CnGPAT9 + AtWRI + CnLPAAT + EgDGAT | 0.000 | 0.000 | 0.000 |
| P19 + CocnuTE2 + CnGPAT9 + AtWRI + CnLPAAT + EgDGAT | 0.000 | 0.000 | 0.000 |
| P19 + CincaTE + AtGPAT9 + AtWRI + CnLPAAT + EgDGAT | 0.000 | 0.000 | 0.000 |
| P19 + CuplaTE + AtGPAT9 + AtWRI + CnLPAAT + EgDGAT | 0.000 | 0.000 | 1.643 |
| P19 + UmbcaTE + AtGPAT9 + AtWRI + CnLPAAT + EgDGAT | 0.000 | 0.000 | 0.000 |

REPLICATE 2

| | | | |
|---|---|---|---|
| P19 | 0.000 | 0.000 | 0.000 |
| P19 + CincaTE + CnGPAT9 + AtWRI | 0.000 | 0.000 | 0.000 |
| P19 + CuplaTE + CnGPAT9 + AtWRI | 0.000 | 0.000 | 4.368 |
| P19 + UmbcaTE + CnGPAT9 + AtWRI | 0.000 | 0.000 | 0.000 |
| P19 + CocnuTE2 + CnGPAT9 + AtWRI | 0.000 | 0.000 | 0.000 |
| P19 + CincaTE + CnGPAT9 + AtWRI + CnLPAAT | 0.000 | 0.000 | 0.000 |
| P19 + CuplaTE + CnGPAT9 + AtWRI + CnLPAAT | 0.000 | 0.000 | 3.523 |
| P19 + UmbcaTE + CnGPAT9 + AtWRI + CnLPAAT | 0.000 | 0.000 | 0.000 |
| P19 + CocnuTE2 + CnGPAT9 + AtWRI + CnLPAAT | 0.000 | 0.000 | 0.000 |
| P19 + CincaTE + CnGPAT9 + AtWRI + CnLPAAT + EgDGAT | 0.000 | 0.000 | 0.000 |
| P19 + CuplaTE + CnGPAT9 + AtWRI + CnLPAAT + EgDGAT | 0.000 | 0.000 | 0.000 |
| P19 + UmbcaTE + CnGPAT9 + AtWRI + CnLPAAT + EgDGAT | 0.000 | 0.000 | 0.000 |
| P19 + CocnuTE2 + CnGPAT9 + AtWRI + CnLPAAT + EgDGAT | 0.000 | 0.000 | 0.000 |
| P19 + CincaTE + AtGPAT9 + AtWRI + CnLPAAT + EgDGAT | 0.000 | 0.000 | 0.000 |
| P19 + CuplaTE + AtGPAT9 + AtWRI + CnLPAAT + EgDGAT | 0.000 | 0.000 | 0.000 |
| P19 + UmbcaTE + AtGPAT9 + AtWRI + CnLPAAT + EgDGAT | 0.000 | 0.000 | 0.000 |

REPLICATE 3

| | | | |
|---|---|---|---|
| P19 | 0.000 | 0.000 | 0.000 |
| P19 + CincaTE + CnGPAT9 + AtWRI | 0.000 | 0.000 | 0.000 |
| P19 + CuplaTE + CnGPAT9 + AtWRI | 0.000 | 0.000 | 0.000 |
| P19 + UmbcaTE + CnGPAT9 + AtWRI | 0.000 | 0.000 | 0.000 |
| P19 + CocnuTE2 + CnGPAT9 + AtWRI | 0.000 | 0.000 | 0.000 |
| P19 + CincaTE + CnGPAT9 + AtWRI + CnLPAAT | 0.000 | 0.000 | 0.000 |
| P19 + CuplaTE + CnGPAT9 + AtWRI + CnLPAAT | 0.000 | 0.000 | 0.000 |
| P19 + UmbcaTE + CnGPAT9 + AtWRI + CnLPAAT | 0.000 | 0.000 | 0.000 |
| P19 + CocnuTE2 + CnGPAT9 + AtWRI + CnLPAAT | 0.000 | 0.000 | 0.000 |
| P19 + CincaTE + CnGPAT9 + AtWRI + CnLPAAT + EgDGAT | 0.000 | 0.000 | 0.000 |
| P19 + CuplaTE + CnGPAT9 + AtWRI + CnLPAAT + EgDGAT | 0.000 | 0.000 | 0.000 |
| P19 + UmbcaTE + CnGPAT9 + AtWRI + CnLPAAT + EgDGAT | 0.000 | 0.000 | 0.000 |
| P19 + CocnuTE2 + CnGPAT9 + AtWRI + CnLPAAT + EgDGAT | 0.000 | 0.000 | 0.000 |
| P19 + CincaTE + AtGPAT9 + AtWRI + CnLPAAT + EgDGAT | 0.000 | 0.000 | 0.000 |
| P19 + CuplaTE + AtGPAT9 + AtWRI + CnLPAAT + EgDGAT | 0.000 | 0.000 | 0.000 |
| P19 + UmbcaTE + AtGPAT9 + AtWRI + CnLPAAT + EgDGAT | 0.000 | 0.000 | 0.000 |

TABLE 14
Total leaf fatty acid composition of TAG (% total TAG) of 12:0, C14:0, C14;1, C15:0, C16:0
and C16:1 fatty acids in Nicotiana benthamiana leaves infiltrated with various constructs.

| | | | | | | |
|---|---|---|---|---|---|---|
| Genotype | C12:0 | C14:0 | C14:1 | C15:0 | C16:0 | C16:1 |

REPLICATE 1

| | | | | | | |
|---|---|---|---|---|---|---|
| P19 | 3.882 | 11.116 | 0.000 | 1.380 | 41.258 | 0.000 |
| P19 + CincaTE + CnGPAT9 + AtWRI | 3.332 | 35.333 | 0.000 | 0.226 | 27.276 | 0.203 |
| P19 + CuplaTE + CnGPAT9 + AtWRI | 2.119 | 10.647 | 0.000 | 0.000 | 47.322 | 0.000 |
| P19 + UmbcaTE + CnGPAT9 + AtWRI | 32.957 | 9.794 | 0.000 | 0.000 | 16.217 | 0.000 |
| P19 + CocnuTE2 + CnGPAT9 + AtWRI | 0.000 | 17.998 | 0.000 | 0.343 | 56.230 | 0.578 |
| P19 + CincaTE + CnGPAT9 + AtWRI + CnLPAAT | 7.219 | 41.154 | 0.000 | 0.261 | 24.586 | 0.334 |
| P19 + CuplaTE + CnGPAT9 + AtWRI + CnLPAAT | 1.491 | 7.331 | 0.000 | 0.241 | 57.931 | 0.315 |
| P19 + UmbcaTE + CnGPAT9 + AtWRI + CnLPAAT | 44.742 | 10.476 | 0.000 | 0.000 | 10.207 | 0.270 |
| P19 + CocnuTE2 + CnGPAT9 + AtWRI + CnLPAAT | 0.465 | 14.889 | 0.000 | 0.335 | 56.250 | 0.481 |
| P19 + CincaTE + CnGPAT9 + AtWRI + CnLPAAT + EgDGAT | 4.620 | 30.554 | 0.000 | 0.177 | 37.511 | 0.475 |
| P19 + CuplaTE + CnGPAT9 + AtWRI + CnLPAAT + EgDGAT | 4.598 | 5.250 | 0.000 | 0.221 | 51.742 | 0.320 |
| P19 + UmbcaTE + CnGPAT9 + AtWRI + CnLPAAT + EgDGAT | 53.604 | 7.690 | 0.000 | 0.157 | 11.120 | 0.094 |
| P19 + CocnuTE2 + CnGPAT9 + AtWRI + CnLPAAT + EgDGAT | 0.654 | 14.116 | 0.000 | 0.256 | 53.942 | 0.306 |
| P19 + CincaTE + AtGPAT9 + AtWRI + CnLPAAT + EgDGAT | 4.499 | 35.151 | 0.000 | 0.196 | 33.202 | 0.314 |
| P19 + CuplaTE + AtGPAT9 + AtWRI + CnLPAAT + EgDGAT | 4.943 | 5.716 | 0.000 | 0.262 | 50.177 | 0.542 |
| P19 + UmbcaTE + AtGPAT9 + AtWRI + CnLPAAT + EgDGAT | 49.589 | 7.781 | 0.000 | 0.105 | 12.284 | 0.252 |

REPLICATE 2

| | | | | | | |
|---|---|---|---|---|---|---|
| P19 | 6.485 | 10.998 | 0.000 | 0.000 | 46.160 | 0.000 |
| P19 + CincaTE + CnGPAT9 + AtWRI | 0.000 | 0.000 | 0.000 | 0.000 | 0.000 | 0.000 |
| P19 + CuplaTE + CnGPAT9 + AtWRI | 1.758 | 10.767 | 0.000 | 0.000 | 49.728 | 0.583 |
| P19 + UmbcaTE + CnGPAT9 + AtWRI | 32.530 | 10.553 | 0.000 | 0.000 | 15.254 | 0.544 |
| P19 + CocnuTE2 + CnGPAT9 + AtWRI | 0.628 | 16.693 | 0.000 | 0.327 | 49.863 | 0.466 |
| P19 + CincaTE + CnGPAT9 + AtWRI + CnLPAAT | 3.660 | 40.701 | 0.000 | 0.264 | 28.736 | 0.333 |
| P19 + CuplaTE + CnGPAT9 + AtWRI + CnLPAAT | 2.472 | 10.374 | 0.000 | 0.364 | 49.195 | 0.635 |
| P19 + UmbcaTE + CnGPAT9 + AtWRI + CnLPAAT | 43.462 | 10.775 | 0.000 | 0.206 | 10.328 | 0.225 |
| P19 + CocnuTE2 + CnGPAT9 + AtWRI + CnLPAAT | 0.000 | 0.000 | 0.000 | 0.000 | 0.000 | 0.000 |
| P19 + CincaTE + CnGPAT9 + AtWRI + CnLPAAT + EgDGAT | 4.101 | 33.380 | 0.000 | 0.000 | 35.431 | 0.000 |
| P19 + CuplaTE + CnGPAT9 + AtWRI + CnLPAAT + EgDGAT | 8.061 | 5.606 | 0.000 | 0.000 | 47.901 | 0.000 |
| P19 + UmbcaTE + CnGPAT9 + AtWRI + CnLPAAT + EgDGAT | 49.552 | 6.800 | 0.000 | 0.000 | 12.602 | 0.000 |
| P19 + CocnuTE2 + CnGPAT9 + AtWRI + CnLPAAT + EgDGAT | 0.000 | 14.374 | 0.000 | 0.000 | 50.723 | 0.000 |
| P19 + CincaTE + AtGPAT9 + AtWRI + CnLPAAT + EgDGAT | 2.758 | 26.757 | 0.000 | 0.000 | 38.082 | 0.000 |
| P19 + CuplaTE + AtGPAT9 + AtWRI + CnLPAAT + EgDGAT | 2.672 | 4.771 | 0.000 | 0.000 | 53.725 | 0.000 |
| P19 + UmbcaTE + AtGPAT9 + AtWRI + CnLPAAT + EgDGAT | 49.847 | 6.988 | 0.000 | 0.000 | 11.945 | 0.000 |

REPLICATE 3

| | | | | | | |
|---|---|---|---|---|---|---|
| P19 | 0.000 | 0.000 | 0.000 | 0.000 | 55.478 | 0.000 |
| P19 + CincaTE + CnGPAT9 + AtWRI | 0.000 | 32.975 | 0.000 | 0.000 | 29.893 | 0.000 |
| P19 + CuplaTE + CnGPAT9 + AtWRI | 0.000 | 9.743 | 0.000 | 0.000 | 55.084 | 0.000 |
| P19 + UmbcaTE + CnGPAT9 + AtWRI | 29.807 | 9.939 | 0.000 | 0.000 | 15.215 | 0.000 |
| P19 + CocnuTE2 + CnGPAT9 + AtWRI | 0.000 | 20.098 | 0.000 | 0.000 | 48.646 | 0.000 |
| P19 + CincaTE + CnGPAT9 + AtWRI + CnLPAAT | 4.924 | 38.894 | 0.000 | 0.000 | 22.078 | 0.000 |
| P19 + CuplaTE + CnGPAT9 + AtWRI + CnLPAAT | 0.000 | 9.483 | 0.000 | 0.000 | 57.458 | 0.000 |
| P19 + UmbcaTE + CnGPAT9 + AtWRI + CnLPAAT | 46.258 | 8.809 | 0.000 | 0.000 | 9.487 | 0.000 |
| P19 + CocnuTE2 + CnGPAT9 + AtWRI + CnLPAAT | 0.000 | 18.294 | 0.000 | 0.000 | 56.968 | 0.000 |
| P19 + CincaTE + CnGPAT9 + AtWRI + CnLPAAT + EgDGAT | 3.909 | 34.512 | 0.000 | 0.000 | 36.091 | 0.000 |
| P19 + CuplaTE + CnGPAT9 + AtWRI + CnLPAAT + EgDGAT | 0.000 | 4.605 | 0.000 | 0.000 | 56.818 | 0.000 |
| P19 + UmbcaTE + CnGPAT9 + AtWRI + CnLPAAT + EgDGAT | 51.506 | 7.067 | 0.000 | 0.000 | 11.083 | 0.000 |
| P19 + CocnuTE2 + CnGPAT9 + AtWRI + CnLPAAT + EgDGAT | 0.000 | 10.744 | 0.000 | 0.000 | 55.660 | 0.000 |
| P19 + CincaTE + AtGPAT9 + AtWRI + CnLPAAT + EgDGAT | 3.697 | 26.670 | 0.000 | 0.000 | 37.159 | 0.000 |
| P19 + CuplaTE + AtGPAT9 + AtWRI + CnLPAAT + EgDGAT | 1.737 | 4.336 | 0.000 | 0.000 | 54.136 | 0.000 |
| P19 + UmbcaTE + AtGPAT9 + AtWRI + CnLPAAT + EgDGAT | 49.371 | 6.898 | 0.000 | 0.000 | 10.168 | 0.000 |

TABLE 15
Total leaf fatty acid composition of TAG (% total TAG) of C17:0, C17:1, C18:0, C18:1, C19:0, C18:2, C18:3,
C20:0, C20:1, C22:0 and C24:0 fatty acids in Nicotiana benthamiana leaves infiltrated with various constructs.

| | | | | | | | | | | | |
|---|---|---|---|---|---|---|---|---|---|---|---|
| Genotype | C17:0 | C17:1 | C18:0 | C18:1 | C19:0 | C18:2 | C18:3 | C20:0 | C20:1 | C22:0 | C24:0 |

REPLICATE 1

| | | | | | | | | | | | |
|---|---|---|---|---|---|---|---|---|---|---|---|
| P19 | 0.000 | 0.000 | 7.003 | 3.505 | 0.000 | 7.516 | 13.827 | 1.204 | 1.260 | 1.303 | 2.428 |
| P19 + CincaTE + CnGPAT9 + AtWRI | 0.000 | 0.381 | 2.160 | 1.542 | 0.363 | 6.795 | 21.636 | 0.382 | 0.000 | 0.208 | 0.163 |
| P19 + CuplaTE + CnGPAT9 + AtWRI | 0.000 | 0.768 | 3.491 | 1.051 | 0.000 | 7.046 | 20.841 | 0.607 | 0.000 | 0.000 | 0.421 |
| P19 + UmbcaTE + CnGPAT9 + | 0.000 | 1.251 | 2.658 | 1.439 | 0.000 | 9.993 | 25.690 | 0.000 | 0.000 | 0.000 | 0.000 |
| AtWRI | | | | | | | | | | | |
| P19 + CocnuTE2 + CnGPAT9 + | 0.000 | 0.485 | 3.864 | 1.427 | 0.000 | 5.547 | 13.027 | 0.503 | 0.000 | 0.000 | 0.000 |
| AtWRI | | | | | | | | | | | |
| P19 + CincaTE + CnGPAT9 + AtWRI + | 0.000 | 0.296 | 1.824 | 2.156 | 0.000 | 4.653 | 17.211 | 0.307 | 0.000 | 0.000 | 0.000 |
| CnLPAAT | | | | | | | | | | | |
| P19 + CuplaTE + CnGPAT9 + AtWRI + | 0.000 | 0.242 | 2.812 | 5.820 | 0.616 | 6.643 | 14.892 | 0.515 | 0.000 | 0.203 | 0.000 |
| CnLPAAT | | | | | | | | | | | |
| P19 + UmbcaTE + CnGPAT9 + | 0.000 | 0.191 | 1.359 | 3.790 | 0.514 | 8.779 | 19.329 | 0.204 | 0.000 | 0.140 | 0.000 |
| AtWRI + CnLPAAT | | | | | | | | | | | |
| P19 + CocnuTE2 + CnGPAT9 + | 0.000 | 0.333 | 3.431 | 2.297 | 0.000 | 4.517 | 16.271 | 0.552 | 0.000 | 0.179 | 0.000 |
| AtWRI + CnLPAAT | | | | | | | | | | | |
| P19 + CincaTE + CnGPAT9 + AtWRI + | 0.000 | 0.244 | 2.274 | 6.967 | 0.414 | 7.193 | 8.577 | 0.560 | 0.000 | 0.297 | 0.137 |
| CnLPAAT + EgDGAT | | | | | | | | | | | |
| P19 + CuplaTE + CnGPAT9 + AtWRI + | 0.000 | 0.283 | 3.698 | 7.203 | 0.473 | 9.780 | 13.198 | 0.895 | 0.000 | 0.535 | 0.272 |
| CnLPAAT + EgDGAT | | | | | | | | | | | |
| P19 + UmbcaTE + CnGPAT9 + | 0.000 | 0.237 | 1.704 | 6.638 | 0.460 | 8.261 | 8.641 | 0.521 | 0.117 | 0.470 | 0.286 |
| AtWRI + CnLPAAT + EgDGAT | | | | | | | | | | | |
| P19 + CocnuTE2 + CnGPAT9 + | 0.000 | 0.457 | 3.117 | 1.071 | 0.324 | 4.844 | 20.248 | 0.459 | 0.000 | 0.205 | 0.000 |
| AtWRI + CnLPAAT + EgDGAT | | | | | | | | | | | |
| P19 + CincaTE + AtGPAT9 + AtWRI + | 0.000 | 0.299 | 2.232 | 4.203 | 0.290 | 5.963 | 12.828 | 0.500 | 0.000 | 0.233 | 0.089 |
| CnLPAAT + EgDGAT | | | | | | | | | | | |
| P19 + CuplaTE + AtGPAT9 + AtWRI + | 0.000 | 0.321 | 4.172 | 5.766 | 0.479 | 8.508 | 15.845 | 0.902 | 0.000 | 0.501 | 0.224 |
| CnLPAAT + EgDGAT | | | | | | | | | | | |
| P19 + UmbcaTE + AtGPAT9 + | 0.000 | 0.185 | 1.873 | 6.977 | 0.608 | 9.240 | 9.823 | 0.532 | 0.095 | 0.425 | 0.230 |
| AtWRI + CnLPAAT + EgDGAT | | | | | | | | | | | |

REPLICATE 2

| | | | | | | | | | | | |
|---|---|---|---|---|---|---|---|---|---|---|---|
| P19 | 0.000 | 0.000 | 5.724 | 0.000 | 0.000 | 9.527 | 21.105 | 0.000 | 0.000 | 0.000 | 0.000 |
| P19 + CincaTE + CnGPAT9 + AtWRI | 0.000 | 0.000 | 0.000 | 0.000 | 0.000 | 0.000 | 0.000 | 0.000 | 0.000 | 0.000 | 0.000 |
| P19 + CuplaTE + CnGPAT9 + AtWRI | 0.000 | 0.450 | 4.115 | 0.919 | 0.000 | 7.307 | 18.646 | 0.635 | 0.000 | 0.440 | 0.285 |
| P19 + UmbcaTE + CnGPAT9 + | 0.000 | 0.432 | 3.015 | 2.565 | 0.000 | 10.355 | 23.801 | 0.580 | 0.000 | 0.370 | 0.000 |
| AtWRI | | | | | | | | | | | |
| P19 + CocnuTE2 + CnGPAT9 + | 0.000 | 0.324 | 3.210 | 1.170 | 0.467 | 5.649 | 20.593 | 0.447 | 0.000 | 0.163 | 0.000 |
| AtWRI | | | | | | | | | | | |
| P19 + CincaTE + CnGPAT9 + AtWRI + | 0.000 | 0.206 | 2.042 | 3.281 | 0.299 | 4.527 | 15.495 | 0.334 | 0.000 | 0.122 | 0.000 |
| CnLPAAT | | | | | | | | | | | |
| P19 + CuplaTE + CnGPAT9 + AtWRI + | 0.000 | 0.342 | 3.649 | 1.568 | 0.000 | 6.412 | 20.615 | 0.553 | 0.000 | 0.298 | 0.000 |
| CnLPAAT | | | | | | | | | | | |
| P19 + UmbcaTE + CnGPAT9 + | 0.000 | 0.197 | 1.653 | 3.620 | 0.431 | 8.552 | 20.205 | 0.201 | 0.000 | 0.145 | 0.000 |
| AtWRI + CnLPAAT | | | | | | | | | | | |
| P19 + CocnuTE2 + CnGPAT9 + | 0.000 | 0.000 | 0.000 | 0.000 | 0.000 | 0.000 | 0.000 | 0.000 | 0.000 | 0.000 | 0.000 |
| AtWRI + CnLPAAT | | | | | | | | | | | |
| P19 + CincaTE + CnGPAT9 + AtWRI + | 0.000 | 1.023 | 2.522 | 4.695 | 0.000 | 7.688 | 11.161 | 0.000 | 0.000 | 0.000 | 0.000 |
| CnLPAAT + EgDGAT | | | | | | | | | | | |
| P19 + CuplaTE + CnGPAT9 + AtWRI + | 0.000 | 0.000 | 3.790 | 5.402 | 0.000 | 11.197 | 17.104 | 0.939 | 0.000 | 0.000 | 0.000 |
| CnLPAAT + EgDGAT | | | | | | | | | | | |
| P19 + UmbcaTE + CnGPAT9 + | 0.000 | 0.000 | 1.950 | 6.434 | 0.000 | 10.572 | 10.877 | 0.615 | 0.000 | 0.598 | 0.000 |
| AtWRI + CnLPAAT + EgDGAT | | | | | | | | | | | |
| P19 + CocnuTE2 + CnGPAT9 + | 0.000 | 2.364 | 2.998 | 1.381 | 0.000 | 6.570 | 21.589 | 0.000 | 0.000 | 0.000 | 0.000 |
| AtWRI + CnLPAAT + EgDGAT | | | | | | | | | | | |
| P19 + CincaTE + AtGPAT9 + AtWRI + | 0.000 | 0.962 | 2.526 | 6.126 | 0.000 | 9.898 | 12.288 | 0.603 | 0.000 | 0.000 | 0.000 |
| CnLPAAT + EgDGAT | | | | | | | | | | | |
| P19 + CuplaTE + AtGPAT9 + AtWRI + | 0.000 | 1.141 | 3.660 | 6.542 | 0.000 | 11.577 | 14.693 | 0.794 | 0.000 | 0.424 | 0.000 |
| CnLPAAT + EgDGAT | | | | | | | | | | | |
| P19 + UmbcaTE + AtGPAT9 + | 0.000 | 0.000 | 1.909 | 6.636 | 0.000 | 11.998 | 10.677 | 0.000 | 0.000 | 0.000 | 0.000 |
| AtWRI + CnLPAAT + EgDGAT | | | | | | | | | | | |

REPLICATE 3

| | | | | | | | | | | | |
|---|---|---|---|---|---|---|---|---|---|---|---|
| P19 | 0.000 | 0.000 | 0.000 | 0.000 | 0.000 | 19.966 | 24.557 | 0.000 | 0.000 | 0.000 | 0.000 |
| P19 + CincaTE + CnGPAT9 + AtWRI | 0.000 | 1.600 | 2.296 | 2.966 | 0.000 | 10.225 | 20.046 | 0.000 | 0.000 | 0.000 | 0.000 |
| P19 + CuplaTE + CnGPAT9 + AtWRI | 0.000 | 0.000 | 3.044 | 2.041 | 0.000 | 10.711 | 19.376 | 0.000 | 0.000 | 0.000 | 0.000 |
| P19 + UmbcaTE + CnGPAT9 + | 0.000 | 0.000 | 2.821 | 3.186 | 0.000 | 14.113 | 24.919 | 0.000 | 0.000 | 0.000 | 0.000 |
| AtWRI | | | | | | | | | | | |
| P19 + CocnuTE2 + CnGPAT9 + | 0.000 | 1.637 | 2.992 | 1.264 | 0.000 | 6.611 | 18.753 | 0.000 | 0.000 | 0.000 | 0.000 |
| AtWRI | | | | | | | | | | | |
| P19 + CincaTE + CnGPAT9 + AtWRI + | 0.000 | 2.465 | 1.645 | 1.269 | 0.000 | 6.427 | 22.298 | 0.000 | 0.000 | 0.000 | 0.000 |
| CnLPAAT | | | | | | | | | | | |
| P19 + CuplaTE + CnGPAT9 + AtWRI + | 0.000 | 0.000 | 2.864 | 3.499 | 0.000 | 7.993 | 18.702 | 0.000 | 0.000 | 0.000 | 0.000 |
| CnLPAAT | | | | | | | | | | | |
| P19 + UmbcaTE + CnGPAT9 + | 0.000 | 0.000 | 1.356 | 4.320 | 0.000 | 10.699 | 19.070 | 0.000 | 0.000 | 0.000 | 0.000 |
| AtWRI + CnLPAAT | | | | | | | | | | | |
| P19 + CocnuTE2 + CnGPAT9 + | 0.000 | 0.000 | 3.467 | 0.000 | 0.000 | 6.091 | 15.180 | 0.000 | 0.000 | 0.000 | 0.000 |
| AtWRI + CnLPAAT | | | | | | | | | | | |
| P19 + CincaTE + CnGPAT9 + AtWRI + | 0.000 | 0.875 | 2.053 | 5.925 | 0.000 | 7.047 | 9.165 | 0.422 | 0.000 | 0.000 | 0.000 |
| CnLPAAT + EgDGAT | | | | | | | | | | | |
| P19 + CuplaTE + CnGPAT9 + AtWRI + | 0.000 | 1.103 | 3.685 | 8.285 | 0.000 | 10.471 | 15.034 | 0.000 | 0.000 | 0.000 | 0.000 |
| CnLPAAT + EgDGAT | | | | | | | | | | | |
| P19 + UmbcaTE + CnGPAT9 + | 0.000 | 0.631 | 1.506 | 6.663 | 0.000 | 11.367 | 9.290 | 0.462 | 0.000 | 0.425 | 0.000 |
| AtWRI + CnLPAAT + EgDGAT | | | | | | | | | | | |
| P19 + CocnuTE2 + CnGPAT9 + | 0.000 | 1.705 | 3.665 | 2.985 | 0.000 | 7.058 | 18.182 | 0.000 | 0.000 | 0.000 | 0.000 |
| AtWRI + CnLPAAT + EgDGAT | | | | | | | | | | | |
| P19 + CincaTE + AtGPAT9 + AtWRI + | 0.000 | 0.816 | 2.447 | 7.987 | 0.000 | 9.927 | 10.359 | 0.598 | 0.000 | 0.339 | 0.000 |
| CnLPAAT + EgDGAT | | | | | | | | | | | |
| P19 + CuplaTE + AtGPAT9 + AtWRI + | 0.000 | 1.020 | 3.588 | 7.767 | 0.000 | 11.969 | 14.237 | 0.765 | 0.000 | 0.445 | 0.000 |
| CnLPAAT + EgDGAT | | | | | | | | | | | |
| P19 + UmbcaTE + AtGPAT9 + | 0.000 | 0.647 | 1.538 | 8.125 | 0.000 | 12.136 | 10.070 | 0.433 | 0.000 | 0.377 | 0.236 |
| AtWRI + CnLPAAT + EgDGAT | | | | | | | | | | | |

### Discussion

In the seeds of native plants, the incorporation of unusual fatty acids is almost exclusively confined to TAG and typically excluded from membrane lipids, most likely because they interfere with proper membrane functions and are often deleterious to the plant cells (Millar et al., 2000). A different scenario has been observed in transgenic plants that have attempted to modify the oil fatty acid profiles, such as increasing the lauric acid content (Knutzon et al., 1999). Although high levels of laurate accumulation in plant oils have been achieved in the seeds of transgenic canola, there was a significant level of laurate being sequestered in PC during seed development (Wiberg et al., 1997). In that work, de novo DAG containing laurate was not efficiently converted to TAG by the resident DGAT but was instead converted to the membrane lipid PC. The native canola LPCAT lacked the capability to handle MCFAs (Zhang et al., 2015) so the route to PC could be through PDCT or CPT activities. Consequently, this inefficient utilization of laurate for TAG synthesis was also associated with a negatively correlated penalty in total oil yields (Knutzon et al., 1999).

Similar to the expression of MCFA in seed oil, the over expression of MCFA in the leaf cells described here with the co-expression of CnGPAT9 and CnLPAAT identified a metabolic bottleneck through the sequestering of MCFA in PC. The low abundance of MCFA-containing DAG species suggested that de novo DAG containing MCFA was quickly converted to PC through the activities of PDCT or CPT or both, due to the absence of a DGAT capable of using the MCFA-containing DAG for TAG assembly. The inventors showed that the addition to the enzyme combination of a DGAT with a preference for MCFA as substrate, relative to one or more C18 substrates such as oleic acid, LA or ALA, promoted synthesis of MCFA-enriched TAG and relieved this bottleneck. Endogenous PDAT may also be involved in the maintenance of membrane homeostasis, through the removal of unusual fatty acids from the membrane lipids and sequestering them into TAG (Fan et al., 2014; Fan et al., 2013a and b). This study demonstrated that the expression of the DGAT from a species such as E. guineensis (EgDGAT1) was sufficient to restore membrane homeostasis by reducing the accumulation of MCFA in PC. The expression of EgDGAT1 proved that a DGAT with MCFA substrate preference was beneficial for the efficient assembly of TAG and increased TAG content in the plant cells. The reconfigured Kennedy pathway for improving MCFA incorporation into TAG is expected to benefit seedoil composition and TAG content as well.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

The references listed below are incorporated by reference where cited in the Specification:
Adhikari et al. (2016) Plant Physiol 171:179-191.
Alemanno et al. (2008) Planta 227:853-866.
Almeida and Allshire (2005) TRENDS Cell Biol. 15:251-258.
Alonso et al. (2009) Plant Cell 21: 1747-1761.
Alonso et al. (2010) Green Chem. 12:1493-1513.
Alvarez et al. (2000) Theor. Appl. Genet. 100:319-327.
Andre at al (2012) Proc. Natl. Acad. Sci. U.S.A. 109:10107-10112.
Arkcoll (1988) Lauric Oil Resources. Economic Botany 42:195-205.
Bartlett et al. (2008) Plant Methods 4:22.
Basiron and Weng (2004) Journal of Oil Palm Research 16.
Bates et al. (2014) PNAS USA 111:1204-1209.
Bates and Browse (2011) Plant J 68:387-399.
Bates and Browse (2012) Frontiers in Plant Science 3:147.
Baud et al. (2007) Plant J. 50:825-838.
Baud and Lepiniec (2010) Progr. Lipid Res. 49: 235-249.
Baumlein et al. (1991) Mol. Gen. Genet. 225:459-467.
Baumlein et al. (1992) Plant J. 2:233-239.
Belide et al. (2013) Plant Cell Tiss. Org. Cult. DOI 10.1007/s11240-013-0295-1.
Ben Saad et al. (2011) Transgenic Res 20: 1003-1018.
Bibikova et al. (2002) Genetics 161:1169-1175.
Bihmidine et al. (2015) BMC Plant Biology 15:186.
Bihmidine et al. (2016) Plant Signaling & Behaviour 11: e1117721.
Bligh and Dyer (1959) Canadian Journal of Biochemistry and Physiology 37:911-917.
Boutilier et al. (2002) Plant Cell 14:1737-1749.
Bouvier-Nave et al. (2000) European Journal of Biochemistry/FEBS 267:85-96.
Bradford (1976) Anal. Biochem. 72:248-254.
Broothaerts et al. (2005) Nature 433:629-633.
Broun et al. (1998) Plant J. 13:201-210.
Browse et al. (1986) Biochem J 235: 25-31.
Buchanan-Wollaston (1994) Plant Physiol. 105:839-846.
Burgal et al. (2008) Plant Biotechnol J 6:819-831.
Busk et al. (1997) Plant J. 11:1285-1295.
Cai et al. (2015) Plant Cell 27:2616-2636.
Cao (2011) BMC Research Notes 4:249.
Cao et al. (2007) J. Lipid Res. 48:583-591.
Capuano et al. (2007) Biotechnol. Adv. 25:203-206.
Chapman and Ohlogge (2012) J. Biol. Chem. 287:2288-2294.
Chen et al (2011) Plant Physiol. 155:851-865.
Chen et al. (2016) International Journal of Molecular Sciences 17:507.
Chikwamba et al. (2003) Proc. Natl. Acad. Sci. U.S.A. 100:11127-11132.
Christensen et al. (1992) Plant Mol Biol 18:675-689.
Christie (1993) Advances in Lipid Methodology-Two, Oily Press, Dundee, pp 195-213.
Chung et al. (2006) BMC Genomics 7:120.
Comai et al. (2004) Plant J 37: 778-786.
Cong et al. (2013) Science 339:819-823.
Corrado and Karali (2009) Biotechnol. Adv. 27:733-743.
Coutu et al. (2007) Transgenic Res. 16:771-781.
Dahlqvist et al. (2000), Proc. Natl. Acad. Sci. U.S.A. 97: 6487-6492.
Damaj et al., (2010) Planta 231:1439-1458.
Dandik and Aksoy (1998) Fuel Process Technol. 57: 81-92.
Dauk et al (2007) Plant Sci. 173:43-49.
Deruyffelaere et al. (2015) Plant Cell Physiol 56:1374-1387.
Dehesh (2001) European Journal of Lipid Science and Technology 103:688-697.
Durrett et al. (2008) Plant J. 54:593-607.
Dussert et al. (2013) Plant Physiol 162:1337-1358.
Dyer et al. (2002) Plant Physiol. 130:2027-2038.
Eastmond et al. (2006) Plant Cell 18: 665-675.
Eccleston et al. (1996) Planta 198:46-53.
Eccleston and Ohlrogge (1998) The Plant Cell Online 10:613-621.
Edem (2002) Plant Foods for Human Nutrition 57:319-341.
Ellerstrom et al. (1996) Plant Mol. Biol. 32:1019-1027.
El Tahchy et al. (2017) FEBS Letters 591:448-456.
Endalew et al. (2011) Biomass and Bioenergy 35:3787-3809.
Fan et al. (2013a) Plant Cell 25: 3506-3518.
Fan et al. (2013b) Plant Journal 76: 930-942.
Fan et al. (2014) Plant Cell 26: 4119-4134.
Fan et al. (2015) Plant Cell 27: 2941-2955.
FAO Animal Production and Health Proceedings (2002) Protein sources for the animal feed industry, Expert Consultation and Workshop, Bangkok.
Feeney et al. (2012) Plant Physiol 162: 1881-1896.
Finkelstein et al. (1998) Plant Cell 10:1043-1054.
Froissard et al. (2009) FEMS Yeast Res 9:428-438.
Gan (1995) Molecular characterization and genetic manipulation of plant senescence. PhD thesis. University of Wisconsin, Madison.
Gan and Amasino (1995) Science 270:1986-1988.
Gazzarrini et al. (2004) Dev. Cell 7:373-385.
Geurin et al. (2016) Plant Biotech. J 87: 423-441.
Ghosal et al. (2007) Biochimica et Biophysica Acta 1771:1457-1463.
Ghosh et al. (2009) Plant Physiol. 151:869-881.
Gidda et al (2013) Plant Signaling Behav. 8:e27141.
Girijashankar and Swathisree, (2009) Physiol. Mol. Biol. Plants 15: 287-302.
Gong and Jiang (2011) Biotechnol. Lett. 33:1269-1284.
Gould et al. (1991) Plant Physiol. 95:426-434.
Greenwell et al. (2010) J. R. Soc. Interface 7:703-726.
Guan et al. (2015) Lipids 50:407-416.
Gurel et al. (2009) Plant Cell Rep. 28:429-444.
Gutierrew et al. (2013) BMC Biotechnol. 13:40.
Hedrich et al. (2015) Curr Opin Plant Biol 25: 63-70.
Henikoff et al. (2004) Plant Physiol. 135:630-636.
Hershey and Stoner (1991) Plant Mol. Biol. 17:679-690.
Hinchee et al. (1988) Biotechnology 6:915-922.
Hom et al. (2007) Euphytica 153:27-34.
Horn et al. (2013). Plant Physiol 162:1926-1936.
Horvath et al. (2000) Proc. Natl. Acad. Sci. U.S.A. 97:1914-1919.
Hsiao and Tzen (2011) Plant Physiol. Biochem. 49: 77-81.
Hsieh and Huang (2004) Plant Physiol 136:3427-3434.
Huang (1996) Plant Physiol. 110:1055-1061.
Huang and Huang (2016) Plant Physiol. 171: 1867-1878.
Ichihara et al (1988) Biochim. Biophys. Acta 958:125-129.
Ikeda et al. (2006) Pl Biotech J. 23: 153-161.
Iwabuchi et al. (2003) J. Biol. Chem. 278:4603-4610.
James et al. (2010) Proc. Natl. Acad. Sci. USA 107:17833-17838.
Jepson et al. (1994) Plant Mol. Biol. 26:1855-1866.
Jing et al. (2011) BMC Biochemistry 12:44.
Jolivet et al. (2014) Plant Physiol. Biochem. 42:501-509.
Jones et al. (1995) Plant Cell 7: 359-371.
Karmakar et al. (2010) Bioresource Technology 101:7201-7210.
Kelly et al. (2011) Plant Physiol. 157: 866-875.
Kelly et al (2013a) Plant Biotech. J. 11:355-361.
Kelly et al. (2013b) Plant Physiol. 162:1282-1289.
Kereszt et al. (2007) Nature Protocols 2:948-952.
Knutzon et al. (1995) Plant Physiol 109:999-1006.
Knutzon et al. (1999) Plant Physiology 120:739-746.
Kim et al. (2014) Biotechnology for Biofuels 7:36.
Kim et al. (2015a) Plant J 84:1021-1033.
Kim et al. (2015b) Journal of Experimental Botany 66:4251-4265.
Kim et al. (1996) Proc. Natl. Acad. Sci. USA 93:1156-1160.
Kim et al. (2016) Plant Physiol 171: 1951-1964.
Klemens et al. (2013) Plant Physiol 163: 1338-1352.
Koziel et al. (1996) Plant Mol. Biol. 32:393-405.
Kuhn et al. (2009) J. Biol. Chem. 284:34092-102.
Kunst et al. (1988) Proc. Natl. Acad. Sci. U.S.A. 85:4143-4147.
Kwong et al. (2003) Plant Cell 15:5-18.
Lacroix et al. (2008) Proc. Natl. Acad. Sci. U.S.A. 105: 15429-15434.
Laibach et al. (2015). J. Biotechnol. 201: 15-27.
Lardizabal et al. (2008) Plant Physiol. 148: 89-96.
Laureles et al. (2002) J Agric Food Chem 50:1581-1586.
Lebrun et al. (1987) Nucl. Acids Res. 15:4360.
Laux et al. (1996) Development 122: 87-96.
Lazo et al. (1991) Bio/Technology 9:963-967.
Lee et al. (1998) Science 280:915-918.
Lee et al. (2003) Proc. Natl. Acad. Sci. U.S.A. 100:2152-2158.
Li et al. (1996) FEBS Lett. 379:117-121.
Li et al. (2006) Phytochemistry 67: 904-915.
Li et al. (2017) Plant Physiol 173:2208-2224.
Lin et al. (2005) Plant Physiol. Biochem. 43:770-776.
Linder et al. (2005). FEMS Microbiol. Rev. 29: 877-896.
Liu and Godwin (2012). Plant Cell Reports 31, 999-1007.
Liu et al. (2010) Plant Physiol. Biochem. 48: 9-15.
Liu et al. (2012) Prog Lipid Res 51:350-377.
Liu et al. (2012) J Exp Bot 63: 3727-3740.
Liu et al. (2014) BMC Plant Biol. 14:73.
Lotan et al. (1998) Cell 93: 1195-1205.
Lu et al. (2009) Proc Natl Acad of Sci USA 106:18837-18842.
Luerssen et al. (1998) Plant J. 15: 755-764.
Lui et al. (2009) J. Agric. Food Chem. 57: 2308-2313.
Ma et al. (2016) Plant Journal doi: 10.1111/tpj.13244.
MacEachran et al. (2010). Appl. Environ. Microbiol. 76: 7217-7225.
Maher and Bressler (2007) Bioresource Technology 98:2351-2368.
Matsuoka et al. (1994) Plant J. 6:311-319.
Matsuoka and Minami (1989) Eur. J. Biochem. 181: 593-598.
McCleary et al. (2013) J AOAC Int 93:221-233.
McCleary et al. (2015) Starch 67:860-883.
McElroy et al. (1990) Plant Cell 2: 163-171.
Mckinley et al. (2016) Plant Journal: doi: 10.1111/tpj. 13269.
Meier et al. (1997) FEBS Lett. 415:91-95.
Millar et al. (2000) Trends in Plant sScience 5:95-101.
Millar and Waterhouse (2005). Funct Integr Genomics 5:129-135.
Miller (1984). Crop Sci 24:1224-1224.
Mojica et al. (2000) Mol Microbiol 36:244-246.
Moreno-Perez (2012) PNAS 109:10107-10112.
Mongrand et al. (1998) Phytochemistry 49:1049-1064.
Moyle and Birch (2013) Theor. Appl. Genet. 126:1775-1782.
Mu et al. (2008) Plant Physiol. 148:1042-1054.
Mudge et al. (2013) Plant Biotechnol. J. 11:502-509.
Murashige and Skoog (1962). Physiol Plant 15:473-497.
Murphy et al. (2012). Protoplasma 249:541-585.
Needleman and Wunsch (1970) J. Mol Biol. 45: 443-453.
Nilsson et al. (2012) Physiol. Plantarum 144: 35-47.
Nomura et al. (2000) Plant Mol. Biol. 44: 99-106.
OECD/FAO (2015) OECD-FAO Agricultural Outlook (Edition 2015). Paris:OECD Publishing.
Ohlrogge and Browse (1995) Plant Cell 7: 957-970.
Padidam (2003) Curr. Opin. Plant Biol. 6:169-77.
Padidam et al. (2003) Transgenic Res. 12:101-9.
Parthibane et al. (2012a) J. Biol. Chem. 287:1946-1965.
Parthibane et al. (2012b) Plant Physiol. 159:95-104.
Pasquinelli et al. (2005). Curr. Opin. Genet. Develop. 15:200-205.
Perez-Vich et al. (1998) J.A.O.C.S. 75:547-555.
Perrin et al. (2000) Mol. Breed. 6:345-352.
Petrie et al. (2012) PLOS One 7: e35214.
Phillips et al. (2002) Journal of Food Composition and Analysis 12:123-142.
Potenza et al. (2004) In Vitro Cell Dev. Biol. Plant 40:1-22.
Poxleitner et al. (2006) Plant J. 47:917-933.
Prosky et al. (1985) J AOAC Chem 68:677-679.
Pyc et al. (2017) Trends in Plant Sci. 22:596-609.
Qazi et al. (2012) Journal of Plant Physiology 169: 605-613.
Qiu et al. (2001) J. Biol. Chem. 276:31561-3156.
Reynolds et al. (2015) Frontiers in Plant Science 6.
Robson et al. (2004) Plant Biotechnol J 2:101-112.
Rossell and Pritchard (1991) Analysis of Oilseeds, Fats and Fatty Foods. Elsevier
Ruuska et al. (2002) Plant Cell 14:1191-1206.
Saha et al. (2006) Plant Physiol. 141:1533-1543.
Sanjaya et al. (2011) Plant Biotechnol J 9:874-883.
Santos-Mendoza et al. (2005) FEBS Lett. 579:4666-4670.
Santos-Mendoza et al. (2008) Plant J. 54:608-620.
Schneider et al. (2012) Plant Biol 14: 325-336.
Schnurr et al. (2002) Plant Physiol 129:1700-1709.
Shaw et al. (1959) J Soil Sci 10:316-326.
Shen et al. (2010) Plant Phys. 153: 980-987.
Shen et al. (2014). Biochem. Biophys. Res. Comm. 448: 365-371.
Semwal et al. (2011) Bioresource Technology 102:2151-2161.
Shen et al. (2010) Plant Physiol. 153:980-987.
Shiina et al. (1997) Plant Physiol. 115:477-483.
Shimada and Hara-Nishimura (2010) Biol. Pharm. Bull. 33:360-363.
Shimada et al. (2014) Plant Physiol. 164:105-118.
Shockey et al. (2002) Plant Physiol 129:1710-1722.
Shockey et al. (2016) Plant Physiol 170:163-179.
Singer et al. (2016) Journal of Experimental Botany 67:4627-4638.
Slade and Knauf (2005) Transgenic Res. 14: 109-115.
Smith et al. (2000) Nature 407:319-320.
Somerville et al. (2000) Lipids. In BB Buchanan, W Gruissem, R L Jones, eds, Biochemisty and
Molecular Biology of Plants. American Society of Plant Physiologists, Rockville, MD, pp 456-527.
Sorokin et al. (2009) Biochemistry Biokhimiia 74:1411-1442.
Srinivasan et al. (2007) Planta 225:341-51.
Stalker et al. 1988 Science 242: 419-423.
Stone et al. (2001) Proc. Natl. Acad. Sci. U.S.A. 98: 11806-11811.
Stone et al. (2008) Proc. Natl. Acad. Sci. U.S.A. 105: 3151-3156.
Tai et al. (2002). Biosci. Biotechnol. Biochem. 66: 2146-2153.
Tan et al. (2011) Plant Physiol. 156:1577-1588.
Taylor (1997) The Plant Cell 9:1245-1249.
Thillet et al. (1988) J. Biol. Chem 263:12500-12508.
Tingay et al. (1997) Plant J. 11:1369-1376.
Tjellstrom et al. (2013) FEBS Lett 587:936-942.
To et al. (2012) Plant Cell 24:5007-5023.
Ulmasov et al. (1995) Plant Physiol. 108:919-927.
van de Loo et al. (1995) Proc Natl Acad Sci USA. 92:6743-6747.
van Erp et al. (2011) Plant Physiol 155:683-693.
van Erp et al. (2015) Plant Physiol 168:36-46.
Vanhercke et al. (2013) FEBS Letters 587:364-369.
Vanhercke et al. (2014a). Plant Biotech. J. 12:231-239.
Vanhercke et al. (2014b) Biocatalysis and Agricultural Biotechnology 3:75-80.
Vanhercke et al. (2017) Metabolic Engineering 39:237-246.
Vieler et al. (2012) Plant Physiol. 158:1562-1569.
Voinnet et al. (2003) Plant J. 33:949-956.
Voelker et al. (1992) Science 257:72-74.
Voelker et al. (1996) Plant J 9:229-241.
Wang et al. (2002) Plant J 32:831-843.
Wang et al. (2007) Plant J 52: 716-729.
Waterhouse et al. (1998). Proc. Natl. Acad. Sci. U.S.A. 95:13959-13964.
Weissbach and Weissbach, (1989) Methods for Plant Mol Biol, Academic Press.
Weissbach et al., In: Methods for Plant Molecular Biology, Academic Press, San Diego, Calif., (1988).
Weissman (2001) Molec Cell Biol. 2:169-178.
Wesley et al. (2001) Plant J. 27:581-590.
Wiberg et al. (1997) Planta 203:341-348.
Wiberg et al. (2000) Planta 212:33-40.
Winichayakul et al. (2013) Plant Physiol. 162:626-639.
Wood (2014) EMBO Reports 15:201-202.
Wood et al. (2009) Plant Biotech. J. 7: 914-924.
Wu et al. (2014) In Vitro Cellular and Dev. Biol.-Plant 50:9-18.
Xu et al. (2008) Plant Biotechnol J 6:799-818.
Xu et al (2010) Plant and Cell Physiol. 51:1019-1028.
Xu et al (2005) Plant Cell 17:3094-3110.
Xu et al (2008) Plant Cell 20:2190-2204
Yamagishi et al. (2005) Pl Physiol 139: 163-173.
Yamasaki et al. (2004) Plant Cell 16:3448-3459.
Yang et al. (2003) Planta 216:597-603.
Yang et al. (2010) Proc. Natl. Acad. Sci. U.S.A.107:12040-12045.
Yeap et al. (2017) Plant Journal 91: 97-113.
Yen et al. (2002) Proc. Natl. Acad. Sci. U.S.A. 99:8512-8517.
Yen et al. (2005) J. Lipid Res. 46: 1502-1511.
Yokoyama et al. (1994) Mol Gen Genet 244: 15-22.
Yuan et al. (1995) Proc. Natl. Acad. Sci. U.S.A. 92: 10639-10643.
Zale et al. (2016) Plant Biotech J. 14: 661-669.
Zhang et al. (2015) PLOS ONE 10, e0144653.
Zheng et al. (2009) Pl Physiol 21: 2563-2577.
Zhou et al. (2011) J Biol Chem 286:43644-43650.
Zolman et al. (2001) Plant Physiol. 127:1266-1274.

1-52. (canceled)

53. A process for producing extracted plant lipid, comprising the steps of:
a) obtaining one or more vegetative plant parts comprising lipid, the lipid comprising a total fatty acid content which comprises fatty acids in an esterified form, the fatty acids comprising a level of total medium chain fatty acids (MCFA) that is at least 25% of the total fatty acid content on a weight basis, wherein the MCFA comprises a level of myristic acid (C14:0), wherein the level of myristic acid is between 20% and 50% of the total fatty acid content on a weight basis, and/or between 20% and 50% of the fatty acid content esterified in the form of triacylglycerol (TAG) is myristic acid on a weight basis, and
b) extracting lipid from the vegetative plant parts,
thereby producing the extracted plant lipid.

54. The process of claim 53, wherein the extracted plant lipid is in the form of an oil, wherein between 95% and 98% by weight of the oil is the lipid.

55. The process of claim 53, wherein the vegetative plant part is a plant leaf or stem.

56. The process of claim 53 which further comprises recovering the extracted plant lipid by collecting it in a container and/or one or more of degumming, deodorising, decolourising, drying, fractionating the extracted plant lipid, removing wax esters from the extracted plant lipid, or analysing the fatty acid composition of the extracted plant lipid.

57. The process of claim 53, wherein the level of medium chain fatty acids in the total fatty acid content of the vegetative plant parts is between 25% and 55% of the total fatty acid content on a weight basis.

58. The process of claim 57, wherein the total fatty acid content of the extracted lipid, and/or the total fatty acid content of triacylglycerol of the extracted lipid comprises a level of palmitic acid (C16:0) of between 2% and 18%.

59. The process of claim 57, wherein the total fatty acid content of the extracted plant lipid and/or the total fatty acid content of the triacylglycerol of the extracted plant lipid comprises a level of lauric acid (C12:0) of between 1% and 10%.

60. The process of claim 57, wherein the total fatty acid content of the extracted plant lipid, and/or the total fatty acid content of triacylglycerol of the extracted plant lipid comprises:
i) a level of oleic acid of less than 10%;
ii) a level of linoleic acid (LA) of less than 20%; and
iii) a level of α-linolenic acid (ALA) of less than 20%.

61. The process of claim 57, wherein the extracted plant lipid comprises tri-myristin (tri-C14:0).

62. The process of claim 53, wherein the process further comprises converting the extracted plant lipid to a hydrocarbon product.

63. The process of claim 62, wherein the hydrocarbon product comprises fatty acid esters, an alkane, an alkene, or combinations thereof.

64. A process for producing an industrial product, the process comprising the steps of:
i) obtaining extracted plant lipid which was produced by the process of claim 53,
ii) converting at least some of the extracted plant lipid to the industrial product by applying heat, chemical, or enzymatic means, or any combination thereof, to the extracted plant lipid, and
iii) recovering the industrial product,
thereby producing the industrial product.

65. A process for producing fuel, the process comprising the steps of:
i) obtaining extracted plant lipid which was produced by the process of claim 53, and
ii) reacting the extracted plant lipid with an alcohol, optionally in the presence of a catalyst, to produce alkyl esters.

66. The process of claim 65, further comprising a step of blending the alkyl esters with petroleum based fuel.

67. A process for producing a synthetic diesel fuel, the process comprising:
i) obtaining extracted plant lipid which was produced by the process of claim 53, and
ii) converting the extracted plant lipid to a synthetic diesel fuel by a process comprising fractionation.

68. The process of claim 67, wherein step ii) comprises selecting hydrocarbon compounds which condense between 150° C. and 200° C. or between 200° C. and 300° C.

69. Extracted plant lipid which was extracted from a vegetative plant part, comprising a total fatty acid content which comprises fatty acids in an esterified form, the fatty acids comprising a level of medium chain fatty acids in the total fatty acid content of the extracted plant lipid that is at least 25% on a weight basis, wherein the medium chain fatty acids comprise a level of myristic acid (C14:0), wherein the level of myristic acid in the total fatty acid content of the extracted plant lipid is between 20% and 50% on a weight basis, and/or between 20% and 50% of the fatty acid content esterified in the form of triacylglycerol (TAG) of the extracted plant lipid is myristic acid on a weight basis.

70. The extracted plant lipid of claim 69, wherein the level of medium chain fatty acids in the total fatty acid content of the extracted plant lipid is between 25% and 55% of the total fatty acid content on a weight basis.

71. The extracted plant lipid of claim 70, wherein the total fatty acid content of the extracted plant lipid, and/or the total fatty acid content of triacylglycerol of the extracted plant lipid comprises a level of palmitic acid (C16:0) of between 2% and 18%.

72. The extracted plant lipid of claim 70, wherein the total fatty acid content of the extracted plant lipid, and/or the total fatty acid content of triacylglycerol of the extracted plant lipid comprises a level of lauric acid (C12:0) of between 1% and 10%.

73. The extracted plant lipid of claim 70, wherein the total fatty acid content of the extracted plant lipid, and/or the total fatty acid content of triacylglycerol of the extracted plant lipid comprises
i) a level of oleic acid of less than 10%;
ii) a level of linoleic acid (LA) of less than 20%; and
iii) a level of α-linolenic acid (ALA) of less than 20%.

74. The extracted plant lipid of claim 70, wherein the extracted plant lipid comprises tri-myristin (tri-C14:0).


SEQUENCE LISTING (152 sequences)

SEQ ID NO: 1 - protein, 520 aa, Arabidopsis thaliana
MAILDSAGVTTVTENGGGEFVDLDRLRRRKSRSDSSNGLLLSGSDNNSPSDDVGAPADVRDRIDSVVNDDAQGTANLAGDNNGGGDNNGGGRGGGEGRGNADATFTYRPSVPAHRRARESPLSSDAIFKQSHAGLFNLCVVVLIAVNSRLIIENLMKYGWLIRTDFWFSSRSLRDWPLFMCCISLSIFPLAAFTVEKLVLQKYISEPVVIFLHIIITMTEVLYPVYVTLRCDSAFLSGVTLMLLTCIVWLKLVSYAHTSYDIRSLANAADKANPEVSYYVSLKSLAYFMVAPTLCYQPSYPRSACIRKGWVARQFAKLVIFTGFMGFIIEQYINPIVRNSKHPLKGDLLYAIERVLKLSVPNLYVWLCMFYCFFHLWLNILAELLCFGDREFYKDWWNAKSVGDYWRMWNMPVHKWMVRHIYFPCLRSKIPKTLAIIIAFLVSAVFHELCIAVPCRLFKLWAFLGIMFQVPLVFITNYLQERFGSTVGNMIFWFIFCIFGQPMCVLLYYHDLMNRKGSMS

SEQ ID NO: 2 - skipped

SEQ ID NO: 3 - protein, 4 aa, synthetic construct
  REGION 1..4: note = conserved sequence
HPHG

SEQ ID NO: 4 - protein, 4 aa, synthetic construct
  REGION 1..4: note = conserved sequence
EPHS

SEQ ID NO: 5 - protein, 24 aa, synthetic construct
  REGION 1..24: note = conserved sequence
  VARIANT 2: note = X can be any amino acid
  VARIANT 5: note = X can be any amino acid
  VARIANT 6: note = Lysine (K) or Arginine (R)
  VARIANT 7: note = X can be any amino acid
  VARIANT 9..11: note = X can be any amino acid
  VARIANT 13..15: note = X can be any amino acid
  VARIANT 16: note = Leucine (L) or Valine (V)
  VARIANT 19..21: note = X can be any amino acid
  VARIANT 24: note = Glutamic Acid (E) or Glutamine (Q)
RXGFXXXAXXXGXXXXVPXXXFGX

SEQ ID NO: 6 - protein, 8 aa, synthetic construct
  REGION 1..8: note = conserved sequence
  VARIANT 3: note = X can be any amino acid
  VARIANT 5..7: note = X can be any amino acid
FLXLXXXN

SEQ ID NO: 7 - protein, 15 aa, synthetic construct
  REGION 1..15: note = conserved sequence
GDLVICPEGTTCREP

SEQ ID NO: 8-9 - skipped (2 entries)

SEQ ID NO: 10 - protein, 393 aa, Sorghum bicolor
MASPNPEAAAGLQTVAVAAGGGEGGSSSSLGAVAGAAAVSSSGELVPRRSLAVRKERVCTAKERISRMPPCAAGKRSSIYRGVTRHRWTGRYEAHLWDKSTWNQNQNKKGKQGAYDDEEAAARAYDLAALKYWGAGTQINFPVSDYARDLEEMQMISKEDYLVSLRRQLHNSRWDTSLGLGNDYMSLSCGKDIMLDGKFAGSFGLERKIDLTNYIRWWLPKKTRQSDTSKTEEIADEIRAIESSMQQTEPYKLPSLGLGSPSKPSSVGLSACSILSQSDAFKSFLEKSTKLSEECTLSKEIVEGKTVASVPATGYDTGAININMNELLVQRSTYSMAPVMPTPMKTTWSPADPSVDPLFWSNFVLPSSQPVTMATITTTTNEVSSSDPFQSQE

SEQ ID NO: 11 - protein, 428 aa, Lupinus angustifolius
MASSSSDPGKSEIGGGAAETSEAAAVAVAVTNDQSLLYRGLKKAKKERGCTAKERISKMPPCAAGKRSSIYRGVTRHRWTGRYEAHLRDKSTWNQNQNKKGKQVYLGAYDDEEAAARAYDLAALKYWGPGTLINFPVTDYTRDLEEMQNVSREEYLASLRRKSSGFSRGISKYRALSSRWEPSYSRFAGSDYFNSMHYGAGDDSAAESEYASGFCIERKIDLTGHIKWWGSNKSRQPDAGTRLSEEKRHGFAGDICSEPKTLEQKVQPTEPYQMPELGRSHNEKKHRSSAVSALSILSQSAAYKSLQEKASKKQENSTDNDENENKNTVNKLDHGKAVEKSSNHDGGSDRVDIEIGTTGALSLQRNIYPLTPFLSAPLLTAYNTVDPSLVDPVLWTSLVPMLSAGLSCPTQVTKTETSSSYTIFQPEG

SEQ ID NO: 12 - protein, 440 aa, Ricinus communis
MASSSSDPGLKPELGGGSGGESSEAVIANDQLLLYRQLKKPKKERGCTAKERISKMPPCTAGKRSSIYRGVTRHRWTGRYEAHLWDKSTWNQNQNKKGKQGAYDDEEAAARAYDLAALKYWGPGTLINFPVTDYSRDLEEMQNVSREEYLASLRRKSSGFSRGISKYRGLSSQWDSSFGRMPGSEYFSSINYGAADDPAAESEYVGSLCFERKIDLTSYIRWWGFNKTRESVSKSSDERKHGYGEDISELKSSEWAVQSTEPYQMPRLGMPDNGKKHKCSKISALSILSHSAAYKNLQEKASKKQENCTDNDEKENKKTNKMDYGKAVEKSTSHDGSNERLGAALGMSGGLSLQRNAYQLAPFLSAPLLTNYNAIDPLVDPILWTSLVPVLPAGFSRNSEVGMGLQIVSCHKDRDKFNLYLLSAGGVSTFLLLVVHWRFC

SEQ ID NO: 13 - protein, 428 aa, Lupinus angustifolius; identical to SEQ ID NO: 11

SEQ ID NO: 14 - protein, 11 aa, synthetic construct
  REGION 1..11: note = conserved sequence
  VARIANT 4: note = Threonine (T) or Serine (S)
RGVXRHRWTGR

SEQ ID NO: 15 - protein, 8 aa, synthetic construct
  REGION 1..8: note = conserved sequence
  VARIANT 1: note = Phenylalanine (F) or Tyrosine (Y)
XEAHLWDK

SEQ ID NO: 16 - protein, 9 aa, synthetic construct
  REGION 1..9: note = conserved sequence
DLAALKYWG

SEQ ID NO: 17 - protein, 8 aa, synthetic construct
  REGION 1..8: note = conserved sequence
  VARIANT 2: note = X can be any amino acid
  VARIANT 5: note = Serine (S) or Alanine (A)
  VARIANT 8: note = X can be any amino acid
SXGFXRGX

SEQ ID NO: 18 - protein, 14 aa, synthetic construct
  REGION 1..14: note = conserved sequence
  VARIANT 3: note = Histidine (H) or Glutamine
  VARIANT 6: note = Arginine (R) or Lysine (K)
  VARIANT 13: note = Arginine (R) or Lysine (K)
HHXNGXWEARIGXV

SEQ ID NO: 19 - protein, 9 aa, synthetic construct
  REGION 1..9: note = conserved sequence
  VARIANT 7: note = X is any amino acid
QEEAAAXYD

SEQ ID NO: 20 - DNA, 11142 bp, synthetic construct
  misc_feature 1..11142: note = TDNA sequence
  source 1..11142: mol_type = other DNA
tcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccctagaatctaattattctattcagactaaattagtataagtatttttttaatcaataaataataattaataatttattagtaggagtgattgaatttataatatattttttttaatcatttaaagaatcttatatctttaaattgacaagagttttaaatggggagagtgttatcatatcacaagtaggattaatgtgttatagtttcacatgcattacgataagttgtgaaagataacattattatatataacaatgacaatcactagcgatcgagtagtgagagtcgtcttattacactttcttccttcgatctgtcacatggcggcggcccgaattctcacacaaggtagttgcaagacactgaagtggtggtagtggtagtagaagaagcagaatcggtagaaaggcaagacaatggagaagatgaagatggtggagattctcttcccacaacgcagcaatcaaggttttcaaggttaaggcactcgtgctttccatcatcgaacatgaagtcgatgttatcctcgaaagcaagctcgttgaagagttctgggtactcaattgggttctcgttagcaaggttttgatcggtaaggaatggggagaatccagtatccatcatgcagaagttccaagcaagttcgttgttatctccgcacctatccatttccatgatggtggaagaatcaatgcagcagttaacaacggcagcttcctcagaatatcccacaatttcagcctcttgttgctcagccttctcttcctctttttcttcttcctcttgaggtggttcctcaacgtattgttgcttaacctcttccctaggttcctctttagcttctctagtctcaacctcttgcttagcctcaacaagaataccctcttgatggttagcctggttaactgggaatgggaaaacgcccttcttcttaagcctgtcgatgtagttggagatatcgaagttggtaacagcgttagcacctctgtactcaatagcagccatatcataagcagctgcagcctcttcttgagtgttgtaagttccgaggtagaggtacttgtttccgaaaactcttccaatcctagcttcccatcttccgttatgatgatgcctagcaactcccctatacttagaaactcccctagagaatccagatgactgccttctaagggaagcaagatactcttctttggtcaccctctgcatctcttcaagttctttggtgtaagtctcagctgggaagttaagaatggtatctgggccccaatacttaagagcagcaagatcataggtatgagcagcagcctcttcagaatcataagctccaaggtaaacctgcttgcccttcttgttttggatggagttccaagaggacttatcccaaaggtgagcttcgaatcttccagtccatctatgcctagtaacacctctgtagatagatgaccttctggtagaagctggagaagttgggttatgagacttatcgccagatggagatgacttcttagccctcttagctctctttggtcttggagcttcagattgaattgggctagaggtagtagtagaagaggacactgaagaagatggagaactagagcaggtagaggtagtgagcctcttcttcatgaattctgttcttctttactctttgtgtgactgaggtttggtctagtgctttggtcatctatatataatgataacaacaatgagaacaagctttggagtgatcggagggtctaggatacatgagattcaagtggactaggatctacaccgttggattttgagtgtggatatgtgtgaggttaattttacttggtaacggccacaaaggcctaaggagaggtgttgagacccttatcggcttgaaccgctggaataatgccacgtggaagataattccatgaatcttatcgttatctatgagtgaaattgtgtgatggtggagtggtgcttgctcattttacttgcctggtggacttggccctttccttatggggaatttatattttacttactatagagctttcataccttttttttaccttggatttagttaatatataatggtatgattcatgaataaaaatgggaaatttttgaatttgtactgctaaatgcataagattaggtgaaactgtggaatatatatttttttcatttaaaagcaaaatttgccttttactagaattataaatatagaaaaatatataacattcaaataaaaatgaaaataagaactttcaaaaaacagaactatgtttaatgtgtaaagattagtcgcacatcaagtcatctgttacaatatgttacaacaagtcataagcccaacaaagttagcacgtctaaataaactaaagagtccacgaaaatattacaaatcataagcccaacaaagttattgatcaaaaaaaaaaaacgcccaacaaagctaaacaaagtccaaaaaaaacttctcaagtctccatcttcctttatgaacattgaaaactatacacaaaacaagtcagataaatctctttctgggcctgtcttcccaacctcctacatcacttccctatcggattgaatgttttacttgtaccttttccgttgcaatgatattgatagtatgtttgtgaaaactaatagggttaacaatcgaagtcatggaatatggatttggtccaagattttccgagagctttctagtagaaagcccatcaccagaaatttactagtaaaataaatcaccaattaggtttcttattatgtgccaaattcaatataattatagaggatatttcaaatgaaaacgtatgaatgttattagtaaatggtcaggtaagacattaaaaaaatcctacgtcagatattcaactttaaaaattcgatcagtgtggaattgtacaaaaatttgggatctactatatatatataatgctttacaacacttggatttttttttggaggctggaatttttaatctacatatttgttttggccatgcaccaactcattgtttagtgtaatactttgattttgtcaaatatatgtgttcgtgtatatttgtataagaatttctttgaccatatacacacacacatatatatatatatatatatattatatatcatgcacttttaattgaaaaaataatatatatatatatagtgcattttttctaacaaccatatatgttgcgattgatctgcaaaaatactgctagagtaatgaaaaatataatctattgctgaaattatctcagatgttaagattttcttaaagtaaattctttcaaattttagctaaaagtcttgtaataactaaagaataatacacaatctcgaccacggaaaaaaaacacataataaatttggggcccctagaatctaattattctattcagactaaattagtataagtatttttttaatcaataaataataattaataatttattagtaggagtgattgaatttataatatattttttttaatcatttaaagaatcttatatctttaaattgacaagagttttaaatggggagagtgttatcatatcacaagtaggattaatgtgttatagtttcacatgcattacgataagttgtgaaagataacattattatatataacaatgacaatcactagcgatcgagtagtgagagtcgtcttattacactttcttccttcgatctgtcacatggcggcggcccgcggccgcttcattactcgagccaggaggatggatcgatgctggtctgagaccctgctaccggttgctgactgaactgctcggcacggtccttcatttcacgggccttgctcgccaactttgtcttggccgactccaactgatccgctccgggtggatgtttccccgtcaggtaacggtagatccaggacagcacagacagagcggcaacaccaaatcccccgcttgccagaaaacccgctcccaacaggaagatggtgatgactgcagatcagaaaaactcagattaatcgacaaattcgatcgcacaaactagaaactaacaccagatctagatagaaatcacaaatcgaagagtaattattcgacaaaactcaaattatttgaacaaatcggatgatatctatgaaaccctaatcgagaattaagatgatatctaacgatcaaacccagaaaatcgtcttcgatctaagattaacagaatctaaaccaaagaacatatacgaaattgggatcgaacgaaaacaaaatcgaagattttgagagaataaggaacacagaaatttacctgcagggaccagtacaggcgagaagatcaccaggagaggtgtggcgattgtcagcgcaatgaccgttccagccagggtcaacccggataacaccaacaggctacctccggcagtaaccgcggtcgctgcctttacaacacgctgagcacgcggttgcagttgcaagtggggggcacgtgtttgttgctgctgcccgtagtgctctgccatggttttttttaacggagcaagcggccgctgttcttctttactctttgtgtgactgaggtttggtctagtgctttggtcatctatatataatgataacaacaatgagaacaagctttggagtgatcggagggtctaggatacatgagattcaagtggactaggatctacaccgttggattttgagtgtggatatgtgtgaggttaattttacttggtaacggccacaaaggcctaaggagaggtgttgagacccttatcggcttgaaccgctggaataatgccacgtggaagataattccatgaatcttatcgttatctatgagtgaaattgtgtgatggtggagtggtgcttgctcattttacttgcctggtggacttggccctttccttatggggaatttatattttacttactatagagctttcataccttttttttaccttggatttagttaatatataatggtatgattcatgaataaaaatgggaaatttttgaatttgtactgctaaatgcataagattaggtgaaactgtggaatatatatttttttcatttaaaagcaaaatttgccttttactagaattataaatatagaaaaatatataacattcaaataaaaatgaaaataagaactttcaaaaaacagaactatgtttaatgtgtaaagattagtcgcacatcaagtcatctgttacaatatgttacaacaagtcataagcccaacaaagttagcacgtctaaataaactaaagagtccacgaaaatattacaaatcataagcccaacaaagttattgatcaaaaaaaaaaaacgcccaacaaagctaaacaaagtccaaaaaaaacttctcaagtctccatcttcctttatgaacattgaaaactatacacaaaacaagtcagataaatctctttctgggcctgtcttcccaacctcctacatcacttccctatcggattgaatgttttacttgtaccttttccgttgcaatgatattgatagtatgtttgtgaaaactaatagggttaacaatcgaagtcatggaatatggatttggtccaagattttccgagagctttctagtagaaagcccatcaccagaaatttactagtaaaataaatcaccaattaggtttcttattatgtgccaaattcaatataattatagaggatatttcaaatgaaaacgtatgaatgttattagtaaatggtcaggtaagacattaaaaaaatcctacgtcagatattcaactttaaaaattcgatcagtgtggaattgtacaaaaatttgggatctactatatatatataatgctttacaacacttggatttttttttggaggctggaatttttaatctacatatttgttttggccatgcaccaactcattgtttagtgtaatactttgattttgtcaaatatatgtgttcgtgtatatttgtataagaatttctttgaccatatacacacacacatatatatatatatatatatattatatatcatgcacttttaattgaaaaaataatatatatatatatagtgcattttttctaacaaccatatatgttgcgattgatctgcaaaaatactgctagagtaatgaaaaatataatctattgctgaaattatctcagatgttaagattttcttaaagtaaattctttcaaattttagctaaaagtcttgtaataactaaagaataatacacaatctcgaccacggaaaaaaaacacataataaatttgggcgcgccgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctgcgatcgcatggcgattttggattctgctggcgttactacggtgacggagaacggtggcggagagttcgtcgatcttgataggcttcgtcgacggaaatcgagatcggattcttctaacggacttcttctctctggttccgataataattctccttcggatgatgttggagctcccgccgacgttagggatcggattgattccgttgttaacgatgacgctcagggaacagccaatttggccggagataataacggtggtggcgataataacggtggtggaagaggcggcggagaaggaagaggaaacgccgatgctacgtttacgtatcgaccgtcggttccagctcatcggagggcgagagagagtccacttagctccgacgcaatcttcaaacagagccatgccggattattcaacctctgtgtagtagttcttattgctgtaaacagtagactcatcatcgaaaatcttatgaagtatggttggttgatcagaacggatttctggtttagttcaagatcgctgcgagattggccgcttttcatgtgttgtatatccctttcgatctttcctttggctgcctttacggttgagaaattggtacttcagaaatacatatcagaacctgttgtcatctttcttcatattattatcaccatgacagaggttttgtatccagtttacgtcaccctaaggtgtgattctgcttttttatcaggtgtcactttgatgctcctcacttgcattgtgtggctaaagttggtttcttatgctcatactagctatgacataagatccctagccaatgcagctgataaggccaatcctgaagtctcctactacgttagcttgaagagcttggcatatttcatggtcgctcccacattgtgttatcagccaagttatccacgttctgcatgtatacggaagggttgggtggctcgtcaatttgcaaaactggtcatattcaccggattcatgggatttataatagaacaatatataaatcctattgtcaggaactcaaagcatcctttgaaaggcgatcttctatatgctattgaaagagtgttgaagctttcagttccaaatttatatgtgtggctctgcatgttctactgcttcttccacctttggttaaacatattggcagagcttctctgcttcggggatcgtgaattctacaaagattggtggaatgcaaaaagtgtgggagattactggagaatgtggaatatgcctgttcataaatggatggttcgacatatatacttcccgtgcttgcgcagcaagataccaaagacactcgccattatcattgctttcctagtctctgcagtctttcatgagctatgcatcgcagttccttgtcgtctcttcaagctatgggcttttcttgggattatgtttcaggtgcctttggtcttcatcacaaactatctacaggaaaggtttggctcaacggtggggaacatgatcttctggttcatcttctgcattttcggacaaccgatgtgtgtgcttctttattaccacgacctgatgaaccgaaaaggatcgatgtcatgagcgatcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcagggcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgtccgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtcc

SEQ ID NO: 21 - DNA, 16749 bp, synthetic construct
  misc_feature 1..16749: note = vector sequence
  source 1..16749: mol_type = other DNA
gcttcctcgtgctttacggtatcgccgctcccgattcgcagcgcatcgccttctatcgccttcttgacgagttcttctgaaacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgtccgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcaccttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacaccccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgctggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcggatcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggcccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgtctgcgatcgctgaagttcctatacttttcagagaataggaacttcggaataggaacttcccatgggatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcacgctagcggataacaatttcacacagggatatcactagtaaaaggtaccgagctcctgcagtatcgatgcggccgcaaagtcgacgaattctcattagcagaactcaagatgctgatcctctggaacgttgaacttgagcttgtgttcctcgaaaagcttgcacaactctttgatgtaacgctggtgaagtctatcaacttcctctctagaaggctgaggagtcatttgaacctcgataggctttccaacgatagtagtgataggctgtctgaaaggcatgagtccgaaagagtattggaaaactccccttccatggaaaagtggaaggctgattcccataatcttttggagtctgttctggatccatctaagccaagttccaggagtgttctcaacctggttgaagaggttgttctctccgaatgagaagataggaacaagagcagcaccatgcataagagcaagtctgatgaatcccttacggttcttcaagagaagtctgtaagcaccaggtctagcatcaagagcctcttgagcacctccaacgatgatagcaagaaggtttccaccaccctttctgctaaggatgtgatcagcagaaactttctcgctagacacgagtccaccagacatgatgtaatctctgaagaatggagccctgaaccaaacggtaagcatcataaggtaggatctgattccagggaacaaagaggtgaatccagtagactcagtacagaggttaaggaaagcaccagcagcaagaacaccatgaggatggaatccagcaatgtagttacggctaggatcaagctcagcagtcttaacgagagacacagggaagtaatccttcatgtacttccagatggccaatcttctgaagaattggataggtctaccaccttgtctaggcttatcccaatccaagtaccaccaggtagcgtaaagaacagagaaaagccagaacctggtgaacaagagtccaacgaagataacgatgcagagttgagcaagagcaaggaatgagaaaacccactgaagaacagcgaaagtctgcaatcttctctcccaaggaacaagaagtggagcgaactcgaccatgaattcagtcccccgtgttctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatgggatccaagggccctagaatctaattattctattcagactaaattagtataagtatttttttaatcaataaataataattaataatttattagtaggagtgattgaatttataatatattttttttaatcatttaaagaatcttatatctttaaattgacaagagttttaaatggggagagtgttatcatatcacaagtaggattaatgtgttatagtttcacatgcattacgataagttgtgaaagataacattattatatataacaatgacaatcactagcgatcgagtagtgagagtcgtcttattacactttcttccttcgatctgtcacatggcggcggcccgaattctcacacaaggtagttgcaagacactgaagtggtggtagtggtagtagaagaagcagaatcggtagaaaggcaagacaatggagaagatgaagatggtggagattctcttcccacaacgcagcaatcaaggttttcaaggttaaggcactcgtgctttccatcatcgaacatgaagtcgatgttatcctcgaaagcaagctcgttgaagagttctgggtactcaattgggttctcgttagcaaggttttgatcggtaaggaatggggagaatccagtatccatcatgcagaagttccaagcaagttcgttgttatctccgcacctatccatttccatgatggtggaagaatcaatgcagcagttaacaacggcagcttcctcagaatatcccacaatttcagcctcttgttgctcagccttctcttcctctttttcttcttcctcttgaggtggttcctcaacgtattgttgcttaacctcttccctaggttcctctttagcttctctagtctcaacctcttgcttagcctcaacaagaataccctcttgatggttagcctggttaactgggaatgggaaaacgcccttcttcttaagcctgtcgatgtagttggagatatcgaagttggtaacagcgttagcacctctgtactcaatagcagccatatcataagcagctgcagcctcttcttgagtgttgtaagttccgaggtagaggtacttgtttccgaaaactcttccaatcctagcttcccatcttccgttatgatgatgcctagcaactcccctatacttagaaactcccctagagaatccagatgactgccttctaagggaagcaagatactcttctttggtcaccctctgcatctcttcaagttctttggtgtaagtctcagctgggaagttaagaatggtatctgggccccaatacttaagagcagcaagatcataggtatgagcagcagcctcttcagaatcataagctccaaggtaaacctgcttgcccttcttgttttggatggagttccaagaggacttatcccaaaggtgagcttcgaatcttccagtccatctatgcctagtaacacctctgtagatagatgaccttctggtagaagctggagaagttgggttatgagacttatcgccagatggagatgacttcttagccctcttagctctctttggtcttggagcttcagattgaattgggctagaggtagtagtagaagaggacactgaagaagatggagaactagagcaggtagaggtagtgagcctcttcttcatgaattcactagtgattaaattttgttggtgctttgagcatataacaagcatggtatatataggcacgtaaacaagttgagaaattttactttgagtttgacataaccaataaaagttagtgctgtttattacctcactcagtttgcaccgcaactgtcgttagtgatgtttacctttcctttttctattatttattagtattatataatatatatatatgtgtgatgagacttgaaattgtttagcaccgcaaatgtccttcttgaggggaggttttcttttgctgaggttggggtgtcacatacacccccctctatggactcaacgtccttgctgaggtttaccccacactacatgagatttttctagactcaatactatgatatttctcgccttatcggaattggttaaactcagttgaagttagggtcatatcgataaaattgacacatgatcgactctgatattaaacagattctctccctcgaacctcactcactttcctttttctattctttattagtattatataatagatccgttccaaccattcacgtacataagaagagagatatttttttttaatggactaacatgacaaataaaacaaacaaaggagtaatgatcactacaacaaattagattatgagggacaaataatttcatcatctataaatcatgtttcgtcactaaaaattttgtgtgacgaaaaagatttcgtcaatcagttgtcactaaaaatatacaaagacgatttaatgatgtttacctttccttttctattctttattagtattatataataaatatatgtgtgatgagacttgaaattgtttagcaccgcaaatgtccttgttgaagggaggttttcttttgctgaggttggggtgtcacatacaccccctctatggactgaacgtcctttttgaggtttattttacactgcatgagatttttctagattcaacattatgatttctagactcaacactacgatcgtcactaaagactattttttatatataaaaaaaatactttgtccttaaatgtataaattagggataaatttattattataaaaaaggttaataattttgtgattaaatctattattttgtcactgaaagtgtttgcttttaccgacgacatatatgtcactaaatattatcataagtagtgacaattacaattgtcacaaaataaaaaaaattattcatattcaacaaaaaagggtactacgacaatacattttttgtcactgaaagtaatcaagttgtgataaattaatttatttaatgacaaaaatatttgtatcaaaattcacccatgatcatataataaaaataactaaaattatactaaagcataaatgacaagaaaatctaactaaaacatatcaaatattactcctaaacaaagacatataagtaaaaatttcttccaaagtatcaataacgtggtgacacatagcttgcaatcaatcttgcttcaattttcaccttttatacctgtaaaaagaaagagaaaataaaacaatgatttaaaaatcgaattcccgcggcccctagaatctaattattctattcagactaaattagtataagtatttttttaatcaataaataataattaataatttattagtaggagtgattgaatttataatatattttttttaatcatttaaagaatcttatatctttaaattgacaagagttttaaatggggagagtgttatcatatcacaagtaggattaatgtgttatagtttcacatgcattacgataagttgtgaaagataacattattatatataacaatgacaatcactagcgatcgagtagtgagagtcgtcttattacactttcttccttcgatctgtcacatggcggcggcccgcggccgcttcattactcgagccaggaggatggatcgatgctggtctgagaccctgctaccggttgctgactgaactgctcggcacggtccttcatttcacgggccttgctcgccaactttgtcttggccgactccaactgatccgctccgggtggatgtttccccgtcaggtaacggtagatccaggacagcacagacagagcggcaacaccaaatcccccgcttgccagaaaacccgctcccaacaggaagatggtgatgactgcagatcagaaaaactcagattaatcgacaaattcgatcgcacaaactagaaactaacaccagatctagatagaaatcacaaatcgaagagtaattattcgacaaaactcaaattatttgaacaaatcggatgatatctatgaaaccctaatcgagaattaagatgatatctaacgatcaaacccagaaaatcgtcttcgatctaagattaacagaatctaaaccaaagaacatatacgaaattgggatcgaacgaaaacaaaatcgaagattttgagagaataaggaacacagaaatttacctgcagggaccagtacaggcgagaagatcaccaggagaggtgtggcgattgtcagcgcaatgaccgttccagccagggtcaacccggataacaccaacaggctacctccggcagtaaccgcggtcgctgcctttacaacacgctgagcacgcggttgcagttgcaagtggggggcacgtgtttgttgctgctgcccgtagtgctctgccatggaaattttgttggtgctttgagcatataacaagcatggtatatataggcacgtaaacaagttgagaaattttactttgagtttgacataaccaataaaagttagtgctgtttattacctcactcagtttgcaccgcaactgtcgttagtgatgtttacctttcctttttctattatttattagtattatataatatatatatatgtgtgatgagacttgaaattgtttagcaccgcaaatgtccttcttgaggggaggttttcttttgctgaggttggggtgtcacatacacccccctctatggactcaacgtccttgctgaggtttaccccacactacatgagatttttctagactcaatactatgatatttctcgccttatcggaattggttaaactcagttgaagttagggtcatatcgataaaattgacacatgatcgactctgatattaaacagattctctccctcgaacctcactcactttcctttttctattctttattagtattatataatagatccgttccaaccattcacgtacataagaagagagatatttttttttaatggactaacatgacaaataaaacaaacaaaggagtaatgatcactacaacaaattagattatgagggacaaataatttcatcatctataaatcatgtttcgtcactaaaaattttgtgtgacgaaaaagatttcgtcaatcagttgtcactaaaaatatacaaagacgatttaatgatgtttacctttccttttctattctttattagtattatataataaatatatgtgtgatgagacttgaaattgtttagcaccgcaaatgtccttgttgaagggaggttttcttttgctgaggttggggtgtcacatacaccccctctatggactgaacgtcctttttgaggtttattttacactgcatgagatttttctagattcaacattatgatttctagactcaacactacgatcgtcactaaagactattttttatatataaaaaaaatactttgtccttaaatgtataaattagggataaatttattattataaaaaaggttaataattttgtgattaaatctattattttgtcactgaaagtgtttgcttttaccgacgacatatatgtcactaaatattatcataagtagtgacaattacaattgtcacaaaataaaaaaaattattcatattcaacaaaaaagggtactacgacaatacattttttgtcactgaaagtaatcaagttgtgataaattaatttatttaatgacaaaaatatttgtatcaaaattcacccatgatcatataataaaaataactaaaattatactaaagcataaatgacaagaaaatctaactaaaacatatcaaatattactcctaaacaaagacatataagtaaaaatttcttccaaagtatcaataacgtggtgacacatagcttgcaatcaatcttgcttcaattttcaccttttatacctgtaaaaagaaagagaaaataaaacaatgatttaaaggcgcgccgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctgcgatcgcatggcgattttggattctgctggcgttactacggtgacggagaacggtggcggagagttcgtcgatcttgataggcttcgtcgacggaaatcgagatcggattcttctaacggacttcttctctctggttccgataataattctccttcggatgatgttggagctcccgccgacgttagggatcggattgattccgttgttaacgatgacgctcagggaacagccaatttggccggagataataacggtggtggcgataataacggtggtggaagaggcggcggagaaggaagaggaaacgccgatgctacgtttacgtatcgaccgtcggttccagctcatcggagggcgagagagagtccacttagctccgacgcaatcttcaaacagagccatgccggattattcaacctctgtgtagtagttcttattgctgtaaacagtagactcatcatcgaaaatcttatgaagtatggttggttgatcagaacggatttctggtttagttcaagatcgctgcgagattggccgcttttcatgtgttgtatatccctttcgatctttcctttggctgcctttacggttgagaaattggtacttcagaaatacatatcagaacctgttgtcatctttcttcatattattatcaccatgacagaggttttgtatccagtttacgtcaccctaaggtgtgattctgcttttttatcaggtgtcactttgatgctcctcacttgcattgtgtggctaaagttggtttcttatgctcatactagctatgacataagatccctagccaatgcagctgataaggccaatcctgaagtctcctactacgttagcttgaagagcttggcatatttcatggtcgctcccacattgtgttatcagccaagttatccacgttctgcatgtatacggaagggttgggtggctcgtcaatttgcaaaactggtcatattcaccggattcatgggatttataatagaacaatatataaatcctattgtcaggaactcaaagcatcctttgaaaggcgatcttctatatgctattgaaagagtgttgaagctttcagttccaaatttatatgtgtggctctgcatgttctactgcttcttccacctttggttaaacatattggcagagcttctctgcttcggggatcgtgaattctacaaagattggtggaatgcaaaaagtgtgggagattactggagaatgtggaatatgcctgttcataaatggatggttcgacatatatacttcccgtgcttgcgcagcaagataccaaagacactcgccattatcattgctttcctagtctctgcagtctttcatgagctatgcatcgcagttccttgtcgtctcttcaagctatgggcttttcttgggattatgtttcaggtgcctttggtcttcatcacaaactatctacaggaaaggtttggctcaacggtggggaacatgatcttctggttcatcttctgcattttcggacaaccgatgtgtgtgcttctttattaccacgacctgatgaaccgaaaaggatcgatgtcatgagcgatcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcagggcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgacc

SEQ ID NO: 22 - DNA, 137 bp, synthetic construct
  misc_feature 1..137: note = linker sequence
  source 1..137: mol_type = other DNA
atttaaatgcggccgcgaattcgtcgattgaggacgtccctactagacctgctggacctcctcctgctacttactacgattctctcgctgtgcatatggtcagtcatgcccgggcctgcaggcggccgcatttaaat

SEQ ID NO: 23 - DNA, 434 bp, synthetic construct
  misc_feature 1..434: note = hpRNAi
  source 1..434: mol_type = other DNA
gtgagcaatgaaccaagatttatcaataccgttacttttgatagcaaagagggttctcctactcttgttatggtccacggatatggtgcctctcagggtttcttctttcggaatttttatgcccttgcgaggcatttcaaagttattgctattgatcagcttggctggggtggttcaagcaggcctgacttcacatgcagaagtacagaagagactgaagattggtttattgattcctttgaggagtggcgcaaagccaaaaaccttagcaactttattttgcttgggcactcctttggagggtatgtcgctgcaaaatatgctctcaagcatccagagcatgttcagcagttgattctggtaggaccagctggatttacatcagagactgaacatatgtccgagcggcttacccagtttagagcaacatggaa

SEQ ID NO: 24 - DNA, 593 bp, synthetic construct
  misc_feature 1..593: note = hpRNAi
  source 1..593: mol_type = other DNA
actgctgatgctgtcaggcagtatctatggttgtttgaggagcataatgttcttgaattcctcgtacttgctggagatcatctatatcgaatggattatgaaaagttcattcaagcccacagagaaacagatgctgatattactgttgccgcactgccaatggatgaaaagcgagccactgcatttggtctcatgaagattgacgaagaaggacgcattattgaatttgcagagaaaccgaaaggagagcaattgaaagcaatgaaagtggatactaccattttaggtcttgatgatgagagagctaaagagatgccttttatcgcaagtatgggtatatatgtcattagcaaagatgtgatgttaaacttacttcgtgataagttccctggtgccaatgattttggcagtgaagttattcctggtgcaacttcgcttgggatgagagtgcaagcttatttatatgatggatactgggaagatattggtaccatcgaagctttctacaatgccaatttgggcattaccaaaaagccagtcccagattttagcttctatgaccgatcagctccaatctacacccaacctcgata

SEQ ID NO: 25-26 - skipped (2 entries)

SEQ ID NO: 27 - protein, 7 aa, synthetic construct
  REGION 1..7: note = probable lipid binding motif
  REGION 2..4: note = MISC_FEATURE - any amino acid
VXXXHGF

SEQ ID NO: 28 - protein, 584 aa, Arabidopsis thaliana
MNSMNNWLGFSLSPHDQNHHRTDVDSSTTRTAVDVAGGYCFDLAAPSDESSAVQTSFLSPFGVTLEAFTRDNNSHSRDWDINGGACNTLTNNEQNGPKLENFLGRTTTIYNTNETVVDGNGDCGGGDGGGGGSLGLSMIKTWLSNHSVANANHQDNGNGARGLSLSMNSSTSDSNNYNNNDDVVQEKTIVDVVETTPKKTIESFGQRTSIYRGVTRHRWTGRYEAHLWDNSCKREGQTRKGRQVYLGGYDKEEKAARAYDLAALKYWGPTTTTNFPLSEYEKEVEEMKHMTRQEYVASLRRKSSGFSRGASIYRGVTRHHQHGRWQARIGRVAGNKDLYLGTFGTQEEAAEAYDIAAIKFRGLSAVTNFDMNRYNVKAILESPSLPIGSSAKRLKDVNNPVPAMMISNNVSESANNVSGWQNTAFQHHQGMDLSLLQQQQERYVGYYNGGNLSTESTRVCFKQEEEQQHFLRNSPSHMTNVDHHSSTSDDSVTVCGNVVSYGGYQGFAIPVGTSVNYDPFTAAEIAYNARNHYYYAQHQQQQQIQQSPGGDFPVAISNNHSSNMYFHGEGGGEGAPTFSVWNDT

SEQ ID NO: 29 - DNA, 336 bp, synthetic construct
  misc_feature 1..336: note = inducible promoter
  source 1..336: mol_type = other DNA
tcgatagttgtgatagttcccacttgtccgtccgcatcggcatccgcagctcgggatagttccgacctaggattggatgcatgcggaaccgcacgagggcggggcggaaattgacacaccactcctctccacgcaccgttcaagaggtacgcgtatagagccgtatagagcagagacggagcactttctggtactgtccgcacgggatgtccgcacggagagccacaaacgagcggggccccgtacgtgctctcctaccccaggatcgcatccccgcatagctgaacatctatataaagacccccaaggttctcagtctcaccaacatcatcaacc

SEQ ID NO: 30 - DNA, 2466 bp, synthetic construct
  misc_feature 1..2466: note = inducer
  source 1..2466: mol_type = other DNA
atggccgacactagaagaaggcagaaccactcttgtgacccatgccgtaagggcaagagaagatgtgatgctccagagaaccgtaacgaggctaatgagaacggatgggtgtcatgctctaactgcaagaggtggaacaaggactgcaccttcaactggcttagctcccaaaggtctaaggctaagggtgctgctccaagagctaggactaagaaggctaggactgctactactacctccgagccttctacttccgctgctactattccaactcccgagtccgataatcacgatgctccaccagtgatcaactcccacgatgctttgccatcttggactcagggacttctttctcaccctggcgatctcttcgacttctcccattctgctattccagctaacgctgaggatgctgctaacgtgcaatctgatgctccattcccatgggatcttgctatcccaggcgatttctctatgggacagcaacttgagaagcccctctccccattgtctttccaggctgttcttcttccaccacactccccaaacactgatgatctcattcgtgagcttgaggaacagactaccgatccagattccgtgactgacactaactccgttcagcaagttgctcaggatggctctctttggtctgataggcagtctccactcctcccagaaaacagtttgtgcatggcttccgactctaccgctagaaggtatgctaggtccaccatgaccaagaacctcatgaggatctaccacgactccatggaaaacgccctttcttgctggcttactgagcacaactgcccatactccgaccagatttcttacctcccaccaaagcaaagggctgagtggggaccaaattggtctaacaggatgtgcattagggtgtgcaggctcgatagggtgtcaacttctcttagaggaagggctctctccgctgaagaagataaggctgctgctagggcacttcaccttgctattgtggctttcgcttctcagtggactcaacatgctcaaaggggagctggacttaacgtcccagctgatattgctgctgacgagcgttctattaggcgtaacgcttggaatgaggctaggcatgcacttcagcacactactggaatcccatccttcagggtgatcttcgccaacatcatcttcagcctcactcagtccgtgctcgatgatgatgagcaacatggaatgggagctaggctcgataagcttctcgagaatgatggtgctccagtgttcctcgagactgctaataggcagctctacaccttcaggcacaagttcgctaggatgcagagaaggggtaaggctttcaataggcttcctggtggatccgtggcttctactttcgctggaattttcgagactcccaccccctcatctgagtctccacaacttgatccagtggtggcttctgaggaacacaggtctactctgtctctcatgttctggctcgggatcatgttcgacactctgtctgctgctatgtaccagaggccacttgttgtgtccgatgaggactcccagatctcttctgcttctccaccaagaagaggtgccgagactcctattaaccttgattgctgggagccaccaaggcaggtcccatctaatcaagagaagtctgatgtgtggggcgacctgttccttaggacttctgattctttgcccgaccacgagtcccacactcaaatttctcaaccagctgctaggtggccatgcacttatgaacaagctgctgctgctctctcctctgctactcctgttaaggtgttgctttacaggcgtgtgactcagctccagactttgttgtataggggagcttctccagctaggcttgaggctgctattcagaggactctctacgtgtacaaccactggactgctaagtaccagccattcatgcaggattgcgttgccaaccatgagcttctcccatccaggatccagtcttggtacgtgatccttgatggacactggcaccttgctgctatgcttttggctgatgtgctcgagtccatcgacagggattcctactccgatatcaaccacatcgacctcgtgactaagctcaggcttgataacgctcttgctgtgtctgctctcgctaggtcatctcttagaggccaagaactcgatccaggcaaggcttctccaatgtacaggcacttccacgactcccttactgaggttgcattccttgttgagccatggactgtggtgctcatccactcatttgctaaggctgcttacatcctcctcgattgccttgatcttgatggtcagggaaacgctctcgctggataccttcaacttaggcagaactgcaactactgcatcagggctctccagttccttggccgtaagtctgatatggctgctctcgtggctaaggatcttgagaggggactcaacggaaaggtcgacagcttcctctaa

SEQ ID NO: 31 - protein, 208 aa, Arabidopsis thaliana
MTSSVIVAGAGDKNNGIVVQQQPPCVAREQDQYMPIANVIRIMRKTLPSHAKISDDAKETIQECVSEYISFVTGEANERCQREQRKTITAEDILWAMSKLGFDNYVDPLTVFINRYREIETDRGSALRGEPPSLRQTYGGNGIGFHGPSHGLPPPGPYGYGMLDQSMVMGGGRYYQNGSSGQDESSVGGGSSSSINGMPAFDHYGQYK

SEQ ID NO: 32 - protein, 278 aa, Zea mays
MDSSSFLPAAGAENGSAAGGANNGGAAQQHAAPAIREQDRLMPIANVIRIMRRVLPAHAKISDDAKETIQECVSEYISFITGEANERCQREQRKTITAEDVLWAMSRLGFDDYVEPLGAYLHRYREFEGDARGVGLVPGAAPSRGGDHHPHSMSPAAMLKSRGPVSGAAMLPHHHHHHDMQMHAAMYGGTAVPPPAGPPHHGGFLMPHPQGSSHYLPYAYEPTYGGEHAMAAYYGGAAYAPGNGGSGDGSGSGGGGGSASHTPQGSGGLEHPHPFAYK

SEQ ID NO: 33 - protein, 234 aa, Arabidopsis thaliana
MERGGFHGYRKLSVNNTTPSPPGLAANFLMAEGSMRPPEFNQPNKTSNGGEEECTVREQDRFMPIANVIRIMRRILPAHAKISDDSKETIQECVSEYISFITGEANERCQREQRKTITAEDVLWAMSKLGFDDYIEPLTLYLHRYRELEGERGVSCSAGSVSMTNGLVVKRPNGTMTEYGAYGPVPGIHMAQYHYRHQNGFVFSGNEPNSKMSGSSSGASGARVEVFPTQQHKY

SEQ ID NO: 34 - protein, 312 aa, Arabidopsis thaliana
MVDENVETKASTLVASVDHGFGSGSGHDHHGLSASVPLLGVNWKKRRMPRQRRSSSSFNLLSFPPPMPPISHVPTPLPARKIDPRKLRFLFQKELKNSDVSSLRRMILPKKAAEAHLPALECKEGIPIRMEDLDGFHVWTFKYRYWPNNNSRMYVLENTGDFVNAHGLQLGDFIMVYQDLYSNNYVIQARKASEEEEVDVINLEEDDVYTNLTRIENTVVNDLLLQDFNHHNNNNNNNSNSNSNKCSYYYPVIDDVTTNTESFVYDTTALTSNDTPLDFLGGHTTTTNNYYSKFGTFDGLGSVENISLDDFY

SEQ ID NO: 35 - protein, 321 aa, Brassica napus
MMADENVETKASTLIASVGHQGHGFGSGSGGHHGLSASVPLLGVNSKKRRMPRQRRSSSSFNLLSLPPPMPLSPHVPTPLSARKIDPRKLRFLFQKELKNSDVSSLRRMILPKKAAEAHLPALECKEGIPIRMEDLDGLHVWTFKYRYWPNNNSRMYVLENTGDFVNAHGLQLGDFIMVYLDLDSNNYVIQARKASEEEEEEEDVTIIEEDDVYTNLTKIENTVVNDLLIQDFNHHNDNSSNNNSNNNINNNKCSYYYPVIDDITTNTASFVYDTTTLTSNDSPLDFLGGHTTTTTNTYYSKFGSFEGLGSVENISLDDFY

SEQ ID NO: 36 - protein, 314 aa, Medicago truncatula
MMMDEGEGKKKVVVQKTEACGFMAGVEDELGFVNVKGDNNNGSGQRIHHDHGFVAAAFGTVHRKKRMARQRRSSSSTITIHLKNLPSSTTTTTTTTTSHVPISPIPPLFHSLPPAREIDHRRLRFLFQKELKNSDVSSLRRMVLPKKAAEAFLPVLESKEGILLSMDDLDGLHVWSFKYRFWPNNNSRMYVLENTGDFVSTHGLRFGDSIMVYQDNQNHNYVIQAKKACDQDEYMEEANDTINHIFVDDYEVNKSCFDVAYPAMNDTSMSFIYDTTISNDSPLDFLGGSMTNYSRIGSVETFGSVENLSLDDFY

SEQ ID NO: 37 - DNA, 3275 bp, Arabidopsis thaliana
  source 1..3275: mol_type = other DNA
ggttggctatatggtccaaattttgatttgcaatatgagattgcacagagagaacaatctttcattatgattaattattgtacaagtaacaaacaccaatctccgatatactttggctctttagcacattgttatgctagaagttagcggaaatctatatgttgttaaacgcagcgtttaaattgaacagtgtaatttaccttgaaattttaagactacatgctgtttagaatttcagatgaaaacatcttgatgttttagaaatccacgtgggaatagcgtaaaatcttatccaacgaacttattttggttttgttgtatttgtgcaagtcgtcacgctaatcgaaaaaagaaaagaaaaaaagaagccgtcatgatcggccatttctcggccgagtctgagtctgactctgcgtccgtgtcaccattatcagatcgagcctgtcttatctcgttgcgattccctatgcaaaaatcttcttcttttttttattcccccatttatctctgatctcttctctcttctcaagtaaacctctctgcttcacgtctcttcttttcttgtcgattttccccagataatcagttgaaaacacacccaaattcatcttcgaatcaataatggatataagtaatgaggctagtgtcgatcccttttcgattggaccatcatctatcatgggtcgaaccattgctttcagagtcttgttctgtagatcaatgtcacagcttaggcgtgatctctttcggttcttgttgcattggtttcttagatttaagctgaccgtttcaccgtttgtgtcgtggtttcatcctcggaaccctcaagggattttagcggtggttacaatcattgcctttgtgttgaaacgatacacgaatgtgaaaataaaggcggaaatggcttaccggaggaagttttggaggaatatgatgcggacggctttgacttatgaggaatgggctcatgctgctaagatgttagagaaggaaacaccaaagatgaatgaatctgatctttatgatgaagagttggttaagaacaagcttcaggagcttcgtcatcgtcgccaagaaggctcacttagagacattatgttttgtatgagagctgatttggtgaggaatctcggtaatatgtgtaattcggagcttcataaaggtagacttcaggttcctagacatatcaaagagtacattgatgaggtgtctactcagttgagaatggtttgtaactctgattcagaggagctttctttagaagagaagctttcttttatgcatgaaacacggcatgcctttggtagaacggctttgcttttgagtggtggggcttctcttggtgcgtttcatgttggtgtggttaggactttggttgagcataagcttttacctcgaataattgctggttctagtgttggatccatcatttgtgctgttgtggcctcaaggtcttggccagaactacagagtttctttgagaattctttgcattctttacagttctttgatcagctcggaggcgtgttctcaatagtgaaacgggtaatgacacaaggggctctacacgatatcagacagttgcaatgtatgcttagaaacctcacaagcaatctcacattccaagaagcttatgacatgacaggaaggattctcgggatcaccgtttgctccccaagaaagcatgaacctcctcggtgtcttaactatttgacttcgcctcatgtggttatatggagcgcagtgactgcttcttgtgcttttcctggtctctttgaagctcaagagctaatggctaaagatcgaagtggagagatcgtaccgtatcatccacctttcaatttggatccagaagtaggcactaaatcatcatctggacgccggtggagagatggtagtttggaggttgatttaccaatgatgcagcttaaagaactgttcaatgtcaatcattttattgtgagccaagccaatcctcacattgctccattactgcgtctaaaggatttagttcgagcttatggtggtagattcgcagctaagctcgcgcatctagtggagatggaggtcaaacatagatgcaaccaggtattagagctcggttttcctctcggtggactcgcaaagctttttgctcaggagtgggaaggtgatgttacagttgtaatgcctgctactcttgctcagtactcgaagattatacaaaatccgactcatgtcgagcttcagaaagcggctaaccaaggaagaagatgcacttgggagaagctctcagccataaaatcaaactgcgggatcgagcttgcgcttgatgattctgtagctattcttaaccatatgcggaggctcaagaaaagtgcggagagagccgccactgccacgtcttcgtctcatcacggattggcttcaaccaccagattcaatgcttcaagaagaatcccatcttggaacgtccttgccagagagaactcaacaggctcactggatgatctagtcactgacaataacctccacgcttcttcgggcaggaatttaagcgacagtgaaacagagagcgtggagttgagttcttggacaagaactggtggacctttaatgagaacagcttctgctaataagttcattgattttgttcagagtcttgatatcgacattgcattggtcagaggatttagtagcagtcccaattctccagcagttcctcctggtggctcgtttactccaagcccgagatccatagcggctcattcggatatcgaatcaaacagcaatagcaacaatcttggaacaagcacttcaagcataacagttactgaaggtgatcttctacagcctgagagaacgagtaacggatttgtgttaaacgtcgttaaaagagagaacttgggaatgccatcgattgggaaccaaaatacagagttaccagagagtgtacagctcgatataccggagaaggagatggattgtagctctgtatcagaacacgaagaagatgataacgacaatgaagaagaacataacggctcgagtctggttactgtttcttcagaagattccggtttacaagaaccggtgtctggtagtgttatagatgcttagagtgtgattgattcaagtgagtatagattcttaattaaatttgcagagtttccaaagggtttagtgcaccacttgtgtatgtttgtattgcttattgtttgaaattcatttgtgaaatcgaaatatatctgtaaattcagaaaatattctctcatccattacaaaatatttgagtc

SEQ ID NO: 38 - DNA, 2724 bp, Sorghum bicolor
  source 1..2724: mol_type = other DNA
atggatgacatcgccagcgaggcgccggtgggggcgttcgccatcggcccgtccacggcgctgggccgcgccgtcgcgctccgggtgctgctctgcggctccgcggcgcgcctgcggcaccgcctggccgcggcgctccgcgccgcgctgcccgtcgcggcggcgtggctgcacccgcgcgacaacacgcgcgggatcctgctcgccgtctgcgccgtcgcgctcctgctgcggggccgacgcggcagggccgggctgcgggcgagggtgcagtccgcctaccgccgcaagttctggcggaacatgatgcgcgccgcgctcacctacgaggagtgggcgcacgcggcgcggatgctggagcgcgaggccgccccgcgccgcgccagcgacgccgacctctacgacgaggagctcgtccgcaataagctccgcgagctcaggcaccggcgccacgagggatcgctcagggacatcgtcttctgcatgcgcgcggacctgctcaggaacctcggcaatatgtgcaaccccgaactgcacaaagggaggctgcaggtgcctagactcataaaggaatacattgaggaagtatctactcaactgaaaatggtctgtgattctgattcagatgagttgcctcttgaagagaaactcgcatttatgcatgagacaaggcatgcctttggtagaacagccttgctgctaagtggaggtgcttcattgggatcctttcatgtgggtgttgttaaaaccttggtagagcataaacttttgccaaggataatttcaggatcaagtgttggctcgataatgtgttctatagtagcaacaagatcatggcctgagctggagagcttttttgaagagtggcattccctgaaattttttgatcagatgggtggaatctttcctgtggttaaaagaattttgacgcaaggcgctgttcatgatataaggcacttgcaggtgcttttgagaaaccttaccagcaatttgacatttcaagaagcttatgacatgactggtcggattcttgttgtcaccgtgtgttctccaaggaagcatgagccgcctcgatgcctaaactatttaacatcacctcatgttcttatctggagtgcagtaacagcttcctgtgcttttcctggactttttgaggcccaagaattgatggcaaaagatagatttggtcaaaccattcctttccatgctccattcttattaggcatagaagaacgaactgttgctccaacccgccgctggagagatgggagcttagaaagcgatttacccatgaagcaattgaaggaactattcaatgtgaatcatttcatagtaagccaagccaatcctcacatagctccgctgttgagactaaaggaaatcgtcagggcttatggaggcagcttcgctgccaagcttgctgaacttgctgagatggaagtcaaacataggtgtaatcaagttttggaacttggatttcctctaggaggattagctaaattatttgctcaagattgggaaggcgatgttacagttgttatgccagccactcttgcgcagtattccaagatgatacagaacccatcttatgctgagcttcagaaggctgcgaatcaaggtaggagatgcacttgggaaaagctatcagccatcagggcaaattgtgctattgagcttgcactggatgaatgtgttgccctcctgaaccacttgcgtaggctaaagaggagtgcagaaagagcatccgcatcgcaaggatatggtccagcaatcaggttctgcccatctaggaggattccatcctggaatctcatagcaagagaaaattcaactggttctcttgaagaagaaatgcttacatctcctcaaggacctggaggagttgctggaacatctaccagaaaccagtatcctcagagaagtgcacatgagagcagcgacagtgaatctgagagtattgatttacactcttggacaagaagtggtggccctcttatgaggacaacctcagccaataaattcatcagctttgttcagaatcttgagatcgacacagaatccagaacaattccatcgagggaagacataactgatcttgtgacaccaaatgctggtaccttggcagctcatgcagtgagtagagaagcaatcgataggagcttggacaattcagctttagatatccatgataccagtacccctagatcgacatttggcccttcaacaagtattgtggtttctgaaggtgacttgttgcagcctgaaaagattgaaaatggtattttgtttaatgttgtaaggagggatactctgctcgggtctagtagtggagttgagtctcaaggatctcctcgggaaccagatgttgaaacagtacagacggagtgccttgatggcgtgtctacttctgatgatgatgatgacaaggaactaaatgccattgatgatggaggaactagtcccatgagcagaaataatctacaacatcaggggtcctcactggaagaaaaattataccatccctcttccttaaattctgaagacgagacaaacacaaacaaaccagaagctgcatcgatttttgatatatgtacagatatgcatccggcatctattagcctacctgaagggtcttcagaaaagacagaactggaaacaacaaagattcctgatgacaattcagctgttatgaatgatgaagttgcctcaggtgctggtaactaa

SEQ ID NO: 39 - DNA, 3470 bp, Nicotiana benthamiana
  source 1..3470: mol_type = other DNA
gttatctgatccaaacttctgactttttctattttccgaatccctatgttttttaataaatccatctctgccattgcagtgatatattcatttattgttatcaccttcttcatttattggtccctctgtgttttccatatattgaaggagaaaacattaactttatgcgattttgtagtttttctggttgattcctacaaccccttttgacattgatcttgtgggttacaaaaaacattgaatctttatgtcaaaatttgatctttgtatttcattttaaattgaaatttgatttttgggggtattaaggattcttttgtcggttgattttgtgccttttttgccaagttcttgtcggtctctgagctgaatttccataatttgacaaaaagaaaaggctaaagcagaaaggttgggagtttctttctttgactttcagaaactaaggtattttctttgatctaattcttgttaatatctggttcaatctgattccgttgaatcttgtgaatagcctttgtttccctattgtcagaaaattatttccttttcactttcctcgactctcagaagttagtacaatctttgttctgctaaatcttgtgaataacctttagcttagagttttaggtatctgtatattgggttctcttaacatttagcctagaagccttctctaggattagtcccccttttcattgagatggatataagtaatgaggctacaattgacttcttttccattggacctactacgatattgggtcgaacaatcgcctttagagtgttgttctgtaaatcaatttcacaattgaagcatcacctatttcatttcttgatatattacttgtacaaattcaagaatggtttgtcatactacttgacacccttgatctcgtggttgcaccctcgtaatccacaaggaatattggcattggtaacgcttctcgccttcttgttgaggcgatacacgaatgtaaaaatcaaggctgagatggcctataggaggaagttttggaggaatatgatgagatctgcattgacttatgaggagtgggctcatgctgccaagatgctagataaagagacccctaaaatgaatgaggcagatctttatgatgtagaattagttcgaaataaactccaagagcttcgacatcgtaggcaagagggttctatgagggatatcatattctgtatgagagctgaccttgttaggaatcttggtaatatgtgtaatccagaacttcacaagggaaggcttcatgtgcctagactgattaaggattatattgatgaggtttcaactcagttgagaatggtatgcgactctgattcggaggagcttctcttggaagagaagcttgctttcatgcatgaaacaagacatgcctttggtaggacagctttgcttttaagtggaggtgcttctttaggagctttccatgtgggcgtggtgaaaacacttgtagaacacaaactgatgccacggataattgctggttcaagtgtcggctcgattatgtgctccatagttgcaactcgatcttggcctgagctccagagttttttcgaggactcctggcactctttgcaatttttcgatcagttgggtgggatttttactattttcaggagggtcatgacccagggtgctgtacatgagatcagacagctgcaggtgctgttacgtaatctcacgaataatcttactttccaagaagcctatgacatgactggtagagttctggggattactgtttgctcgcctaggaaacatgaacctcctagatgcttgaactacttgacttcacctcatgttgttatatggagtgccgttaccgcttcttgtgcctttcctggtctcttcgaagctcaagaacttatggcaaaggatagaagtggagatcttgttccatatcacccaccatttcatttgggtcctgatgccacttctagtgcatctgctcgtcgttggagggatggtagcttggaggttgatttgccaatgatgcagctaaaggagctcttcaatgtcaatcactttattgtgagccaggcgaatccgcatattgctccactgctgaggatcaaagagtttgtaagagcttatggaggcaactttgctgccaagcttgctcaacttacggaaatggaggtgaagcacagatgcaatcaggtattagaacttggttttcccttgggaggattagcaaagctttttgctcaagaatgggagggtgatgtaactgttgtaatgcctgccactctagctcagtactcaaaaatcatacagaatccctcgactctggagctgcaaaaagcagcaaatcaaggaagaaggtgcacttgggaaaaactctcagccatgaaagcaaactgtggaattgagcttgcacttgatgaatgcgttgctatactgaatcacatgcgtagactgaaaaggagtgctgagagggcggctgctgcttcacatggcttggcaagcactgtcagatttaacacttccagaagaattccttcttggaactgcattgcacgagagaactcaacaggctcccttgaagattttcttgcggatgttgctgcttcacatcatcaaggaggcagtggttcgggggcgcatgttaaccgtagttggcgaacgcaccggaatgcacatgatggtagtgacagtgagccggaaaatgtggaccttaattcttggacaagatcgggtggtcctttgatgaggacaacatcagctgataagtttattgactttgtccagaacttggaaattggttcgcgattgaacaaaggattgactattgacctcaacaatattattcctcagatggcaagcagggaccatttctccccgagcccaagggtaacaacacctgatagaagttcagatacagaatttgatcaaagagattttagttacagggtccctgcgagtagttcaagcattatggtaggcgaaggtgaccttctgcagcctgaaaggactaacagcggtattgtcttcaatgtggtaaggaaaggagacttgaccccatcgaacagaagccttgattcagaaaataatagttccgtgcaggatgcagttgctgagtgcgtgcaacttgaaagtccagaaaaggagatggatattagctcagtatcggaggatggtgagaatgatgttgggcaaggaagtagggtaaatgaagttgattgtagtaaaaatcgttcatcaatcggtgatggcaacgataagcaagttattgatacttgagagtttagctttgattattctacacaggccattcgaattattttttatactcaaatggagcttctttcagagctaacacactcagaattggggttgtaaatagtgcaagtagcaaatctgtaataaatgtttagtgtagtcatcacccttctactagttcaaagtggctcagttcaattcaaattcagaacttcgataattcatgttt

SEQ ID NO: 40 - DNA, 713 bp, Nicotiana benthamiana
  source 1..713: mol_type = other DNA
tgtatgagagctgaccttgttaggaatcttggtaatatgtgtaatccagaacttcacaagggaaggcttcatgtgcctagactgattaaggattatattgatgaggtttcaactcagttgagaatggtatgcgactctgattcggaggagcttctcttggaagagaagcttgctttcatgcatgaaacaagacatgcctttggtaggacagctttgcttttaagtggaggtgcttctttaggagctttccatgtgggcgtggtgaaaacacttgtagaacacaaactgatgccacggataattgctggttcaagtgtcggctcgattatgtgctccatagttgcaactcgatcttggcctgagctccagagttttttcgaggactcctggcactctttgcaatttttcgatcagttgggtgggatttttactattttcaggagggtcatgacccagggtgctgtacatgagatcagacagctgcaggtgctgttacgtaatctcacgaataatcttactttccaagaagcctatgacatgactggtagagttctggggattactgtttgctcgcctaggaaacatgaacctcctagatgcttgaactacttgacttcacctcatgttgttatatggagtgccgttaccgcttcttgtgcctttcctggtctcttcgaagctcaagaacttatggcaaaggatagaagtggaga

SEQ ID NO: 41 - DNA, 1500 bp, Arabidopsis thaliana
  source 1..1500: mol_type = genomic DNA
cgaaaaaagaagtagaatatatatatatatatatatatatatatatatatatatatattcgtgtggacatcataaatgcctaaatgataatagttgatttcgagttttattttcgttacttccaatcaaattctccttgcaccatatttatttttttactgtgagaacatatataagtatatattggaattacgtatccgagaggtttttgcatatttcgtttatttattttcgatatccacactactgtattattaaaaatttgaaaaattcaactagggcttttcatcttctctagaattattcgtttatttatgtcgatgtccacactattattaaaataaaacgagaggatatggttggatcatccaagtttcgtttatgactctttgttcatttacaaacgtttagttttccacttaagttttgaaaagagttaatttccaatatattcggcacagtttttcaagtgtattcatctgttttttttttttttggttggctatatggtccaaattttgatttgcaatatgagattgcacagagagaacaatctttcattatgattaattattgtacaagtaacaaacaccaatctccgatatactttggctctttagcacattgttatgctagaagttagcggaaatctatatgttgttaaacgcagcgtttaaattgaacagtgtaatttaccttgaaattttaagactacatgctgtttagaatttcagatgaaaacatcttgatgttttagaaatccacgtgggaatagcgtaaaatcttatccaacgaacttattttggttttgttgtatttgtgcaagtcgtcacgctaatcgaaaaaagaaaagaaaaaaagaagccgtcatgatcggccatttctcggccgagtctgagtctgactctgcgtccgtgtcaccattatcagatcgagcctgtcttatctcgttgcgattccctatgcaaaaatcttcttcttttttttattcccccatttatctctgatctcttctctcttctcaagtaaacctctctgcttcacgtctcttcttttcttgtcgattttccccagataatcaggtaaataaggctactttcttatttgatctggtggtctttgtgttgaaatctctgggttttctctgttgatttcaaagttctctctttttttttttgtttactgggtgctgtgaaaaatgatcttgtcaaagtctcctcttttcatcgaattgaaactctaattagaaaaaagatcataacttttattaaaaaaatgagtttgctttgcttaattttgcgaattgcttcatagattcattgattagcctatttggggtaacaaaaaaaagctgacacggtttcagattccaaaaatagatcatgactctgtttcttctctgcagaggttttaataaatatatgcttcttctcatgagttctcgttttttttgtcaccttcgcagttgaaaacacacccaaattcatcttcgaatcaata

SEQ ID NO: 42 - DNA, 2871 bp, synthetic construct
  misc_feature 1..2871: note = Nucleotide sequence of the complement of the pSSU-Oleosin gene in the T-DNA of pJP3502. In order (complementary sequences): Glycine max Lectin terminator 348nt, 3' exon 255nt, UBQ10 intron 304nt, 5' exon 213nt, SSU promoter
  source 1..2871: mol_type = other DNA
ggcccctagaatctaattattctattcagactaaattagtataagtatttttttaatcaataaataataattaataatttattagtaggagtgattgaatttataatatattttttttaatcatttaaagaatcttatatctttaaattgacaagagttttaaatggggagagtgttatcatatcacaagtaggattaatgtgttatagtttcacatgcattacgataagttgtgaaagataacattattatatataacaatgacaatcactagcgatcgagtagtgagagtcgtcttattacactttcttccttcgatctgtcacatggcggcggcccgcggccgcttcattactcgagccaggaggatggatcgatgctggtctgagaccctgctaccggttgctgactgaactgctcggcacggtccttcatttcacgggccttgctcgccaactttgtcttggccgactccaactgatccgctccgggtggatgtttccccgtcaggtaacggtagatccaggacagcacagacagagcggcaacaccaaatcccccgcttgccagaaaacccgctcccaacaggaagatggtgatgactgcagatcagaaaaactcagattaatcgacaaattcgatcgcacaaactagaaactaacaccagatctagatagaaatcacaaatcgaagagtaattattcgacaaaactcaaattatttgaacaaatcggatgatatctatgaaaccctaatcgagaattaagatgatatctaacgatcaaacccagaaaatcgtcttcgatctaagattaacagaatctaaaccaaagaacatatacgaaattgggatcgaacgaaaacaaaatcgaagattttgagagaataaggaacacagaaatttacctgcagggaccagtacaggcgagaagatcaccaggagaggtgtggcgattgtcagcgcaatgaccgttccagccagggtcaacccggataacaccaacaggctacctccggcagtaaccgcggtcgctgcctttacaacacgctgagcacgcggttgcagttgcaagtggggggcacgtgtttgttgctgctgcccgtagtgctctgccatggttttttttaacggagcaagcggccgctgttcttctttactctttgtgtgactgaggtttggtctagtgctttggtcatctatatataatgataacaacaatgagaacaagctttggagtgatcggagggtctaggatacatgagattcaagtggactaggatctacaccgttggattttgagtgtggatatgtgtgaggttaattttacttggtaacggccacaaaggcctaaggagaggtgttgagacccttatcggcttgaaccgctggaataatgccacgtggaagataattccatgaatcttatcgttatctatgagtgaaattgtgtgatggtggagtggtgcttgctcattttacttgcctggtggacttggccctttccttatggggaatttatattttacttactatagagctttcataccttttttttaccttggatttagttaatatataatggtatgattcatgaataaaaatgggaaatttttgaatttgtactgctaaatgcataagattaggtgaaactgtggaatatatatttttttcatttaaaagcaaaatttgccttttactagaattataaatatagaaaaatatataacattcaaataaaaatgaaaataagaactttcaaaaaacagaactatgtttaatgtgtaaagattagtcgcacatcaagtcatctgttacaatatgttacaacaagtcataagcccaacaaagttagcacgtctaaataaactaaagagtccacgaaaatattacaaatcataagcccaacaaagttattgatcaaaaaaaaaaaacgcccaacaaagctaaacaaagtccaaaaaaaacttctcaagtctccatcttcctttatgaacattgaaaactatacacaaaacaagtcagataaatctctttctgggcctgtcttcccaacctcctacatcacttccctatcggattgaatgttttacttgtaccttttccgttgcaatgatattgatagtatgtttgtgaaaactaatagggttaacaatcgaagtcatggaatatggatttggtccaagattttccgagagctttctagtagaaagcccatcaccagaaatttactagtaaaataaatcaccaattaggtttcttattatgtgccaaattcaatataattatagaggatatttcaaatgaaaacgtatgaatgttattagtaaatggtcaggtaagacattaaaaaaatcctacgtcagatattcaactttaaaaattcgatcagtgtggaattgtacaaaaatttgggatctactatatatatataatgctttacaacacttggatttttttttggaggctggaatttttaatctacatatttgttttggccatgcaccaactcattgtttagtgtaatactttgattttgtcaaatatatgtgttcgtgtatatttgtataagaatttctttgaccatatacacacacacatatatatatatatatatatattatatatcatgcacttttaattgaaaaaataatatatatatatatagtgcattttttctaacaaccatatatgttgcgattgatctgcaaaaatactgctagagtaatgaaaaatataatctattgctgaaattatctcagatgttaagattttcttaaagtaaattctttcaaattttagctaaaagtcttgtaataactaaagaataatacacaatctcgaccacggaaaaaaaacacataataaatttg

SEQ ID NO: 43 - protein, 362 aa, Arabidopsis thaliana
MLKLSCNVTDSKLQRSLLFFSHSYRSDPVNFIRRRIVSCSQTKKTGLVPLRAVVSADQGSVVQGLATLADQLRLGSLTEDGLSYKEKFVVRSYEVGSNKTATVETIANLLQEVGCNHAQSVGFSTDGFATTTTMRKLHLIWVTARMHIEIYKYPAWGDVVEIETWCQSEGRIGTRRDWILKDSVTGEVTGRATSKWVMMNQDTRRLQKVSDDVRDEYLVFCPQEPRLAFPEENNRSLKKIPKLEDPAQYSMIGLKPRRADLDMNQHVNNVTYIGWVLESIPQEIVDTHELQVITLDYRRECQQDDVVDSLTTTTSEIGGTNGSATSGTQGHNDSQFLHLLRLSGDGQEINRGTTLWRKKPSS

SEQ ID NO: 44 - protein, 367 aa, Arabidopsis thaliana
MLKLSCNVTDHIHNLFSNSRRIFVPVHRQTRPISCFQLKKEPLRAILSADHGNSSVRVADTVSGTSPADRLRFGRLMEDGFSYKEKFIVRSYEVGINKTATIETIANLLQEVACNHVQNVGFSTDGFATTLTMRKLHLIWVTARMHIEIYKYPAWSDVVEIETWCQSEGRIGTRRDWILKDCATGEVIGRATSKWVMMNQDTRRLQRVTDEVRDEYLVFCPPEPRLAFPEENNSSLKKIPKLEDPAQYSMLGLKPRRADLDMNQHVNNVTYIGWVLESIPQEIIDTHELKVITLDYRRECQQDDIVDSLTTSETPNEVVSKLTGTNGSTTSSKREHNESHFLHILRLSENGQEINRGRTQWRKKSSR

SEQ ID NO: 45 - protein, 412 aa, Arabidopsis thaliana
MVATSATSSFFPVPSSSLDPNGKGNKIGSTNLAGLNSAPNSGRMKVKPNAQAPPKINGKKVGLPGSVDIVRTDTETSSHPAPRTFINQLPDWSMLLAAITTIFLAAEKQWMMLDWKPRRSDMLVDPFGIGRIVQDGLVFRQNFSIRSYEIGADRSASIETVMNHLQETALNHVKTAGLLGDGFGSTPEMFKKNLIWVVTRMQVVVDKYPTWGDVVEVDTWVSQSGKNGMRRDWLVRDCNTGETLTRASSVWVMMNKLTRRLSKIPEEVRGEIEPYFVNSDPVLAEDSRKLTKIDDKTADYVRSGLTPRWSDLDVNQHVNNVKYIGWILESAPVGIMERQKLKSMTLEYRRECGRDSVLQSLTAVTGCDIGNLATAGDVECQHLLRLQDGAEVVRGRTEWSSKTPTTTWGTAP

SEQ ID NO: 46 - protein, 345 aa, Arabidopsis thaliana
MFIAVEVSPVMEDITRQSKKTSVENETGDDQSATSVVLKAKRKRRSQPRDAPPQRSSVHRGVTRHRWTGRYEAHLWDKNSWNETQTKKGRQVYLGAYDEEDAAARAYDLAALKYWGRDTILNFPLCNYEEDIKEMESQSKEEYIGSLRRKSSGFSRGVSKYRGVAKHHHNGRWEARIGRVFGNKYLYLGTYATQEEAAIAYDIAAIEYRGLNAVTNFDISRYLKLPVPENPIDTANNLLESPHSDLSPFIKPNHESDLSQSQSSSEDNDDRKTKLLKSSPLVAEEVIGPSTPPEIAPPRRSFPEDIQTYFGCQNSGKLTAEEDDVIFGDLDSFLTPDFYSELNDC

SEQ ID NO: 47 - protein, 303 aa, Arabidopsis thaliana
MAKVSGRSKKTIVDDEISDKTASASESASIALTSKRKRKSPPRNAPLQRSSPYRGVTRHRWTGRYEAHLWDKNSWNDTQTKKGRQVYLGAYDEEEAAARAYDLAALKYWGRDTLLNFPLPSYDEDVKEMEGQSKEEYIGSLRRKSSGFSRGVSKYRGVARHHHNGRWEARIGRVFATQEEAAIAYDIAAIEYRGLNAVTNFDVSRYLNPNAAADKADSDSKPIRSPSREPESSDDNKSPKSEEVIEPSTSPEVIPTRRSFPDDIQTYFGCQDSGKLATEEDVIFDCFNSYINPGFYNEFDYGP

SEQ ID NO: 48 - protein, 445 aa, Avena sativa
MKRSPPPAPPAAPPPPQPSPSSSSPACSPSPSSSSCPSSSDSSSIVIPRKRARTQKAASGKPKAKASAKRPKKDASRSSKETDANGAAAAAGKRSSIYRGVTRHRWTGRFEAHLWDKNCFTSVQNKKKGRQVYLGAYDTEDAAARAYDLAALKYWGSETILNFSVEDYAKEMPEMEAVSREEYLAALRRRSSGFSRGVSKYRGVARHHHNGRWEARIGRVLGNKYLYLGTFDTQEEAAKAYDLAAIEYRGANAVTNFDISCYLDQPQLLAQLQQGPQVVPALQEELQHDVQHDLQNDNAVQELNSGEVQMPGAMDEPIALDDSTECINTPFEFDFSVEENLWSPCMDYELDAILGNNTSNSANMNEWFNDSTFESNIGCLFEGCSNIDDCSSSKHCADLAAFDFFKEGDDNDFSNMEMEITPQANDVSCPPNDVSCPPKMITVCN

SEQ ID NO: 49 - protein, 420 aa, Sorghum bicolor
MDMERSQQQKSPTESPPPPSPSSSSSSVSADTVLPPPGKRRRAATTAKAKAGAKPKRARKDAAAAADPPPPPAAAAAGKRSSVYRGVTRHRWTGRFEAHLWDKHCLAALHNKKKGRQVYLGAYDSEEAAARAYDLAALKYWGPETLLNFPVEDYSSEMPEMEGVSREEYLASLRRRSSGFSRGVSKYRGVARHHHNGRWEARIGRVFGNKYLYLGTFDTQEEAAKAYDLAAIEYRGVNAVTNFDISCYLDHPLFLAQLQQEPQVVPALNQEAQPDQSETETIAQESVSSEAKTPDDNAEPDDNAEPDDIAEPLITVDDSIEESLWSPCMDYELDTMSRSNFGSSINLSEWFNDADFDSNIGCLFDGCSAVDEGGKDGVGLADFSLLEDFSLFEAGDGQLKDVLSDMEEGIQPPTMISVCN

SEQ ID NO: 50 - protein, 395 aa, Zea mays
MERSQRQSPPPPSPSSSSSSVSADTVLVPPGKRRRAATAKAGAEPNKRIRKDPAAAAAGKRSSVYRGVTRHRWTGRFEAHLWDKHCLAALHNKKKGRQVYLGAYDSEEAAARAYDLAALKYWGPETLLNFPVEDYSSEMPEMEAVSREEYLASLRRRSSGFSRGVSKYRGVARHHHNGRWEARIGRVFGNKYLYLGTFDTQEEAAKAYDLAAIEYRGVNAVTNFDISCYLDHPLFLAQLQQEPQVVPALNQEPQPDQSETGTTEQEPESSEAKTPDGSAEPDENAVPDDTAEPLTTVDDSIEEGLWSPCMDYELDTMSRPNFGSSINLSEWFADADFDCNIGCLFDGCSAADEGSKDGVGLADFSLFEAGDVQLKDVLSDMEEGIQPPAMISVCN

SEQ ID NO: 51 - protein, 430 aa, Triadica sebifera
MASSSSDPVLKAELGSSGGGCSSGGGGESSEAVIANDQLLLYRGLKKPKKERGCTAKERISKMPPCTAGKRSSIYRGVTRHRWTGRYEAHLWDKSTWNQNQNKKGKQVYLGAYDDEEAAARAYDLAALKYWGPGTLINFPVTDYTRDLEEMQNMSREEYLASLRRKSSGFSRGISKYRGLSSRWESSVGRMPGSEYFSSINYVDDPAAESEYVGSLCFERKIDLTSYIKWWGLNKTRQAESISKSAEETKPGCAEDIGGELKTTEWAIQPTEPYQMPRLGMPVHVKKHKGSKISALSVLSQSAAFKSLQEKASKKQENSTDNDENENKNTNTNKIDYGKAVETSASHDSSNERPVTALGMSGGLSLKRNVYQLTPFLSAPLLTNYGTIDQLVDPILWASLVPVLPTGLSRNPEVTKTETSSTYTFFRPEE

SEQ ID NO: 52 - DNA, 1531 bp, Solanum tuberosum
  source 1..1531: mol_type = genomic DNA
ttttaaatcattgttttattttctctttctttttacaggtataaaaggtgaaaattgaagcaagattgattgcaagctatgtgtcaccacgttattgatactttggaagaaatttttacttatatgtctttgtttaggagtaatatttgatatgttttagttagattttcttgtcatttatgctttagtataattttagttatttttattatatgatcatgggtgaattttgatacaaatatttttgtcattaaataaattaatttatcacaacttgattactttcagtgacaaaaaatgtattgtcgtagtacccttttttgttgaatatgaataattttttttattttgtgacaattgtaattgtcactacttatgataatatttagtgacatatatgtcgtcggtaaaagcaaacactttcagtgacaaaataatagatttaatcacaaaattattaaccttttttataataataaatttatccctaatttatacatttaaggacaaagtattttttttatatataaaaaatagtctttagtgacgatcgtagtgttgagtctagaaatcataatgttgaatctagaaaaatctcatgcagtgtaaaataaacctcaaaaaggacgttcagtccatagagggggtgtatgtgacaccccaacctcagcaaaagaaaacctcccttcaacaaggacatttgcggtgctaaacaatttcaagtctcatcacacatatatttattatataatactaataaagaatagaaaaggaaaggtaaacatcattaaatcgtctttgtatatttttagtgacaactgattgacgaaatctttttcgtcacacaaaatttttagtgacgaaacatgatttatagatgatgaaattatttgtccctcataatctaatttgttgtagtgatcattactcctttgtttgttttatttgtcatgttagtccattaaaaaaaaatatctctcttcttatgtacgtgaatggttggaacggatctattatataatactaataaagaatagaaaaaggaaagtgagtgaggttcgagggagagaatctgtttaatatcagagtcgatcatgtgtcaattttatcgatatgaccctaacttcaactgagtttaaccaattccgataaggcgagaaatatcatagtattgagtctagaaaaatctcatgtagtgtggggtaaacctcagcaaggacgttgagtccatagaggggggtgtatgtgacaccccaacctcagcaaaagaaaacctcccctcaagaaggacatttgcggtgctaaacaatttcaagtctcatcacacatatatatatattatataatactaataaataatagaaaaaggaaaggtaaacatcactaacgacagttgcggtgcaaactgagtgaggtaataaacagcactaacttttattggttatgtcaaactcaaagtaaaatttctcaacttgtttacgtgcctatatataccatgcttgttatatgctcaaagcaccaacaaaattta

SEQ ID NO: 53 - DNA, 1970 bp, Zea mays
  source 1..1970: mol_type = genomic DNA
ggtaccttttttcccagagataaatgtggaatagctctacaaacaaacggcatgatgctgacacttggatggcgaccttgcaatcccaagaactattgcatacggttgccagtcgacaaatatctacgccatgcatggctacggtcggaatacaccgtagcggcgggtaactcgccgataccgtccacgtgtccttggatgcccggtcgctgatacttctggtcttctggacatgcaccaagacaatcaagtgattcaaccttaatttaacataatataaataatacgtaacatccaactgacgtgttcacctatagagaatattccttctgattctactttcagaatgatgccgttgccgtgtatcgagcaagtactctcactcgaagtatcttatctcccacatccagcacaaaaatcttctgttcgtggcaaatcttgtggcggttgaacgaaagaatgctatataagtagctatagagaacgtattatgtgtaaaccaaccgttcagtgtaaatcgtgtgtaaatagtcatgttaattttttggcggcagatcaagtacaaactgtatgcctcggataaacatgtacaaaccacaacactggccactagatctatatccaacgttcataaccatccatccctctctgctgcactctgcaaacaagcacccccatctcgtagcaacatcttgtctccgacaagctctcgatgtagtggaggccctccaccgcaatatcctagtgtatgatgttggagaagcgactcctaaataatggtggcaagatgttgctaggtttgtagccatagcctcaatctaagatcatcccaagccatgggacctgattctacgaggcctacaaccaggcatgacacgtcgtctacccactcttgtgcatcatcggtcacttgatctgacttggttcctaaccacttaccctaggttccaaagccctaagtttctcgtatattgttagtcattcttagtgggagttttatgtgtatttcattcctgttaaatagcatgccaactaagcaaacatgatgatataatatgcaatctaataaaaagatatatgagtgggtttcataaaaaagggagagagtttcatgaggagtgaaactctgaatacagatactgatatgacagctttaaaagtagtgttatgaaatcatcattgagaaatggtattagcactcaatcgatttctacgctgtcaattgtcatgagcacaattttcacccaaagaggcacaccagcaatgtccgcttgtagtgtccgagacgttgctccatcgccgtcgtcttgtttctgtgcgctccattcaatgcggcaagtggctcaatcccaagcggtcgtcgcctcccagccccagcagcaaaatatcttcccatgcggccatgccttgaaaattggaatagattctctagattcaccgccgcgtcatcttcactactttctcactggcccaatcagcatctccttctccgagctcaatcatgctcagtcaagcgtcaccaatggcgtcacggttggttttgtcactgtctgcatgcaagggtattttgcttcgcaagtgtaaatggaaaatggatctaaacaactgcactgcaccaattttggaacgcggaaccgagagtctgtttgggttcgtttgaaacgcgctgatgtttctcattttttaatagatgtagttacctgatactatttaagttggacgatcaaacgactgtgtcaagtgtgattaagaaaagcatcgaaaataaaatttatcgccataaaaagttaaaaacagtggataatagtaggacctcataatagaaaaaattatcaaacggaatggaggggcccaacgcagtatatagcagccgggtggtgccggacatccgacgctcgtgccagcaggccattcttctcgccttactccctcacagaacccagtaaaatatcgccagtcccgccgtcgag

SEQ ID NO: 54 - DNA, 584 bp, Aeluropus littoralis
  source 1..584: mol_type = genomic DNA
cccaagcttgaccgatgcacacgctacctgccaaggctccctccatccgcactctgcatcgtcgcttcggcgtaaacttccacgtagtacttgtacgattctagctagacccagtgcgcccaccctaccgccggcgagcgggcccccatctcgcgccaggcttccatgcgggtccaccgtggaccagccctacgccgaaccgagcccatccctccaccctttcaccgccaagcgggacccgcgttggacctttccgcttggctggcccccaccagcgtccacgcgggccaacggcctcgcgaaatggatctccacacgacaaaccaaaacgagaagaaaataaatggaaaggaaagaaacggatcgccacgcgttccagaggcgtccgctaaccacccgattatgcttgcgcagcgtgcgtaacctcgtcgtggggttaatccgggtggccggatcgggaaagccacggcctttataacccatccctgccggatcgaaccggtaccggaaacaaaaacagggggagaaaaaaagttcttcgcgaggaaggaaaaggaaaagtcgcgtgccgtcctcgcccacag

SEQ ID NO: 55 - DNA, 928 bp, Agrobacterium rhizogenes
  source 1..928: mol_type = genomic DNA
ttagcgaaaggatgtcaaaaaaggatgcccataattgggaggagtggggtaaagcttaaagttggcccgctattggatttcgcgaaagcggcattggcaaacgtggagattgctgcattcaagatactttttctattttctggttaagatgtaaagtattgccacaatcatattaattactaacattgtatatgtaatatagtgcggaaattatctatgccaaaatgatgtattaataatagcaataataatatgtgttaatctttttcaatcgggaatacgtttaagcgattatcgtgttgaataaattattccaaaaggaaatacatggttttggagaacctgctatagatatatgccaaatttacactagtttagtgggtgcaaaactattatctctgtttctgagtttaataaaaaataaataagcagggcgaatagcagttagcctaagaaggaatggtggccatgtacgtgcttttaagagaccctataataaattgccagctgtgttgctttggtgccgacaggcctaacgtggggtttagcttgacaaagtagcgcctttccgcagcataaataaaggtaggcgggtgcgtcccattattaaaggaaaaagcaaaagctgagattccatagaccacaaaccaccattattggaggacagaacctattccctcacgtgggtcgctagctttaaacctaataagtaaaaacaattaaaagcaggcaggtgtcccttctatattcgcacaacgaggcgacgtggagcatcgacagccgcatccattaattaataaatttgtggacctatacctaactcaaatatttttattatttgctccaatacgctaagagctctggattataaatagtttggatgcttcgagttatgggtacaagcaacctgtttcctactttgttacca

SEQ ID NO: 56 - protein, 512 aa, Elaeis guineensis
MAVSKNPETLAPDQEPSKESDLRRRPASSPSSTAASPAVPDSSSRTSSSITGSWTTALDGDSGAGAVRIGDPKDRIGEANDIGEKKKACSGEVPVGFVDRPSAPVHVRVVESPLSSDTIFQQSHAGLLNLCVVVLIAVNSRLIIENLMKYGLLIGSGFFFSSRLLRDWPLLICSLTLPVFPLGSYMVEKLAYKKFISEPVVVSLHVILIIATIMYPVFVILRCDSPILSGINLMLFVSSICLKLVSYAHANYDLRSSSNSIDKGIHKSQGVSFKSLVYFIMAPTLCYQPSYPRTTCIRKGWVICQLVKLVIFTGVMGFIIEQYIDPIIKNSQHPLKGNVLNAMERVLKLSIPTLYVWLCVFYCTFHLWLNILAELLCFGDREFYKDWWNAKTIEEYWRMWNMPVHKWMLRHVYLPCIRNGIPKGVAMVISFFISAIFHELCIGIPCHIFKFWAFIGIMFQVPLVILTKYLQNKFKSAMVGNMIFWFFFSIYGQPMCVLLYYHDVMNRKVGTE

SEQ ID NO: 57 - protein, 74 aa, Glycine max
MADIDRSFDNNVSAVSTEKSSQVSDVEFSEAEEILIAMVYNLVGERWSLIAGRIPGRTAEEIEKYWTSRFSTSQ

SEQ ID NO: 58 - protein, 146 aa, Arabidopsis thaliana
MGSLQMQTSPESDNDPRYATVTDERKRKRMISNRESARRSRMRKQKQLGDLINEVTLLKNDNAKITEQVDEASKKYIEMESKNNVLRAQASELTDRLRSLNSVLEMVEEISGQALDIPEIPESMQNPWQMPCPMQPIRASADMFDC

SEQ ID NO: 59 - protein, 268 aa, Arabidopsis thaliana
MGRGKIEIKRIENANSRQVTFSKRRSGLLKKARELSVLCDAEVAVIVFSKSGKLFEYSSTGMKQTLSRYGNHQSSSASKAEEDCAEVDILKDQLSKLQEKHLQLQGKGLNPLTFKELQSLEQQLYHALITVRERKERLLTNQLEESRLKEQRAELENETLRRQVQELRSFLPSFTHYVPSYIKCFAIDPKNALINHDSKCSLQNTDSDTTLQLGLPGEAHDRRTNEGERESPSSDSVTTNTSSETAERGDQSSLANSPPEAKRQRFSV

SEQ ID NO: 60 - protein, 437 aa, Arabidopsis thaliana
MEFESVFKMHYPYLAAVIYDDSSTLKDFHPSLTDDFSCVHNVHHKPSMPHTYEIPSKETIRGITPSPCTEAFGACFHGTSNDHVFFGMAYTTPPTIEPNVSHVSHDNTMWENDQNQGFIFGTESTLNQAMADSNQFNMPKPLLSANEDTIMNRRQNNQVMIKTEQIKKKNKRFQMRRICKPTKKASIIKGQWTPEEDKLLVQLVDLHGTKKWSQIAKMLQGRVGKQCRERWHNHLRPDIKKDGWTEEEDIILIKAHKEIGNRWAEIARKLPGRTENTIKNHWNATKRRQHSRRTKGKDEISLSLGSNTLQNYIRSVTYNDDPFMTANANANIGPRNMRGKGKNVMVAVSEYDEGECKYIVDGVNNLGLEDGRIKMPSLAAMSASGSASTSGSASGSGSGVTMEIDEPMTDSWMVMHGCDEVMMNEIALLEMIAHGRL

SEQ ID NO: 61 - protein, 359 aa, Arabidopsis thaliana
MYHQNLISSTPNQNSNPHDWDIQNPLFSIHPSAEIPSKYPFMGITSCPNTNVFEEFQYKITNDQNFPTTYNTPFPVISEGISYNMHDVQENTMCGYTAHNQGLIIGCHEPVLVHAVVESQQFNVPQSEDINLVSQSERVTEDKVMFKTDHKKKDIIGKGQWTPTEDELLVRMVKSKGTKNWTSIAKMFQGRVGKQCRERWRNHLRPNIKKNDWSEEEDQILIEVHKIVGNKWTEIAKRLPGRSENIVKNHWNATKRRLHSVRTKRSDAFSPRNNALENYIRSITINNNALMNREVDSITANSEIDSTRCENIVDEVMNLNLHATTSVYVPEQAVLTWGYDFTKCYEPMDDTWMLMNGWN

SEQ ID NO: 62 - protein, 386 aa, Arabidopsis thaliana
MSKRPPPDPVAVLRGHRHSVMDVSFHPSKSLLFTGSADGELRIWDTIQHRAVSSAWAHSRANGVLAVAASPWLGEDKIISQGRDGTVKCWDIEDGGLSRDPLLILETCAYHFCKFSLVKKPKNSLQEAESHSRGCDEQDGGDTCNVQIADDSERSEEDSGLLQDKDHAEGTTFVAVVGEQPTEVEIWDLNTGDKIIQLPQSSPDESPNASTKGRGMCMAVQLFCPPESQGFLHVLAGYEDGSILLWDIRNAKIPLTSVKFHSEPVLSLSVASSCDGGISGGADDKIVMYNLNHSTGSCTIRKEITLERPGVSGTSIRVDGKIAATAGWDHRIRVYNYRKGNALAILKYHRATCNAVSYSPDCELMASASEDATVALWKLYPPHKSL

SEQ ID NO: 63 - protein, 292 aa, Arabidopsis thaliana
MEPPQHQHHHHQADQESGNNNNNKSGSGGYTCRQTSTRWTPTTEQIKILKELYYNNAIRSPTADQIQKITARLRQFGKIEGKNVFYWFQNHKARERQKKRFNGTNMTTPSSSPNSVMMAANDHYHPLLHHHHGVPMQRPANSVNVKLNQDHHLYHHNKPYPSFNNGNLNHASSGTECGVVNASNGYMSSHVYGSMEQDCSMNYNNVGGGWANMDHHYSSAPYNFFDRAKPLFGLEGHQEEEECGGDAYLEHRRTLPLFPMHGEDHINGGSGAIWKYGQSEVRPCASLELRLN

SEQ ID NO: 64 - protein, 453 aa, Brassica napus
MDLGSVTGNVNGSPSLKELRESKQDRSEFDGEDCLQQSSKLARTIAEDKHLPSSYAAAYSRPMSFHQGIPLARSASLLSSDSRRQEHMLSFSDKPEAFDFSKYVGLDNNKNSLSPFLHQLPPPYCRTPGGGYGSGGMMMSMQGKGPFTLTQWAELEQQALIYKYITANVPVPSSLLISIQKSFYPYRSFPPSSFGWGTFHLGFAGGKMDPEPGRCRRTDGKKWRCSKDAVPDQKYCERHINRGRHRSRKPVEVQPGQTAASKAAAVASRNTASQIPNNRVQNVIYPSTVNLPPKEQRNNNNSSFGFGHVTSPSLLTSSYLDFSSNQNKPEELKSDWTQLSMSIPVASSSPSSTAQDKTTLSPLRLDLPIQSQQETLEAVRKVNTWIPISWGNSLGGPLGEVLNSTTSSPTLGSSPTGVLQKSTFCSLSNSSSVTSPVADNNRNNNVDYFHYTT

SEQ ID NO: 65 - protein, 461 aa, Brassica napus
MDLGSVTGNVNGSPGLKELRGSKQDRSGFDGEDCLQQSSKLARTIAEDKHLPSSYAAYSRPMSFHQGIPLTRSASLLSSDSRRQEHMLSFSDKPEAFDFSKYVGLDNNKNSLSPFLHQLPPPYCRSSGGGYGSGGMMMSMQGKGPFTLTQWAELEQQALIYKYITANVPVPSSLLISIQKSFYPYRSFPPSSFGWGTFHLGFAGGKMDPEPGRCRRTDGKKWRCSKDAVPEQKYCERHINRGRHRSRKPVEVQPGQTAASKAVASRDTASQIPSNRVQNVIYPSNVNLQPKEQRNNDNSPFGFGHVTSSSLLTSSYLDFSSNQEKPSGNHHNQSSWPEELKSDWTQLSMSIPVASSSPSSTAQDKTALSPLRLDLPIQSQQETLESARKVNTWIPISWGNSLGGPLGEVLNSTTSSPTLGSSPTGVLQKSTFCSLSNSSSVTSPIADNNRNNNVDYFHYTT

SEQ ID NO: 66 - protein, 409 aa, Arabidopsis thaliana
MEARPVHRSGSRDLTRTSSIPSTQKPSPVEDSFMRSDNNSQLMSRPLGQTYHLLSSSNGGAVGHICSSSSSGFATNLHYSTMVSHEKQQHYTGSSSNNAVQTPSNNDSAWCHDSLPGGFLDFHETNPAIQNNCQIEDGGIAAAFDDIQKRSDWHEWADHLITDDDPLMSTNWNDLLLETNSNSDSKDQKTLQIPQPQIVQQQPSPSVELRPVSTTSSNSNNGTGKARMRWTPELHEAFVEAVNSLGGSERATPKGVLKIMKVEGLTIYHVKSHLQKYRTARYRPEPSETGSPERKLTPLEHITSLDLKGGIGITEALRLQMEVQKQLHEQLEIQRNLQLRIEEQGKYLQMMFEKQNSGLTKGTASTSDSAAKSEQEDKKTADSKEVPEEETRKCEELESPQPKRPKIDN

SEQ ID NO: 67 - DNA, 1173 bp, Sapium sebiferum L.
  source 1..1173: mol_type = genomic DNA
tgccaatagccagccaataaaacatctacacgttttcacacggcttttcatcacagccgttgtttttctcatctcactccgtgccttcatcttcatcctcttctcctctctctctgtctctatatgtatagaagcgttagatgtcttgcgttgttaaccaattcatttttcgctttctgcttcttctaatattataagaaagtttgattcttcttcttgtcaatctttgttcgcggcttttaacgatatccgctaaaggaaatttgaaatttcaattatggccgatggaaacgtcaattcgcaagaacagatggctaagcaggaggaacagaggctgaagtatttggagtttgtacaagtggctgcaatacatgctgtggtgaccttcacaaacctctatgtttatgccaaaaacaagtcgggtccattgaagcccggtgttgagactgttgaaggtacggtcaagagtgtggttggacctgtttatggcaagttccatgatgttcccattgaggttctcaagtttgtcgatcgcaagattgatcaatctgtaagcagcctagacagccgtgtgcctccagttgtgaagcagttatcggcccaagcattttcagtggctcgcgaagccccagtggctgctcgtgctgtggcttctgaagtgcagactgctggagtgaaggaaactgcatctgggttggcaagaactctgtacttcaaatatgaacccaaggccaaggagctatacaccaagtatgaaccaaaagcggaacagtgtgctgcctctgcctggcgtaagctcaatcaactcccagtcttccctcatgtagctcaggttgttatgccaacagcagcttattgttctgaaaagtacaaccaggcagtacttaccaccgctgagaaaggatacagagtgtcctcttatttgccttttgtgcccactgagagaattgctaagttgtttaggaatgaggcacctgaatctacccctttcctttccaattgagcaagatgctgataaatgattcacaatggacatgtggacagaataaaaatctttggatattatatggtactgtgtatttcaaggttcaagattactctctacaatgtgtgaatttttgtttcagatgacttaattcttgttcattcattatatatatatatatatatata

SEQ ID NO: 68 - protein, 241 aa, Sapium sebiferum L.
MADGNVNSQEQMAKQEEQRLKYLEFVQVAAIHAVVTFTNLYVYAKNKSGPLKPGVETVEGTVKSVVGPVYGKFHDVPIEVLKFVDRKIDQSVSSLDSRVPPVVKQLSAQAFSVAREAPVAARAVASEVQTAGVKETASGLARTLYFKYEPKAKELYTKYEPKAEQCAASAWRKLNQLPVFPHVAQVVMPTAAYCSEKYNQAVLTTAEKGYRVSSYLPFVPTERIAKLFRNEAPESTPFLSN

SEQ ID NO: 69 - DNA, 1252 bp, Sapium sebiferum L.
  source 1..1252: mol_type = genomic DNA
ctacttttccctagcattagtattctaggccccactctgtagattcctccagctgcctgatctaattttttatcaactcttgaccgttcgatcatcccaacggctcagattcactagtacttttctcacaccgtatctccgattctccatgactccatcgatataaatcgcagtgctcatcaactgaattctcgaaattgcggttacaagctgctataagaagcgaaaagaaacgctgagaaacaggatccgttcctcctccctcgttttttactccttacaagatggagaccgagaagaagattcctgaattgaagcacttagggttcgtgaggatggctgctattcagtcactgatttgcgtctcgaatctctacgattacgcgaagcataactcaggacctttgagatccactgttggaaccgtggagggtgccgtaaccaccgtagtaggtccagtttaccagaaattcaaagaccttcctgatgatcttcttgtatatgttgataagaaggtggatgaaggaacacacaagtttgataagcatgctccacctattgctaagaaggctgcgagccaagcccatagtttgtttcatatagccttggagaaggtcgaaaaactcgtgcaggaggctcgtgcaggaggacctcgtgctgctctgcattttgtggctacagagtcgaagcacttggcgttgacccaatctgtgaagctgtatagtaaacttaatcagttccctgtcattcacactgttacagatgtaacccttcccacagctactcactggtcagataagtataaccatacccttatggacctgacccggaagggttatacgatctttggttatttgcctttggttcctattgatgacatatctaagacatttaaacaaagtaaagcagaggagaaagaaaatgcaactacgcataaatctgattcatcggattccgactaaacggttgccatcatgtctaatgggtgtggtttgttaagtatagtggtttgcgaaaatgttctagggtttatgagcctgctcgaaagatgctgagaaatggaaatctgtactatttaggagtttttccgtactataataatgagtatgaatggtttgtaaattctgccttgtgctttctcgacaagtatatcatgcttctattttttactactacttactggactactgaattgtctcataattgtccctagtgtctaattaaatatcacctccaaaatattattgaaaaa

SEQ ID NO: 70 - protein, 225 aa, Sapium sebiferum L.
METEKKIPELKHLGFVRMAAIQSLICVSNLYDYAKHNSGPLRSTVGTVEGAVTTVVGPVYQKFKDLPDDLLVYVDKKVDEGTHKFDKHAPPIAKKAASQAHSLFHIALEKVEKLVQEARAGGPRAALHFVATESKHLALTQSVKLYSKLNQFPVIHTVTDVTLPTATHWSDKYNHTLMDLTRKGYTIFGYLPLVPIDDISKTFKQSKAEEKENATTHKSDSSDSD

SEQ ID NO: 71 - DNA, 938 bp, Sapium sebiferum L.
  source 1..938: mol_type = genomic DNA
gagtattcacactctggcctgattgggtttgctataaagggcgatcgttgcaacgctccatattgtctacttggttttgtttcaaatctcatcattttgtaaatttgcgacagtgtagcgttttctaggaaaaaggttgctaaaggaaagtagttatcaaaccgcagaaatggcggaatccgaacttaatcaacacacagatatggttcaagatgatgataaaaaactcaagtatctagattttgtacaagtggccgcgatctatgttgtggtttgtttctctagtatctatgaatatgctaaggaaaactccggtccactaaaaccaggggtccaagccgttgagtgtaccgtcaaaactgtaataagtccggtttacgagaagtttcgcgacgtaccttttgaactccttaaattcgtcgatcgtaaagttgacaactctctaggcgagttggacaggcacgtgccgtcgctggtgaagcaggcatcaagccaagctcgagctgtggctagtgaaattcaacatgctggattggtagacgcaactaagaacattgcgaagacgatgtatacaaagtatgaactgacggcttggcagctctactgcaaatacaagccggtggctaagcgttacgcggtgtcgacctggcgctcattgaaccagcttcctctgtttcctcaagcggctcagattgcaatcccaactgctgcttcgtggtctgagaaatacaataagatggttcgttacacgaaagatagaggatatccagcggcggtgtatctgccattgatctcggttgagaggattgccaaggtgttcaatgaagacttaaacgggcccaccgtccctaccaatggatcatccgccgcagcacaatagttttcattttatgtatttatgtcagattgaagacgctccggagattttgaaaacctga

SEQ ID NO: 72 - protein, 194 aa, Sapium sebiferum L.
MAESELNQHTDMVQDDDKKLKYLDFVQVAAIYVVVCFSSIYEYAKENSGPLKPGVQAVECTVKTVISPVYEKFRDVPFELLKFVDRKVDNSLGELDRHVPSLVKQASSQARAVASEIQHAGLVDATKNIAKTMYTKYELTAWQLYCKYKPVAKRYAVSTWRSLNQLPLFPQAAQIAIPTAASWSEKYNKMVRYT

SEQ ID NO: 73 - DNA, 2526 bp, Sorghum bicolor
  source 1..2526: mol_type = genomic DNA
atggacgagtccggggaagcgagcgtcggctccttcaggatcggcccgtcgacgctgctgggccgcggggtggcgctccgcgtgcttctcttcagctcgctgtggcgcctgcgggcgcgcgcgtacgccgccatctcgcgcgtgcgcagcgcggtgctgccggtggcggcgtcctggcttcacctcaggaacacccacggcgtcctcctcatggtcgtcctcttcgccctctccctgaggaagctctccggcgcgcggtcgcgggcggcgctcgcgcgccggcgcaggcagtacgagaaggccatgctgcatgccgggacgtacgaggtctgggcccgcgccgccaatgtgctcgacaagatgtctgatcaggtccatgaggcggatttctatgacgaggagctgatcaggaacaggcttgaggacctccggaggcggagggaggacgggtcgctgcgggacgtggtgttctgtatgcgcggcgatcttgttaggaacttggggaacatgtgcaatcctgaacttcacaagggcaggctagaggttcctaagcttataaaggaatacattgaagaggtttctattcaactaagaatggtgtgcgaatctgacactgatgagttgctattgggagagaagcttgcctttgttcaggagaccaggcatgcctttgggaggacagccctactcttaagtgggggtgcttcactggggtctttccatgtaggtgtagtgaaaacattggttgagcataagcttctgcctcggattatagcaggatcaagcgttggttccattatatgttcgattgttgctacccggacatggcctgagattgagagcttcttcacagactcattacagaccttgcagttctttgataggatgggtggaatttttgcagtgatgaggcgagtcaccactcatggtgcactgcatgacattagccagatgcaaaggcttctgagggatctcacaagtaacttaacatttcaagaggcttatgacatgactggccgtgtccttgggatcaccgtttgctctcctagaaaaaatgagccaccccgctgcctcaactatctgacgtcgccgcacgttgttatttggagtgctgtaactgcctcttgtgcatttcctgggctctttgaagctcaggaactgatggcgaaggatagattcggcaacatagttcccttccatgcaccctttgccacagatcctgaacaaggtcctggagcatcaaagcgccggtggagagatgggagcctggaaatggatttgcccatgatgagactcaaggagttgtttaatgtaaaccatttcattgtgagccaaactaatcctcacatttctcccctcctccgaatgaaagagcttgttagagtctatggagggcgctttgctggaaagcttgctcgtcttgctgagatggaggttaagtatcgatgtaaccaaatcctagagattggtcttccaatgggaggacttgcaaaattgtttgctcaggactgggagggtgatgtcaccatggttatgccggcaacagtagctcagtatttgaagattattcagaatccaacatatgcggagctccaaatggctgccaaccaaggccgcaggtgtacatgggagaagctctctgcaatcagagcaaactgtgccatcgaacttgcattggatgaatctatagcagttttaaaccacaaacggaggctaaaaagaagcatggagaggacagaggctgctttgcagggtcattctaactatgttcgactcaaaactccaaggagggtaccatcatggagctgcatcagtcgagagaattcttcagaatctctctcggaagagatttcagcagttgctacttcaaccgcgcagcaaggtgctgctcttgttgtcggcacagccactctttctcaccatgttcgacgcaattctcatgacggaagtgagagtgaatcagaaaccattgaccttaattcctggaccaggagtggtgggcctctaatgaggacagcatctgctgacatgttcatcagtttcatccataaccttgagattgacacggaattaagtaggccctgtactgtggagggtggtactgcaggtatttcgtcagaatctaccttcccaaatgatccacaaccgaacaatggctcaagtgttactactccaggtagatgcacagaaaattctgagaccgaggcatacgacactgtcaacaccagagccagtcaggcttctactcccacaagcatcgctgtttctgaaggagatttgctgcagcctgaaagcattgctgacggtatcctgcttaacattgtgaaaagagatgccttgcaggctcaaaatgacagcgtaactgaattggccgaaagctcctgcactgaaacatatgcggaaacttgtgacaccatctcagggtctggcactgctgaagataacaaggatactgctgactcaagcaatcactcacttgatattgatgcttttgtagtttcgcatcaaccttcagctgatgattag

SEQ ID NO: 74 - DNA, 3099 bp, Triticum aestivum
  source 1..3099: mol_type = genomic DNA
atgcccgcgcctgcaggtgcgtgcagccaagccccaccgctcgccttctattccgcgtcccctagcttggcccggccctgctccgatccaaggccgcggcggtggcccagtgccctctccctcctgccacgccgtccgccgcccatggacgtcatcaccaacgaggcgcgcgtgggggcgttcgcgatcggcccgtccacggcggcggggcgcgcgctcgcgctgcgcgtgctcctctgcggctcgctggcgcggctgcggcaccgcctcgccgccgcgctgcgcgccgcggcgccgctggcggcggcctggctgcacccgcgccacaacacgcgggggatcctgctcgccgtctgcgccgtcgcgctgctgctgcgcggccgcgggggccgcgccggggtgcgcgcgcgggtgcagtccgcctaccgccgcaagttctggcggaacatgatgcgcgccgcgctcacctacgaggagtgggcgcacgccgcgcggatgctcgagcgggagacgccgcgccgcgtcaccgacgccgacctctacgacgaggagctcgtgtgcaacaagctccgtgagctcaggcaccgccgtcaggagggctcgctcagggacatcgtcttctgcatgcgcgccgatctgctcaggaaccttggtaacatgtgcaaccccgagctccacaagttgaggctgcaggtgcctaaaaccatcaaggagtacattgaggaggtatctactcaactgaaaatggtttgcaattctgattcggacgagttaccccttgaagagaaactggcatttatgcatgagacaagacatgcctttggtagatcggccctactgctaagtggaggtgcttcatttggctctttccatgtgggtgttgtgaaaaccttggtagagcataagcttctacctaggattatttcaggatcaagcgttggcgcaataatgtgtgctattgtagccacacggtcatggccagaactagagagtttttttgaggagtggcattccttgaaattctttgaccagatgggtgggatctttcctgtatttaaaagaattttgacgcatggagcggttcatgacattaggcacttgcagacgcagttgagaaatcttacaagcaatttgacatttcaagaggcatatgacatgactggccgggttctcgttgttactgtgtgttctccaagaaaacatgagccaccacgatgcctgaactatttgacatcacctcatgttctcatttggagtgcagtaactgcttcctgtgcttttcctggactttttgaggcccaggagttgatggccaaagatagattcggagaaacagttccttttcatgctccattcttgttgggtgtggaggaacgagctgacgctgctacacggcgctggagagatggcagcttagaaagtgatttacccatgaagcaattgaaggaattattcaacgtaaatcacttcatagtaagccaagccaatcctcacattgctccattactgagactaaaggagatcatcagggcttacggaggcagctttgctgcaaagcttgctgaacttgctgagatggaagttaagcataggttcaatcaagttctggaacttggatttccattaggaggaatagctaagttgtttgctcaacattgggaaggtgatgtgacaatcgttatgccagccacacttgctcagtattcgaagatcatacagaatccttcgtattctgagcttcagaaagccgcaagtcagggtaggcgatgcacttgggaaaagctctctgctatcagggcaaactgcgctattgagcttgcattagatgaatgtgttgccctcctgaaccacatgcgtaggctgaagagaagtgcagaaagagcagctgcttcacaaggatatggtgctacaattagactctgtccatctagaaggattccatcatggaatctcatagcaagagaaaattcaactggttctctcgatgaggaaatgctcacatgtcccactgttacgagccatcaagcagttggagggactgctgggccatctaacagaaatcaccatctccaacatagtatgcatgatagcagtgacagtgaatctgagagtatagacttgaactcatggacgagaagtggtggccctctcatgagaacagcctcagctaataaattcatcagctttgttcagaaccttgagattgacacagaattcagaacaatttcaccaagggggagcgaaggtgatattgttacaccgaatagtaacttatttgctggtcacccaattggtagagagccagttgataaccatccagggcctgctactcctggtaggacctcaggcaattcaggttgcgatcctcatgatactcctgttcctaggtctccatttggtctttccacaagtatcatggtccctgaaggtgacttgctgcagccggaaaagattgagaatggtattttattcaatgttgtgagaagggatgctcttgtagcgactactagcggagttgaacctcatggatcttcacaggaagcagatgtggaaactgtaccgaccgagtgcctttatggtgcttcggatgacgacgacgacaacgtggaactgaatgctgatcatgaagcattatctgaccctggagatcagagatcctcagttgcaggaaacctagatccgtccacttccatggattgtcaagctgatgaaacaagtactactcgatcagaagctccatctctctttaatatctgtgtggagattcctccagcaaccatgatcagagaaaatagtcggcccgacgagccttcttcagacataagactggagattgtaaagacagaatgccctgatgagaattcagctgctgggaacgatgaagttggctcagttcctgccaataaagaatcttcctattgttctcagacagctgaaaatagacagcagcatcaagttgatatgggatctgtgaactcctgtagtgtttcagtttcagaagatgataggcatgtcagcctcatttcgaacgagaaaccagttactacttccagtggcggagcggagagtatgacatctggaagaaatgaagctgactag

SEQ ID NO: 75 - DNA, 2198 bp, synthetic construct
  misc_feature 1..2198: note = S. bicolor SDP1 hpRNAi fragment
  source 1..2198: mol_type = other DNA
gcggcggcgtggctgcacccgcgcgacaacacgcgcgggatcctgctcgccgtctgcgccgtcgcgctgggtgcagtccgcctaccgccgcaagttctggcggaacatgatgcgcgccgcgctcacctacgaggagtgggcgcacgcggcgcggatgcttggagtgcagtaacagcttcctgtgcttttcctggactttttgaggcccaccatctaggaggattccatcctggaatctcatagcaagagaaaattcaactggttctctatgtgcaatcctgaacttcacaagggcaggctagaggttcctaagcttataaaggaatacattgaagaggtttctattcaactaagaatggtgtgcgaatctgacactgatgagttgctattgggagagaagcttgcctttgttcaggagaccaggcatgcctttgggaggacagccctactcttaagtgggggtgcttcactggggtctttccatgtaggtgtagtgaaaacattggttgagcataagcttctgcctcggattatagcaggatcaagaagggtggacccagctttcttgtacaaagtggtctcgaggaattcggtaccccagcttggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggaagctgggttcgaaatcgataagcttgcgctgcagttatcatcatcatcatagacacacgaaataaagtaatcagattatcagttaaagctatgtaatatttgcgccataaccaatcaattaaaaaatagatcagtttaaagaaagatcaaagctcaaaaaaataaaaagagaaaagggtcctaaccaagaaaatgaaggagaaaaactagaaatttacctgcacaagcttggatcctctagaccactttgtacaagaaagctgggtccacccttcttgatcctgctataatccgaggcagaagcttatgctcaaccaatgttttcactacacctacatggaaagaccccagtgaagcacccccacttaagagtagggctgtcctcccaaaggcatgcctggtctcctgaacaaaggcaagcttctctcccaatagcaactcatcagtgtcagattcgcacaccattcttagttgaatagaaacctcttcaatgtattcctttataagcttaggaacctctagcctgcccttgtgaagttcaggattgcacatagagaaccagttgaattttctcttgctatgagattccaggatggaatcctcctagatggtgggcctcaaaaagtccaggaaaagcacaggaagctgttactgcactccaagcatccgcgccgcgtgcgcccactcctcgtaggtgagcgcggcgcgcatcatgttccgccagaacttgcggcggtaggcggactgcacccagcgcgacggcgcagacggcgagcaggatcccgcgcgtgttgtcgcgcgggtgcagccacgccgccgc

SEQ ID NO: 76 - DNA, 22 bp, synthetic construct
  misc_feature 1..22: note = Oligonucleotide primer
  source 1..22: mol_type = other DNA
ttttaacgatatccgctaaagg

SEQ ID NO: 77 - DNA, 23 bp, synthetic construct
  misc_feature 1..23: note = Oligonucleotide primer
  source 1..23: mol_type = other DNA
aatgaatgaacaagaattaagtc

SEQ ID NO: 78 - DNA, 22 bp, synthetic construct
  misc_feature 1..22: note = Oligonucleotide primer
  source 1..22: mol_type = other DNA
cttttctcacaccgtatctccg

SEQ ID NO: 79 - DNA, 25 bp, synthetic construct
  misc_feature 1..25: note = Oligonucleotide primer
  source 1..25: mol_type = other DNA
agcatgatatacttgtcgagaaagc

SEQ ID NO: 80 - DNA, 18 bp, synthetic construct
  misc_feature 1..18: note = Oligonucleotide primer
  source 1..18: mol_type = other DNA
gcgacagtgtagcgtttt

SEQ ID NO: 81 - DNA, 25 bp, synthetic construct
  misc_feature 1..25: note = Oligonucleotide primer
  source 1..25: mol_type = other DNA
atacataaaatgaaaactattgtgc

SEQ ID NO: 82 - DNA, 2631 bp, Saccharum hybrid
  source 1..2631: mol_type = genomic DNA
ctgcgacagctagaggcgccaccgcgtcctagcttcctccaacttctcgtcggagatcccttcagggatgcccaatgccaccgcccctaagtcaacctgcgggagctggagcttcgccagggtcagagctgcggcagcaccctggtagaccgcattcctgatgacccgcggggtgcgctccatgaagaagtgcattcgcccaaccaagtcgagtgggtcgcctggagggggcggggaagcaaaacgttgcatgcacctagcgccctggcagcgagctcctgtagtatcacctgcgtcgcctccagctcatgctcgcaagcctccagggcggcccggcagtgctccaacactttcgcctcctcctacagctccttccacatgcagtcgtgctccgcacgcaccttctccacctttttactcttttctttctcttttcttggcccatctttggtattttcacaaatgtccccctacaaatgataaatcaccaaaactcatggagcttgctagttataaactctaattctaagtttggtgtttatttgagtggattttgtgtgaaagttggtggttagaaataggagttaaggaccgccaacaagatcccccacacttagccctttgctcatcctcgagtaaagttcaaggactaaggtggaacatctcctcaaatggtacgatgcctgcatataagttattccaagcctcacctatacatgtgaactttgaagtgtctaccacgccatcttgggtggttgagaaatggaacagatcagaatccagtcatctttacctctcttgcttagataacttgggtttttgtaaggttttcaaatttaaaacatagtcttgctcctcaaatgattctctcatatagctcaatgtgtatggtttctcaccaaggcaatgttttgcctcttttcatcctacttctaatatttcttttgtggagcttagggtagggaatgaaaaggaagcatacttgcattgcatatgttactaagtcaaaaaccaaatctgaggagaagcaagtcatacaatctgatcaagatgtgcaagtgtgtggatatgtggattaagataactcctgtttattcatgctctcctccttaataaactttagagggcatggcaatctttgcatgggccttcatgagctcatcgtatgtctaagcatggagctcatcatttatataagcatggtgataccaaaattactccttttgagcatgtttatatttaggaggacgttttacctgttgaggtaaatctgaacgctaataaatcggctaagcaaaataatttatcacctgttgattctaacaatttgatgatggacaatattgatgaggtgactgacaaatgattgaaggctttaaaggagattgagaaggataaatctacaataaaaatgtaaagaagaaagcattcaaagtgtgagatctggtgtggaagactattttgcctcttgggggtaaaagacaacaagtttagtaagtggcctcaaaattgggagggcccatgcaagattgttaaagtaattgttttggattgacggaggcatttcaaggtgatcatctacctagagctctcaatgggaggtgctcgaagacatattacccatgtgtatggcaagatgtttagctagtaactgactgatagtgtaaacgatctccaatggggcaagacatattacctaaggccaggctggtttttgcaagttcgagtaggatatagagattctcgtgcgagttgtaaacgatctccaatggggcaagacatcctaccctatatatagtgaaggggcagtagctgattgagaatcaatcaatcaagcacaatataatttattaattttttattcaaacccaatttttttccttttccaaccctaattatagttttccttttgcctctaggacaaattgacgtgttccgggtatcctgctgaattaagaacaaccctaggtgcacctgtcccgatagagtcccacctgggtaggcattcatagggattcgtgtatttcctgcaaaaaagcgattaagctggcttctaaaactggctaggccggattctgtggccttcactaccaggtgattttcatgtgatccgtgcattctagcactttgctatgtaacccaaacttaagtcgacaactataaatatgctacttgcaggatgttatcacgacacaactcctaatctacggaagcctaagtttagttttgctcggagacaagcaattgtggccagtcactatagctacgtcagagggtagtgggagcagttgcgtcgttggattgaaaacaggtggatcgtatcagatattatgcattcacatggacagtaaatgtggtacagtaacttcgcaaacaataaaatctgtcacaatttattagtgcactcctctgacgtaaatgcttctacgtcagaggatttgattccgagggccgctgcacccatcactaatgacggtctttacccatcatcatggaccattgttcacatccatgctatcactgtcgtcctgtccatgcactgcagccctctataaatactggcatccctcccccgttcacagatcacacaacacaagcaagaaataaacggtagctgccataactagtaca

SEQ ID NO: 83 - DNA, 2907 bp, Saccharum hybrid
  source 1..2907: mol_type = genomic DNA
gcataggcattgtaaaagcggtatgcctcttcttcagtgcagaatttcataccaaccttaggtatcctgtcttccatagaattttctacctgagtaggttcggtctggttggatttgtagcgggtttcatgcaaaataagttagaaatcgtgcaaacttgcaatggaggttaaatttgaaatatatttgcatagacaaaacaaatatagattatgaatggtaatccaatatgacttgcattttctaactctattgctactgtgccagatgaagaatgttgatctggagaagttttgtgagaatgtgacaacaacgggaggtcatatcaagattctgggtacccgcggagaatcggcctccatgtagttagcctcgtcaggcatggggggaattggctgagatgcccccatgtagtcgtcaggcatggagagtactggctgagatgccattgttgtgtagatcgagagaaacgagaagaatgctagtctaataatacccttccgtatgctaaccaactattataattggcaccatttttcacatgctagcgccttttgcctgctttatttaattcaattgggtccgataagcatgtgaacgtgggagacggttccgtcggacggctccgttttcttgtagcgtacggcgtggacggagaaaaggtgagggcctatctctaaaggggaacgaatggatggtggacacgtgtggggagacaccgaagggacatgccgaggaggcacacaagcttcagcaggcgtctccagactctcagaagaagaagaagctcacggcacggttgcggctggttcttgctgtcgctgtctcgtggtgcacgtttctgtgatcacgctgaaatcgaccggccggcggaccaacaggaggtcagctcggccactccgtctccgagcgcatgagtgcaccgttcgtccgcggttccttttctcgtggtgccgtgcacgcctctgcgttcaccggcaccctgaaaccaatcagaacgttccctttacaggggaaagggacaagtctgataacctctctgtttccatcgtcctctaaccgcgaagagcggacgcacaagacttagagtctatttgttcgaaattttttactctcacaaaagctagcttttatagacgggcataaaagctatcatgtcgaccggcacgtttaatatttaacttataccatatgaatatcatgtcgaactatgaggatgatacttttctgaacgtgattgcgtgagttattaaattgtacttttagttgtttgagcatgaaggtctgaactatgaatttatgatgtattgtggcttgtgagctactccgctctacatttagttggtatcataaatattattatattatcatataaatttgatcaacttgagatgctttgactcttcaagattcttggaatgacttatcatttggggtagggagtaggtttctaaggccagtctcagtggggtttcatcagagtttcatggacattaaataagctgatgtgacaccgtattgatgaagagagagatgataagagtttcatgcgagtagagagagtttcatggggatgaaactcttcttcactgtttccaaaatatagatgcattggtaagagggccatgaaatctctagtgacactgacctaagatgagattgactctagcactatgtttcaaaatctgcatgcatgcatgctttgaatattgtaacctcacattaactcccctcacacatgcatgcaaacgggcggtgcacgcaaaagaattgagtgaagatgcacatgaaaaataagtaaaatgctttggcttcatcacccggcttaaatgctcgacagaaaaacacgtcggtagtcaaggttgtgcctaacaaactggggttcacatgtaaaacacgttcatgccttagaaacggcctggagggattagatacaacttcaattatatcttagggcccctccaatattgtcagctctaaactagttttatgtgtcacggtggaggagagggaggctaaaaatataatcttgagctaacgtgaagagaagagctatttttttttgctccccaatacatgatagatacaatatgagagaaaaaatatatgaataaagaacactttacatgccagccatacaatatgagatttcatctaagagccaacacctgactcgtactgttgaaggtgtcctagttggagtggtcgatcttttagttgttagtagtgtaagacctagtttagtgctcttttcttgtctaggtttatgttgtgttttggctgccaagtgttgaacaactcaaggtaaggtcccatctaattctaaaatgatgccaaataaagatagattacaaagttaaacgacggaaaaactctaaaataggatggaaagttttgtagagtaataattggtatgaagtggcgaagtcgaccacaaccaaacataaagagttaaatgcatggtaggctcttgatcttgtctggaggtgccacttaggtccacaaactctcaaattgcatttttgacaccctaatgttattcaagtgtgccacttagatctacaaactctcaaaatgcatttctgataccctagtgttgttcaagtgtgtcacttaggcaagaaaagttagataattttgataagctatgggaccaaattaatttatgtatgcatgctcgaactagttgatgatgatggaccccataatagacactagttcatgggctggtttccttgtatagtactagctagtataactttttcaagttgtagctactactttagcttatactccgcatattacaatcaaatagaattcggaagtactataaacgggagcctataaatggagacgttttgcatcatgaggctataacaacttgagcaaaaacagaagccgtgcgcc

SEQ ID NO: 84 - DNA, 1141 bp, Saccharum hybrid
  source 1..1141: mol_type = genomic DNA
actatagggcacgcgtggtcgacggcccgggctggtctggttttggcctcttttagttactaaattgccaaaaagagtgactaaaaagtgactaaactgatttagtcctctagtcaagggactaaaccagctaaaagacatccgctgcccctcattaatgcacagaaggagagagagagggagagggaggacattttggtctttatatagtagctttaatggactttagtacctagatccaaaccggtagtgactaaagtttagtccttgaactgaactttaatccagggacatggaaccaaacatgcccttaacttttttttattctaatccctcttacattcacttgtctcacaaagtggcaagtcatttgccaccctcactaccagtggcgactggttaaatatcctcatgtttggttttttttagtaaccaaatactgcaagctattgggaaaaaaggcaaaaaattatctccttgcttatagttgtataatccatgatccggcaattgtttgttacggagatcctgaatcctctgacgtagagtttaatcaattttagctcaagaataatacactataaagtggatatgacaatcaccgtagtacttatttatcttgtagtagtatactgtattcgacctgcgattatgataaaggcatcagaaactagagtactttctagaatctttagtcagtttctgtaagatgaacgtgactaggaaacttatactgttgcaatcctctgacattctctgattgaaactcggtttccaaaaatcatatgttactaaacaaaacatatctaaccaaatactatgtggtagtgtagatttatatgctgtgtactgaaagtgacgtcaagtatagtagtggcagagactcaaaagatacctgcggattctgaataccacaaccataaaaaacaggatgatgttatacttgtccccttccatgatacaggactgtttagtaatttcccaaacagcccataatacattctgcaccctttattaaacctctactagctacaacatcttactccatcttgtctagttggacaagttctctctttcttggctgactccaacttactacaccgcaacttcttgtgcccttgttccaaccatcacaattgag

SEQ ID NO: 85 - DNA, 4438 bp, Saccharum hybrid
  source 1..4438: mol_type = genomic DNA
aagtacaaacgtagactctgacatacacgcacgtagactctgacatacacgcataaacgaacgaagaatgttattatttatgttttgagtgggaatatttggtactgctatgattcacgtgtgtaaggaaggattcaagaagaaaggatgcgtttagttcgcgaaaatttttgacttttaccactatagcactttcgtttgtatttgttaattagtgtccaatcatggactaattagactcaaaagatccgtctcgtggttttaaaccaaactgtgtaattaattttttttatctatatttaatgctccatatatgtgtcaaatattcgatataacgaagaatcttgaaaatttttaggaactaaacatggccaaagtgttgtcccgactgagaaactttggaagcagaataaaggctcaaaggaacatttaaaagaagaggatgatatataatcaaaagtggcgacaaagaagtgtgtacgacccactcgagattgacgaaggacagcttctttgttcttttgtgtgttactgaatatgtaataatcttgtatagattggtttttaaaatacggtggcaaattaaagacgatatcacttacaaagacatggacaatgtggaggggccaaaagttatataaacgacacgccgaatcggtgatgaacaccacatgcctcccataaagacggtgtatcaatctttgatataatgggtatccgtttgaggcggcatttatacttgatctagtgaaattacaaggagaggaaaagaagtttaagagaatgataatgataatgaaaaaaatcggaggaaaaagaacatgaacaaagcaagaggagatagccgtgcacacaaaatagagataatttcctcttagaactatgaaaacttcctcttctttctgcaacactgatttgagtttttgttctctatctagcatttcagtccatcttgatgtcaagtgacatgtaaaaagacgtattgcccccattgctgttttaaattgtctccacacttgacaacaatttaatgagttgttaaaatattatgtgtgtttatggccaattatactttttagttttgagtttttcatgaagtcattaagatgctaaaaataatataaagttgtcaatgcttgtcggaagccccaatatgtgactaaaatgctgctaaaagtttatagcatttttttaaaaatctaaacaaattgaaaaaagaaatccaaactagaaattgtagatcttatcgaaaactataagttttatataaaaggcgactttatctaacaccacacaagaaagatgtgctttttctaagaagacaagtcttagtatgtgattaatatgctactgaaaatttatattatttttaagcattttaataacctcaaatggaaacatacaaaactaagttgcagatcttatcaagagctataatttttatataaaatgtatatttaaataacaccatacaagaaagatatatgattttttctaagacgacaaagctttgtatgcaatttaatatgttgctaaaaaatcatattattttttttatcatcttaacgtcctcaaataaaaaaaaatcagactagttggtatagacctcatcgaggctacaatttttataaaaactcaacttcatccggtgttgtataaaaatgatataatttttcctagatagagcgttgccataagtgtattttggtcaagaaatatatgtatacttattaatgaaatcctaacaaaatatactttaaaatctgacggaaatgttggataggaaacaaaagcttaaatcaatgctaaatagggaagttttcatcatagttataatgagtgatttctccacaaaatatgatgtaccacatgttaaatattactcgcgcacaaataatcagagcatattactttcatagcgtggtcgtggccatggcctagacttggttgtggacgtctcacttcaccaattgatagaaaaaaaacatttataagaaagaaaagatacagaaaccatcacacgcgacaacatgacttgccgaaacacaaaaccaaaacccaaactcgagaagatgctttcgagaaaaagcctgaaaagaaaaaaaatttgcacgtaaaatcaaattcggacggcgaagagggcaaacgagacagacaactgggtccacttgctgataaaaaagagagagaggagggcccacttgccggcgggcacccctcagactgtctccaacaatactgacgcaaacagaagacgcattggatgcaatgcgttgcgctgtggcaaaaaattaggtacctatttctagtgtattccaacagagaacgcaaaagaagatgccgtactgcgccatgcattcatgtgggaccggggaggatgcgggcaacagcagtttgcacgacccattggccggagcatgcgacgtatatttgcgttgcgcctcgcttcctacgcaaaatgtgtcgttggtatgcctaccctgttggagggcgttttcttctgctaaagtaacgtggagcacgcatttgcgtaggctgttggagatagtctcaccacgcggtgaccggaccaggccaattcccgagcccaaaaagaaaaaagcacacacacagagacacacgctctcgctctcgcctccctgacgctggatttaagcagagcagggagcagaggtgcaaccgcccaccacgatctcccctcccgcacgccccgcgggcagacccagccaaggcaaggcagccgcgaaccggagcacgccggccggtgtcgcctcccgcgccggcggcctgctgctcgctcgccctcgcttccgcattggatcacgcggcggttggcgacttggtggtgtctgctgctggtgattgcgcctagccggccgacgcggagacggtgaggcgctgctcttcgcttctctccccactgctcccctcagcggtttctctctccctgttatgcgtggaggagccctgcccccgcggaacggaagcctccgccggatctctgttacgccgcggttactgcctcgccctggatttgaacttgtttcgtaattttcccttgctgcgcttctcgatttcggggaggggttctgccggcagctctgccgctccacctgacttggggacctttctatgttccgcgacagcagcattgatgatctgcttgtctcttgagtttttttttcgtgcgatgcatcgagcgcgtggggacacgatcacgcctgatgggcggtagtccgcgatccgcatttctgaatcccggcgcctagccgaggtgcctcggtgcttcctggttgccttgctgctattcccttcttcggatccgctctcgtacggctggcacggtggttgcggccttagaatttcgtggcggcggtttggttggattggtgatgctgctccgtccgcatttatgaaggaatgttctccaaacttttaagctgctcgtgtactcggagtattgaattgcctgttccttgccgctataggaggccctgggccagcctaccccgctttgggttgtgattggtgatttccggcagctgttattgtttcatgattcgtgtggggaaaaaaagtttttttggttcacgagtggtttctggtgcatgttttgacaagttttctatgatgctggtactgtctttacccctgctagagtagtttggtggtgcgttttcctattaggtgggaatttaatcactttcccactttatcgtatctctactatggtaaccatcttttggcaattttgattggtatagtcatgtttaagataagcttttgaattcaatgatcttgccgttcattagctagcacttaattttgtagagctgcttggatcaccaaagtgccgctcaatcttgttcaagtgcctatgatatatgggattctgatggaactcttagcagtcgtgtccttaggcagtcggcaccttgataaggttccaagagttcaatcttacggaagaaatagtgagcttgatctgagttcagatcggttgtcttcacacttcacgattaattaccacgtttttaaggtgtgcattctcacttctttacttccatcgtcaatcttcttaactggttgggttggaggtgtggtcatgcacccaaccacataggttgagtcctcttcaactcgaatttaggtgcctatttttttcttaataaaaaaggccacctgattctccttggttggtcacatttttttcttaataaaaaaaggccacctcaatgtttctccttttagcttgagcactttttctggatctcctctttcttcttaattctgatccaagtgtcatcagcgttatatttatttgaacctgcttgcttttgtaagcctgatcagtttgcaaaagttactagaacaatttaaccatctgtgcttgttatttctgcaggcatcaagtttctaacaatttgaagtacctaaa

SEQ ID NO: 86 - protein, 145 aa, Sesamum indicum
MAEHYGQQQQTRAPHLQLQPRAQRVVKAATAVTAGGSLLVLSGLTLAGTVIALTIATPLLVIFSPVLVPAVITIFLLGAGFLASGGFGVAALSVLSWIYRYLTGKHPPGADQLESAKTKLASKAREMKDRAEQFSQQPVAGSQTS

SEQ ID NO: 87 - protein, 382 aa, Cinnamomum camphora
MATTSLASAFCSMKAVMLARDGRGMKPRSSDLQLRAGNAQTSLKMINGTKFSYTESLKKLPDWSMLFAVITTIFSAAEKQWTNLEWKPKPNPPQLLDDHFGPHGLVFRRTFAIRSYEVGPDRSTSIVAVMNHLQEAALNHAKSVGILGDGFGTTLEMSKRDLIWVVKRTHVAVERYPAWGDTVEVECWVGASGNNGRRHDFLVRDCKTGEILTRCTSLSVMMNTRTRRLSKIPEEVRGEIGPAFIDNVAVKDEEIKKPQKLNDSTADYIQGGLTPRWNDLDINQHVNNIKYVDWILETVPDSIFESHHISSFTIEYRRECTMDSVLQSLTTVSGGSSEAGLVCEHLLQLEGGSEVLRAKTEWRPKLTDSFRGISVIPAESSV

SEQ ID NO: 88 - protein, 417 aa, Cocos nucifera
MVASVAASAFFPTPSFSSTASAKASKTIGEGSESLDVRGIVAKPTSSSAAMQGKVKAQAVPKINGTKVGLKTESQKAEEDAAPSSAPRTFYNQLPDWSVLLAAVTTIFLAAEKQWTLLDWKPRRPDMLTDAFSLGKIVQDGLIFRQNFSIRSYEIGADRTASIETLMNHLQETALNHVRNAGLLGDGFGATPEMSKRNLIWVVTKMQVLVEHYPSWGDVVEVDTWVGASGKNGMRRDWHVRDYRTGQTILRATSVWVMMNKHTRKLSKMPEEVRAEIGPYFVEHAAIVDEDSRKLPKLDDDTADYIKWGLTPRWSDLDVNQHVNNVKYIGWILESAPISILENHELASMTLEYRRECGRDSVLQSLTAISNDCTGGLPEASIECQHLLQLECGAEIVRGRTQWRPRRASGPTSAGSA

SEQ ID NO: 89 - protein, 423 aa, Cocos nucifera
MVASIAASAFFPTPSSSSSAASAKASKTIGEGPGSLDVRGIVAKPTSSSAAMQEKVKVQPVPKINGAKVGLKVETQKADEESSPSSAPRTFYNQLPDWSVLLAAVTTIFLAAEKQWTLLDWKPRRPDMLADAFGLGKIVQDGLVFKQNFSIRSYEIGADRTASIETLMNHLQETALNHVKSAGLMGDGFGATPEMSKRNLIWVVTKMRVLIERYPSWGDVVEVDTWVGPTGKNGMRRDWHVRDHRSGQTILRATSVWVMMNKNTRKLSKVPEEVRAEIGPYFVERAAIVDEDSRKLPKLDEDTTDYIKKGLTPRWGDLDVNQHVNNVKYIGWILESAPISILENHELASMSLEYRRECGRDSVLQSLTAVSNDLTDGLVESGIECQHLLQLECGTELVKGRTEWRPKHSPALGNMGPTPGGSA

SEQ ID NO: 90 - protein, 414 aa, Cocos nucifera
MVASVAASSSFFPVPSSSSSASAKASRGIPDGLDVRGIVAKPASSSGWMQAKASARAIPKIDDTKVGLRTDVEEDAASTARRTSYNQLPDWSMLLAAIRTIFSAAEKQWTLLDSKKRGADAVADASGVGKMVKNGLVYRQNFSIRSYEIGVDKRASVEALMNHFQETSLNHCKCIGLMHGGFGCTPEMTRRNLIWVVAKMLVHVERYPWWGDVVQINTWISSSGKNGMGRDWHVHDCQTGLPIMRGTSVWVMMDKHTRRLSKLPEEVRAEITPFFSERDAVLDDNGRKLPKFDDDSAAHVRRGLTPRWHDFDVNQHVNNVKYVGWILESVPVWMLDGYEVATMSLEYRRECRMDSVVQSLTAVSSDHADGSPIVCQHLLRLEDGTEIVRGQTEWRPKQQARDLGNMGLHPTESK

SEQ ID NO: 91 - protein, 419 aa, Cuphea lanceolata
MVAAAATSAFFPVPAPGTSPKPGKSGNWPSSLSPTFKPKSIPNAGFQVKANASAHPKANGSAVNLKSGSLNTQEDTSSSPPPRAFLNQLPDWSMLLTAITTVFVAAEKQWTMLDRKSKRPDMLVDSVGLKSIVRDGLVSRQSFLIRSYEIGADRTASIETLMNHLQETSINHCKSLGLLNDGFGRTPGMCKNDLIWVLTKMQIMVNRYPTWGDTVEINTWFSQSGKIGMASDWLISDCNTGEILIRATSVWAMMNQKTRRFSRLPYEVRQELTPHFVDSPHVIEDNDQKLHKFDVKTGDSIRKGLTPRWNDLDVNQHVSNVKYIGWILESMPIEVLETQELCSLTVEYRRECGMDSVLESVTAVDPSENGGRSQYKHLLRLEDGTDIVKSRTEWRPKNAGTNGAISTSTAKTSNGNSAS

SEQ ID NO: 92 - protein, 419 aa, Cuphea viscosissima
MVAAAATSAFFPVPAPGTSPKPGKSGNWPSSLSPTFKPKSIPNGGFQVKANASAHPKANGSAVNLKSGSLNTQEDTSSSPPPRAFLNQLPDWSMLLTAITTVFVAAEKQWTMLDRKSKRPDMLVDSVGLKSIVRDGLVSRHSFSIRSYEIGADRTASIETLMNHLQETTINHCKSLGLHNDGFGRTPGMCKNDLIWVLTKMQIMVNRYPTWGDTVEINTWFSQSGKIGMASDWLISDCNTGEILIRATSVWAMMNQKTRRFSRLPYEVRQELTPHFVDSPHVIEDNDQKLRKFDVKTGDSIRKGLTPRWNDLDVNQHVSNVKYIGWILESMPIEVLETQELCSLTVEYRRECGMDSVLESVTAVDPSENGGRSQYKHLLRLEDGTDIVKSRTEWRPKNAGTNGAISTSTAKTSNGNSVS

SEQ ID NO: 93 - protein, 382 aa, Umbellularia californica
MATTSLASAFCSMKAVMLARDGRGMKPRSSDLQLRAGNAPTSLKMINGTKFSYTESLKRLPDWSMLFAVITTIFSAAEKQWTNLEWKPKPKLPQLLDDHFGLHGLVFRRTFAIRSYEVGPDRSTSILAVMNHMQEATLNHAKSVGILGDGFGTTLEMSKRDLMWVVRRTHVAVERYPTWGDTVEVECWIGASGNNGMRRDFLVRDCKTGEILTRCTSLSVLMNTRTRRLSTIPDEVRGEIGPAFIDNVAVKDDEIKKLQKLNDSTADYIQGGLTPRWNDLDVNQHVNNLKYVAWVFETVPDSIFESHHISSFTLEYRRECTRDSVLRSLTTVSGGSSEAGLVCDHLLQLEGGSEVLRARTEWRPKLTDSFRGISVIPAEPRV

SEQ ID NO: 94 - protein, 308 aa, Cocos nucifera
MDASGASSFLRGRCLESCFKASFGMSQPKDAAGQPSRRPADADDFVDDDRWITVILSVVRIAACFLSMMVTTIVWNMIMLILLPWPYARIRQGNLYGHVTGRMLMWILGNPITIEGSEFSNTRAIYICNHASLVDIFLIMWLIPKGTVTIAKKEIIWYPLFGQLYVLANHQRIDRSNPSAAIESIKEVARAVVKKNLSLIIFPEGTRSKTGRLLPFKKGFIHIALQTRLPIVPMVLTGTHLAWRKNSLRVRPAPITVKYFSPIKTDDWEEEKINHYVEMIHALYVDHLPESQKPLVSKGRDASGRSNS

SEQ ID NO: 95 - protein, 356 aa, Arabidopsis thaliana
MDVASARSISSHPSYYGKPICSSQSSLIRISRDKVCCFGRISNGMTSFTTSLHAVPSEKFMGETRRTGIQWSNRSLRHDPYRFLDKKSPRSSQLARDITVRADLSGAATPDSSFPEPEIKLSSRLRGIFFCVVAGISATFLIVLMIIGHPFVLLFDPYRRKFHHFIAKLWASISIYPFYKINIEGLENLPSSDTPAVYVSNHQSFLDIYTLLSLGKSFKFISKTGIFVIPIIGWAMSMMGVVPLKRMDPRSQVDCLKRCMELLKKGASVFFFPEGTRSKDGRLGSFKKGAFTVAAKTGVAVVPITLMGTGKIMPTGSEGILNHGNVRVIIHKPIHGSKADVLCNEARSKIAESMDL

SEQ ID NO: 96 - DNA, 1539 bp, synthetic construct
  misc_feature 1..1539: note = Codon optimised sequence
  source 1..1539: mol_type = other DNA
atggctgtgtccaagaacccagagactctcgctccagatcaagagccatccaaagagtctgatcttaggcgtaggccagcttcctctccatcttctactgctgcttctccagctgtgccagattcctcatctaggacttccagttccatcactggctcttggactactgctctcgatggtgattctggtgctggtgctgttaggatcggagatccaaaggataggatcggcgaggctaacgatatcggcgaaaagaaaaaggcttgctccggtgaggttccagtgggatttgttgatagaccatctgctccagtgcacgtgagagttgttgagtctcctctctcctccgatacaatcttccagcagtctcacgctggactcctcaatctttgcgtggtggtgcttatcgctgtgaactccaggctcattatcgagaaccttatgaagtacggcctcctcatcggctccggatttttcttctcatctcgtttgctcagggattggcctctccttatctgctctcttactctcccagtgttcccactcggatcctacatggttgagaagctcgcttacaagaagttcatctccgagccagtggtggtgtctcttcacgtgatcctcatcattgctactatcatgtaccctgtgttcgtgattctcaggtgcgattccccaatcctctccggaatcaacctcatgcttttcgtgtcctccatctgcctcaagctcgtttcttacgctcacgctaactacgatctcaggtcctcctccaactccatcgataagggaatccacaagtcccagggcgtgtccttcaagtctctcgtgtactttatcatggctccaacactctgctaccagccatcttacccaaggactacttgcattaggaagggctgggttatctgccagcttgtgaagctcgtgatcttcactggtgtgatgggcttcatcatcgagcagtacatcgatccaatcatcaagaactcccagcacccactcaagggaaacgtgttgaacgctatggaaagggtgctcaagctctccatcccaacactttacgtgtggctctgcgtgttctactgcactttccacctctggctcaatatcctcgctgagcttctttgcttcggcgatcgtgagttctacaaggattggtggaacgctaagactatcgaagagtactggcgtatgtggaacatgccagtgcacaagtggatgcttaggcacgtttacctcccatgcatccgtaacggtattccaaagggtgtggctatggtgatctccttcttcatctctgctatcttccacgagttgtgcatcggaatcccatgccacatcttcaagttctgggctttcatcggcatcatgttccaggtgccactcgttatcctcactaagtacctccagaacaagttcaagtccgctatggtgggcaacatgattttctggttctttttctcaatctacggccagccaatgtgcgtgctcctttactaccacgatgtgatgaataggaaggtgggcactgagtaa

SEQ ID NO: 97 - protein, 371 aa, Cocos nucifera
MVELRSSSSEMDLDRPNIEEYLTTDSIQESPKKLHLRDLLDISPTLTEATGAIVDDSFTRCFKSNPPEPWNWNVYLFPLWCLGVIIRYGILFPLRVAILTAGWLVFFAAFIPVHFLLTAHNKWRRKIERKLVEMICSVFVASWTGVVKYHGPRPSMRPQQVFVANHTSMIDFIILEQMTAFAVIMQKHPGWVGFIQKTILEGVGCIWFNRTESKDREVVARKLREHIHGADNNPLLIFPEGTCVNNHYTVMFKKGAFELGCAVCPVAIKYNKIFVDAFWNSKKQSFTMHLFHLMTSWAVVCDVWYLEPQYIRPGETPIEFAERVRDMISVRAGLKKVPWDGYLKYFRPSPKLTERKQQIFAESVLQRLEEK

SEQ ID NO: 98 - protein, 376 aa, Arabidopsis thaliana [sequence omitted]

SEQ ID NO: 99 - protein, 371 aa, Elaeis guineensis
MVELRSSSSEMDLDRPNIEEYLPPTPSKNPPKKLHLRDLLDISPTLTEAAGAIVDDSFTRCFKSNPPEPWNWNVYLFPLWCLGVIIRYGILFPLRVAILTAGWLVFFAAFIPVHFLLTAHNKWRRKIERKLVEMICSVFVASWTGVVKYHGPRPSMRPQQVFVANHTSMIDFIILEQMTAFAVIMQKHPGWVGFIQKTILEGVGCIWFNRTESKDREVVARKLREHIHGADNNPLLIFPEGTCVNNHYTVMFKKGAFELGCAVCPVAIKYNKIFVDAFWNSKKQSFTMHLFHLMTSWAVVCDVWYLEPQYIRPGETPIEFAERVRDMISIRAGLKKVPWDGYLKYFRPSPKLTERKQQIFAESVLQRLEEK

SEQ ID NO: 100 - protein, 371 aa, Phoenix dactylifera
MVGLRSSSSEMDLDRPNIEEYLTTDSIEESPKKLHLRDLLDISPTLTEAAGAIVDDSFTRCFKSNPPEPWNWNVYLFPLWCLGVIIRYGILFPLRVAVLTAGWLVFFAAFIPAHFLLTAHNKWRRKIERKLVEMICSVFVASWTGVVKYHGPRPSMRPQQVFVANHTSMIDFIILEQMTAFAVIMQKHPGWVGFIQKTILEGVGCIWFNRTESKDREVVARKLREHIQGADNNPLLIFPEGTCVNNHYTVMFKKGAFELGCAVCPVAIKYNKIFVDAFWNSKKQSFTMHLFHLMTSWAVVCDVWYLEPQYIRPGETPIEFAERVRDMISVRAGLRKVPWDGYLKYFRPSPKLTERKQQIFAESVLQRLEEK

SEQ ID NO: 101 - protein, 371 aa, Musa acuminata
MAGLATSSTEMDLDRPNIDEYLTVESIREAPKKLHLRDLLDISPTLKEAAGAIVDDSFTRCFKSNPSEPWNWNIYLFPLWCLGVVIRYGILFPFRVIILVAGWIVFFAAFSLVHFLLGEHNKWKREIERKLVEMICSVFVASWTAVIKYHGPRPSMRPQQVFVANHTSMIDFIILEQMTAFAVIMQKHPGWVGFIQKIIVESLGCIWFNRTEAKDREIVARKLREHIQGVDNNPLLIFPEGTCVNNHYTVMFKKGAFELGCAVCPVAIKYNKIFVDAFWNSKKQSFTMHLVQLMTSWAVVCDVWYLEPQYIRPGETPIEFAERVQDMISVRAGLKKVPWDGYLKYFRPSPKLIERKQQIFAESVLQRLEEK

SEQ ID NO: 102 - protein, 372 aa, Ananas comosus
MAEALGSSSAEMDLDRPNLEEYLPTDSIQDSPKNLHLRDLLDISPTLTEAAGAIVDDSFTRCFKSNPPEPWNWNIYLFPLWCLGVVVRYGILFPLRVAVLAIGWIVFFSAFFPVHFLLKGYPKWRRKLERKLVEMMCSVFVASWTGVVKYHGPRPSTRPHQVFVANHTSMIDFIILEQMTAFAVIMQKHPGWVGFIQKTILESVGCIWFNRTESKDRGVVGRKLREHVQGVDNNPLLIFPEGTCVNNHYTVMFKKGAFELGCAVCPIAIKYNKIFVDAFWNSKKQSFTMHLVRLMTSWAVVCDVWYLEPQYLRPGETPIEFAERVRDMISARAGLKKVPWDGYLKYFRPSPKHTERKQQIFAESILRRLERK

SEQ ID NO: 103 - protein, 370 aa, Asparagus officinalis
MAGLESSSAGIDVDPPNIEDYLTSDALHQPHKKLQLKDLLDISPTLTEAAGAIVDDSFTRCFKSNPPEPWNWNVYLFPLWCLGVVVRYGILFPLRVMTLAAGWIVFFSAFLPVHYLMKGQNKWKNNIERKLVEMICSVFVASWTGVVRYHGPRPSMRPQQVFVANHTSMIDFIILEQMAAFAVIMQKHPGWVGFLQTTILESIGSIWFNRTEAKDREVVARKLREHTEGDNNPLLIFPEGTCVNNDYTVMFKKGAFELGCAVCPVAIKYNKIFVDAFWNSKKQSFTMHLMRLMTSWAVVCDVWYLEPQYLKPGETSIEFAERVRDMISVRAGLRKVPWDGYLKYFRPSPKLTERKQQIFAESVLRRLEEK

SEQ ID NO: 104 - protein, 370 aa, Oryza brachyantha
MASSSVAGDIELDRPNLEDYLPPDSLPQESPGNLHLRDLLDISPVLTEAAGAAVDDSFTRCFKSNSPEPWNWNIYLFPLWCLGVVIRYGILFPLRGLTLLVGWIAFFAAFFSVHFLFKGQKMRSKIERKLVEMMCSVFVASWTGVIKYHGPRPSTRPHQVFVANHTSMIDFIILEQMTAFAVIMQKHPGWVGFIQKTILESVGCIWFNRNDLKDREVVAKKLRDHVQHPDNNPLLIFPEGTCVNNQYTVMFKKGAFELGCAVCPIAIKYNKIFVDAFWNSKKQSFTMHLVRLMTSWAVVCDVWYLEPQYLKEGETAIQFAERVRDMIAARAGLKKVPWDGYLKHNRPSPKHTEEKQRIFADSVLQRLEES

SEQ ID NO: 105 - protein, 370 aa, Oryza sativa
MATSSVAGDIELDRPNLEDYLPSDSLPQEFPRNLHLRDLLDISPVLTEAAGAIVDDSFTRCFKSNSPEPWNWNIYLFPLWCLGVVIRYGILFPLRGLTLLVGWLAFFAAFFPVHFLLKGQKMRSKIERKLVEMMCSVFVASWTGVIKYHGPRPSTRPHQVFVANHTSMIDFIILEQMTAFAVIMQKHPGWVGFIQKTILESVGCIWFNRNDLKDREVVAKKLRDHVQHPDSNPLLIFPEGTCVNNQYTVMFKKGAFELGCAVCPIAIKYNKIFVDAFWNSKKQSFTMHLVRLMTSWAVVCDVWYLEPQYLRDGETAIEFAERVRDMIAARAGLKKVPWDGYLKHNRPSPKHTEEKQRIFADSVLRRLEES

SEQ ID NO: 106 - protein, 363 aa, Nelumbo nucifera
MDLDRPNIEEYLPSEAIQESNEKLHLRDLLDISPTLTEAAGAIVDDSFTRCFKSNPSEPWNWNVYLFPLWCFGVVVRYGILFPVRVLVLTIGWIIFLSSFIPAHFLLRSHDKWRKKIERYLVELICSFFVASWTGVVKYHGPRPSMRPKQVFVANHTSMIDFIVLEQMTAFAVIMQKHPGWVGLLQSTILESVGCIWFNRAEAKDREIVARKLRDHIQGVDNNPLLIFPEGTCVNNHYTVMFKKGAFELGCTVCPIAIKYNKIFVDAFWNSKKQSFTMHLLHLMTSWAVVCDVWYLEPQNIRPGETPIEFAERVRDIISVRAGLKKVPWDGYLKYSRPSPKHRERKQQRFVESVLQRLEKKGK

SEQ ID NO: 107 - protein, 376 aa, Vitis vinifera
MANAPDNKLTSSSSELDLDRPNLEDYLPSGSMQEPRGKLRLRDLLDISPTLTEAAGAIVDDSFTRCFKSNPPEPWNWNVYLFPLWCLGVVIRYGILFPTRVLVLTLGWIIFLSSFIPVHFLLKGNDKLRKKLERCLVELICSFFVASWTGVVKYHGPRPSRRPQQVFVANHTSMIDFIVLEQMTAFAVIMQKHPGWVGLLQSTILESVGCIWFNRTEAKDREIVARKLRDHVQGADNNPLLIFPEGTCVNNHYTVMFKKGAFELGCTVCPIAIKYNKIFVDAFWNSKKQSFTMHLLQLMTSWAVVCDVWYLEPQTLKPGETPIEFAERVRDIISLRAGLKKVPWDGYLKYSRPSPKHREQKQQSFADSVLRRLEEK

SEQ ID NO: 108 - protein, 373 aa, Nicotiana tomentosiformis
MNMNKLKTSSSELDLDRPNLEDYLPTGSIPEPHGKLRLRDLIDISPTLTEAAGAIVDDSFTRCFKSNPPEPWNWNIYLFPLWCLGVVVRYGILFPIRVIVLTIGWIIFLSCYIPVHFLLKGHDKFRKKLERCLVELICSFFVASWTGVVKYHGPRPSIRPKQVFVANHTSMIDFIVLEQMTAFAVIMQKHPGWVGLLQSTILEGVGCIWFNRSEAKDREIVARKLRQHVEGADNNPLLIFPEGTCVNNHYTVMFKKGAFELGCTVCPVAIKYNKIFVDAFWNSRKQSFTMHLLQLMTSWAVVCDVWYLEPQNIRPGETPIEFAERVRDIISARAGLKKVPWDGYLKYSRPSPKHRERKQQSFAESVLRRLEEK

SEQ ID NO: 109 - protein, 375 aa, Jatropha curcas
MATPGKLKTSSSELDLDRPNIEDYLPSGVSIQEPRGKLRLRDLLDISPTLTEAAGAIVDDTFTRCFKSNPPEPWNWNIYLFPLWCCGVVCRYGILFPIRVLVLTIGWIIFLSCYIPVHFLLKGHDKLRKKLERCLVELICSFFVASWTGVVKYHGPRPSIRPKQVFVANHTSMIDFIILEQMTAFAVIMQKHPGWVGLLQSTILESVGCIWFNRSEAKDREIVTKKLRDHVQGADNNPLLIFPEGTCVNNHYTVMFKKGAFELGCTVCPIAIKYNKIFVDAFWNSRKQSFTTHLLQLMTSWAVVCDVWYLEPQNLKPGETPIEFAERVRDIISVRAGLKKVPWDGYLKYSRPSPKHRERKQQSFAESVLQRLEEK

SEQ ID NO: 110 - protein, 376 aa, Glycine max
MNNSGTPKSSSSELDLDRPNIEDYLPSGSTIQQEPHGKLFLHDLLNISPTLSEAAGAIVDDSFTRCFKSNPPEPWNWNVYLFPLWCFGVVIRYLILFPIRVIGLTIGWIIFLSSFIPVHFLLKGHDKLRRSIERSLVEMMCSFFVASWTGVVKYHGPRPSRRPKQVFVANHTSMIDFIILEQMTAFAVIMQKHPGWVGLLQSTILESLGCIWFNRTEAKDREIVARKLRDHVQGADNNPLLIFPEGTCVNNHYTVMFKKGAFELGCTVCPVAIKYNKIFVDAFWNSRKQSFTMHLLQLMTSWAVVCDVWYLEPQNLKPGETPIEFAERVRDIISVRAGLKKVPWDGYLKYSRPSPKHRERKQQNFAESVLRRWEEK

SEQ ID NO: 111 - protein, 371 aa, Sesamum indicum
MSKLNTSSSELDFDRPNIEDYLPSGSIQEPHGKLRLRDLLDISPTLTEAAGAIVDDSFTRCFKSNPPEPWNWNIYLFPLWCLGVVIRYGLLFPLRVIVLTIGWIIFLSCYFPVHFLLRGHDKLRKRLERGLVELICSFFVASWTGVVKYHGPRPSMRPKQVFVANHTSMIDFIVLEQMTAFAVIMQKHPGWVGLLQSTILESLGCIWFNRSESKDREIVAKKLREHVHDADNNPLLIFPEGTCVNNHYTVMFKKGAFELGCTVCPIAIKYNKIFVDAFWNSRKQSFTTHLLQLMTSWAVVCDVWYLEPQNLKPGETPIEFAERVRDIISVRAGLRKVPWDGYLKYSRPSPKHRERKQQSFAESILRRLEEK

SEQ ID NO: 112 - protein, 364 aa, Brachypodium distachyon
MASSLDAPNLDDYLPTDSLPQEPPRSLNLRDLLDISPVLTEAAGAIVDDSFTRCFKSNSPEPWNWNIYLFPLWCFGVVVRYGLLFPLRVLTLGLGWMVFFAAFFPVHFLLKGQNKLRSKIERKLVEMMCSVFVASWTGVIKYHGPRPSSRPYQVFVANHTSMIDFIILEQMTAFAVIMQKHPGWVGFIQKTILESVGCIWFNRNDLKDREVVGRKLRDHVQRPDNNPLLIFPEGTCVNNQYTVMFKKGAFELGCAVCPIAIKYNKIFVDAFWNSKKQSFTMHLGRLMTSWAVVCDVWFLEPQYLREGETSIAFTERVRDMIAARAGLKKVLWDGYLKHNRPSPKHTEEKQRIFAESVLKRLEES

SEQ ID NO: 113 - protein, 371 aa, Setaria italica
MASSSVAADMELDRPNLEDYLPPDSLPQEAPRNLHLRDLLDISPVLTEAAGAIVDDSFTRCFKSNSPEPWNWNIYLFPLWCLGVVIRYGILFPLRSLTLAIGWLAFFAAFFPVHFLLKGQDKLRSKIERKLVEMMCSVFVASWTGVIKYHGPRPSTRPHQVFVANHTSMIDFIILEQMTAFAVIMQKHPGWVGFIQKTILESVGCIWFNRNDLRDREVTARKLRDHVQQPDKNPLLIFPEGTCVNNQYTVMFKKGAFELGCAVCPIAIKYNKIFVDAFWNSKKQSFTMHLVRLMTSWAVVCDVWYLPPQYLREGETAIAFAERVRDMIAARAGLKKVPWDGYLKHNRPSPKHTEEKQRIFAESVLMRLEEK

SEQ ID NO: 114 - protein, 376 aa, Cicer arietinum
MNSTGTLKSSSSELDLDRPNIEDYLPSGAAIQQEPRGKLRLHDLLDISPTLSEAAGAIVDDSFTRCFKSNPPEPWNWNIYLFPLWCFGVVVRYLILFPTRVLGLTLGWIIFLSAFIPVHLLLKGHDKLRRNIERSLVEMMCGFFVASWTGVVKYHGPKPSRRPKQVFVANHTSMIDFIILEQMTAFAVIMQKHPGWVGLLQSTILESVGCIWFNRTEAKDREIVARKLREHVQGADNNPLLIFPEGTCVNNHYTVMFKKGAFELGCTVCPVAIKYNKIFVDAFWNSRKQSFTKHLLQLMTSWAVVCDVWYLEPQNLKPGETPIEFAERVRDIISHRAGLKKVPWDGYLKYSRPSPKHRERKQQNFAESVLRRLEEK

SEQ ID NO: 115 - protein, 371 aa, Zea mays
MASSSVAADMELDRPNLEDYLPPDSLPQEAPRNLHLRDLLDISPVLTEAAGAIVDDSFTRCFKSNSPEPWNWNIYLFPLWCFGVVIRYGLLFPLRSLTLAIGWLAFFAAFFPVHFLLKGQDKLRNKIERKLVEMMCSVFVASWTGVIKYHGPRPSTRPHQVFVANHTSMIDFIILEQMTAFAVIMQKHPGWVGFIQKTILESVGCIWFNRNDLRDREVTARKLRDHVQHPDKNPLLIFPEGTCVNNQYTVMFKKGAFELGCAVCPIAIKYNKIFVDAFWNSKKQSFTMHLVRLMTSWAVVCDVWYLEPQYLREGETAIAFAERVRDMIAARAGLKKVPWDGYLKHNRPSPKHTEEKQRIFAESVLRRLEEK

SEQ ID NO: 116 - protein, 378 aa, Gossypium hirsutum
MNSSEGKLKSSSSELDLDRPNIEDYLPSGSSIQEPHGKLRLRDLLDISPALTEAAGAIVDDSFTRCFKSNPPEPWNWNVYLFPLWCCGVVFRYLILFPMRALILTIGWIIFLSCFIPVHFLLKGNDNLRKKMERALVELICSFFVASWTGVVKYHGPRPSMRPKQVFVANHTSMIDFIILEQMTAFAVIMQKHPGWVGLLQSTILESVGCIWFNRSEAKDREIVTRKLREHSQGADNNPLLIFPEGTCVNNQYSVMFKKGAFELGCTVCPIAIKYNKIFVDAFWNSRKQSFTMHLLQLMTSWAVVCDVWYLEPQNLRPGETPIEFAERIRDIISVRAGLKKVPWDGYLKYSRPSPKHRERKQQSFAESVLRGLELEEK

SEQ ID NO: 117 - protein, 375 aa, Eucalyptus grandis
MASPRKLPTSSSELDLDRLNIEDYLPSGSSIHEPPGQLRLRDLLDITPTLTEAAGAIVDDSFTRCFKSNSQEPWNWNVYLFPLWCFGVVVRYLILFPARVLVLTIGWIIFLSSFAIVHFMLKAHDALRRKLERLLVELICSFFVASWTGVVKYHGPRPSIRPKQVFVANHTSMIDFIILEQMTAFAVIMQKHPGWVGLLQSTILESVGCIWFNRSEAKDREIVARKLRDHVLGTDNNPLLIFPEGTCVNNHYTVMFKKGAFELGCTVCPIAIKYNKIFVDAFWNSRKQSFTMHLLQLMTSWAVVCDVWYLEPQTLKPGETPIEFAERVRDIISVRAGLKKVPWDGYLKYSRPSPKHREGKQRSFAEWVLQRLEER

SEQ ID NO: 118 - protein, 375 aa, Cucumis sativus
MSGAALLKSSASELDLDRPNIEDYLPSGSSIQQPTAKLRLRDLLDISPTLTEAAGAIVDDSFTRCFKSNPPEPWNWNIYLFPLWCCGVVIRYLFLFPARVLILTIGWIIFLSTFIPVNLLLKGHPKLRAKLERFLVELICSFFVASWTGVVKYHGPRPSIRPKQVFVANHTSMIDFIVLEQMTAFAVIMQKHPGWVGLLQSTILESIGCIWFNRTELKDREIVAKKLNDHVQGADNNPLLIFPEGTCVNNHYSVMFKKGAFELGCSVCPIAIKYNKIFVDAFWNSRKQSFTMHLLQLMTSWAVVCDVWYLEPQVLKPGETPIEFAERVRDIICARAGLKKVPWDGYLKHSRPSPKYRERKQQSFAESVLQLLDNK

SEQ ID NO: 119 - protein, 375 aa, Gossypium arboreum; identical to SEQ ID NO: 118

SEQ ID NO: 120 - DNA, 1116 bp, Cocos nucifera
  source 1..1116: mol_type = genomic DNA
atggttgagctgcggtcatcgagctcggagatggatctggaccgccccaacatcgaggagtacctcaccacggactccatccaagaatcccccaagaagctccacctgagggacttgctcgacatttctcccacgctgacggaggccaccggcgccatcgttgatgattccttcactcgctgctttaaatcgaatcctccagagccctggaattggaatgtctatttatttcccttatggtgcttgggagtgattattagatatggaattctttttcccctaagagttgcaatcttgacagcaggttggctagtgttctttgcagccttcattcctgtacatttcttgttgacagcacataataagtggaggcgtaaaatagagaggaagttggttgagatgatatgcagtgtctttgttgcttcatggacaggggtagtcaagtatcatgggcctcgtcctagcatgcgccctcagcaggtatttgttgccaaccacacttccatgattgatttcatcatactagagcagatgacagcatttgctgttataatgcaaaagcatcctggatgggttggatttattcaaaagaccatattggaaggtgttggttgtatttggttcaaccgtacagaatcaaaggatcgtgaagttgtggcacgaaagttaagagaacatattcatggagctgacaacaaccctcttctgatatttccagaaggaacttgtgttaacaaccattacactgtcatgttcaagaagggtgcttttgaacttggttgtgctgtttgcccggttgcaataaagtacaacaaaatttttgtggatgccttctggaacagtaagaagcaatcttttacaatgcatttgtttcaccttatgacatcgtgggctgttgtttgcgatgtttggtacctggagcctcagtacataagacctggagagacgcccattgaatttgctgaaagggttagagacatgatatctgttcgagctggtctcaaaaaggtcccgtgggatggatatttgaagtacttccgccccagtcctaagctaacagagaggaagcagcagatctttgcggagtcggtcttgcagaggttggaggaaaaataa

SEQ ID NO: 121 - DNA, 1131 bp, Arabidopsis thaliana
  source 1..1131: mol_type = genomic DNA
atgagcagtacggcagggaggctcgtgacttcaaaatccgagcttgacctcgatcaccctaacatcgaagattaccttccttctggttcttccatcaatgaacctcgcggcaagctcagcctgcgtgatttgctagacatctctccaacgctcactgaagctgctggtgccattgttgatgactcgttcacaagatgtttcaaatcaaatcctccagaaccttggaactggaatatttacttattcccactatactgctttggggttgttgttagatactgtatcctctttcccttgaggtgcttcactttagcttttgggtggattattttcctttcattgtttatccctgtaaatgcgttgctgaaaggtcaagataggttgaggaaaaagatagagagggtcttggtggaaatgatttgcagcttttttgtcgcctcatggaccggagttgtcaaatatcacgggccacgtcctagcatccgtcctaagcaggtctatgttgccaaccatacttcaatgattgatttcatcgtattggagcagatgaccgcatttgctgttataatgcagaagcatcctggttgggttggtcttctgcaaagcacaatattagagagtgtgggatgtatctggttcaatcgttcagaggcaaaggatcgtgaaattgtagcaaaaaagttaagggaccatgtccaaggagctgacagtaatcctcttctcatatttcccgaagggacatgtgtaaataataattacacagtgatgtttaagaagggtgcttttgaattggactgcactgtttgtccaattgcaattaaatacaacaagatttttgttgacgccttctggaatagcagaaaacaatcatttactatgcacttgctgcaactcatgacatcatgggctgttgtatgtgaagtgtggtacttggaaccacaaaccataaggcccggtgaaacaggaattgaatttgcagagagggtcagagacatgatatctcttcgggcgggtctcaaaaaggtcccttgggatggatacttgaagtattcgagaccaagccccaagcatagtgaacgcaagcaacagagtttcgcagagtcgatcctggctagattggaagagaagtga

SEQ ID NO: 122 - DNA, 1116 bp, Elaeis guineensis
  source 1..1116: mol_type = genomic DNA
atggttgagctgcggtcatcgagctcggagatggatctggaccgccccaacatcgaggagtacctcccaccgactccatccaagaatccccccaagaagctccacctgagggacttgctcgacatttctcccacgctgacggaggccgccggcgccatcgttgatgattccttcactcgctgctttaaatcgaatcctccagagccctggaattggaatgtctatttatttcccttatggtgcttgggagtgattattagatacggaattctttttcccctaagagttgcaatcttgacagcagggtggctagtattctttgcagcctttattcctgtacatttcttgttgacagcacataataagtggaggcgtaaaatagagaggaagttggttgagatgatatgcagtgtctttgttgcttcatggacaggggtggtcaagtatcatgggcctcgtcctagcatgcgccctcagcaggtatttgttgccaaccacacttccatgattgatttcatcatactagagcagatgacagcatttgctgttataatgcaaaagcatcctggatgggtcggatttattcaaaagactattttggaaggtgttggttgtatttggttcaaccgtacagaatcaaaggatcgtgaagttgtggcacgaaagttaagagaacatattcacggagctgacaacaaccctcttctgatatttccagaaggaacctgcgtcaacaaccattacactgtcatgttcaagaagggtgcttttgaacttggttgtgctgtttgcccagtcgcaataaagtacaacaaaatttttgtggatgccttctggaacagtaagaagcaatcttttacaatgcatttgtttcaccttatgacatcgtgggctgttgtttgcgatgtttggtacctggagcctcagtacataagacctggagagacgcctattgaatttgctgaaagggttagggacatgatttccattcgagctggtctcaaaaaggtgccgtgggatggatatttgaaatacttccgccccagtcctaagctcactgaaagaaagcagcagatatttgcagagtcagtcttgcagcggttggaggaaaaataa

SEQ ID NO: 123 - DNA, 1057 bp, Phoenix dactylifera
  source 1..1057: mol_type = genomic DNA
atggttggactgcggtcatcgagctcggagatggatcttgaccggccgaacattgaggagtacctcaccaccgactccatcgaagaatcccccaagaagctccacttgagggacttgctcgacatttctcccacgctgacggaggccgctggtgctatcgttgatgattctttcactcggtgctttaaatcgaatcctccagaaccctggaattggaatgtctatttatttcccttatggtgcttgggagtgattattagatacggaattctttttcccctaagagttgcagtcttgacagcagggtggctagtattctttgcagcctttattcctgcacatttcctgttgacagctcataataagtggaggcgtaaaatagagaggaagttggttgagatgatatgcagtgtctttgttgcttcatggacaggggtggtcaagtatcatgggcctcgtcctagcatgcgcccccagcaggtatttgttgccaaccacacttcaatgattgatttcatcatactagagcagatgacagcatttgctgttatcatgcaaaagcatcctggatgggtcggatttattcagaagactattttggaaggtgttggttgtatttggttcaaccgtacagaatcaaaggatcgtgaagttgtggcacgaaagttaagagaacatattcaaggagctgacaacaaccctcttctgatatttccagaaggaacctgcgttaacaaccattacactgtcatgttcaagaagggtgcttttgaacttggttgtgctgtttgcccagttgcaataaagtacaacaaaatttttgtggatgctttctggaacagtaagaagcaatcttttacgatgcatttgtttcaccttatgacatcatgggctgttgtttgtgatgtttggtatctggagcctcagtacataagacctggagagacgcccattgaatttgctgaaagggttagagacatgatttctgttcgagctggtctcagaaaggtcccatgggatggatatttgaaatacttccgcccgagtcctaagctaa

SEQ ID NO: 124 - DNA, 1116 bp, Musa acuminata
  source 1..1116: mol_type = genomic DNA
atggctgggttggctacctcgagcacggagatggatctcgaccgccccaacatcgacgagtacctcaccgtggagtcgatccgggaggcccccaagaagctccacctgagggacctcctcgacatttctcctactctcaaagaagctgccggcgccatcgtggacgactccttcactcgttgctttaagtcgaatccttcagaaccctggaattggaatatctatttgttccctttatggtgcttgggagtagttattagatatgggattctttttccattcagagttataatcttggttgcaggatggatagtattctttgcagccttttcactggtgcatttccttttaggagaacataataagtggaagcgtgaaatagagaggaaactggttgagatgatatgcagcgtatttgttgcttcatggacggcagtgattaaataccatggacctcgtcccagcatgcgccctcaacaggtcttcgttgccaaccacacttctatgattgatttcatcatcttagaacagatgacagcatttgctgtcattatgcaaaagcatcctggttgggttggatttatccagaagatcatcgtagaaagtttaggttgtatatggttcaaccgtacagaggctaaggaccgtgaaattgttgctagaaagttgagagaacacattcaaggagttgacaacaaccctcttctgatatttcctgagggaacttgcgttaacaaccattatactgttatgttcaagaagggtgcttttgaacttggttgtgctgtttgtcctgtagcaatcaagtacaacaagatttttgtggatgctttctggaacagcaaaaagcaatctttcacgatgcacttagtacagcttatgacatcatgggctgttgtttgtgatgtttggtacctggagcctcagtatataaggcctggagagactcctattgaatttgctgaaagggttcaagacatgatctctgttcgagctggtctcaaaaaggtcccatgggatggctatctaaagtacttccgccccagtcccaagctcatagagcgcaagcagcagatctttgcggagtcagtcttacagcgattggaggagaaatga

SEQ ID NO: 125 - DNA, 1119 bp, Ananas comosus
  source 1..1119: mol_type = genomic DNA
atggctgaagctctgggctcgtcgagcgcggagatggatctcgaccgtcccaacctcgaggagtacctccccaccgactccatccaagactcccccaaaaacctccacctgagggacctgctcgatatctcccccacgctcaccgaggccgcgggcgccatcgttgatgactcattcactcgctgctttaaatcaaatcctccagaaccatggaattggaatatatatttgttccctctatggtgcctcggagtcgttgtaagatatgggattctttttccactcagagttgcagtcttggcgatagggtggatagtatttttttctgccttcttccctgtacatttcttattgaaagggtatcccaagtggaggcgcaaactagagagaaaattggttgagatgatgtgcagtgtatttgttgcttcatggactggagtcgtaaaatatcatggaccacgcccaagcacgcgccctcatcaggtatttgttgctaatcacacctccatgatcgatttcatcattttagaacaaatgactgcatttgctgttatcatgcaaaaacatcctggatgggttggatttattcagaagaccatcttagaaagtgtaggatgtatttggttcaaccgaacggagtctaaggatcgcggagttgtcgggcggaagctaagagaacatgttcaaggagtagacaacaaccctcttctgatatttccagaaggaacctgcgtaaacaatcactacactgtcatgtttaagaagggtgcttttgagcttggatgtgctgtttgcccaatagcaatcaaatacaacaaaatttttgtggatgccttctggaacagtaagaagcaatcttttaccatgcatctggtccgcctcatgacgtcgtgggctgttgtctgtgatgtgtggtacttggagcctcagtacctgagacctggggagacgccaattgaatttgctgaaagggttagagacatgatttctgctcgagctggtctaaagaaggttccatgggatgggtatctgaagtactttcgtcctagccccaagcatacagaacggaagcagcagatctttgcagagtcaatcttgcggcggttggagaggaaatga

SEQ ID NO: 126 - DNA, 1113 bp, Asparagus officinalis
  source 1..1113: mol_type = genomic DNA
atggcggggctggagtcctcgagcgcagggatcgacgtcgaccctccaaatattgaagactatctcacatccgatgccctccatcaacctcataagaagcttcaattgaaggatttactcgatatttctcctacactaactgaggctgcaggagcaattgttgatgactcatttacacgatgtttcaagtcaaatcctcccgaaccctggaattggaatgtctacctatttcccttgtggtgcttgggagtggttgttcgatatgggatcctttttcccttgagagttatgactctggcagctggatggattgtgttcttttcagcctttcttcctgttcattatctaatgaaagggcagaacaaatggaaaaataatatagagagaaaattggtggagatgatatgtagtgtttttgttgcttcttggactggtgttgtcaggtatcacggacctcgtcctagcatgcgccctcaacaggtatttgtggcgaatcatacttcgatgattgatttcatcattttagagcagatggctgcatttgctgtaatcatgcagaagcatcctggatgggttggtttccttcagacgacaattttggaaagcataggttctatttggttcaatcgtaccgaggccaaggatcgcgaagttgtagcaagaaagttaagagaacatactgaaggggacaacaatcctttactaatatttccggaaggaacttgtgtgaacaatgactacactgttatgttcaaaaagggcgcatttgaactgggatgtgctgtttgtcctgtagccatcaagtacaataaaattttcgtggacgccttctggaacagcaagaagcaatcttttacgatgcatctgatgcgccttatgacatcatgggctgtggtatgtgatgtttggtatcttgaaccacagtatctgaaaccgggagagacttctattgaattcgctgaaagggtcagggatatgatttcggtccgagctggtctcagaaaggtcccgtgggatggatatttgaagtacttccgcccaagtcctaagcttacagagcgcaagcagcaaatatttgcggaatcagtcctacggcggctggaagaaaagtga

SEQ ID NO: 127 - DNA, 1113 bp, Oryza brachyantha
  source 1..1113: mol_type = genomic DNA
atggcgtcctcatcggtggcgggggacatcgagctggaccggccgaacctggaggattacctcccgcccgactcgctgccgcaggaatcccccgggaatctccatctgcgcgatctgcttgacatctcgccggtgctcactgaagcggcgggggcggccgtcgatgattcattcacacgttgctttaagtccaattctccagagccatggaattggaacatttatttattcccactatggtgcttgggagtagtgataagatatggaatactcttcccactaaggggcttaactcttctagttggatggatagctttcttcgctgcctttttctctgtgcatttcttatttaaagggcaaaagatgagaagtaaaatagagagaaaactggttgaaatgatgtgcagtgtttttgttgcttcttggactggagtgatcaagtatcatggacctcgcccaagcacacggcctcatcaggtatttgttgcaaaccacacatcgatgatagacttcattattctggagcagatgacagcatttgctgtcattatgcaaaagcatcctggatgggttggatttattcagaagactatattggaaagtgtgggttgcatctggttcaatcgtaacgatctcaaggatcgtgaagtagtagcaaaaaagttacgagatcatgttcaacatccagacaacaatcctctcctaattttccctgaaggaacttgtgttaacaaccagtacactgtcatgttcaagaagggtgcttttgagcttggctgtgctgtatgcccaatagctatcaaatacaataaaatatttgttgatgccttctggaatagtaagaagcaatcttttacaatgcacttggttcggcttatgacatcatgggcagttgtgtgtgatgtatggtacttggagccgcaatatctaaaggagggagaaacagcaattcaatttgctgaaagagtaagagacatgatagctgctagagctggtcttaagaaggttccatgggacggatatctgaaacacaaccgccctagccccaaacacactgaagagaagcagcgcatctttgctgattctgtgttgcagagactggaggaaagctaa

SEQ ID NO: 128 - DNA, 1113 bp, Oryza sativa
  source 1..1113: mol_type = genomic DNA
atggcgacctcgtcggtggcgggggacatcgagctggaccggccgaacctggaggactacctcccatccgactcgctgccgcaggagttccccaggaatctccatctgcgcgatctgctggacatctcgccggtgctcactgaagcggcgggcgccatcgtcgatgattcattcacacgttgctttaagtcaaattctccagagccatggaattggaacatttatttattcccattgtggtgcttgggagtagtgataagatacggaatactattcccgctgaggggcctaactcttctagttggatggttagcattctttgctgccttttttcctgtacatttcttattgaaaggtcaaaagatgagaagtaaaatagagagaaagctggttgaaatgatgtgcagtgtttttgttgcttcttggactggagtgatcaagtatcatgggcctcgcccaagcacacggcctcatcaggtatttgttgcaaaccatacatcgatgatagatttcattattctggagcagatgacagcatttgctgtcattatgcaaaagcatcctggatgggttggatttattcagaagactatcttggaaagtgttggttgcatctggtttaatcgcaatgatctcaaggatcgtgaagtggttgcaaaaaagttacgagatcatgttcaacatccagacagcaatcctctcctgattttccctgaaggaacttgtgttaacaaccagtacactgtcatgttcaagaagggtgcttttgagcttggctgtgctgtatgcccaatagctatcaaatacaataaaatatttgttgatgccttctggaatagtaagaagcaatcgtttacaatgcacttggttaggcttatgacatcatgggcagttgtgtgtgatgtatggtacttggagcctcagtatctgagggatggagaaacagcaattgaatttgctgaaagagtaagagacatgatagctgctagagctggtcttaagaaggttccgtgggacgggtatctgaaacacaaccgccctagtcccaaacacactgaagagaagcagcgcatctttgctgactctgtgttgcggagactggaggaaagctaa

SEQ ID NO: 129 - DNA, 1092 bp, Nelumbo nucifera
  source 1..1092: mol_type = genomic DNA
atggacttggatcgaccaaacatagaggaatatttaccttcagaagccattcaagagtctaacgagaagcttcacttgcgtgatttgctcgacatttcgcctactctaaccgaggctgctggtgccattgttgatgattctttcactcgttgtttcaagtcaaatccgtcagaaccttggaattggaatgtatatttatttccactttggtgctttggagtggtggtaagatatgggattctttttcctgttagagttctagtgttaacaattgggtggataatattcctttcatccttcattcctgcacatttcctattgagaagtcatgataagtggaggaagaagatagagagatatctggtggagttaatatgcagcttctttgttgcatcatggactggggttgtcaaatatcatgggccacggccaagcatgcgacccaagcaggtttttgtggccaatcatacttccatgatagattttattgttttagaacagatgactgcatttgctgtaattatgcagaagcatcctggatgggttgggcttttgcaaagcactattttggagagtgtaggttgtatctggttcaatcgtgcagaagcaaaggaccgtgaaattgtagcaagaaagttaagagaccacattcaaggggttgacaacaatcctcttcttatatttccagaaggaacatgtgtaaataaccactatacagtcatgttcaagaagggtgcatttgaacttggatgcactgtttgtccaatagcaatcaagtacaataaaatttttgttgatgccttctggaatagtaagaagcaatcttttaccatgcacttactgcaccttatgacttcatgggctgttgtttgtgatgtttggtatttggagccgcaaaatattagacctggagagacacccatagaatttgcagagagggtacgagacataatttctgttcgagcaggtcttaaaaaggttccatgggatggatatttgaaatattctcgtcctagccccaaacacagagaaagaaagcaacaaaggtttgtagagtcggtattgcagcgcttggagaaaaagggaaaatga

SEQ ID NO: 130 - DNA, 1131 bp, Vitis vinifera
  source 1..1131: mol_type = genomic DNA
atggccaacgctcccgataataagctcacttcctcaagctccgagctcgacttggatcgccccaatctcgaagactaccttccctccggatccatgcaagaacctcgcggcaagcttcgcctgcgtgatttattggacatttcgccgaccctaaccgaggctgctggggccattgttgacgactctttcacacgatgtttcaagtcgaaccctccggagccttggaactggaatgtgtatttgtttcctctttggtgtttgggagtggtaattcgatatggaattttatttcccacaagggttctagtactcacactggggtggataatattcctttcatcctttattccagtacattttctattgaagggaaacgataagttgaggaaaaagttggagagatgtctagtggagttaatttgcagcttctttgttgcatcatggactggagttgtcaagtaccatgggccacggcctagcaggaggcctcagcaggtttttgttgccaatcatacttccatgattgattttatcgttttagaacagatgactgcatttgcagttattatgcagaagcatcctggctgggttggattgctgcaaagtaccattttggagagtgtaggatgtatctggttcaatcgtacagaagcaaaggaccgtgaaattgttgctaggaagctaagggatcatgttcagggggctgacaacaaccctcttctcatattcccagaaggaacttgtgtgaataaccactacactgtcatgttcaagaagggcgcattcgaacttggctgcactgtttgccctattgcaataaagtacaataagattttcgttgatgctttctggaacagtaagaagcaatcctttacaatgcatcttctgcagcttatgacatcctgggctgtcgtttgtgatgtttggtacttagagccccaaacattgaagccaggagagacacccattgaatttgcagagagggtcagggacataatttctcttcgagctggtttgaaaaaggttccttgggatggatatttgaagtactctcgccctagcccaaagcatagagagcagaagcagcagagctttgctgattcagtgttacggcgcctggaagagaagtga

SEQ ID NO: 131 - DNA, 1122 bp, Nicotiana tomentosiformis
  source 1..1122: mol_type = genomic DNA
atgaatatgaataagctaaaaacatcaagctccgaattggacttggatcgacccaatctcgaagattatcttccaactggatccatcccagaaccccatggcaagcttcgcctgcgtgatttaattgatatttctcccaccctaactgaggctgctggtgccattgttgacgattctttcaccagatgcttcaagtcaaatccaccagagccttggaactggaacatttatttgttccctttatggtgcttgggggttgttgttagatatgggattcttttccctataagagttattgtcttgacaataggatggataatattcctctcttgctatatcccggtgcatttcctgctgaaaggacacgataagttcaggaaaaagcttgagagatgtctggtggagctgatatgcagtttctttgttgcatcttggactggggttgtcaaataccatggtccacggcctagcatacgacctaagcaggtttttgtggcgaatcacacgtcaatgatagattttattgtcctagagcagatgactgcatttgcagtgatcatgcagaagcatcctggatgggttggactactgcagagtaccattttagaaggtgttggatgtatctggttcaaccgctcagaagccaaggatcgtgaaattgtagcacgaaagttgaggcaacatgttgaaggggccgataacaaccctcttcttatattccccgagggaacttgcgtaaataaccactacactgtcatgttcaaaaagggagcatttgaactcggttgcactgtttgtcctgttgcaatcaagtacaacaaaatttttgttgacgccttttggaatagtagaaaacaatccttcacaatgcacctcttgcagctcatgacatcttgggctgttgtctgtgatgtttggtacctggagcctcagaacataagacctggggagactccaatcgagtttgcagagagggtgagggacatcatttctgctagagcaggtcttaaaaaggttccttgggatggatatttaaaatactctcgtcctagccccaagcatcgagagaggaagcaacagagttttgcagaatcagtgctgcgtcgcctggaagagaagtag

SEQ ID NO: 132 - DNA, 1128 bp, Jatropha curcas
  source 1..1128: mol_type = genomic DNA
atggctactccaggtaagctaaagacctcgagctctgaattggacttggatcgacccaatatcgaagactaccttccttctggagtctctattcaagaacctcgtggcaagcttcgtctgcgtgatttgcttgacatttcgccgaccctaacggaggctgctggtgccattgttgatgacacctttacaaggtgtttcaagtcaaatcctccagaaccatggaattggaacatatatctatttcccctttggtgctgcggtgtggtgtgtcgatatgggattttgtttcccatcagggttctagtactgacaatagggtggataattttcctttcatgctacattcctgtgcatttcctacttaaaggacatgacaagttgagaaaaaagcttgagagatgtttggtggagttaatttgcagcttctttgtggcatcatggaccggagttgtcaagtaccatggtccacggcctagcatccgacctaaacaggtttttgtggccaatcatacctccatgattgattttatcatcttggaacagatgactgcatttgctgttattatgcagaagcatcctggatgggttggactactgcaaagcactatattagagagtgtcggatgtatctggttcaatcgttcagaggcaaaggatcgtgaaattgtaacaaaaaagttaagggatcatgtacagggggctgacaataaccctcttctcatatttcctgaaggaacttgtgtaaataatcactatactgtaatgttcaagaagggtgcattcgaactgggatgtactgtttgtccaattgcaatcaaatacaacaaaatttttgttgatgctttttggaacagccggaagcagtcatttacaacgcatttgctgcaactcatgacttcctgggctgttgtttgtgatgtatggtacttggagccacaaaatctgaaacctggagagacacccattgagtttgctgagagggtcagggacataatatctgtacgagcaggtctcaaaaaggttccttgggatggatatctaaagtattctcgccctagcccaaagcatagagagcgaaagcaacaaagctttgctgagtcagtgctgcagcgactggaggagaaatga

SEQ ID NO: 133 - DNA, 1131 bp, Glycine max
  source 1..1131: mol_type = genomic DNA
atgaataactcagggacacccaagtcttcaagttctgaattggatcttgatcgacccaacattgaggattacctcccttcagggtccaccattcaacaagaacctcatggaaagcttttcctgcatgatttgctcaatatttctcctactttgtctgaggctgcaggtgctattgtagatgactcattcacaagatgcttcaagtcaaatcctccagaaccatggaattggaatgtttatttgtttcctttgtggtgttttggagttgtgattcgatacttgattctgttcccaatcagggttatagggttaacaataggatggataatatttctttcatccttcattccggtgcacttcctattgaaaggacatgacaagttaaggagaagtattgagaggtctttggtagagatgatgtgcagtttctttgttgcatcttggactggggttgttaagtatcatggacccaggcctagcaggagaccaaagcaggtttttgtagccaaccatacttccatgattgatttcattatcttagaacagatgactgcttttgctgttattatgcagaagcatcctggatgggttggattattgcagagtaccattttggagagtctaggatgcatctggttcaaccgtacagaggcaaaggatcgggaaatagtagcaaggaaattgagggatcatgtccagggagctgataacaacccccttctcatatttcctgaaggaacttgtgtaaataatcactatacagtcatgttcaagaagggtgcatttgaacttggctgcacagtttgcccagttgcaatcaagtacaataagatttttgtagatgctttttggaatagtcgaaagcaatcattcactatgcatctgttgcagctaatgacgtcttgggcagttgtttgtgatgtttggtacttggagccacaaaatctgaagccaggagagacgcctattgagttcgcagagagggtgagagacataatctcagttcgtgctggccttaaaaaggttccttgggatggatatctgaagtattctcgtcctagcccaaagcatagagaaaggaagcaacagaactttgctgagtcagtgctgcggcgatgggaggaaaagtga

SEQ ID NO: 134 - DNA, 1116 bp, Sesamum indicum
  source 1..1116: mol_type = genomic DNA
atgagtaagcttaacacatccagctccgaattggattttgatcgccccaacatcgaggactatctcccatccggatccattcaagagcctcacggcaaactccgcctgcgtgatttgctcgatatttcaccaactctcactgaggccgctggtgcaattgttgatgactctttcaccagatgcttcaagtcaaatcctccagaaccctggaactggaacatatacttgtttcctttatggtgcttgggagtggtcatcagatatggccttcttttcccattaagggtaatagtgttgacaataggatggattatatttctatcatgctattttcctgtgcatttcctgttaagagggcacgacaaattgaggaaaagattagagagaggtctagtggagttgatttgcagtttcttcgttgcatcatggacaggggttgtcaagtatcatggtccacggccgtccatgcgacctaagcaggtttttgtggcgaatcacacatccatgattgatttcattgttttggaacaaatgactgcttttgcagtgattatgcagaagcatcctgggtgggttggattattgcagagcacaattttggaaagtctaggatgtatctggttcaaccgctcagagtccaaggatcgtgaaattgttgcaaaaaagctaagggaacatgtccatgatgctgataacaatcctcttcttatattcccggaaggaacttgtgtgaataaccattacactgttatgtttaagaagggtgcatttgaacttggctgcactgtctgtccaatagcaatcaagtacaacaagatatttgttgatgccttctggaatagccgaaagcaatccttcactacacacttgttgcagcttatgacatcctgggctgttgtttgtgacgtttggtacctagagcctcaaaatctgaaacctggggaaacacccattgaatttgcagagagggtgagggacattatttctgttcgggccggcctcagaaaggtgccttgggatggatatttgaagtactctcggcctagtccgaagcatcgtgaacgcaagcaacaaagctttgcagagtcaattctccgtcgcttggaagagaaatag

SEQ ID NO: 135 - DNA, 1095 bp, Brachypodium distachyon
  source 1..1095: mol_type = genomic DNA
atggcgtcgtcgctcgacgcgccgaaccttgatgattacctccccacggactcgctcccgcaggaaccccccaggagcctcaatctgcgcgatctgctggacatctcgccagtgctcactgaagcggcgggcgccatcgtggatgattcgttcacacgctgctttaagtcaaattctccagagccatggaactggaacatttatttgttcccgttatggtgcttcggagtagtcgtaagatacggactactgtttccactcagggtattaacgcttggattaggatggatggtattctttgctgccttttttcccgtgcatttcctattgaaagggcaaaataaactgagaagtaaaatagagagaaagctcgttgaaatgatgtgcagtgtttttgttgcttcttggactggagtaatcaagtaccatggaccacgcccaagctcacggccttatcaggtatttgttgcaaaccatacatcaatgatagatttcattattctggagcagatgacagcatttgctgtcattatgcaaaagcatcctggatgggttggatttattcagaagactattttggaaagtgtgggttgcatctggtttaatcgtaatgatcttaaggatcgtgaagtagttggcagaaagttacgtgatcatgttcaacgtccagacaacaaccctctcttgattttcccagaaggaacttgtgttaacaaccagtacactgtaatgttcaagaagggtgcttttgagcttgggtgtgctgtatgtccgatagctatcaagtataataaaatatttgttgatgccttctggaatagtaagaagcaatctttcacaatgcacttgggtcggcttatgacatcatgggctgtagtgtgtgatgtttggttcttggaacctcaatatctcagggaaggagagacatcgattgcatttactgaaagagtaagggacatgatagctgctcgagccggtcttaagaaggttctgtgggatgggtatctgaagcataaccgtcctagccccaaacacactgaggagaagcagcgcatatttgcagaatcggtgttgaagagactagaggaaagctaa

SEQ ID NO: 136 - DNA, 1116 bp, Setaria italica
  source 1..1116: mol_type = genomic DNA
atggcgagctcctcggtggcggcggacatggagctggaccgccccaatctggaggactacctcccgcccgactcgctcccgcaggaggcgccccggaatctccatctgcgcgatttgctggacatctcgccagtgctcaccgaggcagcaggcgccatcgtcgatgactccttcacgcgttgctttaagtcaaattctccagagccatggaattggaacatatatctgttccccttatggtgcttgggtgtagtaataagatatggaatactcttcccactgaggtccttaacgcttgcaataggatggttagcattttttgctgccttttttcctgtccatttcctattgaaagggcaagacaagttgagaagtaaaattgagaggaagttggttgaaatgatgtgcagtgtttttgttgcttcatggactggagtgatcaagtatcatggaccacgcccaagcacacgacctcatcaggtattcgttgcaaaccatacatcaatgatagatttcattattctggagcaaatgacagcatttgctgtcatcatgcagaagcatcctggatgggttggatttattcagaagactatcttggaaagtgtcggttgcatctggtttaatcgtaatgatcttcgggatcgtgaagttacggcacggaagttacgtgatcatgttcaacaaccagacaaaaatcctctcttgatttttccggaaggaacttgtgttaacaaccagtacacggtcatgttcaagaagggtgcctttgagcttggctgcgctgtctgtccaatagctatcaagtacaataaaatatttgttgatgccttttggaacagtaagaagcaatcttttacaatgcacttggtccggctgatgacatcatgggctgttgtgtgtgatgtttggtacttacctcctcaatatctgagggagggagagacggcaattgcatttgctgagagagtaagggacatgatagccgctagagctggactaaaaaaggttccgtgggatggctatctgaaacacaaccgtcctagtcccaaacacactgaagagaaacaacgcatatttgccgaatctgtcctgatgagactggaggagaaatga

SEQ ID NO: 137 - DNA, 1131 bp, Cicer arietinum
  source 1..1131: mol_type = genomic DNA
atgaatagcactggaacacttaagtcttcaagttctgagttggatcttgatcgacccaacattgaggattatctcccttcaggagccgccattcaacaagaacctcgcggcaagcttcgcctgcatgacttgcttgatatttctcctacactatctgaggcagctggtgctattgtagatgactcattcacaagatgtttcaagtcaaatcctccagaaccatggaattggaatatatatttgtttcctttgtggtgttttggagttgttgttcgatatttgatactgttccctacaagggttcttgggttaacattaggatggataatatttctttctgctttcattccagtgcacctcctattgaaaggacatgacaagttgaggagaaatattgagaggtctttggtagagatgatgtgcggtttctttgttgcatcttggactggggttgtcaagtaccatgggccaaagcccagcaggcgaccaaaacaggtttttgttgccaaccacacttccatgattgatttcattatcttagaacagatgactgcttttgctgttattatgcagaagcatcctggatgggttggattgttgcaaagcaccattttggagagtgtaggatgtatctggttcaatcgcacagaggcaaaggatcgagaaattgtggcaagaaaattgagggaacatgtccagggagctgacaacaatcctcttctcatatttccagagggaacttgtgtaaataatcactacacagtcatgtttaagaagggtgcatttgaacttggctgcacagtttgccctgttgcaatcaaatacaacaaaatttttgtcgatgcattttggaatagtcgaaagcaatcattcactaaacatctgttgcagctaatgacatcatgggctgttgtttgtgatgtttggtacttggagccacaaaacctaaagccaggagagacaccaattgagtttgccgaaagggtgagagacataatctcacatcgtgctggtcttaaaaaggttccttgggatggatatctgaagtattcgcgtcctagcccaaagcatagagaaagaaagcaacagaactttgctgagtcggtgctgcggcgtttggaagaaaaataa

SEQ ID NO: 138 - DNA, 1116 bp, Zea mays
  source 1..1116: mol_type = genomic DNA
atggcgagctcgtctgtggcggcggacatggagctggaccgccccaacctggaggactacctcccgcccgactcgctcccgcaggaggcgcccaggaatctccatctgcgcgatctgcttgacatctcgccggtgctaaccgaggcagcgggtgccatagtcgatgattcattcacgcgctgctttaagtcgaattctccagaaccatggaactggaacatatatttgttccctttatggtgcttcggtgtagtaattcgatatggattactcttcccactgaggtccttaacgcttgcaataggatggttagcattttttgctgcctttttccccgtgcatttcctattgaaaggtcaagacaagttgagaaataaaattgagaggaagttggttgaaatgatgtgcagtgtttttgttgcttcatggactggagtgatcaagtaccatggaccacgcccaagcacacgacctcatcaggtatttgttgcaaaccatacatcaatgatagatttcattattctggagcaaatgacagcatttgctgtcatcatgcagaagcatcctggatgggttggatttattcagaagactatcttggaaagtgtgggttgcatctggtttaaccgtaatgatctccgggatcgtgaagttacggcacggaagttgcgtgatcatgttcaacatccagacaaaaaccctctcttgattttcccagaaggaacttgtgttaacaaccagtatacggtcatgttcaagaagggtgcctttgagcttgggtgtgctgtctgtccaatagctatcaaatacaataaaatatttgttgatgccttttggaacagtaagaagcaatcttttacgatgcacttggtccggttgatgacatcatgggctgttgtgtgtgatgtttggtacttggagcctcaatatctgagggagggagagactgcaattgcgtttgctgagagagtaagggacatgatagcagctagagctggtcttaagaaggtcccgtgggatggctatctgaaacacaaccgccctagtcccaaacacaccgaagagaagcaacgcatattcgccgaatctgtcttgaggagactagaggagaaatga

SEQ ID NO: 139 - DNA, 1137 bp, Gossypium hirsutum
  source 1..1137: mol_type = genomic DNA
atgaacagtagtgaagggaagttgaaatcatcgagttccgaattggatttggatcgacccaacatcgaagattatctcccttctggatcttccattcaagaaccacatggcaagcttcgcctgcgggatttgcttgatatttctcccgctttaactgaagctgctggtgctattgttgatgattctttcacacggtgttttaagtcgaatcccccggaaccgtggaactggaatgtgtatctttttcctctctggtgttgtggtgtggtatttcggtacttgattttgttccctatgagggctttaattttgacaataggatggataatatttctgtcatgcttcattcctgtgcactttcttctcaaagggaacgataacttgcggaaaaagatggagagggcgttggtggagctaatctgcagcttctttgttgcatcctggactggagttgttaagtaccatggaccgcggcctagcatgcggcccaagcaggtgtttgtggccaatcatacttctatgattgatttcatcatattagaacagatgactgcatttgctgtcattatgcagaagcaccctggatgggttggactgctacagagcactattttagagagtgtagggtgtatttggtttaaccgttcagaggccaaagatcgtgaaattgtaacaaggaagttaagggagcatagtcagggagctgacaataaccctcttctcatatttcccgaagggacatgtgtaaacaatcaatacagcgttatgttcaagaagggtgcattcgaacttggttgcactgtttgcccgattgcaataaagtacaataaaatttttgttgatgccttttggaatagccggaagcagtcctttacaatgcatttattgcagcttatgacatcctgggctgttgtttgcgatgtttggtacctagagccccaaaatctaaggcctggagaaacacccatcgagtttgcagagaggatcagagacataatctctgttcgagcaggtcttaaaaaggttccatgggacggatatttgaagtattctcgcccgagccctaagcatagagagcgaaaacaacaaagttttgccgaatctgttcttcggggactggaactggaagaaaaatga

SEQ ID NO: 140 - DNA, 1128 bp, Eucalyptus grandis
  source 1..1128: mol_type = genomic DNA
atggcgagccccaggaagctgccgacctcgagctccgagctggacctggatcgcctcaacatcgaggattacctcccttccggatcctccatccacgagccccccggccagctccgcctgcgcgatttgcttgatatcacgccgactctgaccgaggccgccggtgctatcgtcgatgactcgttcacgcggtgcttcaagtcgaattcgcaggaaccgtggaactggaacgtgtacctcttcccgctgtggtgcttcggggtggtggttcggtacttgatcctgttcccggcaagggttttagtgttgacaattggatggataatattcctctcatcatttgccattgttcactttatgcttaaagcacatgatgcactgagaaggaagctggagaggttgctggtggagttaatttgcagcttctttgttgcttcatggactggtgtcgtcaaataccatgggccacggcctagcattcggcctaaacaagtttttgttgccaaccacacttccatgattgatttcatcatcttagagcaaatgactgccttcgctgttattatgcaaaagcatcctggatgggttggactactgcaaagcactattttggagagtgtaggatgcatctggtttaatcgttctgaggccaaagatcgtgaaattgtggcaagaaagttgagagatcacgtactgggaactgataacaatcctcttctcatatttcctgaagggacttgtgtgaacaatcactatactgtcatgttcaaaaagggtgcatttgagcttgggtgcactgtttgccctatcgcaatcaagtacaataagatcttcgtggatgccttttggaacagcaggaaacaatctttcacaatgcatctactgcaacttatgacatcttgggctgttgtttgtgacgtctggtacttggaaccccaaaccttgaaacctggtgaaacgccaattgaatttgcagagagggtccgtgacatcatatctgttcgagctggtttgaagaaggttccttgggatggatatctgaagtactctcgccctagccccaagcatagagaagggaagcaacgaagctttgctgagtgggtgctgcagcgacttgaggagaggtga

SEQ ID NO: 141 - DNA, 1128 bp, Cucumis sativus
  source 1..1128: mol_type = genomic DNA
atgagtggtgctgctcttctcaaatcctccgcctctgaattggacttagatcgacccaatatcgaagattacttgccttccggatcctctatccaacaacccactgccaagcttcgccttcgtgatttgctcgatatttcgccgacccttaccgaggctgctggtgctattgttgatgattcgtttacaaggtgtttcaaatcaaacccaccagagccatggaattggaatatttatttgttccctttgtggtgctgtggagtggtgattcggtatttgtttctcttcccggcaagggttctcatattgacgataggatggataattttcctttcaacgttcattccagtgaatctccttctgaaagggcatcctaaactgagagctaagttagagaggtttttggtggagttgatttgcagcttctttgttgcatcttggactggagttgttaagtatcatgggccacggcctagcatcagaccaaaacaggttttcgtggccaaccacacttccatgattgatttcatagtcttggagcaaatgactgcctttgctgttattatgcaaaaacatcctgggtgggttggactgttgcaaagcactatattggagagtataggatgtatatggttcaaccgtacagagttgaaggaccgtgaaattgtagcaaagaagttaaatgaccacgttcaaggggctgacaacaatcctcttcttatatttcctgaaggaacttgtgtaaataaccactactctgttatgttcaagaagggtgcatttgaacttggatgctctgtttgcccaattgcaatcaaatacaataaaattttcgttgatgctttttggaacagcaggaagcagtcgttcactatgcatctgctgcagctcatgacctcttgggctgttgtttgtgatgtttggtacctggagccccaagttttgaagcctggagaaacacccattgagtttgcagaaagggtcagggacataatatgtgctcgagcaggtcttaagaaggttccatgggatggatatttgaagcactcccgtccgagcccaaaataccgagaacgtaaacaacaaagcttcgcggagtcagtgctgcagctattggacaataagtga

SEQ ID NO: 142 - DNA, 1137 bp, Gossypium arboreum
  source 1..1137: mol_type = genomic DNA
atgaacagtagtgaagggaagttgaaatcatcgagttccgaattggatttggatcgacccaacatcgaagattatctcccttctggatcttccattcaagaaccacatggcaagcttcgcctgcgggatttgcttgatatttctcccgctttaactgaagctgctggtgctattgttgatgattcattcacacggtgttttaagtcgaatcccccggaaccgtggaactggaatgtgtatctgtttcctctctggtgttgtggtgtggtatttcggtacttgattttgttccctatgagggctttagttttgacaataggatggataatatttctgtcatgcttcattcctgtgcactttcttctcaaagggaacgataacttgcggaaaaagatggagagggcgttggtggagctaatctgtagcttctttgttgcgtcctggactggagttgttaagtaccatggaccacggcctagcatgcggcccaagcaggtgtttgtggccaatcatacttctatgattgatttcatcatattagaacagatgactgcatttgctgtcattatgcagaagcaccctggatgggttggactgctacagagcactattttagagagtgtagggtgtatttggtttaaccgttcagaggccaaagatcgtgaaattgtaacaaggaagttaagggagcatagtcagggagctgacaataaccctcttctcatatttcccgaagggacatgtgtaaacaatcaatacagcgttatgttcaagaagggtgcattcgaacttggttgcactgtttgcccgattgcaataaagtacaataaaatttttgttgatgccttttggaatagccggaagcagtcctttacaatgcatttattgcagctaatgacatcctgggctgttgtttgcgatgtttggtacctagagccccaaaatctaaggcctggagaaacacccatcgagtttgcagagaggatcagagacataatctctgttcgagcaggtcttaaaaaggttccatgggacggatatttgaagtattctcgcccgagccctaagcatagagagcgaaaacaacaaagttttgccgaatctgttcttcggggactggaactggaagaaaaatga

SEQ ID NO: 143 - protein, 215 aa, Elaeis guineensis
MPDSDNESGGQNNSHNNNVGEYSSSREQDRFLPIANVSRIMKKALPANAKISKDAKETVQECVSEFISFITGEAADKCQREKRKTINGDDLLWAMTTLGFEDYVDPLKVYLHRFREMEGDKCSAGASASSQPQHKDGGDGGGGGGGGAPSMGNNVVGLGGGGGGAGGMMMMMGQQMYATPPSYHHHMSTMSGKSSMGGGSSASSSSPGFGRQGRV

SEQ ID NO: 144 - protein, 133 aa, Elaeis guineensis
MEPENPELNLDLALQPSSPPEPARVFSCNYCQKKFYSSQALGGHQNAHKLERSLAKRSWELATALRPHAGSTIGQHTSTVVLVERQREECCYNGVGLATRGREASRASIRLGSRKESDDKRELADGIDLSLRL

SEQ ID NO: 145 - protein, 190 aa, Arabidopsis thaliana
MGDSDRDSGGGQNGNNQNGQSSLSPREQDRFLPIANVSRIMKKALPANAKISKDAKETMQECVSEFISFVTGEASDKCQKEKRKTINGDDLLWAMTTLGFEDYVEPLKVYLQRFREIEGERTGLGRPQTGGEVGEHQRDAVGDGGGFYGGGGGMQYHQHHQFLHQQNHMYGATGGGSDSGGGAASGRTRT

SEQ ID NO: 146 - protein, 161 aa, Arabidopsis thaliana
MADSDNDSGGHKDGGNASTREQDRFLPIANVSRIMKKALPANAKISKDAKETVQECVSEFISFITGEASDKCQREKRKTINGDDLLWAMTTLGFEDYVEPLKVYLQKYREVEGEKTTTAGRQGDKEGGGGGGGAGSGSGGAPMYGGGMVTTMGHQFSHHFS

SEQ ID NO: 147 - protein, 150 aa, Arabidopsis thaliana
MDYQPNTSLRLSLPSYKNHQLNLELVLEPSSMSSSSSSSTNSSSCLEQPRVFSCNYCQRKFYSSQALGGHQNAHKLERTLAKKSRELFRSSNTVDSDQPYPFSGRFELYGRGYQGFLESGGSRDFSARRVPESGLDQDQEKSHLDLSLRL

SEQ ID NO: 148 - protein, 398 aa, Elaeis guineensis
MASASESRNVTSEETEVTSERRPEEGKEERELGLEFPLMRQSSIYSLTLDEIQNTVCEPGKSFGSMNMDEFLTNIWNVEEGQIASANAQNQQHIGGGGPPAAPPLQRQGSIAVPAPLCRKTVDEVWSDIHRGQNARRQNVDRPPPPSQQQESNCAAPRKPTFGEITLEDFLVKAGVVREGYQPGSAPSAHAPVPPATQYGMPAGYQMVGTEGAPVFGHVVGVQAYGDHQVTAANAMYPVVGDGGGPGYAVGNGFGGRVGNGYGAVAAVGGSPASPGSSEGVGGGQVENSGAAEGGGGGKGGRKRPLDGTVEKVVERRQRRMIKNRESAARSRARKQAYTVELEAELNQLKEENARLKEAEKKMLALKKQLLMEAMAERARVNAQKTILTMRRCNSSKW

SEQ ID NO: 149 - protein, 272 aa, Elaeis guineensis
MEQSTQPSHPVMGIVTGAAQIAYAAPTYQSAAMVTGAPAVIGAIPSPAQPTSTFPTSPAQLTSQHQLAYQQVRQFHHQQQQQQQQQLQTFWANQMLEIEHATDFKNHSLPLARIKKIMKADEDVRMISAEAPVIFAKACEMFILELTLRSWIHTEENKRRTLQKNDIAAAITRTDIFDFLVDIVPRDELKEEGIGIARAALPTMGAPADSGPYYYVPAQHQLAGPGMIMGKPVDQATTAAMYTAQPPHPVAYMWQQPQQQQAQQQQQMPDSG

SEQ ID NO: 150 - protein, 352 aa, Elaeis guineensis
MPLDNANAFDTQHFSNKDSEHSSVTSVHSASNCVDNFPSLWKQSGSHFPQSTYFKNFCMNMGFLAQPDNQMKQLGGQMPDQDSSSSQSTGQSHQEVSGTSEGNLHEQSISAQAGNDKTCGKQVEGHVNSVLFLGTPEAAFVSPRLDYGQSFACVPYTYADPSFGGVLAAYGSPAIIHPQMVGVPPSSRVPLPLEPAAEEPIYVNAKQYRAILRRRQLRAKLEAQNKLIKARKPYLHESRHLHAMKRARGSGGRFLNTKQLEQQQQRPLLPPPPSVSTGLGNLSASNLHFENGPSGSSAAPTSSADVVRVSTSGGMLEQQGHLSFLSADFHSHVRSTQGGGDSGSQPRITIMR

SEQ ID NO: 151 - protein, 300 aa, Glycine max
MQQIHSMPGGRFFSGSGSADRRLRPHHQNQQALKCPRCDSLNTKFCYYNNYNLSQPRHFCKNCRRYWTKGGVLRNVPVGGGCRKSKRSSKPNKITPSETASPPPPPHPDHNNNSNSHSSSESSSLTAAVATTTEAVSAPETLNSDSNNNNNMQESKLLIPALETNNPLEQGTGDCGGIFSEIGPFTSLITTTTSTNEPLGSGFGFGNSTLPDASSFQWHYQKVSSNNEELKLPENSFLDHTVDLSGMHSKTSHGGGFGSLDWQGGADQGLFDLPNTVDHAYWSHTHWSDHDNSSSLFHLP

SEQ ID NO: 152 - protein, 351 aa, Glycine max
MSSVFSEHKFQLQPSHQLLSLKKSLGDIDIPVPPRKLLTRRSAAVHDGSGDIYLPHSGSTDSSTDDDSDGDPYASDQFRMFEFKVRRCSRSRSHDWTDCPFVHPGEKARRRDPRRFYYSGTVCPEFRRGQCDRGDACEFSHGVFECWLHPSRYRTEACKDGKNCKRKVCFFAHTPRQLRVFHSNDNSNKKKCTDISPHNNNNCCLVCHCSNSTRSPTSTLFGMSHFSPPLSPPSPSSPSMFETNNHHHGVVKYNKDVFSELVCSMEGLNFDEASSLLSAASKPHHHNNLSSWLDVSKDHNQKQFNTLNSPTITACGSFSNNGNGGFLRAENGVVVDDVIAPDLAWVNELLM